(19) United States
(12) Patent Application Publication
(10) Pub. No.: US 2024/0117017 A1
(43) Pub. Date: Apr. 11, 2024

(54) HIV VACCINES AND METHODS OF MAKING AND USING

(71) Applicant: Gilead Sciences, Inc., Foster City, CA (US)

(72) Inventors: Xinan Liu, Shanghai (CN); Azure T. Makadzange, Half Moon Bay, CA (US); Stephen R. Martin, Tiburon, CA (US); Hesham Shehata, Sunnyvale, CA (US); Evguenia Svarovskaia, Redwood City, CA (US)

(21) Appl. No.: 18/468,515

(22) Filed: Sep. 15, 2023

Related U.S. Application Data
(62) Division of application No. 16/928,571, filed on Jul. 14, 2020, now Pat. No. 11,795,210.
(60) Provisional application No. 62/874,712, filed on Jul. 16, 2019.

(51) Int. Cl. C07K 16/10; A61K 9/14; A61K 45/06; C07K 16/28; C12N 15/10

(52) U.S. Cl. CPC C07K 16/1045; A61K 9/14; A61K 45/06; C07K 16/2818; C07K 16/2827; C12N 15/1082

(57) ABSTRACT

Provided are HIV-1 fusion polypeptides, polynucleotides encoding such fusion polypeptides, vectors expressing such fusion polypeptides for use in eliciting an immune response against HIV-1; pharmaceutical and immunogenic compositions and kits comprising such fusion polypeptides, polynucleotides or vectors, and methods of use in treating and/or preventing HIV-1. Further provided are methods for design of antiviral vaccines, including vaccines to elicit an immune response against HIV-1.

### CROSS-REFERENCE TO RELATED APPLICATIONS

This application claims the benefit under 35 U.S.C. § 119(e) of U.S. Provisional Patent Application No. 62/874,712, filed on Jul. 16, 2019, which is hereby incorporated herein by reference in its entirety for all purposes.

### SEQUENCE LISTING

The instant application contains a Sequence Listing which has been submitted electronically in ASCII format and is hereby incorporated by reference in its entirety. Said ASCII copy, created on Jun. 30, 2020, is named 1314_PC_SL.txt and is 446,538 bytes in size.

### BACKGROUND

Human immunodeficiency virus type 1 (HIV-1) infection is a serious, life-threatening disease and remains one of the leading causes of morbidity and mortality worldwide, with approximately 36.9 million people infected globally and 1.1 million people infected in the United States (US) (National Center for HIV/AIDS Viral Hepatitis STD & TB Prevention: Division of HIV/AIDS Prevention, HIV in the United States and Dependent Areas. January. 2019; UNAIDS, 2017 Global HIV Statistics. Fact Sheet—July. 2018). Combination antiretroviral therapy (cART) for HIV-1 infection has led to significant improvements in morbidity and mortality by suppressing viral replication, preserving immunologic function, and averting the progression to AIDS. However, despite cART, HIV-1 infection results in chronic immune activation and increased risk of non-AIDS-related morbidity and mortality.

In the early phase of infection, HIV-1 integrates into the genome of memory cluster determinant 4 (CD4) T cells, a subset of which forms a long-lived reservoir of HIV-1 infected cells that persist despite treatment with antiretroviral therapy (ART) (Siliciano, et al., Nature Medicine (2003) 9(6):727-728). Eradication of the viral reservoir is a component of any HIV cure strategy. Immune based therapies can be a further component of a combination approach to HIV cure or ART-free viral remission and can include T cell and antibody-based vaccines, passive administration of antibodies and immune modulators.

The development of HIV T cell specific vaccines has primarily focused in designing immunogens that provide universal coverage by addressing global HIV viral diversity. HIV-1 is defined by 4 groups (group M, N, O and P). Subtypes or clades (labeled A-K) and several cross clade recombinant forms within Group-M cause the majority of human disease. Strategies to design vaccines that address enormous global viral sequence diversity include in-silico designed polyvalent mosaic immunogens that capture common epitope variants within potential T cell epitopes (Fischer, et al., Nat Med, (2007) 13(1):100-6). These may be expressed as full-length artificial proteins or artificial recombinant proteins from regions with a high degree of sequence conservation (Ondondo, et al., Mol Ther, (2016) 24(4):832-42; Barouch, et al., Cell, (2013) 155(3):531-9). Subsequent iterations to the in-silico design algorithm led to the development of a computationally faster graph-based approach known as epigraph (Theiler, et al., Sci Rep, (2016) 6:33987). These design approaches can be used to develop a single global vaccine or be tailored to the clades circulating within a certain population and geography. These approaches focus exclusively on viral diversity, however, and do not consider the host genetic diversity that drives antigen presentation and T cell recognition, and subsequent emergence of immune-driven escape variants.

Antigen specific CD4+ and CD8+ T cells are associated with the control of viremia during acute infection and are associated with slow disease progression and control of viremia in individuals who maintain low viral load in the absence of ART (elite controllers). Antigen specific T cells recognize viral epitopes presented on MHC class I and II molecules. Human leukocyte antigen (HLA) class I alleles have been associated with HIV control in genome wide association studies (GWAS) (Fellay, et al., Science, 2007. 317(5840):944-7; International, H.I.V.C.S., et al., Science, (2010) 330(6010):1551-7). These proteins present antigenic peptides from sequences to induce effector and memory T cells. Current approaches to generating candidate vaccines for HIV-1 have focused on viral sequence diversity without adequately modelling the process of epitope generation across a range of host HLA alleles. This complex process of antigen presentation and T cell priming includes proteosomal cleavage, TAP transport, cross-presentation, MHC binding and peptide-MHC complex stability and ultimately TCR recognition (Yewdell, et al., Nat Rev Immunol, (2003) 3(12):952-61). Consequently, existing methods of generating T cell vaccines have had limited success, e.g., in some cases inducing on average only 4 responses per patient (see, e.g., Priddy, et al., Clin Infect Dis (2008) 46(11):1769-81; Sekaly, et al., J Exp Med. (2008) 205(1): 7-12; and Iaccino, et al., Retrovirology. (2008) 5:56).

In addition, highly variant viruses such as HIV-1 provide unique challenges due to the high level of sequence diversity and a host immune response that drives some of that sequence diversity. The role of the adaptive immune responses in driving diversity in HIV-1 has been well described, and results in changes in virus sequences over time (Goulder, et al., Nature, (2001) 412(6844):334-8; Kelleher, et al., J Exp Med, (2001) 193(3):375-86; Schneidewind, et al., J Virol, (2007) 81(22):12382-93; Kawashima, et al., Nature, (2009) 458(7238):641-5; Leslie, et al., Nat Med, (2004) 10(3):282-9; Phillips, et al., Nature, (1991) 354(6353):453-9). A large proportion of that diversity is driven by cytotoxic T lymphocytes that recognized peptide epitopes presented on MHC class I alleles. The selection pressure exerted by these T cell responses during chronic infection leads to HIV sequence adaptation. This sequence evolution drives the diversity of HIV-1 within individuals and across a population (Kawashima, et al., supra; Phillips, et al., supra). In addition, viral sequences are undergoing mutations that enable them to be hidden from host defenses. These sequences may resemble self-peptides or peptide sequences that induce central or peripheral tolerance. Standard vaccine design approaches may fail to account for viral sequence variants and may lead to inefficient use of vaccine capacity by including sequences that may induce responses that may cross react with self-antigens.

The impact of vaccines on human health cannot be overstated. Most of these are preventative vaccines, however, and have been effective in inducing usually neutralizing antibodies against infectious disease targets. The development of therapeutic vaccines has largely been advanced in cancer immunotherapeutics where the focus has been on developing vaccines that generate antigen specific T cells. Many tumor-associated or tumor-specific antigens are self-antigens and require the design of vaccines that need to overcome immune tolerance. Recent innovations in the identification and prediction of neoantigens that arise from cancer specific mutations, provide potential targets that may not be subject to central or peripheral tolerance mechanisms. Various informatics strategies have been established to support the identification of neoantigens and predict their ability to elicit strong T cell responses (see, e.g., Bulik-Sullivan, et al., Nature Biotech (2019) 37:55-63). In the development of therapeutic vaccines against HIV, the antigenic targets are defined by the virus. Tools to predict the capacity of those viral sequences to be effectively presented and stimulate an immune response are less well-defined. This is relevant within the context of HIV, where a high mutation rate coupled with host immune mediated selection pressure result in the establishment of highly variable quasi-species. We have therefore developed informatics tools that allow for the identification of conserved viral sequences in population-based consensus sequences or by individual deep sequencing of isolates and can predict presentation, priming of T cells and HLA driven escape pathways that are useful in designing HIV vaccine immunogens.

### SUMMARY

Provided herein are at least the following embodiments. Additional embodiments are described in the detailed embodiments and examples herein.

### Fusion Polypeptides

Embodiment 1: A fusion polypeptide comprising a plurality of polypeptide segments of one or more human immunodeficiency virus-1 (HIV-1) proteins encoded by one or more HIV genes selected from Gag, Nef, Env, Pol, Rev, Tat, Rev, Vif, Vpr and Vpu.

Embodiment 2: The fusion polypeptide of embodiment 1, wherein the plurality of polypeptide segments comprises or consists of only polypeptide segments encoded by HIV-1 genes Env, Gag, Nef and Pol, e.g. does not comprise polypeptide segments encoded by HIV-1 Tat, Rev, Vif, Vpr and/or Vpu genes.

Embodiment 3: The fusion polypeptide of embodiment 1, wherein the plurality of polypeptide segments comprises or consists of only polypeptide segments encoded by HIV-1 genes Gag, Nef and Pol, e.g. does not comprise polypeptide segments encoded by HIV-1 Env, Tat, Rev, Vif, Vpr and/or Vpu genes.

Embodiment 4: The fusion polypeptide of embodiment 1, wherein the plurality of polypeptide segments comprises or consists of only polypeptide segments encoded by HIV-1 genes Gag and Nef, e.g. does not comprise polypeptide segments encoded by HIV-1 Env, Pol, Tat, Rev, Vif, Vpr and/or Vpu genes.

Embodiment 5: The fusion polypeptide of embodiment 1, wherein the plurality of polypeptide segments comprises or consists of only polypeptide segments encoded by HIV-1 genes Pol and Nef, e.g. does not comprise polypeptide segments encoded by HIV-1 Env, Gag, Tat, Rev, Vif, Vpr and/or Vpu genes.

Embodiment 6: The fusion polypeptide of embodiment 1, wherein the plurality of polypeptide segments comprises or consists of only polypeptide segments encoded by HIV-1 genes Pol and Env, e.g. does not comprise polypeptide segments encoded by HIV-1 Gag, Nef, Tat, Rev, Vif, Vpr and/or Vpu genes.

Embodiment 7: The fusion polypeptide of embodiment 1, wherein the plurality of polypeptide segments comprises or consists of only polypeptide segments encoded by HIV-1 Pol gene, e.g. does not comprise polypeptide segments encoded by HIV-1 Env, Gag, Nef, Tat, Rev, Vif, Vpr and/or Vpu genes.

Embodiment 8: The fusion polypeptide of any one of embodiments 1 to 7, wherein the plurality of polypeptide segments does not contain a segment encoded by one, two, three or four of HIV Tat, Rev, Vif, Vpr and Vpu genes.

Embodiment 9: The fusion polypeptide of any one of embodiments 1 to 8, wherein the polypeptide segments are derived from conserved regions in a population of viral proteome sequences.

Embodiment 10: The fusion polypeptide of embodiment 9, wherein the conserved regions are greater than 80%, 81%, 82%, 83%, 84%, 85%, 86%, 87%, 88%, 89%, 90%, 91%, 92%, 93%, 94%, 95%, 96%, 97%, 98% or 99% conserved amongst HIV-1 species in interpatient populations.

Embodiment 11: The fusion polypeptide of any one of embodiments 9 to 10, wherein the conserved regions are conserved amongst one or more of HIV-1 clades A-K, e.g., one or more of clades A, B, C, D and G, or recombinant forms of one or more of HIV-1 clades A-K, and combinations thereof.

Embodiment 12: The fusion polypeptide of any one of embodiments 1 to 11, comprising at least 5 and up to 40 polypeptide segments, e.g. from 5 polypeptide segments and up to 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40 polypeptide segments.

Embodiment 13: The fusion polypeptide of any one of embodiments 1 to 12, wherein each polypeptide segment is at least 8 amino acids in length, and up to about 30, e.g., up to about 50, e.g., up to about 100, e.g., up to about 250 amino acids in length, e.g. from at least 8 amino acids in length up to 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 45, 50, 55, 60, 65, 70, 75, 80, 85, 90, 95, 100, 110, 120, 130, 140, 150, 160, 170, 180, 190, 200, 210, 220, 230, 240 or 250 amino acids in length.

Embodiment 14: The fusion polypeptide of any one of embodiments 1 to 13, wherein the full-length of the fusion polypeptide comprises at least about 350 amino acids and up to about 1000 amino acids, e.g., at least about 350 amino acids and up to about 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 or 1000 amino acids.

Embodiment 15: The fusion polypeptide of any one of embodiments 1 to 13, wherein the full-length of the fusion polypeptide comprises at least about 500 amino acids and up to about 1000 amino acids, e.g., at least about 500 amino acids and up to about 550, 560, 570, 580, 590, 600, 610, 620, 630, 640, 650, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 or 1000 amino acids.

Embodiment 16: The fusion polypeptide of any one of embodiments 1 to 13, wherein the full-length of the fusion polypeptide comprises or consists of only polypeptide segments encoded by HIV-1 genes Gag, Nef and Pol and is at least about 700 amino acids and up to about 800 amino acids, e.g., at least about 700 amino acids and up to about 710, 720, 730, 740, 750, 760, 770, 780, 790 or 800 amino acids in length (e.g., SEQ ID NOs: 345-350, 422-423 are illustrative fusion polypeptides).

Embodiment 17: The fusion polypeptide of any one of embodiments 1 to 13, wherein the full-length of the fusion polypeptide comprises or consists of only polypeptide segments encoded by HIV-1 genes Gag and Nef and is at least about 340 amino acids and up to about 500 amino acids, e.g., at least about 340 amino acids and up to about 350, 360, 370, 380, 390, 400, 410, 420, 430, 440, 450, 460, 470, 480, 490 or 500 amino acids in length, inclusive of an optional N-terminal signal peptide (e.g., SEQ ID NOs: 351-356, 430 are illustrative fusion polypeptides).

Embodiment 18: The fusion polypeptide of any one of embodiments 1 to 13, wherein the full-length of the fusion polypeptide comprises or consists of only polypeptide segments encoded by HIV-1 genes Pol and Env and is at least about 335 amino acids and up to about 970 amino acids, e.g., at least about 335 amino acids and up to about 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960 or 970, amino acids in length, inclusive of an optional N-terminal signal peptide (e.g., SEQ ID NOs: 357-366 are illustrative fusion polypeptides).

Embodiment 19: The fusion polypeptide of any one of embodiments 1 to 13, wherein the full-length of the fusion polypeptide comprises or consists of only polypeptide segments encoded by HIV-1 genes Pol and is at least about 645 amino acids and up to about 675 amino acids, e.g., at least about 645 amino acids and up to about 650, 655, 660, 670, 675 or 680 amino acids in length (e.g., SEQ ID NOs: 407-410 are illustrative fusion polypeptides).

Embodiment 20: The fusion polypeptide of any one of embodiments 1 to 13, wherein the full-length of the fusion polypeptide comprises or consists of only polypeptide segments encoded by HIV-1 genes Env, Gag, Nef and Pol, and is at least about 360 amino acids and up to about 510 amino acids, e.g., at least about 360 amino acids and up to about 370, 380, 390, 400, 410, 420, 430, 440, 450, 460, 470, 480, 490, 500 or 510 amino acids in length, inclusive of an optional N-terminal signal peptide (e.g., SEQ ID NOs: 367-371, 424, 431-435 are illustrative fusion polypeptides).

Embodiment 21: The fusion polypeptide of any one of embodiments 1 to 13, wherein the full-length of the fusion polypeptide comprises or consists of only polypeptide segments encoded by HIV-1 genes Env, Gag, Nef and Pol, and is at least about 760 amino acids and up to about 955 amino acids, e.g., at least about 760 amino acids and up to about 770, 780, 790, 800, 810, 820, 830, 840, 850, 860, 870, 880, 890, 900, 910, 920, 930, 940, 950, 955 amino acids in length, inclusive of an optional N-terminal signal peptide (e.g., SEQ ID NOs: 373-377, 411 are illustrative fusion polypeptides).

Embodiment 22: The fusion polypeptide of any one of embodiments 1 to 14, wherein the full-length of the fusion polypeptide is no longer than 800 amino acids, e.g. no longer than 795, 790, 785, 780, 775, 770, 765, 760, 755, 750, 745, 740, 735, 730, 725, 720, 715, 710, 705 or 700 amino acids.

Embodiment 23: The fusion polypeptide of any one of embodiments 1 to 22, wherein each polypeptide segment comprises or consists of one or more predicted T cell epitopes.

Embodiment 24: The fusion polypeptide of any one of embodiments 1 to 23, comprising one or more polypeptide segments that bind to or are presented by one or more human HLA class I alleles (e.g. 1, 2, 3, 4, 5 or 6 alleles), e.g. within a single subject or amongst multiple patients.

Embodiment 25: The fusion polypeptide of any one of embodiments 1 to 24, comprising one or more polypeptide segments that bind to or are presented by at least one human HLA class I molecule, e.g., by a human A*0201 HLA class I molecule.

Embodiment 26: The fusion polypeptide of any one of embodiments 1 to 25, comprising one or more 8-mer, 9-mer and/or 10-mer polypeptide segments that are presented by one or more human HLA class I alleles (e.g. 1, 2, 3, 4, 5 or 6 alleles), e.g. within a single subject.

Embodiment 27: The fusion polypeptide of any one of embodiments 1 to 25, comprising one or more 25-mer polypeptide segments, each 25-mer polypeptide segment comprising one or more 8-mer, 9-mer and/or 10-mer polypeptide segments that are presented by one or more human HLA class I alleles (e.g. 1, 2, 3, 4, 5 or 6 alleles), e.g. within a single subject.

Embodiment 28: The fusion polypeptide of any one of embodiments 1 to 27, comprising one or more polypeptide segments that are intracellularly processed and presented by one or more human HLA class II alleles (e.g. 1, 2, 3, 4, 5 or 6 alleles), e.g. within a single subject.

Embodiment 29: The fusion polypeptide of any one of embodiments 1 to 28, wherein one or more of the polypeptide segments is abutted or fused to an adjacent segment.

Embodiment 30: The fusion polypeptide of any one of embodiments 1 to 28, wherein one or more of the polypeptide segments is joined to an adjacent segment by one or more peptide linkers.

Embodiment 31: The fusion polypeptide of embodiment 30, wherein the one or more peptide linkers is selected from one or more of a polyalanine linker, a polyglycine linker, a cleavable linker, a flexible linker, a rigid linker, a Nef linking sequence, and combinations thereof.

Embodiment 32: The fusion polypeptide of embodiment 31, wherein the polyalanine linker comprises or consists of 2 or 3 contiguous alanine residues, e.g. AA, AAA (SEQ ID NO: 378), AAY (SEQ ID NO: 379) or AAX (SEQ ID NO: 380), wherein X is any amino acid (e.g. A, C, D, E, F, G, H, I, K, L, M, N, P, Q, R, S, T, V, Y).

Embodiment 33: The fusion polypeptide of embodiment 31, wherein the flexible linker or polyglycine linker comprises or consists of GG, GGS (SEQ ID NO: 419), GSG (SEQ ID NO: 420) or GGGS (SEQ ID NO: 421).

Embodiment 34: The fusion polypeptide of embodiment 31, wherein the cleavable linker is selected from a 2A cleavable peptide (e.g. foot-and-mouth disease virus (F2A), equine rhinitis A virus (E2A), porcine teschovirus-1 (P2A) and Thosea asigna virus (T2A)), a furin recognition/cleavage sequence (e.g. REKR (SEQ ID NO: 382), RRKR (SEQ ID NO: 383), RAKR (SEQ ID NO: 381)), a Nef linking sequence, and combinations, derivatives or variants thereof.

Embodiment 35: The fusion polypeptide of embodiment 34, wherein the cleavable linker comprises or consists of a furin recognition/cleavage site selected from the group consisting of RAKR (SEQ ID NO: 381), REKR (SEQ ID NO: 382) and RRKR (SEQ ID NO: 383).

Embodiment 36: The fusion polypeptide of any one of embodiments 34 to 35, wherein the cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388), or comprises or consists of the amino acid sequence of ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388).

Embodiment 37: The fusion polypeptide of embodiment 31, wherein the Nef linking sequence comprises or consists of an amino acid sequence that is at least 95%, 96%, 97%, 98% or 99% identical to VHAGPIA (SEQ ID NO: 389), VHAGPVA (SEQ ID NO: 390), or GALDI (SEQ ID NO:391), or comprises or consists of an amino acid sequence selected from VHAGPIA (SEQ ID NO: 389), VHAGPVA (SEQ ID NO: 390) and GALDI (SEQ ID NO: 391).

Embodiment 38: The fusion polypeptide of any one of embodiments 1 to 37, wherein the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 1-344.

Embodiment 39: The fusion polypeptide of any one of embodiments 1 to 38, wherein the plurality of polypeptide segments comprises one or more segments of one or more viral proteins, or fragments or subsequences thereof, encoded by the HIV-1 Gag gene.

Embodiment 40: The fusion polypeptide of embodiment 39, wherein the one or more viral proteins encoded by the HIV-1 Gag gene is selected from p7, p17 and p24, and wherein the fusion polypeptide does not comprise any p6 proteins.

Embodiment 41: The fusion polypeptide of any one of embodiments 39 to 40, wherein the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from:
SEQ ID NOs: 68-146 and 339-342;
SEQ ID NOs: 68, 69, 72, 73, 74, 75, 76, 77, 80, 81, 82, 83, 84, 85, 86, 87, 88, 89, 92, 93, 101, 102, 103, 104, 109, 110, 115, 116, 125, 126, 127, 128, 129, 130, 131, 132, 133, 134, 135, 136, 139, 140, 141, 142, 143, 144, 145 and 146;
SEQ ID NOs: 76, 77, 86, 87 and 92-124;
SEQ ID NOs: 76, 77, 86, 87, 94 and 95;
SEQ ID NOs: 76, 86 and 94;
SEQ ID NOs: 77, 87 and 95;
SEQ ID NOs: 68-79 and 92-124;
SEQ ID NOs: 70-71, 76-77 and 94-95;
SEQ ID NOs: 78, 79, 96, 99, 100, 107, 108, 113, 114, 121, 122, 123, 124, 137 and 138;
SEQ ID NOs: 78, 99, 107, 113, 121, 123 and 137;
SEQ ID NOs: 78, 79, 90, 91, 97, 98, 99, 100, 105, 106, 107, 108, 111, 112, 113, 114, 117, 118, 119, 120, 121, 122, 123, 124, 137 and 138;
SEQ ID NOs: 78, 90, 97, 105, 111, 117, 119 and 137; and
SEQ ID NOs: 78 and 137.

Embodiment 42: The fusion polypeptide of any one of embodiments 39 to 41, wherein the plurality of polypeptide segments comprises at least 2 polypeptide segments, e.g., at least 3, 4, 5, 6, 7, 8, 9, 10, 11, 12, 13, 14, 15, 16, 17, 18, 19, 20, or more, segments comprising or consisting of an HIV-1 Gag amino acid sequence corresponding to amino acid residue positions selected from 31-53, 37-51, 142-166, 175-199, 183-191, 257-282, 257-290, 265-282, 288-313, 288-321, 296-313, 333-357, 337-361, 341-349, 345-353 and 429-444, wherein the amino acid positions are with respect to SEQ ID NO:404.

Embodiment 43: The fusion polypeptide of any one of embodiments 39 to 42, wherein the plurality of polypeptide segments does not comprise 1, 2, 3, 4, 5, or more, polypeptide segments comprising or consisting of an HIV-1 Gag amino acid sequence corresponding to amino acid residue positions selected from 1-30, 54-127, 138-146, 370-428 and 445-500, or subsequences thereof, wherein the amino acid positions are with respect to SEQ ID NO:404.

Embodiment 44: The fusion polypeptide of any one of embodiments 39 to 43, wherein the plurality of polypeptide segments does not comprise 1, 2, 3, 4, 5, or more, polypeptide segments comprising or consisting of an HIV-1 Gag amino acid sequence of any one of SEQ ID NOs: 444-448, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 444-448, or subsequences thereof.

Embodiment 45: The fusion polypeptide of any one of embodiments 1 to 44, wherein the plurality of polypeptide segments comprises one or more segments of the viral protein encoded by the HIV-1 Nef gene.

Embodiment 46: The fusion polypeptide of embodiment 45, wherein the plurality of polypeptide segments comprises at least one polypeptide segment, e.g., at least 2, 3, 4, 5, 6, 7, 8, 9, 10, 11, 12, 13, 14, 15, 16, 17, 18, 19, 20, 21, 22, 23, 24, 25, or more, segments comprising or consisting of an amino acid sequence selected from:
SEQ ID NOs: 147, 148, 149, 150, 151, 152, 153, 154, 155, 156, 157, 158, 159, 160, 161, 162, 163, 164, 165, 166, 167, 168, 169, 170, 171 and 172;
SEQ ID NOs: 147, 148, 149, 150, 155, 156, 157, 158, 159, 160, 166, 167, 168, 169, 170 and 171;
SEQ ID NOs: 149-152;
SEQ ID NOs: 151 and 152;
SEQ ID NOs: 149, 150, 151, 152, 159, 160, 161, 162, 163, 164, 166, 167, 168, 169, 170, 171, 172, 173 and 174;
SEQ ID NOs: 151, 152, 161 and 162;
SEQ ID NOs: 151 and 152;
SEQ ID NOs: 153, 154, 172 and 173;
SEQ ID NOs: 153 and 172;
SEQ ID NOs: 153, 154, 155, 156, 157, 158, 163, 164, 165, 166, 167, 168, 169, 170, 171, 172 and 173;
SEQ ID NOs: 153 and 165; and
SEQ ID NO: 153.

Embodiment 47: The fusion polypeptide of any one of embodiments 45 to 46, wherein the plurality of polypeptide segments comprises at least 2 polypeptide segments, e.g., at least 3, 4, 5, 6, 7, 8, 9, 10, 11, 12, 13, 14, 15, or more, segments comprising or consisting of an HIV-1 Nef amino acid sequence corresponding to amino acid residue positions selected from 64-102, 81-102, 88-97, 91-99, 130-148, 130-154, 134-142, 134-148, 136-148, 137-145, 137-145 and 117-154, wherein the amino acid positions are with respect to SEQ ID NO:405.

Embodiment 48: The fusion polypeptide of any one of embodiments 45 to 47, wherein the plurality of polypeptide segments does not comprise 1, 2, 3, or more, polypeptide segments comprising or consisting of an HIV-1 Nef amino acid sequence corresponding to amino acid residue positions selected from 1-63, 103-116 and 155-206, or subsequences thereof, wherein the amino acid positions are with respect to SEQ ID NO:405.

Embodiment 49: The fusion polypeptide of any one of embodiments 45 to 48, wherein the plurality of polypeptide segments does not comprise 1, 2, 3, or more, polypeptide segments comprising or consisting of an HIV-1 Nef amino acid sequence of any one of SEQ ID NOs: 449-451, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 449-451, or subsequences thereof.

Embodiment 50: The fusion polypeptide of any one of embodiments 1 to 49, wherein the plurality of polypeptide segments comprises or consists of one or more segments of viral proteins encoded by the HIV-1 Gag and Nef genes.

Embodiment 51: The fusion polypeptide of embodiment 50, wherein the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from:
SEQ ID NOs: 68-79 and 92-124, 149, 150, 151, 152, 159, 160, 161, 162, 163, 164, 166, 167, 168, 169, 170, 171, 172, 173 and 174;
SEQ ID NOs: 70, 71, 76, 77, 94, 95, 151, 152, 161 and 162;
SEQ ID NOs: 70, 76, 94, 151 and 161; and
SEQ ID NOs: 71, 77, 95, 152 and 162.

Embodiment 52: The fusion polypeptide of any one of embodiments 1 to 4 and 8 to 51 comprising or consisting of the following polypeptide segments in sequential order, from N-terminus to C-terminus, optionally joined or connected by one or more linkers:
SEQ ID NOs: 70, 76, 94, 151 and 161; or
SEQ ID NOs: 71, 77, 95, 152 and 162.

Embodiment 53: The fusion polypeptide of any one of embodiments 1 to 52, wherein the plurality of polypeptide segments comprises or consists of an amino acid sequence of any one of SEQ ID NOs: 351-356 and 430, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 351-356 and 430.

Embodiment 54: The fusion polypeptide of any one of embodiments 1, 2, 6 and 9 to 53, wherein the plurality of polypeptide segments comprises one or more segments of one or more viral proteins encoded by the HIV-1 Env gene.

Embodiment 55: The fusion polypeptide embodiment 54, wherein the one or more viral proteins encoded by the HIV-1 Env gene is selected from gp120 and gp41.

Embodiment 56: The fusion polypeptide of any one of embodiments 54 to 55, wherein the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from:
SEQ ID NOs: 1-67 and 338;
SEQ ID NOs: 2, 3, 8, 9, 13, 14, 17, 18, 23, 24, 25, 26, 28, 29, 31, 32, 33, 34, 35, 36, 37, 38, 39, 40, 43, 44, 45, 46, 47, 48, 49, 50, 51, 52, 53, 54, 55, 56, 58, 59, 62, 63, 64, 65, 66 and 67;
SEQ ID NOs: 4, 5, 6, 7, 11, 12, 13, 14, 15, 16, 28, 29, 30, 37, 38, 41, 42, 43, 44, 45, 46, 47, 48, 49, 50, 51, 52, 53, 54, 55, 56, 57, 58, 59, 60, 61 and 338;
SEQ ID NOs: 4, 5, 6, 7, 11, 12, 13, 14, 15, 16, 28, 29, 30, 37, 38, 41 and 42;
SEQ ID NOs: 28, 29, 30 and 41-56;
SEQ ID NOs: 28, 29, 41 and 42;
SEQ ID NOs: 4, 5, 6, 7, 11, 12, 13, 14, 15, 16, 37 and 38;
SEQ ID NOs: 4, 5, 11, 12, 37 and 38;
SEQ ID NOs: 6, 7, 15, 16, 21, 22, 30, 60 and 61;
SEQ ID NOs: 6, 15, 21, 30 and 60;
SEQ ID NOs: 1, 2, 3, 4, 5, 6, 7, 10, 11, 12, 13, 14, 15, 16, 19, 20, 27, 55, 56, 57, 58, 59, 60, 61 and 338;
SEQ ID NOs: 1, 10, 19, 27, 55, 56 and 57; and
SEQ ID NOs: 6, 15 and 60.

Embodiment 57: The fusion polypeptide of any one of embodiments 54 to 56, wherein the plurality of polypeptide segments comprises at least 2 polypeptide segments, e.g., at least 3, 4, 5, 6, 7, 8, 9, 10, 11, 12, 13, 14, or more, segments comprising or consisting of an HIV-1 Env amino acid sequence corresponding to amino acid residue positions selected from 28-52, 34-48, 34-47, 36-44, 59-83, 64-83, 66-83, 67-75, 113-137, 235-259, 586-594, 586-610, 589-606 and 594-602, wherein the amino acid positions are with respect to SEQ ID NO:403.

Embodiment 58: The fusion polypeptide of any one of embodiments 54 to 57, wherein the plurality of polypeptide segments does not comprise 1, 2, 3, 4, 5, 6, or more, polypeptide segments comprising or consisting of an HIV-1 Env amino acid sequence corresponding to amino acid residue positions selected from 1-27, 53-58, 84-112, 138-234, 269-474, 490-501, 611-856, or subsequences thereof, wherein the amino acid positions are with respect to SEQ ID NO:403.

Embodiment 59: The fusion polypeptide of any one of embodiments 54 to 57, wherein the plurality of polypeptide segments does not comprise 1, 2, 3, 4, 5, or more, polypeptide segments comprising or consisting of an HIV-1 Env amino acid sequence of any one of SEQ ID NOs: 437-443, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 437-443, or subsequences thereof.

Embodiment 60: The fusion polypeptide of any one of embodiments 1 to 58, wherein the plurality of polypeptide segments comprises or consists of one or more segments of one or more viral proteins encoded by the HIV-1 Pol gene.

Embodiment 61: The fusion polypeptide of embodiment 60, wherein the one or more viral proteins encoded by the HIV-1 Pol gene is selected from one or more of protease (PR), reverse transcriptase (RT), and integrase (INT).

Embodiment 62: The fusion polypeptide of any one of embodiments 60 to 61, wherein the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from:
SEQ ID NOs: 174-337 and 343-344;
SEQ ID NOs: 174, 175, 178, 179, 180, 181, 182, 183, 184, 185, 193, 194, 195, 196, 197, 198, 199, 200, 203, 204, 205, 206, 207, 208, 213, 214, 221, 222, 236, 237, 245, 246, 247, 248, 249, 250, 251, 252, 253, 254, 255, 256, 257, 258, 263, 264, 266, 267, 268, 269, 270, 271, 272, 273, 278, 279, 280, 281, 282, 283, 284, 285, 286, 287, 288, 289, 290, 291, 292, 293, 294, 295, 305, 306, 307, 308, 309, 310, 313, 314, 315, 316, 317, 318, 321 and 322;
SEQ ID NOs: 180, 181, 182, 183, 184, 185, 186, 187, 190, 191, 192, 193, 194, 195, 196, 221, 222, 294, 295, 296, 297, 298, 299, 300, 301, 305, 306, 307, 308, 311, 312, 321, 322, 323, 324, 325, 326, 327, 328, 329, 330, 331, 332, 333, 334, 335, 336 and 337;
SEQ ID NOs: 180, 181, 186, 187, 221, 222, 294, 295, 307, 308, 321 and 322;
SEQ ID NOs: 176, 177, 178, 179, 180, 181, 182, 183, 184, 185, 186, 187, 188, 189, 190, 191, 192, 193, 194, 195, 196, 197, 198, 199, 200, 201, 202, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 257, 258, 259, 260, 261, 262, 263, 264, 265, 266, 267, 282, 283, 294, 295, 296, 297, 298, 299, 300, 301, 302, 305, 306, 319, 320, 321, 322, 323, 324, 325, 326, 327, 328, 329, 330, 331, 332, 333, 334, 335, 336 and 337;
SEQ ID NOs: 176, 177, 188, 189, 213, 214, 223, 224, 259, 260, 282, 283, 294, 295, 305, 306, 319 and 320;
SEQ ID NOs: 180, 181, 186, 187, 221, 222, 294, 295, 321 and 322;
SEQ ID NOs: 182-202, 292-302, 305 and 306;
SEQ ID NOs: 188, 189, 294, 295, 305 and 306;
SEQ ID NOs: 176, 177, 178, 179, 180, 181, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 257, 258, 259, 260, 261, 262, 263, 264, 265, 266, 267, 282, 283, 319, 320, 321, 322, 323, 324, 325, 326, 327, 328, 329, 330, 331, 332, 333, 334, 335, 336 and 337;
SEQ ID NOs: 176, 177, 213, 214, 223, 224, 259, 260, 282, 283, 319 and 320;
SEQ ID NOs: 192, 201, 202, 215, 216, 217, 218, 219, 220, 229, 230, 231, 240, 241, 242, 243, 244, 265, 276, 277, 298, 299, 302, 311, 312, 327, 328, 331, 332, 333, 336 and 337;
SEQ ID NOs: 192, 201, 215, 217, 219, 229, 230, 240, 241, 243, 265, 276, 298, 302, 311, 327, 331, 333 and 336;
SEQ ID NOs: 190, 191, 192, 197, 198, 209, 210, 211, 212, 213, 214, 215, 216, 217, 218, 219, 220, 225, 226, 227, 228, 229, 230, 231, 232, 233, 234, 235, 238, 239, 261, 262, 274, 275, 276, 277, 296, 297, 298, 299, 300, 301, 302, 303, 304, 323, 324, 325, 326, 327, 328, 329, 330, 331, 332, 333, 334, 335, 336, 337, 343, 344, 375 and 376;
SEQ ID NOs: 190, 197, 209, 210, 211, 225, 227, 234, 238, 261, 296, 300, 303, 323, 325, 329 and 334; and
SEQ ID NOs: 192, 215, 217, 219, 229, 230, 276, 298, 302, 327, 331, 333 and 336.

Embodiment 63: The fusion polypeptide of any one of embodiments 54 to 62, wherein the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from:
SEQ ID NOs: 4, 5, 6, 7, 11, 12, 13, 14, 15, 16, 28, 29, 30, 37, 38, 41, 42, 43, 44, 45, 46, 47, 48, 49, 50, 51, 52, 53, 54, 55, 56, 57, 58, 59, 60, 61, 176, 177, 178, 179, 180, 181, 182, 183, 184, 185, 186, 187, 188, 189, 190, 191, 192, 193, 194, 195, 196, 197, 198, 199, 200, 201, 202, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 257, 258, 259, 260, 261, 262, 263, 264, 265, 266, 267, 282, 283, 294, 295, 296, 297, 298, 299, 300, 301, 302, 305, 306, 319, 320, 321, 322, 323, 324, 325, 326, 327, 328, 329, 330, 331, 332, 333, 334, 335, 336, 337 and 338;
SEQ ID NOs: 4, 5, 6, 7, 11, 12, 13, 14, 15, 16, 28, 29, 30, 37, 38, 41, 42, 176, 177, 188, 189, 213, 214, 223, 224, 259, 260, 282, 283, 294, 295, 305, 306, 319 and 320;
SEQ ID NOs: 28, 29, 30, 41-56, 182-202, 292-302, 305 and 306;
SEQ ID NOs: 28, 29, 41, 42, 188, 189, 294, 295, 305 and 306;
SEQ ID NOs: 4, 5, 6, 7, 11, 12, 13, 14, 15, 16, 37, 38, 176, 177, 178, 179, 180, 181, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 257, 258, 259, 260, 261, 262, 263, 264, 265, 266, 267, 282, 283, 319, 320, 321, 322, 323, 324, 325, 326, 327, 328, 329, 330, 331, 332, 333, 334, 335, 336 and 337; and
SEQ ID NOs: 4, 5, 11, 12, 37, 38, 176, 177, 213, 214, 223, 224, 259, 260, 282, 283, 319 and 320.

Embodiment 64: The fusion polypeptide of any one of embodiments 1, 6, 9 to 38 and 54 to 63, comprising or consisting of the following polypeptide segments in sequential order, from N-terminus to C-terminus, optionally joined or connected by one or more linkers:
SEQ ID NOs: 188, 305, 28, 41, 294, 4, 176, 11, 319, 259, 282, 223, 213 and 37;
SEQ ID NOs: 188, 305, 28, 41 and 294;
SEQ ID NOs: 4, 176, 11, 319, 259, 282, 223, 213 and 37;
SEQ ID NOs: 189, 306, 29, 42, 295, 5, 177, 12, 320, 260, 283, 224, 214 and 38;
SEQ ID NOs: 189, 306, 29, 42 and 295;
SEQ ID NOs: 5, 177, 12, 320, 260, 283, 224, 214 and 38;
SEQ ID NOs: 305, 319, 259, 282, 223, 213, 294, 176 and 188;
SEQ ID NOs: 306, 320, 260, 283, 224, 214, 295, 177 and 189;
SEQ ID NOs: 305, 294, 223, 213, 176, 259, 319, 188 and 282;
SEQ ID NOs: 306, 295, 224, 214, 177, 260, 320, 189 and 283;
SEQ ID NOs: 305, 294, 319, 259, 282, 223, 176, and 188;
SEQ ID NOs: 306, 295, 320, 260, 283, 224, 177 and 189;
SEQ ID NOs: 305, 223, 294, 176, 259, 319, 188 and 282; or
SEQ ID NOs: 306, 224, 295, 177, 260, 320, 189 and 283.

Embodiment 65: The fusion polypeptide of any one of embodiments 60 to 64, wherein the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 39, 30, or more, segments comprising or consisting of an HIV-1 Pol amino acid sequence corresponding to amino acid residue positions selected from 144-168, 152-160, 291-315, 326-350, 328-352, 330-354, 333-354, 334-342, 336-344, 338-346, 374-398, 380-404, 382-390, 388-396, 399-423, 400-424, 406-430, 553-577, 642-666, 650-658, 759-783, 767-775, 768-792, 776-784, 834-858, 940-964, 947-971, 948-956, 948-972, 955-963, 956-964, 980-1003 and 988-996, wherein the amino acid positions are with respect to SEQ ID NO:406.

Embodiment 66: The fusion polypeptide of any one of embodiments 60 to 65, wherein the plurality of polypeptide segments does not comprise 1, 2, 3, 4, 5, 6, 7, 8, 9, 10, or more, polypeptide segments comprising or consisting of an HIV-1 Pol amino acid sequence corresponding to amino acid residue positions selected from 1-55, 118-128, 321-325, 355-366, 432-541, 607-641, 667-682, 709-746, 828-833, 921-930, or subsequences thereof, wherein the amino acid positions are with respect to SEQ ID NO:406.

Embodiment 67: The fusion polypeptide of any one of embodiments 60 to 66, wherein the plurality of polypeptide segments does not comprise 1, 2, 3, 4, 5, or more, polypeptide segments comprising or consisting of an HIV-1 Pol amino acid sequence of any one of SEQ ID NOs: 452-461, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 452-461, or subsequences thereof.

Embodiment 68: The fusion polypeptide of any one of embodiments 1, 6 to 38, and 54 to 67, wherein the plurality of polypeptide segments comprises or consists of an amino acid sequence of any one of SEQ ID NOs: 357-366 and 407-410, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 357-366 and 407-410.

Embodiment 69: The fusion polypeptide of any one of embodiments 1, 3 and 8 to 68, wherein the plurality of polypeptide segments comprises or consists of segments of viral proteins encoded by Gag, Nef and Pol genes.

Embodiment 70: The fusion polypeptide of embodiment 69, wherein the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from:
SEQ ID NOs: 76, 77, 86, 87, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 149, 150, 151, 152, 180, 181, 182, 183, 184, 185, 186, 187, 190, 191, 192, 193, 194, 195, 196, 221, 222, 294, 295, 296, 297, 298, 299, 300, 301, 305, 306, 307, 308, 311, 312, 321, 322, 323, 324, 325, 326, 327, 328, 329, 330, 331, 332, 333, 334, 335, 336, 337, 339, 340, 341 and 342; and
SEQ ID NOs: 76, 77, 86, 87, 94, 95, 151, 152, 181, 182, 186, 187, 221, 222, 294, 195, 307, 308, 321, 322.

Embodiment 71: The fusion polypeptide of any one of embodiments 69 to 70, comprising or consisting of the following polypeptide segments in sequential order, from N-terminus to C-terminus, optionally joined or connected by one or more linkers:
SEQ ID NOs: 76, 86, 94, 180, 186, 221, 294, 307, 321 and 151; or
SEQ ID NOs: 77, 87, 95, 181, 187, 222, 295, 308, 322 and 152.

Embodiment 72: The fusion polypeptide of any one of embodiments 69 to 71, wherein the plurality of polypeptide segments comprises or consists of an amino acid sequence of any one of SEQ ID NOs: 345-350, the sequences in Table 1, and SEQ ID NOs: 422-424, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NO: 345-350, the sequences in Table 1, and SEQ ID NOs: 422-424.

Embodiment 73: The fusion polypeptide of any one of embodiments 1 to 72, wherein the plurality of polypeptide segments comprises or consists of segments of viral proteins encoded by Gag, Pol, Env, and Nef genes, wherein each of the plurality of polypeptide segments can bind to or be presented by a human HLA allele A*0201.

Embodiment 74: The fusion polypeptide of embodiment 73, wherein each of the plurality of polypeptide segments are from 8-35 amino acids in length, e.g. from 9-34 amino acids in length, e.g. from 9-25 amino acids in length.

Embodiment 75: The fusion polypeptide of any one of embodiments 73 to 74, wherein the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from
SEQ ID NOs: 6, 7, 15, 16, 21, 22, 30, 60, 61, 78, 79, 96, 99, 100, 107, 108, 113, 114, 121, 122, 123, 124, 137, 138, 153, 154, 172, 173, 192, 201, 202, 215, 216, 217, 218, 219, 220, 229, 230, 231, 240, 241, 242, 243, 244, 265, 276, 277, 298, 299, 302, 311, 312, 327, 328, 331, 332, 333, 336, and 337;
SEQ ID NOs: 6, 15, 21, 30, 60, 78, 99, 107, 113, 121, 123, 137, 153, 172, 192, 201, 215, 217, 219, 229, 230, 240, 241, 243, 265, 276, 298, 302, 311, 327, 331, 333 and 336;
SEQ ID NOs: 1, 2, 3, 4, 5, 6, 7, 10, 11, 12, 13, 14, 15, 16, 19, 20, 27, 55, 56, 57, 58, 59, 60, 61, 78, 79, 90, 91, 97, 98, 99, 100, 105, 106, 107, 108, 111, 112, 113, 114, 117, 118, 119, 120, 121, 122, 123, 124, 137, 138, 153, 154, 155, 156, 157, 158, 163, 164, 165, 166, 167, 168, 169, 170, 171, 172, 173, 190, 191, 192, 197, 198, 209, 210, 211, 212, 213, 214, 215, 216, 217, 218, 219, 220, 225, 226, 227, 228, 229, 230, 231, 232, 233, 234, 235, 238, 239, 261, 262, 274, 275, 276, 277, 296, 297, 298, 299, 300, 301, 302, 303, 304, 323, 324, 325, 326, 327, 328, 329, 330, 331, 332, 333, 334, 335, 336, 337, 338, 343 and 344;
SEQ ID NOs: 1, 10, 19, 27, 55, 56, 57, 78, 90, 97, 105, 111, 117, 119, 137, 153, 165, 190, 197, 209, 210, 211, 225, 227, 234, 238, 261, 296, 300, 303, 323, 325, 329 and 334.

Embodiment 76: The fusion polypeptide of any one of embodiments 73 to 75, comprising or consisting of the following polypeptide segments in sequential order, from N-terminus to C-terminus, optionally joined or connected by one or more linkers:
SEQ ID NOs: 201, 78, 107, 96, 229, 172, 327, 6, 333, 243, 331, 192, 265, 311, 137, 15, 123, 30, 336, 302, 153, 219, 298, 121, 230, 240, 60, 241, 276, 113, 99, 21, 217 and 215;
SEQ ID NOs: 78, 296, 1, 339, 197, 329, 232, 323, 303, 234, 90, 261, 274, 238, 211, 325, 137, 227, 209, 190, 341, 57, 225, 27, 210, 119, 19, 165, 334, 117, 153, 10, 97 and 300; or
SEQ ID NOs: 296, 1, 78, 197, 339, 227, 261, 274, 238, 325, 137, 329, 303, 234, 90, 232, 27, 57, 225, 323, 190, 341, 119, 19, 165, 334, 117, 153, 10, 97 and 300.

Embodiment 77: The fusion polypeptide of any one of embodiments 73 to 76, wherein the plurality of polypeptide segments comprises or consists of an amino acid sequence of any one of SEQ ID NOs: 367-377, 411, 431-435, or a sequence that is 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 367-377, 411, 431-435.

Embodiment 78: The fusion polypeptide of any one of embodiments 1 to 77, wherein the fusion polypeptide does not comprise the amino acid sequence YMDD (SEQ ID NO: 462) or YVDD (SEQ ID NO: 463).

Embodiment 79: The fusion polypeptide of embodiment 78, wherein the fusion polypeptide does not comprise one or more amino acid sequences selected from SEQ ID NOs: 215, 216, 217, 218, 219 and 220.

Embodiment 80: The fusion polypeptide of any one of embodiments 78 to 79, wherein the fusion polypeptide does not comprise one or more amino acid sequences selected from SEQ ID NOs: 209, 210, 211, 212, 213, 214, 343 and 344.

Embodiment 81: A fusion polypeptide comprising an amino acid sequence of SEQ ID NOs: 345-352, 357-362, 367, 373, 407-411 or 422-424, or a sequence that is at least 85%, 86%, 87%, 88%, 89%, 90%, 91%, 92%, 93%, 94%, 95%, 96%, 97%, 98%, or 99% identical to any one of SEQ ID NOs: 345-352, 357-362, 367, 373, 407-411, 422-424 and 431-435.

Embodiment 82: The fusion polypeptide of any one of embodiments 1 to 81, comprising an N-terminal signal peptide or leader sequence.

Embodiment 83: The fusion polypeptide of embodiment 82, wherein the signal peptide or leader sequence is from a source protein selected from a serum protein, a cytokine, a chemokine, a chaperone protein, an invariant protein, and a protein that directs proteins to the lysosomal compartment.

Embodiment 84: The fusion polypeptide of any one of embodiments 82 to 83, wherein the signal peptide or leader sequence is from a source protein selected from the group consisting of: colony stimulating factor 2 (CSF2, GM-CSF), tissue type plasminogen activator (PLAT, t-PA), C-C motif chemokine ligand 7 (CCL7, MCP-3), C-X-C motif chemokine ligand 10 (CXCL10, IP-10), catenin beta 1 (CTNNB1), CD74 (p33; DHLAG; HLADG; Ia-GAMMA, invariant chain), serum albumin (ALB), polyubiquitin B/C (UBB/UBC), calreticulin (CALR), vesicular stomatitis virus G protein (VSV-G), lysosomal associated membrane protein 1 (LAMP-1) and lysosomal associated membrane protein 2 (LAMP-2).

Embodiment 85: The fusion polypeptide of any one of embodiments 82 to 84, wherein the signal peptide or leader sequence is selected from an amino acid sequence of any one of SEQ ID NOs: 393-402 and 412-413, or a sequence that is at least 95%, 96%, 97%, 98%, or 99% identical to any one of SEQ ID NOs: 393-402 and 412-413.

Embodiment 86: The fusion polypeptide of any one of embodiments 1 to 85, wherein the fusion polypeptide is recombinantly produced or chemically synthesized.

Embodiment 87: The fusion polypeptide of any one of embodiments 1 to 86, wherein the fusion polypeptide is capable of inducing, promoting or stimulating an immune response in a human.

Embodiment 88: The fusion polypeptide of any one of embodiments 1 to 87, wherein the fusion polypeptide is capable of inducing, promoting or stimulating an immune response against HIV-1 in a human.

Embodiment 89: The fusion polypeptide of any one of embodiments 1 to 88, wherein the fusion polypeptide is capable of inducing, promoting or stimulating proliferation and/or activation of one or more cell types selected from monocyte-derived dendritic cells (DCs), CD8+ T cells and CD4+ T cells.

Polynucleotides, Lipoplexes, Expression Cassettes. Vectors. Host Cells

Embodiment 90: A polynucleotide encoding one or more fusion polypeptides of any one of embodiments 1 to 89.

Embodiment 91: The polynucleotide of embodiment 90, wherein the polynucleotide comprises or is in the form of cDNA, mRNA, self-amplifying RNA (SAM), self-replicating RNA, or self-amplifying replicon RNA (RepRNA).

Embodiment 92: The polynucleotide of embodiment 91, wherein the polynucleotide comprises one or more self-replicating or self-amplifying alphavirus replicons.

Embodiment 93: The polynucleotide of any one of embodiments 90 to 92, comprising a nucleic acid sequence of any one of SEQ ID NOs: 414-418, or that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 414-418.

Embodiment 94: A lipoplex, e.g., a lipid nanoparticle (LNP), comprising the polynucleotide of any one of embodiments 90 to 93.

Embodiment 95: An expression cassette, comprising a polynucleotide of any one of embodiments 90 to 93 operably linked to one or more regulatory sequences.

Embodiment 96: The expression cassette of embodiment 95, wherein the polynucleotide is operably linked to and under the control of a constitutive promoter.

Embodiment 97: The expression cassette of any of embodiments 95 to 96, wherein the promoter is selected from a CMV promoter, a CAG promoter and an EF1a promoter.

Embodiment 98: A vector comprising one or more polynucleotides of any one of embodiments 90 to 93, or an expression cassette of any one of embodiments 95 to 97.

Embodiment 99: The vector of embodiment 98, wherein the vector is a plasmid vector, a bacterial vector or a viral vector.

Embodiment 100: The vector of any one of embodiments 98 to 99, wherein the vector is a viral vector or a viral expression vector.

Embodiment 101: The vector of any one of embodiments 98 to 100, wherein the viral vector or viral expression vector is from a DNA virus or an RNA virus.

Embodiment 102: The vector of any one of embodiments 98 to 101, wherein the viral vector or viral expression vector is from a virus selected from the group consisting of adenovirus, adeno-associated virus, arenavirus, alphavirus, poxvirus, cytomegalovirus, rhabdovirus, vesicular stomatitis virus, flavivirus, maraba virus and vaccinia virus.

Embodiment 103: The vector of any one of embodiments 98 to 102, wherein the viral vector or the viral expression vector is from a virus from a taxonomical family selected from Adenoviridae, Arenaviridae, Herpesviridae (e.g. Cytomegalovirus), Poxviridae (e.g. Vaccinia virus, e.g. modified vaccinia Ankara (MVA)), Paramyxoviridae (e.g. measles virus), Flaviviridae (e.g. Yellow fever virus), Rhabdoviridae (e.g. Vesiculovirus, e.g. Maraba vesiculovirus), Togaviridae (e.g., Alphavirus).

Embodiment 104: The vector of any one of embodiments 98 to 103, wherein the viral vector or viral expression vector is an arenavirus vector selected from Lymphocytic choriomeningitis mammarenavirus (LCMV), Cali mammarenavirus (a.k.a., Pichinde mammarenavirus or Pichinde arenavirus), Guanarito virus (GTOV), Junin virus (JUNV), Lassa virus (LASV), Lujo virus (LUJV), Machupo virus (MACV), Sabia virus (SABV), and Whitewater Arroyo virus (WWAV).

Embodiment 105: The vector of embodiment 104, wherein the viral vector or viral expression vector is an arenavirus vector selected from Lymphocytic choriomeningitis mammarenavirus (LCMV) or Cali mammarenavirus (a.k.a. Pichinde mammarenavirus or Pichinde arenavirus).

Embodiment 106: The vector of any one of embodiments 98 to 103, wherein the viral vector or viral expression vector is a human adenovirus or a simian adenovirus (e.g., a chimpanzee adenovirus, a gorilla adenovirus or a rhesus adenovirus).

Embodiment 107: The vector of embodiment 106, wherein the viral vector or viral expression vector is an adenovirus vector selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (e.g. ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), ChAd147 (AdC147)), gorilla adenovirus (e.g. GC44, GC45, GC46) and rhesus adenovirus (e.g., RhAd51, RhAd52, RhAd53, RhAd54, RhAd55, RhAd56, RhAd57, RhAd58, RhAd59, RhAd60, RhAd61, RhAd62, RhAd63, RhAd64, RhAd65, RhAd66).

Embodiment 108: The vector of any one of embodiments 98 to 107, wherein the viral vector or viral expression vector is replication defective, replication deficient, replication attenuated or replication competent.

Embodiment 109: The vector of any one of embodiments 98 to 108, wherein the viral vector or viral expression vector is an adenoviral vector comprising one or more polynucleotides that encode one or more fusion proteins comprising an amino acid sequence of any one of any one of SEQ ID NOs: 345-377, 407-411, 422-424, 430-435, or that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 345-377, 407-411, 422-424, 430-435.

Embodiment 110: The vector of any one of embodiments 98 to 109, wherein the vector comprises two or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the following amino acid sequences: SEQ ID NOs: 345 and 346; SEQ ID NOs: 347 and 348; SEQ ID NOs: 349 and 350; SEQ ID NOs: 351 and 352; SEQ ID NOs: 430 and 352; SEQ ID NOs: 357 and 358; SEQ ID NOs: 360 and 362; SEQ ID NOs: 359 and 361; SEQ ID NOs: 351 and 357; SEQ ID NOs: 351 and 358; SEQ ID NOs: 351 and 359; SEQ ID NOs: 351 and 360; SEQ ID NOs: 351 and 361; SEQ ID NOs: 351 and 362; SEQ ID NOs: 351 and 407; SEQ ID NOs: 351 and 408; SEQ ID NOs: 351 and 409; SEQ ID NOs: 351 and 410; SEQ ID NOs: 352 and 357; SEQ ID NOs: 352 and 358; SEQ ID NOs: 352 and 359; SEQ ID NOs: 352 and 360; SEQ ID NOs: 352 and 361; SEQ ID NOs: 352 and 362; SEQ ID NOs: 352 and 407; SEQ ID NOs: 352 and 408; SEQ ID NOs: 352 and 409; SEQ ID NOs: 352 and 410; SEQ ID NOs: 430 and 357; SEQ ID NOs: 430 and 358; SEQ ID NOs: 430 and 359; SEQ ID NOs: 430 and 360; SEQ ID NOs: 430 and 361; SEQ ID NOs: 430 and 362; SEQ ID NOs: 407 and 409; SEQ ID NOs: 407 and 408; SEQ ID NOs: 408 and 410; or SEQ ID NOs: 409 and 410.

Embodiment 111: A host cell comprising one or more polynucleotides of any one of embodiments 90 to 93, or one or more vectors of any one of embodiments 98 to 110.

Embodiment 112: The host cell of embodiment 111, wherein the one or more polynucleotides are not integrated into the host cell genome, e.g., are episomal.

Embodiment 113: The host cell of embodiment 111, wherein the one or more polynucleotides are integrated into the host cell genome.

Embodiment 114: The host cell of any one of embodiments 111 to 113, wherein the host cell is a mammalian cell, e.g., a human cell, e.g., a cell line selected from BHK-21, A549, Vero, HEK293 (e.g., HEK293E, HEK293F, HEK293H, HEK293T, Expi293™) cells, MDCK, Caco-2 and Calu-3.

Embodiment 115: The host cell of any one of embodiments 111 to 114, wherein the host cell is in vitro.

Embodiment 116: The host cell of any one of embodiments 111 to 114, wherein the host cell is in vivo.

### Compositions

Embodiment 117: An immunogenic composition comprising one or more of the fusion polypeptides of any one of embodiments 1 to 89, or one or more polynucleotides of any one of embodiments 90 to 93, or one or more vectors of any one of embodiments 98 to 110, and a pharmaceutically acceptable carrier.

Embodiment 118: The immunogenic composition of embodiment 117, comprising two or more of the fusion polypeptides of any one of embodiments 1 to 89, or two or more polynucleotides of any one of embodiments 90 to 93, or two or more vectors of any one of embodiments 98 to 110.

Embodiment 119: The immunogenic composition of any one of embodiments 117 to 118, wherein the one or more polynucleotides comprise or are in the form of DNA, cDNA, mRNA, or self-replicating RNA.

Embodiment 120: The immunogenic composition of any one of embodiments 117 to 118, comprising:
1) One or more fusion polypeptides comprising or consisting of the following polypeptide segments in sequential order, from N-terminus to C-terminus, optionally joined or connected by one or more linkers:
SEQ ID NOs: 70, 76, 94, 151 and 161; or
SEQ ID NOs: 71, 77, 95, 152 and 162; and
2) One or more fusion polypeptides comprising or consisting of the following polypeptide segments in sequential order, from N-terminus to C-terminus, optionally joined or connected by one or more linkers:
SEQ ID NOs: 188, 305, 28, 41, 294, 4, 176, 11, 319, 259, 282, 223, 213 and 37;
SEQ ID NOs: 188, 305, 28, 41 and 294;
SEQ ID NOs: 4, 176, 11, 319, 259, 282, 223, 213 and 37;
SEQ ID NOs: 189, 306, 29, 42, 295, 5, 177, 12, 320, 260, 283, 224, 214, and 38;
SEQ ID NOs: 189, 306, 29, 42 and 295;
SEQ ID NOs: 5, 177, 12, 320, 260, 283, 224, 214 and 38;
SEQ ID NOs: 305, 319, 259, 282, 223, 213, 294, 176 and 188;
SEQ ID NOs: 306, 320, 260, 283, 224, 214, 295, 177 and 189;
SEQ ID NOs: 305, 294, 223, 213, 176, 259, 319, 188 and 282;
SEQ ID NOs: 306, 295, 224, 214, 177, 260, 320, 189 and 283;
SEQ ID NOs: 305, 294, 319, 259, 282, 223, 176, and 188;
SEQ ID NOs: 306, 295, 320, 260, 283, 224, 177 and 189;
SEQ ID NOs: 305, 223, 294, 176, 259, 319, 188 and 282; or
SEQ ID NOs: 306, 224, 295, 177, 260, 320, 189 and 283.

Embodiment 121: The immunogenic composition of any one of embodiments 117 to 120, comprising one or more adenoviral vectors, each adenoviral vector comprising one or more polynucleotides encoding one or more fusion proteins comprising an amino acid sequence of any one of any one of SEQ ID NOs: 345-377, 407-411, 422-424, 430-435, or that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 345-377, 407-411, 422-424, 430-435.

Embodiment 122: The immunogenic composition of any one of embodiments 117 to 121, comprising one or more viral vectors, each viral vector comprising one or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the following amino acid sequences: SEQ ID NOs: 345 and 346; SEQ ID NOs: 347 and 348; SEQ ID NOs: 349 and 350; SEQ ID NOs: 351 and 352; SEQ ID NOs: 430 and 352; SEQ ID NOs: 357 and 358; SEQ ID NOs: 360 and 362; SEQ ID NOs: 359 and 361; SEQ ID NOs: 351 and 357; SEQ ID NOs: 351 and 358; SEQ ID NOs: 351 and 359; SEQ ID NOs: 351 and 360; SEQ ID NOs: 351 and 361; SEQ ID NOs: 351 and 362; SEQ ID NOs: 351 and 407; SEQ ID NOs: 351 and 408; SEQ ID NOs: 351 and 409; SEQ ID NOs: 351 and 410; SEQ ID NOs: 352 and 357; SEQ ID NOs: 352 and 358; SEQ ID NOs: 352 and 359; SEQ ID NOs: 352 and 360; SEQ ID NOs: 352 and 361; SEQ ID NOs: 352 and 362; SEQ ID NOs: 352 and 407; SEQ ID NOs: 352 and 408; SEQ ID NOs: 352 and 409; SEQ ID NOs: 352 and 410; SEQ ID NOs: 430 and 357; SEQ ID NOs: 430 and 358; SEQ ID NOs: 430 and 359; SEQ ID NOs: 430 and 360; SEQ ID NOs: 430 and 361; SEQ ID NOs: 430 and 362; SEQ ID NOs: 407 and 409; SEQ ID NOs: 407 and 408; SEQ ID NOs: 408 and 410; or SEQ ID NOs: 409 and 410.

Embodiment 123: The immunogenic composition of any one of embodiments 117 to 122, comprising:
1) One or more fusion polypeptides comprising an amino acid sequence of any one of SEQ ID NOs: 351-356 and 430, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 351-356 and 430; and
2) One or more fusion polypeptides comprising an amino acid sequence of any one of SEQ ID NOs: 357-366 and 407-410, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 357-366 and 407-410.

Embodiment 124: A pharmaceutical composition comprising one or more of the fusion polypeptides of any one of embodiments 1 to 89, or one or more polynucleotides of any one of embodiments 90 to 93, or one or more vectors of any one of embodiments 98 to 110, and a pharmaceutically acceptable carrier.

Embodiment 125: The pharmaceutical composition of embodiment 124, comprising two or more fusion polypeptides, two or more polynucleotides or two or more vectors.

Embodiment 126: The pharmaceutical composition of any one of embodiments 124 to 125, further comprising one or more of an adjuvant, an immunostimulator, a detergent, a micelle-forming agent, and an oil.

Embodiment 127: The pharmaceutical composition of embodiment 126, wherein the immunomodulator is selected from a toll-like receptor (TLR) agonist, a cytokine (e.g., IL-2, IL-7, IL-12, IL-15, IL-18, IL-21, IFN-α, IFN-γ, GM-CSF, FLT3LG, and combinations and functional variants thereof), a non-coding immunostimulatory polynucleotide (e.g., a pathogen-activated molecular pattern (PAMP), a cytosine-phosphate-guanosine (CpG) oligodeoxynucleotide, and an immunostimulatory RNA (isRNA, e.g., CV8102)), an inhibitor of an inhibitory immune checkpoint protein or a stimulator of a stimulatory immune checkpoint protein.

Embodiment 128: The pharmaceutical composition of any one of embodiments 124 to 127, formulated for administration via a route selected from the group consisting of intravenous, intramuscular, intradermal, subcutaneous and mucosal (e.g. buccal, intranasal, intrarectal, intravaginal).

Embodiment 129: The pharmaceutical composition of any one of embodiments 124 to 128, formulated as a liquid.

Embodiment 130: The pharmaceutical composition of any one of embodiments 124 to 128, wherein the composition is lyophilized.

### Kits

Embodiment 131: A kit comprising one or more unitary doses of one or more of the fusion polypeptides of any one of embodiments 1 to 89, or one or more polynucleotides of any one of embodiments 90 to 93, or one or more vectors of any one of embodiments 98 to 110, or one or more immunogenic compositions of any one of embodiments 117 to 121, or one or more pharmaceutical compositions of any one of embodiments 124 to 130.

Embodiment 132: The kit of embodiment 131, wherein the one or more unitary doses are in a single container.

Embodiment 133: The kit of embodiment 131, wherein the one or more unitary doses are in two or more separate containers.

Embodiment 134: The kit of any one of embodiments 131 to 133, comprising one or more containers selected from the group consisting of vials, ampules and pre-loaded syringes.

Embodiment 135: The kit of any one of embodiments 131 to 134, comprising one or more containers comprising the one or more fusion polypeptides, one or more polynucleotides or one or more vectors in an aqueous solution.

Embodiment 136: The kit of any one of embodiments 131 to 135, wherein the one or more unitary doses are the same.

Embodiment 137: The kit of any one of embodiments 131 to 135, wherein the one or more unitary doses are the different.

Embodiment 138: The kit of any one of embodiments 131 to 137, comprising one or more unitary doses of one or more viral vectors of any one of embodiments 98 to 110, wherein the unitary doses are in the range of about 10³ to about 10¹⁵ viral focus forming units (FFU) or plaque forming units (PFU) or infectious units (IU) or viral particles (vp), e.g. from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp.

Embodiment 139: The kit of any one of embodiments 131 to 138, comprising two or more of the fusion polypeptides of any one of embodiments 1 to 89, or two or more polynucleotides of any one of embodiments 90 to 93, or two or more vectors of any one of embodiments 98 to 110.

Embodiment 140: The kit of embodiment 139, comprising two or more polynucleotides encoding or two or more vectors expressing the fusion polypeptides, the fusion polypeptides comprising:
1) One or more fusion polypeptides comprising or consisting of the following polypeptide segments in sequential order, from N-terminus to C-terminus, optionally joined or connected by one or more linkers:
SEQ ID NOs: 70, 76, 94, 151 and 161; or
SEQ ID NOs: 71, 77, 95, 152 and 162; and
2) One or more fusion polypeptides comprising or consisting of the following polypeptide segments in sequential order, from N-terminus to C-terminus, optionally joined or connected by one or more linkers:
SEQ ID NOs: 188, 305, 28, 41, 294, 4, 176, 11, 319, 259, 282, 223, 213 and 37;
SEQ ID NOs: 188, 305, 28, 41 and 294;
SEQ ID NOs: 4, 176, 11, 319, 259, 282, 223, 213 and 37;
SEQ ID NOs: 189, 306, 29, 42, 295, 5, 177, 12, 320, 260, 283, 224, 214, and 38;
SEQ ID NOs: 189, 306, 29, 42 and 295;
SEQ ID NOs: 5, 177, 12, 320, 260, 283, 224, 214 and 38;
SEQ ID NOs: 305, 319, 259, 282, 223, 213, 294, 176 and 188;
SEQ ID NOs: 306, 320, 260, 283, 224, 214, 295, 177 and 189;
SEQ ID NOs: 305, 294, 223, 213, 176, 259, 319, 188 and 282;
SEQ ID NOs: 306, 295, 224, 214, 177, 260, 320, 189 and 283;
SEQ ID NOs: 305, 294, 319, 259, 282, 223, 176, and 188;
SEQ ID NOs: 306, 295, 320, 260, 283, 224, 177 and 189;
SEQ ID NOs: 305, 223, 294, 176, 259, 319, 188 and 282; or
SEQ ID NOs: 306, 224, 295, 177, 260, 320, 189 and 283.

Embodiment 141: The kit of embodiment 139, comprising two or more polynucleotides encoding or two or more vectors expressing the fusion polypeptides, the fusion polypeptides comprising:
1) One or more fusion polypeptides comprising, one or more polynucleotides encoding or one or more vectors capable of expressing, an amino acid sequence of any one of SEQ ID NOs: 351-356 and 430, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 351-356 and 430; and
2) One or more fusion polypeptides comprising, one or more polynucleotides encoding or one or more vectors capable of expressing, an amino acid sequence of any one of SEQ ID NOs: 357-366 and 407-410, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 357-366 and 407-410.

Embodiment 142: The kit of any one of embodiments 131 to 141, comprising one or more polynucleotides encoding or one or more vectors expressing the fusion polypeptides, the fusion polypeptides comprising or consisting of the following polypeptide segments in sequential order, from N-terminus to C-terminus, optionally joined or connected by one or more linkers:
SEQ ID NOs: 201, 78, 107, 96, 229, 172, 327, 6, 333, 243, 331, 192, 265, 311, 137, 15, 123, 30, 336, 302, 153, 219, 298, 121, 230, 240, 60, 241, 276, 113, 99, 21, 217 and 215;
SEQ ID NOs: 78, 296, 1, 339, 197, 329, 232, 323, 303, 234, 90, 261, 274, 238, 211, 325, 137, 227, 209, 190, 341, 57,225, 27, 210, 119, 19, 165, 334, 117, 153, 10, 97 and 300; or
SEQ ID NOs: 296, 1, 78, 197, 339, 227, 261, 274, 238, 325, 137, 329, 303, 234, 90, 232, 27, 57, 225, 323, 190, 341, 119, 19, 165, 334, 117, 153, 10, 97 and 300.

Embodiment 143: The kit of any one of embodiments 131 to 142, comprising one or more polynucleotides encoding or one or more vectors expressing the fusion polypeptides, the fusion polypeptides comprising or consisting of an amino acid sequence of any one of SEQ ID NOs: 345-377, 411, 422-424 and 430-435, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 345-377, 411, 422-424 and 430-435.

Embodiment 144: The kit of any one of embodiments 131 to 143, comprising one or more adenoviral vectors, each adenoviral vector comprising one or more polynucleotides encoding one or more fusion proteins comprising an amino acid sequence of any one of any one of SEQ ID NOs: 345-377, 407-411, 422-424, 430-435, or that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 345-377, 407-411, 422-424, 430-435.

Embodiment 145: The kit of any one of embodiments 131 to 144, comprising one or more viral vectors, wherein each viral vector comprises two or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the following amino acid sequences: SEQ ID NOs: 345 and 346; SEQ ID NOs: 347 and 348; SEQ ID NOs: 349 and 350; SEQ ID NOs: 351 and 352; SEQ ID NOs: 430 and 352; SEQ ID NOs: 357 and 358; SEQ ID NOs: 360 and 362; SEQ ID NOs: 359 and 361; SEQ ID NOs: 351 and 357; SEQ ID NOs: 351 and 358; SEQ ID NOs: 351 and 359; SEQ ID NOs: 351 and 360; SEQ ID NOs: 351 and 361; SEQ ID NOs: 351 and 362; SEQ ID NOs: 351 and 407; SEQ ID NOs: 351 and 408; SEQ ID NOs: 351 and 409; SEQ ID NOs: 351 and 410; SEQ ID NOs: 352 and 357; SEQ ID NOs: 352 and 358; SEQ ID NOs: 352 and 359; SEQ ID NOs: 352 and 360; SEQ ID NOs: 352 and 361; SEQ ID NOs: 352 and 362; SEQ ID NOs: 352 and 407; SEQ ID NOs: 352 and 408; SEQ ID NOs: 352 and 409; SEQ ID NOs: 352 and 410; SEQ ID NOs: 430 and 357; SEQ ID NOs: 430 and 358; SEQ ID NOs: 430 and 359; SEQ ID NOs: 430 and 360; SEQ ID NOs: 430 and 361; SEQ ID NOs: 430 and 362; SEQ ID NOs: 407 and 409; SEQ ID NOs: 407 and 408; SEQ ID NOs: 408 and 410; or SEQ ID NOs: 409 and 410.

Embodiment 146: The kit of any one of embodiments 131 to 145, further comprising one or more unitary doses of one or more additional therapeutic agents.

Embodiment 147: The kit of embodiment 146, comprising one or more agents that activate latent HIV, e.g., one or more latency reversing agents (LRAs).

Embodiment 148: The kit of any one of embodiments 146 to 147, comprising one or more LRAs selected from the group consisting of agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics.

Embodiment 149: The kit of any one of embodiments 146 to 148, comprising one or more agonists or activators of one or more toll-like receptors (TLRs).

Embodiment 150: The kit of embodiment 149, wherein the TLR agonist or activator is selected from the group consisting of a TLR2 agonist, a TLR3 agonist, a TLR4 agonist, a TLR5 agonist, a TLR7 agonist, a TLR8 agonist and a TLR9 agonist.

Embodiment 151: The kit of any one of embodiments 149 to 150, wherein the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist).

Embodiment 152: The kit of any one of embodiments 149 to 151, wherein the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042.

Embodiment 153: The kit of any one of embodiments 146 to 152, comprising one or more interleukin receptor agonists of an interleukin selected from IL-2, IL-7, IL-12, IL-15, IL-18, IL-21, IFN-α, IFN-γ, GM-CSF and FLT3LG.

Embodiment 154: The kit of embodiment 153, comprising one or more cytokines selected from the group consisting of IL-2, IL-7, IL-12, IL-15, IL-18, IL-21, IFN-α, IFN-γ, GM-CSF, FLT3LG, and combinations and functional variants thereof.

Embodiment 155: The kit of any one of embodiments 146 to 154, comprising one or more innate immune activators.

Embodiment 156: The kit of embodiment 155, wherein the one or more innate immune activators comprises a non-coding immunostimulatory polynucleotide (e.g., a pathogen-activated molecular pattern (PAMP), a cytosine-phosphate-guanosine (CpG) oligodeoxynucleotide, and an immunostimulatory RNA (isRNA, e.g., CV8102)), an agonist of a receptor selected from the group consisting of fins related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2).

Embodiment 157: The kit of any one of embodiments 146 to 156, comprising one or more blockers, antagonists or inhibitors of an inhibitory immune checkpoint protein or receptor and/or one or more activators or agonists of a stimulatory immune checkpoint protein or receptor.

Embodiment 158: The kit of embodiment 157, wherein the one or more immune checkpoint proteins or receptors are selected from the group consisting of: CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); and SLAM family member 7 (SLAMF7).

Embodiment 159: The kit of any one of embodiments 157 to 158, comprising one or more blockers, antagonists or inhibitors of one or more T-cell inhibitory immune checkpoint proteins or receptors.

Embodiment 160: The kit of embodiment 159, wherein the T-cell inhibitory immune checkpoint proteins or receptors are selected from the group consisting of CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1).

Embodiment 161: The kit of any one of embodiments 157 to 160, comprising one or more agonists or activators of one or more T-cell stimulatory immune checkpoint proteins or receptors.

Embodiment 162: The kit of embodiment 161, wherein the T-cell stimulatory immune checkpoint proteins or receptors are selected from the group consisting of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155).

Embodiment 163: The kit of any one of embodiments 157 to 162, comprising one or more blockers, antagonists or inhibitors of one or more NK-cell inhibitory immune checkpoint proteins or receptors.

Embodiment 164: The kit embodiment 163, wherein the NK-cell inhibitory immune checkpoint proteins or receptors are selected from the group consisting of killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94).

Embodiment 165: The kit of any one of embodiments 157 to 164, comprising one or more agonists or activators of one or more NK-cell stimulatory immune checkpoint proteins or receptors.

Embodiment 166: The kit of embodiment 165, wherein the NK-cell stimulatory immune checkpoint proteins or receptors are selected from CD16, CD226 (DNAM-1); killer cell lectin like receptor K1 (KLRK1, NKG2D, CD314); and SLAM family member 7 (SLAMF7).

Embodiment 167: The kit of any one of embodiments 157 to 166, wherein the one or more immune checkpoint inhibitors comprises a proteinaceous inhibitor of PD-L1 (CD274), PD-1 (PDCD1) or CTLA4.

Embodiment 168: The kit of embodiment 167, wherein the proteinaceous inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4) and AK-104 (CTLA4/PD-1).

Embodiment 169: The kit of embodiment 167, wherein the proteinaceous inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

Embodiment 170: The kit of any one of embodiments 157 to 169, wherein the one or more immune checkpoint inhibitors comprises a small molecule inhibitor of CD274 (PDL1, PD-L1), programmed cell death 1 (PDCD1, PD1, PD-1) or CTLA4.

Embodiment 171: The kit of embodiment 170, wherein the small molecule inhibitor of CD274 or PDCD1 is selected from the group consisting of GS-4224, GS-4416, INCB086550 and MAX10181.

Embodiment 172: The kit of embodiment 170, wherein the small molecule inhibitor of CTLA4 comprises BPI-002.

Embodiment 173: The kit of any one of embodiments 146 to 172, further comprising one or more anti-viral agents.

Embodiment 174: The kit of embodiment 173, wherein the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors.

### Methods of Treating or Preventing HIV

Embodiment 175: A method for eliciting an immune response to human immunodeficiency virus (HIV) in a subject in need thereof, comprising administering to the subject the pharmaceutical composition of any one of embodiments 124 to 130, or the immunogenic composition of any one of embodiments 117 to 121.

Embodiment 176: A method of treating or preventing human immunodeficiency virus (HIV) in a subject in need thereof, comprising administering to the subject the pharmaceutical composition of any one of embodiments 124 to 130, the immunogenic composition of any one of embodiments 117 to 121.

Embodiment 177: The method of any one of embodiments 175 to 176, comprising administering a single fusion polypeptide, or a polynucleotide or viral expression vector encoding the fusion polypeptide, wherein the fusion polypeptide comprises two or more multivalent polypeptide segments, e.g., bivalent polypeptide segments.

Embodiment 178: The method of any one of embodiments 175 to 176, wherein two or more fusion polypeptides, or two or more viral expression vectors encoding the fusion polypeptides, are administered to the subject simultaneously or concurrently.

Embodiment 179: The method of any one of embodiments 175 to 178, wherein two or more fusion polypeptides, or two or more polynucleotides or two or more viral expression vectors encoding the fusion polypeptides, are in the form of a bivalent antigen composition.

Embodiment 180: The method of any one of embodiments 175 to 179, comprising administering to the subject:
1) one or more fusion polypeptides, or polynucleotides encoding, or viral expression vectors expressing the fusion polypeptides, the fusion polypeptides comprising or consisting of the following polypeptide segments in sequential order, from N-terminus to C-terminus, optionally joined or connected by one or more linkers:
SEQ ID NOs: 70, 76, 94, 151 and 161; or
SEQ ID NOs: 71, 77, 95, 152 and 162; and
2) one or more fusion polypeptides, or polynucleotides encoding, or viral expression vectors expressing the fusion polypeptides, the fusion polypeptides comprising or consisting of the following polypeptide segments in sequential order, from N-terminus to C-terminus, optionally joined or connected by one or more linkers:
SEQ ID NOs: 188, 305, 28, 41, 294, 4, 176, 11, 319, 259, 282, 223, 213 and 37;
SEQ ID NOs: 188, 305, 28, 41 and 294;
SEQ ID NOs: 4, 176, 11, 319, 259, 282, 223, 213 and 37;
SEQ ID NOs: 189, 306, 29, 42, 295, 5, 177, 12, 320, 260, 283, 224, 214 and 38;
SEQ ID NOs: 189, 306, 29, 42 and 295;
SEQ ID NOs: 5, 177, 12, 320, 260, 283, 224, 214 and 38;
SEQ ID NOs: 305, 319, 259, 282, 223, 213, 294, 176 and 188;
SEQ ID NOs: 306, 320, 260, 283, 224, 214, 295, 177 and 189;
SEQ ID NOs: 305, 294, 223, 213, 176, 259, 319, 188 and 282;
SEQ ID NOs: 306, 295, 224, 214, 177, 260, 320, 189 and 283;
SEQ ID NOs: 305, 294, 319, 259, 282, 223, 176, and 188;
SEQ ID NOs: 306, 295, 320, 260, 283, 224, 177 and 189;
SEQ ID NOs: 305, 223, 294, 176, 259, 319, 188 and 282; or
SEQ ID NOs: 306, 224, 295, 177, 260, 320, 189 and 283.

Embodiment 181: The method of any one of embodiments 175 to 180, comprising administering to the subject:
1) one or more fusion polypeptides, or polynucleotides encoding, or viral expression vectors expressing the fusion polypeptides, the fusion polypeptides comprising or consisting of an amino acid sequence of any one of SEQ ID NOs: 351-356 and 430, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 351-356 and 430; and
2) one or more fusion polypeptides, or polynucleotides encoding, or viral expression vectors expressing the fusion polypeptides, the fusion polypeptides comprising or consisting of an amino acid sequence of any one of SEQ ID NOs: 357-366 and 407-410, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 357-366 and 407-410.

Embodiment 182: The method of any one of embodiments 175 to 178, comprising administering to the subject one or more fusion polypeptides, or polynucleotides encoding, or viral expression vectors expressing the fusion polypeptides, the fusion polypeptides comprising or consisting of the following polypeptide segments in sequential order, from N-terminus to C-terminus, optionally joined or connected by one or more linkers:
SEQ ID NOs: 201, 78, 107, 96, 229, 172, 327, 6, 333, 243, 331, 192, 265, 311, 137, 15, 123, 30, 336, 302, 153, 219, 298, 121, 230, 240, 60, 241, 276, 113, 99, 21, 217 and 215;
SEQ ID NOs: 78, 296, 1, 339, 197, 329, 232, 323, 303, 234, 90, 261, 274, 238, 211, 325, 137, 227, 209, 190, 341, 57, 225, 27, 210, 119, 19, 165, 334, 117, 153, 10, 97 and 300; or
SEQ ID NOs: 296, 1, 78, 197, 339, 227, 261, 274, 238, 325, 137, 329, 303, 234, 90, 232, 27, 57, 225, 323, 190, 341, 119, 19, 165, 334, 117, 153, 10, 97 and 300.

Embodiment 183: The method of any one of embodiments 175 to 182, comprising administering to the subject one or more fusion polypeptides, or polynucleotides encoding, or viral expression vectors expressing the fusion polypeptides, the fusion polypeptides comprising or consisting of an amino acid sequence of any one of SEQ ID NOs: 345-377, 407-411, 422-424, 430-435, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 345-377, 407-411, 422-424, 430-435.

Embodiment 184: The method of any one of embodiments 175 to 183, comprising administering to the subject one or more adenoviral vectors, each adenoviral vector comprising one or more polynucleotides encoding one or more fusion proteins comprising an amino acid sequence of any one of any one of SEQ ID NOs: 345-377, 407-411, 422-424, 430-435, or that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 345-377, 407-411, 422-424, 430-435.

Embodiment 185: The method of any one of embodiments 175 to 183, comprising administering to the subject one or more viral vectors, wherein each viral vector comprises two or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the following amino acid sequences: SEQ ID NOs: 345 and 346; SEQ ID NOs: 347 and 348; SEQ ID NOs: 349 and 350; SEQ ID NOs: 351 and 352; SEQ ID NOs: 430 and 352; SEQ ID NOs: 357 and 358; SEQ ID NOs: 360 and 362; SEQ ID NOs: 359 and 361; SEQ ID NOs: 351 and 357; SEQ ID NOs: 351 and 358; SEQ ID NOs: 351 and 359; SEQ ID NOs: 351 and 360; SEQ ID NOs: 351 and 361; SEQ ID NOs: 351 and 362; SEQ ID NOs: 351 and 407; SEQ ID NOs: 351 and 408; SEQ ID NOs: 351 and 409; SEQ ID NOs: 351 and 410; SEQ ID NOs: 352 and 357; SEQ ID NOs: 352 and 358; SEQ ID NOs: 352 and 359; SEQ ID NOs: 352 and 360; SEQ ID NOs: 352 and 361; SEQ ID NOs: 352 and 362; SEQ ID NOs: 352 and 407; SEQ ID NOs: 352 and 408; SEQ ID NOs: 352 and 409; SEQ ID NOs: 352 and 410; SEQ ID NOs: 430 and 357; SEQ ID NOs: 430 and 358; SEQ ID NOs: 430 and 359; SEQ ID NOs: 430 and 360; SEQ ID NOs: 430 and 361; SEQ ID NOs: 430 and 362; SEQ ID NOs: 407 and 409; SEQ ID NOs: 407 and 408; SEQ ID NOs: 408 and 410; or SEQ ID NOs: 409 and 410.

Embodiment 186: The method of any one of embodiments 175 to 185, wherein the subject is infected with HIV-1, is suspected of being infected with HIV-1, or is at risk of being infected with HIV-1.

Embodiment 187: The method of any one of embodiments 175 to 186, wherein the subject is chronically infected with HIV-1.

Embodiment 188: The method of any one of embodiments 175 to 187, wherein the subject is acutely infected with HIV-1.

Embodiment 189: The method of any one of embodiments 175 to 188, wherein the subject has an HIV-1 infection of Fiebig stage IV or earlier, e.g. Fiebig stage II, Fiebig stage II or Fiebig stage I.

Embodiment 190: The method of any one of embodiments 175 to 189, wherein the composition is administered via a route selected from intravenous, intramuscular, intradermal, subcutaneous and mucosal (e.g. buccal, intranasal, intrarectal, intravaginal).

Embodiment 191: The method of any one of embodiments 175 to 190, comprising administering from about 10³ to about 10¹⁵ viral focus forming units (FFU) or plaque forming units (PFU) or infectious units (IU) or viral particles (vp), e.g. from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration.

Embodiment 192: The method of any one of embodiments 175 to 191, comprising a prime-boost regimen comprising:
(i) administering a priming composition at a first time point and administering one or more boosting compositions at one or more subsequent time points (e.g., prime-boost-boost-boost, etc.); or
(ii) one or more iterations of administering a priming composition at a first time point and administering a boosting composition at a second time point (e.g., prime-boost-prime-boost, etc.).

Embodiment 193: The method of embodiment 192, wherein the administrations of the priming composition and the one or more boosting compositions are spaced at least 1 week, 2 weeks, 3 weeks or 1 month apart, e.g., at least 2, 3, 4, 5 or 6 months, apart.

Embodiment 194: The method of any one of embodiments 192 to 193, wherein the priming composition and the boosting composition comprise the same immunogenic composition.

Embodiment 195: The method of any one of embodiments 192 to 193, wherein the priming composition and the boosting composition comprise different immunogenic compositions.

Embodiment 196: The method of any one of embodiments 192 to 193, wherein the priming composition and the boosting composition comprise the same one or more fusion polypeptides and same polynucleotide or viral expression vector.

Embodiment 197: The method of any one of embodiments 192 to 193, wherein the priming composition and the boosting composition comprise different fusion polypeptides and/or different polynucleotide or viral expression vectors.

Embodiment 198: The method of embodiment 197, comprising priming with a first polynucleotide or viral expression vector, and boosting with a second polynucleotide or viral expression vector.

Embodiment 199: The method of any one of embodiments 192 to 198, wherein the prime-boost regimen comprises:
a) Priming with a viral expression vector and boosting with a polynucleotide, wherein the polynucleotide is DNA, cDNA, mRNA or self-replicating RNA;
b) Priming with a polynucleotide, wherein the polynucleotide is DNA, cDNA, mRNA or self-replicating RNA, and boosting with a viral expression vector;
c) Priming with a first viral expression vector and boosting with a second viral expression vector, wherein the first and second viral expression vectors are from identical, related or unrelated taxonomical families;
d) Priming with a first replication deficient viral expression vector and boosting with a second replication deficient viral expression vector, wherein the first and second replication deficient viral expression vectors are from identical, related or unrelated taxonomical families;
e) Priming with a first attenuated deficient viral expression vector and boosting with a second replication attenuated viral expression vector, wherein the first and second replication attenuated viral expression vectors are from identical, related or unrelated taxonomical families;
f) Priming with a replication deficient viral expression vector and boosting with a replication attenuated viral expression vector;
g) Priming with a replication attenuated viral expression vector and boosting with a replication deficient viral expression vector;
h) Priming with a Lymphocytic choriomeningitis mammarenavirus (LCMV) viral expression vector and boosting with a Pichinde mammarenavirus viral expression vector;
i) Priming with a Pichinde mammarenavirus viral expression vector and boosting with a Lymphocytic choriomeningitis mammarenavirus (LCMV) viral expression vector;
j) Priming with an arenavirus viral expression vector and boosting with an adenovirus viral expression vector; or
k) Priming with an adenovirus viral expression vector and boosting with an arenavirus viral expression vector.

Embodiment 200: The method of any one of embodiments 175 to 199, wherein the subject is not receiving antiretroviral therapy (ART) or ART is discontinued prior to administration of the one or more compositions.

Embodiment 201: The method of any one of embodiments 175 to 200, wherein ART is discontinued after one or more administrations of the compositions.

Embodiment 202: The method of any one of embodiments 175 to 201, further comprising administering to the subject one or more additional therapeutic agents, e.g. two, three, four, or more additional therapeutic agents.

Embodiment 203: The method of embodiment 202, comprising co-administering one or more agents that activate latent HIV, e.g., one or more latency reversing agents (LRAs).

Embodiment 204: The method of any one of embodiments 202 to 203, wherein the one or more LRAs are selected from the group consisting of agonists or activators of one or more toll-like receptors (TLRs), histone deacetylase (HDAC) inhibitors, proteasome inhibitors, protein kinase C (PKC) activators, Smyd2 inhibitors, BET-bromodomain 4 (BRD4) inhibitors, ionomycin, inhibitor of apoptosis proteins (IAP) antagonists, and second mitochondria-derived activator of caspases (SMAC) mimetics.

Embodiment 205: The method of any one of embodiments 202 to 204, comprising co-administering one or more agonists or activators of one or more toll-like receptors (TLRs).

Embodiment 206: The method of embodiment 205, wherein the TLR agonist or activator is selected from the group consisting of a TLR2 agonist, a TLR3 agonist, a TLR4 agonist, a TLR5 agonist, a TLR7 agonist, a TLR8 agonist and a TLR9 agonist.

Embodiment 207: The method of any one of embodiments 205 to 206, wherein the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist).

Embodiment 208: The method of any one of embodiments 202 to 207, comprising co-administering one or more interleukin receptor agonists of an interleukin selected from IL-2, IL-7, IL-12, IL-15, IL-18, IL-21, IFN-α, IFN-γ, GM-CSF and FLT3LG.

Embodiment 209: The method of embodiment 208, comprising co-administering one or more cytokines selected from the group consisting of IL-2, IL-7, IL-12, IL-15, IL-18, IL-21, IFN-α, IFN-γ, GM-CSF, FLT3LG, and combinations and functional variants thereof.

Embodiment 210: The method of any one of embodiments 202 to 209, comprising co-administering one or more innate immune activators.

Embodiment 211: The method of embodiment 210, wherein the one or more innate immune activators comprises a non-coding immunostimulatory polynucleotide (e.g., a pathogen-activated molecular pattern (PAMP), a cytosine-phosphate-guanosine (CpG) oligodeoxynucleotide, and an immunostimulatory RNA (isRNA, e.g., CV8102)), an agonist of a receptor selected from the group consisting of fins related tyrosine kinase 3 (FLT3), stimulator of interferon genes (STING) receptor, DExD/H-box helicase 58 (DDX58; a.k.a., RIG-I), nucleotide binding oligomerization domain containing 2 (NOD2).

Embodiment 212: The method of any one of embodiments 202 to 211, comprising co-administering one or more antagonists or inhibitors of an inhibitory immune checkpoint protein or receptor and/or one or more activators or agonists of a stimulatory immune checkpoint protein or receptor.

Embodiment 213: The method of embodiment 212, wherein the one or more immune checkpoint proteins or receptors are selected from the group consisting of: CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); and SLAM family member 7 (SLAMF7).

Embodiment 214: The method of any one of embodiments 212 of 213, comprising co-administering one or more blockers, antagonists or inhibitors of one or more T-cell inhibitory immune checkpoint proteins or receptors.

Embodiment 215: The method of embodiment 214, wherein the T-cell inhibitory immune checkpoint proteins or receptors are selected from the group consisting of CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1).

Embodiment 216: The method of any one of embodiments 212 of 213, comprising co-administering one or more agonists or activators of one or more T-cell stimulatory immune checkpoint proteins or receptors.

Embodiment 217: The method of embodiment 216, wherein the T-cell stimulatory immune checkpoint proteins or receptors are selected from the group consisting of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155).

Embodiment 218: The method of any one of embodiments 212 of 213, comprising co-administering one or more blockers, antagonists or inhibitors of one or more NK-cell inhibitory immune checkpoint proteins or receptors.

Embodiment 219: The method of embodiment 218, wherein the NK-cell inhibitory immune checkpoint proteins or receptors are selected from the group consisting of killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94).

Embodiment 220: The method of any one of embodiments 212 of 213, comprising co-administering one or more agonists or activators of one or more NK-cell stimulatory immune checkpoint proteins or receptors.

Embodiment 221: The method of embodiment 220, wherein the NK-cell stimulatory immune checkpoint proteins or receptors are selected from CD16, CD226 (DNAM-1); killer cell lectin like receptor K1 (KLRK1, NKG2D, CD314); and SLAM family member 7 (SLAMF7).

Embodiment 222: The method of any one of embodiments 212 to 215, wherein the one or more immune checkpoint inhibitors comprises a proteinaceous inhibitor of PD-L1 (CD274), PD-1 (PDCD1) or CTLA4.

Embodiment 223: The method of embodiment 222, wherein the proteinaceous inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4) and AK-104 (CTLA4/PD-1).

Embodiment 224: The method of embodiment 222, wherein the proteinaceous inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

Embodiment 225: The method of any one of embodiments 212 to 215, wherein the one or more immune checkpoint inhibitors comprises a small molecule inhibitor of CD274 (PDL1, PD-L1), programmed cell death 1 (PDCD1, PD1, PD-1) or CTLA4.

Embodiment 226: The method of embodiment 225, wherein the small molecule inhibitor of CD274 or PDCD1 is selected from the group consisting of GS-4224, GS-4416, INCB086550 and MAX10181.

Embodiment 227: The method of embodiment 225, wherein the small molecule inhibitor of CTLA4 comprises BPI-002.

Embodiment 228: The method of any one of embodiments 202 to 227, further comprising administering to the subject one or more anti-viral agents.

Embodiment 229: The method of 228, wherein the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors and capsid inhibitors.

Embodiment 230: The method of any one of embodiments 202 to 229, further comprising administering to the subject one or more anti-HIV antibodies or antigen-binding fragments thereof.

Embodiment 231: The method of embodiment 230, wherein the one or more anti-HIV antibodies or antigen-binding fragments thereof binds to HIV gp120.

Embodiment 232: The method of any one of embodiments 230 to 231, wherein the anti-HIV antibody or antigen-binding fragment thereof comprises a broadly neutralizing antibody.

Embodiment 233: The method of any one of embodiments 230 to 232, wherein one or more anti-HIV antibodies or antigen-binding fragments thereof that bind, inhibit, and/or neutralize HIV, compete with or comprise VH and VL variable domains of a broadly neutralizing antibody (bNAb) against HIV.

Embodiment 234: The method of any one of embodiments 230 to 233, wherein one or more anti-HIV antibodies or antigen-binding fragments thereof that bind, inhibit, and/or neutralize HIV, bind to an epitope or region of gp120 selected from the group consisting of:
i. third variable loop (V3) and/or high mannose patch comprising a N332 oligomannose glycan;
ii. CD4 binding site (CD4bs);
iii. second variable loop (V2) and/or Env trimer apex;
iv. gp120/gp41 interface; or
v. silent face of gp120.

Embodiment 235: The method of any one of embodiments 230 to 234, wherein the antibody or antigen-binding fragment thereof that binds, inhibits, and/or neutralizes HIV, binds to an epitope or region of gp120 in the third variable loop (V3) and/or high mannose patch comprising a N332 oligomannose glycan and competes with or comprises VH and VL regions from an antibody selected from the group consisting of GS-9722, PGT-121, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03.

Embodiment 236: The method of any one of embodiments 230 to 235, wherein the antibody or antigen-binding fragment thereof binds to an epitope or region of gp120 in the CD4 binding site (CD4bs) and competes with or comprises VH and VL regions from an antibody selected from the group consisting of b12, F105, VRC01, VRC07, VRC07-523, VRC03, VRC06, VRC06b01 VRC08, VRC0801, NIH45-46, GS-9723, 3BNC117, 3BNC60, VRC-PG04, PGV04; CH103, 44-VRC13.01, 1NC9, 12A12, N6, N49-P7, NC-Cow1, IOMA, CH235 and CH235.12, N49P6, N49P7, N49P11, N49P9 and N60P25.

Embodiment 237: The method of any one of embodiments 230 to 236, wherein the antibody or antigen-binding fragment thereof that binds, inhibits, and/or neutralizes HIV, binds to an epitope or region of gp120 in the second variable loop (V2) and/or Env trimer apex and competes with or comprises VH and VL regions from an antibody selected from the group consisting of PG9, PG16, PGC14, PGG14, PGT-142, PGT-143, PGT-144, PGT-145, CH01, CH59, PGDM1400, CAP256, CAP256-VRC26.08, CAP256-VRC26.09, CAP256-VRC26.25, PCT64-24E and VRC38.01.

Embodiment 238: The method of any one of embodiments 230 to 237, wherein the antibody or antigen-binding fragment binds to an epitope or region of gp120 in the gp120/gp41 interface and competes with or comprises VH and VL regions from an antibody selected from the group consisting of PGT-151, CAP248-2B, 35022, 8ANC195, ACS202, VRC34 and VRC34.01.

Embodiment 239: The method of any one of embodiments 230 to 238, wherein the antibody or antigen-binding fragment thereof that binds, inhibits, and/or neutralizes HIV, binds to an epitope or region of the gp120 silent face and competes with or comprises VH and VL regions from antibody selected from the group consisting of VRC-PG05 and SF12.

Embodiment 240: The method of any one of embodiments 230 to 239, wherein the antibody or antigen-binding fragment thereof that binds, inhibits, and/or neutralizes HIV, binds to an epitope or region of gp41 in the membrane proximal region (MPER).

Embodiment 241: The method of any one of embodiments 230 to 240, wherein the antibody or antigen-binding fragment thereof that binds, inhibits, and/or neutralizes HIV, binds to an epitope or region of gp41 in the membrane proximal region (MPER) and competes with or comprises VH and VL regions from an antibody selected from the group consisting of 10E8, 10E8v4, 10E8-5R-100cF, 4E10, DH511.11P, 2F5, 7b2, and LN01.

Embodiment 242: The method of any one of embodiments 230 to 241, wherein the antibody or antigen-binding fragment thereof that binds, inhibits, and/or neutralizes HIV, binds to an epitope or region of the gp41 fusion peptide and competes with or comprises VH and VL regions from an antibody selected from the group consisting of VRC34 and ACS202.

Embodiment 243: The method of any one of embodiments 175 to 242, wherein, after one or more administrations of one or more of the compositions, optionally in combination with one or more additional therapeutic agents, the subject does not exhibit symptoms of HIV or AIDS in the absence of anti-retroviral treatment (ART) for at least 6 months, at least 1 year, at least 2 years, at least 3 years, or more.

Embodiment 244: The method of any one of embodiments 175 to 243, wherein, after one or more administrations of one or more of the compositions, optionally in combination with one or more additional therapeutic agents, the subject has a viral load copies/ml blood of less than 500, e.g. less than 400, less than 300, less than 200, less than 100, less than 50, in the absence of anti-retroviral treatment (ART) for at least 6 months, at least 1 year, at least 2 years, at least 3 years, or more.

### Methods of Immunogen Design

Embodiment 245: A method of designing a fusion polypeptide that is capable of eliciting an immune response against one or more viral target antigens, the method comprising:
a) identifying in silico one or more regions of sequence conservation in a population of polypeptide sequences encoded by a viral gene, the population from an interpatient virus population; and
b) identifying in silico the two most prevalent polypeptide sequences from the one or more conserved regions identified in step a), and generating multivalent polypeptide segments from the conserved regions.

Embodiment 246: The method of embodiment 245, wherein the multivalent polypeptide segments are bivalent polypeptide segments.

Embodiment 247: The method of any one of embodiments 245 to 246, further comprising step c): arranging the polypeptide segments into one or more contiguous fusion polypeptides, such that the junctions connecting the polypeptide segments reduce or avoid creating epitopes capable of binding human MHC class I or human MHC class II molecules, e.g., with a predicted binding affinity IC50 value of less than about 1000 nM or having a percentile rank within the top 5% in a population of polypeptide segments.

Embodiment 248: The method of any one of embodiments 245 to 247, further comprising the step of inserting a linker between polypeptide segments junctions predicted to create epitopes capable of binding human MHC class I or human MHC class II molecules.

Embodiment 249: The method of any one of embodiments 245 to 248, comprising after step b) and before step c), the steps of:
d) within the one or more regions of sequence conservation identified in step a), identifying in silico polypeptide segments predicted to bind to a human MHC class I molecule with an IC50 value of less than about 1000 nM or having a percentile rank within the top 5% in a population of polypeptide segments; and
e) generating polypeptide segments comprising the one or more regions of sequence conservation identified in step a), and which are predicted to bind to a human MHC class I molecule with an IC50 value of less than about 1000 nM or having a percentile rank within the top 5% in a population of polypeptide segments.

Embodiment 250: The method of any one of embodiments 245 to 249, further comprising after step b) and before step c) the step of reducing or eliminating viral polypeptide 9-mers that have at least 55% (5 of 9 amino acid residues), e.g., at least 65% (6 of 9 amino acid residues), e.g., at least 75% (7 of 9 amino acid residues), e.g., at least 85% (8 of 9 amino acid residues), amino acid sequence identity to a human protein.

Embodiment 251: The method of any one of embodiments 245 to 250, further comprising after step b) and before step c) the step of providing one or more polypeptide segments known or predicted to bind to a human MHC class II molecule, e.g., with a predicted binding affinity IC50 value of less than about 1000 nM or having a percentile rank within the top 5% in a population of polypeptide segments.

Embodiment 252: The method of any one of embodiments 245 to 251, further comprising after step b) and before step c) the step of identifying within the one or more regions of sequence conservation identified in step a), sequence variance in a second population of polypeptide sequences encoded by the viral gene, the second population from an intrapatient virus population.

Embodiment 253: The method of embodiment 252, wherein the sequence variance from the intrapatient virus population is determined by deep sequencing or next generation sequencing.

Embodiment 254: A method of designing a fusion polypeptide that is capable of eliciting an immune response against one or more viral target antigens, the method comprising:
a) identifying in silico one or more regions of sequence conservation in a first population of polypeptide sequences encoded by a viral gene, the first population from an interpatient virus population;
b) identifying in silico the two most prevalent polypeptide sequences from the one or more conserved regions identified in step a);
c) within the one or more regions of sequence conservation identified in step a), identifying in silico polypeptide segments predicted to bind to a human MHC class I molecule with an IC50 value of less than about 1000 nM or having a percentile rank within the top 5% in a population of polypeptide segments;
d) generating polypeptide segments comprising the one or more regions of sequence conservation identified in step a), and which are predicted to bind to a human MHC class I molecule with an IC50 value of less than about 1000 nM or having a percentile rank within the top 5% in a population of polypeptide segments;
e) removing viral polypeptide 9-mer segments generated in step d) determined to have at least 55% (5 of 9 amino acid residues), e.g., at least 65% (6 of 9 amino acid residues), e.g., at least 75% (7 of 9 amino acid residues), e.g., at least 85% (8 of 9 amino acid residues), amino acid sequence identity to a human protein, yielding retained viral polypeptide segments; and
f) arranging the retained polypeptide segments into one or more contiguous fusion polypeptides, such that the junctions connecting the polypeptide segments avoid or reduce creating epitopes capable of binding human MHC class I or human MHC class II molecules, e.g., with a predicted binding affinity IC50 value of less than about 1000 nM or having a percentile rank within the top 5% in a population of polypeptide segments.

Embodiment 255: A method of designing a fusion polypeptide that is capable of eliciting an immune response against one or more viral target antigens, the method comprising:
a) identifying in silico one or more regions of sequence conservation in a first population of polypeptide sequences encoded by a viral gene, the first population from an interpatient virus population;
b) optionally, identifying in silico the two most prevalent polypeptide sequences from the one or more conserved regions identified in step a);
c) within the one or more regions of sequence conservation identified in step a), identifying sequence variance in a second population of polypeptide sequences encoded by the viral gene, the second population from an intrapatient virus population;
d) within the one or more regions of sequence conservation identified in step a), identifying in silico polypeptide segments predicted to bind to a human MHC class I molecule with an IC50 value of less than about 1000 nM or having a percentile rank within the top 5% in a population of polypeptide segments;
e) generating polypeptide segments comprising the one or more regions of sequence conservation identified in step a), and which are predicted to bind to a human MHC class I molecule with an IC50 value of less than about 1000 nM or having a percentile rank within the top 5% in a population of polypeptide segments;
f) removing viral polypeptide 9-mer segments generated in step e) determined to have at least 55% (5 of 9 amino acid residues), e.g., at least 65% (6 of 9 amino acid residues), e.g., at least 75% (7 of 9 amino acid residues), e.g., at least 85% (8 of 9 amino acid residues), amino acid sequence identity to a human protein, yielding retained viral polypeptide segments;
g) arranging the retained polypeptide segments into one or more contiguous fusion polypeptides, such that the junctions connecting the polypeptide segments avoid or reduce creating epitopes capable of binding human MHC class I or human MHC class II molecules, e.g., with a predicted binding affinity IC50 value of less than about 1000 nM or having a percentile rank within the top 5% in a population of polypeptide segments.

Embodiment 256: The method of embodiment 255, wherein the sequence variance from the intrapatient virus population is determined by deep sequencing or next generation sequencing.

Embodiment 257: The method of any one of embodiments 254 to 256, further comprising the step of incorporating one or more polypeptide segments known or predicted to bind to a human MHC class II molecule, e.g., with a predicted binding affinity IC50 value of less than about 1000 nM or having a percentile rank within the top 5% in a population of polypeptide segments.

Embodiment 258: The method of any one of embodiments 254 to 257, further comprising the step of inserting a linker between polypeptide segments at junctions predicted to create epitopes capable of binding human MHC class I or human MHC class II molecules.

Embodiment 259: A method for producing a multivalent antigen, the method comprising constructing, in silico, a set of multivalent amino acid sequences within structurally conserved regions of a population of viral proteome sequences by a method comprising
(a) aligning the population of viral proteome sequences;
(b) creating, for each sequence in the alignment, a set of 9-amino acid subsequences (“9-mers”) starting with the N-terminal amino acid, each subsequence overlapping the preceding subsequence by eight amino acids such that each sequence of length l in the alignment contains (l-8) 9-mers;
(c) calculating a frequency for each unique 9-mer starting at a position i in each sequence of the alignment and identifying the two or more most common unique 9-mers at each position; (cxl) wherein frequency is calculated as the number of times the unique 9-mer occurs at position i in the alignment divided by the total number of sequences in the alignment;
(d) calculating a multivalent conservation for each position by summing the proportion of sequences in the alignment containing either of the two or more most common unique 9-mers;
(e) creating an alignment of conserved regions by extracting the sequences in the alignment having a multivalent conservation of greater than 80% or greater than 90%;
(f) determining a frequency for each pair of unique 9-mers at each position in the alignment of conserved regions;
(g) connecting 9-mer pairs in adjacent positions of the alignment of conserved regions that share an overlap of eight amino acids;
(h) creating a directed acyclic graph in which each 9-mer pair is a node and the edges between adjacent nodes are formed from the connected 9-mer pairs in the adjacent positions with the weight of each edge equal to the frequency of the downstream 9-mer pair,
   adding a source node and connecting it with all of the nodes in the first position,
   adding a sink node and connecting it with all of the nodes in the last position, and
   negating all of the weights;
(i) finding an optimal path in the directed acyclic graph from the source node to the sink node where the optimal path is defined in terms of the sum of the frequencies of all 9-mer pairs in the directed acyclic graph;
(j) building a multivalent antigen by connecting two or more 9-mers in adjacent positions within the optimal multivalent 9-mer path if they share an overlap of eight amino acids, thereby creating two or more sequences of connected 9-mers which together form the multivalent antigen; and
(k) optionally, rearranging the polypeptide segments to reduce or avoid the creation of deleterious epitopes at junctions between polypeptide segments.

Embodiment 260: The method of embodiment 259, wherein the multivalent conservation is bivalent conservation and wherein the multivalent antigen is a bivalent antigen.

Embodiment 261: The method of any one of embodiments 259 to 260, wherein in step (a) the conserved regions are further defined by performing one or more of the following steps:
(i) removing segments of fewer than 35 amino acids in length, e.g., from 9 amino acids to 10, 15, 20, 25, 30 or 35 amino acids in length;
(ii) removing segments determined to have less than 90% multivalent (e.g., bivalent) conservation;
(iii) removing segments determined to be weakly immunogenic or non-immunogenic, e.g., as demonstrated in in vitro or in vivo; and/or
(iv) including additional segments determined to be immunogenic, e.g., as demonstrated in in vitro or in vivo.

Embodiment 262: The method of any one of embodiments 259 to 261, wherein the step of rearranging the peptide segments to reduce or avoid creation of deleterious epitopes is performed by a method comprising one or more of in silico HLA binding analysis and human proteome cross-recognition analysis.

Embodiment 263: The method of any one of embodiments 259 to 262, further comprising inserting a linker sequence between one or more adjacent segments.

Embodiment 264: The method of any one of embodiments 259 to 263, wherein the method further comprises improving the multivalent (e.g., bivalent) antigen produced in step (h) by removing junctional 9-mers that bind to a specific HLA allele with a predicted IC50 value of less than about 1000 nM or having a percentile rank within the top 5% in a population of polypeptide segments.

Embodiment 265: The method of any one of embodiments 259 to 264, wherein the method further comprises improving the multivalent (e.g., bivalent) antigen produced in step (h) by removing 9-mers that have at least 55% (5 of 9 amino acid residues), e.g., at least 65% (6 of 9 amino acid residues), e.g., at least 75% (7 of 9 amino acid residues), e.g., at least 85% (8 of 9 amino acid residues), amino acid sequence identity with human peptides or that have the same T cell receptor (TCR) facing residues with human proteins.

Embodiment 266: The method of any one of embodiments 259 to 265, further comprising improving the multivalent (e.g., bivalent) antigen produced in step (h) to generate sufficient T cell epitopes to cover intra-patient viral diversity, the method further comprising the steps of:
a) identifying viral quasi-species variants within a biological sample obtained from a subject; and
b) determining intrapatient amino acid variants from the sequences of the multivalent (e.g., bivalent) antigen produced in step (h) by a method comprising:
   (i) determining, at each 9-mer position in the multivalent (e.g., bivalent) antigen, corresponding 9-mer subsequences from the plurality of sequencing reads that completely cover that position;
   (ii) extracting 9-mer subsequences; and
   (iii) aligning the extracted 9-mer subsequences to the sequences of the multivalent (e.g., bivalent) antigen and determining the presence of any mismatches.

Embodiment 267: The method of embodiment 266, wherein the viral quasi-species are identified by a method comprising sequencing the viral DNA, assembling a plurality of sequencing reads to create a subject consensus sequence; aligning each read in the plurality of reads to the subject consensus sequence; mapping the aligned reads of the subject to a reference sequence to obtain sequence coordinates.

Embodiment 268: The method of any one of embodiments 266 to 267, wherein the biological sample is selected from blood, peripheral blood mononuclear cells (PBMCs), serum, plasma, semen or lymph nodes.

Embodiment 269: The method of any one of embodiments 266 to 268, wherein the subject is acutely infected with HIV-1.

Embodiment 270: The method of any one of embodiments 266 to 269, wherein the subject has an HIV-1 infection of Fiebig stage IV or earlier, e.g. Fiebig stage II, Fiebig stage II or Fiebig stage I.

Embodiment 271: The method of any one of embodiments 266 to 268, wherein the subject is chronically infected with HIV-1.

Embodiment 272: The method of any one of embodiments 266 to 271, wherein the subject has received antiretroviral therapy (ART).

Embodiment 273: The method of any one of embodiments 266 to 271, wherein the subject has not received antiretroviral therapy (ART).

Embodiment 274: The method of any one of embodiments 266 to 273, further comprising excluding sequences with pre-existing escape variants.

Embodiment 275: The method of any one of embodiments 259 to 274, further comprising rearranging the polypeptide segments to reduce or avoid the creation of deleterious epitopes at junctions between polypeptide segments.

Embodiment 276: The method of embodiment 275, wherein the step of rearranging the peptide segments to reduce or avoid creation of deleterious epitopes is performed by a method comprising one or more of in silico HLA binding analysis and human proteome cross-recognition analysis.

Embodiment 277: The method of any one of embodiments 245 to 276, wherein the one or more viral target antigens are from a mammalian virus, e.g., a human virus.

Embodiment 278: The method of any one of embodiments 245 to 277, wherein the one or more viral target antigens are from a virus selected from the group consisting of human immunodeficiency virus (HIV), hepatitis B virus (HBV), human papillomavirus (HPV), herpes simplex virus (HSV), Ebola virus, Zika virus and Chikungunya virus.

Embodiment 279: The method of any one of embodiments any one of embodiments 245 to 278, wherein the interpatient virus population is from a population of patients who have not received antiretroviral therapy (ART).

Embodiment 280: The method of any one of embodiments any one of embodiments 245 to 278, wherein the interpatient virus population is from a population of patients who have received antiretroviral therapy (ART).

Embodiment 281: The method of any one of embodiments any one of embodiments 252 to 280, wherein the intrapatient virus population is from a patient who has not received antiretroviral therapy (ART).

Embodiment 282: The method of any one of embodiments any one of embodiments 252 to 280, wherein the intrapatient virus population is from a patient who has received antiretroviral therapy (ART).

Embodiment 283: A fusion polypeptide made according to the method of any one of embodiments 245 to 282, wherein the fusion polypeptide elicits an immune response against a virus in a mammal, e.g., a human.

### BRIEF DESCRIPTION OF THE DRAWINGS

FIG. 1 illustrates an 8-step workflow for designing a fusion polypeptide to elicit an antiviral response.

FIG. 2 illustrates a representative methodology of a population-based vaccine construct approach.

FIG. 3 illustrates the steps of the conserved walking analysis (CWA) algorithm, as described herein.

FIGS. 4A-4B. FIG. 4A illustrates how “bivalent conservation” can be determined based on the prevalence of the two most common 9-mers among all considered viral sequences in a population. FIG. 4A discloses SEQ ID NOS 475-476, 476-477, 476, 476, 478-479 and 479-480 in the top panel, respectively, in order of appearance. FIG. 4A also discloses “QNLQGQMVH” as SEQ ID NO: 481, “QNIQGQMVH” as SEQ ID NO: 482 and “PNIQGQMVH” as SEQ ID NO: 483 in the bottom panel. FIG. 4B illustrates how the conserved regions are identified based on the “bivalent conservation” distribution across 9-mer positions. HIV-1 Gag p24 was used as the representative protein.

FIGS. 5A-5C. FIG. 5A illustrates unique 9-mers extracted from aligned natural sequences. FIG. 5B illustrates a directed acyclic graph built based on 9-mer pair nodes and their connection. FIG. 5C illustrates how 9-mers in connected 9-mer pairs are connected. When there are two options available for the connection, the ultimate connection is determined by the prevalence of each connection in naturally occurring sequences. FIGS. 5A-5C disclose “IIIIIIIIR” as SEQ ID NO: 467, “GHHIIIIIH” as SEQ ID NO: 473, “AIHIIIIK” as SEQ ID NO: 474, “GIIIIIIIIR” as SEQ ID NO: 484, “GIIIIIIII” as SEQ ID NO: 485, “AIIIIIIII” as SEQ ID NO: 486, “IIIIIIIIK” as SEQ ID NO: 487 and “IIIIIIIIH” as SEQ ID NO: 488.

FIG. 6 illustrates interpatient and intrapatient diversity viral sequence analyses. FIG. 6 discloses SEQ ID NOs. 475-476, 476-477, 476, 476, 478-479, 479-480, and 489 in the first column and SEQ ID NOs. 475, 490, 490, 490, 490, 475, 475, 475, 475, 475, 475, 489 and 491 in the second column, all respectively, in order of appearance.

FIG. 7 illustrates intrapatient diversity analysis of HIV-1 proteins. Bivalent vaccine sequences mismatch quasi-species at 21-48% of positions in highly conserved regions.

FIG. 8 illustrates the results of human proteome cross-recognition analysis. FIG. 8 discloses the “HIV Peptide” sequence as SEQ ID NO: 492 and the “Human Protein 9-mer” sequences as SEQ ID NOS 493-498 and 493, respectively, in order of appearance.

FIG. 9 illustrates how polypeptide segment arrangement analysis can reduce or eliminate possible presentation of deleterious or undesirable epitopes injunction regions.

FIGS. 10A-10B illustrate an approach in which a set of HLA restricted 9-mers is selected from the bivalent constructs and combined to form an HLA restricted vaccine construct. FIG. 10A illustrates a basic methodology of the “short peptide” approach, described in Example 3. FIG. 10B illustrates a basic methodology of the “long peptide” approach, described in Example 3.

FIG. 11 illustrates incorporating considerations of binding of identified conserved viral protein regions to MHC class I molecules including pan-allele (e.g., HLA supertypes: A01, A02, A03, A24, B07, B08, B27, B44, B58, B62) and particular allele (e.g., A*0201 as a representative human MHC class one allele) analyses.

FIGS. 12A-12B. FIG. 12A illustrates a method of classifying conserved region positions into four categories. Antiviral vaccine design approaches can be improved by incorporating deep sequencing analysis and MHC class I binding data. FIG. 12A discloses SEQ ID NOs. 499-501, 121, 502-505, 499-501, 121 and 502-510, respectively, in order of appearance. FIG. 12B illustrates approaches of improving current antiviral vaccine design approaches by incorporating deep sequencing analysis and MHC class I binding data into intrapatient sequence analysis.

FIG. 13 illustrates an approach in which deep sequencing data and patient HLA data analyses are included to form an individualized vaccine construct.

FIG. 14 illustrates an approach in which deep sequencing data analysis is included to further improve the HLA restricted vaccine construct described in Example 3.

FIG. 15 illustrates rational antiviral immunogen design approach that considers for interpatient and intrapatient viral sequence diversity and host MHC class I and class II molecules binding, and T cell recognition. The approach and resulting immunogens are exemplified herein by immunogens that elicit human immune responses against HIV-1.

FIG. 16 illustrates polypeptide segments encoded by the HIV-1 Env gene used in the fusion polypeptide constructs described herein. The Env HIV-1 HXB2 reference polypeptide (SEQ ID NO:403) sequence is underlined. FIG. 16 also discloses SEQ ID NOs. 1, 10, 4, 15, 6, 19, 21, 27-28, 30, 37, 511, 512 and 60, respectively, in order of appearance.

FIG. 17 illustrates polypeptide segments encoded by the HIV-1 Gag gene used in the fusion polypeptide constructs described herein. The Gag HIV-1 HXB2 reference polypeptide (SEQ ID NO:404) sequence is underlined. FIG. 17 also discloses SEQ ID NOs. 70, 76, 78, 87, 94, 96-97, 99, 339, 107, 341, 117, 113, 119, 121, 123 and 137, respectively, in order of appearance.

FIG. 18 illustrates polypeptide segments encoded by the HIV-1 Nef gene used in the fusion polypeptide constructs described herein. The Nef HIV-1 HXB2 reference polypeptide, having a tryptophan (W) at position 124 (SEQ ID NO:405) sequence is underlined. FIG. 18 also discloses SEQ ID NOs. 151, 513, 153, 514, 165, 515 and 172, respectively, in order of appearance.

FIGS. 19A-19C illustrate polypeptide segments encoded by the HIV-1 Pol gene used in the fusion polypeptide constructs described herein. The Pol HIV-1 HXB2 reference polypeptide (SEQ ID NO:406) sequence is underlined. FIGS. 19A-19C also disclose SEQ ID NOs. 176, 188, 181, 190, 192, 516, 209, 517, 197, 210, 201, 211, 213, 518, 217, 219, 223, 222, 225, 227, 229-230, 232, 234, 236, 238, 240-241, 243, 259, 261, 265, 274, 282, 276, 294, 296, 300, 298, 302-303, 305, 519, 311, 319, 322-323, 334, 325, 336, 329, 327, 331 and 333, respectively, in order of appearance.

FIG. 20 illustrates modified vaccine expression cassettes for expressing the fusion polypeptides in adenoviral expression vectors, in this example, under the control of a CMV promoter. To determine an approach to combining conserved regions, candidate viral vector vaccines were constructed for expression of polypeptide segments of computationally defined conserved regions, and regions combined as (A) fusion polypeptide construct (SEQ ID NOs:345/346); (B) with a processing spacer containing the F2A proteolytic cleavage site (SEQ ID NO:349/350; (C) flexible linker (e.g., AAA (SEQ ID NO: 378)) (SEQ ID NOs:347/348); (D) fusion polypeptide with p17 and p24 conserved regions only; (E) fusion polypeptide with Protease, RT, Integrase conserved regions only; and (F) Nef only construct (SEQ ID NOs: 151/152).

FIGS. 21A-21C. (A) Plasmid DNAs containing the vaccine immunogen each representing different linkage strategies (fusion, F2A cleavage site, AAA linker (SEQ ID NO: 378)) or a fusion segment (e.g., Pol PR-RT) were transfected into Expi293™ cells. Transfection efficiency was determined by evaluation of % GFP expression of transfected Expi293F cells by flow cytometry. Results shown are representative of several independent experiments. (B) All plasmid DNAs contained p24 in the expression cassette. Expression efficiency was assessed by p24 ELISA (C) Translation products of the vaccine transgene constructs. The identity of the translation polypeptide was confirmed by Western blot immunoprecipitation with anti-Nef antibodies (2 μg/mL, 0.5 sec exposure). The uppermost band corresponded to the expected full-length translation product (88 kDa) in the fusion and the AAA linker (SEQ ID NO: 378) containing constructs. The F2A containing translation product was not detected by anti-Nef antibodies, indicating cleavage of the Nef product by absence of the full-length construct. To control for equal loading, the membrane was probed with an antibody against anti-alpha tubulin.

FIG. 22 illustrates a comparison of the differentiation phenotype of immature dendritic cells and mature monocyte derived dendritic cells (mMoDCs). Monocyte derived DCs (MoDCs) were matured in the presence of cytokines for 8 days and analyzed by flow cytometry for the expression of CD11c, HLA-DR, CD14, CD430, DCSIGN, CD83, CD86 and OX40L.

FIG. 23 illustrates representative moDC transduction efficiency using GFP expressing Ad5/35 viral vectors at multiplicity of infection (MOI) of 1000PFU in eight human donors at day 3 post-transfection. Proportion of cells expressing GFP by flow cytometry is shown on the y-axis. The x-axis represents vaccine immunogen constructs consisting of conserved regions in p17-p24 only (SEQ ID NO: 428) as well as full length Gag-Nef immunogens designed with each of the 3 fusion approaches (F2A (SEQ ID NOs: 347), fusion (SEQ ID NOs: 349) and AAA linker (SEQ ID NOs: 345) (“AAA” disclosed as SEQ ID NO: 378)). The amino acid sequences are provided in Table 1.

FIGS. 24A-24B illustrate priming of antigen specific T cells by vaccine constructs expressing conserved regions of HIV-1 concatenated or connected by fusion, F2A proteolytic cleavage sequence or an AAA linker (SEQ ID NO: 378). (A) Magnitude of primed responses assessed by IFN-γ ELISpot assay on day 10 following co-culture of PBMCs with vaccine vector transduced autologous moDCs. PBMCs derived from HIV infected donors with pre-existing HIV specific responses at baseline (B) Breadth of responses defined as number of de novo recognized peptide pools (excluding pre-existing baseline responses). No statistical significance was observed among the different groups in this analysis.

FIGS. 25A-25B illustrate in vitro priming of antigen specific T cells from HIV infected donors. Representative flow cytometry plots showing IFN-γ production by intracellular cytokine staining (ICS) on day 10 following co-culture of PBMCs with vaccine vector transduced autologous moDCs. The x-axis indicates the vaccine construct used for in-vitro priming. Each bar represents stimulation with peptide pools from Gag p17, gag p24, integrase, Pol (protease/RT) and Nef respectively. Percentages of IFN-γ+ T cells are indicated (A) CD8+ T cell responses (B) CD4+ T cells. (●) Gag p17; (▪) Gag p24; (▴) NT; (▾) Pol; (♦) Nef. No statistical significance was observed among the different groups in this analysis. FIGS. 25A-25B disclose “AAA” as SEQ ID NO: 378.

FIGS. 26A-26B illustrate in vitro priming of antigen specific T cells from aviremic and viremic HIV-1 infected donors. Representative figures showing IFN-γ production by intracellular cytokine staining (ICS) on day 7 (first round) and day 14 (second round) following co-culture of PBMCs with autologous moDCs transduced with an adenoviral vector comprising a Gag-Nef immunogen designed according to one of three fusion approaches (F2A (SEQ ID NO: 347), fusion (SEQ ID NO: 349) and AAA linker (SEQ ID NO: 345; “AAA” disclosed as SEQ ID NO: 378) in aviremic (A) and viremic (B) donors. The x-axis indicates the vaccine construct used for in-vitro priming. Each bar represents stimulation with peptide pools from Gag p17, Gag p24, integrase, Pol (protease/RT) and Nef respectively. Percentages of IFN-7+ T cells are indicated (A) CD8+ T cell responses (B) CD4+ T cells. (●) Gag p17; (▪) Gag p24; (▴) INT; (▾) Pol; (♦) Nef.

FIG. 27 illustrates profiling of functional characteristics of antigen of antigen specific CD8+ and CD4+ T cells primed using moDCs transduced with vectors containing immunogen expression cassettes with conserved regions concatenated by fusion, F2A proteolytic cleavage sequence or AAA linker (SEQ ID NO: 378). The pie charts depict adjusted multifunctionality (background responses subtracted) characteristics of primed CD8+ or CD4+ T cells (one to four functions—CD107a, IFN-γ, TNF-α and IL-2).

FIGS. 28A-28C illustrate viral vectors expressing HIV-1 antigens elicit high magnitude CD8+ T cell responses following immunization. (A) Immunization and sampling schedule. Groups of Balb/c mice were immunized with Ad5/35 vectors expressing HIV-1 conserved regions sequences concatenated by fusion, F2A proteolytic cleavage sequence or a flexible AAA linker (SEQ ID NO: 378). Mice were immunized in a homologous prime-boost schedule on day 1 and day 29, with analysis of each group on Day 16 (prime) or Day 36 (prime-boost). (B) Day 16 (C) Day 36 immunogenicity following immunization was determined by evaluating the frequency of ex vivo peptide specific splenocytes using an IFN-γ ELISPOT assay to detect IFN-γ producing cells. A set of 15-mer peptides overlapping by 11 amino acids that matched the sequences within the vaccine construct as well as the F2A peptide were synthesized and used to stimulate splenocytes in the ELISpot and ICS assays.

FIGS. 29A-29B illustrate functional profiles of vaccine induced CD8+ T cell responses in Balb/c (A) and C57 BL/6 animals (B). Flow cytometry profiles of CD8+ T cells able to mediate degranulation (CD107a), IFN-γ, IL-2 and TNF-α production were analyzed and the functional composition of responses for animals in each vaccination group are shown. Splenocytes were stimulated with the relevant peptide pool (p24 shown here) for 6 hours and stained as described in methods. The pie chart summarized the data, with each slice of the pie corresponding to the fraction of CD8+ T cells with a given number of functions within the total CD8+ T cell population. All possible combinations of responses are shown on the x-axis, and the percentage of functionally distinct CD8+ T cells within the total population are shown on the y-axis. Mean and SD are shown. FIGS. 29A-29B disclose “AAA” as SEQ ID NO: 378.

FIGS. 30A-30B illustrate the memory phenotype of IFN-γ producing cells. (A) Flow cytometry plots illustrating the gating strategy to define memory subsets and exhaustion phenotype based on CCR7, CD45RA, CD27, PD-1 and CTLA-4 expression on IFN-γ+CD8+ T cells post re-stimulation with 2 μg/ml Gag p24 peptide pools. (B) The proportion of naive (CCR7+CD45RA+), effector memory (CCR7− CD45RA−) and central memory cells (CCR7+CD45RA−) within IFN-γ+CD8+ and IFN-γ+CD4+ T cells post re-stimulation with 2 μg/ml Gag p24 peptide pools.

FIGS. 31A-31B. FIG. 31A illustrates that signal sequences differentially enhance immunogenicity of vaccine immunogen (SEQ ID NOs: 369, 370, 371, 368, 367). FIG. 31B illustrates that sequences of HIV-1 conserved regions are immunogenic with GM-CSF signal sequence (SEQ ID NOs: 353, 363).

FIGS. 32A-32E. FIG. 32A illustrates immunization and sampling schedule. FIG. 32B illustrates LCMV vectors expressing HIV-1 conserved regions sequences from Gag-Nef fusion protein sequences without a signal sequence (SEQ ID NOs: 357, 430) and with signal sequences from GM-CSF (SEQ ID NOs: 353, 363), t-PA (SEQ ID NO: 354), MCP-3 (SEQ ID NO: 355), β-catenin (SEQ ID NO: 356) used to immunize groups of Balb/c mice. FIGS. 32C-32D illustrate the immunogenicity of vaccine immunogens with different signal sequences to Gag p24 by IFN-γ ELISpot after prime immunization on day 7 (FIG. 32C) and boost on day 27 (FIG. 32D) with LCMV replication incompetent vectors expressing conserved Gag-Nef in the presence or absence of MCP-3, tPA, β-catenin and GM-CSF signal sequences. FIG. 32E represents immunogenicity of Ad5/35 vectors expressing conserved Pol with or without GMCSF signal sequence in female Balb/c on day 7 post immunization. Each point represents one individual mouse. Mean and SD are shown. No statistical significance was observed among the different groups in this analysis.

FIGS. 33A-33D. FIG. 32A illustrates immunization and sampling schedule. Groups of Balb/c mice were immunized with FIG. 33B illustrates Ad5/35 vectors expressing HIV-1 conserved regions from Gag-Nef and Pol fusion protein sequences without a signal sequence (SEQ ID NOs: 357, 430) and with signal sequences from GM-CSF (SEQ ID NOs: 353, 363), t-PA (SEQ ID NO: 354), MCP-3 (SEQ ID NO: 355), β-catenin (SEQ ID NO: 356) used to immunize groups of Balb/c mice. Fusion protein sequences are provided in Table J. FIGS. 33C-33D illustrate the immunogenicity of vaccine immunogens with different signal sequences by IFN-γ ELISpot (FIG. 33C) and intracellular IFN-γ+CD8+T (FIG. 33D) cells by flow cytometry analysis after prime immunization on day 16. Each point represents one individual mouse. Mean and SD are shown. Non-parametric Mann-Whitney tests were used to determine statistical significance between groups. *P≤0.05, **P≤0.001.

FIGS. 34A-34D illustrate immunogenicity of LCMV vectors containing conserved HIV sequences with and without leader sequences in A*0201-C57/BL6 transgenic mice. FIG. 34A represents the immunization and sampling schedules depicting time points for prime and boost vaccination and assessment of responses by IFN-γ ELISpot. FIG. 34B represents immunogen constructs used for vaccinations, with LCMV vectors expressing A*0201 sequences, in the absence (SEQ ID NOs: 367, 431) or presence (SEQ ID NOs: 368, 432) of GM-CSF signal sequence. Mice were also immunized with LCMV vectors expressing Gag-Nef and Pol fusion protein sequences in the absence (SEQ ID NO: 430+SEQ ID NO: 357) or presence (SEQ ID NO: 353+SEQ ID NO: 363) of GM-CSF signal sequences. Fusion protein sequences are provided in Table J. The A*0201 sequences comprise specific epitope sequences from conserved HIV sequences against A*0201 allele and are placed in the vector as bead in a string arrangement. FIG. 34C represents magnitude of IFN-γ responses against A*0201 peptide pool from both prime and prime/boost vaccinated animals. FIG. 34D represents magnitude of IFN-γ responses against Gag peptide pool from both prime and prime/boost vaccinated animals. In FIGS. 34C-34D: the Y axis represents magnitude of IFN-γ responses against the specific peptide pool stimulus as number of spot-forming units (SFU) per 10⁶ Splenocytes. Peptide specific values were obtained by subtracting no peptide stimulated control to exclude nonspecific responses. The X-axis indicates the individual vaccine constructs used for in vivo priming and boosting against which peptide specific responses were studied. No statistical significance was observed among the different groups in this analysis.

FIGS. 35A-35D illustrate immunogenicity of Ad5/35 vectors containing conserved HIV sequences with and without leader sequences in A*0201-C57/BL6 transgenic mice. FIG. 35A represents the immunization and sampling schedules depicting time points for prime vaccination and assessment of responses by IFN-γ ELISpot. FIG. 35B represents immunogen constructs used for vaccinations, where A*0201 sequences without a signal sequence is (SEQ ID NOs: 367, 431), with GM-CSF signal sequence is (SEQ ID NOs: 368, 432), with tPA signal sequence is (SEQ ID NOs: 369, 433), with MCP-3 signal sequence is (SEQ ID NOs: 370, 434), with β-catenin signal sequence is (SEQ ID NOs: 371, 435), with LAMP-1 N-terminal and C-terminal signal sequences is (SEQ ID NO: 372) and vectors each expressing GM-CSF Gag-Nef and GM-CSF Pol fusion protein sequences (SEQ ID NO: 353+SEQ ID NO: 363) respectively. Fusion protein sequences are provided in Table J. The A*0201 sequences in vectors comprise specific epitope sequences from conserved HIV sequences against A*0201 allele and are cloned in the Ad5/35 vectors as a bead on a string arrangement. FIG. 35C represents magnitude of IFN-γ responses against A*0201 peptide pool from prime vaccinated animals. The Y axis represents magnitude of IFN-γ responses against the A*0201 peptide pool stimulus as number of spot-forming cells (SFC) per 10⁶ splenocytes. Peptide specific values were obtained by subtracting no peptide stimulated control to exclude nonspecific responses. The X-axis indicates the individual vaccine constructs used for in-vivo priming. FIG. 35D represents magnitude of IFN-γ responses against Gag, Nef, Pol-1 and Pol-2 peptide pools in GM-CSF-Gag/Nef+GM-CSF-Pol vaccinated animals. The vector sequences contain conserved sequences as a whole; not just the A*0201 epitope specific sequences. The Y axis represents magnitude of IFN-γ responses in GM-CSF-Gag/Nef+GM-CSCF-Pol vaccine primed animals and X axis represents specific peptide pools used in stimulation. Each bar represents stimulation with peptide pools from Gag p24, Gag p17, Nef, Pol-1(protease/RT) and Pol-2 (integrase) responses. Responses are represented as spot forming units (SFU) per 10⁶ Splenocytes. Peptide specific values were obtained by subtracting no peptide stimulated control to exclude nonspecific responses. Non-parametric Mann-Whitney tests were used to determine statistical significance between groups. *P≤0.05, **P≤0.001.

FIGS. 36A-36E illustrate various prime and prime-boost regimens using arenavirus LCMV and Pichinde (PICV) arenavirus vectors. (A) C57Bl/6 mice were immunized with a single prime with tri-segmented replication attenuated LCMV (TT1), tri-segmented replication attenuated PICV (TT2), replication defective PICV (VV2) and adenovirus vectors expressing SIVsme543 gp140. (B) Homologous and heterologous prime boost regimens with tri-segmented replication attenuated LCMV (TT1) or tri-segmented replication attenuated PICV (TT2) vectors expressing Gag, Pol-1/Pol-2 and Env (gp140). (C) Comparison of heterologous prime-boost, first priming with tri-segmented replication attenuated PICV (TT2) or replication defective PICV (VV2) and then boosting with replication defective LCMV (VV1) and tri-segmented replication attenuated LCMV (TT1) vectors expressing SIV antigens. Mice received a single immunization at each time point consisting of three (tri-segmented replication attenuated) or four (replication deficient) arenavirus vectors mixed in a 1:1:1:1 ratio. (D) Immunization of rhesus macaques with replication attenuated arenavirus vectors expressing SIVsme543 Gag as homologous LCMV or PICV and heterologous PICV prime and LCMV boost. Four intravenous immunizations we administered on Days 1, 29, 85 and 113. (E) Summary of longitudinal follow-up immune responses by IFN-γ ELISpot in rhesus macaques immunized with replication attenuated arenavirus vectors expressing SIVsme543 Gag as homologous LCMV or PICV and heterologous PICV prime and LCMV boost. The responses observed in (E) are an extension of the responses observed in (D).

FIGS. 37A-37F. (A) Illustrates protocol established for moDC-T cell priming assay followed by individual epitope using 384 well ELISPOT assays. (B) Represents the gender, viral loads and HLA diversity characteristics currently available for 10 aviremic HIV-1 patient donors completed in this analysis. (C) Represents viral vector sequences in the absence of signal sequences to evaluate if vaccination enhances the breadth of response evaluated in (D). (D) Comparison of the breadth of responses (number of independent epitopes) induced post priming with moDCs transduced with Ad5/35 vectors expressing conserved regions within Gag-Nef and Pol-Env. (E) Characterization of the breadth of immune responses targeted to different HIV-1 antigens with empty vectors (pre-vaccine) and conserved regions vaccines with different signal sequences (SEQ ID NOs. 353, 363, 354, 355, 356, 429 and 357). (F) Breadth of responses defined as number of de novo recognized peptide pools (excluding pre-existing baseline responses) and magnitude of responses assessed by IFN-γ ELISpot assay on day 10 following co-culture of PBMCs with vaccine vector transduced autologous moDCs expressing conserved regions constructs with different signal sequences. Each point represents one donor. Mean and SD are shown.

### DETAILED DESCRIPTION

### 1. Introduction

Provided herein are fusion polypeptides comprising a plurality of polypeptide or peptide segments and related compositions, including immunogenic compositions and pharmaceutical compositions, as well as methods for making the fusion polypeptides and methods for their use to elicit an immunogenic response to a human immunodeficiency virus (HIV-1) in a subject in need thereof. As used herein, an “immunogen” is a substance, such as an antigen, that elicits an immune response or is capable of eliciting an immune response. Also provided are polynucleotides encoding the fusion polypeptides described herein, as well as vectors comprising same.

Provided herein are fusion polypeptides designed to induce an antiviral immune response. The vaccine constructs described herein were designed to provide mathematically-determined improved coverage of predicted T cell epitopes (“PTE”) using the most highly conserved predicted epitopes within a source set of viral proteome sequences. As a paradigm for the methods of designing antiviral immunogens, fusion polypeptides encoded by one, two, three, or four, of the HIV-1 Gag, Pol, Env, and Nef genes were used. The methods described herein both retain the positional information of the PTE's within the source set of sequences and construct a bivalent set of sequences to improve coverage of conserved PTEs. The result is an initial bivalent vaccine construct that advantageously improves or increases highly conserved PTEs that are most likely to be highly similar to conserved epitopes in the naturally occurring sequences in proteins expressed by viral species amongst a population of patients and within an individual patient, due to both the retained positional information. In addition, the use of only highly conserved PTE sequences amongst HIV-1 species in interpatient and intrapatient populations reduces the likelihood of escape mutants because the highly conserved sequences are more likely to contribute viral structure and function.

Further provided are computational approaches for designing antiviral vaccine immunogens for a highly variable virus, such as HIV-1. The antiviral vaccine immunogen design methods incorporate deep sequencing data from individual patient samples with variable sequences and analyze the sequence diversity in the context of host HLA diversity to develop antiviral vaccines for therapeutic and preventative use. The antiviral immunogens can be designed to provide coverage at an individual level, for a group of individuals with a defined set of HLA alleles, or for broad population coverage. In the herein described vaccine immunogen design methods, we define a computational approach for targeting conserved regions within a vaccine sequence using bulk population sequences, e.g., from public databases and internally developed databases. Further, using individual patient deep sequence data we define sequence variability for each potential T cell epitope within the conserved regions. Moreover, we identify regions that may serve as actual epitopes based on likelihood of presentation by the individual host's set of HLA alleles. The likelihood of binding to host HLA defined by publicly available and internally-developed databases, was used to develop deep learning models that model peptide binding per allele. This can be coupled with in-silico, published and/or experimental in-vitro T cell priming data that can define the potential impact of antigen variants in modulating TCR recognition or identify a peptide as an escape variant. These data are used to design a set of peptide immunogens that contain the epitopes and associated epitope variants. The epitope sequences are concatenated or connected in series into a single fusion polypeptide, either directly fused or linked via a linker sequence. Peptide segments are joined in a computationally determined sequential order from N-terminus to C-terminus that reduces or eliminates the creation of junctional epitopes that may mimic human self-antigens and have undesirable effects (e.g., eliciting an autoimmune response or a tolerogenic response).

Unlike similar graph-based approaches to vaccine design, the approaches described herein build segments of connected PTE's using only adjacent PTE's that are also adjacent in the natural sequences. In addition, the present methods first build a bivalent construct consisting of two polypeptides matched to improve or increase coverage at each PTE position in the viral proteome. The bivalent construct itself may be used as a vaccine, as in the constructs described in Examples 1 and 2 below, or it may serve as the basis for a further construct, such as the HLA-restricted construct described in Example 3 or individualized construct described in Examples 4 and 5, below. The bivalent constructs designed by analysis of population-based sequences (e.g., interpatient diversity) identifies population-based conserved sequences that may contribute to viral structure and function and can serve as a template for defining conservation within intrapatient sequences, which information can be applied to build an individualized vaccine construct.

The methods described herein can begin with the identification of conserved region bivalent sequences, using a process referred to herein as the “Conservation Analysis” or “Conservation Algorithm.” The methods further can comprise a step of building a bivalent vaccine construct having maximal epitope coverage while retaining the positional information of the PTE's from the natural sequences, using a process referred to referred to herein as a “Conserved Walking Algorithm” or “CWA.”

Therapeutic vaccines based on the induction of T cell responses are being readily evaluated and showing efficacy within the context of cancer vaccines. These vaccines are typically epitope based and can be tailored to an individual's HLA alleles as well as their specific tumor antigen (see, e.g., Tran, et al., Science, (2014) 344(6184):641-5). We have developed vaccine sequences that may target sub-populations based on the population prevalence of certain HLA alleles or may be specifically tailored to an individual's range of HLA alleles.

### 2. Fusion Polypeptides Useful to Promote Immune Response Against Human Immunodeficiency Virus-1 (HIV-1)

Provided herein are fusion polypeptides comprising a plurality of polypeptide or peptide segments encoded by one or more HIV-1 genes. A ‘segment’ of a fusion polypeptide described herein is a contiguous sequence of at least 8 amino acids with respect to a reference sequence, for example HIV-1 HXB2 reference sequences for Env, Gag, Nef and Pol polypeptides, provided herein as SEQ ID NOs: 403-406, respectively. The polypeptides described herein are ‘fusion’ polypeptides in the sense that they are assembled from connected or concatenated polypeptide or peptide segments of two or more HIV-1 proteins. With respect to the HIV-1 protein reference sequences, the polypeptide or peptide segments may correspond to discontinuous sequences of the same HIV-1 protein or different HIV-1 proteins. Generally, the fusion polypeptides are non-naturally occurring, and can be synthetic or recombinantly produced.

a. Polypeptide Segments

With respect to the HIV-1 genes encoding the polypeptide segments used to assemble the herein described fusion polypeptides, in various embodiments, the fusion polypeptides comprise a plurality of polypeptide segments of one or more human immunodeficiency virus-1 (HIV-1) proteins encoded by one or more, e.g. two or more, three or more, four or more, HIV-1 genes selected from Gag, Nef, Env, Pol, Vpu, Vpr and Vif, e.g., two or more, three or more, four or more, HIV-1 genes selected from Gag, Nef, Env, Pol, and Vif. In some embodiments, the plurality of polypeptide segments is comprised of only polypeptide segments encoded by HIV-1 genes Env, Gag, Nef and Pol, e.g., does not comprise polypeptide segments encoded by HIV-1 Tat, Rev, Vif, Vpr and/or Vpu genes. In some embodiments, the plurality of polypeptide segments is comprised of only polypeptide segments encoded by HIV-1 genes Gag, Nef and Pol, e.g., does not comprise polypeptide segments encoded by HIV-1 Env, Tat, Rev, Vif, Vpr and/or Vpu genes. In some embodiments, the plurality of polypeptide segments is comprised of only polypeptide segments encoded by HIV-1 genes Gag and Nef, e.g., does not comprise polypeptide segments encoded by HIV-1 Env, Pol, Tat, Rev, Vif, Vpr and/or Vpu genes. In some embodiments, the plurality of polypeptide segments is comprised of only polypeptide segments encoded by HIV-1 genes Pol and Nef, e.g., does not comprise polypeptide segments encoded by HIV-1 Env, Gag, Tat, Rev, Vif, Vpr and/or Vpu genes. In some embodiments, the plurality of polypeptide segments is comprised of only polypeptide segments encoded by HIV-1 genes Pol and Env, e.g., does not comprise polypeptide segments encoded by HIV-1 Gag, Nef, Tat, Rev, Vif, Vpr and/or Vpu genes. In some embodiments, the plurality of polypeptide segments is comprised of only polypeptide segments encoded by HIV-1 Pol gene, e.g., does not comprise polypeptide segments encoded by HIV-1 Env, Gag, Nef, Tat, Rev, Vif, Vpr and/or Vpu genes. In various embodiments, the plurality of polypeptide segments does not contain a segment encoded by one, two, three or four of HIV Tat, Rev, Vif, Vpr and/or Vpu genes.

With respect to the number of polypeptide segments assembled, connected, linked or concatenated into a single fusion polypeptide, in various embodiments, the fusion polypeptides are comprised of at least 5 and up to 40 polypeptide segments, e.g., from 5 polypeptide segments and up to 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39 or 40 polypeptide segments. As appropriate, the polypeptide segments can be arranged in the same order or according to a different order than in the naturally occurring proteins.

With respect to the regions of the polypeptides encoded by an HIV-1 gene selected as polypeptide segments to include in the fusion polypeptides, in various embodiments, the polypeptide segments are derived from conserved regions in a population of viral proteome sequences. In some embodiments, the conserved regions are greater than 80%, e.g., greater than 81%, 82%, 83%, 84%, 85%, 86%, 87%, 88%, 89%, 90%, 91%, 92%, 93%, 94%, 95%, 96%, 97%, 98% or 99% conserved amongst HIV-1 species, e.g., as determined in interpatient and/or intrapatient populations. As used herein, conserved regions in a polypeptide encoded by an HIV-1 gene refers to the percentage of sequences in a population of sequences containing identical amino acid segments or subsequences e.g., (segments 9 amino acids in length or 9-mers) as the most prevalent one in a predetermined amino acid segment or subsequence position, where an amino acid segment or subsequence position is determined with respect to a reference sequence, e.g., HIV-1 HXB2 polypeptide sequences, e.g., SEQ ID NOs: 403-406. In various embodiments, the conserved regions are conserved amongst one or more of HIV-1 clades within Group M, e.g., one or more of HIV-1 clades A-K, e.g., one or more of clades A, B, C, D and G, e.g., amongst HIV-1 Group M, clade B, and recombinant forms thereof, e.g., CRF01_AE. In some embodiments, the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 1-344, e.g., polypeptide segments identified in Table B. In some embodiments, the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, polypeptide segments selected from SEQ ID NOs: 2, 3, 8, 9, 13, 14, 17, 18, 23, 24, 25, 26, 28, 29, 31, 32, 33, 34, 35, 36, 37, 38, 39, 40, 43, 44, 45, 46, 47, 48, 49, 50, 51, 52, 53, 54, 55, 56, 58, 59, 62, 63, 64, 65, 66, 67, 68, 69, 72, 73, 74, 75, 76, 77, 80, 81, 82, 83, 84, 85, 86, 87, 88, 89, 92, 93, 101, 102, 103, 104, 109, 110, 115, 116, 125, 126, 127, 128, 129, 130, 131, 132, 133, 134, 135, 136, 139, 140, 141, 142, 143, 144, 145, 146, 147, 148, 149, 150, 155, 156, 157, 158, 159, 160, 166, 167, 168, 169, 170, 171, 174, 175, 178, 179, 180, 181, 182, 183, 184, 185, 193, 194, 195, 196, 197, 198, 199, 200, 203, 204, 205, 206, 207, 208, 213, 214, 221, 222, 236, 237, 245, 246, 247, 248, 249, 250, 251, 252, 253, 254, 255, 256, 257, 258, 263, 264, 266, 267, 268, 269, 270, 271, 272, 273, 278, 279, 280, 281, 282, 283, 284, 285, 286, 287, 288, 289, 290, 291, 292, 293, 294, 295, 305, 306, 307, 308, 309, 310, 313, 314, 315, 316, 317, 318, 321 and 322, e.g., polypeptide segments identified in Table C. The start and end positions are with respect to HIV-1 HXB2 reference polypeptides, GenBank Accession No. K03455 (ncbi.nlm.nih.gov/nuccore/K03455), provided herein as SEQ ID NOs: 403-406 and identified in Table A.

TABLE A
HIV-1 HXB2 reference sequences

| SEQ ID | | |
|---|---|---|
| NO: | GENE | SEQUENCE |
| 403 | Env | MRVKEKYQHLWRWGWRWGTMLLGMLMICSATEKLWVTVYYGVPVWKEATTTLFCASDAKAYDT |
| | | EVHNVWATHACVPTDPNPQEVVLVNVTENFNMWKNDMVEQMHEDIISLWDQSLKPCVKLTPLC |
| | | VSLKCTDLKNDTNTNSSSGRMIMEKGEIKNCSFNISTSIRGKVQKEYAFFYKLDIIPIDNDTT |
| | | SYKLTSCNTSVITQACPKVSFEPIPIHYCAPAGFAILKCNNKTFNGTGPCTNVSTVQCTHGIR |
| | | PVVSTQLLLNGSLAEEEVVIRSVNFTDNAKTIIVQLNTSVEINCTRPNNNTRKRIRIQRGPGR |
| | | AFVTIGKIGNMRQAHCNISRAKWNNTLKQIASKLREQFGNNKTIIFKQSSGGDPEIVTHSFNC |
| | | GGEFFYCNSTQLFNSTWENSTWSTEGSNNTEGSDTITLPCRIKQIINMWQKVGKAMYAPPISG |
| | | QIRCSSNITGLLLTRDGGNSNNESEIFRPGGGDMRDNWRSELYKYKVVKIEPLGVAPTKAKRR |
| | | VVQREKRAVGIGALFLGFLGAAGSTMGAASMTLTVQARQLLSGIVQQQNNLLRAIEAQQHLLQ |
| | | LTVWGIKQLQARILAVERYLKDQQLLGIWGCSGKLICTTAVPWNASWSNKSLEQIWNHTTWME |
| | | WDREINNYTSLIHSLIEESQNQQEKNEQELLELDKWASLWNWFNITNWLWYIKLFIMIVGGLV |
| | | GLRIVFAVLSIVNRVRQGYSPLSFQTHLPTPRGPDRPEGIEEEGGERDRDRSIRLVNGSLALI |
| | | WDDLRSLCLFSYHRLRDLLLIVTRIVELLGRRGWEALKYWWNLLQYWSQELKNSAVSLLNATA |
| | | IAVAEGTDRVIEVVQGACRAIRHIPRRIRQGLERILL |
| 404 | Gag | MGARASVLSGGELDRWEKIRLRPGGKKKYKLKHIVWASRELERFAVNPGLLETSEGCRQILGQ |
| | | LQPSLQTGSEELRSLYNTVATLYCVHQRIEIKDTKEALDKIEEEQNKSKKKAQQAAADTGHSN |
| | | QVSQNYPIVQNIQGQMVHQAISPRTLNAWVKVVEEKAFSPEVIPMFSALSEGATPQDLNTMLN |
| | | TVGGHQAAMQMLKETINEEAAEWDRVHPVHAGPIAPGQMREPRGSDIAGTTSTLQEQIGWMTN |
| | | NPPIPVGEIYKRWIILGLNKIVRMYSPTSILDIRQGPKEPFRDYVDRFYKTLRAEQASQEVKN |
| | | WMTETLLVQNANPDCKTILKALGPAATLEEMMTACQGVGGPGHKARVLAEAMSQVTNSATIMM |
| | | QRGNFRNQRKIVKCFNCGKEGHTARNCRAPRKKGCWKCGKEGHQMKDCTERQANFLGKIWPSY |
| | | KGRPGNFLQSRPEPTAPPEESFRSGVETTTPPQKQEPIDKELYPLTSLRSLFGNDPSSQ |
| 405 | Nef | MGGKWSKSSVIGWPTVRERMRRAEPAADRVGAASRDLEKHGAITSSNTAATNAACAWLEAQEE |
| | | EEVGFPVTPQVPLRPMTYKAAVDLSHFLKEKGGLEGLIHSQRRQDILDLWIYHTQGYFPDWQN |
| | | YTPGPGVRYPLTFGWCYKLVPVEPDKIEEANKGENTSLLHPVSLHGMDDPEREVLEWRFDSRL |
| | | AFHHVARELHPEYFKNC |
| 406 | Pol | FFREDLAFLQGKAREFSSEQTRANSPTRRELQVWGRDNNSPSEAGADRQGTVSFNFPQVTLWQ |
| | | RPLVTIKIGGQLKEALLDTGADDTVLEEMSLPGRWKPKMIGGIGGFIKVRQYDQILIEICGHK |
| | | AIGTVLVGPTPVNIIGRNLLTQIGCTLNFPISPIETVPVKLKPGMDGPKVKQWPLTEEKIKAL |
| | | VEICTEMEKEGKISKIGPENPYNTPVFAIKKKDSTKWRKLVDFRELNKRTQDFWEVQLGIPHP |
| | | AGLKKKKSVTVLDVGDAYFSVPLDEDFRKYTAFTIPSINNETPGIRYQYNVLPQGWKGSPAIF |
| | | QSSMTKILEPFRKQNPDIVIYQYMDDLYVGSDLEIGQHRTKIEELRQHLLRWGLTTPDKKHQK |
| | | EPPFLWMGYELHPDKWTVQPIVLPEKDSWTVNDIQKLVGKLNWASQIYPGIKVRQLCKLLRGT |
| | | KALTEVIPLTEEAELELAENREILKEPVHGVYYDPSKDLIAEIQKQGQGQWTYQIYQEPFKNL |
| | | KTGKYARMRGAHTNDVKQLTEAVQKITTESIVIWGKTPKFKLPIQKETWETWWTEYWQATWIP |
| | | EWEEVNTPPLVKLWYQLEKEPIVGAETFYVDGAANRETKLGKAGYVTNRGRQKVVTLTDTTNQ |
| | | KTELQAIYLALQDSGLEVNIVTDSQYALGIIQAQPDQSESELVNQIIEQLIKKEKVYLAWVPA |
| | | HKGIGGNEQVDKLVSAGIRKVLFLDGIDKAQDEHEKYHSNWRAMASDFNLPPVVAKEIVASCD |
| | | KCQLKGEAMHGQVDCSPGIWQLDCTHLEGKVILVAVHVASGYIEAEVIPAETGQETAYFLLKL |
| | | AGRWPVKTIHTDNGSNFTGATVRAACWWAGIKQEFGIPYNPQSQGVVESMNKELKKIIGQVRD |
| | | QAEHLKTAVQMAVFIHNFKRKGGIGGYSAGERIVDIIATDIQTKELQKQITKIQNFRVYYRDS |
| | | RNPLWKGPAKLLWKGEGAVVIQDNSDIKVVPRRKAKIIRDYGKQMAGDDCVASRQDED |

TABLE B
all polypeptide segments

| SEQ ID | | | | |
|---|---|---|---|---|
| NO | Gene | Start | End | Sequence |
| 1 | Env | 28 | 52 | CSATEKLWVTVYYGVPVWKEATTTL |
| 2 | Env | 34 | 48 | LWVTVYYGVPVWKEA |
| 3 | Env | 34 | 48 | LWVTIYYGVPVWKDA |
| 4 | Env | 34 | 47 | LWVTVYYGVPVWKE |
| 5 | Env | 34 | 47 | LWVTIYYGVPVWKD |
| 6 | Env | 36 | 44 | VTVYYGVPV |
| 7 | Env | 36 | 44 | VTIYYGVPV |
| 8 | Env | 48 | 61 | ATTTLFCASDAKAY |
| 9 | Env | 48 | 61 | ANTTLFCASDAKGY |
| 10 | Env | 59 | 83 | KAYDTEVHNVWATHACVPTDPNPQE |
| 11 | Env | 64 | 83 | AHNVWATHACVPTDPNPQE |
| 12 | Env | 64 | 83 | VHNIWATHACVPTDPSPQE |
| 13 | Env | 65 | 83 | HNVWATHACVPTDPNPQE |
| 14 | Env | 65 | 83 | HNIWATHACVPTDPSPQE |
| 15 | Env | 67 | 75 | NVWATHACV |
| 16 | Env | 67 | 75 | NIWATHACV |
| 17 | Env | 107 | 129 | DIISLWDQSLKPCVKLTPLCVTL |
| 18 | Env | 107 | 129 | DIISLWDESLKPCVKLTPICVTL |
| 19 | Env | 113 | 137 | DQSLKPCVKLTPLCVTLNCTDLRNT |
| 20 | Env | 113 | 137 | DESLKPCVKLTPICVTLNCTDLRNT |
| 21 | Env | 121 | 129 | KLTPLCVTL |
| 22 | Env | 121 | 129 | KLTPICVTL |
| 23 | Env | 209 | 226 | SFEPIPIHYCAPAGFAIL |
| 24 | Env | 209 | 226 | TFEPIPIHYCTPAGFAIL |
| 25 | Env | 220 | 228 | PAGFAILKC |
| 26 | Env | 220 | 228 | PAGFALLKC |
| 27 | Env | 235 | 259 | GTGPCTNVSTVQCTHGIRPVVSTQL |
| 28 | Env | 241 | 268 | NVSTVQCTHGIRPVVSTQLLLNGSLAEE |
| 29 | Env | 241 | 268 | NISTVQCTHGIKPVVSTQLLLNGSLAEK |
| 30 | Env | 243 | 251 | STVQCTHGI |
| 31 | Env | 376 | 386 | FNCGGEFFYCN |
| 32 | Env | 376 | 386 | FNCRGEFFYCN |
| 33 | Env | 430 | 439 | VGKAMYAPPI |
| 34 | Env | 430 | 439 | VGRAMYAPPI |
| 35 | Env | 472 | 481 | GGDMRDNWRS |
| 36 | Env | 472 | 481 | GGNMKDNWRS |
| 37 | Env | 475 | 489 | MRDNWRSELYKYKVV |
| 38 | Env | 475 | 489 | MKDNWRSELYRYKVV |
| 39 | Env | 501 | 511 | AKRRVVQREKR |
| 40 | Env | 501 | 511 | ARRRVVQREKR |
| 41 | Env | 502 | 606 | KRRVVQREKRAVGIGAMFLGFLGAAGSTMGAASITLTVQARQLLSGIVQQQNN |
| | | | | LLRAIEAQQHLLQLTVWGIKQLQARVLAVERYLKDQQLLGIWGCSGKLICTT |
| 42 | Env | 502 | 606 | RRRVVQREKRAIGLGAVFLGFLGTAGSTMGAASMTLTVQARLLLSGIVQQQSN |
| | | | | LLRAIEAQQHMLQLTVWGIKQLQARILAVERYLRDQQLLGIWGCSGRLICTT |
| 43 | Env | 519 | 534 | FLGFLGAAGSTMGAAS |
| 44 | Env | 519 | 534 | FLGFLGTAGSTMGAAA |
| 45 | Env | 533 | 606 | ASITLIVQARQLLSGIVQQQNNLLRAIEAQQHLLQLTVWGIKQLQARVLAVER |
| | | | | YLKDQQLLGIWGCSGKLICIT |
| 46 | Env | 533 | 606 | ASMTLTVQARLLLSGIVQQQSNLLRAIEAQQHMLQLTVWGIKQLQARILAVER |
| | | | | YLRDQQLLGIWGCSGRLICIT |
| 47 | Env | 536 | 556 | TLTVQARQLLSGIVQQQNNLL |
| 48 | Env | 536 | 556 | TLTVQARLLLSGIVQQQSNLL |
| 49 | Env | 554 | 564 | NLLRAIEAQQH |
| 50 | Env | 554 | 564 | NLLKAIEAQQH |
| 51 | Env | 558 | 584 | AIEAQQHLLQLTVWGIKQLQARVLAVE |
| 52 | Env | 558 | 584 | AIEAQQHMLQLTVWGIKQLQARILAVE |
| 53 | Env | 584 | 592 | ERYLKDQQL |
| 54 | Env | 584 | 592 | ERYLRDQQL |
| 55 | Env | 586 | 594 | YLKDQQLLG |
| 56 | Env | 586 | 594 | YLRDQQLLG |
| 57 | Env | 586 | 610 | YLKDQQLLGIWGCSGKLICTTAVPW |
| 338 | Env | 586 | 610 | YLRDQQLLGLWGCSGKLICPTAVPW |
| 58 | Env | 589 | 606 | DQQLLGIWGCSGKLICTT |
| 59 | Env | 589 | 606 | DQQLLGLWGCSGKLICPT |
| 60 | Env | 594 | 602 | GIWGCSGKL |
| 61 | Env | 594 | 602 | GLWGCSGKL |
| 62 | Env | 678 | 688 | WLWYIKIFIMI |
| 63 | Env | 678 | 688 | WLWYIRIFIMI |
| 64 | Env | 684 | 697 | IFIMIVGGLIGLRI |
| 65 | Env | 684 | 697 | LFIMIVGGLVGLRI |
| 66 | Env | 705 | 719 | VNRVRQGYSPLSFQT |
| 67 | Env | 705 | 719 | VNRVRKGYSPLSFQI |
| 68 | Gag | 1 | 11 | MGARASVLSGG |
| 69 | Gag | 1 | 11 | MGARASILSGG |
| 70 | Gag | 1 | 53 | MGARASVLSGGELDRWEKIRLRPGGKKKYRLKHIVWASRELERFAVNPGLLET |
| 71 | Gag | 1 | 53 | MGARASILSGGKLDKWEKIRLRPGGRKKYKLKHIVWASRELERFAVNPGLLET |
| 72 | Gag | 13 | 25 | LDRWEKIRLRPGG |
| 73 | Gag | 13 | 25 | LDKWEKIRLRPMG |
| 74 | Gag | 19 | 27 | IRLRPGGKK |
| 75 | Gag | 19 | 27 | IRLRPGGRK |
| 76 | Gag | 31 | 53 | LKHIVWASRELERFAVNPGLLET |
| 77 | Gag | 31 | 53 | LKHLVWASRELERFALNPGLLET |
| 78 | Gag | 37 | 51 | ASRELERFAVNPGLL |
| 79 | Gag | 37 | 51 | ASRELERFALNPGLL |
| 80 | Gag | 70 | 78 | TGSEELKSL |
| 81 | Gag | 70 | 78 | TGSEELRSL |
| 82 | Gag | 96 | 104 | DTKEALDKI |
| 83 | Gag | 96 | 104 | DTKEALEKI |
| 84 | Gag | 99 | 107 | EALDKIEEE |
| 85 | Gag | 99 | 107 | EALEKIEEE |
| 86 | Gag | 128 | 137 | VSQNYPIVQN |
| 87 | Gag | 128 | 137 | VSQNFPIVQN |
| 88 | Gag | 133 | 363 | PIVQNLQGQMVHQAISPRTLNAWVKVVEEKAFSPEVIPMFSALSEGATPQDLN |
| | | | | TMLNTVGGHQAAMQMLKETINEEAAEWDRLHPVHAGPIAPGQMREPRGSDIAG |
| | | | | TTSTLQEQIGWMTNNPPIPVGEIYKRWIILGLNKIVRMYSPTSILDIRQGPKE |
| | | | | PERDYVDREYKTLRAEQASQEVKNWMTETLLVQNANPDCKTILKALGPAATLE |
| | | | | EMMTACQGVGGPGHKARVL |
| 89 | Gag | 133 | 363 | PIVQNIQGQMVHQPISPRTLNAWVKVIEEKAFSPEVIPMFTALSEGATPHDLN |
| | | | | TMLNTIGGHQAAMQMLKDTINEEAAEWDRVHPVHAGPVAPGQMRDPRGSDIAG |
| | | | | TTSNLQEQIGWMTSNPPIPVGDIYKRWIIMGLNKIVRMYSPVSILDIKQGPKE |
| | | | | PFRDYVDRFYRTLRAEQASQDVKNWMTETLLVQNSNPDCKTILKALGPGATLE |
| | | | | EMMSACQGVGGPSHKARVL |
| 90 | Gag | 142 | 166 | MVHQAISPRTLNAWVKVVEEKAFSP |
| 91 | Gag | 142 | 166 | MVHQPISPRTLNAWVKVIEEKAFSP |
| 92 | Gag | 147 | 217 | ISPRTLNAWVKVVEEKAFSPEVIPMFSALSEGATPQDLNTMLNTVGGHQAAMQ |
| | | | | MLKETINEEAAEWDRLHP |
| 93 | Gag | 147 | 217 | LSPRTLNAWVKVIEEKAFSPEVIPMFTALSEGATPHDLNTMLNTIGGHQAAMQ |
| | | | | MLKDTINEEAAEWDRVHP |
| 94 | Gag | 147 | 369 | ISPRTLNAWVKVVEEKAFSPEVIPMFSALSEGATPQDLNTMLNTVGGHQAAMQ |
| | | | | MLKETINEEAAEWDRLHPVHAGPIAPGQMREPRGSDIAGTTSTLQEQIGWMTN |
| | | | | NPPIPVGEIYKRWIILGLNKIVRMYSPTSILDIRQGPKEPFRDYVDREYKTLR |
| | | | | AEQASQEVKNWMTETLLVQNANPDCKTILKALGPAATLEEMMTACQGVGGPGH |
| | | | | KARVLAEAMSQ |
| 95 | Gag | 147 | 369 | LSPRTLNAWVKVIEEKAFSPEVIPMFTALSEGATPHDLNTMLNTIGGHQAAMQ |
| | | | | MLKDTINEEAAEWDRVHPVHAGPVAPGQMRDPRGSDIAGSTSTLQEQIAWMTN |
| | | | | NPPIPVGDIYKRWIIMGLNKIVRMYSPVSILDIKQGPKEPFRDYVDRFYRTLR |
| | | | | AEQASQDVKNWMTETLLVQNSNPDCKTILKALGPGATLEEMMSACQGVGGPSH |
| | | | | KARVLAEAMCQ |
| 96 | Gag | 150 | 158 | RTLNAWVKV |
| 97 | Gag | 175 | 199 | LSEGATPQDLNTMLNTVGGHQAAMQ |
| 98 | Gag | 175 | 199 | LSEGATPHDLNTMLNTIGGHQAAMQ |
| 99 | Gag | 183 | 191 | DLNTMLNTV |
| 100 | Gag | 183 | 191 | DLNTMLNTI |
| 101 | Gag | 225 | 251 | PGQMREPRGSDIAGTTSTLQEQIGWMT |
| 102 | Gag | 225 | 251 | PGQMRDPRGSDIAGSTSTLQEQIAWMT |
| 103 | Gag | 253 | 285 | NPPIPVGEIYKRWIILGLNKIVRMYSPTSILDI |
| 104 | Gag | 253 | 285 | NPPIPVGDIYKRWIIMGLNKIVRMYSPVSILDI |
| 339 | Gag | 257 | 282 | PVGEIYKRWIILGLNKIVRMYSPTSI |
| 340 | Gag | 257 | 282 | PVGDIYKRWIIMGLNKIVRMYSPVSI |
| 105 | Gag | 257 | 290 | PVGEIYKRWIILGLNKIVRMYSPTSILDIRQGPK |
| 106 | Gag | 257 | 290 | PVGDIYKRWIIMGLNKIVRMYSPVSILDIKQGPK |
| 107 | Gag | 265 | 282 | WIILGLNKIVRMYSPTSI |
| 108 | Gag | 265 | 282 | WIIMGLNKIVRMYSPVSI |
| 109 | Gag | 281 | 314 | SILDIRQGPKEPFRDYVDRFYKTLRAEQASQEVK |
| 110 | Gag | 281 | 314 | SILDIKQGPKEPFRDYVDRFYRTLRAEQASQDVK |
| 341 | Gag | 288 | 313 | GPKEPFRDYVDRFYKTLRAEQASQEV |
| 342 | Gag | 288 | 313 | GPKEPFRDYVDRFYRTLRAEQASQDV |
| 111 | Gag | 288 | 321 | GPKEPFRDYVDRFYKTLRAEQASQEVKNWMTETL |
| 112 | Gag | 288 | 321 | GPKEPFRDYVDRFYRTLRAEQASQDVKNWMTETL |
| 113 | Gag | 296 | 313 | YVDRFYKTLRAEQASQEV |
| 114 | Gag | 296 | 313 | YVDRFYRTLRAEQASQDV |
| 115 | Gag | 311 | 369 | QEVKNWMTETLLVQNANPDCKTILKALGPAATLEEMMTACQGVGGPGHKARVL |
| | | | | AEAMSQ |
| 116 | Gag | 311 | 369 | QDVKNWMTETLLVQNSNPDCKTILKALGPGATLEEMMSACQGVGGPSHKARVL |
| | | | | AEAMCQ |
| 117 | Gag | 333 | 357 | ILKALGPAATLEEMMTACQGVGGPG |
| 118 | Gag | 333 | 357 | ILKALGPGATLEEMMSACQGVGGPS |
| 119 | Gag | 337 | 361 | LGPAATLEEMMTACQGVGGPGHKAR |
| 120 | Gag | 337 | 361 | LGPGATLEEMMSACQGVGGPSHKAR |
| 121 | Gag | 341 | 349 | ATLEEMMTA |
| 122 | Gag | 341 | 349 | ATLEEMMSA |
| 123 | Gag | 345 | 353 | EMMTACQGV |
| 124 | Gag | 345 | 353 | EMMSACQGV |
| 125 | Gag | 391 | 400 | KCFNCGKEGH |
| 126 | Gag | 391 | 400 | KCFNCGREGH |
| 127 | Gag | 402 | 410 | ARNCRAPRK |
| 128 | Gag | 402 | 410 | AKNCRAPRK |
| 129 | Gag | 402 | 440 | ARNCRAPRKKGCWKCGKEGHQMKDCTERQANFLGKIWPS |
| 130 | Gag | 402 | 440 | AKNCRAPRKRGCWKCGREGHQMKDCNERQANFLGKVWPS |
| 131 | Gag | 404 | 417 | NCRAPRKKGCWKCG |
| 132 | Gag | 404 | 417 | NCRAPRKRGCWKCG |
| 133 | Gag | 412 | 430 | GCWKCGKEGHQMKDCTERQ |
| 134 | Gag | 412 | 430 | GCWKCGREGHQMKDCNERQ |
| 135 | Gag | 424 | 440 | KDCTERQANFLGKIWPS |
| 136 | Gag | 424 | 440 | KDCNERQANFLGKVWPS |
| 137 | Gag | 429 | 444 | RQANFLGKIWPSHKGR |
| 138 | Gag | 429 | 444 | RQANFLGKVWPSHNGR |
| 139 | Gag | 442 | 453 | KGRPGNFLQSRP |
| 140 | Gag | 442 | 453 | NGRPGNFLQNRP |
| 141 | Gag | 488 | 497 | SLRSLFGNDP |
| 142 | Gag | 488 | 497 | SLKSLFGNDP |
| 143 | Gag | 491 | 499 | SLFGNDPSS |
| 144 | Gag | 491 | 499 | SLFGNDPLS |
| 145 | Gag | | | LKHIVWASRELERFAVNPGLLETVSQNYPIVQN |
| 146 | Gag | | | LKHLVWASRELERFALNPGLLETVSQNFPIVQN |
| 147 | Nef | 29 | 37 | GVGAVSRDL |
| 148 | Nef | 29 | 37 | GVGAASRDL |
| 149 | Nef | 64 | 82 | EEVGFPVRPQVPLRPMTYK |
| 150 | Nef | 64 | 82 | EEVGFPVKPQVPLRPMTFK |
| 151 | Nef | 64 | 99 | EEVGFPVKPQVPLRPMTFKGALDLSHFLREKGGLEG |
| 152 | Nef | 64 | 99 | EEVGFPVRPQVPLRPMTYKGALDLSHFLKEKGGLEG |
| 153 | Nef | 81 | 102 | YKAAVDLSHFLREKGGLEGAAY |
| 154 | Nef | 81 | 102 | YKGALDLSHFLKEKGGLEGAAY |
| 155 | Nef | 88 | 97 | SHFLKEKGGL |
| 156 | Nef | 88 | 97 | SHFLREKGGL |
| 157 | Nef | 91 | 99 | LKEKGGLEG |
| 158 | Nef | 91 | 99 | LREKGGLEG |
| 159 | Nef | 117 | 132 | TQGYFPDWQNYTPGPG |
| 160 | Nef | 117 | 132 | TQGFFPDWQNYTPEPG |
| 161 | Nef | 117 | 148 | TQGFFPDWQNYTPEPGIRFPLTFGWCFKLVPL |
| 162 | Nef | 117 | 148 | TQGYFPDWQNYTPGPGTRYPLTFGWCFKLVPV |
| 163 | Nef | 130 | 148 | EPGIRFPLTFGWCFKLVPL |
| 164 | Nef | 130 | 148 | GPGTRYPLTFGWCFKLVPV |
| 165 | Nef | 130 | 154 | GPGIRYPLLTFGWCFKLPVEPEKVE |
| 166 | Nef | 134 | 142 | RYPLTFGWC |
| 167 | Nef | 134 | 142 | RFPLTFGWC |
| 168 | Nef | 134 | 148 | RYPLTFGWCFKLVPV |
| 169 | Nef | 134 | 148 | RFPLTFGWCFKLVPL |
| 170 | Nef | 136 | 148 | PLTFGWCFKLVPV |
| 171 | Nef | 136 | 148 | PLCFGWCFKLVPL |
| 172 | Nef | 137 | 145 | LTFGWCFKL |
| 173 | Nef | 137 | 145 | LCFGWCFKL |
| 174 | Pol | 56 | 67 | FPQITLWQRPLV |
| 175 | Pol | 56 | 67 | LPQITLWQRPIV |
| 176 | Pol | 56 | 117 | FPQITLWQRPLVTIKIGGQLKEALLDTGADDTVLEEMNLPGRWKPKMIGGIG |
| | | | | GFIKVRQYDQ |
| 177 | Pol | 56 | 117 | LPQITLWQRPIVTIKIGGQIKEALLDTGADDTVLEDMNLPGKWKPKMIGGIG |
| | | | | GFIKVKQYDQ |
| 178 | Pol | 72 | 91 | GGQLKEALLDTGADDTVLEE |
| 179 | Pol | 72 | 91 | GGQIKEALLDTGADDTVLED |
| 180 | Pol | 94 | 117 | LPGRWKPKMIGGIGGFIKVRQYDQ |
| 181 | Pol | 94 | 117 | LPGKWKPKMIGGIGGFIKVKQYDQ |
| 182 | Pol | 129 | 260 | GTVLVGPTPVNIIGRNLLTQIGCTLNFPISPIETVPVKLKPGMDGPKVKQWP |
| | | | | LTEEKIKALVEICTEMEKEGKISKIGPENPYNTPVFAIKKKDSTKWRKLVDF |
| | | | | RELNKRTQDFWEVQLGIPHPAGLKKKKS |
| 183 | Pol | 129 | 260 | GTVLIGPTPVNIIGRNLLTQLGCTLNFPISPIDTVPVKLKPGMDGPRVKQWP |
| | | | | LTEEKIKALIEICTEMEKEGKISRIGPENPYNTPIFAIKKKDGTKWRKLVDF |
| | | | | RELNKKTQDFWEVQLGIPHPSGLKKKKS |
| 184 | Pol | 129 | 277 | GTVLVGPTPVNIIGRNLLTQIGCTLNFPISPIETVPVKLKPGMDGPKVKQWP |
| | | | | LTEEKIKALVEICTEMEKEGKISKIGPENPYNTPVFAIKKKDSTKWRKLVDF |
| | | | | RELNKRTQDFWEVQLGIPHPAGLKKKKSVTVLDVGDAYFSVPLDK |
| 185 | Pol | 129 | 277 | GTVLIGPTPVNIIGRNLLTQLGCTLNFPISPIDTVPVKLKPGMDGPRVKQWP |
| | | | | LTEEKIKALIEICTEMEKEGKISRIGPENPYNTPIFAIKKKDGTKWRKLVDF |
| | | | | RELNKKTQDFWEVQLGIPHPSGLKKKKSVTILDVGDAYFSIPLDK |
| 186 | Pol | 129 | 289 | GTVLVGPTPVNIIGRNLLTQIGCTLNFPISPIETVPVKLKPGMDGPKVKQWP |
| | | | | LTEEKIKALVEICTEMEKEGKISKIGPENPYNTPVFAIKKKDSTKWRKLVDF |
| | | | | RELNKRTQDFWEVQLGIPHPAGLKKKKSVTVLDVGDAYFSVPLDKDFRKYTA |
| | | | | FTIPS |
| 187 | Pol | 129 | 289 | GTVLIGPTPVNIIGRNLLTQLGCTLNFPISPIDTVPVKLKPGMDGPRVKQWP |
| | | | | LTEEKIKALIEICTEMEKEGKISRIGPENPYNTPIFAIKKKDGTKWRKLVDF |
| | | | | RELNKKTQDFWEVQLGIPHPSGLKKKKSVTVLDIGDAYFSVPLDKEFRKYTA |
| | | | | FTVPS |
| 188 | Pol | 129 | 320 | GTVLVGPTPVNIIGRNLLTQIGCTLNFPISPIETVPVKLKPGMDGPKVKQWP |
| | | | | LTEEKIKALVEICTEMEKEGKISKIGPENPYNTPVFAIKKKDSTKWRKLVDF |
| | | | | RELNKRTQDFWEVQLGIPHPAGLKKKKSVTVLDVGDAYFSVPLDKDFRKYTA |
| | | | | FTIPSINNETPGIRYQYNVLPQGWKGSPAIFQSSMT |
| 189 | Pol | 129 | 320 | GTVLIGPTPVNIIGRNLLTQLGCTLNFPISPIDTVPVKLKPGMDGPRVKQWP |
| | | | | LTEEKIKALIEICTEMEKEGKISRIGPENPYNTPIFAIKKKDGTKWRKLVDF |
| | | | | RELNKKTQDFWEVQLGIPHPSGLKKKKSVTVLDIGDAYFSVPLDKEFRKYTA |
| | | | | FTVPSTNNETPGVRYQYNVLPMGWKGSPAIFQCSMT |
| 190 | Pol | 144 | 168 | NLLTQIGCTLNFPISPIETVPVKLK |
| 191 | Pol | 144 | 168 | NLLTQLGCTLNFPISPIDTVPVKLK |
| 192 | Pol | 152 | 160 | TLNFPISPI |
| 193 | Pol | 254 | 277 | GLKKKKSVTVLDVGDAYFSVPLDK |
| 194 | Pol | 254 | 277 | GLKKNKSVTVLDVGDAYFSIPLDK |
| 195 | Pol | 278 | 289 | DFRKYTAFTIPS |
| 196 | Pol | 278 | 289 | EFRKYTAFTVPS |
| 197 | Pol | 291 | 315 | NNETPGIRYQYNVLPQGWKGSPAIF |
| 198 | Pol | 291 | 315 | NNETPGVRYQYNVLPMGWKGSPAIF |
| 199 | Pol | 291 | 320 | NNETPGIRYQYNVLPQGWKGSPAIFQSSMT |
| 200 | Pol | 291 | 320 | NNETPGVRYQYNVLPMGWKGSPAIFQCSMT |
| 201 | Pol | 299 | 307 | YQYNVLPQG |
| 202 | Pol | 299 | 307 | YQYNVLPMG |
| 203 | Pol | 315 | 323 | FQSSMTKIL |
| 204 | Pol | 315 | 323 | FQCSMTKIL |
| 205 | Pol | 318 | 327 | SMTKILEPFR |
| 206 | Pol | 318 | 327 | SMTKILDPFR |
| 207 | Pol | 322 | 330 | ILEPFRKQN |
| 208 | Pol | 322 | 330 | ILDPFRKQN |
| 209 | Pol | 326 | 350 | FRKQNPDIVIYQYMDDLYVGSDLEI |
| 343 | Pol | 326 | 350 | FRKQNPDIVIYQYVDDLYVGSDLEI |
| 210 | Pol | 328 | 352 | KQNPDIVIYQYMDDLYVGSDLEIGQ |
| 344 | Pol | 328 | 352 | KQNPDIVIYQYVDDLYVGSDLEIEQ |
| 211 | Pol | 330 | 354 | NPDIVIYQYMDDLYVGSDLEIGQHR |
| 212 | Pol | 330 | 354 | NPDIVIYQYVDDLYVGSDLEIEQHR |
| 213 | Pol | 333 | 354 | IVIYQYMDDLYVGSDLEIGQHR |
| 214 | Pol | 333 | 354 | IVIYQYVDDLYVGSDLEIEQHR |
| 215 | Pol | 334 | 342 | VIYQYMDDL |
| 216 | Pol | 334 | 342 | VIYQYVDDL |
| 217 | Pol | 336 | 344 | YQYMDDLYV |
| 218 | Pol | 336 | 344 | YQYVDDLYV |
| 219 | Pol | 338 | 346 | YMDDLYVGS |
| 220 | Pol | 338 | 346 | YVDDLYVGS |
| 221 | Pol | 367 | 399 | WGFTTPDKKHQKEPPFLWMGYELHPDKWTVQPI |
| 222 | Pol | 367 | 399 | WGLTTPDKKHQKDPPFLWMGYELHPDRWTVQPI |
| 223 | Pol | 367 | 431 | WGFTTPDKKHQKEPPFLWMGYELHPDKWTVQPIVLPEKDSWTVNDIQKLVGK |
| | | | | LNWASQIYPGIKV |
| 224 | Pol | 367 | 431 | WGLTTPDKKHQKDPPFLWMGYELHPDRWTVQPIELPEKESWTVNDIQKLIGK |
| | | | | LNWASQIYAGIKV |
| 225 | Pol | 374 | 398 | KKHQKEPPFLWMGYELHPDKWTVQP |
| 226 | Pol | 374 | 398 | KKHQKDPPFLWMGYELHPDRWTVQP |
| 227 | Pol | 380 | 404 | PPFLWMGYELHPDKWTVQPIVLPEK |
| 228 | Pol | 380 | 404 | PPFLWMGYELHPDRWTVQPIELPEK |
| 229 | Pol | 382 | 390 | FLWMGYELH |
| 230 | Pol | 388 | 396 | ELHPDKWTV |
| 231 | Pol | 388 | 396 | ELHPDRWTV |
| 232 | Pol | 399 | 423 | IVLPEKDSWTVNDIQKLVGKLNWAS |
| 233 | Pol | 399 | 423 | IELPEKESWTVNDIQKLIGKLNWAS |
| 234 | Pol | 400 | 424 | VLPEKDSWTVNDIQKLVGKLNWASQ |
| 235 | Pol | 400 | 424 | ELPEKESWTVNDIQKLIGKLNWASQ |
| 236 | Pol | 401 | 431 | LPEKDSWTVNDIQKLVGKLNWASQIYPGIKV |
| 237 | Pol | 401 | 431 | LPEKESWTVNDIQKLIGKLNWASQIYAGIKV |
| 238 | Pol | 406 | 430 | SWTVNDIQKLVGKLNWASQIYPGIK |
| 239 | Pol | 406 | 430 | SWTVNDIQKLIGKLNWASQIYAGIK |
| 240 | Pol | 407 | 415 | WTVNDIQKL |
| 241 | Pol | 408 | 416 | TVNDIQKLV |
| 242 | Pol | 408 | 416 | TVNDIQKLI |
| 243 | Pol | 414 | 422 | KLVGKLNWA |
| 244 | Pol | 414 | 422 | KLIGKLNWA |
| 245 | Pol | 434 | 442 | LCKLLRGTK |
| 246 | Pol | 434 | 442 | LCKLLRGAK |
| 247 | Pol | 453 | 471 | EAELELAENREILKEPVHG |
| 248 | Pol | 453 | 471 | EAEIELAENREILREPVHG |
| 249 | Pol | 467 | 478 | EPVHGVYYDPSK |
| 250 | Pol | 467 | 478 | EPVHGAYYDPSK |
| 251 | Pol | 490 | 511 | GQWTYQIYQEPFKNLKTGKYAR |
| 252 | Pol | 490 | 511 | GQWSYQIYQEPYKNLKTGKYAK |
| 253 | Pol | 515 | 530 | AHTNDVKQLTEAVQKI |
| 254 | Pol | 515 | 530 | AHTNDVRQLTEAVQKV |
| 255 | Pol | 535 | 544 | IVIWGKTPKF |
| 256 | Pol | 535 | 544 | IVIWGKIPKF |
| 257 | Pol | 542 | 554 | PKFKLPIQKETWE |
| 258 | Pol | 542 | 554 | PKFRLPIQKETWD |
| 259 | Pol | 542 | 606 | PKFKLPIQKETWETWWTEYWQATWIPEWEFVNTPPLVKLWYQLEKEPIVGAE |
| | | | | TFYVDGAANRETK |
| 260 | Pol | 542 | 606 | PKFRLPIQKETWDTWWTDYWQATWIPEWEFTNTPPLVKLWYQLETEPIAGVE |
| | | | | TFYVDGASNRETK |
| 261 | Pol | 553 | 577 | WETWWTEYWQATWIPEWEFVNTPPL |
| 262 | Pol | 553 | 577 | WDTWWTDYWQATWIPEWEFTNTPPL |
| 263 | Pol | 559 | 589 | EYWQATWIPEWEFVNTPPLVKLWYQLEKEPI |
| 264 | Pol | 559 | 589 | DYWQATWIPEWEFTNTPPLVKLWYQLETEPI |
| 265 | Pol | 561 | 569 | WQATWIPEW |
| 266 | Pol | 591 | 606 | GAETFYVDGAANRETK |
| 267 | Pol | 591 | 606 | GVETFYVDGASNRETK |
| 268 | Pol | 625 | 637 | TDTTNQKTELQAI |
| 269 | Pol | 625 | 637 | ADTTNQKTELHAI |
| 270 | Pol | 636 | 644 | AIHLALQDS |
| 271 | Pol | 636 | 644 | AIYLALQDS |
| 272 | Pol | 639 | 671 | LALQDSGLEVNIVTDSQYALGIIQAQPDKSESE |
| 273 | Pol | 639 | 671 | LALQDSGSEVNIVTDSQYAIGIIQAQPDRSESE |
| 274 | Pol | 642 | 666 | QDSGLEVNIVTDSQYALGIIQAQPD |
| 275 | Pol | 642 | 666 | QDSGSEVNIVTDSQYAIGIIQAQPD |
| 276 | Pol | 650 | 658 | IVTDSQYAL |
| 277 | Pol | 650 | 658 | IVTDSQYAI |
| 278 | Pol | 673 | 681 | VSQIIEQLI |
| 279 | Pol | 673 | 681 | VNQIIEQLI |
| 280 | Pol | 675 | 684 | QIIEQLIKKE |
| 281 | Pol | 675 | 684 | QIIEQLINKE |
| 282 | Pol | 683 | 708 | KEKVYLAWVPAHKGIGGNEQVDKLVS |
| 283 | Pol | 683 | 708 | KEKIYLAWVPAHKGIGGNEQIDKLVS |
| 284 | Pol | 710 | 725 | GIRKVLFLDGIDKAQE |
| 285 | Pol | 710 | 725 | GIRRVLFLDGIEKAQD |
| 286 | Pol | 727 | 735 | HEKYHSNWR |
| 287 | Pol | 727 | 735 | HEKYHNNWR |
| 288 | Pol | 737 | 745 | MASDFNLPP |
| 289 | Pol | 737 | 745 | MASDFNIPP |
| 290 | Pol | 741 | 753 | FNLPPVVAKEIVA |
| 291 | Pol | 741 | 753 | FNLPPIVAKEIVA |
| 292 | Pol | 741 | 827 | FNLPPVVAKEIVASCDKCQLKGEAMHGQVDCSPGIWQLDCTHLEGKIILVAV |
| | | | | HVASGYIEAEVIPAETGQETAYFLLKLAGRWPVKT |
| 293 | Pol | 741 | 827 | FNLPPIVAKEIVACCDKCQLKGEAIHGQVDCSPGVWQLDCTHLEGKVILVAV |
| | | | | HVASGYIEAEIIPTETGQETAYFILKLAGRWPVTT |
| 294 | Pol | 747 | 827 | VAKEIVASCDKCQLKGEAMHGQVDCSPGIWQLDCTHLEGKIILVAVHVASGY |
| | | | | IEAEVIPAETGQETAYFLLKLAGRWPVKT |
| 295 | Pol | 747 | 827 | VAKEIVACCDKCQLKGEAIHGQVDCSPGVWQLDCTHLEGKVILVAVHVASGY |
| | | | | MEAEVIPTETGQETAYFILKLAGRWPVTT |
| 296 | Pol | 759 | 783 | QLKGEAMHGQVDCSPGIWQLDCTHL |
| 297 | Pol | 759 | 783 | QLKGEAIHGQVDCSPGVWQLDCTHL |
| 298 | Pol | 767 | 775 | GQVDCSPGI |
| 299 | Pol | 767 | 775 | GQVDCSPGV |
| 300 | Pol | 768 | 792 | QVDCSPGIWQLDCTHLEGKIILVAV |
| 301 | Pol | 768 | 792 | QVDCSPGVWQLDCTHLEGKVILVAV |
| 302 | Pol | 776 | 784 | WQLDCTHLE |
| 303 | Pol | 834 | 858 | SNFTSTTVKAACWWAGIKQEFGIPY |
| 304 | Pol | 834 | 858 | SNFTSTAVKAACWWAGVKQEFGIPY |
| 305 | Pol | 840 | 919 | TVKAACWWAGIKQEFGIPYNPQSQGVVESMNKELKKIIGQVRDQAEHLKTAV |
| | | | | QMAVFIHNFKRKGGIGGYSAGERIVDII |
| 306 | Pol | 840 | 919 | AVKAACWWAGVKQEFGIPYHPQSQGVVESMNNELKKIIGQIRDQAEQLKTAV |
| | | | | QMAVLIHNFKRKGGIGEYSAGERIIDII |
| 307 | Pol | 840 | 920 | TVKAACWWAGIKQEFGIPYNPQSQGVVESMNKELKKIIGQVRDQAEHLKTAV |
| | | | | QMAVFIHNFKRKGGIGGYSAGERIVDIIA |
| 308 | Pol | 840 | 920 | AVKAACWWAGVKQEFGIPYHPQSQGVVESMNNELKKIIGQIRDQAEQLKTAV |
| | | | | QMAVLIHNFKRKGGIGEYSAGERIIDIIA |
| 309 | Pol | 840 | 1003 | TVKAACWWAGIKQEFGIPYNPQSQGVVESMNKELKKIIGQVRDQAEHLKTAV |
| | | | | QMAVFIHNFKRKGGIGGYSAGERIVDIIATDIQTKELQKQIIKIQNFRVYYR |
| | | | | DSRDPLWKGPAKLLWKGEGAVVIQDNSDIKVVPRRKAKIIRDYGKQMAGDDC |
| | | | | VASRQDED |
| 310 | Pol | 840 | 1003 | AVKAACWWAGVKQEFGIPYNTQSQGVVESMNNELKKIIGQIRDQAEHLKTAV |
| | | | | QMAVLIHNFKRKGGIGEYSAGERIIDIIATDIQTRELQKQIIKLQNFRVYYR |
| | | | | DNRDPLWKGPARLLWKGEGAVVIQDNSEIKVVPRRKVKIIRDYGKRMAGDDC |
| | | | | VAGRQDED |
| 311 | Pol | 842 | 850 | KAACWWAGI |
| 312 | Pol | 842 | 850 | KAACWWAGV |
| 313 | Pol | 917 | 925 | DIIATDIQT |
| 314 | Pol | 917 | 925 | DIIASDIQT |
| 315 | Pol | 922 | 930 | DIQTKELQK |
| 316 | Pol | 922 | 930 | DIQTRELQK |
| 317 | Pol | 924 | 932 | QTKELQKQI |
| 318 | Pol | 924 | 932 | QTRELQKQI |
| 319 | Pol | 931 | 1003 | AITKIQNFRVYYRDSRDPLWKGPAKLLWKGEGAVVIQDNSDIKVVPRRKAKI |
| | | | | IRDYGKQMAGDDCVASRQDED |
| 320 | Pol | 931 | 1003 | AITKLQNFRVYYRDNRDPLWKGPARLLWKGEGAVVIQDNSEIKVVPRRKVKI |
| | | | | IRDYGKRMAGDDCVAGRQDED |
| 321 | Pol | 932 | 1003 | ITKIQNFRVYYRDSRDPLWKGPAKLLWKGEGAVVIQDNSDIKVVPRRKAKII |
| | | | | RDYGKQMAGDDCVASRQDED |
| 322 | Pol | 932 | 1003 | ITKLQNFRVYYRDNRDPLWKGPARLLWKGEGAVVIQDNSEIKVVPRRKVKII |
| | | | | RDYGKRMAGDDCVAGRQDED |
| 323 | Pol | 940 | 964 | VYYRDSRDPLWKGPAKLLWKGEGAV |
| 324 | Pol | 940 | 964 | VYYRDNRDPLWKGPARLLWKGEGAV |
| 325 | Pol | 947 | 971 | DPLWKGPAKLLWKGEGAVVIQDNSD |
| 326 | Pol | 947 | 971 | DPLWKGPARLLWKGEGAVVIQDNSE |
| 327 | Pol | 948 | 956 | PLWKGPAKL |
| 328 | Pol | 948 | 956 | PLWKGPARL |
| 329 | Pol | 948 | 972 | PLWKGPAKLLWKGEGAVVIQDNSDI |
| 330 | Pol | 948 | 972 | PLWKGPARLLWKGEGAVVIQDNSEI |
| 331 | Pol | 955 | 963 | KLLWKGEGA |
| 332 | Pol | 955 | 963 | RLLWKGEGA |
| 333 | Pol | 956 | 964 | LLWKGEGAV |
| 334 | Pol | 980 | 1003 | AKIIRDYGKQMAGDDCVASRQDED |
| 335 | Pol | 980 | 1003 | VKIIRDYGKRMAGDDCVAGRQDED |
| 336 | Pol | 988 | 996 | KQMAGDDCV |
| 337 | Pol | 988 | 996 | KRMAGDDCV |

TABLE C
polypeptide segments in conserved regions of HIV-1 proteins

| SEQ ID | | | | | |
|---|---|---|---|---|---|
| NO: | Gene | Start | End | Length | Sequence |
| 2 | Env | 34 | 48 | 15 | LWVTVYYGVPVWKEA |
| 3 | Env | 34 | 48 | 15 | LWVTIYYGVPVWKDA |
| 8 | Env | 48 | 61 | 14 | ATTTLFCASDAKAY |
| 9 | Env | 48 | 61 | 14 | ANTTLFCASDAKGY |
| 13 | Env | 66 | 83 | 18 | HNVWATHACVPTDPNPQE |
| 14 | Env | 66 | 83 | 18 | HNIWATHACVPTDPSPQE |
| 17 | Env | 107 | 129 | 23 | DIISLWDQSLKPCVKLTPLCVTL |
| 18 | Env | 107 | 129 | 23 | DIISLWDESLKPCVKLTPICVTL |
| 23 | Env | 209 | 226 | 18 | SFEPIPIHYCAPAGFAIL |
| 24 | Env | 209 | 226 | 18 | TFEPIPIHYCTPAGFAIL |
| 25 | Env | 220 | 228 | 9 | PAGFAILKC |
| 26 | Env | 220 | 228 | 9 | PAGFALLKC |
| 28 | Env | 241 | 268 | 28 | NVSTVQCTHGIRPVVSTQLLLNGSLAEE |
| 29 | Env | 241 | 268 | 28 | NISTVQCTHGIKPVVSTQLLLNGSLAEK |
| 31 | Env | 376 | 386 | 11 | FNCGGEFFYCN |
| 32 | Env | 376 | 386 | 11 | FNCRGEFFYCN |
| 33 | Env | 430 | 439 | 10 | VGKAMYAPPI |
| 34 | Env | 430 | 439 | 10 | VGRAMYAPPI |
| 35 | Env | 472 | 481 | 10 | GGDMRDNWRS |
| 36 | Env | 472 | 481 | 10 | GGNMKDNWRS |
| 37 | Env | 475 | 489 | 15 | MRDNWRSELYKYKVV |
| 38 | Env | 475 | 489 | 15 | MKDNWRSELYRYKVV |
| 39 | Env | 501 | 511 | 11 | AKRRVVQREKR |
| 40 | Env | 501 | 511 | 11 | ARRRVVQREKR |
| 43 | Env | 519 | 534 | 16 | FLGFLGAAGSTMGAAS |
| 44 | Env | 519 | 534 | 16 | FLGFLGTAGSTMGAAA |
| 45 | Env | 533 | 606 | 74 | ASITLTVQARQLLSGIVQQQNNLLRAIEAQQHLLQLTVWGIKQLQARVLAVER |
| | | | | | YLKDQQLLGIWGCSGKLICTT |
| 46 | Env | 533 | 606 | 74 | ASMTLTVQARLLLSGIVQQQSNLLRAIEAQQHMLQLTVWGIKQLQARILAVER |
| | | | | | YLRDQQLLGIWGCSGRLICTT |
| 47 | Env | 536 | 556 | 21 | TLTVQARQLLSGIVQQQNNLL |
| 48 | Env | 536 | 556 | 21 | TLTVQARLLLSGIVQQQSNLL |
| 49 | Env | 554 | 564 | 11 | NLLRAIEAQQH |
| 50 | Env | 554 | 564 | 11 | NLLKAIEAQQH |
| 51 | Env | 558 | 584 | 27 | AIEAQQHLLQLTVWGIKQLQARVLAVE |
| 52 | Env | 558 | 584 | 27 | AIEAQQHMLQLTVWGIKQLQARILAVE |
| 53 | Env | 584 | 592 | 9 | ERYLKDQQL |
| 54 | Env | 584 | 592 | 9 | ERYLRDQQL |
| 55 | Env | 586 | 594 | 9 | YLKDQQLLG |
| 56 | Env | 586 | 594 | 9 | YLRDQQLLG |
| 58 | Env | 589 | 606 | 18 | DQQLLGIWGCSGKLICTT |
| 59 | Env | 589 | 606 | 18 | DQQLLGLWGCSGKLICPT |
| 62 | Env | 678 | 688 | 11 | WLWYIKIFIMI |
| 63 | Env | 678 | 688 | 11 | WLWYIRIFIMI |
| 64 | Env | 684 | 697 | 14 | IFIMIVGGLIGLRI |
| 65 | Env | 684 | 697 | 14 | LFIMIVGGLVGLRI |
| 66 | Env | 705 | 719 | 15 | VNRVRQGYSPLSFQT |
| 67 | Env | 705 | 719 | 15 | VNRVRKGYSPLSFQI |
| 68 | Gag | 1 | 11 | 11 | MGARASVLSGG |
| 69 | Gag | 1 | 11 | 11 | MGARASILSGG |
| 72 | Gag | 13 | 25 | 13 | LDRWEKIRLRPGG |
| 73 | Gag | 13 | 25 | 13 | LDKWEKIRLRPMG |
| 74 | Gag | 19 | 27 | 9 | IRLRPGGKK |
| 75 | Gag | 19 | 27 | 9 | IRLRPGGRK |
| 76 | Gag | 31 | 53 | 23 | LKHIVWASRELERFAVNPGLLET |
| 77 | Gag | 31 | 53 | 23 | LKHLVWASRELERFALNPGLLET |
| 80 | Gag | 70 | 78 | 9 | TGSEELKSL |
| 81 | Gag | 70 | 78 | 9 | TGSEELRSL |
| 82 | Gag | 96 | 104 | 9 | DTKEALDKI |
| 83 | Gag | 96 | 104 | 9 | DTKEALEKI |
| 84 | Gag | 99 | 107 | 9 | EALDKIEEE |
| 85 | Gag | 99 | 107 | 9 | EALEKIEEE |
| 86 | Gag | 128 | 137 | 10 | VSQNYPIVQN |
| 87 | Gag | 128 | 137 | 10 | VSQNFPIVQN |
| 88 | Gag | 133 | 363 | 231 | PIVQNLQGQMVHQAISPRTLNAWVKVVEEKAFSPEVIPMFSALSEGATPQDLN |
| | | | | | TMLNTVGGHQAAMQMLKETINEEAAEWDRLHPVHAGPIAPGQMREPRGSDIAG |
| | | | | | TTSTLQEQIGWMTNNPPIPVGEIYKRWIILGLNKIVRMYSPTSILDIRQGPKE |
| | | | | | PFRDYVDRFYKTLRAEQASQEVKNWMTETLLVQNANPDCKTILKALGPAATLE |
| | | | | | EMMTACQGVGGPGHKARVL |
| 89 | Gag | 133 | 363 | 231 | PIVQNIQGQMVHQPISPRTLNAWVKVIEEKAFSPEVIPMFTALSEGATPHDLN |
| | | | | | TMLNTIGGHQAAMQMLKDTINEEAAEWDRVHPVHAGPVAPGQMRDPRGSDIAG |
| | | | | | TTSNLQEQIGWMTSNPPIPVGDIYKRWIIMGLNKIVRMYSPVSILDIKQGPKE |
| | | | | | PFRDYVDRFYRTLRAEQASQDVKNWMTETLLVQNSNPDCKTILKALGPGATLE |
| | | | | | EMMSACQGVGGPSHKARVL |
| 92 | Gag | 147 | 217 | 71 | ISPRTLNAWVKVVEEKAFSPEVIPMFSALSEGATPQDLNTMLNTVGGHQAAMQ |
| | | | | | MLKETINEEAAEWDRLHP |
| 93 | Gag | 147 | 217 | 71 | LSPRTLNAWVKVIEEKAFSPEVIPMFTALSEGATPHDLNTMLNTIGGHQAAMQ |
| | | | | | MLKDTINEEAAEWDRVHP |
| 101 | Gag | 225 | 251 | 27 | PGQMREPRGSDIAGTTSTLQEQIGWMT |
| 102 | Gag | 225 | 251 | 27 | PGQMRDPRGSDIAGSTSTLQEQIAWMT |
| 103 | Gag | 253 | 285 | 33 | NPPIPVGEIYKRWIILGLNKIVRMYSPTSILDI |
| 104 | Gag | 253 | 285 | 33 | NPPIPVGDIYKRWIIMGLNKIVRMYSPVSILDI |
| 109 | Gag | 281 | 314 | 34 | SILDIRQGPKEPFRDYVDRFYKTLRAEQASQEVK |
| 110 | Gag | 281 | 314 | 34 | SILDIKQGPKEPFRDYVDRFYRTLRAEQASQDVK |
| 115 | Gag | 311 | 369 | 59 | QEVKNWMTETLLVQNANPDCKTILKALGPAATLEEMMTACQGVGGPGHKARVL |
| | | | | | AEAMSQ |
| 116 | Gag | 311 | 369 | 59 | QDVKNWMTEILLVQNSNPDCKTILKALGPGATLEEMMSACQGVGGPSHKARVL |
| | | | | | AEAMCQ |
| 125 | Gag | 391 | 400 | 10 | KCFNCGKEGH |
| 126 | Gag | 391 | 400 | 10 | KCFNCGREGH |
| 127 | Gag | 402 | 410 | 9 | ARNCRAPRK |
| 128 | Gag | 402 | 410 | 9 | AKNCRAPRK |
| 129 | Gag | 402 | 440 | 39 | ARNCRAPRKKGCWKCGKEGHQMKDCTERQANFLGKIWPS |
| 130 | Gag | 402 | 440 | 39 | AKNCRAPRKRGCWKCGREGHQMKDCNERQANFLGKVWPS |
| 131 | Gag | 404 | 417 | 14 | NCRAPRKKGCWKCG |
| 132 | Gag | 404 | 417 | 14 | NCRAPRKRGCWKCG |
| 133 | Gag | 412 | 430 | 19 | GCWKCGKEGHQMKDCTERQ |
| 134 | Gag | 412 | 430 | 19 | GCWKCGREGHQMKDCNERQ |
| 135 | Gag | 424 | 440 | 17 | KDCTERQANFLGKIWPS |
| 136 | Gag | 424 | 440 | 17 | KDCNERQANFLGKVWPS |
| 139 | Gag | 442 | 453 | 12 | KGRPGNFLQSRP |
| 140 | Gag | 442 | 453 | 12 | NGRPGNFLQNRP |
| 141 | Gag | 488 | 497 | 10 | SLRSLFGNDP |
| 142 | Gag | 488 | 497 | 10 | SLKSLFGNDP |
| 143 | Gag | 491 | 499 | 9 | SLFGNDPSS |
| 144 | Gag | 491 | 499 | 9 | SLFGNDPLS |
| 145 | Gag | | | | LKHIVWASRELERFAVNPGLLETVSQNYPIVQN |
| 146 | Gag | | | | LKHLVWASRELERFALNPGLLETVSQNFPIVQN |
| 147 | Nef | 29 | 37 | 9 | GVGAVSRDL |
| 148 | Nef | 29 | 37 | 9 | GVGAASRDL |
| 149 | Nef | 64 | 82 | 19 | EEVGFPVRPQVPLRPMTYK |
| 150 | Nef | 64 | 82 | 19 | EEVGFPVKPQVPLRPMTFK |
| 155 | Nef | 88 | 97 | 10 | SHFLKEKGGL |
| 156 | Nef | 88 | 97 | 10 | SHFLREKGGL |
| 157 | Nef | 91 | 99 | 9 | LKEKGGLEG |
| 158 | Nef | 91 | 99 | 9 | LREKGGLEG |
| 159 | Nef | 117 | 132 | 16 | TQGYFPDWQNYTPGPG |
| 160 | Nef | 117 | 132 | 16 | TQGFFPDWQNYTPEPG |
| 166 | Nef | 134 | 142 | 9 | RYPLTFGWC |
| 167 | Nef | 134 | 142 | 9 | RFPLTFGWC |
| 168 | Nef | 134 | 148 | 15 | RYPLTFGWCFKLVPV |
| 169 | Nef | 134 | 148 | 15 | RFPLTFGWCFKLVPL |
| 170 | Nef | 136 | 148 | 13 | PLTFGWCFKLVPV |
| 171 | Nef | 136 | 148 | 13 | PLCFGWCFKLVPL |
| 174 | Pol | 56 | 67 | 12 | FPQITLWQRPLV |
| 175 | Pol | 56 | 67 | 12 | LPQITLWQRPIV |
| 178 | Pol | 72 | 91 | 20 | GGQLKEALLDTGADDTVLEE |
| 179 | Pol | 72 | 91 | 20 | GGQIKEALLDTGADDTVLED |
| 180 | Pol | 94 | 117 | 24 | LPGRWKPKMIGGIGGFIKVRQYDQ |
| 181 | Pol | 94 | 117 | 24 | LPGKWKPKMIGGIGGFIKVKQYDQ |
| 182 | Pol | 129 | 260 | 132 | GTVLVGPTPVNIIGRNLLTQIGCTLNFPISPIETVPVKLKPGMDGPKVKQWPL |
| | | | | | TEEKIKALVEICTEMEKEGKISKIGPENPYNTPVFAIKKKDSTKWRKLVDFRE |
| | | | | | LNKRIQDFWEVQLGIPHPAGLKKKKS |
| 183 | Pol | 129 | 260 | 132 | GTVLIGPTPVNIIGRNLLTQLGCTLNFPISPIDTVPVKLKPGMDGPRVKQWPL |
| | | | | | TEEKIKALIEICTEMEKEGKISRIGPENPYNTPIFAIKKKDGTKWRKLVDFRE |
| | | | | | LNKKTQDFWEVQLGIPHPSGLKKKKS |
| 184 | Pol | 129 | 277 | 149 | GTVLVGPTPVNIIGRNLLTQIGCTLNFPISPIETVPVKLKPGMDGPKVKQWPL |
| | | | | | TEEKIKALVEICTEMEKEGKISKIGPENPYNTPVFAIKKKDSTKWRKLVDFRE |
| | | | | | LNKRTQDFWEVQLGIPHPAGLKKKKSVTVLDVGDAYFSVPLDK |
| 185 | Pol | 129 | 277 | 149 | GTVLIGPTPVNIIGRNLLTQLGCTLNFPISPIDTVPVKLKPGMDGPRVKQWPL |
| | | | | | TEEKIKALIEICTEMEKEGKISRIGPENPYNTPIFAIKKKDGTKWRKLVDFRE |
| | | | | | LNKKTQDFWEVQLGIPHPSGLKKKKSVTILDVGDAYFSIPLDK |
| 193 | Pol | 254 | 277 | 24 | GLKKKKSVTVLDVGDAYFSVPLDK |
| 194 | Pol | 254 | 277 | 24 | GLKKNKSVTVLDVGDAYFSIPLDK |
| 195 | Pol | 278 | 289 | 12 | DFRKYTAFTIPS |
| 196 | Pol | 278 | 289 | 12 | EFRKYTAFTVPS |
| 197 | Pol | 291 | 315 | 25 | NNETPGIRYQYNVLPQGWKGSPAIF |
| 198 | Pol | 291 | 315 | 25 | NNETPGVRYQYNVLPMGWKGSPAIF |
| 199 | Pol | 291 | 320 | 30 | NNETPGIRYQYNVLPQGWKGSPAIFQSSMT |
| 200 | Pol | 291 | 320 | 30 | NNETPGVRYQYNVLPMGWKGSPAIFQCSMT |
| 203 | Pol | 315 | 323 | 9 | FQSSMTKIL |
| 204 | Pol | 315 | 323 | 9 | FQCSMTKIL |
| 205 | Pol | 318 | 327 | 10 | SMTKILEPFR |
| 206 | Pol | 318 | 327 | 10 | SMTKILDPFR |
| 207 | Pol | 322 | 330 | 9 | ILEPFRKQN |
| 208 | Pol | 322 | 330 | 9 | ILDPFRKQN |
| 213 | Pol | 333 | 354 | 22 | IVIYQYMDDLYVGSDLEIGQHR |
| 214 | Pol | 333 | 354 | 22 | IVIYQYVDDLYVGSDLEIEQHR |
| 221 | Pol | 367 | 399 | 33 | WGFTTPDKKHQKEPPFLWMGYELHPDKWTVQPI |
| 222 | Pol | 367 | 399 | 33 | WGLTTPDKKHQKDPPFLWMGYELHPDRWTVQPI |
| 236 | Pol | 401 | 431 | 31 | LPEKDSWTVNDIQKLVGKLNWASQIYPGIKV |
| 237 | Pol | 401 | 431 | 31 | LPEKESWTVNDIQKLIGKLNWASQIYAGIKV |
| 245 | Pol | 434 | 442 | 9 | LCKLLRGTK |
| 246 | Pol | 434 | 442 | 9 | LCKLLRGAK |
| 247 | Pol | 453 | 471 | 19 | EAELELAENREILKEPVHG |
| 248 | Pol | 453 | 471 | 19 | EAEIELAENREILREPVHG |
| 249 | Pol | 467 | 478 | 12 | EPVHGVYYDPSK |
| 250 | Pol | 467 | 478 | 12 | EPVHGAYYDPSK |
| 251 | Pol | 490 | 511 | 22 | GQWTYQIYQEPFKNLKTGKYAR |
| 252 | Pol | 490 | 511 | 22 | GQWSYQIYQEPYKNLKTGKYAK |
| 253 | Pol | 515 | 530 | 16 | AHTNDVKQLTEAVQKI |
| 254 | Pol | 515 | 530 | 16 | AHTNDVRQLTEAVQKV |
| 255 | Pol | 535 | 544 | 10 | IVIWGKTPKF |
| 256 | Pol | 535 | 544 | 10 | IVIWGKIPKF |
| 257 | Pol | 542 | 554 | 13 | PKFKLPIQKETWE |
| 258 | Pol | 542 | 554 | 13 | PKFRLPIQKETWD |
| 263 | Pol | 559 | 589 | 31 | EYWQATWIPEWEFVNTPPLVKLWYQLEKEPI |
| 264 | Pol | 559 | 589 | 31 | DYWQATWIPEWEFTNTPPLVKLWYQLETEPI |
| 266 | Pol | 591 | 606 | 16 | GAETFYVDGAANRETK |
| 267 | Pol | 591 | 606 | 16 | GVETFYVDGASNRETK |
| 268 | Pol | 625 | 637 | 13 | TDTTNQKTELQAI |
| 269 | Pol | 625 | 637 | 13 | ADTTNQKTELHAI |
| 270 | Pol | 636 | 644 | 9 | AIHLALQDS |
| 271 | Pol | 636 | 644 | 9 | AIYLALQDS |
| 272 | Pol | 639 | 671 | 33 | LALQDSGLEVNIVTDSQYALGIIQAQPDKSESE |
| 273 | Pol | 639 | 671 | 33 | LALQDSGSEVNIVTDSQYAIGIIQAQPDRSESE |
| 278 | Pol | 673 | 681 | 9 | VSQIIEQLI |
| 279 | Pol | 673 | 681 | 9 | VNQIIEQLI |
| 280 | Pol | 675 | 684 | 10 | QIIEQLIKKE |
| 281 | Pol | 675 | 684 | 10 | QIIEQLINKE |
| 282 | Pol | 683 | 708 | 26 | KEKVYLAWVPAHKGIGGNEQVDKLVS |
| 283 | Pol | 683 | 708 | 26 | KEKIYLAWVPAHKGIGGNEQIDKLVS |
| 284 | Pol | 710 | 725 | 16 | GIRKVLFLDGIDKAQE |
| 285 | Pol | 710 | 725 | 16 | GIRRVLFLDGIEKAQD |
| 286 | Pol | 727 | 735 | 9 | HEKYHSNWR |
| 287 | Pol | 727 | 735 | 9 | HEKYHNNWR |
| 288 | Pol | 737 | 745 | 9 | MASDFNLPP |
| 289 | Pol | 737 | 745 | 9 | MASDFNIPP |
| 290 | Pol | 741 | 753 | 13 | FNLPPVVAKEIVA |
| 291 | Pol | 741 | 753 | 13 | FNLPPIVAKEIVA |
| 292 | Pol | 741 | 827 | 87 | FNLPPVVAKEIVASCDKCQLKGEAMHGQVDCSPGIWQLDCTHLEGKIILVAVH |
| | | | | | VASGYIEAEVIPAETGQETAYFLLKLAGRWPVKT |
| 293 | Pol | 741 | 827 | 87 | FNLPPIVAKEIVACCDKCQLKGEAIHGQVDCSPGVWQLDCTHLEGKVILVAVH |
| | | | | | VASGYIEAEIIPTETGQETAYFILKLAGRWPVTT |
| 294 | Pol | 747 | 827 | 81 | VAKEIVASCDKCQLKGEAMHGQVDCSPGIWQLDCTHLEGKIILVAVHVASGYI |
| | | | | | EAEVIPAETGQETAYFLLKLAGRWPVKT |
| 295 | Pol | 747 | 827 | 81 | VAKEIVACCDKCQLKGEAIHGQVDCSPGVWQLDCTHLEGKVILVAVHVASGYM |
| | | | | | EAEVIPTETGQETAYFILKLAGRWPVTT |
| 305 | Pol | 840 | 919 | 80 | TVKAACWWAGIKQEFGIPYNPQSQGVVESMNKELKKIIGQVRDQAEHLKTAVQ |
| | | | | | MAVFIHNFKRKGGIGGYSAGERIVDII |
| 306 | Pol | 840 | 919 | 80 | AVKAACWWAGVKQEFGIPYHPQSQGVVESMNNELKKIIGQIRDQAEQLKTAVQ |
| | | | | | MAVLIHNFKRKGGIGEYSAGERIIDII |
| 307 | Pol | 840 | 920 | 81 | TVKAACWWAGIKQEFGIPYNPQSQGVVESMNKELKKIIGQVRDQAEHLKTAVQ |
| | | | | | MAVFIHNFKRKGGIGGYSAGERIVDIIA |
| 308 | Pol | 840 | 920 | 81 | AVKAACWWAGVKQEFGIPYHPQSQGVVESMNNELKKIIGQIRDQAEQLKTAVQ |
| | | | | | MAVLIHNFKRKGGIGEYSAGERIIDIIA |
| 309 | Pol | 840 | 1003 | 164 | TVKAACWWAGIKQEFGIPYNPQSQGVVESMNKELKKIIGQVRDQAEHLKTAVQ |
| | | | | | MAVFIHNFKRKGGIGGYSAGERIVDIIATDIQTKELQKQIIKIQNFRVYYRDS |
| | | | | | RDPLWKGPAKLLWKGEGAVVIQDNSDIKVVPRRKAKIIRDYGKQMAGDDCVAS |
| | | | | | RQDED |
| 310 | Pol | 840 | 1003 | 164 | AVKAACWWAGVKQEFGIPYNTQSQGVVESMNNELKKIIGQIRDQAEHLKTAVQ |
| | | | | | MAVLIHNFKRKGGIGEYSAGERIIDIIATDIQTRELQKQIIKLQNFRVYYRDN |
| | | | | | RDPLWKGPARLLWKGEGAVVIQDNSEIKVVPRRKVKIIRDYGKRMAGDDCVAG |
| | | | | | RQDED |
| 313 | Pol | 917 | 925 | 9 | DIIATDIQT |
| 314 | Pol | 917 | 925 | 9 | DIIASDIQT |
| 315 | Pol | 922 | 930 | 9 | DIQTKELQK |
| 316 | Pol | 922 | 930 | 9 | DIQTRELQK |
| 317 | Pol | 924 | 932 | 9 | QTKELQKQI |
| 318 | Pol | 924 | 932 | 9 | QTRELQKQI |
| 321 | Pol | 932 | 1003 | 72 | ITKIQNFRVYYRDSRDPLWKGPAKLLWKGEGAVVIQDNSDIKVVPRRKAKIIR |
| | | | | | DYGKQMAGDDCVASRQDED |
| 322 | Pol | 932 | 1003 | 72 | ITKLQNFRVYYRDNRDPLWKGPARLLWKGEGAVVIQDNSEIKVVPRRKVKIIR |
| | | | | | DYGKRMAGDDCVAGRQDED |

With respect to the range of lengths of the individual polypeptide or peptide segments, in various embodiments, each polypeptide segment is at least 8 amino acids in length, and up to about 250 amino acids in length, e.g., from at least 8 amino acids in length up to 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 45, 50, 55, 60, 65, 70, 75, 80, 85, 90, 95, 100, 110, 120, 130, 140, 150, 160, 170, 180, 190, 200, 210, 220, 230, 240 or 250 amino acids in length. In various embodiments, each polypeptide segment is at least 8 amino acids in length, and up to about 35 amino acids in length, e.g., from at least 8 amino acids in length up to 9, 10, 11, 12, 13, 14, 15, 16, 17, 18, 19, 20, 21, 22, 23, 24, 25, 26, 27, 28, 29, 30, 31, 32, 34 or 35 amino acids in length. In various embodiments, each polypeptide segment is at least 15 amino acids in length, and up to about 30 amino acids in length, e.g., from at least 15 amino acids in length up to 16, 17, 18, 19, 20, 21, 22, 23, 24, 25, 26, 27, 28, 29 or 30 amino acids in length.

With respect to the length of the full-length fusion polypeptide, in various embodiments, in some embodiments, the full-length of the fusion polypeptide comprises at least about 350 amino acids and up to about 1000 amino acids, e.g., at least about 350 amino acids and up to about 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 or 1000 amino acids. With respect to the length of the full-length fusion polypeptide, in various embodiments, in some embodiments, the full-length of the fusion polypeptide comprises at least about 350 amino acids and up to about 800 amino acids, e.g., at least about 350 amino acids and up to about 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 670, 680, 690, 700, 710, 720, 730, 740, 750, 760, 770, 780, 790, or 800 amino acids. In some embodiments, the full-length of the fusion polypeptide is no longer than 800 amino acids, e.g., no longer than 795, 790, 785, 780, 775, 770, 765, 760, 755, 750, 745, 740, 735, 730, 725, 720, 715, 710, 705 or 700 amino acids.

Generally, the fusion polypeptides are immunogenic, in that they are capable of eliciting an immune response in a human, e.g., against HIV-1. In some embodiments, the fusion polypeptides, optionally in combination with one or more additional therapeutic agents, e.g., as described herein, are capable of eliciting a protective or a therapeutically effective immune response in a human against HIV-1, e.g., capable of either preventing HIV-1 infection in an uninfected individual, or in therapeutic contexts, capable of eliciting an immune response sufficient to induce immune mediated control of HIV-1 or eradicate HIV-1 in an infected individual. The immunogenicity of the fusion polypeptides can be evaluated and demonstrated, in in vitro and in vivo assays, as described herein. For example, immunogenicity of the fusion polypeptides can be demonstrated by an in vitro assay, including CD4+ and/or CD8+ T-cell activation (e.g., including cytokine expression and target killing assays) or proliferation assays. The T-cells can be activated by exposure to antigen presenting cells (APCs) (such as dendritic cells, e.g., monocyte-derived dendritic cells) that have been transfected with a polynucleotide encoding the fusion polypeptide. Such assays are known in the art and described herein. The immunogenicity of the fusion polypeptides can also be demonstrated in in vivo animal models, for example, by administering to mice, e.g., transgenic for one or more human HLA molecules (available from Jackson Laboratories or Taconic), or non-human primates, and evaluating CD4+ and/or CD8+ T-cell activation (e.g., including serum cytokine levels) or proliferation. In various embodiments, one, two, three, or more, of each polypeptide segment comprises or consists of one or more predicted T cell epitopes, e.g., as computationally or experimentally determined. In some embodiments, the fusion polypeptide comprises one or more polypeptide segments that bind to or are presented by one or more human HLA class I and/or class II alleles (e.g. 1, 2, 3, 4, 5 or 6 alleles), e.g. within a single subject or amongst multiple subjects. In some embodiments, the fusion polypeptide comprises one or more polypeptide segments that bind to or are presented by at least by a human A*0201 HLA class I molecule. In some embodiments, the fusion polypeptide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more, 8-mer, 9-mer and/or 10-mer polypeptide segments that bind to or are presented by one or more human HLA class I and/or class II alleles (e.g. 1, 2, 3, 4, 5 or 6 alleles), e.g. within a single subject. In some embodiments, the fusion polypeptide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more polypeptide segments, each 15-30 amino acids in length, that are intracellularly processed and presented by one or more human HLA class I and/or class II alleles (e.g. 1, 2, 3, 4, 5 or 6 alleles), e.g. within a single subject.

### Concatenating Polypeptide Segments

As appropriate, the one or more of the polypeptide segments can be directly abutted or fused to an adjacent segment, or can be joined, connected or linked to an adjacent segment by one or more peptide linkers. In various embodiments, the one or more peptide linkers is selected from one or more of a polyalanine linker, a polyglycine linker, a cleavable linker, a flexible linker, a rigid linker, a Nef linking sequence, and combinations thereof, e.g., within a linker or within a full-length fusion polypeptide. Illustrative fusion protein linkers that can be used in the present fusion polypeptides to connect one or more polypeptide segments are described, e.g., in Chen, et al., Adv Drug Deliv Rev. (2013) 65(10): 1357-1369. In some embodiments, the polyalanine linker comprises or consists of 2 or 3 contiguous alanine residues, e.g. AA, AAA (SEQ ID NO: 378), AAY (SEQ ID NO: 379) or AAX, wherein X is any amino acid (e.g., A, C, D, E, F, G, H, I, K, L, M, N, P, Q, R, S, T, V, Y) (SEQ ID NO: 380). In some embodiments, a polyglycine linker is used, e.g., GGS (SEQ ID NO: 419), GSG (SEQ ID NO: 420) or GGGS (SEQ ID NO:421).

In some embodiments, the cleavable linker is selected from a 2A cleavable peptide. Illustrative 2A cleavable peptides that can be used in the present fusion polypeptides to connect one or more polypeptide segments are described, e.g., in Donnelly, et al., J. Gen. Virol (2001), 82, 1027-1041 and Chng, et al., mAbs (2015) 7:2, 403-412. Illustrative cleavable peptides that can be used to link one or more polypeptide segments include without limitation 2A cleavage sequences (e.g., foot-and-mouth disease virus (F2A), equine rhinitis A virus (E2A), porcine teschovirus-1 (P2A) and Thosea asigna virus (T2A)), and furin recognition/cleavage sequences (e.g. REKR (SEQ ID NO: 382), RRKR (SEQ ID NO: 383), RAKR (SEQ ID NO: 381)). In certain embodiments, a furin recognition/cleavage sequence (e.g., REKR (SEQ ID NO: 382), RRKR (SEQ ID NO: 383), RAKR (SEQ ID NO: 381)) is combined or fused with a 2A cleavable peptide (e.g., foot-and-mouth disease virus (F2A), equine rhinitis A virus (E2A), porcine teschovirus-1 (P2A) and Thosea asigna virus (T2A)) in a single linker. See, e.g., Chng, et al., mAbs (2015) 7:2, 403-412. In various embodiments, the 2A cleavable linker comprises or consists of the amino acid sequence that is at least 90%, 91%, 92%, 93%, 94%, 95%, 96%, 97%, 98% or at least 99% identical to ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388), or comprises or consists of the amino acid sequence of ATNFSLLKQAGDVEENPGP (SEQ ID NO: 384), APVKQTLNFDLLKLAGDVESNPGP (SEQ ID NO: 385), RAKRAPVKQTLNFDLLKLAGDVESNPGP (SEQ ID NO: 386), QCTNYALLKLAGDVESNPGP (SEQ ID NO: 387), or EGRGSLLTCGDVEENPGP (SEQ ID NO: 388). As appropriate, in certain embodiments, a furin recognition/cleavage sequence can be positioned either at the N-terminus or the C-terminus of a 2A linker. In some embodiments, the cleavable linker comprises or consists of a furin recognition/cleavage site selected from the group consisting of RAKR (SEQ ID NO: 381), REKR (SEQ ID NO: 382) and RRKR (SEQ ID NO: 383). REKR (SEQ ID NO: 382) is a naturally occurring cleavable linker in HIV and SIV envelope glycoprotein precursor (Bahbouhi, et al., Biochem. J. (2002) 366, 863-872). In some embodiments, the fusion polypeptide comprises one or more Nef linking sequence comprises or consists of an amino acid sequence that is at least 95%, 96%, 97%, 98% or 99% identical to VHAGPIA (SEQ ID NO: 389), VHAGPVA (SEQ ID NO: 390), or GALDI (SEQ ID NO:391), or comprises or consists of an amino acid sequence selected from VHAGPIA (SEQ ID NO: 389), VHAGPVA (SEQ ID NO: 390) and GALDI (SEQ ID NO: 391). Illustrative linkers that can be used to link or connect one or more polypeptide segments in a fusion polypeptide are provided in Table D.

TABLE D
illustrative linkers for connecting polypeptide segments

| SEQ ID | | |
|---|---|---|
| NO: | NAME | SEQUENCE |
| | poly-alanine (2) | AA |
| 378 | poly-alanine (3) | AAA |
| 379 | poly-alanine-Tyr | AAY |
| 380 | poly-alanine-XXX | AAX (X = any amino acid) |
| 381 | furin recognition site | RAKR |
| 382 | furin recognition site | REKR |
| 383 | furin recognition site | RRKR |
| 384 | P2A | ATNFSLLKQAGDVEENPGP |
| 385 | F2A | APVKQTLNFDLLKLAGDVESNPGP |
| 386 | F2A + furin recognition site | RAKRAPVKQTLNFDLLKLAGDVESNPGP |
| 387 | E2A | QCTNYALLKLAGDVESNPGP |
| 388 | T2A | EGRGSLLTCGDVEENPGP |
| 389 | Nef natural sequence link | VHAGPIA |
| 390 | Nef natural sequence link | VHAGPVA |
| 391 | Nef natural sequence link | GALDI |
| 392 | Nef natural sequence link | GALDL |
| 419 | poly-glycine | GGS |
| 420 | poly-glycine | GSG |
| 421 | Gly3Ser | GGGS |

### Polypeptide Segments Encoded by HIV-1 Gag Gene

In various embodiments, the fusion polypeptide comprises one or more segments of one or more viral proteins, or fragments or subsequences thereof, encoded by the HIV-1 Gag gene. In some embodiments, the one or more viral proteins encoded by the HIV-1 Gag gene is selected from p17 (N-terminal matrix), p24 (capsid), p7 (nucleocapsid) and p6 (C-terminus). In some embodiments, the one or more viral proteins encoded by the HIV-1 Gag gene does not comprise any p6 components. In some embodiments, the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from: SEQ ID NOs: 68-146 and 339-342; SEQ ID NOs: 68, 69, 72, 73, 74, 75, 76, 77, 80, 81, 82, 83, 84, 85, 86, 87, 88, 89, 92, 93, 101, 102, 103, 104, 109, 110, 115, 116, 125, 126, 127, 128, 129, 130, 131, 132, 133, 134, 135, 136, 139, 140, 141, 142, 143, 144, 145 and 146; SEQ ID NOs: 76, 77, 86, 87 and 92-124; SEQ ID NOs: 76, 77, 86, 87, 94 and 95; SEQ ID NOs: 76, 86 and 94; SEQ ID NOs: 77, 87 and 95; SEQ ID NOs: 68-79 and 92-124; SEQ ID NOs: 70-71, 76-77 and 94-95; SEQ ID NOs: 78, 79, 96, 99, 100, 107, 108, 113, 114, 121, 122, 123, 124, 137 and 138; SEQ ID NOs: 78, 99, 107, 113, 121, 123 and 137; SEQ ID NOs: 78, 79, 90, 91, 97, 98, 99, 100, 105, 106, 107, 108, 111, 112, 113, 114, 117, 118, 119, 120, 121, 122, 123, 124, 137 and 138; SEQ ID NOs: 78, 90, 97, 105, 111, 117, 119 and 137; and SEQ ID NOs: 78 and 137.

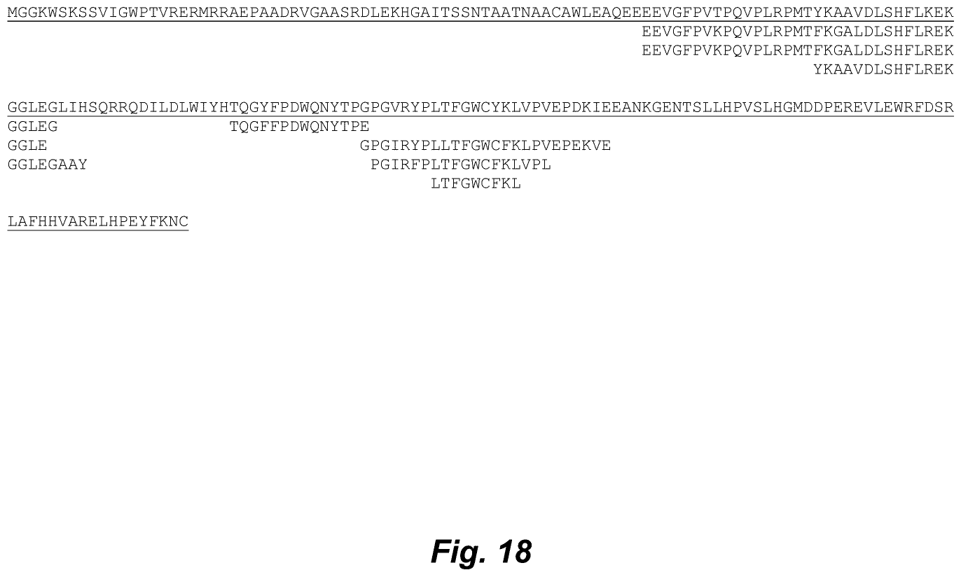

In some embodiments, the fusion polypeptide comprises at least 2 polypeptide segments, e.g., at least 3, 4, 5, 6, 7, 8, 9, 10, 11, 12, 13, 14, 15, 16, 17, 18, 19, 20, or more, segments comprising or consisting of an HIV-1 Gag amino acid sequence corresponding to amino acid residue positions selected from 31-53, 37-51, 142-166, 175-199, 183-191, 257-282, 257-290, 265-282, 288-313, 288-321, 296-313, 333-357, 337-361, 341-349, 345-353 and 429-444, wherein the amino acid positions are with respect to SEQ ID NO:404. In certain embodiments, the fusion polypeptide does not comprise 1, 2, 3, 4, 5, or more, polypeptide segments comprising or consisting of an HIV-1 Gag amino acid sequence corresponding to amino acid residue positions selected from 1-30, 54-127, 138-146, 370-428 and 445-500, or subsequences thereof, wherein the amino acid positions are with respect to SEQ ID NO:404. In some embodiments, the plurality of polypeptide segments does not, or the herein described fusion proteins do not, comprise 1, 2, 3, 4, 5, or more, polypeptide segments comprising or consisting of an HIV-1 Gag amino acid sequence of any one of SEQ ID NOs: 444-448, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 444-448, or subsequences thereof (Table K). Illustrative polypeptide segments encoded by the HIV-1 Gag gene and incorporated into the herein described fusion polypeptides (e.g., determined to be from conserved regions, predicted to bind to human HLA A*0201 and/or known to be immunogenic) are depicted as aligned to the HIV-1 HXB2 Gag reference polypeptide in FIG. 18. As used herein, numbering of a given amino acid polymer or nucleic acid polymer “corresponds to”, is “corresponding to” or is “relative to” the numbering of a selected or reference amino acid polymer or nucleic acid polymer when the position of any given polymer component (e.g., amino acid, nucleotide, also referred to generically as a “residue”) is designated by reference to the same or to an equivalent position (e.g., based on an optimal alignment or a consensus sequence) in the selected amino acid or nucleic acid polymer, rather than by the actual numerical position of the component in the given polymer.

### Polypeptide Segments Encoded by HIV-1 Nef Gene

In some embodiments, the fusion polypeptide comprises one or more segments of the viral protein encoded by the HIV-1 Nef gene. In some embodiments, the plurality of polypeptide segments comprises at least one polypeptide segment, e.g., at least 2, 3, 4, 5, 6, 7, 8, 9, 10, 11, 12, 13, 14, 15, 16, 17, 18, 19, 20, 21, 22, 23, 24, 25, or more, segments comprising or consisting of an amino acid sequence selected from: SEQ ID NOs: 147, 148, 149, 150, 151, 152, 153, 154, 155, 156, 157, 158, 159, 160, 161, 162, 163, 164, 165, 166, 167, 168, 169, 170, 171 and 172; SEQ ID NOs: 147, 148, 149, 150, 155, 156, 157, 158, 159, 160, 166, 167, 168, 169, 170 and 171; SEQ ID NOs: 149-152; SEQ ID NOs: 151-152; SEQ ID NOs: 149, 150, 151, 152, 159, 160, 161, 162, 163, 164, 166, 167, 168, 169, 170, 171, 172, 173 and 174; SEQ ID NOs: 151, 152, 161 and 162; SEQ ID NOs: 151 and 152; SEQ ID NOs: 153, 154, 172 and 173; SEQ ID NOs: 153 and 172; SEQ ID NOs: 153, 154, 155, 156, 157, 158, 163, 164, 165, 166, 167, 168, 169, 170, 171, 172 and 173; SEQ ID NOs: 153 and 165; and SEQ ID NO: 153.

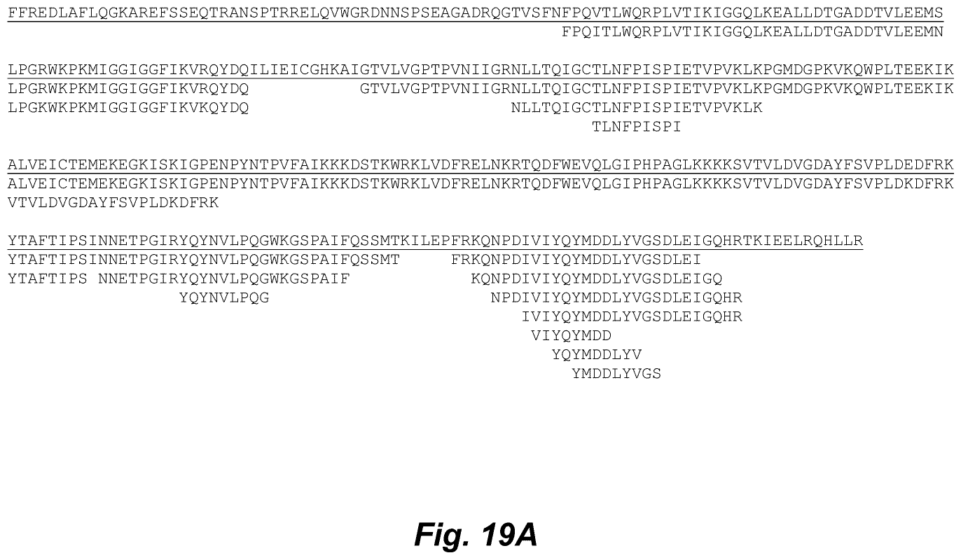

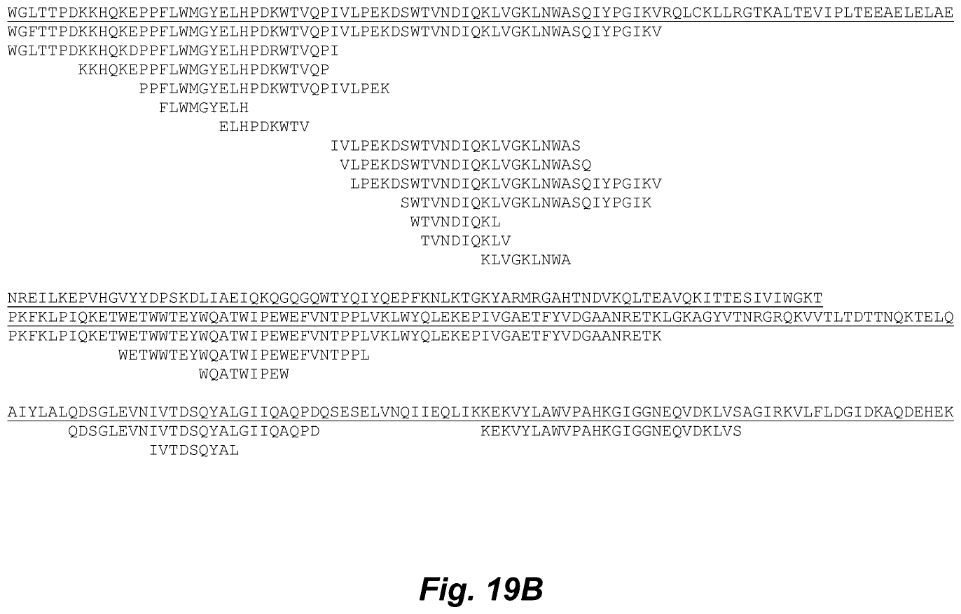

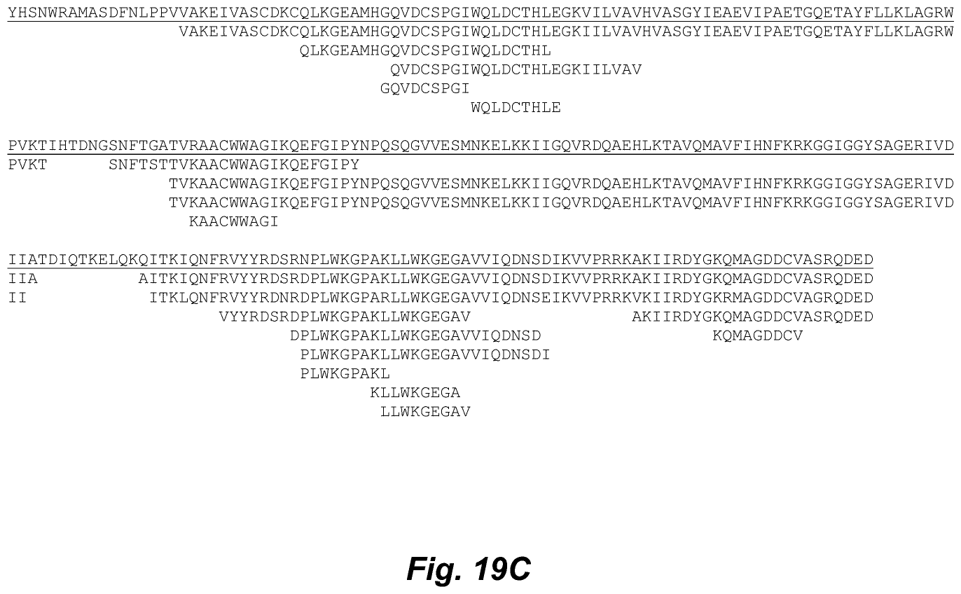

In some embodiments, the fusion polypeptide comprises at least 2 polypeptide segments, e.g., at least 3, 4, 5, 6, 7, 8, 9, 10, 11, 12, 13, 14, 15, or more, segments comprising or consisting of an HIV-1 Nef amino acid sequence corresponding to amino acid residue positions selected from 64-102, 81-102, 88-97, 91-99, 130-148, 130-154, 134-142, 134-148, 136-148, 137-145, 137-145 and 117-154, wherein the amino acid positions are with respect to SEQ ID NO:405. In certain embodiments, the fusion polypeptide does not comprise 1, 2, 3, or more, polypeptide segments comprising or consisting of an HIV-1 Nef amino acid sequence corresponding to amino acid residue positions selected from 1-63, 103-116 and 155-206, or subsequences thereof, wherein the amino acid positions are with respect to SEQ ID NO:405. In some embodiments, the plurality of polypeptide segments does not, or the herein described fusion proteins do not, comprise 1, 2, 3, or more, polypeptide segments comprising or consisting of an HIV-1 Nef amino acid sequence of any one of SEQ ID NOs: 449-451, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 449-451, or subsequences thereof (Table K). Illustrative polypeptide segments encoded by the HIV-1 Nef gene and incorporated into the herein described fusion polypeptides (e.g., determined to be from conserved regions, predicted to bind to human HLA A*0201 and/or known to be immunogenic) are depicted as aligned to the HIV-1 HXB2 Nef reference polypeptide in FIG. 19.

### Fusion Polypeptides Having Polypeptide Segments Encoded by HIV-1 Gag and Nef Genes

In some embodiments, the fusion polypeptide comprises or consists of one or more segments of viral proteins encoded by the HIV-1 Gag and Nef genes, e.g., does not comprise one or more polypeptide segments encoded by the HIV-1 Env, Pol, Tat, Rev, Vif, Vpr or Vpu genes. In some embodiments, the fusion polypeptide comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from: SEQ ID NOs: 68-79 and 92-124, 149, 150, 151, 152, 159, 160, 161, 162, 163, 164, 166, 167, 168, 169, 170, 171, 172, 173 and 174; SEQ ID NOs: 70, 71, 76, 77, 94, 95, 151, 152, 161 and 162; SEQ ID NOs: 70, 76, 94, 151 and 161; and SEQ ID NOs: 71, 77, 95, 152 and 162. Polypeptide segments included in fusion polypeptides having polypeptide segments encoded by HIV-1 Gag and Nef genes are listed in Table E.

TABLE E
polypeptide segments in GagNef fusion polypeptides (e.g., SEQ ID NOs: 353-356)

| SEQ ID | | | | |
|---|---|---|---|---|
| NO: | Gene | Start | End | SEQUENCE |
| 68 | Gag | 1 | 11 | MGARASVLSGG |
| 69 | Gag | 1 | 11 | MGARASILSGG |
| 70 | Gag | 1 | 53 | MGARASVLSGGELDRWEKIRLRPGGKKKYRLKHIVWASRELERFAVNPGLLET |
| 71 | Gag | 1 | 53 | MGARASILSGGKLDKWEKIRLRPGGRKKYKLKHIVWASRELERFAVNPGLLET |
| 72 | Gag | 13 | 25 | LDRWEKIRLRPGG |
| 73 | Gag | 13 | 25 | LDKWEKIRLRPMG |
| 74 | Gag | 19 | 27 | IRLRPGGKK |
| 75 | Gag | 19 | 27 | IRLRPGGRK |
| 76 | Gag | 31 | 53 | LKHIVWASRELERFAVNPGLLET |
| 77 | Gag | 31 | 53 | LKHLVWASRELERFALNPGLLET |
| 78 | Gag | 37 | 51 | ASRELERFAVNPGLL |
| 79 | Gag | 37 | 51 | ASRELERFALNPGLL |
| 92 | Gag | 147 | 217 | ISPRTLNAWVKVVEEKAFSPEVIPMFSALSEGATPQDLNTMLNTVGGHQAAMQMLKETINEEAAEWDRLHP |
| 93 | Gag | 147 | 217 | LSPRTLNAWVKVIEEKAFSPEVIPMFTALSEGATPHDLNTMLNTIGGHQAAMQMLKDTINEEAAEWDRVHP |
| 94 | Gag | 147 | 369 | ISPRTLNAWVKVVEEKAFSPEVIPMFSALSEGATPQDLNTMLNTVGGHQAAMQMLKETINEEAAEWDRLHPVHA |
| | | | | GPIAPGQMREPRGSDIAGTTSTLQEQIGWMTNNPPIPVGEIYKRWIILGLNKIVRMYSPTSILDIRQGPKEPFR |
| | | | | DYVDRFYKTLRAEQASQEVKNWMTETLLVQNANPDCKTILKALGPAATLEEMMTACQGVGGPGHKARVLAEAMS |
| | | | | Q |
| 95 | Gag | 147 | 369 | LSPRILNAWVKVIEEKAFSPEVIPMFTALSEGATPHDLNTMLNTIGGHQAAMQMLKDTINEEAAEWDRVHPVHA |
| | | | | GPVAPGQMRDPRGSDIAGSTSTLQEQIAWMTNNPPIPVGDIYKRWIIMGLNKIVRMYSPVSILDIKQGPKEPFR |
| | | | | DYVDRFYRTLRAEQASQDVKNWMTETLLVQNSNPDCKTILKALGPGATLEEMMSACQGVGGPSHKARVLAEAMC |
| | | | | Q |
| 96 | Gag | 150 | 158 | RTLNAWVKV |
| 97 | Gag | 175 | 199 | LSEGATPQDLNTMLNTVGGHQAAMQ |
| 98 | Gag | 175 | 199 | LSEGATPHDLNTMLNTIGGHQAAMQ |
| 99 | Gag | 183 | 191 | DLNTMLNTV |
| 100 | Gag | 183 | 191 | DLNTMLNTI |
| 101 | Gag | 225 | 251 | PGQMREPRGSDIAGTTSTLQEQIGWMT |
| 102 | Gag | 225 | 251 | PGQMRDPRGSDIAGSTSTLQEQIAWMT |
| 103 | Gag | 253 | 285 | NPPIPVGEIYKRWIILGLNKIVRMYSPTSILDI |
| 104 | Gag | 253 | 285 | NPPIPVGDIYKRWIIMGLNKIVRMYSPVSILDI |
| 339 | Gag | 257 | 282 | PVGEIYKRWIILGLNKIVRMYSPTSI |
| 340 | Gag | 257 | 282 | PVGDIYKRWIIMGLNKIVRMYSPVSI |
| 105 | Gag | 257 | 290 | PVGEIYKRWIILGLNKIVRMYSPTSILDIRQGPK |
| 106 | Gag | 257 | 290 | PVGDIYKRWIIMGLNKIVRMYSPVSILDIKQGPK |
| 107 | Gag | 265 | 282 | WIILGLNKIVRMYSPTSI |
| 108 | Gag | 265 | 282 | WIIMGLNKIVRMYSPVSI |
| 109 | Gag | 281 | 314 | SILDIRQGPKEPFRDYVDRFYKTLRAEQASQEVK |
| 110 | Gag | 281 | 314 | SILDIKQGPKEPFRDYVDRFYRTLRAEQASQDVK |
| 341 | Gag | 288 | 313 | GPKEPFRDYVDRFYKTLRAEQASQEV |
| 342 | Gag | 288 | 313 | GPKEPFRDYVDRFYRTLRAEQASQDV |
| 111 | Gag | 288 | 321 | GPKEPFRDYVDRFYKTLRAEQASQEVKNWMTETL |
| 112 | Gag | 288 | 321 | GPKEPFRDYVDRFYRTLRAEQASQDVKNWMTETL |
| 113 | Gag | 296 | 313 | YVDRFYKTLRAEQASQEV |
| 114 | Gag | 296 | 313 | YVDRFYRTLRAEQASQDV |
| 115 | Gag | 311 | 369 | QEVKNWMTETLLVQNANPDCKTILKALGPAATLEEMMTACQGVGGPGHKARVLAEAMSQ |
| 116 | Gag | 311 | 369 | QDVKNWMTETLLVQNSNPDCKTILKALGPGATLEEMMSACQGVGGPSHKARVLAEAMCQ |
| 117 | Gag | 333 | 357 | ILKALGPAATLEEMMTACQGVGGPG |
| 118 | Gag | 333 | 357 | ILKALGPGATLEEMMSACQGVGGPS |
| 119 | Gag | 337 | 361 | LGPAATLEEMMTACQGVGGPGHKAR |
| 120 | Gag | 337 | 361 | LGPGATLEEMMSACQGVGGPSHKAR |
| 121 | Gag | 341 | 349 | ATLEEMMTA |
| 122 | Gag | 341 | 349 | ATLEEMMSA |
| 123 | Gag | 345 | 353 | EMMTACQGV |
| 124 | Gag | 345 | 353 | EMMSACQGV |
| 149 | Nef | 64 | 82 | EEVGFPVRPQVPLRPMTYK |
| 150 | Nef | 64 | 82 | EEVGFPVKPQVPLRPMTFK |
| 151 | Nef | 64 | 99 | EEVGFPVKPQVPLRPMTFKGALDLSHFLREKGGLEG |
| 152 | Nef | 64 | 99 | EEVGFPVRPQVPLRPMTYKGALDLSHFLKEKGGLEG |
| 159 | Nef | 117 | 132 | TQGYFPDWQNYTPGPG |
| 160 | Nef | 117 | 132 | TQGFFPDWQNYTPEPG |
| 161 | Nef | 117 | 148 | TQGFFPDWQNYTPEPGIRFPLTFGWCFKLVPL |
| 162 | Nef | 117 | 148 | TQGYFPDWQNYTPGPGTRYPLTFGWCFKLVPV |
| 163 | Nef | 130 | 148 | EPGIRFPLTFGWCFKLVPL |
| 164 | Nef | 130 | 148 | GPGTRYPLTFGWCFKLVPV |
| 166 | Nef | 134 | 142 | RYPLTFGWC |
| 167 | Nef | 134 | 142 | RFPLTFGWC |
| 168 | Nef | 134 | 148 | RYPLTFGWCFKLVPV |
| 169 | Nef | 134 | 148 | RFPLTFGWCFKLVPL |
| 170 | Nef | 136 | 148 | PLTFGWCFKLVPV |
| 171 | Nef | 136 | 148 | PLCFGWCFKLVPL |
| 172 | Nef | 137 | 145 | LTFGWCFKL |
| 173 | Nef | 137 | 145 | LCFGWCFKL |

In some embodiments, the fusion polypeptide comprises or consists of the following polypeptide segments in sequential order, from N-terminus to C-terminus, optionally joined or connected by one or more linkers: SEQ ID NOs: 70, 76, 94, 151 and 161; or SEQ ID NOs: 71, 77, 95, 152 and 162.

In some embodiments, the fusion polypeptide comprises or consists of an amino acid sequence of any one of SEQ ID NOs: 351-356 and 430, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 351-356 and 430.

Modifications may be made in the structure of the fusion polypeptides and polynucleotides encoding such fusion polypeptides, described herein, and still obtain a functional molecule that encodes a variant or derivative polypeptide with desirable (e.g., immunogenic) characteristics. When it is desired to alter the amino acid sequence of a polypeptide to create an equivalent, or even an improved, variant or portion of a fusion polypeptide described herein, one skilled in the art will typically change one or more of the codons of the encoding DNA sequence.

For example, certain amino acids may be substituted for other amino acids in a protein structure without appreciable loss of its ability to bind other polypeptides (e.g., antigens) or cells. Since it is the binding capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the polypeptide sequences of the disclosed fusion polypeptides, or corresponding DNA sequences that encode such fusion polypeptides without appreciable loss of their biological utility or activity.

In many instances, a polypeptide variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J. (1990) Unified Approach to Alignment and Phylogenes pp. 626-645 Methods in Enzymology vol. 183, Academic Press, Inc., San Diego, CA; Higgins, D. G. and Sharp, P. M. (1989) CABIOS 5: 151-153; Myers, E. W. and Muller W. (1988) CABIOS 4:11-17; Robinson, E. D. (1971) Comb. Theor 77: 105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, CA; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides described herein. Software for performing BLAST analyses is publicly available through the National Center for Biotechnology Information (blast.ncbi.nlm.nih.gov/Blast.cgi).

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a word length (W) of 11, and expectation (E) of 10, and the BLOSUM62 scoring matrix (see Henikoff and Henikoff (1989) Proc. Natl. Acad. Sci. USA 89: 10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

A “polypeptide variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences described herein and evaluating one or more biological activities of the polypeptide as described herein and/or using any of a number of techniques well known in the art. The term “variant” may also refer to any naturally occurring or engineered molecule comprising one or more nucleotide or amino acid mutations.

### Polypeptide Segments Encoded by HIV-1 Env Gene

In some embodiments, the fusion polypeptides comprise one or more segments of one or more viral proteins encoded by the HIV-1 Env gene. In certain embodiments, the one or more viral proteins encoded by the HIV-1 Env gene is selected from gp120 and gp41.

In various embodiments, the fusion polypeptide comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from: SEQ ID NOs: 1-67 and 338; SEQ ID NOs: 2, 3, 8, 9, 13, 14, 17, 18, 23, 24, 25, 26, 28, 29, 31, 32, 33, 34, 35, 36, 37, 38, 39, 40, 43, 44, 45, 46, 47, 48, 49, 50, 51, 52, 53, 54, 55, 56, 58, 59, 62, 63, 64, 65, 66 and 67; SEQ ID NOs: 4, 5, 6, 7, 11, 12, 13, 14, 15, 16, 28, 29, 30, 37, 38, 41, 42, 43, 44, 45, 46, 47, 48, 49, 50, 51, 52, 53, 54, 55, 56, 57, 58, 59, 60, 61 and 338; SEQ ID NOs: 4, 5, 6, 7, 11, 12, 13, 14, 15, 16, 28, 29, 30, 37, 38, 41 and 42; SEQ ID NOs: 28, 29, 30 and 41-56; SEQ ID NOs: 28, 29, 41 and 42; SEQ ID NOs: 4, 5, 6, 7, 11, 12, 13, 14, 15, 16, 37 and 38; SEQ ID NOs: 4, 5, 11, 12, 37 and 38; SEQ ID NOs: 6, 7, 15, 16, 21, 22, 30, 60 and 61; SEQ ID NOs: 6, 15, 21, 30 and 60; SEQ ID NOs: 1, 2, 3, 4, 5, 6, 7, 10, 11, 12, 13, 14, 15, 16, 19, 20, 27, 55, 56, 57, 58, 59, 60, 61 and 338; SEQ ID NOs: 1, 10, 19, 27, 55, 56 and 57; and SEQ ID NOs: 6, 15 and 60.

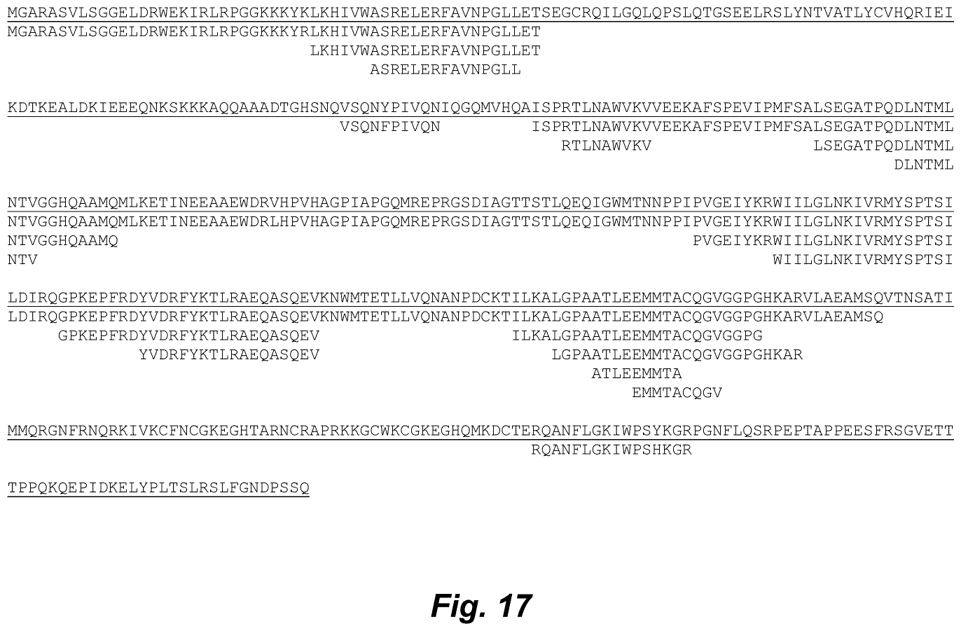

In various embodiments, the fusion polypeptide comprises at least 2 polypeptide segments, e.g., at least 3, 4, 5, 6, 7, 8, 9, 10, 11, 12, 13, 14, or more, segments comprising or consisting of an HIV-1 Env amino acid sequence corresponding to amino acid residue positions selected from 28-52, 34-48, 34-47, 36-44, 59-83, 64-83, 66-83, 67-75, 113-137, 235-259, 586-594, 586-610, 589-606 and 594-602, wherein the amino acid positions are with respect to SEQ ID NO:403. In certain embodiments, the fusion polypeptide does not comprise 1, 2, 3, 4, 5, 6, or more, polypeptide segments comprising or consisting of an HIV-1 Env amino acid sequence corresponding to amino acid residue positions selected from 1-27, 53-58, 84-112, 138-234, 269-474, 490-501, 611-856, or subsequences thereof, wherein the amino acid positions are with respect to SEQ ID NO:403. In some embodiments, the plurality of polypeptide segments does not, or the herein described fusion proteins do not, comprise 1, 2, 3, 4, 5, or more, polypeptide segments comprising or consisting of an HIV-1 Env amino acid sequence of any one of SEQ ID NOs: 437-443, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 437-443, or subsequences thereof (Table K). Illustrative polypeptide segments encoded by the HIV-1 Env gene and incorporated into the herein described fusion polypeptides (e.g., determined to be from conserved regions, predicted to bind to human HLA A*0201 and/or known to be immunogenic) are depicted as aligned to the HIV-1 HXB2 Env reference polypeptide in FIG. 17.

### Polypeptide Segments Encoded by HIV-1 Pol Gene

In some embodiments, the fusion polypeptides comprise of one or more segments of one or more viral proteins encoded by the HIV-1 Pol gene. In various embodiments, the one or more viral proteins encoded by the HIV-1 Pol gene is selected from one or more of protease (PR), reverse transcriptase (RT), and integrase (INT).

In some embodiments, the plurality of polypeptide segments comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from: SEQ ID NOs: 174-337 and 343-344; SEQ ID NOs: 174, 175, 178, 179, 180, 181, 182, 183, 184, 185, 193, 194, 195, 196, 197, 198, 199, 200, 203, 204, 205, 206, 207, 208, 213, 214, 221, 222, 236, 237, 245, 246, 247, 248, 249, 250, 251, 252, 253, 254, 255, 256, 257, 258, 263, 264, 266, 267, 268, 269, 270, 271, 272, 273, 278, 279, 280, 281, 282, 283, 284, 285, 286, 287, 288, 289, 290, 291, 292, 293, 294, 295, 305, 306, 307, 308, 309, 310, 313, 314, 315, 316, 317, 318, 321, and 322; SEQ ID NOs: 180, 181, 182, 183, 184, 185, 186, 187, 190, 191, 192, 193, 194, 195, 196, 221, 222, 294, 295, 296, 297, 298, 299, 300, 301, 305, 306, 307, 308, 311, 312, 321, 322, 323, 324, 325, 326, 327, 328, 329, 330, 331, 332, 333, 334, 335, 336 and 337; SEQ ID NOs: 180, 181, 186, 187, 221, 222, 294, 295, 307, 308, 321 and 322; SEQ ID NOs: 176, 177, 178, 179, 180, 181, 182, 183, 184, 185, 186, 187, 188, 189, 190, 191, 192, 193, 194, 195, 196, 197, 198, 199, 200, 201, 202, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 257, 258, 259, 260, 261, 262, 263, 264, 265, 266, 267, 282, 283, 294, 295, 296, 297, 298, 299, 300, 301, 302, 305, 306, 319, 320, 321, 322, 323, 324, 325, 326, 327, 328, 329, 330, 331, 332, 333, 334, 335, 336 and 337; SEQ ID NOs: 176, 177, 188, 189, 213, 214, 223, 224, 259, 260, 282, 283, 294, 295, 305, 306, 319, and 320; SEQ ID NOs: 180, 181, 186, 187, 221, 222, 294, 295, 321 and 322; SEQ ID NOs: 182-202, 292-302, 305, 306; SEQ ID NOs: 188, 189, 294, 295, 305, 306; SEQ ID NOs: 176, 177, 178, 179, 180, 181, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 257, 258, 259, 260, 261, 262, 263, 264, 265, 266, 267, 282, 283, 319, 320, 321, 322, 323, 324, 325, 326, 327, 328, 329, 330, 331, 332, 333, 334, 335, 336 and 337; SEQ ID NOs: 176, 177, 213, 214, 223, 224, 259, 260, 282, 283, 319 and 320; SEQ ID NOs: 192, 201, 202, 215, 216, 217, 218, 219, 220, 229, 230, 231, 240, 241, 242, 243, 244, 265, 276, 277, 298, 299, 302, 311, 312, 327, 328, 331, 332, 333, 336 and 337; SEQ ID NOs: 192, 201, 215, 217, 219, 229, 230, 240, 241, 243, 265, 276, 298, 302, 311, 327, 331, 333 and 336; SEQ ID NOs: 190, 191, 192, 197, 198, 209, 210, 211, 212, 213, 214, 215, 216, 217, 218, 219, 220, 225, 226, 227, 228, 229, 230, 231, 232, 233, 234, 235, 238, 239, 261, 262, 274, 275, 276, 277, 296, 297, 298, 299, 300, 301, 302, 303, 304, 323, 324, 325, 326, 327, 328, 329, 330, 331, 332, 333, 334, 335, 336, 337, 343, 344, 375 and 376; SEQ ID NOs: 190, 197, 209, 210, 211, 225, 227, 234, 238, 261, 296, 300, 303, 323, 325, 329, 334; and SEQ ID NOs: 192, 215, 217, 219, 229, 230, 276, 298, 302, 327, 331, 333 and 336.

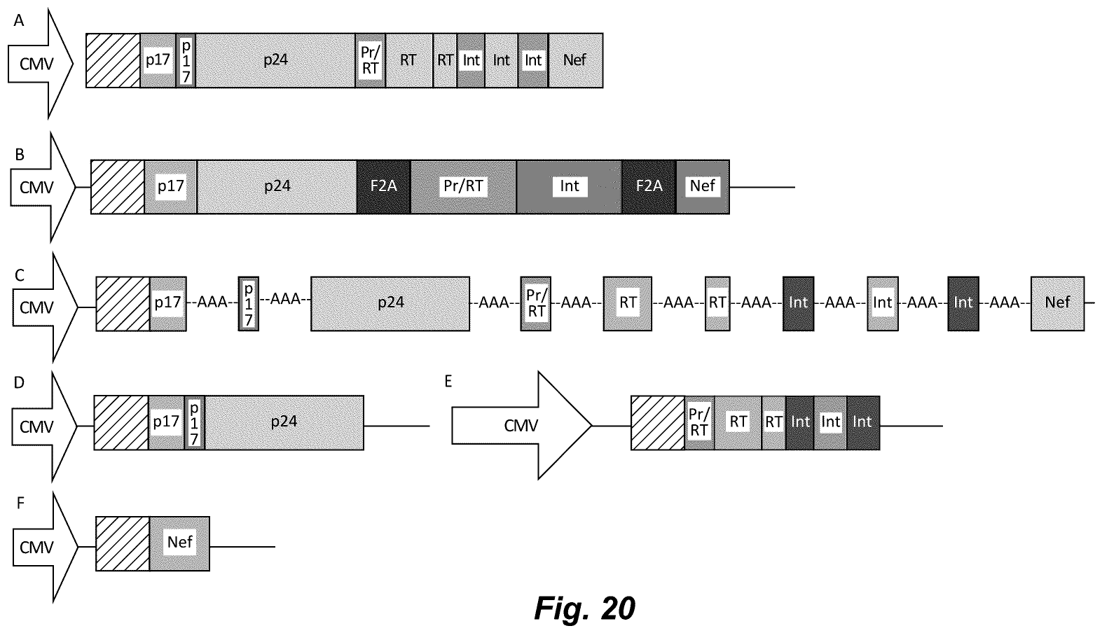

In some embodiments, the fusion polypeptide comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 39, 30, or more, segments comprising or consisting of an HIV-1 Pol amino acid sequence corresponding to amino acid residue positions selected from 144-168, 152-160, 291-315, 326-350, 328-352, 330-354, 333-354, 334-342, 336-344, 338-346, 374-398, 380-404, 382-390, 388-396, 399-423, 400-424, 406-430, 553-577, 642-666, 650-658, 759-783, 767-775, 768-792, 776-784, 834-858, 940-964, 947-971, 948-956, 948-972, 955-963, 956-964, 980-1003 and 988-996, wherein the amino acid positions are with respect to SEQ ID NO:406. In certain embodiments, the fusion polypeptide does not comprise 1, 2, 3, 4, 5, 6, 7, 8, 9, 10, or more, polypeptide segments comprising or consisting of an HIV-1 Pol amino acid sequence corresponding to amino acid residue positions selected from 1-55, 118-128, 321-325, 355-366, 432-541, 607-641, 667-682, 709-746, 828-833, 921-930, or subsequences thereof, wherein the amino acid positions are with respect to SEQ ID NO:406. In some embodiments, the plurality of polypeptide segments does not, or the herein described fusion proteins do not, comprise 1, 2, 3, 4, 5, or more, polypeptide segments comprising or consisting of an HIV-1 Pol amino acid sequence of any one of SEQ ID NOs: 452-461, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 452-461, or subsequences thereof (Table K). Illustrative polypeptide segments encoded by the HIV-1 Pol gene and incorporated into the herein described fusion polypeptides (e.g., determined to be from conserved regions, predicted to bind to human HLA A*0201 and/or known to be immunogenic) are depicted as aligned to the HIV-1 HXB2 Pol reference polypeptide in FIGS. 20A-C.

In some embodiments, a fusion polypeptide comprising polypeptide segments encoded by the HIV-1 Pol gene does not comprise the amino acid sequence or motif YMDD (SEQ ID NO: 462) or YVDD (SEQ ID NO: 463). In some embodiments, the fusion polypeptide does not comprise one or more amino acid sequences selected from SEQ ID NOs: 215, 216, 217, 218, 219 and 220. In some embodiments, the fusion polypeptide does not comprise one or more amino acid sequences selected from SEQ ID NOs: 209, 210, 211, 212, 213, 214, 343 and 344.

### Fusion Polypeptides Having Polypeptide Segments Encoded by HIV-1 Env and Pol Genes

In some embodiments, the fusion polypeptide comprises or consists of one or more segments of viral proteins encoded by the HIV-1 Env and Pol genes, e.g., does not comprise one or more polypeptide segments encoded by the HIV-1 Gag, Nef, Tat, Rev, Vif, Vpr or Vpu genes.

In some embodiments, the fusion polypeptide comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from: SEQ ID NOs: 4, 5, 6, 7, 11, 12, 13, 14, 15, 16, 28, 29, 30, 37, 38, 41, 42, 43, 44, 45, 46, 47, 48, 49, 50, 51, 52, 53, 54, 55, 56, 57, 58, 59, 60, 61, 176, 177, 178, 179, 180, 181, 182, 183, 184, 185, 186, 187, 188, 189, 190, 191, 192, 193, 194, 195, 196, 197, 198, 199, 200, 201, 202, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 257, 258, 259, 260, 261, 262, 263, 264, 265, 266, 267, 282, 283, 294, 295, 296, 297, 298, 299, 300, 301, 302, 305, 306, 319, 320, 321, 322, 323, 324, 325, 326, 327, 328, 329, 330, 331, 332, 333, 334, 335, 336, 337 and 338; SEQ ID NOs: 4, 5, 6, 7, 11, 12, 13, 14, 15, 16, 28, 29, 30, 37, 38, 41, 42, 176, 177, 188, 189, 213, 214, 223, 224, 259, 260, 282, 283, 294, 295, 305, 306, 319 and 320; SEQ ID NOs: 28, 29, 30, 41-56, 182-202, 292-302, 305 and 306; SEQ ID NOs: 28, 29, 41, 42, 188, 189, 294, 295, 305 and 306; SEQ ID NOs: 4, 5, 6, 7, 11, 12, 13, 14, 15, 16, 37, 38, 176, 177, 178, 179, 180, 181, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 257, 258, 259, 260, 261, 262, 263, 264, 265, 266, 267, 282, 283, 319, 320, 321, 322, 323, 324, 325, 326, 327, 328, 329, 330, 331, 332, 333, 334, 335, 336 and 337; and SEQ ID NOs: 4, 5, 11, 12, 37, 38, 176, 177, 213, 214, 223, 224, 259, 260, 282, 283, 319 and 320. Polypeptide segments included in fusion polypeptides having polypeptide segments encoded by HIV-1 Env and Pol genes are listed in Table F.

In some embodiments, the fusion polypeptide comprises the following polypeptide segments in sequential order, from N-terminus to C-terminus, optionally joined or connected by one or more linkers: SEQ ID NOs: 188, 305, 28, 41, 294, 4, 176, 11, 319, 259, 282, 223, 213 and 37; SEQ ID NOs: 188, 305, 28, 41 and 294; SEQ ID NOs: 4, 176, 11, 319, 259, 282, 223, 213 and 37; SEQ ID NOs: 189, 306, 29, 42, 295, 5, 177, 12, 320, 260, 283, 224, 214 and 38; SEQ ID NOs: 189, 306, 29, 42 and 295; SEQ ID NOs: 5, 177, 12, 320, 260, 283, 224, 214 and 38; SEQ ID NOs: 305, 319, 259, 282, 223, 213, 294, 176 and 188; SEQ ID NOs: 306, 320, 260, 283, 224, 214, 295, 177 and 189; SEQ ID NOs: 305, 294, 223, 213, 176, 259, 319, 188 and 282; SEQ ID NOs: 306, 295, 224, 214, 177, 260, 320, 189 and 283; SEQ ID NOs: 305, 294, 319, 259, 282, 223, 176, and 188; SEQ ID NOs: 306, 295, 320, 260, 283, 224, 177 and 189; SEQ ID NOs: 305, 223, 294, 176, 259, 319, 188 and 282; or SEQ ID NOs: 306, 224, 295, 177, 260, 320, 189 and 283.

In some embodiments, the fusion polypeptide comprises or consists of an amino acid sequence of any one of SEQ ID NOs: 357-366 and 407-410, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 357-366 and 407-410.

### Fusion Polypeptides Having Polypeptide Segments Encoded by HIV-1 Gag, Nef and Pol Genes

In some embodiments, the fusion polypeptide comprises or consists of one or more segments of viral proteins encoded by the HIV-1 Gag, Nef and Pol genes, e.g., does not comprise one or more polypeptide segments encoded by the HIV-1 Env, Tat, Rev, Vif, Vpr or Vpu genes.

In some embodiments, the fusion polypeptide comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from: SEQ ID NOs: 76, 77, 86, 87, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 149, 150, 151, 152, 180, 181, 182, 183, 184, 185, 186, 187, 190, 191, 192, 193, 194, 195, 196, 221, 222, 294, 295, 296, 297, 298, 299, 300, 301, 305, 306, 307, 308, 311, 312, 321, 322, 323, 324, 325, 326, 327, 328, 329, 330, 331, 332, 333, 334, 335, 336, 337, 339, 340, 341 and 342; and SEQ ID NOs: 76, 77, 86, 87, 94, 95, 151, 152, 181, 182, 186, 187, 221, 222, 294, 195, 307, 308, 321, 322. Polypeptide segments included in fusion polypeptides having polypeptide segments encoded by HIV-1 Gag, Nef and Pol genes are listed in Table G.

In some embodiments, the fusion polypeptide comprises the following polypeptide segments in sequential order, from N-terminus to C-terminus, optionally joined or connected by one or more linkers: SEQ ID NOs: 76, 86, 94, 180, 186, 221, 294, 307, 321 and 151; or SEQ ID NOs: 77, 87, 95, 181, 187, 222, 295, 308, 322 and 152.

In some embodiments, the fusion polypeptide comprises or consists of an amino acid sequence of any one of SEQ ID NOs: 345-350, the sequences in Table 1, and SEQ ID NOs: 422-424, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NO: 345-350, the sequences in Table 1, and SEQ ID NOs: 422-424.

TABLE F
polypeptide segments in Pol/PolEnv fusion polypeptides (e.g., SEQ ID NOs: 357-366, 407-410))

| SEQ ID | | | | |
|---|---|---|---|---|
| NO: | Gene | Start | End | Sequence |
| 4 | Env | 34 | 47 | LWVTVYYGVPVWKE |
| 5 | Env | 34 | 47 | LWVTIYYGVPVWKD |
| 6 | Env | 36 | 44 | VTVYYGVPV |
| 7 | Env | 36 | 44 | VTIYYGVPV |
| 11 | Env | 65 | 83 | AHNVWATHACVPTDPNPQE |
| 12 | Env | 65 | 83 | VHNIWATHACVPTDPSPQE |
| 13 | Env | 66 | 83 | HNVWATHACVPTDPNPQE |
| 14 | Env | 66 | 83 | HNIWATHACVPTDPSPQE |
| 15 | Env | 67 | 75 | NVWATHACV |
| 16 | Env | 67 | 75 | NIWATHACV |
| 28 | Env | 241 | 268 | NVSTVQCTHGIRPVVSTQLLLNGSLAEE |
| 29 | Env | 241 | 268 | NISTVQCTHGIKPVVSTQLLLNGSLAEK |
| 30 | Env | 243 | 251 | STVQCTHGI |
| 37 | Env | 475 | 489 | MRDNWRSELYKYKVV |
| 38 | Env | 475 | 489 | MKDNWRSELYRYKVV |
| 41 | Env | 502 | 606 | KRRVVQREKRAVGIGAMFLGFLGAAGSTMGAASITLTVQARQLLSGIVQQQNNLLRAIEAQQHLLQLTVWGIKQL |
| | | | | QARVLAVERYLKDQQLLGIWGCSGKLICTT |
| 42 | Env | 502 | 606 | RRRVVQREKRAIGLGAVFLGFLGTAGSTMGAASMTLTVQARLLLSGIVQQQSNLLRAIEAQQHMLQLTVWGIKQL |
| | | | | QARILAVERYLRDQQLLGIWGCSGRLICTT |
| 43 | Env | 519 | 534 | FLGFLGAAGSTMGAAS |
| 44 | Env | 519 | 534 | FLGFLGTAGSTMGAAA |
| 45 | Env | 533 | 606 | ASITLTVQARQLLSGIVQQQNNLLRAIEAQQHLLQLTVWGIKQLQARVLAVERYLKDQQLLGIWGCSGKLICTT |
| 46 | Env | 533 | 606 | ASMTLTVQARLLLSGIVQQQSNLLRAIEAQQHMLQLTVWGIKQLQARILAVERYLRDQQLLGIWGCSGRLICTT |
| 47 | Env | 536 | 556 | TLTVQARQLLSGIVQQQNNLL |
| 48 | Env | 536 | 556 | TLTVQARLLLSGIVQQQSNLL |
| 49 | Env | 554 | 564 | NLLRAIEAQQH |
| 50 | Env | 554 | 564 | NLLKAIEAQQH |
| 51 | Env | 558 | 584 | AIEAQQHLLQLTVWGIKQLQARVLAVE |
| 52 | Env | 558 | 584 | AIEAQQHMLQLTVWGIKQLQARILAVE |
| 53 | Env | 584 | 592 | ERYLKDQQL |
| 54 | Env | 584 | 592 | ERYLRDQQL |
| 55 | Env | 586 | 594 | YLKDQQLLG |
| 56 | Env | 586 | 594 | YLRDQQLLG |
| 57 | Env | 586 | 610 | YLKDQQLLGIWGCSGKLICTTAVPW |
| 338 | Env | 586 | 610 | YLRDQQLLGLWGCSGKLICPTAVPW |
| 58 | Env | 589 | 606 | DQQLLGIWGCSGKLICTT |
| 59 | Env | 589 | 606 | DQQLLGLWGCSGKLICPT |
| 60 | Env | 594 | 602 | GIWGCSGKL |
| 61 | Env | 594 | 602 | GLWGCSGKL |
| 176 | Pol | 56 | 117 | FPQITLWQRPLVTIKIGGQLKEALLDTGADDTVLEEMNLPGRWKPKMIGGIGGFIKVRQYDQ |
| 177 | Pol | 56 | 117 | LPQITLWQRPIVTIKIGGQIKEALLDTGADDTVLEDMNLPGKWKPKMIGGIGGFIKVKQYDQ |
| 178 | Pol | 72 | 91 | GGQLKEALLDTGADDTVLEE |
| 179 | Pol | 72 | 91 | GGQIKEALLDTGADDTVLED |
| 180 | Pol | 94 | 117 | LPGRWKPKMIGGIGGFIKVRQYDQ |
| 181 | Pol | 94 | 117 | LPGKWKPKMIGGIGGFIKVKQYDQ |
| 182 | Pol | 129 | 260 | GTVLVGPTPVNIIGRNLLTQIGCTLNFPISPIETVPVKLKPGMDGPKVKQWPLTEEKIKALVEICTEMEKEGKIS |
| | | | | KIGPENPYNTPVFAIKKKDSTKWRKLVDFRELNKRIQDFWEVQLGIPHPAGLKKKKS |
| 183 | Pol | 129 | 260 | GTVLIGPTPVNIIGRNLLTQLGCTLNFPISPIDTVPVKLKPGMDGPRVKQWPLTEEKIKALIEICTEMEKEGKIS |
| | | | | RIGPENPYNTPIFAIKKKDGTKWRKLVDFRELNKKTQDFWEVQLGIPHPSGLKKKKS |
| 184 | Pol | 129 | 277 | GTVLVGPTPVNIIGRNLLTQIGCTLNFPISPIETVPVKLKPGMDGPKVKQWPLTEEKIKALVEICTEMEKEGKIS |
| | | | | KIGPENPYNTPVFAIKKKDSTKWRKLVDFRELNKRTQDFWEVQLGIPHPAGLKKKKSVTVLDVGDAYFSVPLDK |
| 185 | Pol | 129 | 277 | GTVLIGPTPVNIIGRNLLTQLGCTLNFPISPIDTVPVKLKPGMDGPRVKQWPLTEEKIKALIEICTEMEKEGKIS |
| | | | | RIGPENPYNTPIFAIKKKDGTKWRKLVDFRELNKKTQDFWEVQLGIPHPSGLKKKKSVTILDVGDAYFSIPLDK |
| 186 | Pol | 129 | 289 | GTVLVGPTPVNIIGRNLLTQIGCTLNFPISPIETVPVKLKPGMDGPKVKQWPLTEEKIKALVEICTEMEKEGKIS |
| | | | | KIGPENPYNTPVFAIKKKDSTKWRKLVDFRELNKRTQDFWEVQLGIPHPAGLKKKKSVTVLDVGDAYFSVPLDKD |
| | | | | FRKYTAFTIPS |
| 187 | Pol | 129 | 289 | GTVLIGPTPVNIIGRNLLTQLGCTLNFPISPIDTVPVKLKPGMDGPRVKQWPLTEEKIKALIEICTEMEKEGKIS |
| | | | | RIGPENPYNTPIFAIKKKDGTKWRKLVDFRELNKKTQDFWEVQLGIPHPSGLKKKKSVTVLDIGDAYFSVPLDKE |
| | | | | FRKYTAFTVPS |
| 188 | Pol | 129 | 320 | GTVLVGPTPVNIIGRNLLTQIGCTLNFPISPIETVPVKLKPGMDGPKVKQWPLTEEKIKALVEICTEMEKEGKIS |
| | | | | KIGPENPYNTPVFAIKKKDSTKWRKLVDFRELNKRIQDFWEVQLGIPHPAGLKKKKSVTVLDVGDAYFSVPLDKD |
| | | | | FRKYTAFTIPSINNETPGIRYQYNVLPQGWKGSPAIFQSSMT |
| 189 | Pol | 129 | 320 | GTVLIGPTPVNIIGRNLLTQLGCTLNFPISPIDTVPVKLKPGMDGPRVKQWPLTEEKIKALIEICTEMEKEGKIS |
| | | | | RIGPENPYNTPIFAIKKKDGTKWRKLVDFRELNKKTQDFWEVQLGIPHPSGLKKKKSVTVLDIGDAYFSVPLDKE |
| | | | | FRKYTAFTVPSTNNETPGVRYQYNVLPMGWKGSPAIFQCSMT |
| 190 | Pol | 144 | 168 | NLLTQIGCTLNFPISPIETVPVKLK |
| 191 | Pol | 144 | 168 | NLLTQLGCTLNFPISPIDTVPVKLK |
| 192 | Pol | 152 | 160 | TLNFPISPI |
| 193 | Pol | 254 | 277 | GLKKKKSVTVLDVGDAYFSVPLDK |
| 194 | Pol | 254 | 277 | GLKKNKSVTVLDVGDAYFSIPLDK |
| 195 | Pol | 278 | 289 | DFRKYTAFTIPS |
| 196 | Pol | 278 | 289 | EFRKYTAFTVPS |
| 197 | Pol | 291 | 315 | NNETPGIRYQYNVLPQGWKGSPAIF |
| 198 | Pol | 291 | 315 | NNETPGVRYQYNVLPMGWKGSPAIF |
| 199 | Pol | 291 | 320 | NNETPGIRYQYNVLPQGWKGSPAIFQSSMT |
| 200 | Pol | 291 | 320 | NNETPGVRYQYNVLPMGWKGSPAIFQCSMT |
| 201 | Pol | 299 | 307 | YQYNVLPQG |
| 202 | Pol | 299 | 307 | YQYNVLPMG |
| 213 | Pol | 333 | 354 | IVIYQYMDDLYVGSDLEIGQHR |
| 214 | Pol | 333 | 354 | IVIYQYVDDLYVGSDLEIEQHR |
| 215 | Pol | 334 | 342 | VIYQYMDDL |
| 216 | Pol | 334 | 342 | VIYQYVDDL |
| 217 | Pol | 336 | 344 | YQYMDDLYV |
| 218 | Pol | 336 | 344 | YQYVDDLYV |
| 219 | Pol | 338 | 346 | YMDDLYVGS |
| 220 | Pol | 338 | 346 | YVDDLYVGS |
| 221 | Pol | 367 | 399 | WGFTTPDKKHQKEPPFLWMGYELHPDKWTVQPI |
| 222 | Pol | 367 | 399 | WGLTTPDKKHQKDPPFLWMGYELHPDRWTVQPI |
| 223 | Pol | 367 | 431 | WGFTTPDKKHQKEPPFLWMGYELHPDKWTVQPIVLPEKDSWTVNDIQKLVGKLNWASQIYPGIKV |
| 224 | Pol | 367 | 431 | WGLTTPDKKHQKDPPFLWMGYELHPDRWTVQPIELPEKESWTVNDIQKLIGKLNWASQIYAGIKV |
| 225 | Pol | 374 | 398 | KKHQKEPPFLWMGYELHPDKWTVQP |
| 226 | Pol | 374 | 398 | KKHQKDPPFLWMGYELHPDRWTVQP |
| 227 | Pol | 380 | 404 | PPFLWMGYELHPDKWTVQPIVLPEK |
| 228 | Pol | 380 | 404 | PPFLWMGYELHPDRWTVQPIELPEK |
| 229 | Pol | 382 | 390 | FLWMGYELH |
| 230 | Pol | 388 | 396 | ELHPDKWTV |
| 231 | Pol | 388 | 396 | ELHPDRWTV |
| 232 | Pol | 399 | 423 | IVLPEKDSWTVNDIQKLVGKLNWAS |
| 233 | Pol | 399 | 423 | IELPEKESWTVNDIQKLIGKLNWAS |
| 234 | Pol | 400 | 424 | VLPEKDSWTVNDIQKLVGKLNWASQ |
| 235 | Pol | 400 | 424 | ELPEKESWTVNDIQKLIGKLNWASQ |
| 236 | Pol | 401 | 431 | LPEKDSWTVNDIQKLVGKLNWASQIYPGIKV |
| 237 | Pol | 401 | 431 | LPEKESWTVNDIQKLIGKLNWASQIYAGIKV |
| 238 | Pol | 406 | 430 | SWTVNDIQKLVGKLNWASQIYPGIK |
| 239 | Pol | 406 | 430 | SWTVNDIQKLIGKLNWASQIYAGIK |
| 240 | Pol | 407 | 415 | WTVNDIQKL |
| 241 | Pol | 408 | 416 | TVNDIQKLV |
| 242 | Pol | 408 | 416 | TVNDIQKLI |
| 243 | Pol | 414 | 422 | KLVGKLNWA |
| 244 | Pol | 414 | 422 | KLIGKLNWA |
| 257 | Pol | 542 | 554 | PKFKLPIQKETWE |
| 258 | Pol | 542 | 554 | PKFRLPIQKETWD |
| 259 | Pol | 542 | 606 | PKFKLPIQKETWETWWTEYWQATWIPEWEFVNTPPLVKLWYQLEKEPIVGAETFYVDGAANRETK |
| 260 | Pol | 542 | 606 | PKFRLPIQKETWDTWWIDYWQATWIPEWEFTNTPPLVKLWYQLETEPIAGVETFYVDGASNRETK |
| 261 | Pol | 553 | 577 | WETWWTEYWQATWIPEWEFVNTPPL |
| 262 | Pol | 553 | 577 | WDTWWTDYWQATWIPEWEFTNTPPL |
| 263 | Pol | 559 | 589 | EYWQATWIPEWEFVNTPPLVKLWYQLEKEPI |
| 264 | Pol | 559 | 589 | DYWQATWIPEWEFTNTPPLVKLWYQLETEPI |
| 265 | Pol | 561 | 569 | WQATWIPEW |
| 266 | Pol | 591 | 606 | GAETFYVDGAANRETK |
| 267 | Pol | 591 | 606 | GVETFYVDGASNRETK |
| 282 | Pol | 683 | 708 | KEKVYLAWVPAHKGIGGNEQVDKLVS |
| 283 | Pol | 683 | 708 | KEKIYLAWVPAHKGIGGNEQIDKLVS |
| 294 | Pol | 747 | 827 | VAKEIVASCDKCQLKGEAMHGQVDCSPGIWQLDCTHLEGKIILVAVHVASGYIEAEVIPAETGQETAYFLLKLAG |
| | | | | RWPVKT |
| 295 | Pol | 747 | 827 | VAKEIVACCDKCQLKGEAIHGQVDCSPGVWQLDCTHLEGKVILVAVHVASGYMEAEVIPTETGQETAYFILKLAG |
| | | | | RWPVTT |
| 296 | Pol | 759 | 783 | QLKGEAMHGQVDCSPGIWQLDCTHL |
| 297 | Pol | 759 | 783 | QLKGEAIHGQVDCSPGVWQLDCTHL |
| 298 | Pol | 767 | 775 | GQVDCSPGI |
| 299 | Pol | 767 | 775 | GQVDCSPGV |
| 300 | Pol | 768 | 792 | QVDCSPGIWQLDCTHLEGKIILVAV |
| 301 | Pol | 768 | 792 | QVDCSPGVWQLDCTHLEGKVILVAV |
| 302 | Pol | 776 | 784 | WQLDCTHLE |
| 305 | Pol | 840 | 919 | TVKAACWWAGIKQEFGIPYNPQSQGVVESMNKELKKIIGQVRDQAEHLKTAVQMAVFIHNFKRKGGIGGYSAGER |
| | | | | IVDII |
| 306 | Pol | 840 | 919 | AVKAACWWAGVKQEFGIPYHPQSQGVVESMNNELKKIIGQIRDQAEQLKTAVQMAVLIHNFKRKGGIGEYSAGER |
| | | | | IIDII |
| 319 | Pol | 931 | 1003 | AITKIQNFRVYYRDSRDPLWKGPAKLLWKGEGAVVIQDNSDIKVVPRRKAKIIRDYGKQMAGDDCVASRQDED |
| 320 | Pol | 931 | 1003 | AITKLQNFRVYYRDNRDPLWKGPARLLWKGEGAVVIQDNSEIKVVPRRKVKIIRDYGKRMAGDDCVAGRQDED |
| 321 | Pol | 932 | 1003 | ITKIQNFRVYYRDSRDPLWKGPAKLLWKGEGAVVIQDNSDIKVVPRRKAKIIRDYGKQMAGDDCVASRQDED |
| 322 | Pol | 932 | 1003 | ITKLQNFRVYYRDNRDPLWKGPARLLWKGEGAVVIQDNSEIKVVPRRKVKIIRDYGKRMAGDDCVAGRQDED |
| 323 | Pol | 940 | 964 | VYYRDSRDPLWKGPAKLLWKGEGAV |
| 324 | Pol | 940 | 964 | VYYRDNRDPLWKGPARLLWKGEGAV |
| 325 | Pol | 947 | 971 | DPLWKGPAKLLWKGEGAVVIQDNSD |
| 326 | Pol | 947 | 971 | DPLWKGPARLLWKGEGAVVIQDNSE |
| 327 | Pol | 948 | 956 | PLWKGPAKL |
| 328 | Pol | 948 | 956 | PLWKGPARL |
| 329 | Pol | 948 | 972 | PLWKGPAKLLWKGEGAVVIQDNSDI |
| 330 | Pol | 948 | 972 | PLWKGPARLLWKGEGAVVIQDNSEI |
| 331 | Pol | 955 | 963 | KLLWKGEGA |
| 332 | Pol | 955 | 963 | RLLWKGEGA |
| 333 | Pol | 956 | 964 | LLWKGEGAV |
| 334 | Pol | 980 | 1003 | AKIIRDYGKQMAGDDCVASRQDED |
| 335 | Pol | 980 | 1003 | VKIIRDYGKRMAGDDCVAGRQDED |
| 336 | Pol | 988 | 996 | KQMAGDDCV |
| 337 | Pol | 988 | 996 | KRMAGDDCV |

TABLE G
polypeptide segments in first iteration fusion polypeptides (e.g., SEQ ID NOs: 345-350)

| SEQ ID | | | | |
|---|---|---|---|---|
| NO: | Gene | Start | End | Sequence |
| 76 | Gag | 31 | 53 | LKHIVWASRELERFAVNPGLLET |
| 77 | Gag | 31 | 53 | LKHLVWASRELERFALNPGLLET |
| 86 | Gag | 128 | 137 | VSQNYPIVQN |
| 87 | Gag | 128 | 137 | VSQNFPIVQN |
| 92 | Gag | 147 | 217 | ISPRTLNAWVKVVEEKAFSPEVIPMFSALSEGATPQDLNTMLNTVGGHQAAMQMLKETINEEAAEWDRLHP |
| 93 | Gag | 147 | 217 | LSPRTLNAWVKVIEEKAFSPEVIPMFTALSEGATPHDLNTMLNTIGGHQAAMQMLKDTINEEAAEWDRVHP |
| 94 | Gag | 147 | 369 | ISPRTLNAWVKVVEEKAFSPEVIPMFSALSEGATPQDLNTMLNTVGGHQAAMQMLKETINEEAAEWDRLHPVHAG |
| | | | | PIAPGQMREPRGSDIAGTTSTLQEQIGWMTNNPPIPVGEIYKRWIILGLNKIVRMYSPTSILDIRQGPKEPFRDY |
| | | | | VDRFYKTLRAEQASQEVKNWMTETLLVQNANPDCKTILKALGPAATLEEMMTACQGVGGPGHKARVLAEAMSQ |
| 95 | Gag | 147 | 369 | LSPRTLNAWVKVIEEKAFSPEVIPMFTALSEGATPHDLNTMLNTIGGHQAAMQMLKDTINEEAAEWDRVHPVHAG |
| | | | | PVAPGQMRDPRGSDIAGSTSTLQEQIAWMTNNPPIPVGDIYKRWIIMGLNKIVRMYSPVSILDIKQGPKEPFRDY |
| | | | | VDRFYRTLRAEQASQDVKNWMTETLLVQNSNPDCKTILKALGPGATLEEMMSACQGVGGPSHKARVLAEAMCQ |
| 96 | Gag | 150 | 158 | RTLNAWVKV |
| 97 | Gag | 175 | 199 | LSEGATPQDLNTMLNTVGGHQAAMQ |
| 98 | Gag | 175 | 199 | LSEGATPHDLNTMLNTIGGHQAAMQ |
| 99 | Gag | 183 | 191 | DLNTMLNTV |
| 100 | Gag | 183 | 191 | DLNTMLNTI |
| 101 | Gag | 225 | 251 | PGQMREPRGSDIAGTTSTLQEQIGWMT |
| 102 | Gag | 225 | 251 | PGQMRDPRGSDIAGSTSTLQEQIAWMT |
| 103 | Gag | 253 | 285 | NPPIPVGEIYKRWIILGLNKIVRMYSPTSILDI |
| 104 | Gag | 253 | 285 | NPPIPVGDIYKRWIIMGLNKIVRMYSPVSILDI |
| 339 | Gag | 257 | 282 | PVGEIYKRWIILGLNKIVRMYSPTSI |
| 340 | Gag | 257 | 282 | PVGDIYKRWIIMGLNKIVRMYSPVSI |
| 105 | Gag | 257 | 290 | PVGEIYKRWIILGLNKIVRMYSPTSILDIRQGPK |
| 106 | Gag | 257 | 290 | PVGDIYKRWIIMGLNKIVRMYSPVSILDIKQGPK |
| 107 | Gag | 265 | 282 | WIILGLNKIVRMYSPTSI |
| 108 | Gag | 265 | 282 | WIIMGLNKIVRMYSPVSI |
| 109 | Gag | 281 | 314 | SILDIRQGPKEPFRDYVDRFYKTLRAEQASQEVK |
| 110 | Gag | 281 | 314 | SILDIKQGPKEPFRDYVDRFYRTLRAEQASQDVK |
| 341 | Gag | 288 | 313 | GPKEPERDYVDRFYKTLRAEQASQEV |
| 342 | Gag | 288 | 313 | GPKEPERDYVDRFYRTLRAEQASQDV |
| 111 | Gag | 288 | 321 | GPKEPERDYVDRFYKTLRAEQASQEVKNWMTETL |
| 112 | Gag | 288 | 321 | GPKEPERDYVDRFYRTLRAEQASQDVKNWMTETL |
| 113 | Gag | 296 | 313 | YVDRFYKTLRAEQASQEV |
| 114 | Gag | 296 | 313 | YVDRFYRTLRAEQASQDV |
| 115 | Gag | 311 | 369 | QEVKNWMTETLLVQNANPDCKTILKALGPAATLEEMMTACQGVGGPGHKARVLAEAMSQ |
| 116 | Gag | 311 | 369 | QDVKNWMTETLLVQNSNPDCKTILKALGPGATLEEMMSACQGVGGPSHKARVLAEAMCQ |
| 117 | Gag | 333 | 357 | ILKALGPAATLEEMMTACQGVGGPG |
| 118 | Gag | 333 | 357 | ILKALGPGATLEEMMSACQGVGGPS |
| 119 | Gag | 337 | 361 | LGPAATLEEMMTACQGVGGPGHKAR |
| 120 | Gag | 337 | 361 | LGPGATLEEMMSACQGVGGPSHKAR |
| 121 | Gag | 341 | 349 | ATLEEMMTA |
| 122 | Gag | 341 | 349 | ATLEEMMSA |
| 123 | Gag | 345 | 353 | EMMTACQGV |
| 124 | Gag | 345 | 353 | EMMSACQGV |
| 149 | Nef | 64 | 82 | EEVGFPVRPQVPLRPMTYK |
| 150 | Nef | 64 | 82 | EEVGFPVKPQVPLRPMTFK |
| 151 | Nef | 64 | 99 | EEVGFPVKPQVPLRPMTFKGALDLSHFLREKGGLEG |
| 152 | Nef | 64 | 99 | EEVGFPVRPQVPLRPMTYKGALDLSHFLKEKGGLEG |
| 180 | Pol | 94 | 117 | LPGRWKPKMIGGIGGFIKVRQYDQ |
| 181 | Pol | 94 | 117 | LPGKWKPKMIGGIGGFIKVKQYDQ |
| 182 | Pol | 129 | 260 | GTVLVGPTPVNIIGRNLLTQIGCTLNFPISPIETVPVKLKPGMDGPKVKQWPLTEEKIKALVEICTEMEKEGKIS |
| | | | | KIGPENPYNTPVFAIKKKDSTKWRKLVDFRELNKRTQDFWEVQLGIPHPAGLKKKKS |
| 183 | Pol | 129 | 260 | GTVLIGPTPVNIIGRNLLTQLGCTLNFPISPIDTVPVKLKPGMDGPRVKQWPLTEEKIKALIEICTEMEKEGKIS |
| | | | | RIGPENPYNTPIFAIKKKDGTKWRKLVDFRELNKKTQDFWEVQLGIPHPSGLKKKKS |
| 184 | Pol | 129 | 277 | GTVLVGPTPVNIIGRNLLTQIGCTLNFPISPIETVPVKLKPGMDGPKVKQWPLTEEKIKALVEICTEMEKEGKIS |
| | | | | KIGPENPYNTPVFAIKKKDSTKWRKLVDFRELNKRTQDFWEVQLGIPHPAGLKKKKSVTVLDVGDAYFSVPLDK |
| 185 | Pol | 129 | 277 | GTVLIGPTPVNIIGRNLLTQLGCTLNFPISPIDTVPVKLKPGMDGPRVKQWPLTEEKIKALIEICTEMEKEGKIS |
| | | | | RIGPENPYNTPIFAIKKKDGTKWRKLVDFRELNKKTQDFWEVQLGIPHPSGLKKKKSVTILDVGDAYFSIPLDK |
| 186 | Pol | 129 | 289 | GTVLVGPTPVNIIGRNLLTQIGCTLNFPISPIETVPVKLKPGMDGPKVKQWPLTEEKIKALVEICTEMEKEGKIS |
| | | | | KIGPENPYNTPVFAIKKKDSTKWRKLVDFRELNKRIQDFWEVQLGIPHPAGLKKKKSVTVLDVGDAYFSVPLDKD |
| | | | | FRKYTAFTIPS |
| 187 | Pol | 129 | 289 | GTVLIGPTPVNIIGRNLLTQLGCTLNFPISPIDTVPVKLKPGMDGPRVKQWPLTEEKIKALIEICTEMEKEGKIS |
| | | | | RIGPENPYNTPIFAIKKKDGTKWRKLVDFRELNKKTQDFWEVQLGIPHPSGLKKKKSVTVLDIGDAYFSVPLDKE |
| | | | | FRKYTAFTVPS |
| 190 | Pol | 144 | 168 | NLLTQIGCTLNFPISPIETVPVKLK |
| 191 | Pol | 144 | 168 | NLLTQLGCTLNFPISPIDTVPVKLK |
| 192 | Pol | 152 | 160 | TLNFPISPI |
| 193 | Pol | 254 | 277 | GLKKKKSVTVLDVGDAYFSVPLDK |
| 194 | Pol | 254 | 277 | GLKKNKSVTVLDVGDAYFSIPLDK |
| 195 | Pol | 278 | 289 | DFRKYTAFTIPS |
| 196 | Pol | 278 | 289 | EFRKYTAFTVPS |
| 221 | Pol | 367 | 399 | WGFTTPDKKHQKEPPFLWMGYELHPDKWTVQPI |
| 222 | Pol | 367 | 399 | WGLTTPDKKHQKDPPFLWMGYELHPDRWTVQPI |
| 294 | Pol | 747 | 827 | VAKEIVASCDKCQLKGEAMHGQVDCSPGIWQLDCTHLEGKIILVAVHVASGYIEAEVIPAETGQETAYFLLKLAG |
| | | | | RWPVKT |
| 295 | Pol | 747 | 827 | VAKEIVACCDKCQLKGEAIHGQVDCSPGVWQLDCTHLEGKVILVAVHVASGYMEAEVIPTETGQETAYFILKLAG |
| | | | | RWPVTT |
| 296 | Pol | 759 | 783 | QLKGEAMHGQVDCSPGIWQLDCTHL |
| 297 | Pol | 759 | 783 | QLKGEAIHGQVDCSPGVWQLDCTHL |
| 298 | Pol | 767 | 775 | GQVDCSPGI |
| 299 | Pol | 767 | 775 | GQVDCSPGV |
| 300 | Pol | 768 | 792 | QVDCSPGIWQLDCTHLEGKIILVAV |
| 301 | Pol | 768 | 792 | QVDCSPGVWQLDCTHLEGKVILVAV |
| 305 | Pol | 840 | 919 | TVKAACWWAGIKQEFGIPYNPQSQGVVESMNKELKKIIGQVRDQAEHLKTAVQMAVFIHNFKRKGGIGGYSAGER |
| | | | | IVDII |
| 306 | Pol | 840 | 919 | AVKAACWWAGVKQEFGIPYHPQSQGVVESMNNELKKIIGQIRDQAEQLKTAVQMAVLIHNFKRKGGIGEYSAGER |
| | | | | IIDII |
| 307 | Pol | 840 | 920 | TVKAACWWAGIKQEFGIPYNPQSQGVVESMNKELKKIIGQVRDQAEHLKTAVQMAVFIHNFKRKGGIGGYSAGER |
| | | | | IVDIIA |
| 308 | Pol | 840 | 920 | AVKAACWWAGVKQEFGIPYHPQSQGVVESMNNELKKIIGQIRDQAEQLKTAVQMAVLIHNFKRKGGIGEYSAGER |
| | | | | IIDIIA |
| 311 | Pol | 842 | 850 | KAACWWAGI |
| 312 | Pol | 842 | 850 | KAACWWAGV |
| 321 | Pol | 932 | 1003 | ITKIQNFRVYYRDSRDPLWKGPAKLLWKGEGAVVIQDNSDIKVVPRRKAKIIRDYGKQMAGDDCVASRQDED |
| 322 | Pol | 932 | 1003 | ITKLQNFRVYYRDNRDPLWKGPARLLWKGEGAVVIQDNSEIKVVPRRKVKIIRDYGKRMAGDDCVAGRQDED |
| 323 | Pol | 940 | 964 | VYYRDSRDPLWKGPAKLLWKGEGAV |
| 324 | Pol | 940 | 964 | VYYRDNRDPLWKGPARLLWKGEGAV |
| 325 | Pol | 947 | 971 | DPLWKGPAKLLWKGEGAVVIQDNSD |
| 326 | Pol | 947 | 971 | DPLWKGPARLLWKGEGAVVIQDNSE |
| 327 | Pol | 948 | 956 | PLWKGPAKL |
| 328 | Pol | 948 | 956 | PLWKGPARL |
| 329 | Pol | 948 | 972 | PLWKGPAKLLWKGEGAVVIQDNSDI |
| 330 | Pol | 948 | 972 | PLWKGPARLLWKGEGAVVIQDNSEI |
| 331 | Pol | 955 | 963 | KLLWKGEGA |
| 332 | Pol | 955 | 963 | RLLWKGEGA |
| 333 | Pol | 956 | 964 | LLWKGEGAV |
| 334 | Pol | 980 | 1003 | AKIIRDYGKQMAGDDCVASRQDED |
| 335 | Pol | 980 | 1003 | VKIIRDYGKRMAGDDCVAGRQDED |
| 336 | Pol | 988 | 996 | KQMAGDDCV |
| 337 | Pol | 988 | 996 | KRMAGDDCV |

### Fusion Polypeptides Having Polypeptide Segments Encoded by HIV-1 Env, Gag, Nef and Pol, and Predicted to Bind to or be Presented by Human HLA A*0201 Molecules

As described herein, we have designed fusion polypeptides having a plurality of polypeptide segments that have been computationally predicted to bind to or be presented by human HLA A*0201 molecules. Generally, the selected or included polypeptide segments in such fusion polypeptides comprise polypeptide segments encoded by HIV-1 Env, Gag, Nef and Pol genes (e.g., do not comprise one or more polypeptide segments encoded by the HIV-1 Tat, Rev, Vif, Vpr or Vpu genes), and which are predicted to bind to a human HLA A*0201 molecule with an IC50 value of less than about 1,000 nM. In certain embodiments, the selected or included polypeptide segments in such fusion polypeptides comprise polypeptide segments encoded by HIV-1 Env, Gag, Nef and Pol genes (e.g., do not comprise one or more polypeptide segments encoded by the HIV-1 Tat, Rev, Vif, Vpr or Vpu genes), and which are predicted to bind to a human HLA A*0201 molecule with a percentile rank within the top 5% in a population of polypeptide segments. In some embodiments, the fusion polypeptide comprises or consists of segments of viral proteins encoded by Gag, Pol, Env, and Nef genes, wherein each of the plurality of polypeptide segments can bind to or be presented by a human HLA allele A*0201.

In some embodiments, each of the plurality of polypeptide segments are from 8-35 amino acids in length, e.g. from 9-34 amino acids in length, e.g. from 9-25 amino acids in length.

In some embodiments, the fusion polypeptide comprises at least 2 polypeptide segments, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, or more, segments comprising or consisting of an amino acid sequence selected from: SEQ ID NOs: 6, 7, 15, 16, 21, 22, 30, 60, 61, 78, 79, 96, 99, 100, 107, 108, 113, 114, 121, 122, 123, 124, 137, 138, 153, 154, 172, 173, 192, 201, 202, 215, 216, 217, 218, 219, 220, 229, 230, 231, 240, 241, 242, 243, 244, 265, 276, 277, 298, 299, 302, 311, 312, 327, 328, 331, 332, 333, 336, and 337; SEQ ID NOs: 6, 15, 21, 30, 60, 78, 99, 107, 113, 121, 123, 137, 153, 172, 192, 201, 215, 217, 219, 229, 230, 240, 241, 243, 265, 276, 298, 302, 311, 327, 331, 333 and 336; SEQ ID NOs: 1, 2, 3, 4, 5, 6, 7, 10, 11, 12, 13, 14, 15, 16, 19, 20, 27, 55, 56, 57, 58, 59, 60, 61, 78, 79, 90, 91, 97, 98, 99, 100, 105, 106, 107, 108, 111, 112, 113, 114, 117, 118, 119, 120, 121, 122, 123, 124, 137, 138, 153, 154, 155, 156, 157, 158, 163, 164, 165, 166, 167, 168, 169, 170, 171, 172, 173, 190, 191, 192, 197, 198, 209, 210, 211, 212, 213, 214, 215, 216, 217, 218, 219, 220, 225, 226, 227, 228, 229, 230, 231, 232, 233, 234, 235, 238, 239, 261, 262, 274, 275, 276, 277, 296, 297, 298, 299, 300, 301, 302, 303, 304, 323, 324, 325, 326, 327, 328, 329, 330, 331, 332, 333, 334, 335, 336, 337, 338, 343 and 344; SEQ ID NOs: 1, 10, 19, 27, 55, 56, 57, 78, 90, 97, 105, 111, 117, 119, 137, 153, 165, 190, 197, 209, 210, 211, 225, 227, 234, 238, 261, 296, 300, 303, 323, 325, 329 and 334. Polypeptide segments included in fusion polypeptides having polypeptide segments encoded by HIV-1 Env, Gag, Nef and Pol genes, and predicted to bind to or be presented by human HLA A*0201 molecules are listed in Table H.

TABLE H
polypeptide segments in A*0201 binding fusion
polypeptides (e.g., SEQ ID NOs: 367-377, 411)

| SEQ | | | | |
|---|---|---|---|---|
| ID | | | | |
| NO: | Gene | Start | End | Sequence |
| 1 | Env | 28 | 52 | CSATEKLWVTVYYGVPVWKEATTTL |
| 2 | Env | 34 | 48 | LWVTVYYGVPVWKEA |
| 3 | Env | 34 | 48 | LWVTIYYGVPVWKDA |
| 4 | Env | 34 | 47 | LWVTVYYGVPVWKE |
| 5 | Env | 34 | 47 | LWVTIYYGVPVWKD |
| 6 | Env | 36 | 44 | VTVYYGVPV |
| 7 | Env | 36 | 44 | VTIYYGVPV |
| 10 | Env | 59 | 83 | KAYDIEVHNVWATHACVPTDPNPQE |
| 11 | Env | 64 | 83 | AHNVWATHACVPTDPNPQE |
| 12 | Env | 64 | 83 | VHNIWATHACVPTDPSPQE |
| 13 | Env | 66 | 83 | HNVWATHACVPTDPNPQE |
| 14 | Env | 66 | 83 | HNIWATHACVPTDPSPQE |
| 15 | Env | 67 | 75 | NVWATHACV |
| 16 | Env | 67 | 75 | NIWATHACV |
| 19 | Env | 113 | 137 | DQSLKPCVKLTPLCVTLNCTDLRNT |
| 20 | Env | 113 | 137 | DESLKPCVKLTPICVTLNCTDLRNT |
| 27 | Env | 235 | 259 | GTGPCTNVSTVQCTHGIRPVVSTQL |
| 55 | Env | 586 | 594 | YLKDQQLLG |
| 56 | Env | 586 | 594 | YLRDQQLLG |
| 57 | Env | 586 | 610 | YLKDQQLLGIWGCSGKLICTTAVPW |
| 338 | Env | 586 | 610 | YLRDQQLLGLWGCSGKLICPTAVPW |
| 58 | Env | 589 | 606 | DQQLLGIWGCSGKLICTT |
| 59 | Env | 589 | 606 | DQQLLGLWGCSGKLICPT |
| 60 | Env | 594 | 602 | GIWGCSGKL |
| 61 | Env | 594 | 602 | GLWGCSGKL |
| 78 | Gag | 37 | 51 | ASRELERFAVNPGLL |
| 79 | Gag | 37 | 51 | ASRELERFALNPGLL |
| 90 | Gag | 142 | 166 | MVHQAISPRTLNAWVKVVEEKAFSP |
| 91 | Gag | 142 | 166 | MVHQPISPRTLNAWVKVIEEKAFSP |
| 97 | Gag | 175 | 199 | LSEGATPQDLNTMLNTVGGHQAAMQ |
| 98 | Gag | 175 | 199 | LSEGATPHDLNTMLNTIGGHQAAMQ |
| 99 | Gag | 183 | 191 | DLNTMLNTV |
| 100 | Gag | 183 | 191 | DLNTMLNTI |
| 339 | Gag | 257 | 282 | PVGEIYKRWIILGLNKIVRMYSPTSI |
| 340 | Gag | 257 | 282 | PVGDIYKRWIIMGLNKIVRMYSPVSI |
| 107 | Gag | 265 | 282 | WIILGLNKIVRMYSPTSI |
| 108 | Gag | 265 | 282 | WIIMGLNKIVRMYSPVSI |
| 341 | Gag | 288 | 313 | GPKEPFRDYVDRFYKTLRAEQASQEV |
| 342 | Gag | 288 | 313 | GPKEPFRDYVDRFYRTLRAEQASQDV |
| 113 | Gag | 296 | 313 | YVDRFYKTLRAEQASQEV |
| 114 | Gag | 296 | 313 | YVDRFYRTLRAEQASQDV |
| 117 | Gag | 333 | 357 | ILKALGPAATLEEMMTACQGVGGPG |
| 118 | Gag | 333 | 357 | ILKALGPGATLEEMMSACQGVGGPS |
| 119 | Gag | 337 | 361 | LGPAATLEEMMTACQGVGGPGHKAR |
| 120 | Gag | 337 | 361 | LGPGATLEEMMSACQGVGGPSHKAR |
| 121 | Gag | 341 | 349 | ATLEEMMTA |
| 122 | Gag | 341 | 349 | ATLEEMMSA |
| 123 | Gag | 345 | 353 | EMMTACQGV |
| 124 | Gag | 345 | 353 | EMMSACQGV |
| 137 | Gag | 429 | 444 | RQANFLGKIWPSHKGR |
| 138 | Gag | 429 | 444 | RQANFLGKVWPSHNGR |
| 153 | Nef | 81 | 102 | YKAAVDLSHFLREKGGLEGAAY |
| 154 | Nef | 81 | 102 | YKGALDLSHFLKEKGGLEGAAY |
| 155 | Nef | 88 | 97 | SHFLKEKGGL |
| 156 | Nef | 88 | 97 | SHFLREKGGL |
| 157 | Nef | 91 | 99 | LKEKGGLEG |
| 158 | Nef | 91 | 99 | LREKGGLEG |
| 163 | Nef | 130 | 148 | EPGIRFPLTFGWCFKLVPL |
| 164 | Nef | 130 | 148 | GPGTRYPLTFGWCFKLVPV |
| 165 | Nef | 130 | 154 | GPGIRYPLLTFGWCFKLPVEPEKVE |
| 166 | Nef | 134 | 142 | RYPLTFGWC |
| 167 | Nef | 134 | 142 | RFPLTFGWC |
| 168 | Nef | 134 | 148 | RYPLTFGWCFKLVPV |
| 169 | Nef | 134 | 148 | RFPLTFGWCFKLVPL |
| 170 | Nef | 136 | 148 | PLTFGWCFKLVPV |
| 171 | Nef | 136 | 148 | PLCFGWCFKLVPL |
| 172 | Nef | 137 | 145 | LTFGWCFKL |
| 173 | Nef | 137 | 145 | LCFGWCFKL |
| 190 | Pol | 144 | 168 | NLLTQIGCTLNFPISPIETVPVKLK |
| 191 | Pol | 144 | 168 | NLLTQLGCTLNFPISPIDTVPVKLK |
| 192 | Pol | 152 | 160 | TLNFPISPI |
| 197 | Pol | 291 | 315 | NNETPGIRYQYNVLPQGWKGSPAIF |
| 198 | Pol | 291 | 315 | NNETPGVRYQYNVLPMGWKGSPAIF |
| 209 | Pol | 326 | 350 | FRKQNPDIVIYQYMDDLYVGSDLEI |
| 343 | Pol | 326 | 350 | FRKQNPDIVIYQYVDDLYVGSDLEI |
| 210 | Pol | 328 | 352 | KQNPDIVIYQYMDDLYVGSDLEIGQ |
| 344 | Pol | 328 | 352 | KQNPDIVIYQYVDDLYVGSDLEIEQ |
| 211 | Pol | 330 | 354 | NPDIVIYQYMDDLYVGSDLEIGQHR |
| 212 | Pol | 330 | 354 | NPDIVIYQYVDDLYVGSDLEIEQHR |
| 213 | Pol | 333 | 354 | IVIYQYMDDLYVGSDLEIGQHR |
| 214 | Pol | 333 | 354 | IVIYQYVDDLYVGSDLEIEQHR |
| 215 | Pol | 334 | 342 | VIYQYMDDL |
| 216 | Pol | 334 | 342 | VIYQYVDDL |
| 217 | Pol | 336 | 344 | YQYMDDLYV |
| 218 | Pol | 336 | 344 | YQYVDDLYV |
| 219 | Pol | 338 | 346 | YMDDLYVGS |
| 220 | Pol | 338 | 346 | YVDDLYVGS |
| 225 | Pol | 374 | 398 | KKHQKEPPFLWMGYELHPDKWTVQP |
| 226 | Pol | 374 | 398 | KKHQKDPPFLWMGYELHPDRWTVQP |
| 227 | Pol | 380 | 404 | PPFLWMGYELHPDKWTVQPIVLPEK |
| 228 | Pol | 380 | 404 | PPFLWMGYELHPDRWTVQPIELPEK |
| 229 | Pol | 382 | 390 | FLWMGYELH |
| 230 | Pol | 388 | 396 | ELHPDKWTV |
| 231 | Pol | 388 | 396 | ELHPDRWTV |
| 232 | Pol | 399 | 423 | IVLPEKDSWTVNDIQKLVGKLNWAS |
| 233 | Pol | 399 | 423 | IELPEKESWTVNDIQKLIGKLNWAS |
| 234 | Pol | 400 | 424 | VLPEKDSWTVNDIQKLVGKLNWASQ |
| 235 | Pol | 400 | 424 | ELPEKESWTVNDIQKLIGKLNWASQ |
| 238 | Pol | 406 | 430 | SWTVNDIQKLVGKLNWASQIYPGIK |
| 239 | Pol | 406 | 430 | SWTVNDIQKLIGKLNWASQIYAGIK |
| 261 | Pol | 553 | 577 | WETWWTEYWQATWIPEWEFVNTPPL |
| 262 | Pol | 553 | 577 | WDTWWTDYWQATWIPEWEFTNTPPL |
| 274 | Pol | 642 | 666 | QDSGLEVNIVTDSQYALGIIQAQPD |
| 275 | Pol | 642 | 666 | QDSGSEVNIVTDSQYAIGIIQAQPD |
| 276 | Pol | 650 | 658 | IVTDSQYAL |
| 277 | Pol | 650 | 658 | IVTDSQYAI |
| 296 | Pol | 759 | 783 | QLKGEAMHGQVDCSPGIWQLDCTHL |
| 297 | Pol | 759 | 783 | QLKGEAIHGQVDCSPGVWQLDCTHL |
| 298 | Pol | 767 | 775 | GQVDCSPGI |
| 299 | Pol | 767 | 775 | GQVDCSPGV |
| 300 | Pol | 768 | 792 | QVDCSPGIWQLDCTHLEGKIILVAV |
| 301 | Pol | 768 | 792 | QVDCSPGVWQLDCTHLEGKVILVAV |
| 302 | Pol | 776 | 784 | WQLDCTHLE |
| 303 | Pol | 834 | 858 | SNFTSTIVKAACWWAGIKQEFGIPY |
| 304 | Pol | 834 | 858 | SNFTSTAVKAACWWAGVKQEFGIPY |
| 323 | Pol | 940 | 964 | VYYRDSRDPLWKGPAKLLWKGEGAV |
| 324 | Pol | 940 | 964 | VYYRDNRDPLWKGPARLLWKGEGAV |
| 325 | Pol | 947 | 971 | DPLWKGPAKLLWKGEGAVVIQDNSD |
| 326 | Pol | 947 | 971 | DPLWKGPARLLWKGEGAVVIQDNSE |
| 327 | Pol | 948 | 956 | PLWKGPAKL |
| 328 | Pol | 948 | 956 | PLWKGPARL |
| 329 | Pol | 948 | 972 | PLWKGPAKLLWKGEGAVVIQDNSDI |
| 330 | Pol | 948 | 972 | PLWKGPARLLWKGEGAVVIQDNSEI |
| 331 | Pol | 955 | 963 | KLLWKGEGA |
| 332 | Pol | 955 | 963 | RLLWKGEGA |
| 333 | Pol | 956 | 964 | LLWKGEGAV |
| 334 | Pol | 980 | 1003 | AKIIRDYGKQMAGDDCVASRQDED |
| 335 | Pol | 980 | 1003 | VKIIRDYGKRMAGDDCVAGRQDED |
| 336 | Pol | 988 | 996 | KQMAGDDCV |
| 337 | Pol | 988 | 996 | KRMAGDDCV |

In some embodiments, the fusion polypeptide comprises the following polypeptide segments in sequential order, from N-terminus to C-terminus, optionally joined or connected by one or more linkers: SEQ ID NOs: 201, 78, 107, 96, 229, 172, 327, 6, 333, 243, 331, 192, 265, 311, 137, 15, 123, 30, 336, 302, 153, 219, 298, 121, 230, 240, 60, 241, 276, 113, 99, 21, 217 and 215; SEQ ID NOs: 78, 296, 1, 339, 197, 329, 232, 323, 303, 234, 90, 261, 274, 238, 211, 325, 137, 227, 209, 190, 341, 57, 225, 27, 210, 119, 19, 165, 334, 117, 153, 10, 97 and 300; or SEQ ID NOs: 296, 1, 78, 197, 339, 227, 261, 274, 238, 325, 137, 329, 303, 234, 90, 232, 27, 57, 225, 323, 190, 341, 119, 19, 165, 334, 117, 153, 10, 97 and 300.

In some embodiments, the fusion polypeptide comprises an amino acid sequence of any one of SEQ ID NOs: 367-377 and 411, or a sequence that is 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 367-377 and 411.

### Signal or Leader Sequences

In various embodiments, the fusion polypeptides comprise a signal sequence or signal peptide, e.g., to direct intracellular trafficking of the fusion polypeptide to a proteasomal or lysosomal compartment. In various embodiments, fusion polypeptide comprises a signal sequence at the N-terminus and/or the C-terminus. In some embodiments, the fusion polypeptide comprises an N-terminal signal peptide or leader sequence. In various embodiments, the signal peptide or leader sequence is from a source protein selected from a serum protein, a cytokine, a chemokine, a chaperone protein, an invariant protein, and a protein that directs proteins to the lysosomal compartment. In some embodiments, the signal peptide or leader sequence is from a source protein selected from the group consisting of colony stimulating factor 2 (CSF2, GM-CSF), tissue type plasminogen activator (PLAT, t-PA), C-C motif chemokine ligand 7 (CCL7, MCP-3), C-X-C motif chemokine ligand 10 (CXCL10, IP-10), catenin beta 1 (CTNNB1), CD74 (p33; DHLAG; HLADG; Ia-GAMMA, invariant chain), serum albumin (ALB), polyubiquitin B/C (UBB/UBC), calreticulin (CALR), vesicular stomatitis virus G protein (VSV-G), lysosomal associated membrane protein 1 (LAMP-1) and lysosomal associated membrane protein 2 (LAMP-2). In certain embodiments, the fusion polypeptide comprises N-terminal and C-terminal signal sequences from LAMP-1, e.g, SEQ ID NOs: 399 and 412, respectively. In various embodiments, the signal peptide or leader sequence is selected from an amino acid sequence of any one of SEQ ID NOs: 393-402 and 412-413, or a sequence that is at least 95%, 96%, 97%, 98%, or 99% identical to any one of SEQ ID NOs: 393-402 and 412-413. Illustrative signal sequences that can be used in the present fusion polypeptides are provided in Table I.

TABLE I
signal sequences

| SEQ | | |
|---|---|---|
| ID | source | |
| NO: | protein name | SEQUENCE |
| 393 | CSF2, GM-CSF | MWLQSLLLLGTVACSISV |
| 394 | PLAT, t-PA | MDAMKRGLCCVLLLCGAVFVSAR |
| 395 | CD74 | MHRRRSRSCREDQKPV |
| 396 | albumin | KWVTFISLLFLFSSAYS |
| 397 | p-catenin | MRKAAVSHWQQQSYLDSGIHSGATTTAPSLS |
| 398 | CCL7, MCP-3 | MNPSAAVIFCLILLGLSGTQGILDMAQPVGI |
| | | NTSTTCCYRFINKKIPKQRLESYRRTTSSHC |
| | | PREAVIFKTKLDKEICADPTQKWVQDFMKHL |
| | | DKKTQTPKLASAGA |
| 399 | LA4P-1 | MAPRSARRPLLLLLLLLLLGLMHCASAAMFM |
| | N-terminal | VKNGNGTACIMANFSAAFSVNYDTKSGPKNM |
| | | TLDLPSDATVVLNRSSCGKENTSDPSLVIAF |
| | | GRGHTLTLNFTRNATRYSVQLMSFVYNLSDT |
| | | HLFPNASSKEIKTVESITDIRADIDKKYRCV |
| | | SGTQVHMNNVTVTLHDATIQAYLSNSSFSRG |
| | | ETRCEQDRPSPTTAPPAPPSPSPSPVPKSPS |
| | | VDKYNVSGTNGTCLLASMGLQLNLTYERKDN |
| | | TTVTRLLNINPNKTSASGSCGAHLVTLELHS |
| | | EGTTVLLFQFGMNASSSRFFLQGIQLNTlLP |
| | | DARDPAFKAANGSLRALQATVGNSYKCNAEE |
| | | HVRVTKAFSVNIFKVWVQAFKVEGGQFGSVE |
| | | ECLLDENSLEDI |
| 412 | LAMP-1 | GSEFTLIPIAVGGALAGLVIVLIAYLVGRKR |
| | C-terminal | SHAGYQTI |
| 400 | ubiquitin | MQIFVKTLTGKTITLEVEPSDTIENVKAKIQ |
| | | DKEGIPPDQQRLIFAGKQLEDGRTLSDYNIQ |
| | | KESTLHLVLRLRGG |
| 401 | calreticulin | MLLSVPLLLGLLGLAVA |
| 402 | VSV-G | MKCLLYLAFLFIGVNC |
| 413 | CXCL10, | MNQTAILICCLIFLTLSGIQG |
| | IP-10 | |

Illustrative fusion polypeptides, with and without signal sequences, which have been designed and assembled according to the herein described methods, are provided in Table J.

In various embodiments, the fusion polypeptides described herein do not comprise 1, 2, 3, 4, 5, or more, or any or all, polypeptide segments comprising or consisting of an HIV-1 amino acid sequence of SEQ ID NOs: 437-461, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to an amino acid sequence of SEQ ID NOs: 437-461, or subsequences thereof. Amino acid sequences that can be excluded from (i.e., not included in) the herein described fusion polypeptides in certain embodiments are provided in Table K.

Further provided are methods for making a fusion polypeptide, pharmaceutical composition, immunogenic composition or vaccine composition comprising same. In some implementations, the methods comprise constructing the fusion polypeptides using peptide synthesis. In some implementations, the methods comprise constructing, using synthetic or recombinant DNA technology, polynucleotides encoding each of the polypeptides of the bivalent antigen and expressing the polypeptides from an expression vector. In some implementations, the methods may further comprise inserting the polynucleotides into one or more vectors and expressing the encoded polypeptides in a cell.

TABLE J
immunogenic fusion polypeptides comprising HIV-1 polypeptide segments (“AAA” is SEQ ID NO:
378, “AAY” is SEQ ID NO: 379, “YMDD” is SEQ ID NO: 462 and “REKR” is SEQ ID NO: 382)

| SEQ | Linker/ | | |
|---|---|---|---|
| ID | Signal | HIV-1 | |
| NO: | peptide | Genes | SEQUENCE |
| 345 | AAA/ | Gag, | LKHIVWASRELERFAVNPGLLETAAAVSQNYPIVQNAAAISPRTLNAWVKVVEEKAFSPEVIPMFSALSEGATPQDLNTMLN |
| | none | Pol, | TVGGHQAAMQMLKETINEEAAEWDRLHPVHAGPIAPGQMREPRGSDIAGTTSTLQEQIGWMTNNPPIPVGEIYKRWIILGLN |
| | | Nef | KIVRMYSPTSILDIRQGPKEPFRDYVDRFYKTLRAEQASQEVKNWMTETLLVQNANPDCKTILKALGPAATLEEMMTACQGV |
| | | | GGPGHKARVLAEAMSQAAALPGRWKPKMIGGIGGFIKVRQYDQAAAGTVLVGPTPVNIIGRNLLTQIGCTLNFPISPIETVP |
| | | | VKLKPGMDGPKVKQWPLTEEKIKALVEICTEMEKEGKISKIGPENPYNTPVFAIKKKDSTKWRKLVDFRELNKRTQDFWEVQ |
| | | | LGIPHPAGLKKKKSVTVLDVGDAYFSVPLDKDFRKYTAFTIPSAAAWGFTTPDKKHQKEPPFLWMGYELHPDKWIVQPIAAA |
| | | | VAKEIVASCDKCQLKGEAMHGQVDCSPGIWQLDCTHLEGKIILVAVHVASGYIEAEVIPAETGQETAYFLLKLAGRWPVKTA |
| | | | AATVKAACWWAGIKQEFGIPYNPQSQGVVESMNKELKKIIGQVRDQAEHLKTAVQMAVFIHNFKRKGGIGGYSAGERIVDII |
| | | | AAAAITKIQNFRVYYRDSRDPLWKGPAKLLWKGEGAVVIQDNSDIKVVPRRKAKIIRDYGKQMAGDDCVASRQDEDAAAEEV |
| | | | GFPVKPQVPLRPMTFKGALDLSHFLREKGGLEG |
| 346 | AAA/ | Gag, | LKHLVWASRELERFALNPGLLETAAAVSQNFPIVQNAAALSPRILNAWVKVIEEKAFSPEVIPMFTALSEGATPHDLNTMLN |
| | none | Pol, | TIGGHQAAMQMLKDTINEEAAEWDRVHPVHAGPVAPGQMRDPRGSDIAGSTSTLQEQIAWMTNNPPIPVGDIYKRWIIMGLN |
| | | Nef | KIVRMYSPVSILDIKQGPKEPFRDYVDRFYRTLRAEQASQDVKNWMTETLLVQNSNPDCKTILKALGPGATLEEMMSACQGV |
| | | | GGPSHKARVLAEAMCQAAALPGKWKPKMIGGIGGFIKVKQYDQAAAGTVLIGPTPVNIIGRNLLTQLGCTLNFPISPIDTVP |
| | | | VKLKPGMDGPRVKQWPLTEEKIKALIEICTEMEKEGKISRIGPENPYNTPIFAIKKKDGTKWRKLVDFRELNKKTQDFWEVQ |
| | | | LGIPHPSGLKKKKSVTVLDIGDAYFSVPLDKEFRKYTAFTVPSAAAWGLTTPDKKHQKDPPFLWMGYELHPDRWTVQPIAAA |
| | | | VAKEIVACCDKCQLKGEAIHGQVDCSPGVWQLDCTHLEGKVILVAVHVASGYMEAEVIPTETGQETAYFILKLAGRWPVTTA |
| | | | AAAVKAACWWAGVKQEFGIPYHPQSQGVVESMNNELKKIIGQIRDQAEQLKTAVQMAVLIHNFKRKGGIGEYSAGERIIDII |
| | | | AAAAITKLQNFRVYYRDNRDPLWKGPARLLWKGEGAVVIQDNSEIKVVPRRKVKIIRDYGKRMAGDDCVAGRQDEDAAAEEV |
| | | | GFPVRPQVPLRPMTYKGALDLSHFLKEKGGLEG |
| 347 | F2A/ | Gag, | LKHIVWASRELERFAVNPGLLETVSQNYPIVQNISPRTLNAWVKVVEEKAFSPEVIPMFSALSEGATPQDLNTMLNTVGGHQ |
| | none | Pol, | AAMQMLKETINEEAAEWDRLHPVHAGPIAPGQMREPRGSDIAGTTSTLQEQIGWMTNNPPIPVGEIYKRWIILGLNKIVRMY |
| | | Nef | SPTSILDIRQGPKEPFRDYVDRFYKTLRAEQASQEVKNWMTETLLVQNANPDCKTILKALGPAATLEEMMTACQGVGGPGHK |
| | | | ARVLAEAMSQRAKRAPVKQTLNFDLLKLAGDVESNPGPLPGRWKPKMIGGIGGFIKVRQYDQGTVLVGPTPVNIIGRNLLTQ |
| | | | IGCTLNFPISPIETVPVKLKPGMDGPKVKQWPLTEEKIKALVEICTEMEKEGKISKIGPENPYNTPVFAIKKKDSTKWRKLV |
| | | | DFRELNKRTQDFWEVQLGIPHPAGLKKKKSVTVLDVGDAYFSVPLDKDFRKYTAFTIPSWGFTTPDKKHQKEPPFLWMGYEL |
| | | | HPDKWTVQPIVAKEIVASCDKCQLKGEAMHGQVDCSPGIWQLDCTHLEGKIILVAVHVASGYIEAEVIPAETGQETAYFLLK |
| | | | LAGRWPVKTTVKAACWWAGIKQEFGIPYNPQSQGVVESMNKELKKIIGQVRDQAEHLKTAVQMAVFIHNFKRKGGIGGYSAG |
| | | | ERIVDIIAITKIQNFRVYYRDSRDPLWKGPAKLLWKGEGAVVIQDNSDIKVVPRRKAKIIRDYGKQMAGDDCVASRQDEDRA |
| | | | KRAPVKQTLNFDLLKLAGDVESNPGPEEVGFPVKPQVPLRPMTFKGALDLSHFLREKGGLEG |
| 348 | F2A/ | Gag, | LKHLVWASRELERFALNPGLLETVSQNFPIVQNLSPRTLNAWVKVIEEKAFSPEVIPMFTALSEGATPHDLNTMLNTIGGHQ |
| | none | Pol, | AAMQMLKDTINEEAAEWDRVHPVHAGPVAPGQMRDPRGSDIAGSTSTLQEQIAWMTNNPPIPVGDIYKRWIIMGLNKIVRMY |
| | | Nef | SPVSILDIKQGPKEPFRDYVDRFYRTLRAEQASQDVKNWMTETLLVQNSNPDCKTILKALGPGATLEEMMSACQGVGGPSHK |
| | | | ARVLAEAMCQRAKRAPVKQTLNFDLLKLAGDVESNPGPLPGKWKPKMIGGIGGFIKVKQYDQGTVLIGPTPVNIIGRNLLTQ |
| | | | LGCTLNFPISPIDTVPVKLKPGMDGPRVKQWPLTEEKIKALIEICTEMEKEGKISRIGPENPYNTPIFAIKKKDGIKWRKLV |
| | | | DFRELNKKTQDFWEVQLGIPHPSGLKKKKSVTVLDIGDAYFSVPLDKEFRKYTAFTVPSWGLTTPDKKHQKDPPFLWMGYEL |
| | | | HPDRWTVQPIVAKEIVACCDKCQLKGEAIHGQVDCSPGVWQLDCTHLEGKVILVAVHVASGYMEAEVIPTETGQETAYFILK |
| | | | LAGRWPVTTAVKAACWWAGVKQEFGIPYHPQSQGVVESMNNELKKIIGQIRDQAEQLKTAVQMAVLIHNFKRKGGIGEYSAG |
| | | | ERIIDIIAITKLQNFRVYYRDNRDPLWKGPARLLWKGEGAVVIQDNSEIKVVPRRKVKIIRDYGKRMAGDDCVAGRQDEDRA |
| | | | KRAPVKQTLNFDLLKLAGDVESNPGPEEVGFPVRPQVPLRPMTYKGALDLSHFLKEKGGLEG |
| 349 | Fusion/ | Gag, | LKHIVWASRELERFAVNPGLLETVSQNYPIVQNISPRTLNAWVKVVEEKAFSPEVIPMFSALSEGATPQDLNTMLNIVGGHQ |
| | none | Pol, | AAMQMLKETINEEAAEWDRLHPVHAGPIAPGQMREPRGSDIAGTTSTLQEQIGWMTNNPPIPVGEIYKRWIILGLNKIVRMY |
| | | Nef | SPTSILDIRQGPKEPFRDYVDRFYKTLRAEQASQEVKNWMTETLLVQNANPDCKTILKALGPAATLEEMMTACQGVGGPGHK |
| | | | ARVLAEAMSQLPGRWKPKMIGGIGGFIKVRQYDQGTVLVGPTPVNIIGRNLLTQIGCTLNFPISPIETVPVKLKPGMDGPKV |
| | | | KQWPLTEEKIKALVEICTEMEKEGKISKIGPENPYNTPVFAIKKKDSTKWRKLVDFRELNKRTQDFWEVQLGIPHPAGLKKK |
| | | | KSVTVLDVGDAYFSVPLDKDFRKYTAFTIPSWGFTTPDKKHQKEPPFLWMGYELHPDKWTVQPIVAKEIVASCDKCQLKGEA |
| | | | MHGQVDCSPGIWQLDCTHLEGKIILVAVHVASGYIEAEVIPAETGQETAYFLLKLAGRWPVKTIVKAACWWAGIKQEFGIPY |
| | | | NPQSQGVVESMNKELKKIIGQVRDQAEHLKTAVQMAVFIHNFKRKGGIGGYSAGERIVDIIAITKIQNFRVYYRDSRDPLWK |
| | | | GPAKLLWKGEGAVVIQDNSDIKVVPRRKAKIIRDYGKQMAGDDCVASRQDEDEEVGFPVKPQVPLRPMTFKGALDLSHFLRE |
| | | | KGGLEG |
| 350 | Fusion/ | Gag, | LKHLVWASRELERFALNPGLLETVSQNFPIVQNLSPRTLNAWVKVIEEKAFSPEVIPMFTALSEGATPHDLNTMLNTIGGHQ |
| | none | Pol, | AAMQMLKDTINEEAAEWDRVHPVHAGPVAPGQMRDPRGSDIAGSTSTLQEQIAWMTNNPPIPVGDIYKRWIIMGLNKIVRMY |
| | | Nef | SPVSILDIKQGPKEPFRDYVDRFYRTLRAEQASQDVKNWMTETLLVQNSNPDCKTILKALGPGATLEEMMSACQGVGGPSHK |
| | | | ARVLAEAMCQLPGKWKPKMIGGIGGFIKVKQYDQGTVLIGPTPVNIIGRNLLTQLGCTLNFPISPIDTVPVKLKPGMDGPRV |
| | | | KQWPLTEEKIKALIEICTEMEKEGKISRIGPENPYNTPIFAIKKKDGTKWRKLVDFRELNKKTQDFWEVQLGIPHPSGLKKK |
| | | | KSVTVLDIGDAYFSVPLDKEFRKYTAFTVPSWGLTTPDKKHQKDPPFLWMGYELHPDRWTVQPIVAKEIVACCDKCQLKGEA |
| | | | IHGQVDCSPGVWQLDCTHLEGKVILVAVHVASGYMEAEVIPTETGQETAYFILKLAGRWPVTTAVKAACWWAGVKQEFGIPY |
| | | | HPQSQGVVESMNNELKKIIGQIRDQAEQLKTAVQMAVLIHNFKRKGGIGEYSAGERIIDIIAITKLQNFRVYYRDNRDPLWK |
| | | | GPARLLWKGEGAVVIQDNSEIKVVPRRKVKIIRDYGKRMAGDDCVAGRQDEDEEVGFPVRPQVPLRPMTYKGALDLSHFLKE |
| | | | KGGLEG |
| 351 | AA/ | Gag, | MGARASVLSGGELDRWEKIRLRPGGKKKYRLKHIVWASRELERFAVNPGLLETAAISPRTLNAWVKVVEEKAFSPEVIPMFS |
| | none | Nef | ALSEGATPQDLNTMLNTVGGHQAAMQMLKETINEEAAEWDRLHPVHAGPIAPGQMREPRGSDIAGTTSTLQEQIGWMTNNPP |
| | | | IPVGEIYKRWIILGLNKIVRMYSPTSILDIRQGPKEPFRDYVDRFYKTLRAEQASQEVKNWMTETLLVQNANPDCKTILKAL |
| | | | GPAATLEEMMTACQGVGGPGHKARVLAEAMSQEEVGFPVKPQVPLRPMTFKGALDLSHFLREKGGLEGTQGFFPDWQNYTPE |
| | | | PGIRFPLTFGWCFKLVPL |
| 430 | AA/ | Gag, | MGARASVLSGGELDRWEKIRLRPGGKKKYRLKHIVWASRELERFAVNPGLLETLKHIVWASRELERFAVNPGLLETAAISPR |
| | none | Nef | TLNAWVKVVEEKAFSPEVIPMFSALSEGATPQDLNTMLNTVGGHQAAMQMLKETINEEAAEWDRLHPVHAGPIAPGQMREPR |
| | | | GSDIAGTTSTLQEQIGWMTNNPPIPVGEIYKRWIILGLNKIVRMYSPTSILDIRQGPKEPERDYVDRFYKTLRAEQASQEVK |
| | | | NWMTETLLVQNANPDCKTILKALGPAATLEEMMTACQGVGGPGHKARVLAEAMSQEEVGFPVKPQVPLRPMTFKGALDLSHF |
| | | | LREKGGLEGTQGFFPDQNYTPEPGIRFPLTFGWCFKLVPL |
| 352 | AA/ | Gag, | LSPRTLNAWVKVIEEKAFSPEVIPMFTALSEGATPHDLNTMLNTIGGHQAAMQMLKDTINEEAAEWDRVHPVHAGPVAPGQM |
| | None | Nef | RDPRGSDIAGSTSTLQEQIAWMTNNPPIPVGDIYKRWIIMGLNKIVRMYSPVSILDIKQGPKEPFRDYVDRFYRTLRAEQAS |
| | | | QDVKNWMTETLLVQNSNPDCKTILKALGPGATLEEMMSACQGVGGPSHKARVLAEAMCQMGARASILSGGKLDKWEKIRLRP |
| | | | GGRKKYKLKHIVWASRELERFAVNPGLLETEEVGFPVRPQVPLRPMTYKGALDLSHFLKEKGGLEGTQGYFPDWQNYTPGPG |
| | | | TRYPLTFGWCFKLVPV |
| 353 | AA/ | Gag, | MWLQSLLLLGTVACSISVMGARASVLSGGELDRWEKIRLRPGGKKKYRLKHIVWASRELERFAVNPGLLETLKHIVWASREL |
| | GM-CSF | Nef | ERFAVNPGLLETAAISPRTLNAWVKVVEEKAFSPEVIPMFSALSEGATPQDLNTMLNTVGGHQAAMQMLKETINEEAAEWDR |
| | | | LHPVHAGPIAPGQMREPRGSDIAGTTSTLQEQIGWMTNNPPIPVGEIYKRWIILGLNKIVRMYSPTSILDIRQGPKEPERDY |
| | | | VDRFYKTLRAEQASQEVKNWMTETLLVQNANPDCKTILKALGPAATLEEMMTACQGVGGPGHKARVLAEAMSQEEVGFPVKP |
| | | | QVPLRPMTFKGALDLSHFLREKGGLEGTQGFFPDQNYTPEPGIRFPLTFGWCFKLVPL |
| 354 | AA/ | Gag, | MDAMKRGLCCVLLLCGAVFVSARMGARASVLSGGELDRWEKIRLRPGGKKKYRLKHIVWASRELERFAVNPGLLETLKHIVW |
| | t-PA | Nef | ASRELERFAVNPGLLETAAISPRTLNAWVKVVEEKAFSPEVIPMFSALSEGATPQDLNTMLNTVGGHQAAMQMLKETINEEA |
| | | | AEWDRLHPVHAGPIAPGQMREPRGSDIAGTTSTLQEQIGWMTNNPPIPVGEIYKRWIILGLNKIVRMYSPTSILDIRQGPKE |
| | | | PFRDYVDRFYKTLRAEQASQEVKNWMTETLLVQNANPDCKTILKALGPAATLEEMMTACQGVGGPGHKARVLAEAMSQEEVG |
| | | | FPVKPQVPLRPMTFKGALDLSHFLREKGGLEGTQGFFPDQNYTPEPGIRFPLTFGWCFKLVPL |
| 355 | AA/ | Gag, | MNPSAAVIFCLILLGLSGTQGILDMAQPVGINTSTTCCYRFINKKIPKQRLESYRRTTSSHCPREAVIFKTKLDKEICADPT |
| | MCP-3 | Nef | QKWVQDFMKHLDKKTQTPKLASAGAMGARASVLSGGELDRWEKIRLRPGGKKKYRLKHIVWASRELERFAVNPGLLETLKHI |
| | | | VWASRELERFAVNPGLLETAAISPRTLNAWVKVVEEKAFSPEVIPMFSALSEGATPQDLNTMLNTVGGHQAAMQMLKETINE |
| | | | EAAEWDRLHPVHAGPIAPGQMREPRGSDIAGTTSTLQEQIGWMTNNPPIPVGEIYKRWIILGLNKIVRMYSPTSILDIRQGP |
| | | | KEPFRDYVDRFYKTLRAEQASQEVKNWMTETLLVQNANPDCKTILKALGPAATLEEMMTACQGVGGPGHKARVLAEAMSQEE |
| | | | VGFPVKPQVPLRPMTFKGALDLSHFLREKGGLEGTQGFFPDQNYTPEPGIRFPLTFGWCFKLVPL |
| 356 | AA/β- | Gag, | MRKAAVSHWQQQSYLDSGIHSGATTTAPSLSMGARASVLSGGELDRWEKIRLRPGGKKKYRLKHIVWASRELERFAVNPGLL |
| | catenin | Nef | ETLKHIVWASRELERFAVNPGLLETAAISPRTLNAWVKVVEEKAFSPEVIPMFSALSEGATPQDLNTMLNTVGGHQAAMQML |
| | | | KETINEEAAEWDRLHPVHAGPIAPGQMREPRGSDIAGTTSTLQEQIGWMTNNPPIPVGEIYKRWIILGLNKIVRMYSPTSIL |
| | | | DIRQGPKEPFRDYVDRFYKTLRAEQASQEVKNWMTETLLVQNANPDCKTILKALGPAATLEEMMTACQGVGGPGHKARVLAE |
| | | | AMSQEEVGFPVKPQVPLRPMTFKGALDLSHFLREKGGLEGTQGFFPDQNYTPEPGIRFPLTFGWCFKLVPL |
| 357 | AA/ | Pol, | GTVLVGPTPVNIIGRNLLTQIGCTLNFPISPIETVPVKLKPGMDGPKVKQWPLTEEKIKALVEICTEMEKEGKISKIGPENP |
| | none | Env | YNTPVFAIKKKDSTKWRKLVDFRELNKRTQDFWEVQLGIPHPAGLKKKKSVTVLDVGDAYFSVPLDKDFRKYTAFTIPSINN |
| | | | ETPGIRYQYNVLPQGWKGSPAIFQSSMTTVKAACWWAGIKQEFGIPYNPQSQGVVESMNKELKKIIGQVRDQAEHLKTAVQM |
| | | | AVFIHNFKRKGGIGGYSAGERIVDIINVSTVQCTHGIRPVVSTQLLLNGSLAEEKRRVVQREKRAVGIGAMFLGFLGAAGST |
| | | | MGAASITLTVQARQLLSGIVQQQNNLLRAIEAQQHLLQLTVWGIKQLQARVLAVERYLKDQQLLGIWGCSGKLICTTVAKEI |
| | | | VASCDKCQLKGEAMHGQVDCSPGIWQLDCTHLEGKIILVAVHVASGYIEAEVIPAETGQETAYFLLKLAGRWPVKTLWVTVY |
| | | | YGVPVWKEAAFPQITLWQRPLVTIKIGGQLKEALLDTGADDTVLEEMNLPGRWKPKMIGGIGGFIKVRQYDQAAAAHNVWAT |
| | | | HACVPTDPNPQEAITKIQNFRVYYRDSRDPLWKGPAKLLWKGEGAVVIQDNSDIKVVPRRKAKIIRDYGKQMAGDDCVASRQ |
| | | | DEDPKFKLPIQKETWETWWTEYWQATWIPEWEFVNTPPLVKLWYQLEKEPIVGAETFYVDGAANRETKAAKEKVYLAWVPAH |
| | | | KGIGGNEQVDKLVSWGFTTPDKKHQKEPPFLWMGYELHPDKWTVQPIVLPEKDSWTVNDIQKLVGKLNWASQIYPGIKVIVI |
| | | | YQYMDDLYVGSDLEIGQHRMRDNWRSELYKYKVV |
| 358 | AA/ | Pol, | AVKAACWWAGVKQEFGIPYHPQSQGVVESMNNELKKIIGQIRDQAEQLKTAVQMAVLIHNFKRKGGIGEYSAGERIIDIIRR |
| | none | Env | RVVQREKRAIGLGAVFLGFLGTAGSTMGAASMTLTVQARLLLSGIVQQQSNLLRAIEAQQHMLQLTVWGIKQLQARILAVER |
| | | | YLRDQQLLGIWGCSGRLICTTVAKEIVACCDKCQLKGEAIHGQVDCSPGVWQLDCTHLEGKVILVAVHVASGYMEAEVIPTE |
| | | | TGQETAYFILKLAGRWPVTTNISTVQCTHGIKPVVSTQLLLNGSLAEKWGLTTPDKKHQKDPPFLWMGYELHPDRWTVQPIE |
| | | | LPEKESWTVNDIQKLIGKLNWASQIYAGIKVIVIYQYVDDLYVGSDLEIEQHRPKFRLPIQKETWDTWWTDYWQATWIPEWE |
| | | | FTNTPPLVKLWYQLETEPIAGVETFYVDGASNRETKLPQITLWQRPIVTIKIGGQIKEALLDTGADDTVLEDMNLPGKWKPK |
| | | | MIGGIGGFIKVKQYDQAALWVTIYYGVPVWKDVHNIWATHACVPTDPSPQEAITKLQNFRVYYRDNRDPLWKGPARLLWKGE |
| | | | GAVVIQDNSEIKVVPRRKVKIIRDYGKRMAGDDCVAGRQDEDGTVLIGPTPVNIIGRNLLTQLGCTLNFPISPIDTVPVKLK |
| | | | PGMDGPRVKQWPLTEEKIKALIEICTEMEKEGKISRIGPENPYNTPIFAIKKKDGTKWRKLVDFRELNKKTQDFWEVQLGIP |
| | | | HPSGLKKKKSVTVLDIGDAYFSVPLDKEFRKYTAFTVPSTNNETPGVRYQYNVLPMGWKGSPAIFQCSMTKEKIYLAWVPAH |
| | | | KGIGGNEQIDKLVSMKDNWRSELYRYKVV |
| 359 | AA/ | Pol, | GTVLVGPTPVNIIGRNLLTQIGCTLNFPISPIETVPVKLKPGMDGPKVKQWPLTEEKIKALVEICTEMEKEGKISKIGPENP |
| | none | Env | YNTPVFAIKKKDSTKWRKLVDFRELNKRTQDFWEVQLGIPHPAGLKKKKSVTVLDVGDAYFSVPLDKDFRKYTAFTIPSINN |
| | | | ETPGIRYQYNVLPQGWKGSPAIFQSSMTTVKAACWWAGIKQEFGIPYNPQSQGVVESMNKELKKIIGQVRDQAEHLKTAVQM |
| | | | AVFIHNFKRKGGIGGYSAGERIVDIINVSTVQCTHGIRPVVSTQLLLNGSLAEEKRRVVQREKRAVGIGAMFLGFLGAAGST |
| | | | MGAASITLTVQARQLLSGIVQQQNNLLRAIEAQQHLLQLTVWGIKQLQARVLAVERYLKDQQLLGIWGCSGKLICTTVAKEI |
| | | | VASCDKCQLKGEAMHGQVDCSPGIWQLDCTHLEGKIILVAVHVASGYIEAEVIPAETGQETAYFLLKLAGRWPVKT |
| 360 | AA/ | Pol, | LWVTVYYGVPVWKEAAFPQITLWQRPLVTIKIGGQLKEALLDTGADDTVLEEMNLPGRWKPKMIGGIGGFIKVRQYDQAAAA |
| | none | Env | HNVWATHACVPTDPNPQEAITKIQNFRVYYRDSRDPLWKGPAKLLWKGEGAVVIQDNSDIKVVPRRKAKIIRDYGKQMAGDD |
| | | | CVASRQDEDPKFKLPIQKETWETWWTEYWQATWIPEWEFVNTPPLVKLWYQLEKEPIVGAETFYVDGAANRETKAAKEKVYL |
| | | | AWVPAHKGIGGNEQVDKLVSWGFTTPDKKHQKEPPFLWMGYELHPDKWTVQPIVLPEKDSWTVNDIQKLVGKLNWASQIYPG |
| | | | IKVIVIYQYMDDLYVGSDLEIGQHRMRDNWRSELYKYKVV |
| 361 | AA/ | Pol, | AVKAACWWAGVKQEFGIPYHPQSQGVVESMNNELKKIIGQIRDQAEQLKTAVQMAVLIHNFKRKGGIGEYSAGERIIDIIRR |
| | none | Env | RVVQREKRAIGLGAVFLGFLGTAGSTMGAASMILTVQARLLLSGIVQQQSNLLRAIEAQQHMLQLTVWGIKQLQARILAVER |
| | | | YLRDQQLLGIWGCSGRLICTTVAKEIVACCDKCQLKGEAIHGQVDCSPGVWQLDCTHLEGKVILVAVHVASGYMEAEVIPTE |
| | | | TGQETAYFILKLAGRWPVTTNISTVQCTHGIKPVVSTQLLLNGSLAEKWGLTTPDKKHQKDPPFLWMGYELHPDRWTVQPIE |
| | | | LPEKESWTVNDIQKLIGKLNWASQIYAGIKVIVIYQYVDDLYVGSDLEIEQHRPKFRLPIQKETWDTWWIDYWQATWIPEWE |
| | | | FTNTPPLVKLWYQLETEPIAGVETFYVDGASNRETKLPQITLWQRPIVTIKIGGQIKEALLDTGADDTVLEDMNLPGKWKPK |
| | | | MIGGIGGFIKVKQYDQAA |
| 362 | AA/ | Pol, | LWVTIYYGVPVWKDVHNIWATHACVPTDPSPQEAITKLQNFRVYYRDNRDPLWKGPARLLWKGEGAVVIQDNSEIKVVPRRK |
| | none | Env | VKIIRDYGKRMAGDDCVAGRQDEDGTVLIGPTPVNIIGRNLLTQLGCTLNFPISPIDTVPVKLKPGMDGPRVKQWPLTEEKI |
| | | | KALIEICTEMEKEGKISRIGPENPYNTPIFAIKKKDGTKWRKLVDFRELNKKTQDFWEVQLGIPHPSGLKKKKSVTVLDIGD |
| | | | AYFSVPLDKEFRKYTAFTVPSTNNETPGVRYQYNVLPMGWKGSPAIFQCSMTKEKIYLAWVPAHKGIGGNEQIDKLVSMKDN |
| | | | WRSELYRYKVV |
| 363 | AA/ | Pol, | MWLQSLLLLGTVACSISVGTVLVGPTPVNIIGRNLLTQIGCTLNFPISPIETVPVKLKPGMDGPKVKQWPLTEEKIKALVEI |
| | GM-CSF | Env | CTEMEKEGKISKIGPENPYNTPVFAIKKKDSTKWRKLVDFRELNKRTQDFWEVQLGIPHPAGLKKKKSVTVLDVGDAYFSVP |
| | | | LDKDFRKYTAFTIPSINNETPGIRYQYNVLPQGWKGSPAIFQSSMTTVKAACWWAGIKQEFGIPYNPQSQGVVESMNKELKK |
| | | | IIGQVRDQAEHLKTAVQMAVFIHNFKRKGGIGGYSAGERIVDIINVSTVQCTHGIRPVVSTQLLLNGSLAEEKRRVVQREKR |
| | | | AVGIGAMFLGFLGAAGSTMGAASITLTVQARQLLSGIVQQQNNLLRAIEAQQHLLQLTVWGIKQLQARVLAVERYLKDQQLL |
| | | | GIWGCSGKLICTTVAKEIVASCDKCQLKGEAMHGQVDCSPGIWQLDCTHLEGKIILVAVHVASGYIEAEVIPAETGQETAYF |
| | | | LLKLAGRWPVKTLWVTVYYGVPVWKEAAFPQITLWQRPLVTIKIGGQLKEALLDTGADDTVLEEMNLPGRWKPKMIGGIGGF |
| | | | IKVRQYDQAAAAHNVWATHACVPTDPNPQEAITKIQNFRVYYRDSRDPLWKGPAKLLWKGEGAVVIQDNSDIKVVPRRKAKI |
| | | | IRDYGKQMAGDDCVASRQDEDPKFKLPIQKETWETWWTEYWQATWIPEWEEVNTPPLVKLWYQLEKEPIVGAETFYVDGAAN |
| | | | RETKAAKEKVYLAWVPAHKGIGGNEQVDKLVSWGFTTPDKKHQKEPPFLWMGYELHPDKWTVQPIVLPEKDSWTVNDIQKLV |
| | | | GKLNWASQIYPGIKVIVIYQYMDDLYVGSDLEIGQHRMRDNWRSELYKYKVV |
| 364 | AA/ | Pol, | MDAMKRGLCCVLLLCGAVFVSARGTVLVGPTPVNIIGRNLLTQIGCTLNFPISPIETVPVKLKPGMDGPKVKQWPLTEEKIK |
| | t-PA | Env | ALVEICTEMEKEGKISKIGPENPYNTPVFAIKKKDSTKWRKLVDFRELNKRTQDFWEVQLGIPHPAGLKKKKSVIVLDVGDA |
| | | | YFSVPLDKDFRKYTAFTIPSINNETPGIRYQYNVLPQGWKGSPAIFQSSMTTVKAACWWAGIKQEFGIPYNPQSQGVVESMN |
| | | | KELKKIIGQVRDQAEHLKTAVQMAVFIHNFKRKGGIGGYSAGERIVDIINVSTVQCTHGIRPVVSTQLLLNGSLAEEKRRVV |
| | | | QREKRAVGIGAMFLGFLGAAGSTMGAASITLTVQARQLLSGIVQQQNNLLRAIEAQQHLLQLTVWGIKQLQARVLAVERYLK |
| | | | DQQLLGIWGCSGKLICTTVAKEIVASCDKCQLKGEAMHGQVDCSPGIWQLDCTHLEGKIILVAVHVASGYIEAEVIPAETGQ |
| | | | ETAYFLLKLAGRWPVKTLWVTVYYGVPVWKEAAFPQITLWQRPLVTIKIGGQLKEALLDTGADDTVLEEMNLPGRWKPKMIG |
| | | | GIGGFIKVRQYDQAAAAHNVWATHACVPTDPNPQEAITKIQNFRVYYRDSRDPLWKGPAKLLWKGEGAVVIQDNSDIKVVPR |
| | | | RKAKIIRDYGKQMAGDDCVASRQDEDPKFKLPIQKETWETWWTEYWQATWIPEWEFVNTPPLVKLWYQLEKEPIVGAETFYV |
| | | | DGAANRETKAAKEKVYLAWVPAHKGIGGNEQVDKLVSWGFTTPDKKHQKEPPFLWMGYELHPDKWTVQPIVLPEKDSWTVND |
| | | | IQKLVGKLNWASQIYPGIKVIVIYQYMDDLYVGSDLEIGQHRMRDNWRSELYKYKVV |
| 365 | AA/ | Pol, | MNPSAAVIFCLILLGLSGTQGILDMAQPVGINTSTTCCYRFINKKIPKQRLESYRRTTSSHCPREAVIFKTKLDKEICADPT |
| | MCP-3 | Env | QKWVQDFMKHLDKKTQTPKLASAGAGTVLVGPTPVNIIGRNLLTQIGCTLNFPISPIETVPVKLKPGMDGPKVKQWPLTEEK |
| | | | IKALVEICTEMEKEGKISKIGPENPYNTPVFAIKKKDSTKWRKLVDFRELNKRTQDFWEVQLGIPHPAGLKKKKSVTVLDVG |
| | | | DAYFSVPLDKDFRKYTAFTIPSINNETPGIRYQYNVLPQGWKGSPAIFQSSMTTVKAACWWAGIKQEFGIPYNPQSQGVVES |
| | | | MNKELKKIIGQVRDQAEHLKTAVQMAVFIHNFKRKGGIGGYSAGERIVDIINVSTVQCTHGIRPVVSTQLLLNGSLAEEKRR |
| | | | VVQREKRAVGIGAMFLGELGAAGSTMGAASITLTVQARQLLSGIVQQQNNLLRAIEAQQHLLQLTVWGIKQLQARVLAVERY |
| | | | LKDQQLLGIWGCSGKLICTTVAKEIVASCDKCQLKGEAMHGQVDCSPGIWQLDCTHLEGKIILVAVHVASGYIEAEVIPAET |
| | | | GQETAYFLLKLAGRWPVKTLWVTVYYGVPVWKEAAFPQITLWQRPLVTIKIGGQLKEALLDTGADDTVLEEMNLPGRWKPKM |
| | | | IGGIGGFIKVRQYDQAAAAHNVWATHACVPTDPNPQEAITKIQNFRVYYRDSRDPLWKGPAKLLWKGEGAVVIQDNSDIKVV |
| | | | PRRKAKIIRDYGKQMAGDDCVASRQDEDPKFKLPIQKETWETWWTEYWQATWIPEWEFVNTPPLVKLWYQLEKEPIVGAETF |
| | | | YVDGAANRETKAAKEKVYLAWVPAHKGIGGNEQVDKLVSWGFTTPDKKHQKEPPFLWMGYELHPDKWTVQPIVLPEKDSWTV |
| | | | NDIQKLVGKLNWASQIYPGIKVIVIYQYMDDLYVGSDLEIGQHRMRDNWRSELYKYKVV |
| 366 | AA/β- | Pol, | MRKAAVSHWQQQSYLDSGIHSGATTTAPSLSGTVLVGPTPVNIIGRNLLTQIGCTLNFPISPIETVPVKLKPGMDGPKVKQW |
| | catenin | Env | PLTEEKIKALVEICTEMEKEGKISKIGPENPYNTPVFAIKKKDSTKWRKLVDFRELNKRTQDFWEVQLGIPHPAGLKKKKSV |
| | | | TVLDVGDAYFSVPLDKDFRKYTAFTIPSINNETPGIRYQYNVLPQGWKGSPAIFQSSMTTVKAACWWAGIKQEFGIPYNPQS |
| | | | QGVVESMNKELKKIIGQVRDQAEHLKTAVQMAVFIHNFKRKGGIGGYSAGERIVDIINVSTVQCTHGIRPVVSTQLLLNGSL |
| | | | AEEKRRVVQREKRAVGIGAMFLGELGAAGSTMGAASITLTVQARQLLSGIVQQQNNLLRAIEAQQHLLQLTVWGIKQLQARV |
| | | | LAVERYLKDQQLLGIWGCSGKLICTTVAKEIVASCDKCQLKGEAMHGQVDCSPGIWQLDCTHLEGKIILVAVHVASGYIEAE |
| | | | VIPAETGQETAYFLLKLAGRWPVKTLWVTVYYGVPVWKEAAFPQITLWQRPLVTIKIGGQLKEALLDTGADDTVLEEMNLPG |
| | | | RWKPKMIGGIGGFIKVRQYDQAAAAHNVWATHACVPTDPNPQEAITKIQNFRVYYRDSRDPLWKGPAKLLWKGEGAVVIQDN |
| | | | SDIKVVPRRKAKIIRDYGKQMAGDDCVASRQDEDPKFKLPIQKETWETWWTEYWQATWIPEWEFVNTPPLVKLWYQLEKEPI |
| | | | VGAETFYVDGAANRETKAAKEKVYLAWVPAHKGIGGNEQVDKLVSWGFTTPDKKHQKEPPFLWMGYELHPDKWTVQPIVLPE |
| | | | KDSWTVNDIQKLVGKLNWASQIYPGIKVIVIYQYMDDLYVGSDLEIGQHRMRDNWRSELYKYKVV |
| 407 | AA/ | Pol | TVKAACWWAGIKQEFGIPYNPQSQGVVESMNKELKKIIGQVRDQAEHLKTAVQMAVFIHNFKRKGGIGGYSAGERIVDIIAI |
| | none | | TKIQNFRVYYRDSRDPLWKGPAKLLWKGEGAVVIQDNSDIKVVPRRKAKIIRDYGKQMAGDDCVASRQDEDPKFKLPIQKET |
| | | | WETWWTEYWQATWIPEWEFVNTPPLVKLWYQLEKEPIVGAETFYVDGAANRETKAAKEKVYLAWVPAHKGIGGNEQVDKLVS |
| | | | WGFTTPDKKHQKEPPFLWMGYELHPDKWTVQPIVLPEKDSWTVNDIQKLVGKLNWASQIYPGIKVIVIYQYMDDLYVGSDLE |
| | | | IGQHRVAKEIVASCDKCQLKGEAMHGQVDCSPGIWQLDCTHLEGKIILVAVHVASGYIEAEVIPAETGQETAYFLLKLAGRW |
| | | | PVKTAAFPQITLWQRPLVTIKIGGQLKEALLDTGADDTVLEEMNLPGRWKPKMIGGIGGFIKVRQYDQGTVLVGPTPVNIIG |
| | | | RNLLTQIGCTLNFPISPIETVPVKLKPGMDGPKVKQWPLTEEKIKALVEICTEMEKEGKISKIGPENPYNTPVFAIKKKDST |
| | | | KWRKLVDFRELNKRTQDFWEVQLGIPHPAGLKKKKSVTVLDVGDAYFSVPLDKDFRKYTAFTIPSINNETPGIRYQYNVLPQ |
| | | | GWKGSPAIFQSSMT |
| 408 | AA, | Pol | AVKAACWWAGVKQEFGIPYHPQSQGVVESMNNELKKIIGQIRDQAEQLKTAVQMAVLIHNFKRKGGIGEYSAGERIIDIIVA |
| | AAY/ | | KEIVACCDKCQLKGEAIHGQVDCSPGVWQLDCTHLEGKVILVAVHVASGYMEAEVIPTETGQETAYFILKLAGRWPVTTWGL |
| | none | | TTPDKKHQKDPPFLWMGYELHPDRWTVQPIELPEKESWTVNDIQKLIGKLNWASQIYAGIKVIVIYQYVDDLYVGSDLEIEQ |
| | | | HRLPQITLWQRPIVTIKIGGQIKEALLDTGADDTVLEDMNLPGKWKPKMIGGIGGFIKVKQYDQPKFRLPIQKETWDTWWTD |
| | | | YWQATWIPEWEFTNTPPLVKLWYQLETEPIAGVETFYVDGASNRETKAAYAITKLQNFRVYYRDNRDPLWKGPARLLWKGEG |
| | | | AVVIQDNSEIKVVPRRKVKIIRDYGKRMAGDDCVAGRQDEDGTVLIGPTPVNIIGRNLLTQLGCTLNFPISPIDTVPVKLKP |
| | | | GMDGPRVKQWPLTEEKIKALIEICTEMEKEGKISRIGPENPYNTPIFAIKKKDGTKWRKLVDFRELNKKTQDFWEVQLGIPH |
| | | | PSGLKKKKSVTVLDIGDAYFSVPLDKEFRKYTAFTVPSTNNETPGVRYQYNVLPMGWKGSPAIFQCSMTKEKIYLAWVPAHK |
| | | | GIGGNEQIDKLVS |
| 409 | AA/ | Pol/ | TVKAACWWAGIKQEFGIPYNPQSQGVVESMNKELKKIIGQVRDQAEHLKTAVQMAVFIHNFKRKGGIGGYSAGERIVDIIVA |
| | none | no | KEIVASCDKCQLKGEAMHGQVDCSPGIWQLDCTHLEGKIILVAVHVASGYIEAEVIPAETGQETAYFLLKLAGRWPVKTAIT |
| | | YMDD | KIQNFRVYYRDSRDPLWKGPAKLLWKGEGAVVIQDNSDIKVVPRRKAKIIRDYGKQMAGDDCVASRQDEDPKFKLPIQKETW |
| | | | ETWWTEYWQATWIPEWEFVNTPPLVKLWYQLEKEPIVGAETFYVDGAANRETKAAKEKVYLAWVPAHKGIGGNEQVDKLVSW |
| | | | GFTTPDKKHQKEPPFLWMGYELHPDKWTVQPIVLPEKDSWTVNDIQKLVGKLNWASQIYPGIKVAAFPQITLWQRPLVTIKI |
| | | | GGQLKEALLDTGADDTVLEEMNLPGRWKPKMIGGIGGFIKVRQYDQGTVLVGPTPVNIIGRNLLTQIGCTLNFPISPIETVP |
| | | | VKLKPGMDGPKVKQWPLTEEKIKALVEICTEMEKEGKISKIGPENPYNTPVFAIKKKDSTKWRKLVDFRELNKRTQDFWEVQ |
| | | | LGIPHPAGLKKKKSVTVLDVGDAYFSVPLDKDFRKYTAFTIPSINNETPGIRYQYNVLPQGWKGSPAIFQSSMT |
| 410 | AA, | Pol | AVKAACWWAGVKQEFGIPYHPQSQGVVESMNNELKKIIGQIRDQAEQLKTAVQMAVLIHNFKRKGGIGEYSAGERIIDIIWG |
| | AAY/ | | LTTPDKKHQKDPPFLWMGYELHPDRWTVQPIELPEKESWTVNDIQKLIGKLNWASQIYAGIKVAAYVAKEIVACCDKCQLKG |
| | none | | EAIHGQVDCSPGVWQLDCTHLEGKVILVAVHVASGYMEAEVIPTETGQETAYFILKLAGRWPVTTLPQITLWQRPIVTIKIG |
| | | | GQIKEALLDTGADDTVLEDMNLPGKWKPKMIGGIGGFIKVKQYDQPKERLPIQKETWDTWWIDYWQATWIPEWEFTNTPPLV |
| | | | KLWYQLETEPIAGVETFYVDGASNRETKAAYAITKLQNFRVYYRDNRDPLWKGPARLLWKGEGAVVIQDNSEIKVVPRRKVK |
| | | | IIRDYGKRMAGDDCVAGRQDEDGTVLIGPTPVNIIGRNLLTQLGCTLNFPISPIDTVPVKLKPGMDGPRVKQWPLTEEKIKA |
| | | | LIEICTEMEKEGKISRIGPENPYNTPIFAIKKKDGTKWRKLVDFRELNKKTQDFWEVQLGIPHPSGLKKKKSVTVLDIGDAY |
| | | | FSVPLDKEFRKYTAFTVPSTNNETPGVRYQYNVLPMGWKGSPAIFQCSMTKEKIYLAWVPAHKGIGGNEQIDKLVS |
| 367 | AA, | Gag, | YQYNVLPQGASRELERFAVNPGLLWIILGLNKIVRMYSPTSIAARTLNAWVKVFLWMGYELHLTFGWCFKLPLWKGPAKLVT |
| | AAA/ | Pol, | VYYGVPVAALLWKGEGAVAAAKLVGKLNWAKLLWKGEGATLNFPISPIWQATWIPEWKAACWWAGIRQANFLGKIWPSHKGR |
| | none | Nef, | NVWATHACVAAEMMTACQGVSTVQCTHGIAAKQMAGDDCVAAWQLDCTHLEYKAAVDLSHFLREKGGLEGAAYYMDDLYVGS |
| | | Env | GQVDCSPGIATLEEMMTAELHPDKWTVWTVNDIQKLGIWGCSGKLTVNDIQKLVIVTDSQYALYVDRFYKTLRAEQASQEVD |
| | | | LNTMLNTVKLTPLCVTLYQYMDDLYVVIYQYMDDL |
| 431 | AA, | Gag, | YQYNVLPQGASRELERFAVNPGLLWIILGLNKIVRMYSPTSIAARTLNAWVKVFLWMGYELHLTFGWCFKLPLWKGPAKLVT |
| | AAA/ | Pol, | VYYGVPVAALLWKGEGAVAAAKLVGKLNWAKLLWKGEGATLNFPISPIWQATWIPEWKAACWWAGIRQANFLGKIWPSHKGR |
| | none | Nef, | NVWATHACVAAEMMTACQGVSTVQCTHGIAAKQMAGDDCVAAWQLDCTHLEYKAAVDLSHFLREKGGLEGAAYYMDDLYVGS |
| | | Env | GQVDCSPGIATLEEMMTAELHPDKWTVWTVNDIQKLGIWGCSGKLTVNDIQKLVIVTDSQYALYVDRFYKTLYVDRFYKTLR |
| | | | AEQASQEVDLNTMLNTVKLTPLCVTLYQYMDDLYVVIYQYMDDLWIILGLNKI |
| 368 | AA, | Env, | MWLQSLLLLGTVACSISVYQYNVLPQGASRELERFAVNPGLLWIILGLNKIVRMYSPTSIAARTLNAWVKVFLWMGYELHLT |
| | AAA/ | Gag, | FGWCFKLPLWKGPAKLVTVYYGVPVAALLWKGEGAVAAAKLVGKLNWAKLLWKGEGATLNFPISPIWQATWIPEWKAACWWA |
| | GM-CSF | Nef, | GIRQANFLGKIWPSHKGRNVWATHACVAAEMMTACQGVSTVQCTHGIAAKQMAGDDCVAAWQLDCTHLEYKAAVDLSHFLRE |
| | | Pol | KGGLEGAAYYMDDLYVGSGQVDCSPGIATLEEMMTAELHPDKWTVWTVNDIQKLGIWGCSGKLTVNDIQKLVIVTDSQYALY |
| | | | VDRFYKTLRAEQASQEVDLNTMLNTVKLTPLCVTLYQYMDDLYVVIYQYMDDL |
| 432 | AA, | Env, | MWLQSLLLLGTVACSISVYQYNVLPQGASRELERFAVNPGLLWIILGLNKIVRMYSPTSIAARTLNAWVKVFLWMGYELHLT |
| | AAA/ | Gag, | FGWCFKLPLWKGPAKLVTVYYGVPVAALLWKGEGAVAAAKLVGKLNWAKLLWKGEGATLNFPISPIWQATWIPEWKAACWWA |
| | GM-CSF | Nef, | GIRQANFLGKIWPSHKGRNVWATHACVAAEMMTACQGVSTVQCTHGIAAKQMAGDDCVAAWQLDCTHLEYKAAVDLSHFLRE |
| | | Pol | KGGLEGAAYYMDDLYVGSGQVDCSPGIATLEEMMTAELHPDKWTVWTVNDIQKLGIWGCSGKLTVNDIQKLVIVTDSQYALY |
| | | | VDRFYKTLYVDRFYKTLRAEQASQEVDLNTMLNTVKLTPLCVTLYQYMDDLYVVIYQYMDDLWIILGLNKI |
| 369 | AA, | Env, | MDAMKRGLCCVLLLCGAVFVSARYQYNVLPQGASRELERFAVNPGLLWIILGLNKIVRMYSPTSIAARTLNAWVKVFLWMGY |
| | AAA/ | Gag, | ELHLTFGWCFKLPLWKGPAKLVTVYYGVPVAALLWKGEGAVAAAKLVGKLNWAKLLWKGEGATLNFPISPIWQATWIPEWKA |
| | t-PA | Nef, | ACWWAGIRQANFLGKIWPSHKGRNVWATHACVAAEMMTACQGVSTVQCTHGIAAKQMAGDDCVAAWQLDCTHLEYKAAVDLS |
| | | Pol | HFLREKGGLEGAAYYMDDLYVGSGQVDCSPGIATLEEMMTAELHPDKWTVWTVNDIQKLGIWGCSGKLTVNDIQKLVIVTDS |
| | | | QYALYVDRFYKTLRAEQASQEVDLNTMLNTVKLTPLCVTLYQYMDDLYVVIYQYMDDL |
| 433 | AA, | Env, | MDAMKRGLCCVLLLCGAVFVSARYQYNVLPQGASRELERFAVNPGLLWIILGLNKIVRMYSPTSIAARTLNAWVKVFLWMGY |
| | AAA/ | Gag, | ELHLTFGWCFKLPLWKGPAKLVTVYYGVPVAALLWKGEGAVAAAKLVGKLNWAKLLWKGEGATLNFPISPIWQATWIPEWKA |
| | t-PA | Nef, | ACWWAGIRQANFLGKIWPSHKGRNVWATHACVAAEMMTACQGVSTVQCTHGIAAKQMAGDDCVAAWQLDCTHLEYKAAVDLS |
| | | Pol | HFLREKGGLEGAAYYMDDLYVGSGQVDCSPGIATLEEMMTAELHPDKWTVWTVNDIQKLGIWGCSGKLTVNDIQKLVIVTDS |
| | | | QYALYVDRFYKTLYVDRFYKTLRAEQASQEVDLNTMLNTVKLTPLCVTLYQYMDDLYVVIYQYMDDLWIILGLNKI |
| 370 | AA, | Env, | MNPSAAVIFCLILLGLSGTQGILDMAQPVGINTSTTCCYRFINKKIPKQRLESYRRTTSSHCPREAVIFKTKLDKEICADPT |
| | AAA/ | Gag, | QKWVQDFMKHLDKKTQTPKLASAGAYQYNVLPQGASRELERFAVNPGLLWIILGLNKIVRMYSPTSIAARTLNAWVKVFLWM |
| | MCP-3 | Nef, | GYELHLTFGWCFKLPLWKGPAKLVTVYYGVPVAALLWKGEGAVAAAKLVGKLNWAKLLWKGEGATLNFPISPIWQATWIPEW |
| | | Pol | KAACWWAGIRQANFLGKIWPSHKGRNVWATHACVAAEMMTACQGVSTVQCTHGIAAKQMAGDDCVAAWQLDCTHLEYKAAVD |
| | | | LSHFLREKGGLEGAAYYMDDLYVGSGQVDCSPGIATLEEMMTAELHPDKWTVWTVNDIQKLGIWGCSGKLTVNDIQKLVIVT |
| | | | DSQYALYVDRFYKTLRAEQASQEVDLNTMLNTVKLTPLCVTLYQYMDDLYVVIYQYMDDL |
| 434 | AA, | Env, | MNPSAAVIFCLILLGLSGTQGILDMAQPVGINTSTTCCYRFINKKIPKQRLESYRRTTSSHCPREAVIFKTKLDKEICADPT |
| | AAA/ | Gag, | QKWVQDFMKHLDKKTQTPKLASAGAYQYNVLPQGASRELERFAVNPGLLWIILGLNKIVRMYSPTSIAARTLNAWVKVFLWM |
| | MCP-3 | Nef, | GYELHLTFGWCFKLPLWKGPAKLVTVYYGVPVAALLWKGEGAVAAAKLVGKLNWAKLLWKGEGATLNFPISPIWQATWIPEW |
| | | Pol | KAACWWAGIRQANFLGKIWPSHKGRNVWATHACVAAEMMTACQGVSTVQCTHGIAAKQMAGDDCVAAWQLDCTHLEYKAAVD |
| | | | LSHFLREKGGLEGAAYYMDDLYVGSGQVDCSPGIATLEEMMTAELHPDKWTVWTVNDIQKLGIWGCSGKLTVNDIQKLVIVT |
| | | | DSQYALYVDRFYKTLYVDRFYKTLRAEQASQEVDLNTMLNTVKLTPLCVTLYQYMDDLYVVIYQYMDDLWIILGLNKI |
| 371 | AA, | Env, | MRKAAVSHWQQQSYLDSGIHSGATTTAPSLSYQYNVLPQGASRELERFAVNPGLLWIILGLNKIVRMYSPTSIAARTLNAWV |
| | AAA/ | Gag, | KVFLWMGYELHLTFGWCFKLPLWKGPAKLVTVYYGVPVAALLWKGEGAVAAAKLVGKLNWAKLLWKGEGATLNFPISPIWQA |
| | β- | Nef, | TWIPEWKAACWWAGIRQANFLGKIWPSHKGRNVWATHACVAAEMMTACQGVSTVQCTHGIAAKQMAGDDCVAAWQLDCTHLE |
| | catenin | Pol | YKAAVDLSHFLREKGGLEGAAYYMDDLYVGSGQVDCSPGIATLEEMMTAELHPDKWTVWTVNDIQKLGIWGCSGKLTVNDIQ |
| | | | KLVIVTDSQYALYVDRFYKTLRAEQASQEVDLNTMLNTVKLTPLCVTLYQYMDDLYVVIYQYMDDL |
| 435 | AA, | Env, | MRKAAVSHWQQQSYLDSGIHSGATTTAPSLSYQYNVLPQGASRELERFAVNPGLLWIILGLNKIVRMYSPTSIAARTLNAWV |
| | AAA/ | Gag, | KVFLWMGYELHLTFGWCFKLPLWKGPAKLVTVYYGVPVAALLWKGEGAVAAAKLVGKLNWAKLLWKGEGATLNFPISPIWQA |
| | p- | Nef, | TWIPEWKAACWWAGIRQANFLGKIWPSHKGRNVWATHACVAAEMMTACQGVSTVQCTHGIAAKQMAGDDCVAAWQLDCTHLE |
| | catenin | Pol | YKAAVDLSHFLREKGGLEGAAYYMDDLYVGSGQVDCSPGIATLEEMMTAELHPDKWTVWTVNDIQKLGIWGCSGKLTVNDIQ |
| | | | KLVIVTDSQYALYVDRFYKTLYVDRFYKTLRAEQASQEVDLNTMLNTVKLTPLCVTLYQYMDDLYVVIYQYMDDLWIILGLN |
| | | | KI |
| 424 | REKR/ | Env, | IRTLNAWVKVREKRDLNTMLNTVREKRWIILGLNKIREKRYVDRFYKTLREKRATLEEMMTAREKREMMTACQGVREKRTLN |
| | none | Gag, | FPISPIREKRYQYNVLPQGREKRVIYQYMDDLREKRYQYMDDLYVREKRYMDDLYVGSREKRFLWMGYELHREKRELHPDKW |
| | | Nef, | TVREKRWTVNDIQKLREKRTVNDIQKLVREKRKLVGKLNWAREKRWQATWIPEWREKRIVTDSQYALREKRGQVDCSPGIRE |
| | | Pol | KRWQLDCTHLEREKRKAACWWAGIREKRPLWKGPAKLREKRKLLWKGEGAREKRLLWKGEGAVREKRKQMAGDDCVREKRVT |
| | | | VYYGVPVREKRNVWATHACVREKRKLTPLCVTLREKRSTVQCTHGIREKRGIWGCSGKLREKRLTFGWCFKLREKRASRELE |
| | | | RFAVNPGLLREKRWIILGLNKIVRMYSPTSIREKRYVDRFYKTLRAEQASQEVREKRRQANFLGKIWPSHKGRREKRYKAAV |
| | | | DLSHFLREKGGLEG |
| 372 | REKR/ | Env, | MAPRSARRPLLLLLLLLLLGLMHCASAAMFMVKNGNGTACIMANFSAAFSVNYDTKSGPKNMTLDLPSDATVVLNRSSCGKE |
| | LAMP-1 | Gag, | NTSDPSLVIAFGRGHTLTLNFTRNATRYSVQLMSFVYNLSDTHLFPNASSKEIKTVESITDIRADIDKKYRCVSGTQVHMNN |
| | N- and | Nef, | VTVTLHDATIQAYLSNSSFSRGETRCEQDRPSPTTAPPAPPSPSPSPVPKSPSVDKYNVSGTNGTCLLASMGLQLNLTYERK |
| | C-term | Pol | DNTTVTRLLNINPNKTSASGSCGAHLVTLELHSEGTTVLLFQFGMNASSSRFFLQGIQLNTlLPDARDPAFKAANGSLRALQ |
| | signal | | ATVGNSYKCNAEEHVRVTKAFSVNIFKVWVQAFKVEGGQFGSVEECLLDENSLEDIRTLNAWVKVREKRDLNTMLNTVREKR |
| | seg | | WIILGLNKIREKRYVDRFYKTLREKRATLEEMMTAREKREMMTACQGVREKRTLNFPISPIREKRYQYNVLPQGREKRVIYQ |
| | | | YMDDLREKRYQYMDDLYVREKRYMDDLYVGSREKRFLWMGYELHREKRELHPDKWTVREKRWTVNDIQKLREKRTVNDIQKL |
| | | | VREKRKLVGKLNWAREKRWQATWIPEWREKRIVTDSQYALREKRGQVDCSPGIREKRWQLDCTHLEREKRKAACWWAGIREK |
| | | | RPLWKGPAKLREKRKLLWKGEGAREKRLLWKGEGAVREKRKQMAGDDCVREKRVTVYYGVPVREKRNVWATHACVREKRKLT |
| | | | PLCVTLREKRSTVQCTHGIREKRGIWGCSGKLREKRLTFGWCFKLREKRASRELERFAVNPGLLREKRWIILGLNKIVRMYS |
| | | | PTSIREKRYVDRFYKTLRAEQASQEVREKRRQANFLGKIWPSHKGRREKRYKAAVDLSHFLREKGGLEGGSEFTLIPIAVGG |
| | | | ALAGLVIVLIAYLVGRKRSHAGYQTI |
| 373 | AA, | Env, | ASRELERFAVNPGLLQLKGEAMHGQVDCSPGIWQLDCTHLCSATEKLWVTVYYGVPVWKEATTTLPVGEIYKRWIILGLNKI |
| | AAA/ | Gag, | VRMYSPTSINNETPGIRYQYNVLPQGWKGSPAIFAAPLWKGPAKLLWKGEGAVVIQDNSDIAAIVLPEKDSWTVNDIQKLVG |
| | none | Nef, | KLNWASVYYRDSRDPLWKGPAKLLWKGEGAVSNFTSTTVKAACWWAGIKQEFGIPYVLPEKDSWTVNDIQKLVGKLNWASQM |
| | | Pol | VHQAISPRTLNAWVKVVEEKAFSPAAWETWWTEYWQATWIPEWEFVNTPPLQDSGLEVNIVTDSQYALGIIQAQPDSWTVND |
| | | | IQKLVGKLNWASQIYPGIKNPDIVIYQYMDDLYVGSDLEIGQHRAADPLWKGPAKLLWKGEGAVVIQDNSDRQANFLGKIWP |
| | | | SHKGRAAPPFLWMGYELHPDKWTVQPIVLPEKFRKQNPDIVIYQYMDDLYVGSDLEINLLTQIGCTLNFPISPIETVPVKLK |
| | | | GPKEPFRDYVDRFYKTLRAEQASQEVYLKDQQLLGIWGCSGKLICTTAVPWAAAKKHQKEPPFLWMGYELHPDKWTVQPGTG |
| | | | PCTNVSTVQCTHGIRPVVSTQLKQNPDIVIYQYMDDLYVGSDLEIGQLGPAATLEEMMTACQGVGGPGHKARDQSLKPCVKL |
| | | | TPLCVTLNCTDLRNTGPGIRYPLLTFGWCFKLPVEPEKVEAKIIRDYGKQMAGDDCVASRQDEDILKALGPAATLEEMMTAC |
| | | | QGVGGPGYKAAVDLSHFLREKGGLEGAAYAAKAYDTEVHNVWATHACVPTDPNPQEAAALSEGATPQDLNTMLNTVGGHQAA |
| | | | MQQVDCSPGIWQLDCTHLEGKIILVAV |
| 411 | AA, | Env, | QLKGEAMHGQVDCSPGIWQLDCTHLCSATEKLWVTVYYGVPVWKEATTTLASRELERFAVNPGLLNNETPGIRYQYNVLPQG |
| | AAA/ | Gag, | WKGSPAIFPVGEIYKRWIILGLNKIVRMYSPTSIAAPPFLWMGYELHPDKWTVQPIVLPEKWETWWTEYWQATWIPEWEFVN |
| | none | Nef, | TPPLQDSGLEVNIVTDSQYALGIIQAQPDSWTVNDIQKLVGKLNWASQIYPGIKAADPLWKGPAKLLWKGEGAVVIQDNSDR |
| | | Pol | QANFLGKIWPSHKGRPLWKGPAKLLWKGEGAVVIQDNSDIAASNFTSTTVKAACWWAGIKQEFGIPYVLPEKDSWTVNDIQK |
| | | | LVGKLNWASQMVHQAISPRTLNAWVKVVEEKAFSPIVLPEKDSWTVNDIQKLVGKLNWASGTGPCTNVSTVQCTHGIRPVVS |
| | | | TQLYLKDQQLLGIWGCSGKLICTTAVPWAAAKKHQKEPPFLWMGYELHPDKWTVQPVYYRDSRDPLWKGPAKLLWKGEGAVN |
| | | | LLTQIGCTLNFPISPIETVPVKLKGPKEPFRDYVDRFYKTLRAEQASQEVLGPAATLEEMMTACQGVGGPGHKARDQSLKPC |
| | | | VKLTPLCVTLNCTDLRNTGPGIRYPLLTFGWCFKLPVEPEKVEAKIIRDYGKQMAGDDCVASRQDEDILKALGPAATLEEMM |
| | | | TACQGVGGPGYKAAVDLSHFLREKGGLEGAAYAAKAYDTEVHNVWATHACVPTDPNPQEAAALSEGATPQDLNTMLNTVGGH |
| | | | QAAMQQVDCSPGIWQLDCTHLEGKIILVAV |
| 374 | AA, | Env, | MWLQSLLLLGTVACSISVASRELERFAVNPGLLQLKGEAMHGQVDCSPGIWQLDCTHLCSATEKLWVTVYYGVPVWKEATTT |
| | AAA/ | Gag, | LPVGEIYKRWIILGLNKIVRMYSPTSINNETPGIRYQYNVLPQGWKGSPAIFAAPLWKGPAKLLWKGEGAVVIQDNSDIAAI |
| | GM-CSF | Nef, | VLPEKDSWTVNDIQKLVGKLNWASVYYRDSRDPLWKGPAKLLWKGEGAVSNFTSTTVKAACWWAGIKQEFGIPYVLPEKDSW |
| | | Pol | TVNDIQKLVGKLNWASQMVHQAISPRTLNAWVKVVEEKAFSPAAWETWWTEYWQATWIPEWEFVNTPPLQDSGLEVNIVTDS |
| | | | QYALGIIQAQPDSWTVNDIQKLVGKLNWASQIYPGIKNPDIVIYQYMDDLYVGSDLEIGQHRAADPLWKGPAKLLWKGEGAV |
| | | | VIQDNSDRQANFLGKIWPSHKGRAAPPFLWMGYELHPDKWTVQPIVLPEKFRKQNPDIVIYQYMDDLYVGSDLEINLLTQIG |
| | | | CTLNFPISPIETVPVKLKGPKEPFRDYVDRFYKTLRAEQASQEVYLKDQQLLGIWGCSGKLICTTAVPWAAAKKHQKEPPFL |
| | | | WMGYELHPDKWTVQPGTGPCTNVSTVQCTHGIRPVVSTQLKQNPDIVIYQYMDDLYVGSDLEIGQLGPAATLEEMMTACQGV |
| | | | GGPGHKARDQSLKPCVKLTPLCVTLNCTDLRNTGPGIRYPLLTFGWCFKLPVEPEKVEAKIIRDYGKQMAGDDCVASRQDED |
| | | | ILKALGPAATLEEMMTACQGVGGPGYKAAVDLSHFLREKGGLEGAAYAAKAYDTEVHNVWATHACVPTDPNPQEAAALSEGA |
| | | | TPQDLNTMLNTVGGHQAAMQQVDCSPGIWQLDCTHLEGKIILVAV |
| 375 | AA, | Env, | MDAMKRGLCCVLLLCGAVFVSARASRELERFAVNPGLLQLKGEAMHGQVDCSPGIWQLDCTHLCSATEKLWVTVYYGVPVWK |
| | AAA/ | Gag, | EATTTLPVGEIYKRWIILGLNKIVRMYSPTSINNETPGIRYQYNVLPQGWKGSPAIFAAPLWKGPAKLLWKGEGAVVIQDNS |
| | t-PA | Nef, | DIAAIVLPEKDSWTVNDIQKLVGKLNWASVYYRDSRDPLWKGPAKLLWKGEGAVSNFTSTTVKAACWWAGIKQEFGIPYVLP |
| | | Pol | EKDSWTVNDIQKLVGKLNWASQMVHQAISPRTLNAWVKVVEEKAFSPAAWETWWTEYWQATWIPEWEFVNTPPLQDSGLEVN |
| | | | IVTDSQYALGIIQAQPDSWTVNDIQKLVGKLNWASQTYPGIKNPDIVIYQYMDDLYVGSDLEIGQHRAADPLWKGPAKLLWK |
| | | | GEGAVVIQDNSDRQANFLGKIWPSHKGRAAPPFLWMGYELHPDKWTVQPIVLPEKFRKQNPDIVIYQYMDDLYVGSDLEINL |
| | | | LTQIGCTLNFPISPIETVPVKLKGPKEPFRDYVDRFYKTLRAEQASQEVYLKDQQLLGIWGCSGKLICTTAVPWAAAKKHQK |
| | | | EPPFLWMGYELHPDKWTVQPGTGPCTNVSTVQCTHGIRPVVSTQLKQNPDIVIYQYMDDLYVGSDLEIGQLGPAATLEEMMT |
| | | | ACQGVGGPGHKARDQSLKPCVKLTPLCVTLNCTDLRNTGPGIRYPLLTFGWCFKLPVEPEKVEAKIIRDYGKQMAGDDCVAS |
| | | | RQDEDILKALGPAATLEEMMTACQGVGGPGYKAAVDLSHFLREKGGLEGAAYAAKAYDTEVHNVWATHACVPTDPNPQEAAA |
| | | | LSEGATPQDLNTMLNTVGGHQAAMQQVDCSPGIWQLDCTHLEGKIILVAV |
| 376 | AA, | Env, | MNPSAAVIFCLILLGLSGTQGILDMAQPVGINTSTTCCYRFINKKIPKQRLESYRRTTSSHCPREAVIFKTKLDKEICADPT |
| | AAA/ | Gag, | QKWVQDFMKHLDKKTQTPKLASAGAASRELERFAVNPGLLQLKGEAMHGQVDCSPGIWQLDCTHLCSATEKLWVTVYYGVPV |
| | MCP-3 | Nef, | WKEATTTLPVGEIYKRWIILGLNKIVRMYSPTSINNETPGIRYQYNVLPQGWKGSPAIFAAPLWKGPAKLLWKGEGAVVIQD |
| | | Pol | NSDIAAIVLPEKDSWTVNDIQKLVGKLNWASVYYRDSRDPLWKGPAKLLWKGEGAVSNFTSTTVKAACWWAGIKQEFGIPYV |
| | | | LPEKDSWTVNDIQKLVGKLNWASQMVHQAISPRTLNAWVKVVEEKAFSPAAWETWWTEYWQATWIPEWEFVNTPPLQDSGLE |
| | | | VNIVTDSQYALGIIQAQPDSWTVNDIQKLVGKLNWASQIYPGIKNPDIVIYQYMDDLYVGSDLEIGQHRAADPLWKGPAKLL |
| | | | WKGEGAVVIQDNSDRQANFLGKIWPSHKGRAAPPFLWMGYELHPDKWTVQPIVLPEKFRKQNPDIVIYQYMDDLYVGSDLEI |
| | | | NLLTQIGCTLNFPISPIETVPVKLKGPKEPFRDYVDRFYKTLRAEQASQEVYLKDQQLLGIWGCSGKLICTTAVPWAAAKKH |
| | | | QKEPPFLWMGYELHPDKWTVQPGTGPCTNVSTVQCTHGIRPVVSTQLKQNPDIVIYQYMDDLYVGSDLEIGQLGPAATLEEM |
| | | | MTACQGVGGPGHKARDQSLKPCVKLTPLCVTLNCTDLRNTGPGIRYPLLTFGWCFKLPVEPEKVEAKIIRDYGKQMAGDDCV |
| | | | ASRQDEDILKALGPAATLEEMMTACQGVGGPGYKAAVDLSHFLREKGGLEGAAYAAKAYDTEVHNVWATHACVPTDPNPQEA |
| | | | AALSEGATPQDLNTMLNTVGGHQAAMQQVDCSPGIWQLDCTHLEGKIILVAV |
| 377 | AA, | Env, | MRKAAVSHWQQQSYLDSGIHSGATTTAPSLSASRELERFAVNPGLLQLKGEAMHGQVDCSPGIWQLDCTHLCSATEKLWVTV |
| | AAA/β- | Gag, | YYGVPVWKEATTTLPVGEIYKRWIILGLNKIVRMYSPTSINNETPGIRYQYNVLPQGWKGSPAIFAAPLWKGPAKLLWKGEG |
| | catenin | Nef, | AVVIQDNSDIAAIVLPEKDSWTVNDIQKLVGKLNWASVYYRDSRDPLWKGPAKLLWKGEGAVSNFTSTTVKAACWWAGIKQE |
| | | Pol | FGIPYVLPEKDSWTVNDIQKLVGKLNWASQMVHQAISPRTLNAWVKVVEEKAFSPAAWETWWTEYWQATWIPEWEFVNTPPL |
| | | | QDSGLEVNIVTDSQYALGIIQAQPDSWTVNDIQKLVGKLNWASQIYPGIKNPDIVIYQYMDDLYVGSDLEIGQHRAADPLWK |
| | | | GPAKLLWKGEGAVVIQDNSDRQANFLGKIWPSHKGRAAPPFLWMGYELHPDKWTVQPIVLPEKFRKQNPDIVIYQYMDDLYV |
| | | | GSDLEINLLTQIGCTLNFPISPIETVPVKLKGPKEPFRDYVDRFYKTLRAEQASQEVYLKDQQLLGIWGCSGKLICTTAVPW |
| | | | AAAKKHQKEPPFLWMGYELHPDKWTVQPGTGPCTNVSTVQCTHGIRPVVSTQLKQNPDIVIYQYMDDLYVGSDLEIGQLGPA |
| | | | ATLEEMMTACQGVGGPGHKARDQSLKPCVKLTPLCVTLNCTDLRNTGPGIRYPLLTFGWCFKLPVEPEKVEAKIIRDYGKQM |
| | | | AGDDCVASRQDEDILKALGPAATLEEMMTACQGVGGPGYKAAVDLSHFLREKGGLEGAAYAAKAYDTEVHNVWATHACVPTD |
| | | | PNPQEAAALSEGATPQDLNTMLNTVGGHQAAMQQVDCSPGIWQLDCTHLEGKIILVAV |
| 422 | AA, | Gag, | ICGHKAIGTVLVGPTPVNIIGRNLLTQLGCTLNFPISPIETVPVKLKPGMDGPKVKQWPLTEEKIKALDPLWKGPAKLLWKG |
| | AAY/ | Nef, | EGAVVIQDNSDIKVVPRRKAKIIRDYGKQMAAAYSDIAGTTSTLQEQITWMTNNPPIPVGEIYKRWIILGLNKIVRMYSPVS |
| | none | Pol | ILDIRQGPKEPFRDYVDRFYKTLRAEQASQEVKNWMTETLLVQNANPDCKTILKALGPAATLEEMMTACQGVGGPGHKARVL |
| | | | AEAMSQVTNSATLNKRTQDFWEVQLGIPHPAGLKKKKSNFTSTTVKAACWWAGIKQEFGIPYNPQSAYFSVPLDKEFRKYTA |
| | | | FTIPSINNEDTVLEEMNLPGKWKPKMIGGIGGFIKVRQYDQISKIGPENPYNTPIFAIKKKDSTKWAAGKKKYRLKHLVWVS |
| | | | RELERFAVNPGGKKKYRLKHLVWASRELERFAVNPGAEHLKTAVQMAVFIHNFKRKGGIGGAAGQMVHQAISPRTLNAWVKV |
| | | | VEEKAFSPEVIPMFSALAEGATPQDLNTMLNTVGGHQARWIILGLNKTVRMYSPVSILDIRQGPKEPFRDYVDRFYKTLRAE |
| | | | QASQEVKNWMTETLLVQNANPDCKTILKALGPAATLEEMMTACQGVGGPGHKARVLAEAMSQVTNSATQLKGEAMHGQVDCS |
| | | | PGIWQLDCTHLEGKVILVAVHVASGYIEAEVIPAETGQETAYFLLKLAGR |
| 423 | AA/ | Gag, | SNFTSTTVKVACWWAGIKQEFGIPYAASNFTSTTVKAACWWAGVKQEFGIPYAASNFTSTTVKAACWWAGIKQEFGIPYPLR |
| | none | Nef, | PMTYKAAVDLSHFLKEKGGLEGPLRPMTYKAAVDLSFFLKEKGGLEGPLRPMTYKGAFDLSFFLKEKGGLEGPLRPMTYKAA |
| | | Pol | FDLSFFLKEKGGLEGPLRPMTYKAAFDLSHFLKEKGGLEGPLRPMTYKGALDLSHFLKEKGGLEGQLKGEAMHGQVDCSPGI |
| | | | WQLDCTHLEEKIILVAVHVASGYIEAEVIPAETGQETAYMVHQAISPRTLNAWVKVVEEKAFSPLDCTHLEGKVILVAVHVA |
| | | | SGYIEAEICGHKAIGTVLVGPTPVNIIGRNLLTQIGCTLNFPISPIETVPVKLKDPLWKGPAKLLWKGEGAVVIQDNSDIDP |
| | | | LWKGPAKLLWKGEGVVVIQDNSDIMVHQAISPRTLNALVKVVEEKAFSPICGHKAIGTVLVGSTPVNIIGRNLL |

TABLE K
HIV-1 sequence segments that may be excluded from the present fusion proteins

| SEQ | | | | |
|---|---|---|---|---|
| ID | HIV-1 | | | |
| NO: | Gene | start | end | SEQUENCE |
| 437 | Env | 1 | 27 | MRVKEKYQHLWRWGWRWGTMLLGMLMI |
| 438 | Env | 53 | 58 | FCASDA |
| 439 | Env | 84 | 112 | VVLVNVTENFNMWKNDMVEQMHEDIISLW |
| 440 | Env | 138 | 234 | NTNSSSGRMIMEKGEIKNCSFNISTSIRGKVQKEYAFFYKLDIIPIDNDTTSYKLTSCNTSVITQACPKVSFEPIPIHYC |
| | | | | APAGFAILKCNNKTFN |
| 441 | Env | 269 | 474 | EVVIRSVNFTDNAKTIIVQLNTSVEINCTRPNNNTRKRIRIQRGPGRAFVTIGKIGNMRQAHCNISRAKWNNTLKQIASK |
| | | | | LREQFGNNKTIIFKQSSGGDPEIVTHSFNCGGEFFYCNSTQLFNSTWFNSTWSTEGSNNTEGSDTITLPCRIKQIINMWQ |
| | | | | KVGKAMYAPPISGQIRCSSNITGLLLTRDGGNSNNESEIFRPGGGD |
| 442 | Env | 490 | 501 | KIEPLGVAPTKA |
| 443 | Env | 611 | 856 | NASWSNKSLEQIWNHTTWMEWDREINNYTSLIHSLIEESQNQQEKNEQELLELDKWASLWNWFNITNWLWYIKLFIMIVG |
| | | | | GLVGLRIVFAVLSIVNRVRQGYSPLSFQTHLPTPRGPDRPEGIEEEGGERDRDRSIRLVNGSLALIWDDLRSLCLFSYHR |
| | | | | LRDLLLIVTRIVELLGRRGWEALKYWWNLLQYWSQELKNSAVSLLNATAIAVAEGTDRVIEVVQGACRAIRHIPRRIRQG |
| | | | | LERILL |
| 444 | Gag | 1 | 30 | MGARASVLSGGELDRWEKIRLRPGGKKKYK |
| 445 | Gag | 54 | 127 | SEGCRQILGQLQPSLQTGSEELRSLYNTVATLYCVHQRIEIKDTKEALDKIEEEQNKSKKKAQQAAADTGHSNQ |
| 446 | Gag | 138 | 146 | IQGQMVHQA |
| 447 | Gag | 370 | 428 | VTNSATIMMQRGNFRNQRKIVKCFNCGKEGHTARNCRAPRKKGCWKCGKEGHQMKDCTE |
| 448 | Gag | 445 | 500 | PGNFLQSRPEPTAPPEESFRSGVETTTPPQKQEPIDKELYPLTSLRSLFGNDPSSQ |
| 449 | Nef | 1 | 63 | MGGKWSKSSVIGWPTVRERMRRAEPAADRVGAASRDLEKHGAITSSNTAATNAACAWLEAQEE |
| 450 | Nef | 103 | 116 | LIHSQRRQDILDLWIYH |
| 451 | Nef | 155 | 206 | PGVRYPLTFGWCYKLVPVEPDKIEEANKGENTSLLHPVSLHGMDDPEREVLEWRFDSRLAFHHVARELHPEYFKNC |
| 452 | Pol | 1 | 55 | FFREDLAFLQGKAREFSSEQTRANSPTRRELQVWGRDNNSPSEAGADRQGTVSFN |
| 453 | Pol | 118 | 128 | ILIEICGHKAI |
| 454 | Pol | 321 | 325 | KILEP |
| 455 | Pol | 355 | 366 | TKIEELRQHLLR |
| 456 | Pol | 432 | 541 | QLCKLLRGTKALTEVIPLTEEAELELAENREILKEPVHGVYYDPSKDLIAEIQKQGQGQWTYQIYQEPFKNLKIGKYARM |
| | | | | RGAHTNDVKQLTEAVQKITTESIVIWGKT |
| 457 | Pol | 607 | 641 | LGKAGYVTNRGRQKVVTLTDTTNQKTELQAIYLAL |
| 458 | Pol | 667 | 682 | QSESELVNQIIEQLIK |
| 459 | Pol | 709 | 746 | GIRKVLFLDGIDKAQDEHEKYHSNWRAMASDFNLPPV |
| 460 | Pol | 828 | 833 | IHTDNG |
| 461 | Pol | 921 | 930 | TDIQTKELQK |

### 3. Polynucleotides Encoding the Fusion Polypeptides

Provided are polynucleotides encoding the fusion polypeptides, described herein, vectors comprising such polynucleotides, and host cells (e.g., human cells, mammalian cells, yeast cells, plant cells, insect cells, bacterial cells, e.g., E. coli) comprising such polynucleotides or expression vectors. Provided herein are polynucleotides comprising nucleotide sequence(s) encoding any of the fusion polypeptides provided herein, as well as expression cassettes and vector(s) comprising such polynucleotide sequences, e.g., expression vectors for their efficient expression in host cells, e.g., mammalian cells. In various embodiments, the polynucleotide is a DNA, a cDNA, an mRNA, a self-amplifying RNA (SAM), a self-replicating RNA, or a self-amplifying replicon RNA (RepRNA). In some embodiments, the polynucleotide comprises an alphavirus self-replicating or self-amplifying replicon RNA (RepRNA). Self-replicating RNA and self-amplifying replicon RNA as modes of vaccine delivery are described, e.g., by Tews, et al., Methods Mol Biol. (2017) 1499:15-35; Demoulins, et al., Methods Mol Biol. (2017) 1499:37-75; Englezou, et al., Mol Ther Nucleic Acids. (2018) 12:118-134; McCollough, et al., Vaccines (Basel). (2014) 2(4):735-54; and McCollough, et al., Mol Ther Nucleic Acids. (2014) 3:e173.

The terms “polynucleotide” and “nucleic acid molecule” interchangeably refer to a polymeric form of nucleotides and includes both sense and anti-sense strands of RNA, cDNA, genomic DNA, and synthetic forms and mixed polymers of the above. As used herein, the term nucleic acid molecule may be interchangeable with the term polynucleotide. In some embodiments, a nucleotide refers to a ribonucleotide, deoxynucleotide or a modified form of either type of nucleotide, and combinations thereof. The terms also include without limitation, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-biased polynucleotides for improved expression in a desired viral expression vector or host cell.

A “substitution,” as used herein, denotes the replacement of one or more amino acids or nucleotides by different amino acids or nucleotides, respectively.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location. “Isolated nucleic acid encoding an polypeptide segment or encoding a fusion polypeptide” refers to one or more nucleic acid molecules encoding such polypeptide segments or fusion polypeptides, including such nucleic acid molecule(s) in a single vector or separate vectors, and such nucleic acid molecule(s) present at one or more locations in a host cell.

A “polynucleotide variant,” as the term is used herein, is a polynucleotide that typically differs from a polynucleotide specifically disclosed herein in one or more substitutions, deletions, additions and/or insertions. Such variants may be naturally occurring or may be synthetically generated, for example, by modifying one or more of the polynucleotide sequences described herein and evaluating one or more biological activities of the encoded polypeptide as described herein and/or using any of a number of techniques well known in the art.

In some embodiments, the nucleic acid molecule is codon-biased to enhance expression in a desired host cell, e.g., in human cells, mammalian cells, yeast cells, plant cells, insect cells, or bacterial cells, e.g., E. coli cells. Accordingly, provided are polynucleotides encoding a fusion polypeptide, described herein, wherein the polynucleotides are codon-biased, comprise replacement heterologous signal sequences, and/or have mRNA instability elements eliminated. Methods to generate codon-biased nucleic acids can be carried out by adapting the methods described in, e.g., U.S. Pat. Nos. 5,965,726; 6,174,666; 6,291,664; 6,414,132; and 6,794,498. Preferred codon usage for expression of the fusion polypeptides comprising HIV-1 polypeptide segments from desired viral expression vectors and/or in desired host cells is provided, e.g., at kazusa.or.jp/codon/; and genscript.com/tools/codon-frequency-table.

In some embodiments, the polynucleotide encoding a fusion polypeptide, as described herein, has at least 80%, at least 85%, at least 90%, at least 91%, at least 92%, at least 93%, at least 94%, at least 95%, at least 96%, at least 97%, at least 98%, at least 99% identical, or 100% identical to a nucleic acid sequence selected from the group consisting of SEQ ID NOs: 414-418, as provided in Table L.

As appropriate, in certain embodiments, the 3′-end of the polynucleotide encoding the fusion polypeptides described herein comprises one or multiple tandem stop codons, e.g., two or more tandem TAG (“amber”), TAA (“ochre”) or TGA (“opal” or “umber”) stop codons. The multiple tandem stop codons can be the same or different.

Further provided are expression cassettes, comprising a polynucleotide encoding a fusion polypeptide, as described herein, operably linked to one or more regulatory sequences. In some embodiments, the polynucleotide is operably linked to and under the control of a constitutive promoter. In some embodiments, the promoter is selected from cytomegalovirus major immediate-early (CMV), the CMV enhancer fused to the chicken beta-actin promoter (CAG), human elongation factor-1α (HEF-1α), mouse cytomegalovirus (mouse CMV), Chinese hamster elongation factor-1α (CHEF-1α), and phosphoglycerate kinase (PGK).

TABLE L
Polynucleotides encoding fusion polypeptides (“REKR” is SEQ ID NO: 382)

| | HIV-1 | |
|---|---|---|
| SEQ ID | GENES/ | |
| NO: | FEATURES | SEQUENCE |
| 414 | Gag, Nef | ATGGGAGCTAGAGCTAGCGTGCTGAGCGGAGGAGAACTCGATCGCTGGGAAAAGATCAGACTGAGACCAGGAGGCAAGA |
| | | AGAAGTACAGACTGAAGCACATCGTCTGGGCTTCTAGAGAACTGGAAAGATTCGCCGTGAATCCAGGACTGCTGGAAAC |
| | | ACTGAAGCACATTGTCTGGGCTAGCAGAGAACTGGAGAGATTTGCCGTGAATCCAGGACTGCTGGAAACAGCAGCTATC |
| | | TCTCCTAGAACACTGAACGCTTGGGTGAAAGTGGTGGAGGAAAAGGCCTTTAGCCCAGAAGTGATCCCTATGTTTAGCG |
| | | CCCTGTCAGAAGGAGCTACACCTCAGGATCTGAACACCATGCTGAACACAGTGGGAGGACATCAGGCAGCTATGCAGAT |
| | | GCTGAAGGAGACAATTAACGAAGAAGCCGCCGAGTGGGATAGACTGCATCCAGTGCACGCAGGACCTATTGCTCCAGGA |
| | | CAGATGAGAGAGCCTAGAGGAAGCGATATCGCAGGAACAACATCTACACTGCAGGAGCAGATCGGTTGGATGACCAATA |
| | | ATCCTCCTATCCCAGTGGGCGAAATCTATAAGCGCTGGATCATCCTGGGACTGAACAAGATCGTGAGGATGTACAGCCC |
| | | TACCAGCATCCTGGATATCAGACAGGGACCTAAGGAGCCTTTCAGAGATTACGTGGACAGGTTCTACAAGACACTGAGA |
| | | GCCGAACAGGCTTCTCAGGAGGTGAAGAATTGGATGACCGAGACACTGCTGGTGCAGAACGCTAATCCAGATTGCAAGA |
| | | CAATTCTGAAAGCTCTGGGACCAGCCGCTACACTGGAAGAGATGATGACCGCTTGTCAGGGAGTGGGAGGACCAGGACA |
| | | TAAAGCTAGAGTGCTGGCAGAAGCCATGTCTCAGGAAGAAGTGGGATTCCCAGTGAAACCTCAGGTGCCTCTGAGACCT |
| | | ATGACCTTTAAGGGAGCTCTGGACCTGTCTCACTTCCTGAGAGAAAAGGGAGGACTGGAAGGAACACAGGGATTTTTCC |
| | | CAGATCAGAATTACACACCAGAGCCAGGAATCAGATTCCCTCTGACATTCGGTTGGTGCTTCAAACTGGTGCCTCTG |
| 415 | PolEnv | GGAACAGTGCTGGTGGGACCTACTCCAGTGAATATCATCGGAAGGAACCTGCTGACACAGATTGGTTGTACCCTGAACT |
| | | TCCCTATCTCTCCTATCGAGACAGTGCCAGTGAAACTGAAGCCAGGAATGGATGGACCTAAAGTCAAGCAGTGGCCTCT |
| | | GACAGAAGAGAAGATCAAAGCCCTGGTGGAGATTTGCACCGAGATGGAGAAGGAGGGAAAGATCAGCAAGATCGGCCCA |
| | | GAGAATCCTTACAACACCCCAGTGTTCGCCATCAAGAAGAAGGATAGCACCAAGTGGAGAAAGCTGGTGGATTTCAGGG |
| | | AGCTGAACAAGAGAACCCAGGATTTTTGGGAGGTGCAGCTGGGTATTCCACATCCTGCCGGACTGAAAAAGAAGAAAAG |
| | | CGTGACAGTGCTGGACGTGGGAGACGCTTATTTCAGCGTGCCTCTGGATAAGGACTTCAGAAAGTACACCGCCTTCACC |
| | | ATCCCTTCTATCAACAACGAGACCCCAGGAATCAGATACCAGTACAACGTGCTGCCTCAAGGTTGGAAAGGATCTCCAG |
| | | CCATCTTTCAGAGCAGCATGACAACAGTGAAGGCAGCTTGTTGGTGGGCAGGAATTAAGCAGGAGTTCGGCATCCCTTA |
| | | CAATCCTCAGTCTCAGGGAGTGGTGGAATCTATGAACAAGGAGCTGAAGAAGATCATCGGACAGGTGAGAGATCAGGCC |
| | | GAACATCTGAAGACAGCAGTGCAAATGGCCGTGTTCATCCACAACTTCAAGAGAAAGGGCGGCATTGGAGGCTATTCTG |
| | | CCGGAGAGAGAATTGTGGACATCATCAACGTGTCAACAGTCCAGTGTACACACGGAATCAGACCAGTCGTGTCTACACA |
| | | ACTGCTGCTGAACGGATCTCTGGCCGAAGAGAAGAGAAGAGTGGTGCAGAGAGAGAAAAGAGCAGTGGGAATCGGAGCT |
| | | ATGTTTCTGGGATTTCTGGGCGCAGCAGGATCTACAATGGGAGCAGCTTCTATCACACTGACAGTGCAGGCTAGACAAC |
| | | TGCTGAGCGGAATTGTGCAGCAGCAGAATAACCTGCTGAGAGCTATCGAAGCTCAGCAACATCTGCTGCAACTCACCGT |
| | | CTGGGGAATTAAGCAACTGCAAGCTAGAGTGCTGGCAGTGGAAAGATACCTGAAGGATCAGCAACTGCTGGGAATTTGG |
| | | GGTTGCTCAGGCAAGCTGATTTGCACAACCGTGGCCAAAGAGATTGTGGCTTCTTGCGACAAGTGTCAGCTGAAAGGAG |
| | | AAGCTATGCACGGACAAGTGGATTGTTCTCCAGGAATTTGGCAGCTGGATTGTACACACCTGGAGGGAAAGATTATTCT |
| | | GGTGGCAGTGCACGTGGCCAGCGGATATATTGAAGCCGAGGTGATTCCAGCAGAAACAGGACAGGAAACAGCCTATTTT |
| | | CTCCTGAAACTGGCAGGTAGGTGGCCAGTGAAAACCCTCTGGGTGACAGTGTACTACGGAGTCCCAGTCTGGAAAGAAG |
| | | CAGCTTTCCCTCAGATTACTCTCTGGCAGAGACCTCTGGTGACAATCAAGATCGGCGGACAGCTGAAAGAAGCTCTGCT |
| | | GGATACAGGAGCAGACGATACAGTGCTGGAAGAAATGAACCTGCCAGGTAGATGGAAGCCTAAGATGATCGGAGGCATC |
| | | GGAGGATTCATCAAGGTGAGACAGTACGACCAAGCAGCAGCAGCTCATAACGTCTGGGCTACACACGCTTGCGTGCCTA |
| | | CAGATCCTAATCCTCAGGAAGCCATCACCAAGATCCAGAATTTCAGGGTGTACTACAGGGACAGCAGAGATCCTCTCTG |
| | | GAAAGGACCAGCTAAACTGCTGTGGAAAGGAGAAGGAGCAGTGGTGATCCAGGATAACAGCGACATCAAGGTGGTGCCT |
| | | AGAAGAAAGGCCAAGATCATCAGGGACTACGGAAAGCAAATGGCAGGAGACGATTGCGTGGCTTCTAGACAGGACGAGG |
| | | ATCCCAAGTTCAAGCTGCCTATTCAGAAGGAGACTTGGGAGACTTGGTGGACAGAGTATTGGCAAGCAACTTGGATCCC |
| | | CGAGTGGGAATTTGTGAATACCCCTCCTCTGGTCAAGCTCTGGTATCAGCTGGAAAAGGAGCCTATCGTGGGAGCCGAA |
| | | ACATTTTACGTGGACGGAGCAGCTAATAGAGAGACAAAAGCCGCCAAGGAGAAAGTGTATCTGGCTTGGGTGCCAGCTC |
| | | ATAAAGGAATCGGAGGAAACGAGCAGGTGGATAAACTGGTGTCTTGGGGCTTTACCACACCAGATAAGAAGCACCAGAA |
| | | GGAGCCACCATTTCTCTGGATGGGATACGAACTGCACCCAGATAAGTGGACAGTCCAGCCTATTGTGCTGCCAGAAAAG |
| | | GACTCTTGGACAGTGAACGACATCCAGAAACTGGTGGGAAAGCTGAATTGGGCCTCTCAGATCTACCCAGGCATCAAGG |
| | | TGATCGTGATCTACCAGTACATGGACGATCTGTACGTGGGATCAGATCTGGAGATCGGACAGCACAGAATGAGGGACAA |
| | | TTGGAGAAGCGAGCTGTACAAGTACAAGGTGGTG |
| 416 | Env, | TACCAGTATAACGTGCTGCCTCAGGGAGCTTCTAGAGAACTGGAGAGATTCGCAGTGAACCCAGGACTCCTC |
| | Gag, | TGGATTATCCTGGGACTGAACAAGATCGTGAGGATGTACTCTCCTACCTCTATTGCCGCTAGAACACTGAAC |
| | Nef, | GCTTGGGTGAAGGTCTTCCTCTGGATGGGATACGAACTGCATCTGACCTTTGGTTGGTGCTTTAAGCTCCCT |
| | Pol | CTCTGGAAAGGACCAGCTAAGCTGGTGACAGTGTATTACGGAGTGCCAGTGGCAGCTCTCCTCTGGAAAGGA |
| | | GAAGGAGCAGTGGCAGCAGCTAAACTGGTGGGAAAGCTGAATTGGGCCAAACTCCTCTGGAAGGGAGAAGGA |
| | | GCCACCCTGAATTTTCCTATCAGCCCTATTTGGCAGGCTACTTGGATTCCAGAGTGGAAAGCAGCTTGTTGG |
| | | TGGGCAGGAATCAGACAGGCCAACTTCCTGGGCAAGATTTGGCCTTCTCACAAAGGAAGAAACGTCTGGGCT |
| | | ACACACGCTTGCGTGGCAGCAGAAATGATGACAGCTTGTCAGGGAGTGTCTACAGTCCAGTGTACACACGGA |
| | | ATCGCAGCTAAACAGATGGCAGGAGACGATTGCGTGGCAGCTTGGCAGCTGGATTGTACACACCTGGAGTAC |
| | | AAGGCAGCAGTGGATCTGTCTCACTTTCTGAGAGAAAAAGGAGGACTGGAAGGAGCAGCTTACTACATGGAC |
| | | GATCTGTACGTGGGATCAGGACAGGTGGATTGTTCACCAGGAATCGCTACACTGGAGGAAATGATGACCGCA |
| | | GAACTGCATCCAGATAAGTGGACCGTCTGGACAGTGAACGATATCCAGAAGCTGGGCATTTGGGGTTGTAGC |
| | | GGAAAACTGACCGTGAACGATATCCAGAAGCTGGTGATCGTGACCGATTCTCAGTACGCTCTGTACGTGGAC |
| | | AGATTCTACAAGACCCTGTACGTGGACAGGTTCTACAAGACACTGAGAGCCGAACAGGCTTCTCAGGAAGTG |
| | | GATCTGAACACCATGCTGAACACCGTGAAACTGACACCTCTCTGCGTGACACTGTATCAGTACATGGACGAC |
| | | CTGTACGTGGTGATCTACCAGTACATGGACGATCTCTGGATCATCCTGGGACTGAACAAGATCG |
| 417 | Env, | AGAACACTGAACGCTTGGGTGAAGGTGAGAGAGAAGAGAGACCTGAACACCATGCTGAACACCGTGAGAGAA |
| | Gag, | AAGAGGTGGATCATCCTGGGACTGAACAAGATCAGGGAGAAGAGGTACGTGGACAGGTTCTACAAGACACTG |
| | Nef, | AGAGAGAAGAGAGCCACACTGGAAGAGATGATGACCGCTAGAGAGAAGAGAGAGATGATGACCGCTTGTCAG |
| | Pol/ | GGAGTGAGAGAGAAGAGAACCCTGAACTTCCCCATCTCTCCTATCAGGGAGAAGAGGTACCAGTACAACGTG |
| | REKR | CTGCCTCAGGGAAGAGAAAAGAGAGTGATCTACCAGTACATGGACGACCTGAGAGAGAAGAGGTACCAGTAC |
| | linkers | ATGGACGATCTGTACGTGAGGGAGAAGAGATACATGGACGACCTGTACGTGGGATCAAGAGAGAAGAGATTC |
| | | CTCTGGATGGGCTACGAGCTGCATAGAGAGAAGAGAGAGCTGCACCCAGATAAGTGGACAGTGAGAGAAAAG |
| | | CGCTGGACAGTGAACGACATCCAGAAGCTGAGAGAGAAGAGGACAGTGAACGACATCCAGAAGCTGGTGAGA |
| | | GAGAAGAGGAAGCTGGTGGGAAAACTGAATTGGGCTAGGGAAAAAAGGTGGCAGGCTACTTGGATTCCAGAG |
| | | TGGAGAGAGAAGAGGATCGTGACAGATAGCCAGTACGCTCTGAGAGAGAAAAGAGGACAGGTGGATTGCTCT |
| | | CCAGGAATCAGAGAGAAGAGATGGCAGCTGGATTGTACACACCTGGAGAGAGAGAAGAGGAAAGCAGCTTGT |
| | | TGGTGGGCAGGAATTCGGGAAAAAAGACCTCTCTGGAAAGGACCAGCCAAGCTGAGAGAGAAGAGAAAACTC |
| | | CTCTGGAAGGGCGAAGGAGCTAGAGAAAAGAGACTCCTCTGGAAAGGAGAAGGCGCAGTGAGAGAGAAGAGA |
| | | AAACAGATGGCCGGAGACGATTGCGTGAGAGAAAAGAGAGTGACCGTGTATTACGGAGTGCCAGTGAGAGAA |
| | | AAGAGAAACGTCTGGGCTACACACGCTTGCGTGAGAGAGAAGAGAAAGCTGACACCTCTGTGCGTGACACTG |
| | | AGAGAAAAGAGAAGCACCGTGCAGTGTACACACGGAATTAGGGAGAAGAGAGGCATTTGGGGTTGTTCAGGA |
| | | AAGCTGAGAGAGAAGAGGCTGACATTCGGTTGGTGTTTCAAGCTGAGGGAGAAGAGAGCCTCTAGAGAACTG |
| | | GAGAGATTCGCAGTGAATCCAGGACTGCTGAGAGAAAAGCGCTGGATTATCCTGGGACTGAACAAGATCGTG |
| | | AGGATGTACAGCCCTACAAGCATCAGAGAGAAGAGGTACGTGGACAGATTCTACAAGACCCTGAGAGCCGAA |
| | | CAGGCATCTCAGGAAGTGAGAGAGAAGAGAAGGCAGGCTAACTTCCTGGGAAAGATTTGGCCTAGCCACAAG |
| | | GGAAGAAGAGAGAAGAGATACAAGGCCGCAGTGGATCTGTCTCACTTTCTGAGAGAGAAAGGAGGACTGGAA |
| | | GGAGGA |
| 418 | Env, | ATGGCTCCTAGAAGCGCTAGAAGACCTCTGCTGCTGCTGCTGCTGCTGCTGCTGCTGGGACTGATGCATTGC |
| | Gag, | GCTTCAGCAGCTATGTTCATGGTGAAGAACGGCAACGGAACAGCTTGTATCATGGCCAATTTCAGCGCCGCT |
| | Nef, | TTTAGCGTGAATTACGACACCAAGAGCGGACCTAAGAACATGACACTGGATCTGCCTTCAGACGCTACAGTG |
| | Pol/ | GTGCTGAATAGAAGCTCTTGCGGAAAGGAGAATACCTCCGATCCTTCTCTGGTGATCGCTTTTGGCAGAGGA |
| | REKR | CACACACTGACACTGAACTTCACCAGAAACGCCACCAGATACTCAGTGCAGCTGATGAGCTTCGTGTACAAC |
| | linkers; | CTGAGCGATACCCATCTGTTTCCTAACGCTAGCAGCAAGGAGATCAAGACAGTGGAGTCTATCACCGACATC |
| | LAMP-1 | AGAGCCGATATCGACAAGAAATACCGCTGCGTGTCAGGAACACAGGTGCACATGAACAACGTGACAGTGACA |
| | N-term & | CTGCACGACGCCACAATTCAGGCCTATCTGAGCAATAGCAGCTTTAGCAGAGGCGAAACTAGGTGTGAGCAG |
| | C-term | GATAGACCTTCTCCTACAACAGCTCCTCCAGCTCCTCCTTCTCCTTCTCCTTCTCCAGTGCCTAAATCTCCT |
| | signal | AGCGTGGATAAGTACAACGTGAGCGGAACAAACGGCACTTGTCTGCTGGCTTCTATGGGACTGCAGCTGAAT |
| | sequences | CTGACATACGAGAGGAAGGACAACACCACAGTGACAAGACTGCTGAACATCAACCCCAACAAAACAAGCGCT |
| | | AGCGGATCTTGCGGAGCTCATCTGGTGACACTGGAACTGCATTCAGAGGGAACAACAGTGCTGCTGTTTCAG |
| | | TTCGGAATGAACGCCTCTAGCAGCAGATTCTTCCTGCAGGGTATTCAGCTGAATACACTGCTGCCAGATGCT |
| | | AGAGATCCAGCCTTTAAAGCCGCTAATGGATCTCTGAGAGCTCTGCAGGCTACAGTGGGAAATAGCTACAAG |
| | | TGCAACGCCGAAGAACACGTGAGAGTGACAAAAGCCTTCAGCGTGAACATCTTTAAGGTCTGGGTGCAGGCA |
| | | TTTAAAGTGGAGGGAGGCCAGTTTGGAAGCGTCGAAGAGTGTCTGCTGGACGAAAATAGCCTGGAAGACATC |
| | | AGAACACTGAACGCTTGGGTGAAGGTGAGAGAGAAGAGAGACCTGAACACCATGCTGAACACCGTGAGAGAA |
| | | AAGAGGTGGATCATCCTGGGACTGAACAAGATCAGGGAGAAGAGGTACGTGGACAGGTTCTACAAGACACTG |
| | | AGAGAGAAGAGAGCCACACTGGAAGAGATGATGACCGCTAGAGAGAAGAGAGAGATGATGACCGCTTGTCAG |
| | | GGAGTGAGAGAGAAGAGAACCCTGAACTTCCCCATCTCTCCTATCAGGGAGAAGAGGTACCAGTACAACGTG |
| | | CTGCCTCAGGGAAGAGAAAAGAGAGTGATCTACCAGTACATGGACGACCTGAGAGAGAAGAGGTACCAGTAC |
| | | ATGGACGATCTGTACGTGAGGGAGAAGAGATACATGGACGACCTGTACGTGGGATCAAGAGAGAAGAGATTC |
| | | CTCTGGATGGGCTACGAGCTGCATAGAGAGAAGAGAGAGCTGCACCCAGATAAGTGGACAGTGAGAGAAAAG |
| | | CGCTGGACAGTGAACGACATCCAGAAGCTGAGAGAGAAGAGGACAGTGAACGACATCCAGAAGCTGGTGAGA |
| | | GAGAAGAGGAAGCTGGTGGGAAAACTGAATTGGGCTAGGGAAAAAAGGTGGCAGGCTACTTGGATTCCAGAG |
| | | TGGAGAGAGAAGAGGATCGTGACAGATAGCCAGTACGCTCTGAGAGAGAAAAGAGGACAGGTGGATTGCTCT |
| | | CCAGGAATCAGAGAGAAGAGATGGCAGCTGGATTGTACACACCTGGAGAGAGAGAAGAGGAAAGCAGCTTGT |
| | | TGGTGGGCAGGAATTCGGGAAAAAAGACCTCTCTGGAAAGGACCAGCCAAGCTGAGAGAGAAGAGAAAACTC |
| | | CTCTGGAAGGGCGAAGGAGCTAGAGAAAAGAGACTCCTCTGGAAAGGAGAAGGCGCAGTGAGAGAGAAGAGA |
| | | AAACAGATGGCCGGAGACGATTGCGTGAGAGAAAAGAGAGTGACCGTGTATTACGGAGTGCCAGTGAGAGAA |
| | | AAGAGAAACGTCTGGGCTACACACGCTTGCGTGAGAGAGAAGAGAAAGCTGACACCTCTGTGCGTGACACTG |
| | | AGAGAAAAGAGAAGCACCGTGCAGTGTACACACGGAATTAGGGAGAAGAGAGGCATTTGGGGTTGTTCAGGA |
| | | AAGCTGAGAGAGAAGAGGCTGACATTCGGTTGGTGTTTCAAGCTGAGGGAGAAGAGAGCCTCTAGAGAACTG |
| | | GAGAGATTCGCAGTGAATCCAGGACTGCTGAGAGAAAAGCGCTGGATTATCCTGGGACTGAACAAGATCGTG |
| | | AGGATGTACAGCCCTACAAGCATCAGAGAGAAGAGGTACGTGGACAGATTCTACAAGACCCTGAGAGCCGAA |
| | | CAGGCATCTCAGGAAGTGAGAGAGAAGAGAAGGCAGGCTAACTTCCTGGGAAAGATTTGGCCTAGCCACAAG |
| | | GGAAGAAGAGAGAAGAGATACAAGGCCGCAGTGGATCTGTCTCACTTTCTGAGAGAGAAAGGAGGACTGGAA |
| | | GGAGGAAGCGAGTTTACCCTGATTCCAATTGCCGTGGGAGGAGCTCTGGCAGGACTGGTGATTGTGCTGATC |
| | | GCATACCTGGTGGGAAGAAAGAGATCTCACGCCGGATATCAGACCATC |

### 4. Vectors and Host Cells

Further provided are vectors comprising one or more polynucleotides encoding one or more of the fusion polypeptides, described herein, or an expression cassette comprising such polynucleotides. A vector can be of any type, for example, a recombinant vector such as an expression vector. Vectors include without limitation, plasmids, cosmids, bacterial artificial chromosomes (BAC) and yeast artificial chromosomes (YAC) and vectors derived from bacteriophages or plant or animal (including human) viruses. Vectors can comprise an origin of replication recognized by the proposed host cell and in the case of expression vectors, promoter and other regulatory regions recognized by the host cell. In additional embodiments, a vector comprises one or more polynucleotides encoding one or more fusion polypeptides of the disclosure operably linked to a promoter and optionally additional regulatory elements. Certain vectors are capable of autonomous replication in a host into which they are introduced (e.g., vectors having a bacterial origin of replication can replicate in bacteria). Other vectors can be integrated into the genome of a host upon introduction into the host, and thereby are replicated along with the host genome. Vectors include without limitation, those suitable for recombinant production of the fusion polypeptides disclosed herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Some vectors are suitable for delivering the nucleic acid molecule or polynucleotide of the present application. Certain vectors are capable of directing the expression of nucleic acids to which they are operatively linked. Such vectors are referred to herein as expression vectors.

The term “operably linked” refers to two or more nucleic acid sequence elements that are usually physically linked and are in a functional relationship with each other. For instance, a promoter is operably linked to a coding sequence if the promoter is able to initiate or regulate the transcription or expression of a coding sequence, in which case, the coding sequence should be understood as being “under the control of” the promoter.

The choice of the vector is dependent on the recombinant procedures followed and the host used. Introduction of vectors into host cells can be effected by inter alia calcium phosphate transfection, DEAE-dextran-mediated transfection, lipofectamine transfection, electroporation, virus infection, or via administration to a subject, as described herein. Vectors may be autonomously replicating or may replicate together with the chromosome into which they have been integrated. In certain embodiments, the vectors contain one or more selection markers. The choice of the markers may depend on the host cells of choice. These include without limitation, kanamycin, neomycin, puromycin, hygromycin, zeocin, thymidine kinase gene from Herpes simplex virus (HSV-TK), and dihydrofolate reductase gene from mouse (dhfr). Vectors comprising one or more nucleic acid molecules encoding the fusion polypeptides described herein, operably linked to one or more nucleic acid molecules encoding proteins or peptides that can be used to isolate the fusion polypeptides (“purification tags”), are also covered by the disclosure. These proteins or peptides include without limitation, FLAG-tag (DYKDDDDKL; SEQ ID NO: 436), glutathione-S-transferase, maltose binding protein, metal-binding polyhistidine, green fluorescent protein, luciferase and beta-galactosidase.

In other embodiments, the vector that is used is pcDNA™ 3.1+(ThermoFisher, MA).

In some embodiments, the vector is viral vector. As appropriate, the viral vector can be a DNA virus or a RNA virus, including a self-replicating RNA virus. Self-replicating RNA viruses include Alphaviruses, and are described, e.g., in Lundstrom, Molecules. (2018) 23(12). pii: E3310 (PMID: 30551668); and Ljungberg, et al., Expert Rev Vaccines. (2015) 14(2):177-94). In various embodiments, the viral vector is from a virus selected from the group consisting of adenovirus, adeno-associated virus, arenavirus, alphavirus, self-replicating alphavirus, poxvirus, cytomegalovirus, rhabdovirus, vesicular stomatitis virus, flavivirus, maraba virus and vaccinia virus. In some embodiments, the viral vector is from a viral family selected from the group consisting of: Adenoviridae (e.g., Adenovirus, adeno-associated virus), Arenaviridae (e.g., lymphocytic choriomeningitis mammarenavirus, Cali mammarenavirus (a.k.a., Pichinde mammarenavirus), Herpesviridae (e.g., Cytomegalovirus, Herpesvirus, e.g., HSV-1), Parvoviridae (e.g., Parvovirus H1), Poxviridae (e.g. Vaccinia virus, e.g. modified vaccinia Ankara (MVA)), Paramyxoviridae (e.g. measles virus), Flaviviridae (e.g. Yellow fever virus), Reoviridae (e.g., Reovirus), Picornaviridae (e.g., Coxsackievirus, Seneca Valley Virus, Poliovirus), Paramyxoviridae (e.g., Measles virus, Newcastle disease virus (NDV)), Rhabdoviridae (e.g., Vesiculovirus, including Maraba vesiculovirus and Vesicular stomatitis virus (VSV)), Togaviridae (e.g., Alphavirus, e.g., self-replicating Alphavirus; Sindbis virus), Enteroviridae (e.g., Echovirus). Illustrative modified vaccinia viral vectors of use for expressing the present fusion polypeptides are described, e.g., in WO 2019/134049.

In some embodiments, the viral expression vector is an arenavirus vector selected from Lymphocytic choriomeningitis mammarenavirus (LCMV)(NCBI:txid11623), Cali mammarenavirus (a.k.a., Pichinde mammarenavirus or Pichinde arenavirus) (NCBI:txid2169993), Guanarito virus (GTOV) (NCBI:txid45219), Argentinian mammarenavirus (a.k.a., Junin virus (JUNV))(NCBI:txid2169991), Lassa virus (LASV)(NCBI:txid11620), Lujo virus (LUJV)(NCBI:txid649188), Machupo virus (MACV)(NCBI:txid11628), Brazilian mammarenavirus (a.k.a., Sabia virus (SABV))(NCBI:txid2169992), and Whitewater Arroyo virus (WWAV)(NCBI:txid46919). In some embodiments, the viral expression vector is an arenavirus vector selected from Lymphocytic choriomeningitis mammarenavirus (LCMV) or Cali mammarenavirus (a.k.a., Pichinde mammarenavirus or Pichinde arenavirus). Illustrative arenavirus vectors that can be used as delivery and expression vehicles for the herein described fusion polypeptides are described, e.g., in WO 2009/083210; WO 2015/183895; WO 2016/075250; WO 2017/198726; and U.S. Pat. No. 9,943,585.

In some embodiments, the viral expression vector is an adenovirus vector, e.g., from a human adenovirus or a simian adenovirus (e.g., a chimpanzee adenovirus, a gorilla adenovirus or a rhesus monkey adenovirus). In various embodiments, the adenovirus vector is selected from adenovirus serotype 5 (Ad5), adenovirus serotype 26 (Ad26), adenovirus serotype 34 (Ad34), adenovirus serotype 35 (Ad35), adenovirus serotype 48 (Ad48), chimpanzee adenovirus (e.g. ChAd3 (AdC3), ChAd5 (AdC5), ChAd6 (AdC6), ChAd7 (AdC7), ChAd8 (AdC8), ChAd9 (AdC9), ChAd10 (AdC10), ChAd11 (AdC11), ChAd17 (AdC17), ChAd16 (AdC16), ChAd19 (AdC19), ChAd20 (AdC20), ChAd22 (AdC22), ChAd24 (AdC24), ChAdY25, ChAd26 (AdC26), ChAd28 (AdC28), ChAd30 (AdC30), ChAd31 (AdC31), ChAd37 (AdC37), ChAd38 (AdC38), ChAd43 (AdC43), ChAd44 (AdC44), ChAd55 (AdC55), ChAd63 (AdC63), ChAdV63, ChAd68 (AdC68), ChAd73 (AdC73), ChAd82 (AdC82), ChAd83 (AdC83), ChAd143 (AdC143), ChAd144 (AdC144), ChAd145 (AdC145), ChAd147 (AdC147)), gorilla adenovirus (e.g. GC44, GC45, GC46) and rhesus adenovirus (e.g., RhAd51, RhAd52, RhAd53, RhAd54, RhAd55, RhAd56, RhAd57, RhAd58, RhAd59, RhAd60, RhAd61, RhAd62, RhAd63, RhAd64, RhAd65, RhAd66). Illustrative Chimpanzee, Gorilla and Rhesus monkey adenovirus vectors that can be used as delivery and expression vehicles for the herein described fusion polypeptides are described, e.g., in WO 2019/076880; WO 2019/076877; Andrabi et al., (2019) Cell Reports 27:2426-2441 Guo, et al., Hum Vaccin Immunother. (2018) 14(7):1679-1685; Abbink, et al., J Virol. (2015) 89(3):1512-22; and Abbink, et al., J Virol. (2018) 92(6). pii: e01924-17.

In various embodiments, the viral expression vector is incapable of replication (i.e., replication defective or replication deficient), has reduced or diminished capacity for replication, e.g., in comparison to a wild-type viral vector (i.e., replication attenuated) or is replication competent.

In various embodiments, the viral vector or viral expression vector is an adenoviral vector comprising one or more polynucleotides that encode one or more fusion proteins comprising an amino acid sequence of any one of any one of SEQ ID NOs: 345-377, 407-411, 422-424, 430-435, or that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 345-377, 407-411, 422-424, 430-435.

In various embodiments, the viral vector or viral expression vector comprises two or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the following amino acid sequences: SEQ ID NOs: 345 and 346; SEQ ID NOs: 347 and 348; SEQ ID NOs: 349 and 350; SEQ ID NOs: 351 and 352; SEQ ID NOs: 430 and 352; SEQ ID NOs: 357 and 358; SEQ ID NOs: 360 and 362; SEQ ID NOs: 359 and 361; SEQ ID NOs: 351 and 357; SEQ ID NOs: 351 and 358; SEQ ID NOs: 351 and 359; SEQ ID NOs: 351 and 360; SEQ ID NOs: 351 and 361; SEQ ID NOs: 351 and 362; SEQ ID NOs: 351 and 407; SEQ ID NOs: 351 and 408; SEQ ID NOs: 351 and 409; SEQ ID NOs: 351 and 410; SEQ ID NOs: 352 and 357; SEQ ID NOs: 352 and 358; SEQ ID NOs: 352 and 359; SEQ ID NOs: 352 and 360; SEQ ID NOs: 352 and 361; SEQ ID NOs: 352 and 362; SEQ ID NOs: 352 and 407; SEQ ID NOs: 352 and 408; SEQ ID NOs: 352 and 409; SEQ ID NOs: 352 and 410; SEQ ID NOs: 430 and 357; SEQ ID NOs: 430 and 358; SEQ ID NOs: 430 and 359; SEQ ID NOs: 430 and 360; SEQ ID NOs: 430 and 361; SEQ ID NOs: 430 and 362; SEQ ID NOs: 407 and 409; SEQ ID NOs: 407 and 408; SEQ ID NOs: 408 and 410; or SEQ ID NOs: 409 and 410.

In some embodiments, the vector further comprises a polynucleotide encoding a cytokine or functional variant thereof, or a non-coding immunostimulatory polynucleotide. In some embodiments, the vector further comprises a polynucleotide encoding a cytokine selected from the group consisting of IL-2, IL-7, IL-12, IL-15, IL-18, IL-21, IFN-α, IFN-γ, colony stimulating factor 2 (CSF2; a.k.a., GM-CSF), fins related receptor tyrosine kinase 3 ligand (FLT3LG), and combinations and functional variants thereof. Co-expression and/or co-administration of a cytokine with a vaccine is described, e.g., by Elizaga, et al. (2018) PLoS One 13(9): e0202753 (IL-12); Buchbinder, et al., (2017) PLoS One 12(7):e0179597 (GM-CSF); Abaitua, et al., Virus Res (2006) 116(1-2):11-20 (IL12+IFN-γ); Oudard, et al., Cancer Immunol Immunother (2011) February; 60(2):261-71 (IL-2+IFN-α). In some embodiments, the vector further comprises a non-coding immunostimulatory polynucleotide selected from a pathogen-activated molecular pattern (PAMP), a cytosine-phosphate-guanosine (CpG) oligodeoxynucleotide, and an immunostimulatory RNA (isRNA). Illustrative isRNA include CV8102 (CureVac) and others, described in e.g., WO2016170176.

Further provided are host cells comprising one or more polynucleotides encoding one or more of the fusion polypeptides or one or more vectors expressing the fusion polypeptides, as described herein. Any of a variety of host cells can be used. In one embodiment, a host cell is a prokaryotic cell, for example, E. coli. In another embodiment, a host cell is a eukaryotic cell, for example, a yeast cell, a plant cell, an insect cell, a mammalian cell, such as a Chinese Hamster Ovary (CHO)-based or CHO-origin cell line (e.g., CHO-S, CHO DG44, ExpiCHO™, CHOZN® ZFN-modified GS−/− CHO cell line, CHO-K1, CHO-K1a), COS cells, BHK cells, NSO cells or Bowes melanoma cells. Examples of human host cells are, inter alia, HeLa, 911, AT1080, A549 and HEK293 (e.g., HEK293E, HEK293F, HEK293H, HEK293T, Expi293™). In addition, the fusion polypeptides can be expressed in a yeast cell such as Pichia (see, e.g., Powers et al., J Immunol Methods. 251:123-35 (2001)), Hanseula, or Saccharomyces.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As appropriate, the host cells can be stably or transiently transfected with one or more polynucleotides encoding one or more fusion polypeptides, as described herein. As appropriate, the host cells can be infected with one or more vectors expressing one or more fusion polypeptides, as described herein. In some embodiments, the host cells are capable of being infected with and propagating one or more replication attenuated or replication competent vectors expressing one or more fusion polypeptides, as described herein. Illustrative cells useful for infecting with and/or propagating viral vectors include without limitation BHK-21, A549, Vero and HEK293 (e.g., HEK293E, HEK293F, HEK293H, HEK293T, Expi293™) cells. In certain embodiments, the host cells express the Coxsackievirus and adenovirus receptor (CAR), e.g., MDCK, Caco-2 or Calu-3 host cells. In certain embodiments, the polynucleotides integrate into the genome of the host cell.

### 5. Pharmaceutical Compositions/Immunogenic Compositions

Provided are pharmaceutical compositions or immunogenic compositions comprising one or more of the fusion polypeptides, as described herein, or a polynucleotide encoding one or more of the fusion polypeptides, as described herein, or a viral expression vector comprising one or more of such polynucleotides, and a pharmaceutically acceptable diluent, carrier or excipient. Generally, the pharmaceutical compositions described herein are immunogenic. In certain embodiments, the pharmaceutical composition comprises a therapeutically effective amount of the one or more fusion polypeptides, or one or more polynucleotides encoding one or more of the fusion polypeptides, or one or more viral expression vectors containing one or more of the polynucleotides encoding one or more of the fusion polypeptides.

Various pharmaceutically acceptable diluents, carriers, and excipients, and techniques for the preparation and use of pharmaceutical compositions will be known to those of skill in the art in light of the present disclosure. Illustrative pharmaceutical compositions and pharmaceutically acceptable diluents, carriers, and excipients are also described in, e.g., Loyd V. Allen Jr (Editor), “Remington: The Science and Practice of Pharmacy,” 22^{nd} Edition, 2012, Pharmaceutical Press; Brunton, Knollman and Hilal-Dandan, “Goodman and Gilman's The Pharmacological Basis of Therapeutics,” 13th Edition, 2017, McGraw-Hill Education/Medical; McNally and Hastedt (Editors), “Protein Formulation and Delivery, 2nd Edition, 2007, CRC Press; Banga, “Therapeutic Peptides and Proteins: Formulation, Processing, and Delivery Systems,” 3rd Edition, 2015, CRC Press; Lars Hovgaard, Frokjaer and van de Weert (Editors), “Pharmaceutical Formulation Development of Peptides and Proteins,” 2nd Edition, 2012, CRC Press; Carpenter and Manning (Editors), “Rational Design of Stable Protein Formulations: Theory and Practice,” 2002, Springer (Pharmaceutical Biotechnology (Book 13)); Meyer (Editor), “Therapeutic Protein Drug Products: Practical Approaches to Formulation in the Laboratory, Manufacturing, and the Clinic, 2012, Woodhead Publishing.

In certain embodiments, the polynucleotides or vectors are formulated into lipid nanoparticles. For example, in some embodiments where the fusion polypeptides are expressed from self-replicating or self-amplifying RNA molecules, the self-replicating or self-amplifying RNA can be formulated into lipoplexes, such as lipid nanoparticles (LNPs). As used herein, a “lipoplex” refers to cationic liposomes that are nonviral (synthetic) lipid carriers of DNA. As used herein, the term “lipid nanoparticle” refers to one or more spherical nanoparticles with an average diameter of between about 10 to about 1000 nanometers, and which comprise a solid lipid core matrix that can solubilize lipophilic molecules. In certain embodiments, the lipid core is stabilized by surfactants (e.g., emulsifiers), and can comprise one or more of triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate), including combinations thereof. Lipid nanoparticles are described, for example, in Petrilli et al., Curr Pharm Biotechnol. 15:847-55, 2014; and U.S. Pat. Nos. 6,217,912; 6,881,421; 7,402,573; 7,404,969; 7,550,441; 7,727,969; 8,003,621; 8,691,750; 8,871,509; 9,017,726; 9,173,853; 9,220,779; 9,227,917; and 9,278,130, each of which is incorporated by reference in its entirety. In one embodiment, a self-replicating or self-amplifying RNA molecule encoding one or more of the fusion polypeptides described herein is formulated or condensed into polyethylenimine (PEI)-polyplex delivery vehicles, e.g., as described in Demoulins, et al., Nanomedicine. (2016) April; 12(3):711-722 and Demoulins, et al., J Control Release. (2017) Nov. 28; 266:256-271, which can be nanoparticulate.

In embodiments where the fusion polypeptides are expressed from a viral expression vector, the viral expression vector can be formulated for the desired route of administration, e.g., as an isotonic pharmaceutically acceptable aqueous solution for intravenous, intramuscular, subcutaneous or intradermal administration. In some embodiments, the viral expression vector can be formulated for mucosal, e.g., buccal, intranasal or intrarectal delivery. Illustrative formulations for viral expression vectors that can be used in the herein described pharmaceutical compositions and methods are described, e.g., in Manfredsson and Benskey, editors, “Viral Vectors for Gene Therapy: Methods and Protocols (Methods in Molecular Biology),” 2019, Book 1937 in Methods in Molecular Biology Series, Humana Press; WO 2017/013169 (formulation of Adenoviral vectors in an aqueous mixture or freeze dried composition in the presence of amorphous sugar and low salt concentration); and Kumru, et al., J Pharm Sci. (2018) November; 107(11):2764-2774 (aqueous formulations buffered in Tris and containing proline, lactose, and mannitol as stabilizing additives). Formulation of arenavirus vectors is described, e.g., in WO 2009/083210; WO 2016/075250 and WO 2017/198726. In certain embodiments, the viral expression vectors are delivered via microneedle-mediated delivery, e.g., as described in Zaric, et al., Expert Opin Drug Deliv. (2017) October; 14(10):1177-1187. Intranasal viral vaccination by administration of viral particles to the nares is described, e.g., in Dorta-Estremera, et al., PLoS One. 2017 Dec. 8; 12(12):e0188807. Intrarectal viral vaccination by administration of viral particles to the rectum is described, e.g., in Patterson, et al., Clin Vaccine Immunol. (2012) May; 19(5):629-37.

In some embodiments, each carrier, diluent or excipient is “acceptable” in the sense of being compatible with the other ingredients of the pharmaceutical composition and not injurious to the subject. Often, the pharmaceutically acceptable carrier is an aqueous pH-buffered solution. Some examples of materials which can serve as pharmaceutically-acceptable carriers, diluents or excipients include: water; buffers, e.g., a buffer having a pKa in the range of about 6.0 to about 8.0, e.g., a physiologically acceptable buffer, e.g., selected from phosphate, carbonate, bicarbonate, citrate, maleate, glycine-glycine, HEPES, HEPPSO, HEPPS, imidazole, BICINE, TRICINE, Tris, and BIS-Tris; sugars, such as lactose, trehalose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Hank's solution, Ringer's solution; ethyl alcohol; phosphate buffer solutions; amino acids (e.g., charged amino acids, including without limitation, aspartate, asparagine, glutamate, glutamine, histidine, arginine, lysine); and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Solid and semi-solid formulations that can be used for intravaginal or intrarectal (e.g., in the form of a troche, a pessary or a suppository) delivery of viral expression vectors, virosomes or virus-like particles (VLPs) is described, e.g., in Brown, et al., PLoS One. 2017 Aug. 17; 12(8):e0183510; Brown, et al., PLoS One. 2016 Mar. 10; 11(3):e0151184; and Amacker, et al., npj Vaccines 5, 41 (2020).

In one particular formulation, an arenavirus vector (e.g., a LCMV or Pichinde mammarenavirus vector) described herein is formulated in an isotonic aqueous solution comprising a biologically compatible buffer having a pKa in the range of about 6.0 to about 8.0 (e.g., HEPES and NaCl), at a neutral or near-neutral pH and a non-ionic surfactant (e.g., PLURONIC® F68 (a.k.a., poloxamer 188)). In one particular formulation, an arenavirus vector (e.g., a LCMV or Pichinde mammarenavirus vector) described herein is formulated in an isotonic aqueous solution comprising HEPES buffer at pH 7.4, NaCl, and PLURONIC® F68 (a.k.a., poloxamer 188). Schleiss, et al. (Clin Vaccine Immunol. 2017 Jan. 5; 24(1):e00300-16) describes an LCMV formulating LCMV vectors in a diluent of 25 mM HEPES, 150 mM NaCl, 0.01% PLURONIC® F68; pH 7.4), which can be used to formulate the herein described arenavirus vectors. A final concentration of 10% sorbitol was added before freezing below −60° C.

The formulation of and delivery methods of pharmaceutical compositions will generally be adapted according to the site and the disease to be treated. Exemplary formulations include without limitation, those suitable for parenteral administration, e.g., intravenous, intra-arterial, intramuscular, or subcutaneous administration, including formulations encapsulated in micelles, liposomes or drug-release capsules (active agents incorporated within a biocompatible coating designed for slow-release); ingestible formulations; formulations for topical use, such as creams, ointments and gels; and other formulations such as inhalants, aerosols and sprays. In some embodiments, the pharmaceutical compositions are formulated for parenteral, e.g., intravenous, subcutaneous, or oral administration. In some embodiments, the pharmaceutical compositions are formulated for mucosal, e.g., buccal, intranasal, intrarectal and/or intravaginal administration.

In certain embodiments, pharmaceutical compositions are sterile. In certain embodiments, the pharmaceutical composition has a pH in the range of 4.5 to 8.5, 4.5 to 6.5, 6.5 to 8.5, or a pH of about 5.0, about 5.5, about 6.0, about 6.5, about 7.0, about 7.5, about 8.0 or about 8.5. In one embodiment, the pharmaceutical composition has an osmolarity in the range of 240-260 or 250-330 mOsmol/L. In certain embodiments, the pharmaceutical composition is isotonic or near isotonic.

In some embodiments, the pharmaceutical compositions are liquids or solids. In some embodiments, the pharmaceutical composition comprises an aqueous solution. In some embodiments, the pharmaceutical composition is lyophilized or is a frozen liquid.

In some embodiments, the pharmaceutical composition further comprises one or more additional therapeutic agents, e.g., a second therapeutic agent, or second and third therapeutic agents, for use in combination therapies, as described herein.

In certain embodiments, the pharmaceutical composition further comprises an adjuvant. Illustrative adjuvants that can be co-formulated or co-administered with the herein described fusion polypeptides, polynucleotides encoding such fusion polypeptides and vectors expressing such fusion polypeptides include without limitation cytokines, chemokines, immune costimulatory molecules, toll-like receptor agonists or inhibitors of immune suppressive pathways, as described herein, and in Li, et al., Curr Issues Mol Biol. (2017) 22:17-40. Other adjuvants that can be co-formulated or co-administered with the herein described fusion polypeptides, polynucleotides encoding such fusion polypeptides and vectors expressing such fusion polypeptides include without limitation mineral salts (e.g., aluminum salts (e.g., alum), calcium phosphate, incomplete Freunds's adjuvant), lipid particles (e.g., MF59, cochleates, virus-like particles), microparticles (e.g., virosomes, polylactic acid (PLA), poly[lactide-coglycolide] (PLG)), immune potentiators (e.g., dsRNA:Poly(I:C), Poly-IC:LC, Monophosphoryl lipid A (MPL), LPS, Flagellin, Imidazoquinolines: imiquimod (R837), resiquimod (848), CpG oligodeoxynucleotides (ODN), Muramyl dipeptide (MDP), Saponins (QS-21)), and mucosal adjuvants (e.g., Cholera toxin (CT), Heat-labile enterotoxin (LTK3 and LTR72), Chitosan). Adjuvants that can be co-formulated or co-administered with the herein described fusion polypeptides, polynucleotides encoding such fusion polypeptides and vectors expressing such fusion polypeptides are summarized in Apostolico, et al., J Immunol Res. (2016) 2016:1459394.

In certain embodiments, the pharmaceutical composition further comprises an immunomodulator. Illustrative immunomodulators that can be co-formulated or co-administered with the herein described fusion polypeptides, polynucleotides encoding such fusion polypeptides and vectors expressing such fusion polypeptides include without limitation toll-like receptor agonists and small molecule immune checkpoint inhibitors. Example TLR7 agonists that can be co-formulated or co-administered include without limitation AL-034, DSP-0509, GS-9620 (vesatolimod), LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854 and RG-7795. Illustrative TLR7/TLR8 agonists that can be co-formulated or co-administered include CV8102, NKTR-262, telratolimod and BDB-001. Example TLR8 agonists that can be co-formulated or co-administered include without limitation E-6887, IMO-4200, IMO-8400, IMO-9200, MCT-465, MEDI-9197, motolimod, resiquimod, GS-9688, VTX-1463, VTX-763, 3M-051, 3M-052. Example TLR9 agonists that can be co-formulated or co-administered include without limitation AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of small molecule inhibitors of CD274 or PDCD1 that can be co-formulated or co-administered include without limitation GS-4224, GS-4416, INCB086550 and MAX10181. An example small molecule inhibitor of CTLA4 that can be co-formulated or co-administered includes BPI-002.

In some embodiments, the pharmaceutical compositions or immunogenic compositions comprise mixtures of two or more fusion polypeptides, two or more polynucleotides encoding such fusion polypeptides, or two or more vectors expressing such fusion polypeptides. For example, in certain embodiments, the mixtures comprise bivalent pairs of fusion polypeptides, as described herein. In some embodiments, the pharmaceutical composition comprises two or more fusion polypeptides, two or more polynucleotides encoding such fusion polypeptides, or two or more vectors expressing such fusion polypeptides, the fusion polypeptides comprising or consisting of the following polypeptide segments in sequential order, from N-terminus to C-terminus, optionally joined or connected by one or more linkers: SEQ ID NOs: 70, 76, 94, 151 and 161; and SEQ ID NOs: 71, 77, 95, 152 and 162. In some embodiments, the pharmaceutical composition comprises two or more fusion polypeptides, two or more polynucleotides encoding such fusion polypeptides, or two or more vectors expressing such fusion polypeptides, the fusion polypeptides comprising or consisting of the following polypeptide segments in sequential order, from N-terminus to C-terminus, optionally joined or connected by one or more linkers: SEQ ID NOs: 188, 305, 28, 41, 294, 4, 176, 11, 319, 259, 282, 223, 213 and 37; SEQ ID NOs: 188, 305, 28, 41 and 294; SEQ ID NOs: 4, 176, 11, 319, 259, 282, 223, 213 and 37; SEQ ID NOs: 189, 306, 29, 42, 295, 5, 177, 12, 320, 260, 283, 224, 214 and 38; SEQ ID NOs: 189, 306, 29, 42 and 295; SEQ ID NOs: 5, 177, 12, 320, 260, 283, 224, 214 and 38; SEQ ID NOs: 305, 319, 259, 282, 223, 213, 294, 176 and 188; SEQ ID NOs: 306, 320, 260, 283, 224, 214, 295, 177 and 189; SEQ ID NOs: 305, 294, 223, 213, 176, 259, 319, 188 and 282; SEQ ID NOs: 306, 295, 224, 214, 177, 260, 320, 189 and 283; SEQ ID NOs: 305, 294, 319, 259, 282, 223, 176, and 188; SEQ ID NOs: 306, 295, 320, 260, 283, 224, 177 and 189; SEQ ID NOs: 305, 223, 294, 176, 259, 319, 188 and 282; and SEQ ID NOs: 306, 224, 295, 177, 260, 320, 189 and 283. In some embodiments, the pharmaceutical composition comprises two or more fusion polypeptides, two or more polynucleotides encoding such fusion polypeptides, or two or more vectors expressing such fusion polypeptides, the fusion polypeptides comprising or consisting of the following polypeptide segments in sequential order, from N-terminus to C-terminus, optionally joined or connected by one or more linkers: SEQ ID NOs: 76, 86, 94, 180, 186, 221, 294, 307, 321 and 151; and SEQ ID NOs: 77, 87, 95, 181, 187, 222, 295, 308, 322 and 152.

In some embodiments, the pharmaceutical composition or immunogenic composition comprises two or more fusion polypeptides, two or more polynucleotides encoding such fusion polypeptides, or two or more vectors expressing such fusion polypeptides, the fusion polypeptides comprising or consisting of an amino acid sequence of any one of SEQ ID NOs: 351-356 and 430, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 351-356 and 430. In some embodiments, the pharmaceutical composition or immunogenic composition comprises two or more fusion polypeptides, two or more polynucleotides encoding such fusion polypeptides, or two or more vectors expressing such fusion polypeptides, the fusion polypeptides comprising or consisting of an amino acid sequence of any one of SEQ ID NOs: 357-366 and 407-410, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 357-366 and 407-410.

In some embodiments, the pharmaceutical composition or immunogenic composition comprises two or more fusion polypeptides, two or more polynucleotides encoding such fusion polypeptides, or two or more vectors expressing such fusion polypeptides, the fusion polypeptides comprising or consisting of an amino acid sequence of any one of SEQ ID NOs: 345-350, the sequences in Table 1, and SEQ ID NOs: 422-424, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NO: 345-350, the sequences in Table 1, and SEQ ID NOs: 422-424.

In some embodiments, the pharmaceutical compositions or immunogenic compositions comprise a first fusion polypeptide or polynucleotide encoding such fusion polypeptide or a vector expressing such fusion polypeptide, the fusion polypeptide comprising one or more polypeptide segments encoded by HIV-1 Gag and Nef genes and a second fusion polypeptide or polynucleotide encoding such fusion polypeptide or viral expression vector expressing such fusion polypeptide, the fusion polypeptide comprising one or more polypeptide segments encoded by HIV-1 Pol or Pol and Env genes. In some embodiments, the pharmaceutical composition or immunogenic composition comprises (1) one or more fusion polypeptides or one or more polynucleotides encoding such fusion polypeptides or one or more vectors expressing such fusion polypeptides, the fusion polypeptide comprising or consisting of the following polypeptide segments in sequential order, from N-terminus to C-terminus, optionally joined or connected by one or more linkers: SEQ ID NOs: 70, 76, 94, 151 and 161; or SEQ ID NOs: 71, 77, 95, 152 and 162; and (2) one or more fusion polypeptides or one or more polynucleotides encoding such fusion polypeptides or one or more vectors expressing such fusion polypeptides, the fusion polypeptide comprising or consisting of the following polypeptide segments in sequential order, from N-terminus to C-terminus, optionally joined or connected by one or more linkers: SEQ ID NOs: 188, 305, 28, 41, 294, 4, 176, 11, 319, 259, 282, 223, 213 and 37; SEQ ID NOs: 188, 305, 28, 41 and 294; SEQ ID NOs: 4, 176, 11, 319, 259, 282, 223, 213 and 37; SEQ ID NOs: 189, 306, 29, 42, 295, 5, 177, 12, 320, 260, 283, 224, 214 and 38; SEQ ID NOs: 189, 306, 29, 42 and 295; SEQ ID NOs: 5, 177, 12, 320, 260, 283, 224, 214 and 38; SEQ ID NOs: 305, 319, 259, 282, 223, 213, 294, 176 and 188; SEQ ID NOs: 306, 320, 260, 283, 224, 214, 295, 177 and 189; SEQ ID NOs: 305, 294, 223, 213, 176, 259, 319, 188 and 282; SEQ ID NOs: 306, 295, 224, 214, 177, 260, 320, 189 and 283; SEQ ID NOs: 305, 294, 319, 259, 282, 223, 176, and 188; SEQ ID NOs: 306, 295, 320, 260, 283, 224, 177 and 189; SEQ ID NOs: 305, 223, 294, 176, 259, 319, 188 and 282; or SEQ ID NOs: 306, 224, 295, 177, 260, 320, 189 and 283. In some embodiments, the pharmaceutical composition or immunogenic composition comprises (1) one or more fusion polypeptides or one or more polynucleotides encoding such fusion polypeptides or one or more vectors expressing such fusion polypeptides, the fusion polypeptide comprising an amino acid sequence of any one of SEQ ID NOs: 351-356 and 430, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 351-356 and 430; and (2) one or more fusion polypeptides or one or more polynucleotides encoding such fusion polypeptides or one or more vectors expressing such fusion polypeptides, the fusion polypeptide comprising an amino acid sequence of any one of SEQ ID NOs: 357-366 and 407-410, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 357-366 and 407-410.

In some embodiments, the pharmaceutical composition or immunogenic composition comprises one or more viral vectors, each viral vector comprising one or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the following amino acid sequences: SEQ ID NOs: 345 and 346; SEQ ID NOs: 347 and 348; SEQ ID NOs: 349 and 350; SEQ ID NOs: 351 and 352; SEQ ID NOs: 430 and 352; SEQ ID NOs: 357 and 358; SEQ ID NOs: 360 and 362; SEQ ID NOs: 359 and 361; SEQ ID NOs: 351 and 357; SEQ ID NOs: 351 and 358; SEQ ID NOs: 351 and 359; SEQ ID NOs: 351 and 360; SEQ ID NOs: 351 and 361; SEQ ID NOs: 351 and 362; SEQ ID NOs: 351 and 407; SEQ ID NOs: 351 and 408; SEQ ID NOs: 351 and 409; SEQ ID NOs: 351 and 410; SEQ ID NOs: 352 and 357; SEQ ID NOs: 352 and 358; SEQ ID NOs: 352 and 359; SEQ ID NOs: 352 and 360; SEQ ID NOs: 352 and 361; SEQ ID NOs: 352 and 362; SEQ ID NOs: 352 and 407; SEQ ID NOs: 352 and 408; SEQ ID NOs: 352 and 409; SEQ ID NOs: 352 and 410; SEQ ID NOs: 430 and 357; SEQ ID NOs: 430 and 358; SEQ ID NOs: 430 and 359; SEQ ID NOs: 430 and 360; SEQ ID NOs: 430 and 361; SEQ ID NOs: 430 and 362; SEQ ID NOs: 407 and 409; SEQ ID NOs: 407 and 408; SEQ ID NOs: 408 and 410; or SEQ ID NOs: 409 and 410.

In some embodiments, the pharmaceutical composition or immunogenic composition comprises a fusion polypeptide, a polynucleotide encoding such polypeptide or a vector expressing such fusion polypeptide, the fusion polypeptide comprising or consisting of the following polypeptide segments in sequential order, from N-terminus to C-terminus, optionally joined or connected by one or more linkers: SEQ ID NOs: 201, 78, 107, 96, 229, 172, 327, 6, 333, 243, 331, 192, 265, 311, 137, 15, 123, 30, 336, 302, 153, 219, 298, 121, 230, 240, 60, 241, 276, 113, 99, 21, 217 and 215; SEQ ID NOs: 78, 296, 1, 339, 197, 329, 232, 323, 303, 234, 90, 261, 274, 238, 211, 325, 137, 227, 209, 190, 341, 57, 225, 27, 210, 119, 19, 165, 334, 117, 153, 10, 97 and 300; or SEQ ID NOs: 296, 1, 78, 197, 339, 227, 261, 274, 238, 325, 137, 329, 303, 234, 90, 232, 27, 57, 225, 323, 190, 341, 119, 19, 165, 334, 117, 153, 10, 97 and 300. In some embodiments, the pharmaceutical composition or immunogenic composition comprises a fusion polypeptide, a polynucleotide encoding such polypeptide or a vector expressing such fusion polypeptide, the fusion polypeptide comprising or consisting of an amino acid sequence of any one of SEQ ID NOs: 367-377, 411, 422-424 and 431-435, or a sequence that is 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 367-377, 411, 422-424 and 431-435.

### 6. Methods of Treatment

Further provided are methods for treating or preventing an HIV infection or a related disease or disorder in a subject in need thereof (e.g., a human subject), comprising providing to a subject in need thereof an effective amount of one or more fusion polypeptides, as described herein, or one or more polynucleotides encoding one or more fusion polypeptides, as described herein, or one or more vectors expressing one or more fusion polypeptides, as described herein. As used herein, the term “subject” refers to a mammal. The mammal can be any mammal, for example, a human, a non-human primate (e.g., a macaque), a rodent (e.g., mouse, rat, guinea pig), a dog, a cat, or a domesticated animal such as a cow, a horse, a goat, a camel, a sheep or a pig. The term “patient” refers to a human subject. As used herein, the term “effective amount” in the context of the administration of a therapy to a subject refers to the amount of a therapy that achieves a desired prophylactic or therapeutic effect. The polynucleotide may be present in a vector, e.g., a viral vector, as described herein. In some embodiments, the related disease or disorder is caused by infection with HIV. In other embodiments, it is acquired immune deficiency syndrome (AIDS). In certain embodiments, the subject is a virologically suppressed HIV-infected mammal, while in other embodiments, the subject is a treatment-naïve HIV-infected mammal or a treatment experienced HIV-infected subject that is not virologically suppressed. In certain embodiments, a treatment-naïve subject has a viral load between <50 copies/mL and 10⁸ copies/ml. In certain embodiments, a virologically suppressed subject has a viral load <50 copies/ml. In another embodiment, the subject is a mammal, e.g., a human. In certain embodiments, the subject has been diagnosed with an HIV, e.g., HIV-1 or HIV-2, infection or a related disease or disorder, e.g., AIDS, or is considered at risk for developing an HIV, e.g., HIV-1 or HIV-2, infection or a related disease or disorder, e.g., AIDS. Subjects at risk for HIV-related diseases or disorders include patients who have come into contact with an infected person or who have been exposed to HIV in some other way. Administration of a prophylactic agent can occur prior to the manifestation of symptoms characteristic of HIV-related disease or disorder, such that a disease or disorder is prevented or, alternatively, delayed in its progression.

In some embodiments, the subject is chronically infected with HIV-1. In some embodiments, the subject is acutely infected with HIV-1, e.g., has an HIV-1 infection of Fiebig stage IV or earlier, e.g. Fiebig stage III, Fiebig stage II or Fiebig stage I. In some embodiments, the subject is not receiving antiretroviral therapy (ART) or ART is discontinued prior to administration of the one or more compositions. In some embodiments, ART is discontinued after one or more administrations of the compositions. In some embodiments, ART is administered concurrently with administration of one or more fusion polypeptides, as described herein, or one or more polynucleotides encoding one or more fusion polypeptides, as described herein, or one or more vectors expressing one or more fusion polypeptides, as described herein.

Also provided are methods for preventing or inhibiting an increase in HIV virus titer, virus replication, virus proliferation or an amount of an HIV viral DNA, HIV proviral DNA, or HIV viral protein in a subject (e.g., a human subject). In one embodiment, the method comprises providing to the subject in need thereof an amount of an one or more fusion polypeptides, as described herein, or one or more polynucleotides encoding one or more fusion polypeptides, as described herein, or one or more vectors expressing one or more fusion polypeptides, as described herein, effective to prevent an increase in HIV titer, virus replication, or an amount of an HIV protein of one or more HIV strains or isolates in the subject. In certain embodiments, the method further comprises measuring an amount of HIV viral or proviral DNA or protein at one or more time points, e.g., before and after the subject in provided with one or more fusion polypeptides, as described herein, or one or more polynucleotides encoding one or more fusion polypeptides, as described herein, or one or more vectors expressing one or more fusion polypeptides, as described herein. Methods and biomarkers for determining an amount of HIV viral or proviral DNA or protein in a subject are known and available in the art, and described for example, in Siliciano, J. D. et al., Curr Opin. HIV AIDS, 5(6):491-7 (2010), and Rouzioux, C. et al., Curr Opin HIV AIDS, 8(3):170-5 (2013).

In some embodiments, one or more fusion polypeptides, as described herein, or one or more polynucleotides encoding one or more fusion polypeptides, as described herein, or one or more vectors expressing one or more fusion polypeptides, as described herein, may be used in, for example, methods of inhibiting certain viruses such as HIV isolates described herein, prophylactic inhibiting or preventing infections of certain viruses such as HIV isolates described herein, detection of certain viruses such as HIV isolates described herein in a sample, inhibiting certain viruses such as HIV isolates described herein, or diagnosis of certain viruses such as HIV isolates described herein.

For in vivo treatment of mammalian subject, e.g., humans, the subject may be administered or provided a pharmaceutical composition comprising one or more fusion polypeptides, as described herein, or one or more polynucleotides encoding one or more fusion polypeptides, as described herein, or one or more vectors expressing one or more fusion polypeptides, as described herein. When used for in vivo therapy, the one or more fusion polypeptides, as described herein, or one or more polynucleotides encoding one or more fusion polypeptides, as described herein, or one or more vectors expressing one or more fusion polypeptides, as described herein, are typically administered or provided to the patient in therapeutically effective amounts (i.e., amounts that eliminate or reduce the patient's viral burden and/or viral reservoir). The one or more fusion polypeptides, as described herein, or one or more polynucleotides encoding one or more fusion polypeptides, as described herein, or one or more vectors expressing one or more fusion polypeptides, as described herein, are administered or provided to a mammalian subject, e.g., a human, in accord with known methods, such as, but not limited to, intravenous administration, e.g., as a bolus or by continuous infusion over a period of time, by intramuscular, intraperitoneal, intracerebrospinal, subcutaneous, intraarticular, intrasynovial, intrathecal, oral, topical, or inhalation routes. The one or more fusion polypeptides, as described herein, or one or more polynucleotides encoding one or more fusion polypeptides, as described herein, or one or more vectors expressing one or more fusion polypeptides, as described herein, may be administered parenterally, when possible, at the target cell site, or intravenously. In one embodiment, administration of the one or more fusion polypeptides, as described herein, or one or more polynucleotides encoding one or more fusion polypeptides, as described herein, or one or more vectors expressing one or more fusion polypeptides, as described herein, to the subject is via an intravenous route. In another embodiment, administration of the one or more fusion polypeptides, as described herein, or one or more polynucleotides encoding one or more fusion polypeptides, as described herein, or one or more vectors expressing one or more fusion polypeptides, as described herein, to the subject is via a subcutaneous route. In additional embodiments, pharmaceutical compositions of the disclosure are administered to a subject systemically, parenterally, or locally (e.g., mucosally, including buccal, intrarectal and/or intravaginal routes).

In certain embodiments, the present disclosure provides a method for treating an HIV infection, comprising administering to a human subject in need thereof a therapeutically effective amount of one or more fusion polypeptides, as described herein, or one or more polynucleotides encoding one or more fusion polypeptides, as described herein, or one or more vectors expressing one or more fusion polypeptides, as described herein. In some embodiments, the present disclosure provides a method for preventing an HIV infection, comprising administering to a human subject in need thereof a therapeutically effective amount of one or more fusion polypeptides, as described herein, or one or more polynucleotides encoding one or more fusion polypeptides, as described herein, or one or more vectors expressing one or more fusion polypeptides, as described herein.

In various embodiments, the methods comprise administering a single fusion polypeptide, or a polynucleotide or viral expression vector encoding the fusion polypeptide, wherein the fusion polypeptide comprises two or more multivalent polypeptide segments, e.g., bivalent polypeptide segments. In some embodiments, two or more fusion polypeptides, or two or more viral expression vectors encoding the fusion polypeptides, are administered to the subject simultaneously or concurrently. In some embodiments, the two or more fusion polypeptides, or two or more polynucleotides or two or more viral expression vectors encoding the fusion polypeptides, are in the form of a bivalent antigen composition.

In some embodiments, the methods entail administering to the subject: (1) one or more fusion polypeptides, or polynucleotides encoding, or viral expression vectors expressing the fusion polypeptides, the fusion polypeptides comprising or consisting of the following polypeptide segments in sequential order, from N-terminus to C-terminus, optionally joined or connected by one or more linkers: SEQ ID NOs: 70, 76, 94, 151 and 161; or SEQ ID NOs: 71, 77, 95, 152 and 162; and (2) one or more fusion polypeptides, or polynucleotides encoding, or viral expression vectors expressing the fusion polypeptides, the fusion polypeptides comprising or consisting of the following polypeptide segments in sequential order, from N-terminus to C-terminus, optionally joined or connected by one or more linkers: SEQ ID NOs: 188, 305, 28, 41, 294, 4, 176, 11, 319, 259, 282, 223, 213 and 37; SEQ ID NOs: 188, 305, 28, 41 and 294; SEQ ID NOs: 4, 176, 11, 319, 259, 282, 223, 213 and 37; SEQ ID NOs: 189, 306, 29, 42, 295, 5, 177, 12, 320, 260, 283, 224, 214 and 38; SEQ ID NOs: 189, 306, 29, 42 and 295; SEQ ID NOs: 5, 177, 12, 320, 260, 283, 224, 214 and 38; SEQ ID NOs: 305, 319, 259, 282, 223, 213, 294, 176 and 188; SEQ ID NOs: 306, 320, 260, 283, 224, 214, 295, 177 and 189; SEQ ID NOs: 305, 294, 223, 213, 176, 259, 319, 188 and 282; SEQ ID NOs: 306, 295, 224, 214, 177, 260, 320, 189 and 283; SEQ ID NOs: 305, 294, 319, 259, 282, 223, 176, and 188; SEQ ID NOs: 306, 295, 320, 260, 283, 224, 177 and 189; SEQ ID NOs: 305, 223, 294, 176, 259, 319, 188 and 282; or SEQ ID NOs: 306, 224, 295, 177, 260, 320, 189 and 283.

In some embodiments, the methods entail administering to the subject: (1) one or more fusion polypeptides, or polynucleotides encoding, or viral expression vectors expressing the fusion polypeptides, the fusion polypeptides comprising or consisting of an amino acid sequence of any one of SEQ ID NOs: 351-356 and 430, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 351-356 and 430; and (2) one or more fusion polypeptides, or polynucleotides encoding, or viral expression vectors expressing the fusion polypeptides, the fusion polypeptides comprising or consisting of an amino acid sequence of any one of SEQ ID NOs: 357-366 and 407-410, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 357-366 and 407-410.

In some embodiments, the method comprises administering to the subject one or more viral vectors, wherein each viral vector comprises two or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the following amino acid sequences: SEQ ID NOs: 345 and 346; SEQ ID NOs: 347 and 348; SEQ ID NOs: 349 and 350; SEQ ID NOs: 351 and 352; SEQ ID NOs: 430 and 352; SEQ ID NOs: 357 and 358; SEQ ID NOs: 360 and 362; SEQ ID NOs: 359 and 361; SEQ ID NOs: 351 and 357; SEQ ID NOs: 351 and 358; SEQ ID NOs: 351 and 359; SEQ ID NOs: 351 and 360; SEQ ID NOs: 351 and 361; SEQ ID NOs: 351 and 362; SEQ ID NOs: 351 and 407; SEQ ID NOs: 351 and 408; SEQ ID NOs: 351 and 409; SEQ ID NOs: 351 and 410; SEQ ID NOs: 352 and 357; SEQ ID NOs: 352 and 358; SEQ ID NOs: 352 and 359; SEQ ID NOs: 352 and 360; SEQ ID NOs: 352 and 361; SEQ ID NOs: 352 and 362; SEQ ID NOs: 352 and 407; SEQ ID NOs: 352 and 408; SEQ ID NOs: 352 and 409; SEQ ID NOs: 352 and 410; SEQ ID NOs: 430 and 357; SEQ ID NOs: 430 and 358; SEQ ID NOs: 430 and 359; SEQ ID NOs: 430 and 360; SEQ ID NOs: 430 and 361; SEQ ID NOs: 430 and 362; SEQ ID NOs: 407 and 409; SEQ ID NOs: 407 and 408; SEQ ID NOs: 408 and 410; or SEQ ID NOs: 409 and 410.

In some embodiments, the methods entail administering to the subject one or more fusion polypeptides, or polynucleotides encoding, or viral expression vectors expressing the fusion polypeptides, the fusion polypeptides comprising or consisting of the following polypeptide segments in sequential order, from N-terminus to C-terminus, optionally joined or connected by one or more linkers: SEQ ID NOs: 201, 78, 107, 96, 229, 172, 327, 6, 333, 243, 331, 192, 265, 311, 137, 15, 123, 30, 336, 302, 153, 219, 298, 121, 230, 240, 60, 241, 276, 113, 99, 21, 217 and 215; SEQ ID NOs: 78, 296, 1, 339, 197, 329, 232, 323, 303, 234, 90, 261, 274, 238, 211, 325, 137, 227, 209, 190, 341, 57, 225, 27, 210, 119, 19, 165, 334, 117, 153, 10, 97 and 300; or SEQ ID NOs: 296, 1, 78, 197, 339, 227, 261, 274, 238, 325, 137, 329, 303, 234, 90, 232, 27, 57, 225, 323, 190, 341, 119, 19, 165, 334, 117, 153, 10, 97 and 300.

In some embodiments, the methods entail administering to the subject one or more fusion polypeptides, or polynucleotides encoding, or viral expression vectors expressing the fusion polypeptides, the fusion polypeptides comprising or consisting of an amino acid sequence of any one of SEQ ID NOs: 367-377, 411, 422-424 and 431-435, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 367-377, 411, 422-424 and 431-435.

In some embodiments, the methods entail administering one or more viral expression vectors that express one or more of the fusion polypeptides. In various embodiments, the methods entail administering from about 10³ to about 10¹² viral focus forming units (FFU) or plaque forming units (PFU) or infectious units (IU) or viral particles (vp), e.g. from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration.

In various embodiments, the methods implement a prime-boost regimen. In various embodiments, the prime-boost regimen comprises administering a priming composition at a first time point and administering one or more boosting compositions at one or more subsequent time points (e.g., prime-boost-boost-boost, etc.). In various embodiments, the prime-boost regimen comprises one or more iterations of administering a priming composition at a first time point and administering a boosting composition at a second time point (e.g., prime-boost-prime-boost, etc.). Implementing a prime-boost regimen comprises one or more iterations of administering a priming composition at a first time point and administering a boosting composition at a second time point (e.g., prime-boost-prime-boost, etc.) can facilitate an immune response predominantly focused or trained on the fusion polypeptides, and reduce or avoid inducing an immune response focused or trained on the vector backbone and/or vector specific proteins. In some embodiments, the administrations of the priming composition and the one or more boosting compositions are spaced at least 1 week, 2 weeks, 3 weeks or 1 month apart, e.g., at least 2, 3, 4, 5 or 6 months, apart. In some embodiments, the priming composition and the boosting composition comprise the same immunogenic composition. In some embodiments, the priming composition and the boosting composition comprise different immunogenic compositions. In some embodiments, the priming composition and the boosting composition comprise the same one or more fusion polypeptides and same polynucleotide or viral expression vector. In some embodiments, the priming composition and the boosting composition comprise different fusion polypeptides and the same polynucleotide or viral expression vectors. In some embodiments, the priming composition and the boosting composition comprise the same fusion polypeptides and different polynucleotide or viral expression vectors. In some embodiments, the methods entail priming with a first polynucleotide or viral expression vector, and boosting with a second polynucleotide or viral expression vector.

In various embodiments, the prime-boost regimen comprises:
a) Priming with a viral expression vector and boosting with a polynucleotide, wherein the polynucleotide is DNA, cDNA, mRNA or self-replicating RNA;
b) Priming with a polynucleotide, wherein the polynucleotide is DNA, cDNA, mRNA or self-replicating RNA, and boosting with a viral expression vector;
c) Priming with a first viral expression vector and boosting with a second viral expression vector, wherein the first and second viral expression vectors are from identical, related or unrelated taxonomical families;
d) Priming with a first replication deficient viral expression vector and boosting with a second replication deficient viral expression vector, wherein the first and second replication deficient viral expression vectors are from identical, related or unrelated taxonomical families;
e) Priming with a first attenuated deficient viral expression vector and boosting with a second replication attenuated viral expression vector, wherein the first and second replication attenuated viral expression vectors are from identical, related or unrelated taxonomical families;
f) Priming with a replication deficient viral expression vector and boosting with a replication attenuated viral expression vector;
g) Priming with a replication attenuated viral expression vector and boosting with a replication deficient viral expression vector;
h) Priming with a Lymphocytic choriomeningitis mammarenavirus (LCMV) viral expression vector and boosting with a Pichinde mammarenavirus viral expression vector;
i) Priming with a Pichinde mammarenavirus viral expression vector and boosting with a Lymphocytic choriomeningitis mammarenavirus (LCMV) viral expression vector;
j) Priming with an arenavirus viral expression vector and boosting with an adenovirus viral expression vector; or
k) Priming with an adenovirus viral expression vector and boosting with an arenavirus viral expression vector.

In some embodiments, after one or more administrations of the one or more fusion polypeptides, as described herein, or one or more polynucleotides encoding one or more fusion polypeptides, as described herein, or one or more vectors expressing one or more fusion polypeptides, as described herein, optionally with one or more additional therapeutic agents, described herein, the subject does not exhibit symptoms of HIV or AIDS in the absence of anti-retroviral treatment (ART) for at least 6 months, at least 1 year, at least 2 years, at least 3 years, or more. In some embodiments, after one or more administrations of the binding molecule, the subject has a viral load of copies/ml blood of less than 500, e.g., less than 400, less than 300, less than 200, less than 100, less than 50, in the absence of anti-retroviral treatment (ART) for at least 6 months, at least 1 year, at least 2 years, at least 3 years, or more.

### 7. Combination Therapies

In certain embodiments, a method for treating or preventing an HIV infection in a human having or at risk of having the infection is provided, comprising administering to the human a therapeutically effective amount of one or more fusion polypeptides, or polynucleotides encoding or vectors expressing such fusion polypeptides, as disclosed herein, in combination with a therapeutically effective amount of one or more (e.g., one, two, three, one or two, or one to three) additional therapeutic agents. In one embodiment, a method for treating an HIV infection in a human having or at risk of having the infection is provided, comprising administering to the human a therapeutically effective amount of a compound disclosed herein, or a pharmaceutically acceptable salt thereof, in combination with a therapeutically effective amount of one or more (e.g., one, two, three, one or two, or one to three) additional therapeutic agents.

In various embodiments, of one or more fusion polypeptides, or polynucleotides encoding or vectors expressing such fusion polypeptides, as disclosed herein, are administered in combination with one or more (e.g., one, two, three, one or two, or one to three) additional therapeutic agents.

In certain embodiments, the provided are methods for treating an HIV infection, comprising administering to a patient in need thereof a therapeutically effective amount of a compound disclosed herein, or a pharmaceutically acceptable salt thereof, in combination with a therapeutically effective amount of one or more (e.g., one, two, three, one or two, or one to three) additional therapeutic agents which are suitable for treating an HIV infection.

In certain embodiments, one or more fusion polypeptides, or polynucleotides encoding or vectors expressing such fusion polypeptides, as disclosed herein, is co-formulated with one, two, three, four, or more additional therapeutic agents, and a pharmaceutically acceptable carrier. In certain embodiments, one or more fusion polypeptides, or polynucleotides encoding or vectors expressing such fusion polypeptides, as disclosed herein, or a pharmaceutically acceptable salt thereof, is combined with two additional therapeutic agents. As appropriate, the one, two, three, four, or more additional therapeutic agents can be different therapeutic agents selected from the same class of therapeutic agents, and/or they can be selected from different classes of therapeutic agents.

### Administration of HIV Combination Therapy

In certain embodiments, a one or more fusion polypeptides, or polynucleotides encoding or vectors expressing such fusion polypeptides, as disclosed herein, are administered with one or more additional therapeutic agents. Co-administration of a compound disclosed herein with one or more additional therapeutic agents generally refers to simultaneous or concurrent, or sequential, administration of a compound disclosed herein and one or more additional therapeutic agents, such that therapeutically effective amounts of the compound disclosed herein and the one or more additional therapeutic agents are both present in the body of the patient. When administered sequentially, the combination may be administered in two or more administrations.

Co-administration includes administration of unit dosages of the compounds disclosed herein before or after administration of unit dosages of one or more additional therapeutic agents. For example, the one or more fusion polypeptides, or polynucleotides encoding or vectors expressing such fusion polypeptides, as disclosed herein, may be administered within seconds, minutes, or hours of the administration of the one or more additional therapeutic agents. In some embodiments, a unit dose of a one or more fusion polypeptides, or polynucleotides encoding or vectors expressing such fusion polypeptides, as disclosed herein, is administered first, followed within seconds or minutes by administration of a unit dose of one or more additional therapeutic agents. Alternatively, a unit dose of one or more additional therapeutic agents is administered first, followed by administration of a unit dose of a one or more fusion polypeptides, or polynucleotides encoding or vectors expressing such fusion polypeptides, as disclosed herein, within seconds or minutes. In other embodiments, a unit dose of one or more fusion polypeptides, or polynucleotides encoding or vectors expressing such fusion polypeptides, as disclosed herein, is administered first, followed, after a period of hours (e.g., 1-12 hours), by administration of a unit dose of one or more additional therapeutic agents. In yet other embodiments, a unit dose of one or more additional therapeutic agents is administered first, followed, after a period of hours (e.g., 1-12 hours), by administration of a unit dose of one or more fusion polypeptides, or polynucleotides encoding or vectors expressing such fusion polypeptides, as disclosed herein.

In certain embodiments, one or more fusion polypeptides, or polynucleotides encoding or vectors expressing such fusion polypeptides, as disclosed herein, is combined with one or more additional therapeutic agents in a unitary dosage form for simultaneous or concurrent administration to a patient, for example as an aqueous formulation for intravenous, intramuscular, intradermal or subcutaneous administration. In certain embodiments, one or more fusion polypeptides, or polynucleotides encoding or vectors expressing such fusion polypeptides, as disclosed herein, is combined with one or more additional therapeutic agents in a unitary dosage form for simultaneous or concurrent administration to a patient, for example as an intrarectal suppository.

In certain embodiments, the one or more fusion polypeptides, or polynucleotides encoding or vectors expressing such fusion polypeptides, as disclosed herein, can be co-formulated or co-administered with one or more other compounds useful for treating HIV. In certain embodiments, the co-formulation or co-administration can comprise another active agent for treating HIV, such as an anti-HIV antibody, a toll-like receptor (TLR) agonist, an immune checkpoint inhibitor, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In certain embodiments, the one or more active agents are suitable for once daily dosing, weekly dosing, monthly dosing, every 3 months dosing, every four months dosing, bi-annual dosing, or annual dosing, as appropriate.

In some embodiments, the one or more fusion polypeptides, or polynucleotides encoding or vectors expressing such fusion polypeptides, as disclosed herein, and the one or more additional therapeutic agents may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, HIV Tat or Rev inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, HIV-1 Nef modulators, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

### Combination Drugs

In certain embodiments, the one or more fusion polypeptides, or polynucleotides encoding or vectors expressing such fusion polypeptides, as disclosed herein, are combined or co-administered with an HIV combination drug. Examples of combination drugs that can be employed with an agent of this disclosure include ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY (bictegravir+emtricitabine+tenofovir alafenamide), DOVATO, TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine and lamivudine; cabotegravir+rilpivirine; elpida (elsulfavirine; VM-1500; VM-1500A).

Examples of other drugs for treating HIV that can be combined with the one or more fusion polypeptides, or polynucleotides encoding or vectors expressing such fusion polypeptides, as disclosed herein, include acemannan, alisporivir, BanLec, deferiprone, Gamimune, metenkefalin, naltrexone, Prolastin, REP 9, RPI-MN, VSSP, H1viral, SB-728-T, 1,5-dicaffeoylquinic acid, rHIV7-shl-TAR-CCR5RZ, AAV-eCD4-Ig gene therapy, MazF gene therapy, BlockAide, ABX-464, AG-1105, APH-0812, BIT-225, CYT-107, HGTV-43, HPH-116, HS-10234, IMO-3100, IND-02, MK-1376, MK-2048, MK-4250, MK-8507, MK-8591, NOV-205, PA-1050040 (PA-040), PGN-007, SCY-635, SB-9200, SCB-719, TR-452, TEV-90110, TEV-90112, TEV-90111, TEV-90113, RN-18, Immuglo, and VIR-576.

### HIV Protease Inhibitors

In certain embodiments, the one or more fusion polypeptides, or polynucleotides encoding or vectors expressing such fusion polypeptides, as disclosed herein, are combined or co-administered with an HIV protease inhibitor. Examples of HIV protease inhibitors that can be combined with an agent of this disclosure include amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG-17, TMB-657 (PPL-100), T-169, BL-008, MK-8122, TMB-607, and TMC-310911.

### HIV Reverse Transcriptase Inhibitors

In certain embodiments, the one or more fusion polypeptides, or polynucleotides encoding or vectors expressing such fusion polypeptides, as disclosed herein, are combined or co-administered with a non-nucleoside or non-nucleotide inhibitor. Examples of HIV non-nucleoside or non-nucleotide inhibitors of reverse transcriptase that can be combined with an agent of this disclosure include dapivirine, delavirdine, delavirdine mesylate, doravirine, efavirenz, etravirine, lentinan, nevirapine, rilpivirine, ACC-007, AIC-292, KM-023, PC-1005, and elsulfavirine (VM-1500).

In certain embodiments, the one or more fusion polypeptides, or polynucleotides encoding or vectors expressing such fusion polypeptides, as disclosed herein, are combined or co-administered with an HIV nucleoside or nucleotide inhibitor. Examples of HIV nucleoside or nucleotide inhibitors of reverse transcriptase that can be combined with an agent of this disclosure include adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, MK-8591, MK-858, VM-2500 and KP-1461.

### HIV Integrase Inhibitors

In certain embodiments, the one or more fusion polypeptides, or polynucleotides encoding or vectors expressing such fusion polypeptides, as disclosed herein, are combined or co-administered with an HIV integrase inhibitor. Examples of HIV integrase inhibitors that can be combined with an agent of this disclosure include elvitegravir, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, dolutegravir, JTK-351, bictegravir, AVX-15567, cabotegravir (long-acting injectable), diketo quinolin-4-1 derivatives, integrase-LEDGF inhibitor, ledgins, M-522, M-532, NSC-310217, NSC-371056, NSC-48240, NSC-642710, NSC-699171, NSC-699172, NSC-699173, NSC-699174, stilbenedisulfonic acid, T 169, VM-3500 and cabotegravir.

In certain embodiments, the one or more fusion polypeptides, or polynucleotides encoding or vectors expressing such fusion polypeptides, as disclosed herein, are combined or co-administered with a HIV non-catalytic site, or allosteric, integrase inhibitor (NCINI). Examples of HIV non-catalytic site, or allosteric, integrase inhibitors (NCINI) that can be combined with an agent of this disclosure include CX-05045, CX-05168, and CX-14442.

### HIV Entry Inhibitors

In certain embodiments, the one or more fusion polypeptides, or polynucleotides encoding or vectors expressing such fusion polypeptides, as disclosed herein, are combined or co-administered with an HIV entry inhibitor. Examples of HIV entry (fusion) inhibitors that can be combined with an agent of this disclosure include cenicriviroc, CCR5 inhibitors, gp41 inhibitors, CD4 attachment inhibitors, gp120 inhibitors, and CXCR4 inhibitors.

In certain embodiments, the one or more fusion polypeptides, or polynucleotides encoding or vectors expressing such fusion polypeptides, as disclosed herein, are combined or co-administered with a CCR5 inhibitor. Examples of CCR5 inhibitors that can be combined with an agent of this disclosure include aplaviroc, vicriviroc, maraviroc, cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, and vMIP (Haimipu).

In certain embodiments, the one or more fusion polypeptides, or polynucleotides encoding or vectors expressing such fusion polypeptides, as disclosed herein, are combined or co-administered with a gp41 inhibitor. Examples of gp41 inhibitors that can be combined with an agent of this disclosure include albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer and sifuvirtide.

In certain embodiments, the one or more fusion polypeptides, or polynucleotides encoding or vectors expressing such fusion polypeptides, as disclosed herein, are combined or co-administered with a CD4 attachment inhibitor. Examples of CD4 attachment inhibitors that can be combined with an agent of this disclosure include ibalizumab and CADA analogs.

In certain embodiments, the one or more fusion polypeptides, or polynucleotides encoding or vectors expressing such fusion polypeptides, as disclosed herein, are combined or co-administered with a gp120 inhibitor. Examples of gp120 inhibitors that can be combined with an agent of this disclosure include Radha-108 (receptol) 3B3-PE38, BanLec, bentonite-based nanomedicine, fostemsavir tromethamine, IQP-0831, and BMS-663068.

In certain embodiments, the one or more fusion polypeptides, or polynucleotides encoding or vectors expressing such fusion polypeptides, as disclosed herein, are combined or co-administered with a CXCR4 inhibitor. Examples of CXCR4 inhibitors that can be combined with an agent of this disclosure include plerixafor, ALT-1188, N15 peptide, and vMIP (Haimipu).

In certain embodiments, the one or more fusion polypeptides, or polynucleotides encoding or vectors expressing such fusion polypeptides, as disclosed herein, are combined or co-administered with a HIV maturation inhibitor. Examples of HIV maturation inhibitors that can be combined with an agent of this disclosure include BMS-955176, GSK-3640254 and GSK-2838232.

### Latency Reversing Agents

In certain embodiments, the one or more fusion polypeptides, or polynucleotides encoding or vectors expressing such fusion polypeptides, as disclosed herein, are combined or co-administered with a latency reversing agent (LRA). Examples of latency reversing agents that can be combined with an agent of this disclosure include toll-like receptor (TLR) agonists (including TLR7 agonists, e.g., GS-9620), histone deacetylase (HDAC) inhibitors, proteasome inhibitors such as velcade, protein kinase C (PKC) activators, Smyd2 inhibitors, BET-bromodomain 4 (BRD4) inhibitors, ionomycin, IAP antagonists (inhibitor of apoptosis proteins, such as APG-1387, LBW-242), Second mitochondria-derived activator of caspases (SMAC; NCBI Gene ID: 56616) mimetics (including ciapavir, BI-891065, TL32711, LCL161, GDC-0917, HGS1029, AT-406), PMA, SAHA (suberanilohydroxamic acid, or suberoyl, anilide, and hydroxamic acid), NIZ-985, IL-15 modulating antibodies (including IL-15, IL-15 fusion proteins and IL-15 receptor agonists), JQ1, disulfiram, amphotericin B, and ubiquitin inhibitors such as largazole analogs, APH-0812, and GSK-343. Examples of PKC activators include indolactam, prostratin, ingenol B, and DAG-lactones.

### Histone Deacetylase (HDAC) Inhibitors

In certain embodiments, the one or more fusion polypeptides, or polynucleotides encoding or vectors expressing such fusion polypeptides, as disclosed herein, are combined or co-administered with an inhibitor of a histone deacetylase, e.g., histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CUDC-907 (fimepinostat), entinostat, givinostat, mocetinostat, panobinostat, pracinostat, quisinostat (JNJ-26481585), resminostat, ricolinostat, romidepsin, SHP-141, valproic acid (VAL-001), vorinostat, tinostamustine, remetinostat, entinostat.

### Capsid Inhibitor

In certain embodiments, the one or more fusion polypeptides, or polynucleotides encoding or vectors expressing such fusion polypeptides, as disclosed herein, are combined or co-administered with a capsid inhibitor. Examples of capsid inhibitors that can be combined with an agent of this disclosure include capsid polymerization inhibitors or capsid disrupting compounds, HIV nucleocapsid p7 (NCp7) inhibitors such as azodicarbonamide, HIV p24 capsid protein inhibitors, GS-6207 (lenacapavir), GS-CA1, AVI-621, AVI-101, AVI-201, AVI-301, and AVI-CAN1-15 series, and compounds described in this patent (GSK WO2019/087016).

### Immune Checkpoint Modulators

In certain embodiments, the one or more fusion polypeptides, or polynucleotides encoding or vectors expressing such fusion polypeptides, as disclosed herein, are combined or co-administered with one or more blockers, antagonists or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu, et al., J Exp Clin Cancer Res. (2018) 37:110). In various embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18(11):671-688).

Examples of immune checkpoint proteins or receptors include without limitation CD27 (NCBI Gene ID: 939), CD70 (NCBI Gene ID: 970), CD40 (NCBI Gene ID: 958), CD40LG (NCBI Gene ID: 959), CD47 (NCBI Gene ID: 961), CD48 (SLAMF2; NCBI Gene ID: 962), transmembrane and immunoglobulin domain containing 2 (TMIGD2, CD28H; NCBI Gene ID: 126259), CD84 (LY9B, SLAMF5; NCBI Gene ID: 8832), CD96 (NCBI Gene ID: 10225), CD160 (NCBI Gene ID: 11126), MS4A1 (CD20; NCBI Gene ID: 931), CD244 (SLAMF4; NCBI Gene ID: 51744); CD276 (B7H3; NCBI Gene ID: 80381); V-set domain containing T cell activation inhibitor 1 (VTCN1, B7H4; NCBI Gene ID: 79679); V-set immunoregulatory receptor (VSIR, B7H5, VISTA; NCBI Gene ID: 64115); immunoglobulin superfamily member 11 (IGSF11, VSIG3; NCBI Gene ID: 152404); natural killer cell cytotoxicity receptor 3 ligand 1 (NCR3LG1, B7H6; NCBI Gene ID: 374383); HERV-H LTR-associating 2 (HHLA2, B7H7; NCBI Gene ID: 11148); inducible T cell co-stimulator (ICOS, CD278; NCBI Gene ID: 29851); inducible T cell co-stimulator ligand (ICOSLG, B7H2; NCBI Gene ID: 23308); TNF receptor superfamily member 4 (TNFRSF4, OX40; NCBI Gene ID: 7293); TNF superfamily member 4 (TNFSF4, OX40L; NCBI Gene ID: 7292); TNFRSF8 (CD30; NCBI Gene ID: 943), TNFSF8 (CD30L; NCBI Gene ID: 944); TNFRSF10A (CD261, DR4, TRAILR1; NCBI Gene ID: 8797), TNFRSF9 (CD137; NCBI Gene ID: 3604), TNFSF9 (CD137L; NCBI Gene ID: 8744); TNFRSF10B (CD262, DR5, TRAILR2; NCBI Gene ID: 8795), TNFRSF10 (TRAIL; NCBI Gene ID: 8743); TNFRSF14 (HVEM, CD270; NCBI Gene ID: 8764), TNFSF14 (HVEML; NCBI Gene ID: 8740); CD272 (B and T lymphocyte associated (BTLA); NCBI Gene ID: 151888); TNFRSF17 (BCMA, CD269; NCBI Gene ID: 608), TNFSF13B (BAFF; NCBI Gene ID: 10673); TNFRSF18 (GITR; NCBI Gene ID: 8784), TNFSF18 (GITRL; NCBI Gene ID: 8995); MHC class I polypeptide-related sequence A (MICA; NCBI Gene ID: 100507436); MHC class I polypeptide-related sequence B (MICB; NCBI Gene ID: 4277); CD274 (CD274, PDL1, PD-L1; NCBI Gene ID: 29126); programmed cell death 1 (PDCD1, PD1, PD-1; CD279; NCBI Gene ID: 5133); cytotoxic T-lymphocyte associated protein 4 (CTLA4, CD152; NCBI Gene ID: 1493); CD80 (B7-1; NCBI Gene ID: 941), CD28 (NCBI Gene ID: 940); nectin cell adhesion molecule 2 (NECTIN2, CD112; NCBI Gene ID: 5819); CD226 (DNAM-1; NCBI Gene ID: 10666); Poliovirus receptor (PVR) cell adhesion molecule (PVR, CD155; NCBI Gene ID: 5817); PVR related immunoglobulin domain containing (PVRIG, CD112R; NCBI Gene ID: 79037); T cell immunoreceptor with Ig and ITIM domains (TIGIT; NCBI Gene ID: 201633); T cell immunoglobulin and mucin domain containing 4 (TIMD4; TIM4; NCBI Gene ID: 91937); hepatitis A virus cellular receptor 2 (HAVCR2, TIMD3, TIM3; NCBI Gene ID: 84868); galectin 9 (LGALS9; NCBI Gene ID: 3965); lymphocyte activating 3 (LAG3, CD223; NCBI Gene ID: 3902); signaling lymphocytic activation molecule family member 1 (SLAMF1, SLAM, CD150; NCBI Gene ID: 6504); lymphocyte antigen 9 (LY9, CD229, SLAMF3; NCBI Gene ID: 4063); SLAM family member 6 (SLAMF6, CD352; NCBI Gene ID: 114836); SLAM family member 7 (SLAMF7, CD319; NCBI Gene ID: 57823); UL16 binding protein 1 (ULBP1; NCBI Gene ID: 80329); UL16 binding protein 2 (ULBP2; NCBI Gene ID: 80328); UL16 binding protein 3 (ULBP3; NCBI Gene ID: 79465); retinoic acid early transcript 1E (RAET1E; ULBP4; NCBI Gene ID: 135250); retinoic acid early transcript 1G (RAET1G; ULBP5; NCBI Gene ID: 353091); retinoic acid early transcript 1L (RAET1L; ULBP6; NCBI Gene ID: 154064); killer cell lectin like receptor C1 (KLRC1, NKG2A, CD159A; NCBI Gene ID: 3821); killer cell lectin like receptor K1 (KLRK1, NKG2D, CD314; NCBI Gene ID: 22914); killer cell lectin like receptor C2 (KLRC2, CD159c, NKG2C; NCBI Gene ID: 3822); killer cell lectin like receptor C3 (KLRC3, NKG2E; NCBI Gene ID: 3823); killer cell lectin like receptor C4 (KLRC4, NKG2F; NCBI Gene ID: 8302); killer cell immunoglobulin like receptor, two Ig domains and long cytoplasmic tail 1 (KIR2DL1; NCBI Gene ID: 3802); killer cell immunoglobulin like receptor, two Ig domains and long cytoplasmic tail 2 (KIR2DL2; NCBI Gene ID: 3803); killer cell immunoglobulin like receptor, two Ig domains and long cytoplasmic tail 3 (KIR2DL3; NCBI Gene ID: 3804); killer cell immunoglobulin like receptor, three Ig domains and long cytoplasmic tail 1 (KIR3DL1, KIR, CD158E1; NCBI Gene ID: 3811) (e.g., Lirilumab (IPH2102/BMS-986015), IPH-4102); and killer cell lectin like receptor D1 (KLRD1; NCBI Gene ID: 3824).

In certain embodiments, the one or more fusion polypeptides, or polynucleotides encoding or vectors expressing such fusion polypeptides, as disclosed herein, are combined or co-administered with one or more blockers, antagonists or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). Lirilumab is an illustrative antibody that binds to and blocks KIR2DL1/2L3 receptors. In various embodiments, the fusion polypeptides, polynucleotides, vectors, LNPs, immunogenic compositions and/or pharmaceutical compositions, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu, et al., J Exp Clin Cancer Res. (2018) 37:110.

In certain embodiments, the one or more fusion polypeptides, or polynucleotides encoding or vectors expressing such fusion polypeptides, as disclosed herein, are combined or co-administered with one or more blockers, antagonists or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A), e.g., monalizumab (IPH2201); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis, et al., Semin Immunol. (2017) 31:64-75; Fang, et al., Semin Immunol. (2017) 31:37-54; and Chiossone, et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In certain embodiments, the one or more fusion polypeptides, or polynucleotides encoding or vectors expressing such fusion polypeptides, as disclosed herein, are combined or co-administered with anti-TIGIT antibodies, such as etigilimab, BMS-986207, tiragolumab (a.k.a., MTIG-7192A; RG-6058; RO 7092284), AGEN1307, AGEN1327, AGEN1777, COM-902, IBI-939, AB154, MG1131 and EOS884448 (EOS-448).

### TNF Receptor Superfamily (TNFRSF) Member Agonists or Activators

In certain embodiments, the one or more fusion polypeptides, or polynucleotides encoding or vectors expressing such fusion polypeptides, as disclosed herein, are combined or co-administered with one or more agonists of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Example anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Example anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Example anti-TNFRSF9 (4-1BB, CD137) antibodies that can be co-administered include without limitation urelumab, utomilumab (PF-05082566), AGEN2373 and ADG-106.

Example anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi- and Tri-Specific Natural Killer (NK)-Cell Engagers

In certain embodiments, the one or more fusion polypeptides, or polynucleotides encoding or vectors expressing such fusion polypeptides, as disclosed herein, are combined or co-administered with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BBAs appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices, et al., Methods Mol Biol. (2016) 1441:333-346; Fang, et al., Semin Immunol. (2017) 31:37-54. Examples of a trispecific NK cell engager (TRiKE) include OXS-3550, and CD16-IL-15-B7H3 TriKe.

### Indoleamine-Pyrrole-2,3-Dioxygenase (ID01) Inhibitors

In various embodiments, the one or more fusion polypeptides, or polynucleotides encoding or vectors expressing such fusion polypeptides, as disclosed herein, are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, and shIDO-ST, EOS-200271, KHK-2455, LY-3381916.

### Toll-Like Receptor (TLR) Agonists

In certain embodiments, the one or more fusion polypeptides, or polynucleotides encoding or vectors expressing such fusion polypeptides, as disclosed herein, are combined or co-administered with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-0509, GS-9620 (vesatolimod), LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Illustrative dual TLR7/TLR8 agonists that can be co-administered include CV8102, NKTR-262, telratolimod and BDB-001. Example TLR8 agonists that can be co-administered include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists that can be co-administered include without limitation AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. Examples of TLR4 agonist include G-100, and GSK-1795091. In some embodiments, the TLR agonist is a non-coding immunostimulatory polynucleotide selected from a pathogen-activated molecular pattern (PAMP), a cytosine-phosphate-guanosine (CpG) oligodeoxynucleotide, and an immunostimulatory RNA (isRNA, e.g., CV8102).

### STING Agonists, RIG-I and NOD2 Modulators

In certain embodiments, the one or more fusion polypeptides, or polynucleotides encoding or vectors expressing such fusion polypeptides, as disclosed herein, are combined or co-administered with a stimulator of interferon genes (STING) receptor (a.k.a, stimulator of interferon response cGAMP interactor 1 (STING1); transmembrane protein 173 (TMEM173); NCBI Gene ID: 340061) agonist. In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, 5,6-dimethylxanthenone-4-acetic acid (DMXAA), cyclic-GAMP (cGAMP) and cyclic-di-AMP.

In some embodiments, the additional therapeutic agent is an agonist of DExD/H-box helicase 58 (DDX58; a.k.a., RIG-I, RIG1, RIGI, RLR-1, SGMRT2; NCBI Gene ID: 23586). Illustrative RIG-I agonists include inarigivir soproxil (SB-9200; GS-9992); SB-40, SB-44, CV8102, ORI-7246, ORI-9350, ORI-7537, ORI-9020, ORI-9198, ORI-7170, RGT-100 and KIN1148, described by Hemann, et al., J Immunol May 1, 2016, 196 (1 Supplement) 76.1. Additional RIG-I agonists are described, e.g., in Elion, et al., Cancer Res. (2018) 78(21):6183-6195; and Liu, et al., J Virol. (2016) 90(20):9406-19. RIG-I agonists are commercially available, e.g., from Invivogen (invivogen.com). In some embodiments, the agents described herein are combined with a nucleotide binding oligomerization domain containing 2 (NOD2; NCBI Gene ID: 64127) agonist, such as inarigivir soproxil (SB-9200; GS-9992) and IR-103.

### LAG-3 and TIM-3 Inhibitors

In certain embodiments, the one or more fusion polypeptides, or polynucleotides encoding or vectors expressing such fusion polypeptides, as disclosed herein, are combined or co-administered with an anti-TIM-3 (hepatitis A virus cellular receptor 2; HAVCR2; CD366, HAVcr-2, KIM-3, SPTCL, TIM3, TIMID-3, TIMD3, Tim-3; NCBI Gene ID: 84868) antibody, such as TSR-022, LY-3321367, MBG-453, INCAGN-2390.

In certain embodiments, the one or more fusion polypeptides, or polynucleotides encoding or vectors expressing such fusion polypeptides, as disclosed herein, are combined or co-administered with anti-LAG-3 (lymphocyte-activating 3; LAG3; CD223; NCBI Gene ID: 3902) antibody, such as relatlimab (ONO-4482), LAG-525, MK-4280, REGN-3767, INCAGN2385.

### Interleukin or Cytokine Receptor Agonists

In certain embodiments, the one or more fusion polypeptides, or polynucleotides encoding or vectors expressing such fusion polypeptides, as disclosed herein, are combined or co-administered with a cytokine (e.g., interleukin) receptor agonist, such as IL-2, IL-7, IL-15, IL-10, IL-12, IL-18, IL-21, IFN-α, IFN-γ, GM-CSF, fins related receptor tyrosine kinase 3 (FLT3) receptor agonists, and combinations thereof. Examples of IL-2 receptor agonists that can be co-administered include proleukin (aldesleukin, IL-2); pegylated IL-2 (e.g., NKTR-214); modified variants of IL-2 (e.g., THOR-707), bempegaldesleukin, AIC-284, ALKS-4230, CUI-101, Neo-2/15. Examples of IL-15 receptor agonists that can be co-administered include ALT-803 (nogapendekin alfa), NKTR-255, and hetIL-15, interleukin-15/Fc fusion protein, AM-0015, NIZ-985, SO-C101, IL-15 Synthorin (pegylated Il-15), P-22339, and an IL-15-PD-1 fusion protein N-809. Examples of IL-7 receptor agonist that can be co-administered include CYT-107.

Examples of additional immune-based therapies that can be combined with an agent of this disclosure include interferon alfa; interferon alfa-2b; interferon alfa-n3; pegylated interferon alfa; interferon gamma; fins related tyrosine kinase 3 (FLT3) agonists (e.g., GS-3583, CDX-301); gepon; normferon, peginterferon alfa-2a, peginterferon alfa-2b, RPI-MN.

### Phosphatidylinositol 3-Kinase (PI3K) Inhibitors

In some embodiments, the immunogenic polypeptides, polynucleotides encoding such polypeptides, vectors, LNPs and immunogenic compositions comprising such polypeptides or polynucleotides, as described herein, are combined or co-administered with an inhibitor of a phosphatidylinositol-4,5-bisphosphate 3-kinase catalytic subunit, e.g., phosphatidylinositol-4,5-bisphosphate 3-kinase catalytic subunit alpha (PIK3CA, CLAPO, CLOVE, CWS5, MCAP, MCM, MCMTC, PBK, PI3K-alpha, p110-alpha; NCBI Gene ID: 5290); phosphatidylinositol-4,5-bisphosphate 3-kinase catalytic subunit beta (PIK3CB, P110BETA, PI3K, PBKBETA, PIK3C1; NCBI Gene ID: 5291); phosphatidylinositol-4,5-bisphosphate 3-kinase catalytic subunit gamma (PIK3CG, PI3CG, PI3K, PI3Kgamma, PIK3, p110gamma, p120-PBK; Gene ID: 5494); and/or phosphatidylinositol-4,5-bisphosphate 3-kinase catalytic subunit delta (PIK3CD, APDS, IMD14, P110DELTA, PI3K, p110D, NCBI Gene ID: 5293). In some embodiments, the PBK inhibitor is a pan-PI3K inhibitor. Examples of PBK inhibitors include without limitation, ACP-319, AEZA-129, AMG-319, AS252424, AZD8186, BAY 1082439, BEZ235, bimiralisib (PQR309), buparlisib (BKM120), BYL719 (alpelisib), carboxyamidotriazole orotate (CTO), CH5132799, CLR-457, CLR-1401, copanlisib (BAY 80-6946), DS-7423, duvelisib (IPI-145), fimepinostat (CUDC-907), gedatolisib (PF-05212384), GDC-0032, GDC-0084 (RG7666), GDC-0077, pictilisib (GDC-0941), GDC-0980, GSK2636771, GSK2269577, idelalisib (Zydelig®), INCB040093, INCB50465, IPI-443, IPI-549, KAR4141, LY294002, LY3023414, NERLYNX® (neratinib), nemiralisib (GSK2269557), omipalisib (GSK2126458, GSK458), OXY111A, panulisib (P7170, AK151761), PA799, perifosine (KRX-0401), Pilaralisib (SAR245408; XL147), puquitinib mesylate (XC-302), SAR260301, seletalisib (UCB-5857), serabelisib (INK-1117, MLN-1117, TAK-117), SF1126, sonolisib (PX-866), RG7604, rigosertib sodium (ON-01910 sodium), RP5090, tenalisib (RP6530), RV-1729, SRX3177, taselisib, TG100115, umbralisib (TGR-1202), TGX221, voxtalisib (SAR245409), VS-5584, WX-037, X-339, X-414, XL499, XL756, wortmannin, ZSTK474, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences).

### Alpha-4/Beta-7 Antagonists

In certain embodiments, the one or more fusion polypeptides, or polynucleotides encoding or vectors expressing such fusion polypeptides, as disclosed herein, are combined or co-administered with an alpha-4/beta-7 antagonist. Examples of Integrin alpha-4/beta-7 antagonists that can be combined with an agent of this disclosure include PTG-100, TRK-170, abrilumab, etrolizumab, carotegrast methyl, and vedolizumab.

### Inhibitor of CD47

In various embodiments, the one or more fusion polypeptides, or polynucleotides encoding or vectors expressing such fusion polypeptides, as disclosed herein, are combined or co-administered with an inhibitor of CD47 (IAP, MER6, OA3; NCBI Gene ID: 961; UniProt Q08722) or an agent that disrupts the binding of CD47 to SIRPα. Examples of CD47 inhibitors include without limitation to anti-CD47 mAbs (Vx-1004), anti-human CD47 mAbs (CNTO-7108), CC-90002, CC-90002-ST-001, humanized anti-CD47 antibody (Hu5F9-G4; magrolimab), NI-1701, NI-1801, RCT-1938, ALX-148, TTI-621, RRx-001, DSP-107, VT-1021, TTI-621, TTI-622, IMM-02 and SGN-CD47M, as well as CD47 targeting agents described in Intl. Patent Publ. Nos. WO199727873, WO199940940, WO2002092784, WO2005044857, WO2009046541, WO2010070047, WO2011143624, WO2012170250, WO2013109752, WO2013119714, WO2014087248, WO2015191861, WO2016022971, WO2016023040, WO2016024021, WO2016081423, WO2016109415, WO2016141328, WO2016188449, WO2017027422, WO2017049251, WO2017053423, WO2017121771, WO2017194634, WO2017196793, WO2017215585, WO2018075857, WO2018075960, WO2018089508, WO2018095428, WO2018137705, WO2018233575, WO2019027903, WO2019034895, WO2019042119, WO2019042285, WO2019042470, WO2019086573, WO2019108733, WO2019138367, WO2019144895, WO2019157843, WO2019179366, WO2019184912, WO2019185717, WO2019201236, WO2019238012, WO2019241732, WO2020019135, WO2020036977, WO2020043188 and WO2020009725.

Examples bi-specific antibodies targeting CD47 that can be combined or co-administered include without limitation IBI-322 (CD47/PD-L1), IMVM-0306 (CD47/CD20), TJ-L1C4 (CD47/PD-L1), HX-009 (CD47/PD-1), PMC-122 (CD47/PD-L1), PT-217, (CD47/DLL3), IMM-26011 (CD47/FLT3), IMM-0207 (CD47/VEGF), IMM-2902 (CD47/HER2), BH29xx (CD47/PD-L1), IMM-03 (CD47/CD20), IMM-2502 (CD47/PD-L1), HMBD-004B (CD47/BCMA), HMBD-004A (CD47/CD33). Examples of anti-CD47antibodies, such as IBI-188, TJC-4, SHR-1603, HLX-24, LQ-001, IMC-002, ZL-1201, IMM-01, B6H12, GenSci-059, TAY-018, PT-240, 1F8-GMCSF, SY-102, KD-015.

### HIV Targeting Antibodies

Examples of HIV antibodies, bispecific antibodies, and “antibody-like” therapeutic proteins that can be combined with an agent of this disclosure include DARTs®, DUOBODIES®, BITES®, XmAbs®, TandAbs®, Fab derivatives, bNAbs (broadly neutralizing HIV-1 antibodies), TMB-360, and those targeting HIV gp120 or gp41, antibody-Recruiting Molecules targeting HIV, anti-CD63 monoclonal antibodies, anti-GB virus C antibodies, anti-GP120/CD4, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), ibalizumab, Immuglo, MB-66.

In certain embodiments, the co-administered antibody or antigen-binding fragment thereof, or an antigen-binding molecule, is or is derived from human neutralizing antibodies (e.g., monoclonal) that target HIV-1. A “neutralizing antibody” is one that can neutralize the ability of HIV to initiate and/or perpetuate an infection in a host and/or in target cells in vitro. The disclosure provides neutralizing monoclonal human antibodies, wherein the antibody recognizes an antigen from HIV, e.g., a gp120 polypeptide. In certain embodiments, a “neutralizing antibody” may inhibit the entry of HIV-1 virus, e.g., SF162 and/or JR-CSF, with a neutralization index >1.5 or >2.0 (Kostrikis L G et al., J. Virol., 70(1): 445-458 (1996)).

In some embodiments, the co-administered antibody or antigen-binding fragment thereof, or an antigen-binding molecule, is or is derived from human broadly neutralizing antibodies (e.g., monoclonal) that target HIV-1. By “broadly neutralizing antibodies” are meant antibodies that neutralize more than one HIV-1 virus species (from diverse clades and different strains within a clade) in a neutralization assay. A broad neutralizing antibody may neutralize at least 2, 3, 4, 5, 6, 7, 8, 9 or more different strains of HIV-1, the strains belonging to the same or different clades. Illustrative broadly neutralizing antibodies (bNAbs) which can be co-administered as an additional therapeutic agent in a combination therapy are described, e.g., in U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Illustrative bNAbs that can be co-administered include without limitation 12A12, 12A21, NIH45-46, bANC131, 8ANC134, 1B2530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Sajadi, et al., Cell. (2018) 173(7):1783-1795; Sajadi, et al., J Infect Dis. (2016) 213(1):156-64; Klein et al., Nature, 492(7427): 118-22 (2012), Horwitz et al., Proc Natl Acad Sci USA, 110(41): 16538-43 (2013), Scheid, et al., Science, 333: 1633-1637 (2011), Scheid, et al., Nature, 458:636-640 (2009), Eroshkin et al, Nucleic Acids Res., 42 (Database issue):Dl 133-9 (2014), Mascola et al., Immunol Rev., 254(1):225-44 (2013), such as 2F5, 4E10, M66.6, CAP206-CH12, 10E81 (all of which bind the MPER of gp41); PG9, PG16, CH01-04 (all of which bind V1V2-glycan), 2G12 (which binds to outer domain glycan); b12, HJ16, CH103-106, VRC01-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site), which are hereby incorporated herein by reference in their entireties for all purposes.

In some embodiments, the one or more fusion polypeptides, or polynucleotides encoding or vectors expressing such fusion polypeptides, as disclosed herein, are combined or co-administered with a broadly neutralizing antibody (bNAb)) that binds to an epitope or region of gp120 selected from the group consisting of: (i) the third variable loop (V3) and/or high mannose patch comprising a N332 oligomannose glycan; (ii) second variable loop (V2) and/or Env trimer apex; (iii) CD4 binding site (CD4bs); (iv) gp120/gp41 interface; or (v) silent face of gp120. The foregoing epitopes or regions of gp120 bound by broadly neutralizing antibodies are described, e.g., in McCoy, Retrovirology (2018) 15:70; Sok and Burton, Nat Immunol. 2018 19(11):1179-1188; Possas, et al., Expert Opin Ther Pat. 2018 July; 28(7):551-560; and Stephenson and Barouch, Curr HIV/AIDS Rep (2016) 13:31-37, which are hereby incorporated herein by reference in their entirety for all purposes.

In some embodiments, the one or more fusion polypeptides, or polynucleotides encoding or vectors expressing such fusion polypeptides, as disclosed herein, are combined or co-administered with a broadly neutralizing antibody (bNAb) that binds to an epitope or region of gp120 in the third variable loop (V3) and/or high mannose patch comprising a N332 oligomannose glycan and competes with or comprises VH and VL regions from an antibody selected from the group consisting of GS-9722, PGT-121.60, PGT-121.66, PGT-121, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03. Additional broadly neutralizing antibodies that bind to gp120 in the third variable loop (V3) and/or high mannose patch comprising a N332 oligomannose glycan and which can be used as the second antibody or antigen-binding fragment thereof are described, e.g., in WO 2012/030904; WO 2014/063059; WO 2016/149698; WO 2017/106346; WO 2018/075564, WO 2018/125813 and WO 2018/237148, which are hereby incorporated herein by reference in their entireties for all purposes.

In some embodiments, the one or more fusion polypeptides, or polynucleotides encoding or vectors expressing such fusion polypeptides, as disclosed herein, are combined or co-administered with a broadly neutralizing antibody (bNAb)) that binds to an epitope or region of gp120 in the CD4 binding site (CD4bs) and competes with or comprises CDRs and/or VH and VL regions from an antibody selected from the group consisting of b12, F105, VRC01, VRC07, VRC07-523, VRC03, VRC06, VRC06b01 VRC08, VRC0801, NIH45-46, GS-9723, 3BNC117, 3BNC60, VRC-PG04, PGV04; CH103, 44-VRC13.01, 1NC9, 12A12, N6, N49-P7, NC-Cow1, IOMA, CH235 and CH235.12, N49P6, N49P7, N49P11, N49P9 and N60P25.

In some embodiments, the one or more fusion polypeptides, or polynucleotides encoding or vectors expressing such fusion polypeptides, as disclosed herein, are combined or co-administered with a broadly neutralizing antibody (bNAb)) that binds to an epitope or region of gp120 in the second variable loop (V2) and/or Env trimer apex and competes with or comprises VH and VL regions from an antibody selected from the group consisting of PG9, PG16, PGC14, PGG14, PGT-142, PGT-143, PGT-144, PGT-145, CH01, CH59, PGDM1400, CAP256, CAP256-VRC26.08, CAP256-VRC26.09, CAP256-VRC26.25, PCT64-24E and VRC38.01. Additional broadly neutralizing antibodies that bind to gp120 in the second variable loop (V2) and/or Env trimer apex and which can be used as the second antibody or antigen-binding fragment thereof are described, e.g., in WO 2010/107939; WO 2012/030904; WO 2018/075564 and WO 2018/125813, which are hereby incorporated herein by reference in their entireties for all purposes.

In some embodiments the one or more fusion polypeptides, or polynucleotides encoding or vectors expressing such fusion polypeptides, as disclosed herein, are combined or co-administered with a broadly neutralizing antibody (bNAb)) that binds to an epitope or region of gp120 in the gp120/gp41 interface and competes with or comprises VH and VL regions from an antibody selected from the group consisting of PGT-151, CAP248-2B, 35022, 8ANC195, ACS202, VRC34 and VRC34.01. Additional broadly neutralizing antibodies that bind to gp120 in the gp120/gp41 interface and which can be used as the second antibody or antigen-binding fragment thereof are described, e.g., in WO 2011/038290; WO 2012/030904 and WO2017/079479, which are hereby incorporated herein by reference in their entireties for all purposes.

In some embodiments, the one or more fusion polypeptides, or polynucleotides encoding or vectors expressing such fusion polypeptides, as disclosed herein, combined or co-administered with a broadly neutralizing antibody (bNAb)) that binds to an epitope or region of the gp120 silent face and competes with or comprises VH and VL regions from an antibody selected from the group consisting of VRC-PG05 and SF12. See, e.g., Schoofs, et al., “Broad and Potent Neutralizing Antibodies Recognize the Silent Face of the HIV Envelope,” Immunity (2019) May 14. pii: S1074-7613(19)30194-3 (PMID 31126879).

In some embodiments, the one or more fusion polypeptides, or polynucleotides encoding or vectors expressing such fusion polypeptides, as disclosed herein, are combined or co-administered with a broadly neutralizing antibody (bNAb)) that binds to an epitope or region of gp41 in the membrane proximal region (MPER). Additional broadly neutralizing antibodies that bind to gp41 in the MPER and which can be used as the second antibody or antigen-binding fragment thereof are described, e.g., in WO 2011/034582; WO 2011/038290; WO 2011/046623 and WO 2013/070776, which are hereby incorporated herein by reference in their entireties for all purposes.

In some embodiments, the one or more fusion polypeptides, or polynucleotides encoding or vectors expressing such fusion polypeptides, as disclosed herein, are combined or co-administered with a broadly neutralizing antibody (bNAb)) that binds to an epitope or region of gp41 in the membrane proximal region (MPER) and competes with or comprises VH and VL regions from an antibody selected from the group consisting of 10E8, 10E8v4, 10E8-5R-100cF, 4E10, DH511.11P, 2F5, 7b2, and LN01.

In some embodiments, the one or more fusion polypeptides, or polynucleotides encoding or vectors expressing such fusion polypeptides, as disclosed herein, are combined or co-administered with a broadly neutralizing antibody (bNAb)) that binds to an epitope or region of the gp41 fusion peptide and competes with or comprises VH and VL regions from an antibody selected from the group consisting of VRC34 and ACS202.

Examples of additional antibodies that can be co-administered include bavituximab, UB-421, BF520.1, CH01, CH59, C2F5, C4E10, C2F5+C2G12+C4E10, 3BNC117, 3BNC117-LS, 3BNC60, DH270.1, DH270.6, D1D2, 10-1074-LS, GS-9722, DH411-2, BG18, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM12, PGDM21, PCDN-33A, 2Dm2m, 4Dm2m, 6Dm2m, PGDM1400, MDX010 (ipilimumab), VRC01, VRC-01-LS, A32, 7B2, 10E8, VRC-07-523, VRC07-523LS, VRC24, VRC41.01, 10E8VLS, 3810109, 10E8v4, IMC-HIV, iMabm36, eCD4-Ig, IOMA, CAP256-VRC26.25, DRVIA7, VRC-HIVMAB080-00-AB, VRC-HIVMAB060-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Example of HIV bispecific and trispecific antibodies include MGD014, B12BiTe, TMB-bispecific, SAR-441236, VRC-01/PGDM-1400/10E8v4, 10E8.4/iMab, 10E8v4/PGT121-VRC01.

In some embodiments, the bNAbs can be expressed in vivo in the patient. Examples of in vivo delivered bNAbs include AAV8-VRC07; mRNA encoding anti-HIV antibody VRC01; and engineered B-cells encoding 3BNC117 (Hartweger et al, J. Exp. Med. 2019, 1301).

### Pharmacokinetic Enhancers

In certain embodiments, the one or more fusion polypeptides, or polynucleotides encoding or vectors expressing such fusion polypeptides, as disclosed herein, are combined with a pharmacokinetic enhancer. Examples of pharmacokinetic enhancers that can be combined with an agent of this disclosure include cobicistat and ritonavir.

### Additional Therapeutic Agents

Examples of additional therapeutic agents that can be combined with the one or more fusion polypeptides, or polynucleotides encoding or vectors expressing such fusion polypeptides, as disclosed herein, include the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

### HIV Vaccines

In certain embodiments, the one or more fusion polypeptides, or polynucleotides encoding or vectors expressing such fusion polypeptides, as disclosed herein, are combined with an HIV vaccine. Examples of HIV vaccines that can be combined with an agent of this disclosure include peptide vaccines, recombinant subunit protein vaccines, live vector vaccines, DNA vaccines, CD4-derived peptide vaccines, vaccine combinations, adenoviral vector vaccines (an adenoviral vector such as Ad5, Ad26 or Ad35), simian adenovirus (chimpanzee, gorilla, rhesus i.e. rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of HIV vaccines that can be co-administered include: rgp120 (AIDSVAX), ALVAC HIV (vCP1521)/AIDSVAX B/E (gp120) (RV144), monomeric gp120 HIV-1 subtype C vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), gp140[delta]V2.TV1+MF-59, rVSVIN HIV-1 gag vaccine, SeV-Gag vaccine, AT-20, DNK-4, ad35-Grin/ENV, TBC-M4, HIVAX, HIVAX-2, NYVAC-HIV-PT1, NYVAC-HIV-PT4, DNA-HIV-PT123, rAAV1-PG9DP, GOVX-B11, GOVX-B21, TVI-HIV-1, Ad-4 (Ad4-env Clade C+Ad4-mGag), Paxvax, EN41-UGR7C, EN41-FPA2, PreVaxTat, AE-H, MYM-V101, CombiHIVvac, ADVAX, MYM-V201, MVA-CMDR, DNA-Ad5 gag/pol/nef/nev (HVTN505), MVATG-17401, ETV-01, CDX-1401, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp160 HIV vaccine, RNActive HIV vaccine, SCB-703, Tat Oyi vaccine, TBC-M4, UBI HIV gp120, Vacc-4x+romidepsin, variant gp120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, arenavirus vector-based vaccines (e.g., described in WO 2009/083210; WO 2015/183895; WO 2016/075250; WO 2017/198726; and U.S. Pat. No. 9,943,585), MVA-BN HIV-1 vaccine regimen, UBI HIV gp120, mRNA based prophylactic vaccines, and TBL-1203HI.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In one embodiment, an agent disclosed herein, or a pharmaceutically acceptable salt thereof, is combined with one, two, three, four or more additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY (bictegravir+emtricitabine+tenofovir alafenamide), adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, the one or more fusion polypeptides, or polynucleotides encoding or vectors expressing such fusion polypeptides, as disclosed herein, are combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In a certain embodiment, the one or more fusion polypeptides, or polynucleotides encoding or vectors expressing such fusion polypeptides, as disclosed herein, are combined with abacavir sulfate, tenofovir, tenofovir disoproxil, tenofovir disoproxil fumarate, tenofovir disoproxil hemifumarate, tenofovir alafenamide, or tenofovir alafenamide hemifumarate.

In another embodiment, the one or more fusion polypeptides, or polynucleotides encoding or vectors expressing such fusion polypeptides, as disclosed herein, are combined with tenofovir, tenofovir disoproxil, tenofovir disoproxil fumarate, tenofovir alafenamide, or tenofovir alafenamide hemifumarate.

In yet another embodiment, the one or more fusion polypeptides, or polynucleotides encoding or vectors expressing such fusion polypeptides, as disclosed herein, are combined with a first additional therapeutic agent selected from the group consisting of abacavir sulfate, tenofovir, tenofovir disoproxil, tenofovir disoproxil fumarate, tenofovir alafenamide, and tenofovir alafenamide hemifumarate, and a second additional therapeutic agent selected from the group consisting of emtricitabine and lamivudine.

In another embodiment, the one or more fusion polypeptides, or polynucleotides encoding or vectors expressing such fusion polypeptides, as disclosed herein, are combined with a first additional therapeutic agent selected from the group consisting of tenofovir, tenofovir disoproxil, tenofovir disoproxil fumarate, tenofovir alafenamide, and tenofovir alafenamide hemifumarate, and a second additional therapeutic agent, wherein the second additional therapeutic agent is emtricitabine.

the one or more fusion polypeptides, or polynucleotides encoding or vectors expressing such fusion polypeptides, as disclosed herein, are combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the one or more fusion polypeptides, or polynucleotides encoding or vectors expressing such fusion polypeptides, as disclosed herein, are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of dendritic cell therapy include AGS-004. CCR5 gene editing agents include SB-728T. CCR5 gene inhibitors include Cal-1. In some embodiments, C34-CCR5/C34-CXCR4 expressing CD4-positive T-cells are co-administered with the one or more fusion polypeptides. In some embodiments, the agents described herein are co-administered with AGT-103-transduced autologous T-cell therapy or AAV-eCD4-Ig gene therapy.

### Gene Editors

In certain embodiments, the one or more fusion polypeptides, or polynucleotides encoding or vectors expressing such fusion polypeptides, as disclosed herein, are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T-Cell Therapy

In some embodiments, the agents described herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include VC-CAR-T, CMV-N6-CART, anti-CD4 CART-cell therapy, CD4 CAR+C34−CXCR4+CCR5 ZFN T-cells, autologous hematopoietic stem cells genetically engineered to express a CD4 CAR and the C46 peptide.

### TCR-T-Cell Therapy

In certain embodiments, the one or more fusion polypeptides, or polynucleotides encoding or vectors expressing such fusion polypeptides, as disclosed herein, are combined with a population of TCR-T-cells. TCR-T-cells are engineered to target HIV derived peptides present on the surface of virus-infected cells, for example ImmTAV.

### B-Cell Therapy

In certain embodiments, the one or more fusion polypeptides, or polynucleotides encoding or vectors expressing such fusion polypeptides, as disclosed herein, are combined with a population of B cells genetically modified to express broadly neutralizing antibodies, such as 3BNC117 (Hartweger et al, J. Exp. Med. 2019, 1301, Moffett et al., Sci. Immunol. 4, eaax0644 (2019) 17 May 2019).

### 8. Kits

Further provided are kits comprising one or more unitary doses of one or more of the fusion polypeptides, as described herein, or one or more polynucleotides encoding such fusion polypeptides, as described herein, or one or more vectors expressing such fusion polypeptides, as described herein. In some embodiments, the kit comprises two or more unitary doses of one or more of the fusion polypeptides, as described herein, or two or more polynucleotides encoding such fusion polypeptides, as described herein, or two or more vectors expressing such fusion polypeptides, as described herein. In some embodiments, the one or more unitary doses are in a single container. In some embodiments, the one or more unitary doses are in two or more separate containers. In certain embodiments, the unitary doses can be the same or different, e.g., can comprise the same or different unitary doses, e.g., can comprise polypeptides, polynucleotides, vectors or combinations thereof.

In various embodiments, the kit comprises one or more pharmaceutical packs or one or more containers (e.g., vials, ampules, pre-loaded syringes) containing one or more of the ingredients of the pharmaceutical compositions described herein, such as one or more of the fusion polypeptides, as described herein, or one or more polynucleotides encoding such fusion polypeptides, as described herein, or one or more vectors expressing such fusion polypeptides, as described herein. In various embodiments, the kit comprises one or more containers comprising the one or more of the fusion polypeptides, as described herein, or one or more polynucleotides encoding such fusion polypeptides, as described herein, or one or more vectors expressing such fusion polypeptides, as described herein, in an aqueous solution. In various embodiments, the kit comprises one or more containers comprising the one or more of the fusion polypeptides, as described herein, or one or more polynucleotides encoding such fusion polypeptides, as described herein, or one or more vectors expressing such fusion polypeptides, as described herein, in lyophilized form.

In some embodiments, the kit comprises one or more unitary doses of one or more viral vectors capable of expressing the fusion polypeptides. In some embodiments, the unitary doses of the one or more viral vectors are in the range of about 10³ to about 10¹² viral focus forming units (FFU) or plaque forming units (PFU) or infectious units (IU) or viral particles (vp), e.g. from about 10⁴ to about 10⁷ viral FFU or PFU or IU or vp, e.g. from about 10³ to about 10⁴, 10⁵, 10⁶, 10⁷, 10⁸, 10⁹, 10¹⁰, 10¹¹, 10¹², 10¹³, 10¹⁴ or 10¹⁵ viral FFU or PFU or IU or vp, per administration.

In some embodiments, the kit comprises two or more polynucleotides encoding or two or more viral vectors expressing the fusion polypeptides, the fusion polypeptides comprising: (1) One or more fusion polypeptides comprising or consisting of the following polypeptide segments in sequential order, from N-terminus to C-terminus, optionally joined or connected by one or more linkers: SEQ ID NOs: 70, 76, 94, 151 and 161; or SEQ ID NOs: 71, 77, 95, 152 and 162; and (2) One or more fusion polypeptides comprising or consisting of the following polypeptide segments in sequential order, from N-terminus to C-terminus, optionally joined or connected by one or more linkers: SEQ ID NOs: 188, 305, 28, 41, 294, 4, 176, 11, 319, 259, 282, 223, 213 and 37; SEQ ID NOs: 188, 305, 28, 41 and 294; SEQ ID NOs: 4, 176, 11, 319, 259, 282, 223, 213 and 37; SEQ ID NOs: 189, 306, 29, 42, 295, 5, 177, 12, 320, 260, 283, 224, 214 and 38; SEQ ID NOs: 189, 306, 29, 42 and 295; SEQ ID NOs: 5, 177, 12, 320, 260, 283, 224, 214 and 38; SEQ ID NOs: 305, 319, 259, 282, 223, 213, 294, 176 and 188; SEQ ID NOs: 306, 320, 260, 283, 224, 214, 295, 177 and 189; SEQ ID NOs: 305, 294, 223, 213, 176, 259, 319, 188 and 282; SEQ ID NOs: 306, 295, 224, 214, 177, 260, 320, 189 and 283; SEQ ID NOs: 305, 294, 319, 259, 282, 223, 176, and 188; SEQ ID NOs: 306, 295, 320, 260, 283, 224, 177 and 189; SEQ ID NOs: 305, 223, 294, 176, 259, 319, 188 and 282; or SEQ ID NOs: 306, 224, 295, 177, 260, 320, 189 and 283.

In some embodiments, the kit comprises two or more polynucleotides encoding or two or more viral vectors expressing the fusion polypeptides, the fusion polypeptides comprising: (1) One or more fusion polypeptides comprising an amino acid sequence of any one of SEQ ID NOs: 351-356 and 430, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 351-356 and 430; and (2) One or more fusion polypeptides comprising an amino acid sequence of any one of SEQ ID NOs: 357-366 and 407-410, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 357-366 and 407-410.

In some embodiments, the kit comprises one or more viral vectors, wherein each viral vector comprises two or more polynucleotides encoding two or more fusion proteins that are at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical, to the following amino acid sequences: SEQ ID NOs: 345 and 346; SEQ ID NOs: 347 and 348; SEQ ID NOs: 349 and 350; SEQ ID NOs: 351 and 352; SEQ ID NOs: 430 and 352; SEQ ID NOs: 357 and 358; SEQ ID NOs: 360 and 362; SEQ ID NOs: 359 and 361; SEQ ID NOs: 351 and 357; SEQ ID NOs: 351 and 358; SEQ ID NOs: 351 and 359; SEQ ID NOs: 351 and 360; SEQ ID NOs: 351 and 361; SEQ ID NOs: 351 and 362; SEQ ID NOs: 351 and 407; SEQ ID NOs: 351 and 408; SEQ ID NOs: 351 and 409; SEQ ID NOs: 351 and 410; SEQ ID NOs: 352 and 357; SEQ ID NOs: 352 and 358; SEQ ID NOs: 352 and 359; SEQ ID NOs: 352 and 360; SEQ ID NOs: 352 and 361; SEQ ID NOs: 352 and 362; SEQ ID NOs: 352 and 407; SEQ ID NOs: 352 and 408; SEQ ID NOs: 352 and 409; SEQ ID NOs: 352 and 410; SEQ ID NOs: 430 and 357; SEQ ID NOs: 430 and 358; SEQ ID NOs: 430 and 359; SEQ ID NOs: 430 and 360; SEQ ID NOs: 430 and 361; SEQ ID NOs: 430 and 362; SEQ ID NOs: 407 and 409; SEQ ID NOs: 407 and 408; SEQ ID NOs: 408 and 410; or SEQ ID NOs: 409 and 410.

In some embodiments, the kit comprises one or more polynucleotides encoding or one or more viral vectors expressing the fusion polypeptides, the fusion polypeptides comprising or consisting of the following polypeptide segments in sequential order, from N-terminus to C-terminus, optionally joined or connected by one or more linkers: SEQ ID NOs: 201, 78, 107, 96,229, 172, 327, 6, 333, 243, 331, 192, 265, 311, 137, 15, 123, 30, 336, 302, 153, 219, 298, 121, 230, 240, 60, 241, 276, 113, 99, 21, 217 and 215; SEQ ID NOs: 78, 296, 1, 339, 197, 329, 232, 323, 303, 234, 90, 261, 274, 238, 211, 325, 137, 227, 209, 190, 341, 57, 225, 27, 210, 119, 19, 165, 334, 117, 153, 10, 97 and 300; or SEQ ID NOs: 296, 1, 78, 197, 339, 227, 261, 274, 238, 325, 137, 329, 303, 234, 90, 232, 27, 57, 225, 323, 190, 341, 119, 19, 165, 334, 117, 153, 10, 97 and 300.

In some embodiments, the kit comprises one or more polynucleotides encoding or one or more viral vectors expressing the fusion polypeptides, the fusion polypeptides comprising or consisting of an amino acid sequence of any one of SEQ ID NOs: 367-377, 411, 422-424 and 431-435, or a sequence that is at least 80%, 81%, 82%, 83%, 84%, 85%, 86%, 87%, 88%, 89%, 90%, 91%, 92%, 93%, 94%, 95%, 96%, 97%, 98% or 99% identical to any one of SEQ ID NOs: 367-377, 411, 422-424 and 431-435.

In some embodiments, the kit further comprises one or more unitary doses of one or more additional therapeutic agents. For example, in some embodiments, the kit comprises one or more agonists or activators of one or more toll-like receptors (TLRs). In some embodiments, the TLR agonist or activator is selected from the group consisting of a TLR2 agonist, a TLR3 agonist, a TLR4 agonist, a TLR5 agonist, a TLR7 agonist, a TLR8 agonist and a TLR9 agonist. In some embodiments, the TLR7 agonist is selected from the group consisting of GS 9620 (vesatolimod), R848 (Resiquimod), DS-0509, LHC-165 and TMX-101 (imiquimod), and/or wherein the TLR8 agonist is selected from the group consisting of GS-9688, R848 (Resiquimod), CV8102 (dual TLR7/TLR8 agonist) and NKTR-262 (dual TLR7/TLR8 agonist). In some embodiments, the TLR9 agonist is selected from the group consisting of AST-008, cobitolimod, CMP-001, IMO-2055, IMO-2125, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. In some embodiments, the TLR agonist is a non-coding immunostimulatory polynucleotide selected from a pathogen-activated molecular pattern (PAMP), a cytosine-phosphate-guanosine (CpG) oligodeoxynucleotide, and an immunostimulatory RNA (isRNA, e.g., CV8102).

In some embodiments, the kit comprises one or more interleukin receptor agonists of an interleukin selected from IL-2, IL-7, IL-12, IL-15, IL-18, IL-21, IFN-α, IFN 7, colony stimulating factor 2 (CSF2; a.k.a., GM-CSF) and FLT3LG, e.g., one or more cytokines selected from the group consisting of IL-2, IL-7, IL-12, IL-15, IL-18, IL-21, IFN-α, IFN-γ, GM-CSF, FLT3LG, and combinations and functional variants thereof.

In some embodiments, the kit comprises one or more antagonists or inhibitors of an inhibitory immune checkpoint protein or receptor and/or one or more activators or agonists of a stimulatory immune checkpoint protein or receptor. In some embodiments, the one or more immune checkpoint proteins or receptors are selected from the group consisting of: CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGHT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAET1L; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); and SLAM family member 7 (SLAMF7). In some embodiments, the kit comprises one or more blockers, antagonists or inhibitors of one or more T-cell inhibitory immune checkpoint proteins or receptors. In some embodiments, the T-cell inhibitory immune checkpoint proteins or receptors are selected from the group consisting of CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). Lirilumab is an illustrative antibody that binds to and blocks KIR2DL1/2L3 receptors. In some embodiments, the kit comprises one or more agonists or activators of one or more T-cell stimulatory immune checkpoint proteins or receptors. In some embodiments, the T-cell stimulatory immune checkpoint proteins or receptors are selected from the group consisting of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the kit comprises one or more blockers, antagonists or inhibitors of one or more NK-cell inhibitory immune checkpoint proteins or receptors. In some embodiments, the NK-cell inhibitory immune checkpoint proteins or receptors are selected from the group consisting of killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A), e.g., monalizumab (IPH2201); and killer cell lectin like receptor D1 (KLRD1, CD94). In some embodiments, the kit comprises one or more agonists or activators of one or more NK-cell stimulatory immune checkpoint proteins or receptors. In some embodiments, the NK-cell stimulatory immune checkpoint proteins or receptors are selected from CD16, CD226 (DNAM-1); killer cell lectin like receptor K1 (KLRK1, NKG2D, CD314); and SLAM family member 7 (SLAMF7). In some embodiments, the one or more immune checkpoint inhibitors comprises a proteinaceous inhibitor of PD-L1 (CD274), PD-1 (PDCD1) or CTLA4. In some embodiments, the proteinaceous inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4) and AK-104 (CTLA4/PD-1). In some embodiments, the proteinaceous inhibitor of PD-L1 (CD274) or PD-1 (PDCD1) is selected from the group consisting of pembrolizumab, nivolumab, cemiplimab, pidilizumab, AB122 (zimberelimab), AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1). In some embodiments, the one or more immune checkpoint inhibitors comprises a small molecule inhibitor of CD274 (PDL1, PD-L1), programmed cell death 1 (PDCD1, PD1, PD-1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

In some embodiments, the kit comprises one or more anti-viral agents. In some embodiments, the one or more antiviral agents are selected from the group consisting of HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, and capsid inhibitors.

Optionally associated with such container(s) can be a notice in the form prescribed by a governmental agency regulating the manufacture, use or sale of pharmaceuticals or biological products, which notice reflects approval by the agency of manufacture, use or sale for human administration.

### 9. Methods of Designing Fusion Polypeptides Useful to Promote Antiviral Immune Responses

Provided are methods for designing a vaccine construct or an immunogen that is capable of eliciting an immune response in a human against one or more viral antigens. The immunogenic fusion polypeptides are designed employing a combination of computational, experiential and manual steps, and can be used to elicit an immune response against a highly variable virus. The design methods can be applied to creating an immunogen capable of inducing an immune response in a human against one or more viral antigens of a desired target virus, including without limitation human immunodeficiency virus (HIV), hepatitis B virus (HBV), human papillomavirus (HPV), herpes simplex virus (HSV), Ebola virus, Zika virus and Chikungunya virus. In different implementations, the methods provide a vaccine construct designed for (1) maximum epitope coverage of a broad-based population, referred to herein as a “population” construct or antigen; (2) maximum epitope coverage for a group of individuals sharing a defined set of HLA alleles, referred to herein as an “HLA-restricted” construct or antigen; or (3) maximum epitope coverage for an infected individual's virus by accounting for intra-patient variability in the virus, including the individual's unique complement of viral ‘quasi species’, referred to herein as a “personalized” construct or antigen. Preferably, the segments comprising each of the constructs represent one or more MHC class I and/or MHC class II T cell epitopes. Accordingly, the segments may be referred to herein as population, HLA-restricted, or personalized epitopes that can be combined and assembled into immunogenic fusion polypeptides.

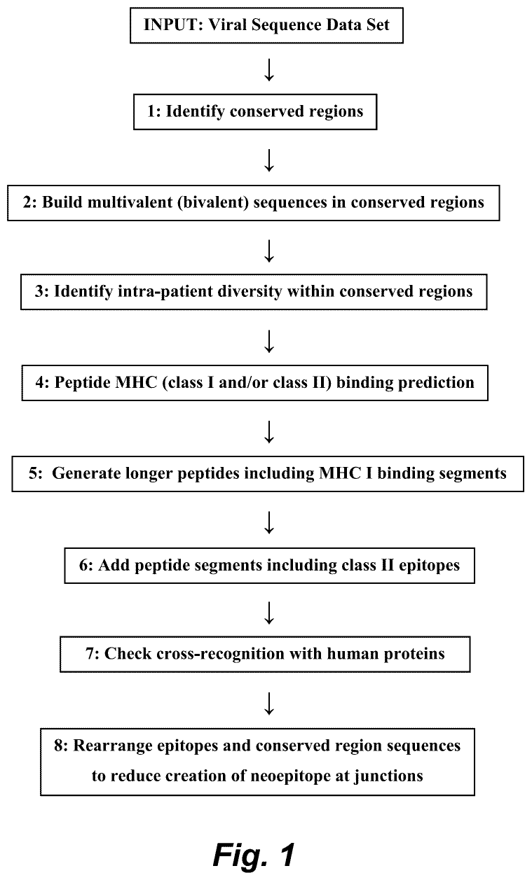

Most of the steps can be performed in silico, but some steps can be performed manually (e.g., inclusion and/or exclusion selections of certain polypeptide sequences; selection of linker or linkers) and may incorporate information based on experimental data (e.g., experimentally determined MHC class II epitopes). The input information is a viral sequence data set (e.g., for HIV, internal and publicly available HIV population data sets). As summarized in the flow chart of FIG. 1 and Table M below, the vaccine design methods involve at least 2, e.g., at least 3, 4, 5, 6, 7 or 8 of the steps of: 1. Identify conserved regions. All 9 amino acid segments (9-mers) are considered in naturally occurring viral sequences as potential T-cell epitopes. 9-mer positions having a conservation of at least 80% across interpatient viral populations, are identified as conserved regions and included for further analysis. 2. Build bivalent sequences from conserved regions. This can be done by employing a graph-based algorithm. 9-mers are assembled from conserved regions to include the maximum number of naturally occurring 9-mers. 3. Identify intra-patient diversity within conserved regions. This can be done using deep sequencing data. 4. Predict binding of identified polypeptide segments to human MHC alleles. 5. Generate longer peptide segments (e.g. 15 to 26 amino acids in length) including the polypeptide segments predicted to bind to human MHC class I molecules. 6. Include polypeptide segments predicted or shown to bind to MHC class II. 7. Evaluate and eliminate polypeptide segments having high sequence identity with (e.g., cross-recognition) host (e.g., human, dog, cat, horse) proteins. 8. Arrange polypeptide segments to reduce or avoid the creation of human-recognizable neoepitopes at junctions. This can be done by evaluating 9-mers around junctions for MHC class I binding and cross-recognition with host (e.g., human, dog, cat, horse) proteins.

TABLE M
Different Immunogen Design Approaches

| | | No deep sequencing data analysis | | | | Deep sequencing | |
|---|---|---|---|---|---|---|---|
| | | | | Example 3 | | data analysis | |
| | | | | single/multiple allele | | Example 5 | |
| | | | | | Long | Single/ | |
| | | | | Short | peptide | multiple | |
| | | Example 1 | Example 2 | peptide | (15- | allele | |
| | | Population- | Population- | (9-mers) | 30-mers) | long | Example 4 |
| Step | Step Description | based | based | approach | approach | peptide | Individual |
| 1 | Identify conserved regions | Y* | Y | Y | Y | Y | Y |
| 2 | Build bivalent sequences | Y | Y | Y | Y | Y | Y |
| 3 | Intra-patient diversity | N | N | N | N | Y | Y |
| 4 | MHC class I binding prediction | N | N | Y | Y | Y | Y |
| 5 | Long peptides for improved | N | N | N | Y | Y | Y |
| | presentation | | | | | | |
| 6 | Add class II epitopes | N | N | Y | Y | Y | Y |
| 7 | Cross-recognition analysis | N | N | Y | Y | Y | Y |
| 8 | Junctional response analysis | N | Y | Y | Y | Y | Y |

*Y: yes, included in method variation;
N: no not included in method variation

In addition to improving epitope coverage, the methods described herein further provide for selecting the most immunogenic epitopes from among the population, HLA-restricted, or personalized epitopes that are identified for incorporation into a final vaccine construct. In one aspect, these methods comprise screening a set of candidate polypeptide segments of a population, HLA-restricted, or personalized construct for MHC peptide binding affinity. MHC binding affinity can be predicted using one or more algorithms. Exemplary predictive algorithms include NetMHC (Vita et al. Nucleic Acids Res 2015 43:D405-D412), NetMHCpan (Andreatta and Nielsen Bioinformatics 2016 32:511-517), and MHCflurry (O'Donnell et al. Cell Syst 2018 7:12-132). Other T-cell epitope prediction tools are publicly available and are described, for example in Sanchez-Trincado et al. J. Immunology Res. 2017 Article ID 2680160. Additional methods for identifying MHC binding peptides include those employing machine learning tools, for example, as described in U.S. Pat. No. 10,055,540, WO 2019/104203 and the “EDGE” tool described in Bulik-Sullivan et al. Nature Biotechnology 2019 37:55.

In some implementations, the disclosure provides methods for producing a bivalent population or HLA-restricted construct designed both to capture the unique diversity of a viral proteome (e.g., HIV proteome) by providing mathematically determined and improved coverage of all potential T cell epitopes and to ensure that the epitopes in each polypeptide of the pair of constructed polypeptides retain the positional information of the original input viral sequences, e.g., by retaining the same order of the polypeptide segments as found in the naturally occurring viral proteome. The epitopes of the resulting pair of polypeptides will therefore more closely resemble those of the naturally occurring viral sequences, increasing the likelihood of their eliciting a relevant T cell response.

In some implementations, the disclosure provides methods for producing a bivalent HLA-restricted construct designed to capture the host genetic diversity driving antigen processing and T cell recognition by modeling epitope generation across a range of host HLA alleles.

Generally, the methods described here comprise initially providing a set of mathematically determined and improved potential T cell epitopes (“PTE”) in terms of their coverage of all PTEs in a population of viral proteome sequences, using a graph-based approach. Unlike similar graph-based approaches to vaccine design, the approach described here builds segments of connected PTE's using only adjacent PTE's that are also adjacent in the natural sequences. This provides constructs that retain the positional information of the PTE's within the source set of sequences. Also unlike other graph-based approaches, the methods described here simultaneously build a bivalent construct to provide maximal coverage of the most highly conserved PTEs in the population. The result is an initial bivalent vaccine construct that advantageously maximizes highly conserved PTEs that are most likely to be highly similar to conserved epitopes in the natural sequences. Further advantageously, the use of only the most highly conserved PTEs reduces the likelihood of escape mutants because the highly conserved sequences are more likely to be essential for viral function.

The initial bivalent construct produced by these methods may itself be used as a vaccine, or it may serve as the basis for a further construct, such as an HLA-restricted construct or a personalized construct, as described in more detail below.

The methods described herein generally begin with the identification of a conserved region bivalent sequences, using a process referred to herein as the “Conservation Analysis” or “Conservation Algorithm”. The methods further generally comprise a step of building a bivalent vaccine construct having maximal epitope coverage while retaining the positional information of the PTE's from the natural sequences, using a process referred to referred to herein as a “Conserved Walking Algorithm” or “CWA”. Thus, in some implementations, an initial step in the method is identifying a set of all conserved regions in a viral proteome for a selected set of viral genes. In implementations for designing a fusion polypeptide to elicit an immune response against HIV-1, the set of HIV-1 viral genes is selected from two or more of Gag, Pol, Env, and Nef. In some implementations, the set of viral genes consists of Gag, Pol, Env, and Nef. In some implementations, the set of viral genes consists of Gag, Pol and Nef. In some implementations, the set of viral genes consists of Gag and Nef or Pol and Env, or solely Pol.

In accordance with the methods described here, a population of viral proteome sequences is first aligned to a reference sequence, for example, the HIV reference sequence HXB2 identified by GenBank No. Accession K03455. Reference sequences for polypeptides encoded by the Env, Gag, Nef and Pol genes are provided herein as SEQ ID NOs: 403-406, respectively. The initial input or ‘source’ population of viral proteome sequences consists of sequences obtained from naturally occurring viruses. Such sequences are publicly available, for example, from the HIV Databases maintained by the Los Alamos National Laboratory, the U.S. Dept. of Health and Human Services, and the National Institutes of Health. In some implementations of the methods described herein, the source viral sequences may consist of sequences corresponding to a specific viral group and/or clade. In some implementations, in order to improve the identification of conserved regions and sequences, the input viral sequences may be restricted to a single viral clade and/or group. In some implementations, the input viral sequences are restricted to Group M clade B sequences.

The alignment of the source viral sequences to the reference sequence may be accomplished using a multiple alignment algorithm, for example, the fast Fourier transform algorithm, MAFFT. Katoh et al. 2002 Nucleic Acids Res. 30 (14):3059-66. The base MAFFT software is publicly available and distributed, e.g., under the Berkeley Software Distribution (BSD) license.

Next, the Conservation Algorithm is applied to the aligned sequences. For each sequence in the alignment, starting from the first amino acid position, the method shifts one amino acid position at a time and creates all possible amino acid segments that are 9 amino acids in length, referred to herein as “9-mers”. The algorithm thus creates, for each sequence in the alignment, a set of 9-amino acid subsequences (“9-mers”) starting with the N-terminal amino acid, each subsequence overlapping the preceding subsequence by eight amino acids such that each sequence of length l in the alignment contains (l-8) 9-mers.

Next, for each 9-mer position, the method identifies the two most common unique 9-mers and their prevalence in the aligned set of source viral proteome sequences. Stated another way, starting at position i the two most common unique 9-mers at each position are identified based on their frequency, calculated as the number of times the unique 9-mer occurs at position i in the alignment divided by the total number of sequences in the alignment.

Computationally, each sequence of length l, contains l-8 9-mers. We define all the 9-mers starting at position i as sᵢⱼ and frequency as fᵢⱼ, j=1, 2, 3, . . . m. In total there are m unique 9-mers at position i. Each two unique 9-mers (sᵢᵤ, sᵢᵥ) can constitute a 9-mer pair and its frequency is fᵢᵤ+fᵢᵥ. And each 9-mer itself can constitute a 9-mer pair as (sᵢᵤ, sᵢᵤ) and its frequency is fᵢᵤ. Thus, in total, there are m+(m−1)+(m−2)+ . . . +2+1=m*(m+1)/2 9-mer pairs at each position.

The method then calculates the bivalent conservation for each 9-mer position by summing up the proportions of aligned set of source viral proteome sequences containing either of the two most common 9-mers. To do this, a “bivalent conservation” is calculated for each position by summing the proportion of sequences in the alignment containing either of the two most common unique 9-mers. As used herein, “bivalent conservation” refers to the percentage of sequences containing exactly the same 9 amino acid segments (9-mers) as either of the two most prevalent ones in a 9-mer position.

Next, a new alignment of conserved regions is created by extracting the sequences in the alignment having a desired bivalent conservation, for example, a bivalent conservation of greater than 80% or greater than 90%, meaning that the two most common 9-mers at position i account for more than 80% or more than 90% of the 9-mers at that position in the new alignment of conserved regions. Stated another way, the method identifies the conserved regions in the new alignment as those in which the sum of the frequencies of the two most common 9-mers at each position is greater than a certain cutoff, e.g., greater than 80% or greater than 90%. Thus, the method also calculates the frequency of each pair of unique 9-mers at each position in the new alignment of conserved regions.

In some implementations, further selection criteria may be applied to the conserved regions, such as restricting to regions having greater than 90% conservation and removing short segments of less than 35 amino acids.

Using this modified set of conserved regions, certain implementations of the method apply the CWA to build bivalent sequence constructs. The CWA connects 9-mer pairs in adjacent positions of the alignment of conserved regions that share an overlap of eight amino acids.

Computationally, each 9-mer s contains 9 amino acids, we write s[x:y] to represent the amino acid subsequence from position x to y, y−x+1 amino acids in total:

sᵢᵤ[2:9]=sᵢ₊₁ₚ[1:8] and sᵢᵥ[2:9]=s_{i+1q}[1:8]

or

sᵢᵤ[2:9]=s_{i+1q}[1:8] and sᵢᵥ[2:9]=sᵢ₊₁ₚ[1:8].

Next, the algorithm builds a directed acyclic graph in which each 9-mer pair is a node and the edges between adjacent nodes are formed from the connected 9-mer pairs in the adjacent positions with the weight of each edge equal to the frequency of the downstream 9-mer pair. This directed acyclic graph is a positional De Brujin graph. Such graphs have been described in connection with assemblies of next generation sequencing data, for example as described in Ronen et al., Bioinformatics 2012 28:188-196. The method further adds a source node, connecting it with all of the nodes in the first position; and a sink node, connecting it with all of the nodes in the last position. The weights are then negated and the optimal path is found from the source node to the sink node, where the optimal path is defined as the path that has the largest sum of the frequencies of all 9-mer pairs among all the paths from the source node to the sink node. The task of finding the optimal path is performed, for example, using the Bellman-Ford algorithm. Generally, the Bellman-Ford algorithm computes the shortest paths from a single source vertex to all of the other vertices in a weighted directed graph which is made up of a set of vertices connected by edges, where the edges have a direction associated with them. The computational steps can be summarized as follows:
(4-1) Treat each 9-mer pair as a node, and build edges between adjacent nodes in Step 3;
(4-2) Adding a source node and connect it with all the nodes at the 1st position;
(4-3) Adding a sink node and connect it with all the nodes at the last position;
(4-4) Weight of each edge equals to the frequency of downstream 9-mer pair;
(4-5) Negating all the weights and finding the optimal path using the Bellman-Ford algorithm.

A further step of the method is to build bivalent vaccine sequences based on the optimal bivalent 9-mer pair path and connect two 9-mers in adjacent positions within the optimal bivalent 9-mer pair path if they share an overlap of 8 amino acids. A bivalent construct is built by connecting two 9-mers in adjacent positions within the optimal bivalent 9-mer path if they share an overlap of eight amino acids, thereby creating two sequences of connected 9-mers which together form the bivalent construct. The connected adjacent 9-mer pairs all have an 8 amino acid overlap, so they will be assembled into two sequences. For example, one 9-mer pair (AIIIIIIIS (SEQ ID NO: 464), MIIIIIIII (SEQ ID NO: 465)) can be connected with another 9-mer pair (IIIIIIISK (SEQ ID NO: 466), IIIIIIIIR (SEQ ID NO: 467) ) and make two sequences (bivalent sequences): AIIIIIIISK (SEQ ID NO: 468) and MIIIIIIIIR (SEQ ID NO: 469).

Computationally, the methodology can be described as a positional De Brujin graph based bivalent vaccine sequence design algorithm comprising the following 5 basic steps:
Step 1: align all the population sequences.
Step 2: for each 9-mer position, pull out all the unique 9-mers and their frequencies, and build 9-mer pair sets with frequencies. Each sequence of length l, contains l-8 9-mers. We define all the 9-mers starting at position i as sᵢⱼ and frequency as fᵢⱼ, j=1,2,3, . . . m. In total there are m unique 9-mers at position i. Each two unique 9-mers (sᵢᵤ, sᵢᵥ) can constitute a 9-mer pair and its frequency is fᵢᵤ+fᵢᵥ. And each 9-mer itself can constitute a 9-mer pair as (sᵢᵤ, sᵢᵤ) and its frequency is fᵢᵤ. Thus, in total, there are m+(m−1)+(m−2)+ . . . +2+1=m*(m+1)/2 9-mer pairs at each position.
Step 3: connect 9-mer pairs in adjacent positions if they do not have any conflicting amino acids. As used herein, “conflicting amino acid residues” refers to different amino acid residues at overlapped positions between two 9-mers. Each 9-mer s contains 9 amino acids, we write s[x:y] to represent the amino acid subsequence from position x to y, y−x+1 amino acids in total:

sᵢᵤ[2:9]=sᵢ₊₁ₚ[1:8] and sᵢᵥ[2:9]=s_{i+1q}[1:8]

or

sᵢᵤ[2:9]==s_{i+1q}[1:8] and sᵢᵥ[2:9]=sᵢ₊₁ₚ[1:8]
Step 4: find the optimal path from the 1st 9-mer position to the last position in terms of the sum of the frequencies of all the 9-mers within the path. The basic idea is to model the maximum coverage bivalent vaccine construction problem as a classic graph theory problem where the solution is finding the minimum path in a directed acyclic graph.
Step 5: build bivalent vaccine sequences based on the optimal bivalent 9-mer pair path and connect two 9-mers in adjacent positions within the optimal bivalent 9-mer pair path if they share an overlap of 8 amino acids. Take for example the following cases:
Case 1: if sᵢᵤ[2:9]=sᵢ₊₁ₚ[1:8] and sᵢᵥ[2:9]=s_{i+1q}[1:8], connect sᵢᵤ with sᵢ₊₁ₚ and sᵢᵥ with s_{i+1q};
Case 2: if sᵢᵤ[2:9]=s_{i+1q}[1:8] and sᵢᵥ[2:9]=sᵢ₊₁ₚ[1:8], connect sᵢᵤ with s_{i+1q} and sᵢᵥ with s_{i+1q};
Case 3: if sᵢᵤ[2:9]=sᵢ₊₁ₚ[1:8] and sᵢᵥ[2:9]=s_{i+1q}[1:8] and sᵢᵤ[2:9]=s_{i+1q}[1:8] and sᵢᵥ[2:9]=sᵢ₊₁ₚ[1:8], the selection of connection is based on the prevalence of the two connections in natural sequences:

Denote the prevalence of the co-existence of sᵢₓ and s_{i+1y} in input sequences as C_{ixy};
If Cᵢᵤₚ+C_{ivq}>C_{iuq}+Cᵢᵥₚ, connect sᵢᵤ with sᵢ₊₁ₚ and sᵢᵥ with s_{i+1q};
If C_{iuq}+Cᵢᵥₚ>Cᵢᵤₚ+C_{ivq}, connect sᵢᵤ with s_{i+1q} and sᵢᵥ with sᵢ₊₁ₚ;
If Cᵢᵤₚ+C_{ivq}=C_{ivq}+Cᵢᵥₚ, backtrack and combine the prevalence of the co-existence of 9-mer pairs in positions i−1 and i until the 1^{st} position. If there is no difference between two different connections, randomly pick one.

### HLA-Restricted Constructs

In some implementations, the vaccine construct (e.g., monovalent, bivalent or multivalent) may be designed to increase probability of binding to or presentation by a specific HLA allele or set of HLA alleles. In accordance with this implementation, the MHC binding affinities for each 9-mer in the multivalent (e.g., bivalent) polypeptide can be predicted using a tool such as NetMHC or MHCflurry, and 9-mers that do not bind with high affinity to a specific HLA allele of interest can be excluded. These tools are publicly available and are described, for example, in Lundegarrd et al. Nucleic Acids Res. 2008 Jul. 1; 36(Web Server issue):W509-12 and O'Donnell et al. Cell Systems 2018 7:129-132. Additional publicly available T-cell epitope prediction tools can be used in the herein described viral vaccine design methods and are described in, e.g., Sanchez-Trincado, et al., J Immunol Res (2017) 2017:2680160 (PMID: 29445754). Prediction tools for identifying MHC class I binding epitopes include, e.g., MAPPP, PEPVAC, EPISOPT, BIMAS, Propred-1, EpiJen, IEDB-MHCI, Net MHC, NetMHCpan, nHLApred, NetCTL and WAPP. Prediction tools for identifying MHC class II binding epitopes include, e.g., EpiDOCK, PREDIVAC, EpiTOP, TEPITOPE, Propred, IEDB-MHCII, IL4pred, MHC2PRED, NetMHCII and NetMHCIIpan. Prediction tools for identifying MHC class I and/or MHC class II binding epitopes include, e.g., MotifScan, Rankpep, SYFPEITHI, Vaxign, MHCPred, MULTIPRED2, SVMHC and SVRMHC.

In some implementations, the construct is further improved by performing a human proteome cross-recognition analysis, for example by a method comprising searching all of the 9-mers in the construct against a human proteome database such as UniProt to identify any 9-mers having a certain amino acid sequence identity with human peptides, e.g., at least 5 residues, or that share T cell receptor (TCR) facing residues with human proteins. Any such 9-mers may then be excluded from the construct. All remaining 9-mers are then combined, for example using a “beads on a string” approach, linking multiple epitopes in a single contiguous fusion polypeptide. See, e.g., Negahdaripour, et al., Infect Genet Evol. (2018) 58:96-109; Schubert, et al., Genome Med. 2016 Jan. 26; 8(1):9; Bounds, et al., Hum Vaccin Immunother. 2017 Dec. 2; 13(12):2824-2836; Toes, et al., Proc Natl Acad Sci USA. (1997) 94(26):14660-5; and Whitton, et al., J Virol. 1993 January; 67(1):348-52. In an alternate implementation, each of the remaining 9-mers is flanked with the most conserved 8 amino acid segments upstream and downstream to create 25 amino acid long peptides and all of the 25-mers are combined, for example using a “beads on a string” approach.

In some implementations, the polypeptide segments may optionally be rearranged to reduce or avoid deleterious junctional responses, for example by performing an HLA binding analysis, a human proteome cross-recognition analysis, or both, with respect to the junctional segments. Illustrative methods for reducing junction epitope presentation for neoantigens, in the context of designing anticancer vaccines, are described in WO 2019/104203.

In some implementations, the conserved regions are further defined by performing one or more of the following steps in silico: (i) removing short polypeptide segments, e.g., polypeptide segments of 35 or fewer amino acids in length, e.g., 9-35 amino acids in length; (ii) removing segments that are weakly immunogenic or non-immunogenic in humans; (iii) removing segments that are less than 90% conserved, in certain instances, less than 80% conserved, amongst a predetermined population of sequences; (iv) including additional segments from HIV-1 proteins, e.g., Env, Gag, Nef and Pol, that are known to be immunogenic in humans (see, e.g., epitope maps at hiv.lanl.gov/content/immunology/maps/maps.html; Fischer, et al., Nat Med. (2007) 13(1):100-6; and Addo, et al., J Virol, (2003) 77(3):2081-92).

In some implementations, adjacent polypeptide segments may optionally be separated with a linker sequence, as described above. In some implementations, the linker sequence or sequences comprise a cleavable linker, optionally further comprising an additional linker sequence located adjacent to the cleavable linker. The additional linker may be another cleavable linker, a polyalanine linker, a polyglycine linker, a flexible linker, or a rigid linker, as described above and herein. In some embodiments, a furin recognition site precedes or is positioned N-terminal to a 2A cleavable linker. In a specific implementation, where the linker sequence comprises a foot-and-mouth disease virus (FMDV) cleavable peptide, FMDV 2A, or derivative thereof, the additional linker sequence may be a REKR sequence (SEQ ID NO: 382), or derivative thereof. In some implementations, the linker is selected from a short polyalanine peptide, for example a peptide consisting of from 2 to 4 alanine residues (SEQ ID NO: 470), or having the sequence AAY (SEQ ID NO: 379) or AAX (SEQ ID NO: 380), where X is any amino acid residue.

In some implementations, the linker is inserted between each adjacent conserved region of the bivalent polypeptide. In some implementations, e.g., when no deleterious junctional epitope is created, no linker is inserted between adjacent segments of the same protein in the polypeptide. A linker can be inserted between adjacent segments of different proteins.

### Personalized Constructs

In some implementations, the disclosure further provides methods for producing a personalized construct. Generally, the personalized construct begins with a population-based vaccine construct that defines conserved regions and considers the sequence diversity of a subject's own viral isolates (DNA or RNA) in order to modify the segments of the reference population in order to generate sufficient T cell epitopes to cover the intra-patient viral diversity. Thus, the methods provide a final vaccine construct whose polypeptide sequence accounts for the viral diversity of the virus infecting a particular patient. To do this, the methods comprise analyzing individual patient deep sequencing data to define intra-patient viral sequence variability within each position of the conserved regions covered by the bivalent vaccine construct obtained using the methods described above. In essence, the method comprises identifying viral quasi-species variants existing within the subject and modifying the initial bivalent vaccine construct to reflect this diversity and further reduce the risk of escape mutants while improving for presentation by the individual's HLA alleles.

In an initial step, deep sequencing reads are assembled to create a subject-specific consensus sequence that is mapped to a reference sequence, such as HXB2. At each 9-mer position within the conserved regions covered by the corresponding sequences in the reference construct, i.e., the population construct prepared according to the methods described above, corresponding subsequences from a plurality of sequencing reads that completely covers that position are extracted and converted into 9 amino acid sequences (9-mers). Next, 9-mers in those positions that are covered by a sufficiently large number of sequencing reads, e.g., at least about 1000 reads, are extracted provide that they are present in at least a threshold proportion of the covered sequencing reads, e.g., at least about 1%. The extracted 9-mer subsequences are aligned to the sequences of the reference bivalent construct and mismatches are determined.

In some implementations, the method further comprises identifying regions that may serve as actual epitopes based on likelihood of presentation by the individual host's set of HLA alleles. The likelihood of binding to host HLA may be ascertained using predictive algorithms. Such algorithms have been designed, for example, using publicly available databases to develop deep learning models that model peptide binding per allele. This may be coupled with in-silico, published and/or experimental in-vitro T cell priming data that can define the potential impact of antigen variants in modulating T cell receptor (TCR) recognition or identify a peptide as an escape variant.

In some implementations, the method further comprises excluding sequences with pre-existing escape variants. For example, where an intra-patient 9-mer variant that is known or has been experimentally defined as an escape variant is also predicted to bind MHC, as determined using a tool such as NetMHC, NetMHCpan, MHCflurry, or other similar tools as discussed above, it may be excluded. In addition, any 9-mer variants that are not included in the bivalent vaccine construct, but map to a location covered by that construct, are identified and if the 9-mer variant is predicted to have low-binding affinity to the subject's HLA allele, the 9-mer location may need to be removed from bivalent vaccine construct in order to avoid escape. Possible escape variants for exclusion can also be determined, for example, using existing knowledge of escape variants, peptide prediction models, immunopeptidome analysis and in vitro T cell recognition data.

As discussed above, the method comprises analyzing viral proteome sequences obtained from an infected subject. In some implementations, the method may optionally further comprise a step of obtaining a sample of viral RNA or DNA from the subject. The sample may be plasma sample or a blood sample, for example a sample containing or enriched for peripheral blood mononuclear cells (PBMC) or lymphoid tissue. The sample may be obtained from the subject before anti-retroviral therapy (“preART”) or after ART. The sample may comprise one or more of plasma viral RNA, proviral DNA, intact proviral viral DNA, and reservoir outgrowth virus. In some implementations, the sample is obtained from HIV-infected treatment-naïve subjects. In some implementations, the sample is obtained from subjects following ART.

In some implementations, the methods may further comprise isolating viral RNA or DNA from a biological sample obtained from the subject, for example a serum sample, and sequencing the viral DNA.

The sequencing step may further comprise the assembly of a plurality of sequencing reads to create a subject consensus sequence; aligning each read in the plurality of reads to the subject consensus sequence; and mapping the aligned reads of the subject to a reference sequence to obtain sequence coordinates. The reference sequence may be, for example, the HIV reference sequence HXB2 identified by GenBank No. Accession K03455. Polypeptide sequences for HXB2 reference proteins Env, Gag, Nef and Pol are provided herein as SEQ ID NOs: 403-406.

### EXAMPLES

The following examples are offered to illustrate, but not to limit the claimed invention.

### Example 1

### Illustrated Implementation of the Conservation Analysis and Conserved Walking Analysis (CWA) to Generate a Bivalent Vaccine Construct

This Example describes the design of population-based bivalent polypeptide constructs by a specific implementation of the Conservation Analysis and CWA to generate a bivalent vaccine construct based on conserved protein regions encoded by the HIV-1 Env, Gag, Nef and/or Pol genes.

First, the method identifies a set of all conserved regions in a viral proteome for a selected set of viral genes. In this example, the set of viral genes consisted of HIV-1 Gag, Pol, and Nef.

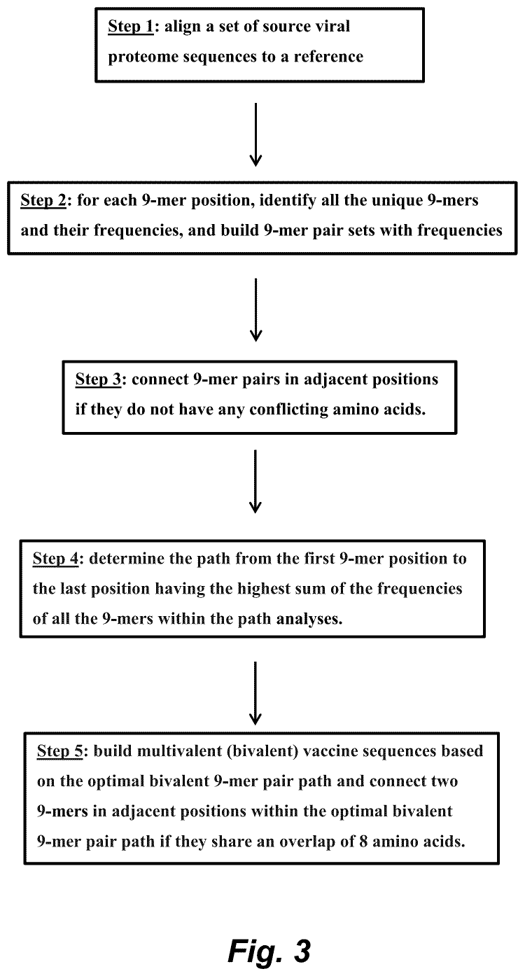

Computationally, the combination of the Conservation Algorithm and the CWA can be described as a positional De Brujin graph based bivalent vaccine sequence design algorithm comprising the following 5 basic steps, illustrated in FIG. 3:

### Step 1: Align a Set of Source Viral Proteome Sequences to a Reference Sequence.

In Step 1, a source population of viral proteome sequences is aligned to a reference sequence. In this example, the reference sequence used was the HIV-1 HXB2, identified by GenBank No. Accession K03455. The amino acid sequences of HXB2 reference polypeptides Env, Gag, Nef and Pol are provided herein as SEQ ID NOs: 403, 404, 405 and 406, respectively. The source population of viral proteome sequences consists of sequences obtained from naturally occurring viruses. Such sequences are publicly available, for example, from the HIV Databases maintained by the Los Alamos National Laboratory, the U.S. Dept. of Health and Human Services, and the National Institutes of Health (hiv.lanl.gov), which was the database used for the source population of sequences in this example. For the purposes of illustration, we focused our analysis on a subset of the viral sequences, here, sequences of Group M Clade B. The alignment was performed using a multiple alignment algorithm, specifically a fast Fourier transform algorithm, MAFFT. Katoh, et al. (2002) Nucleic Acids Res. 30 (14):3059-66. The base MAFFT software is publicly available and distributed, e.g., under the Berkeley Software Distribution (BSD) license.

Step 2: For Each 9-Mer Position, Pull Out all the Unique 9-Mers and their Frequencies, and Build 9-Mer Pair Sets with Frequencies.

In Step 2, we apply the Conservation Algorithm to the set of aligned sequences. For each sequence in the alignment, starting from the first amino acid of the N-terminus, the algorithm shifts one amino acid position at a time to create a set of all possible amino acid segments that are 9 amino acids in length, referred to as “9-mers.” The algorithm thus creates, for each sequence in the alignment, a set of 9-amino acid subsequences (“9-mers”) starting with the N-terminal amino acid, each subsequence overlapping the preceding subsequence by eight amino acids such that each sequence of length l in the alignment contains (l-8) 9-mers.

Next, for each 9-mer position, the method identifies the two most common unique 9-mers and their prevalence in the aligned set of source viral proteome sequences. Stated another way, starting at position i the two most common unique 9-mers at each position are identified based on their frequency, calculated as the number of times the unique 9-mer occurs at position i in the alignment divided by the total number of sequences in the alignment.

Computationally, each sequence of length l, contains l-8 9-mers. We define all the 9-mers starting at position i as sᵢⱼ and frequency as fᵢⱼ, j=1,2,3, . . . m. In total there are m unique 9-mers at position i. Each two unique 9-mers (sᵢᵤ, sᵢᵥ) can constitute a 9-mer pair and its frequency is fᵢᵤ+fᵢᵥ. And each 9-mer itself can constitute a 9-mer pair as (sᵢᵤ, sᵢᵤ) and its frequency is fᵢᵤ. Thus, in total, there are m*(m+1)/2 9-mer pairs at each position.

The method then calculates the bivalent conservation for each 9-mer position by summing up the proportions of aligned set of source viral proteome sequences containing either of the two most common 9-mers. To do this, a “bivalent conservation” is calculated for each position by summing the proportion of sequences in the alignment containing either of the two most common unique 9-mers.

Next, a new alignment of conserved regions is created by extracting the sequences in the alignment having a desired bivalent conservation. In this example, we used a bivalent conservation of greater than 80% or greater than 90%, meaning that the two most common 9-mers at position i account for more than 80% or more than 90% of the 9-mers at that position in the new alignment of conserved regions. Stated another way, the method identifies the conserved regions in the new alignment as those in which the sum of the frequencies of the two most common 9-mers at each position is greater than a certain cutoff, e.g., greater than 80% or greater than 90%. Thus, the method also calculates the frequency of each pair of unique 9-mers at each position in the new alignment of conserved regions.

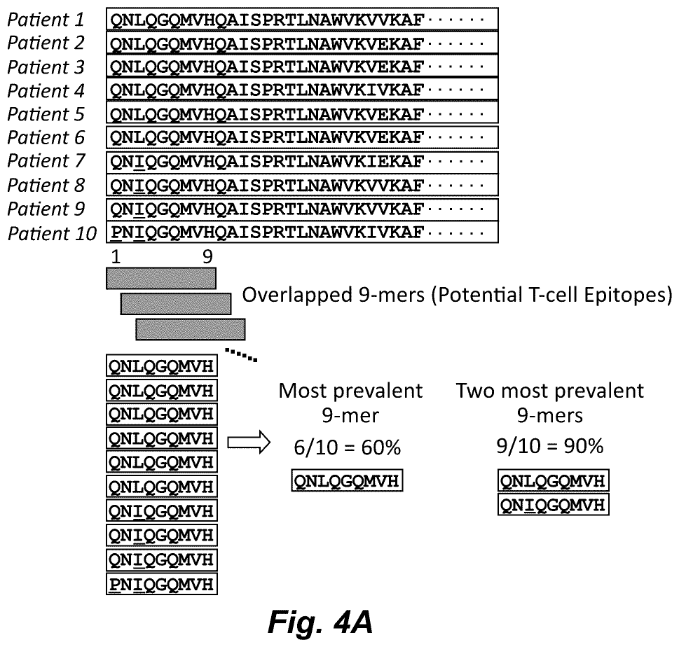

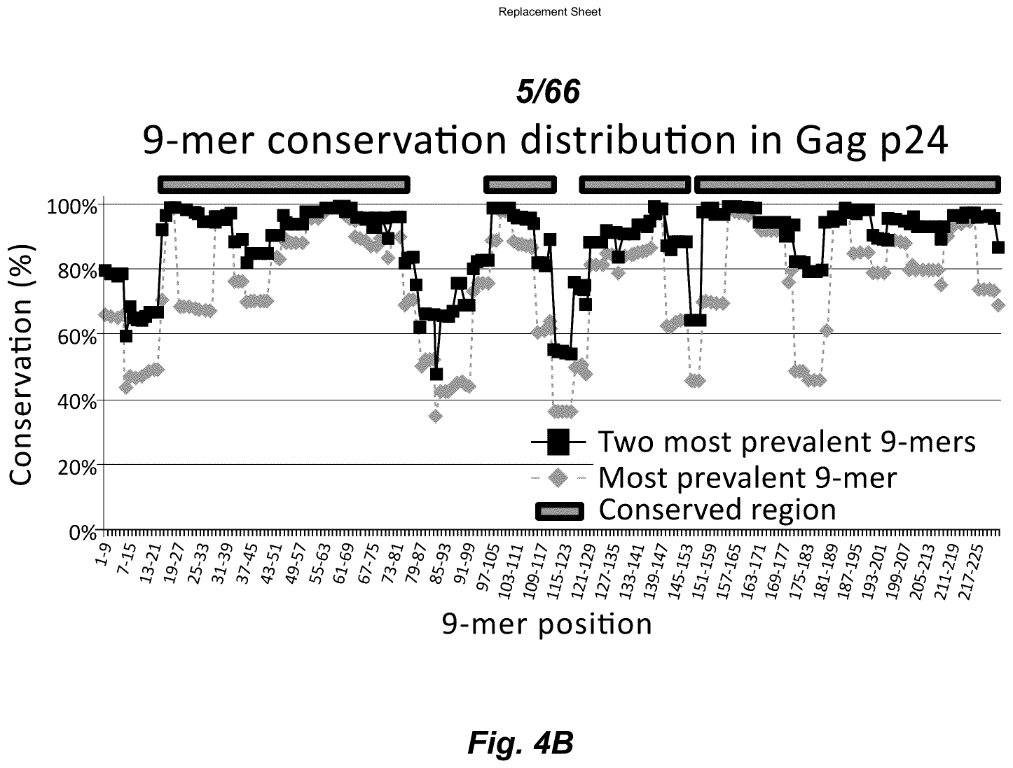

This is illustrated graphically in FIG. 4A. FIG. 4A shows a hypothetical set of 10 input natural sequences, each having a single amino acid variation within the first 9-mer. Across the set of 10 sequences, the 9-mer having an “L” at the third amino acid position occurs 6 times, the 9-mer having an “I” at that location occurs 3 times, and the 9-mer having an “I” at that location but a different amino acid in the first position occurs once. Thus, the Conservation Algorithm selects the two most prevalent 9-mers which together account for 90% of the possible 9-mers at that position in the population of aligned sequences.

Using this analysis, the distribution of highly conserved 9-mers at each position across all of the protein sequences in the population can be determined. This is illustrated graphically in FIG. 4B. The plot shows the conservation distribution for proteins encoded by the Gag gene p24 protein in 9,846 Group M Clade B input sequences obtained from the Los Alamos HIV Sequence database. The y-axis shows bivalent conservation and the x-axis shows the location of the 9-mer relative to the reference sequence, Gag p24 from HXB2. Across the top of the graph the horizontal bars depict conserved regions as those having at least 80% bivalent conservation using the two most prevalent 9-mers at each position. The dark gray line with the squares plots the bivalent conservation at each position using the two most prevalent 9-mers while the light gray line with the diamonds shows conservation using only the most prevalent 9-mer at each position. This analysis demonstrates that the use of the two most prevalent 9-mers improves the identification of structurally conserved sequences with an input population.

We next applied further selection criteria to define the conserved regions, including restricting to regions having greater than 90% bivalent conservation and removing short segments of less than 35 amino acids, e.g., segments 9-35 amino acids in length.

We also included some additional segments from certain regions having at least 80% bivalent conservation and known to be highly immunogenic, in particular, the region of Nef corresponding to amino acids 64-99 of the reference sequence HXB2_K03455 (see, e.g., epitope maps at hiv.lanl.gov/content/immunology/maps/maps.html; Fischer, et al., Nat Med. (2007) 13(1):100-6; and Addo, et al., J Virol, (2003) 77(3):2081-92).

Step 3: Connect 9-Mer Pairs in Adjacent Positions if they do not have any Conflicting Amino Acids.

Using this modified set of conserved regions, we applied the CWA to build bivalent sequence constructs. The CWA connects 9-mer pairs in adjacent positions of the alignment of conserved regions that share an overlap of eight amino acids.

Computationally, each 9-mer s contains 9 amino acids, we write s[x:y] to represent the amino acid subsequence from position x to y, y−x+1 amino acids in total:

sᵢᵤ[2:9]=sᵢ₊₁ₚ[1:8] and sᵢᵥ[2:9]=s_{i+1q}[1:8]

or

sᵢᵤ[2:9]=s_{i+1q}[1:8] and sᵢᵥ[2:9]=sᵢ₊₁ₚ[1:8].

Step 4: Find the Optimal Path from the First 9-Mer Position to the Last Position in Terms of the Sum of the Frequencies of all the 9-Mers within the Path.

In Step 4, the algorithm builds a directed acyclic graph in which each 9-mer pair is a node and the edges between adjacent nodes are formed from the connected 9-mer pairs in the adjacent positions with the weight of each edge equal to the frequency of the downstream 9-mer pair. This directed acyclic graph is a positional De Brujin graph. Such graphs have been described in connection with assemblies of next generation sequencing data, for example as described in Ronen et al., Bioinformatics (2012) 28:188-196.

In the present example, we add a source node and connect it with all of the nodes in the first position; and we add a sink node and connect it with all of the nodes in the last position. In a directed graph, a source node is a node that only has out flow and a sink node is a node that only has in flow. Here, the source node is a dummy node that connects to all the 9-mer pair nodes in the first position, and the sink node is a dummy node that connects to all the 9-mer pair nodes in the last position.

We then negate all of the weights and find the optimal path from the source node to the sink node, where the optimal path is defined in terms of the sum of the frequencies of all 9-mer pairs. The task of finding the optimal path is performed, for example, using the Bellman-Ford algorithm. Generally, the Bellman-Ford algorithm computes the shortest paths from a single source vertex to all of the other vertices in a weighted directed graph. A directed graph is one made up of a set of vertices connected by edges, where the edges have a direction associated with them.

Computationally, the basic idea is to model the maximum coverage bivalent vaccine construction problem as a classic graph theory problem where the solution is finding the minimum path in a directed acyclic graph. The computational steps can be summarized as follows:
(4-1) Treat each 9-mer pair as a node, and build edges between adjacent nodes in Step 3;
(4-2) Adding a source node and connect it with all the nodes at the 1st position;
(4-3) Adding a sink node and connect it with all the nodes at the last position;
(4-4) Weight of each edge equals to the frequency of downstream 9-mer pair; and
(4-5) Negating all the weights and finding the optimal path using the Bellman-Ford algorithm.
   Step 5: Build Bivalent Vaccine Sequences Based on the Optimal Bivalent 9-Mer Pair Path and Connect Two 9-Mers in Adjacent Positions within the Optimal Bivalent 9-Mer Pair Path if they Share an Overlap of 8 Amino Acids.

In Step 5, a bivalent construct is built by connecting two 9-mers in adjacent positions within the optimal bivalent 9-mer path if they share an overlap of eight amino acids, thereby creating two sequences of connected 9-mers which together form the bivalent construct. The connected adjacent 9-mer pairs all have an 8 amino acid overlap, so they will be assembled into two sequences. For example, one 9-mer pair (AIIIIIIIS (SEQ ID NO: 464), MIIIIIIII (SEQ ID NO: 465) ) can be connected with another 9-mer pair (IIIIIIISK (SEQ ID NO: 466), IIIIIIIIR (SEQ ID NO: 467) ) and make two sequences (bivalent sequences): AIIIIIIISK (SEQ ID NO: 468) and MIIIIIIIIR (SEQ ID NO: 469).

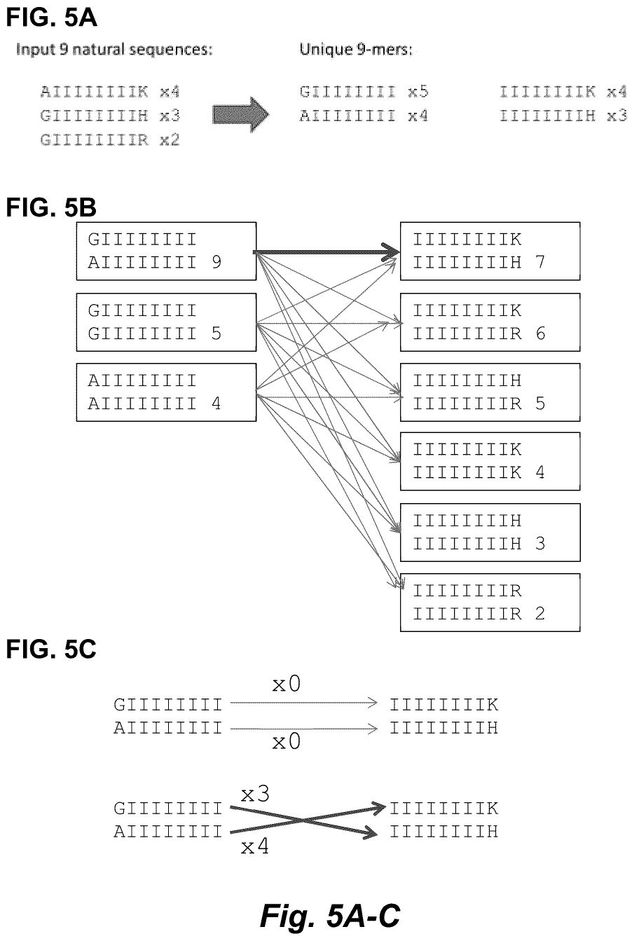

This method is illustrated graphically in FIGS. 5A-5C. FIG. 5A shows a hypothetical set of 9 source viral sequences having, at the first position, 2 unique 9-mers and at the second adjacent position 3 unique 9-mers. The frequency of each sequence is indicated to the right of the sequence as ‘times’, e.g., “x5” means that sequence occurs 5 times in the source set. FIG. 5B depicts the building of the positional De Brujin graph in which each node is one bivalent 9-mer pair. Where two bivalent 9-mer pairs in adjacent positions share an overlap of eight amino acids they are connected to build an edge. In this manner the directed acyclic graph is created. FIG. 5C illustrates the finding of the optimal path. As noted above, the optimal path is defined in terms of the sum of the frequencies of all 9-mer pairs. This is accomplished by finding the connection between adjacent 9-mers that provides the highest conservation with reference to the input sequences. Thus, in FIG. 5C, connecting the two 9-mer pairs as shown in the top set of four pairs provides the following bivalent sequences,

(SEQ ID NO: 471)
GIIIIIIIIK
x0
(SEQ ID NO: 472)
AIIIIIIIIH
x0.

Neither of these sequences is present in the source sequences shown in FIG. 5A.

In contrast, connecting the two 9-mer pairs as shown in the bottom set of four pairs in FIG. 5C provides the following bivalent sequences,

(SEQ ID NO: 473)
GIIIIIIIIH
x3
(SEQ ID NO: 474)
AIIIIIIIIK
x4.

Each of these is present, 3 or 4 times, respectively, in the source sequences shown in FIG. 5A. Accordingly, it is these second pair of bivalent sequences that is selected by the algorithm because it maximizes conservation relative to the source sequences.

Computationally, this can be illustrated by the following exemplary cases:
Case 1: if sᵢᵤ[2:9]=sᵢ₊₁ₚ[1:8] and sᵢᵥ[2:9]=s_{i+1q}[1:8], connect sᵢᵤ with sᵢ₊₁ₚ and sᵢᵥ with s_{i+1q};
Case 2: if sᵢᵤ[2:9]=s_{i+1q}[1:8] and sᵢᵥ[2:9]=sᵢ₊₁ₚ[1:8], connect sᵢᵤ with s_{i+1q}and sᵢᵥ with sᵢ₊₁ₚ;
Case 3: if sᵢᵤ[2:9]=sᵢ₊₁ₚ[1:8] and sᵢᵥ[2:9]=s_{i+1q}[1:8] and sᵢᵤ[2:9]=s_{i+1q}[1:8] and sᵢᵥ[2:9]=sᵢ₊₁ₚ[1:8], the selection of connection is based on the prevalence of the two connections in natural sequences:

Denote the prevalence of the co-existence of sᵢₓ and s_{i+1y} in input sequences as C_{ixy};
If Cᵢᵤₚ+C_{ivq}>C_{iuq}+Cᵢᵥₚ, connect sᵢᵤ with sᵢ₊₁ₚ and sᵢᵥ with s_{i+1q};
If C_{iuq}+Cᵢᵥₚ>Cᵢᵤₚ+C_{ivq}, connect sᵢᵤ with s_{i+1q} and sᵢᵥ with sᵢ₊₁ₚ;
If Cᵢᵤₚ+C_{ivq}=C_{iuq}+Cᵢᵥₚ, backtrack and combine the prevalence of the co-existence of 9-mer pairs in positions i−1 and i until the first position. If there is no difference between two different connections, randomly pick one.

This backtrack and co-existence prevalence approach considers prevalence of peptides longer than 9 amino acids and further differentiates the present algorithm from other graph-based methods.

Next, constructed sequences from regions not adjacent to one another in the natural sequence, that is, regions which could not be joined according to the CWA as described above due to their lacking an 8 amino acid overlap, were combined using one of three different linker strategies: 1. direct fusion without any linker; 2. insert ‘AAA’ linker (SEQ ID NO: 378) between each two conserved regions; 3. direct fusion without any linker for segments within the same protein and insertion of an F2A linker between segments from different proteins.

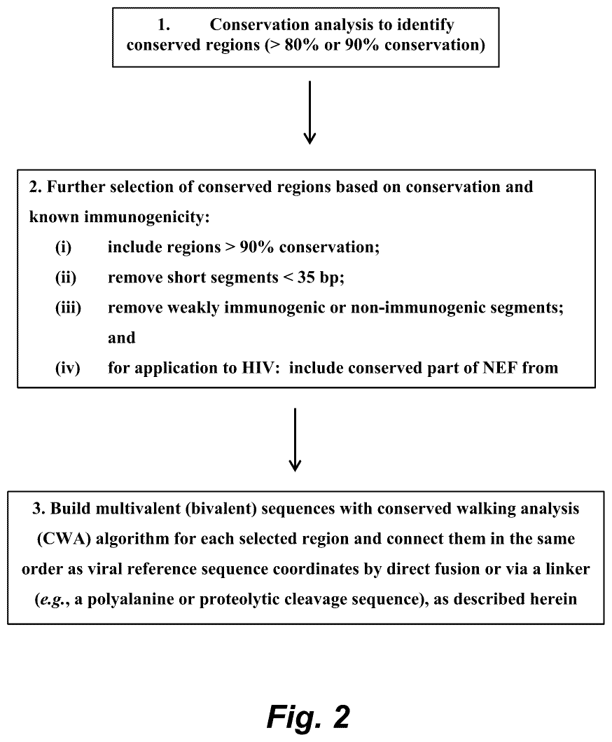

An overview of the Conserved Walking Analysis (CWA) method is shown in FIGS. 1 and 2. The fusion polypeptides of SEQ ID NOs: 345-350 and the sequences in Table 1, which have polypeptide segments encoding by the HIV-1 Gag, Nef and Pol genes, are exemplary immunogenic fusion polypeptide sequences designed according to this method.

### Example 2

### Illustrated Implementation of the Conservation Analysis and Conserved Walking Analysis (CWA) Applied to Proteins Encoded by HIV-1 Genes

This example describes a similar implementation based on conserved HIV-1 regions of (i) Gag and Nef (“GagNef”), (ii) Pol, or (iii) Pol and Env (“PolEnv”).

In Example 1 above, the Conservation algorithm was applied to identify a set of all candidate conserved regions in the protein coding regions of the target genes Gag, Nef, Env and Pol. In this example, we utilized the protein coding regions of (1) Gag and Nef, (2) Pol or (3) Pol and Env to generate three different bivalent constructs, “GagNef,” “Pol” and “PolEnv,” respectively. As in Steps 1-2 of Example 1 above, we first aligned the source sequences and then applied the Conservation Algorithm to identify a set of all candidate conserved regions in the protein coding regions of the target genes, which were either Gag and Nef, Pol, or Pol and Env. As above, we then we applied further selection criteria based on conservation and known immunogenicity (see, e.g., epitope maps at hiv.lanl.gov/content/immunology/maps/maps.html and Fischer, et al., Nat Med. (2007) 13(1):100-6). In certain sequences including polypeptide segments encoding by the Pol gene, we excluded sequence segments including one or both of the “YMDD” motif (SEQ ID NO: 462) in reverse transcriptase and the “DTG” motif in protease, because they may affect expression the maintenance of enzymatic activity.

Using this modified set of conserved regions, we applied the CWA to build bivalent sequence constructs, as in Steps 3-5 in Example 1.

Some polypeptide segments were connected by a polyalanine linker (e.g., AA, AAA (SEQ ID NO: 378) or AAY (SEQ ID NO: 379)), chosen for demonstration purposes because it is a small flexible linker that is unlikely to have a significant influence on protein structure. If we determined that it was possible to fuse polypeptide segments without creating a deleterious or undesirable junctional epitope, e.g., such as one that may stimulate T cells that may cross react to self-antigens, a fusion approach was used. If we determined that a deleterious or undesirable junctional epitope may be created, a flexible linker was inserted between polypeptide segments.

For this Example, we applied a further analysis of the junctional regions for possible presentation of deleterious epitopes and arranged the segments to reduce or avoid the creation of such junctional epitopes.

Different arrangements of peptide segments generate different junction 9-mers that can induce different junction responses. We developed a polypeptide segment arrangement tool to examine MHC binding affinities and cross-recognition with human peptides for all the junction 9-mers in each arrangement. Our internally developed polypeptide segment arrangement tool searches different arrangements of peptides and determines the best arrangement with minimal junction response based on in silico prediction results of applying the two analyses described below ((1) in-silico HLA binding analysis and (2) human proteome analysis to identify epitopes that may prime T cells that may recognize self-antigens) on the junctions of 9-mers. The junctional response score between each two adjacent segments is determined by the sum of the number of junction 9-mers that are predicted to have high binding affinities to target HLA alleles and the number of human proteins predicted to have peptides or T cell recognition motifs with any junction 9-mers. The score of each segment arrangement is determined by the sum of the junctional response scores for all the junctional regions in each segment arrangement.
1) When there are less than 15 peptide segments, our internally developed polypeptide segment arrangement tool searches all the possible arrangements and determines the best one with minimal junction response (the lowest segment arrangement score)
2) When there are at least 15 peptide segments, our internally developed polypeptide segment arrangement tool uses a ‘greedy’ strategy. It first creates all the junctions and then starts from the best junction in terms of predicted junctional response. Next, it searches for the next compatible best junction iteratively and assembles all the peptide segments.

In-silico MIC class I (human HLA) binding analysis: Antigen processing, presentation, and T cell receptor recognition are complex processes that remain incompletely understood. Intracellular and extracellular antigens are processed within endosomal compartments, and the cytoplasm by the proteasome and trafficked to endosomal compartments such as the ER where they peptide fragments interact with MHC molecules. Stable peptide-MHC complexes are trafficked to the cell surface where they can be recognized by a T cell expressing a TCR with the appropriate specificity. One of the most selective steps in antigen processing and presentation is HLA binding. HLA binding affinities can be predicted using various tools such as NetMHC or MHCflurry, or large internal datasets derived from immunopeptidome analyses and confirmed by experimental binding data as well as epitopes defined from patient samples. These tools are publicly available and are described, for example, in Lundegarrd et al., Nucleic Acids Res. 2008 Jul. 1; 36(Web Server issue):W509-12 and O'Donnell, et al., Cell Systems 2018 7:129-132. In this example we used NetMHC. The default settings were used for all the parameters in NetMHC, along with inputting information for peptide sequences and HLA alleles. Predicted binding affinities with an IC50 value less than 1,000 nM are considered as low binding affinities.

Human proteome cross-recognition analysis: Epitopes similar to human peptides may induce tolerogenic responses or responses that may cross-react with self-antigens. We searched all the 9-mers in our vaccine against public human protein databases (e.g., Uniprot, NCBI). If an HIV peptide 9-mer has at least a 5-residue amino acid sequence identity with a human peptide 9-mer, and both are predicted to have high binding affinities to the same alleles, they are considered as cross-conserved 9-mers. We downloaded all the human protein sequences from the UniProt database and built a tool to support efficient search of a given 9-mer against all the human protein 9-mers with up to 4 mismatches (at least 5 matches).

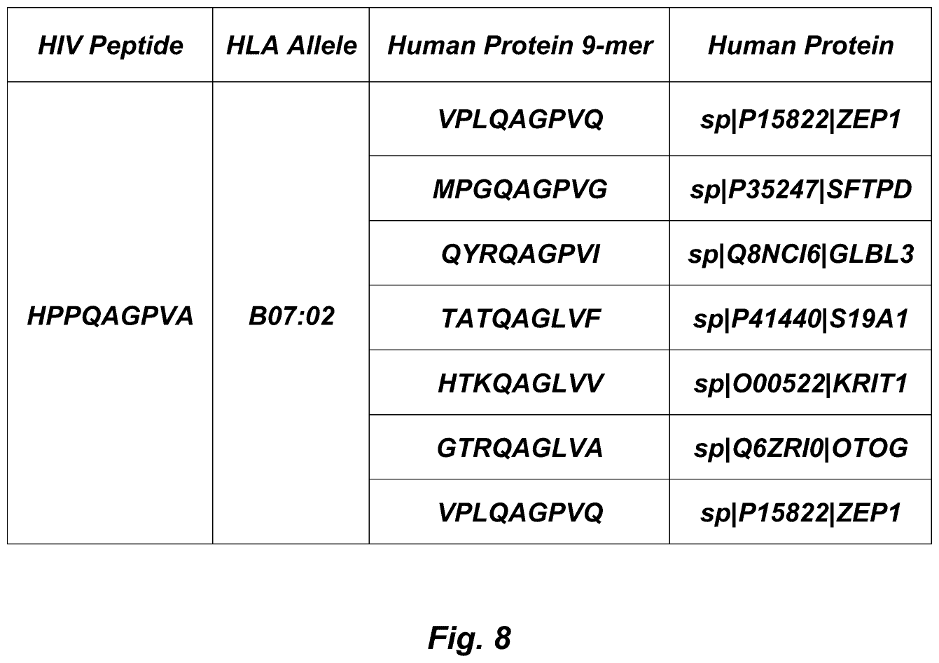

FIG. 8 illustrates the results of human proteome cross-recognition analysis. In this example, we searched HIV-1 peptide 9-mers over human protein databases and identified all the human protein 9-mers sharing a certain number of amino acids (at least 5 tentatively) and are predicted to have high binding affinities (e.g., IC50 of less than about 1000 nM or having a percentile rank within the top 5% in a population of polypeptide segments) to the same alleles based on the in silico MHC class I analysis described herein. Such HIV 9-mers having both high sequence identity (e.g., having have at least 55% (5 of 9 amino acid residues), e.g., at least 65% (6 of 9 amino acid residues), e.g., at least 75% (7 of 9 amino acid residues), e.g., at least 85% (8 of 9 amino acid residues)) to a peptide segment of a human protein and high predicted MHC class I binding affinity are excluded because they may induce tolerogenic responses or responses that may cross-react with human self-antigens (defined herein as “deleterious epitopes.”).

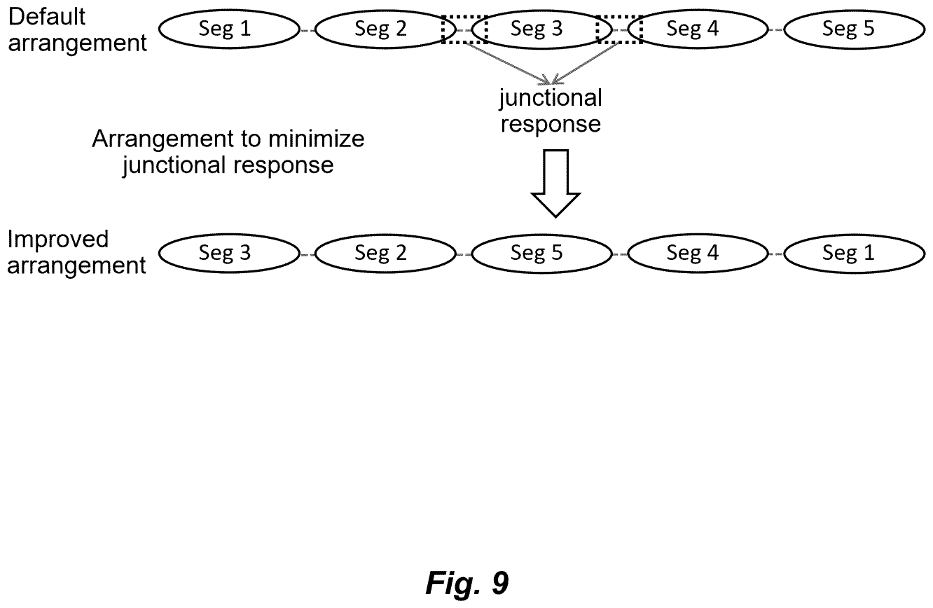

FIG. 9 illustrates how polypeptide segment arrangement analysis can reduce or eliminate possible presentation of deleterious or undesirable epitopes injunction regions. In the illustrated default arrangement, the junction 9-mers between Seg 2 and Seg3, and between Seg 3 and Seg 4 are predicted to produce junctional sequences that may induce tolerogenic or self-reactive responses in a human (e.g., having either high MHC binding affinity based on in silico HLA binding analysis or cross-recognition with human proteins based on human proteome cross-recognition analysis). We applied an algorithm that searches different arrangements and determines an arrangement that results in reduced or eliminated predicted junctional sequences that may induce tolerogenic or self-reactive responses in a human.

The fusion polypeptides of SEQ ID NOs: 351-366 and 407-410 are exemplary immunogenic fusion polypeptide sequences designed according to this method.

### Example 3

### MHC Class I Restricted Fusion Polypeptides

A component of improving the design of antigens for T cell vaccines is to define a desirable set of antigens that can be readily presented by the hosts' T cells and prime a T cell response. Short amino acid fragments (8-30aa long), derived from viral antigens are processed and presented on host Human Leukocyte Antigen (HLA) alleles that are defined within the Major Histocompatibility Complex (MHC). These alleles are defined as MHC class I if they present peptides that are recognized by the T cell receptor (TCR) on CD8+ T cells, and MHC class II if the peptide and MHC complex is recognized by TCRs on CD4+ T cells.

This example describes an approach in which a set of MHC class I restricted 9-mers is selected from the bivalent constructs and combined to form a MHC class I restricted vaccine construct. This method is illustrated by designing an immunogenic fusion polypeptide with multiple epitopes predicted to bind to human HLA-A*0201 allele. We selected the human HLA-A*0201 allele to demonstrate the method because it is a very common allele in the United States.

Two approaches were used to generate HLA-A*0201 restricted sequences, a “short peptide” approach and a “long peptide” approach. For the short peptide approach, we applied the in-silico MHC class I binding analysis described in Example 2 to identify any 9-mers in the bivalent sequences that were predicted to have low binding affinity to HLA-A*0201. Low affinity 9-mers (e.g., 9-mers having a predicted MHC class I binding IC50 value of less than 1,000 nM) were removed from the constructs.

Next, as described in Example 2, we performed a human proteome cross-recognition analysis for all the 9-mers in the bivalent construct. We identified any 9-mers sharing at least 5 residues with human peptide sequences and removed them from the constructs.

We subsequently applied our internally developed polypeptide segment arrangement tool described in Example 2 and combined all of the remaining 9-mers in both of the bivalent sequences into a single sequence, arranged in an order to reduce or avoid undesirable junctions. We refer to this as a “beads on a string” approach. In a vaccine construct that is based on single or multiple MHC class I allele binding specificities, induction of a helper CD4+ T cell responses can be achieved by including MHC class II epitopes. These may be class II epitopes defined in the literature and known to be targeted by a large proportion of the population or may be tailored to the individuals own MHC class II alleles (Ranasinghe, J Virol, (2012) 86(1):277-83; and Kaufmann, et al., J Virol. (2004) 78(9):4463-77).

For the “long peptide” approach, we performed the same steps as described above to arrive at the “short peptide” sequence, except after removing the low affinity MHC class I (here, human HLA-A*0201) binding 9-mers, each of the remaining 9-mers was flanked with the most conserved 8 amino acid segments upstream and downstream to create 25 amino acid long peptides. Then, as with the short peptide approach, all of the 25-mers are combined into a single sequence using a “beads on a string” approach, arranged in an order to reduce or avoid undesirable junctions.

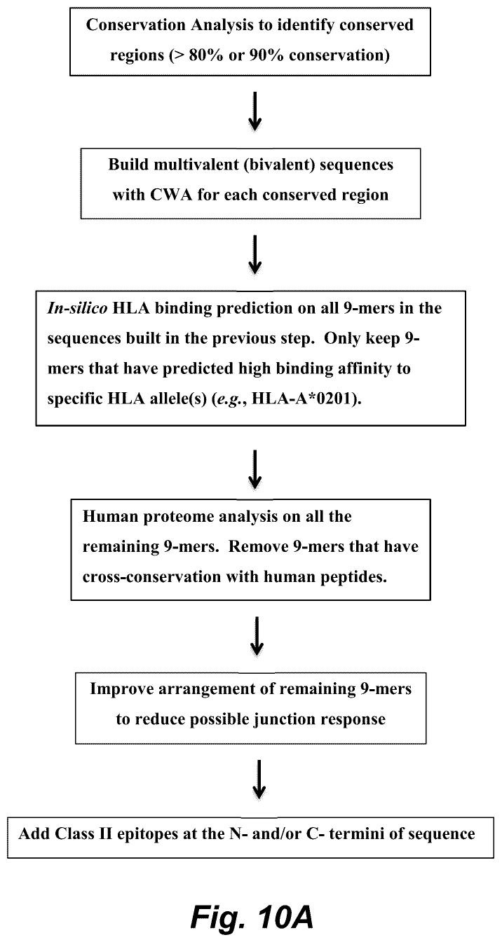

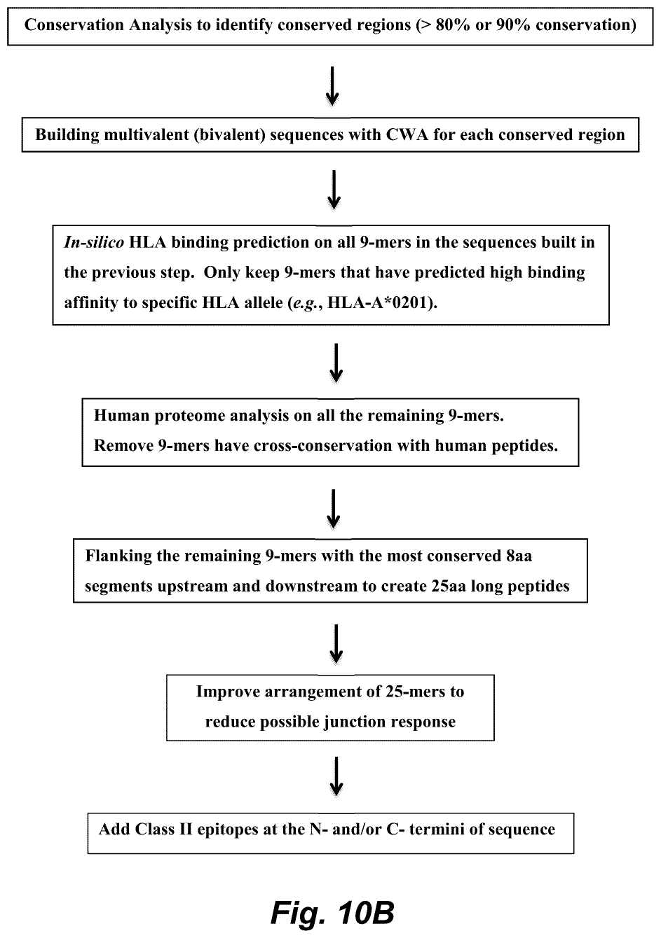

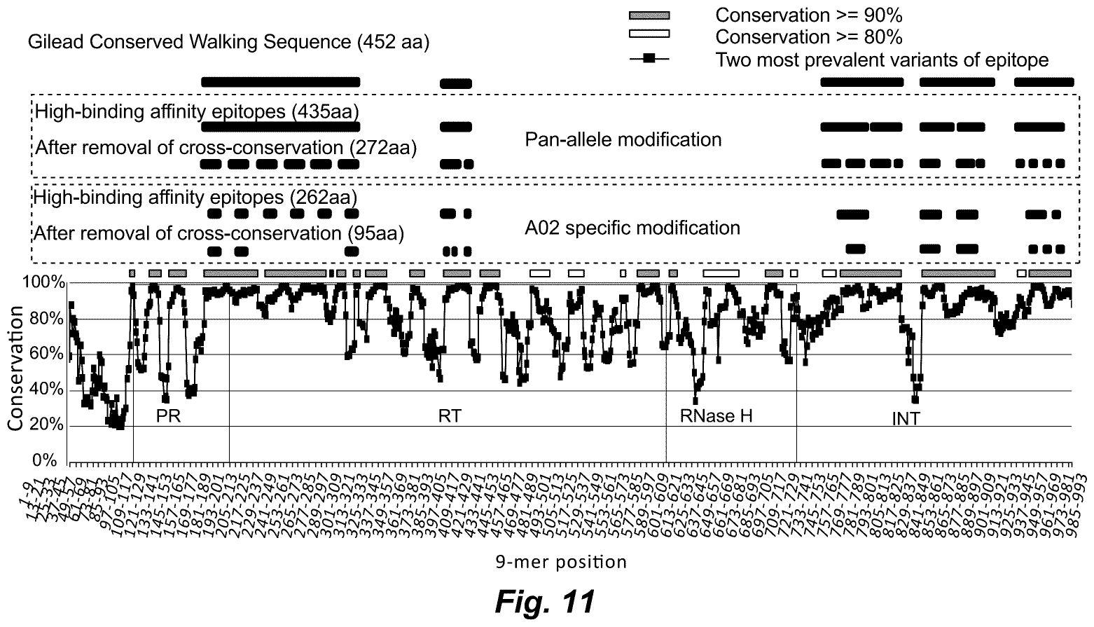

FIG. 10A-B provides a flow diagram illustrating the basic methodology of the “short peptide” and “long peptide” approaches, respectively. The fusion polypeptides of SEQ ID NOs: 367-377 and 411 are exemplary immunogenic fusion polypeptide sequences designed according to this method.

### Example 4

### Individualized Construct with Deep Sequencing Data Analysis Incorporated

This example describes an approach in which deep sequencing data analysis is included to form an individualized vaccine construct. In this Example, as in Steps 1-2 of Example 1 above, we first aligned the source sequences and then applied the Conservation Algorithm to identify a set of all candidate conserved regions in the protein coding regions of the target genes. In this example, the target genes were Gag, Nef and Pol. We applied the CWA to build bivalent sequences in those regions, as in Steps 3-5 of Example 1.

In addition to the 9-mers derived from downloaded population sequences, we also analyzed deep sequencing data of the target individual to identify intra-patient diversity within those conserved regions. To identify intra-patient 9-mer variants using deep sequencing data, deep sequencing reads were assembled to create subject-specific consensus sequences. The deep sequencing reads were aligned to subject-specific consensus sequence and then alignment was mapped to HXB2 position coordinates based on alignment of subject-specific consensus to HXB2 reference sequence. At each 9-mer position within the conserved regions, corresponding subsequences from all the sequencing reads completely covering that position were extracted converted into 9 amino acid sequences. Only 9-mer variants with prevalence exceeding assay background were included.

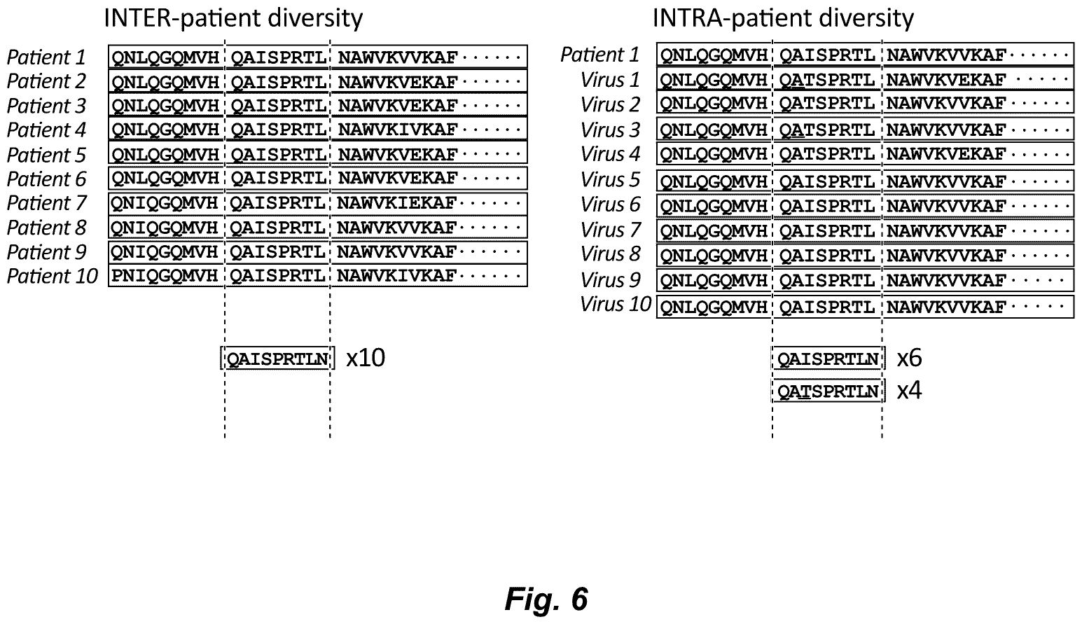

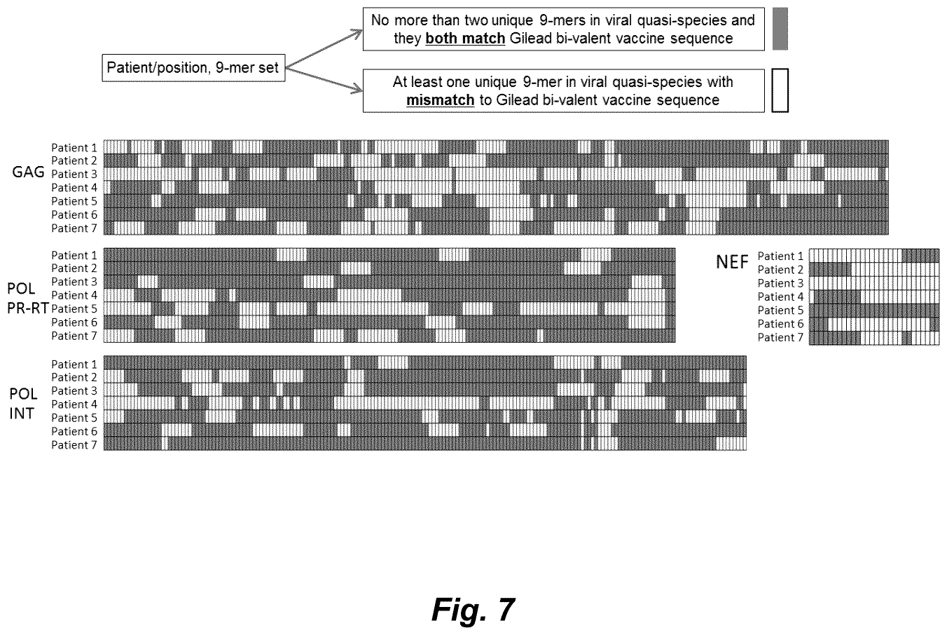

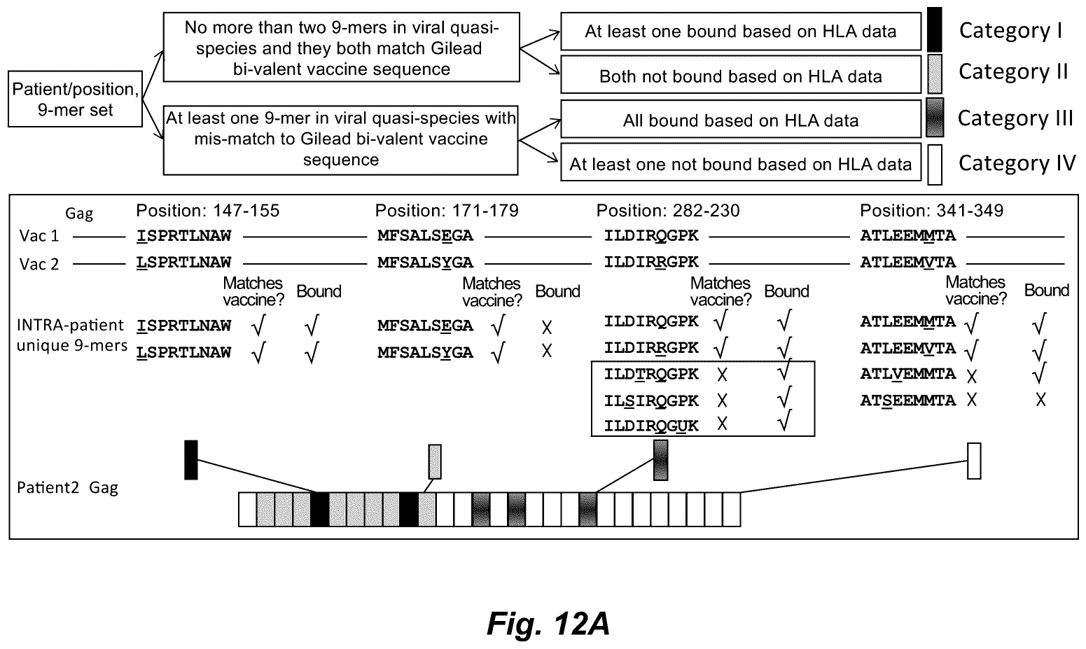

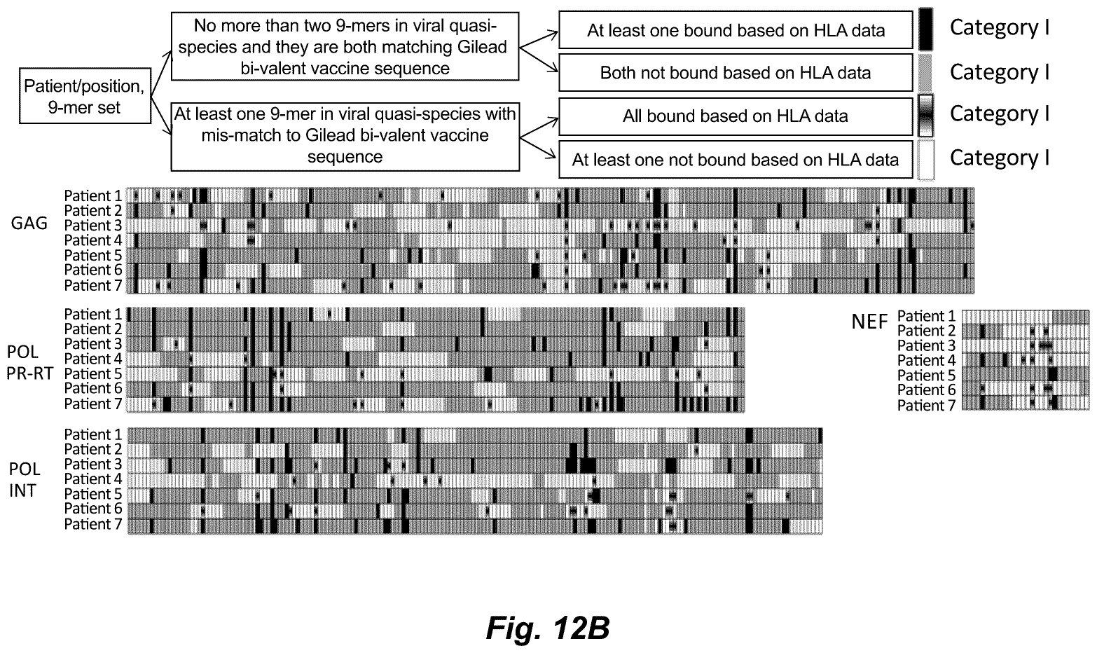

For all the 9-mers position within conserved regions, we performed the in-silico HLA prediction analysis and then classified all the positions into four categories (FIG. 6). Positions are classified into Category I (represented by [symbol] in FIG. 12A) if all the intra-patient 9-mers match bivalent sequences and at least one of them have high predicted binding affinities. Positions are classified into Category II (represented by [symbol] in FIG. 12A) if all the intra-patient 9-mers match bivalent sequences and all of them have low predicted binding affinities. Positions are classified into Category III (represented by [symbol] in FIG. 12A) if at least one of the intra-patient 9-mers does match bivalent sequences and all of them have high predicted binding affinities. Positions are classified into Category IV (represented by [symbol] in FIG. 12A) if at least one of the intra-patient 9-mers does match bivalent sequences and at least one of them has low predicted binding affinity.

Next, based on the conserved region position classification results, all the 9-mer variants in Category IV positions are removed and only 9-mers that have high binding affinity (e.g., 9-mers having a predicted MHC class I binding IC50 value of greater than 1,000 nM) to patient HLA allele in other position are kept. Any epitopes that are known to be escape variants (i.e., sequence variants that escape T cell recognition) based on external public HIV databases (hiv.lanl.gov) or internal experimental data are removed. As described in Example 2, we performed a human proteome cross-recognition analysis for all the remaining 9-mers and any 9-mers sharing at least 5 residues with human peptide sequences are removed. Then as described in the “long peptide” approach in Example 3, each of the remaining 9-mers was flanked with the most conserved 8 amino acid segments upstream and downstream to create 25 amino acid long peptides (25-mers). In a final step, we applied our internally developed polypeptide segment arrangement tool described in Example 2 and combined all of the 25-mers into a single sequence (“beads on a string” approach).

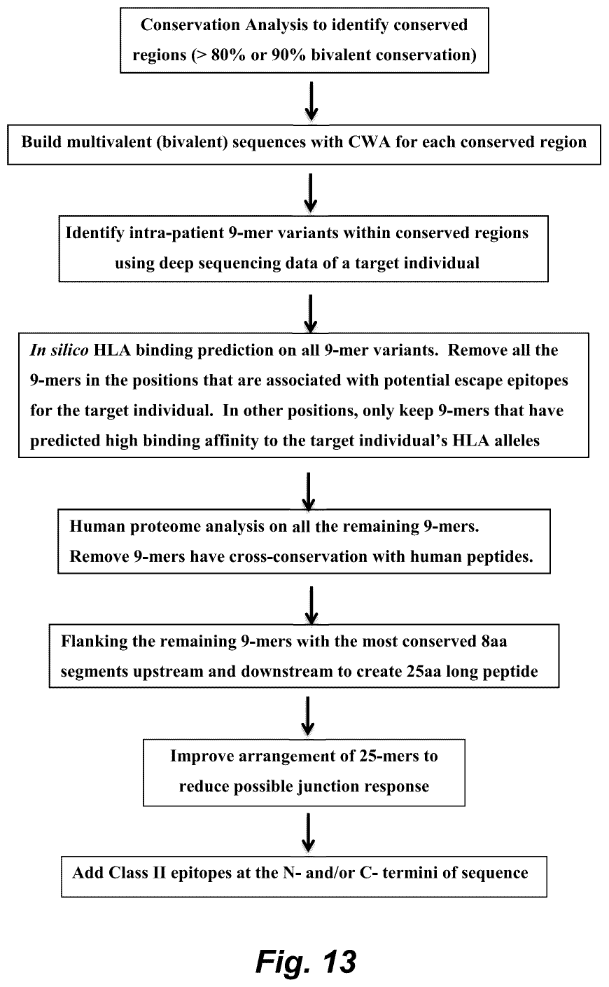

FIG. 13 provides a flow diagram illustrating the basic methodology of the individualized construct approach. SEQ ID NO: 422 provides an exemplary immunogenic fusion polypeptide sequence designed according to this method. It is an illustrative individualized construct designed with deep sequencing data analysis incorporated; generated using deep sequencing data from a patient with HLA alleles: A*02:01, A*23:01, B*07:02, B*44:03, C*04:01, and C*07:02.

### Example 5

### HLA Restricted Construct Improved with Deep Sequencing Data Analysis

This example describes an approach in which deep sequencing data and patient HLA data analyses are included to further improve the HLA restricted vaccine construct described in Example 3. In this Example, as in Steps 1-2 of Example 1 above, we first aligned the source sequences and then applied the Conservation Algorithm to identify a set of all candidate conserved regions in the protein coding regions of the target genes. In this example, the target genes were Gag, Pol and Nef. We applied the CWA to build bivalent sequences in those regions, as in Steps 3-5 of Example 1.

In addition to the 9-mers derived from downloaded population sequences, we also analyzed deep sequencing data of four individuals with the same HLA allele (HLA-A*0201) to identify intra-patient diversity within those conserved regions.

As described in Example 4 above, we analyzed deep sequencing data and classified all the conserved region positions into four categories for each individual.

For each of the positions in conserved regions, if it is in Category IV for at least one patient, all the 9-mer variants are removed as this indicates that an escape pathway for that allele has been defined. While in all the other positions, only 9-mers that have high binding affinity to the target HLA allele (HLA-A*0201 in this example) are kept. As described in Example 2, we performed a human proteome cross-recognition analysis for all the remaining 9-mers and any 9-mers sharing at least 5 residues with human peptide sequences are removed. Then as described in the “long peptide” approach in Example 3, each of the remaining 9-mers was flanked with the most conserved 8 amino acid segments upstream and downstream to create 25 amino acid long peptides (25-mers) (see, Assadipour, et al., Clin Cancer Res. (2017) 23(15):4347-4353; Zhang, et al., J Biol Chem, (2009) 284(14):9184-91). In a final step, we applied our internally developed polypeptide segment arrangement tool described in Example 2 and combined all of the 25-mers into a single sequence (“beads on a string” approach).

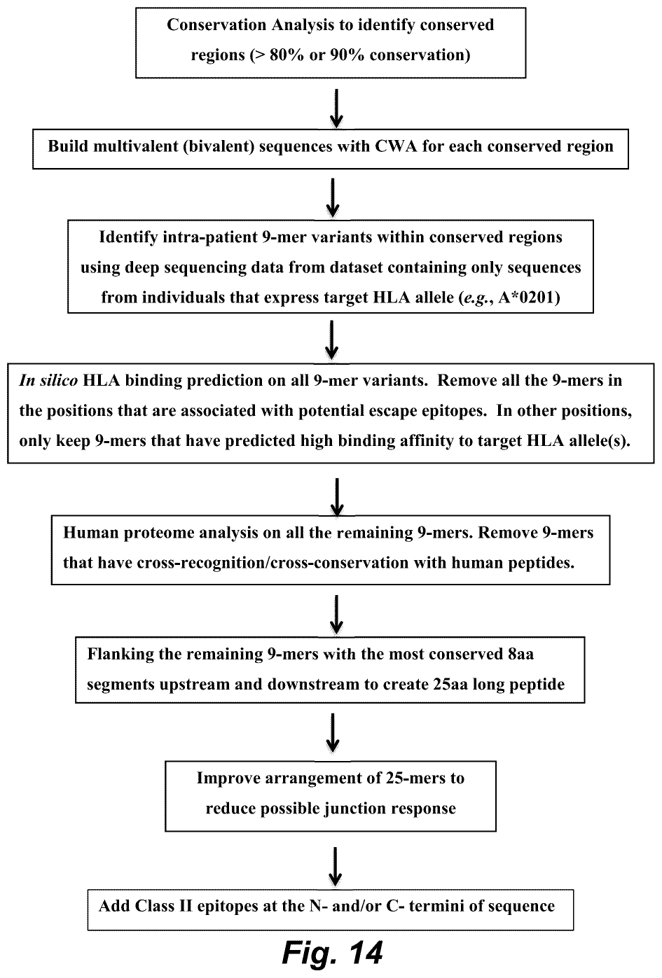

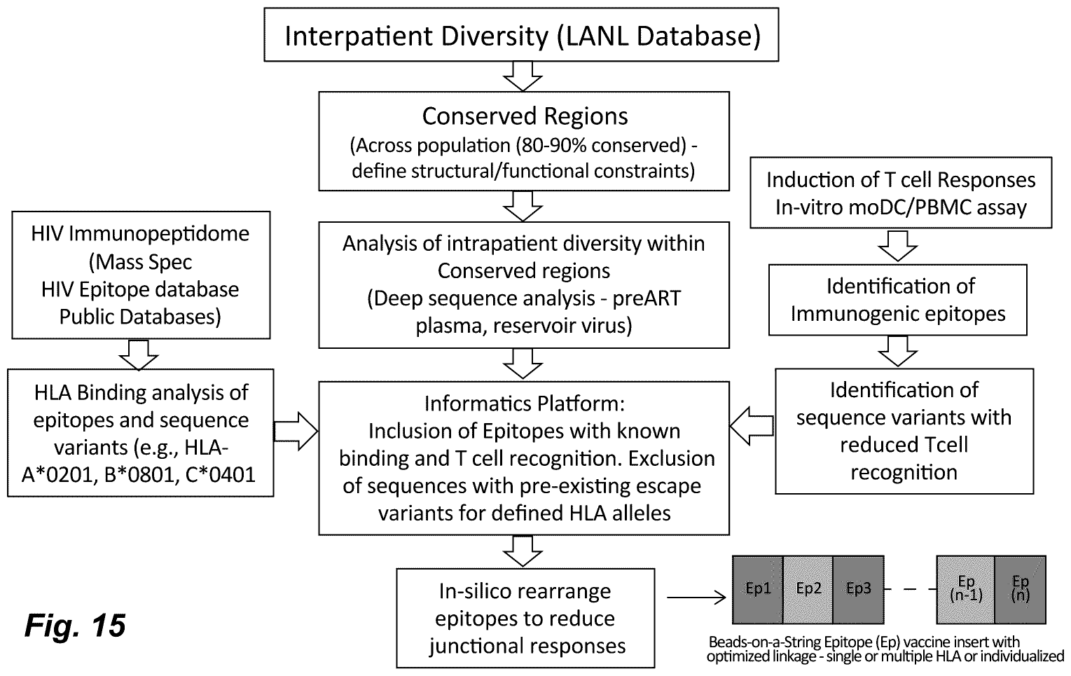

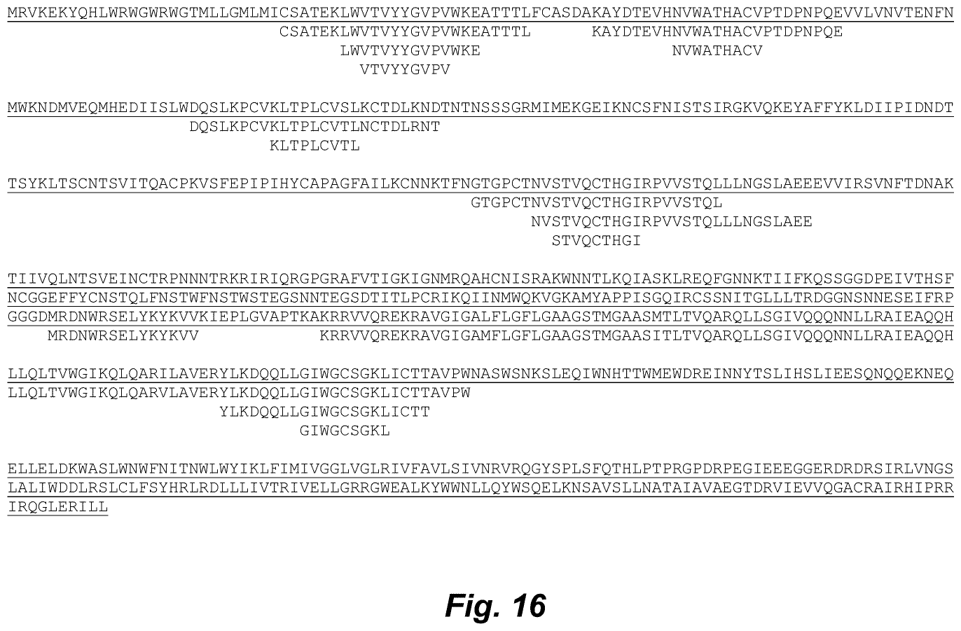

FIG. 14 provides a flow diagram illustrating the basic methodology of the HLA restricted construct (e.g., HLA-A*0201 sequence) approach with deep sequencing data analysis incorporated. SEQ ID NO: 423 provides an exemplary immunogenic fusion polypeptide sequence designed according to this method. It is an illustrative HLA restricted construct improved with deep sequencing data analysis; generated using deep sequencing data from four HLA-A*02:01 patients.

### Example 6

### Viral Expression Vectors Containing Immunogenic Fusion Polypeptides

In this example, we generated viral expression vectors encoding the computationally defined polypeptide segments containing conserved regions of HIV-1 encoded by Gag, Nef and Pol genes as a transgene and confirmed expression of the transgene in mammalian cells. The polypeptide segments containing conserved regions were concatenated or connected by a variety of approaches including direct fusion, linkage of regions by the addition of a proteolytic cleavage site sequence or the addition of a flexible linker between regions. For the purposes of demonstration, we used a polyalanine (AAA) flexible linker (SEQ ID NO: 378), and a proteolytic cleavage site derived from the 2A region of the foot-and-mouth disease virus (FMDV) polyprotein (F2A) (Ryan, et al., J Gen Virol, (1991) 72(11):2727-32).

### Methods

Construction of viral expression vector containing transgene encoding fusion polypeptide variants. Ad5/35 vectors expressing an HIV-1 computationally defined vaccine immunogen with various approaches to linkage of conserved HIV-1 sequences, were generated by in vitro recombination using standard methods (Vector Biolabs). Expression cassettes were generated by PCR using synthetic oligonucleotides codon-biased for improved human expression (GeneArt, ThermoFisher Scientific), and placed under the control of the CMV promoter using standard gene cloning techniques. The constructs developed for this evaluation are listed in Table 1 and schematically depicted in FIG. 20.

TABLE 1
Fusion Polypeptides Expressed from Adenoviral Vectors (“AAA” is SEQ ID NO: 378)

| SEQ | HIV-1 | |
|---|---|---|
| ID | Fusion | |
| NO: | polypeptide | Amino Acid Sequence |
| 349 | p17-p24-Pr- | LKHIVWASRELERFAVNPGLLETVSQNYPIVQNISPRTLNAWVKVVEEKAFSPEVIPMFSALSEGATPQDLNTMLNTVGGH |
| | RT-Int- | QAAMQMLKETINEEAAEWDRLHPVHAGPIAPGQMREPRGSDIAGTTSTLQEQIGWMTNNPPIPVGEIYKRWIILGLNKIVR |
| | RNAseH-nef | MYSPTSILDIRQGPKEPFRDYVDRFYKTLRAEQASQEVKNWMTETLLVQNANPDCKTILKALGPAATLEEMMTACQGVGGP |
| | | GHKARVLAEAMSQLPGRWKPKMIGGIGGFIKVRQYDQGTVLVGPTPVNIIGRNLLTQIGCTLNFPISPIETVPVKLKPGMD |
| | | GPKVKQWPLTEEKIKALVEICTEMEKEGKISKIGPENPYNTPVFAIKKKDSTKWRKLVDFRELNKRTQDFWEVQLGIPHPA |
| | | GLKKKKSVTVLDVGDAYFSVPLDKDFRKYTAFTIPSWGFTTPDKKHQKEPPFLWMGYELHPDKWTVQPIVAKEIVASCDKC |
| | | QLKGEAMHGQVDCSPGIWQLDCTHLEGKIILVAVHVASGYIEAEVIPAETGQETAYFLLKLAGRWPVKTTVKAACWWAGIK |
| | | QEFGIPYNPQSQGVVESMNKELKKIIGQVRDQAEHLKTAVQMAVFIHNFKRKGGIGGYSAGERIVDIIAITKIQNFRVYYR |
| | | DSRDPLWKGPAKLLWKGEGAVVIQDNSDIKVVPRRKAKIIRDYGKQMAGDDCVASRQDEDEEVGFPVKPQVPLRPMTFKGA |
| | | LDLSHFLREKGGLEG |
| 345 | p17-p24- | LKHIVWASRELERFAVNPGLLETAAAVSQNYPIVQNAAAISPRTLNAWVKVVEEKAFSPEVIPMFSALSEGATPQDLNTML |
| | AAA-Pr-RT- | NTVGGHQAAMQMLKETINEEAAEWDRLHPVHAGPIAPGQMREPRGSDIAGTTSTLQEQIGWMTNNPPIPVGEIYKRWIILG |
| | AAA-Int- | LNKIVRMYSPTSILDIRQGPKEPFRDYVDRFYKTLRAEQASQEVKNWMTETLLVQNANPDCKTILKALGPAATLEEMMTAC |
| | RNAseH-AAA- | QGVGGPGHKARVLAEAMSQAAALPGRWKPKMIGGIGGFIKVRQYDQAAAGTVLVGPTPVNIIGRNLLTQIGCTLNFPISPI |
| | nef | ETVPVKLKPGMDGPKVKQWPLTEEKIKALVEICTEMEKEGKISKIGPENPYNTPVFAIKKKDSTKWRKLVDFRELNKRTQD |
| | | FWEVQLGIPHPAGLKKKKSVTVLDVGDAYFSVPLDKDFRKYTAFTIPSAAAWGFTTPDKKHQKEPPFLWMGYELHPDKWTV |
| | | QPIAAAVAKEIVASCDKCQLKGEAMHGQVDCSPGIWQLDCTHLEGKIILVAVHVASGYIEAEVIPAETGQETAYFLLKLAG |
| | | RWPVKTAAATVKAACWWAGIKQEFGIPYNPQSQGVVESMNKELKKIIGQVRDQAEHLKTAVQMAVFIHNFKRKGGIGGYSA |
| | | GERIVDIIAAAAITKIQNFRVYYRDSRDPLWKGPAKLLWKGEGAVVIQDNSDIKVVPRRKAKIIRDYGKQMAGDDCVASRQ |
| | | DEDAAAEEVGFPVKPQVPLRPMTFKGALDLSHFLREKGGLEG |
| 347 | p17-p24- | LKHIVWASRELERFAVNPGLLETVSQNYPIVQNISPRTLNAWVKVVEEKAFSPEVIPMFSALSEGATPQDLNTMLNTVGGH |
| | F2A-Pr-RT- | QAAMQMLKETINEEAAEWDRLHPVHAGPIAPGQMREPRGSDIAGTTSTLQEQIGWMTNNPPIPVGEIYKRWIILGLNKIVR |
| | F2A-Int- | MYSPTSILDIRQGPKEPFRDYVDRFYKTLRAEQASQEVKNWMTETLLVQNANPDCKTILKALGPAATLEEMMTACQGVGGP |
| | RNAseH-F2A- | GHKARVLAEAMSQRAKRAPVKQTLNFDLLKLAGDVESNPGPLPGRWKPKMIGGIGGFIKVRQYDQGTVLVGPTPVNIIGRN |
| | nef | LLTQIGCTLNFPISPIETVPVKLKPGMDGPKVKQWPLTEEKIKALVEICTEMEKEGKISKIGPENPYNTPVFAIKKKDSTK |
| | | WRKLVDFRELNKRTQDFWEVQLGIPHPAGLKKKKSVTVLDVGDAYFSVPLDKDFRKYTAFTIPSWGFTTPDKKHQKEPPFL |
| | | WMGYELHPDKWTVQPIVAKEIVASCDKCQLKGEAMHGQVDCSPGIWQLDCTHLEGKIILVAVHVASGYIEAEVIPAETGQE |
| | | TAYFLLKLAGRWPVKTTVKAACWWAGIKQEFGIPYNPQSQGVVESMNKELKKIIGQVRDQAEHLKTAVQMAVFIHNFKRKG |
| | | GIGGYSAGERIVDIIAITKIQNFRVYYRDSRDPLWKGPAKLLWKGEGAVVIQDNSDIKVVPRRKAKIIRDYGKQMAGDDCV |
| | | ASRQDEDRAKRAPVKQTLNFDLLKLAGDVESNPGPEEVGFPVKPQVPLRPMTFKGALDLSHFLREKGGLEG |
| 428 | p17-p24 | LKHIVWASRELERFAVNPGLLETVSQNYPIVQNISPRTLNAWVKVVEEKAFSPEVIPMFSALSEGATPQDLNTMLNTVGGH |
| | | QAAMQMLKETINEEAAEWDRLHPVHAGPIAPGQMREPRGSDIAGTTSTLQEQIGWMTNNPPIPVGEIYKRWIILGLNKIVR |
| | | MYSPTSILDIRQGPKEPFRDYVDRFYKTLRAEQASQEVKNWMTETLLVQNANPDCKTILKALGPAATLEEMMTACQGVGGP |
| | | GHKARVLAEAMSQ |
| 429 | Pr-RT | LPGRWKPKMIGGIGGFIKVRQYDQGTVLVGPTPVNIIGRNLLTQIGCTLNFPISPIETVPVKLKPGMDGPKVKQWPLTEEK |
| | | IKALVEICTEMEKEGKISKIGPENPYNTPVFAIKKKDSTKWRKLVDFRELNKRTQDFWEVQLGIPHPAGLKKKKSVTVLDV |
| | | GDAYFSVPLDKDFRKYTAFTIPSWGFTTPDKKHQKEPPFLWMGYELHPDKWTVQPI |
| 151 | Nef | EEVGFPVKPQVPLRPMTFKGALDLSHFLREKGGLEG |

Evaluation of target gene expression and F2A cleavage in vitro. To improve assembly of viral vectors encoding the vaccine expression cassette, the genes were cloned into vector plasmids (ThermoFisher Scientific) containing restriction sites for cloning target genes and a GFP marker. DNA was transformed into One Shot™ TOP10 competent cells (Invitrogen, Carlsbad, CA) following manufacturer's protocol and plated onto LB agar plate supplemented with 100 μg/ml ampicillin. The plate was incubated overnight at 37° C. A single colony was picked from the plate and inoculated into a 10 ml liquid LB+ampicillin culture and shaken overnight at 37° C. at 250 rpm in an Eppendorf bench top shaker. The bacterial pellet was processed using QIAprep Spin miniprep kit (Qiagen, Germantown, MD) to obtain the plasmid DNA following manufacturer's protocol. Nucleic acid concentration was determined by reading absorbance at 280 nm using NanoDrop™ 2000 (Thermo Scientific). To evaluate in vitro expression, the expression vectors were transfected into Expi293™ cells according to manufacturer's protocol using ExpiFectamine™ (Invitrogen, Carlsbad, CA). At Day2 post-transfection when the viability of cells was still at >80%, they were evaluated for GFP expression by flow cytometry or pelleted. The cell lysates were evaluated for HIV-1 gag p24 expression by ELISA or protein expression was determined by western blot immunoprecipitated with anti-Nef antibody to enable detection of the full-length translation product containing the Nef sequence at the C terminus.

### Results

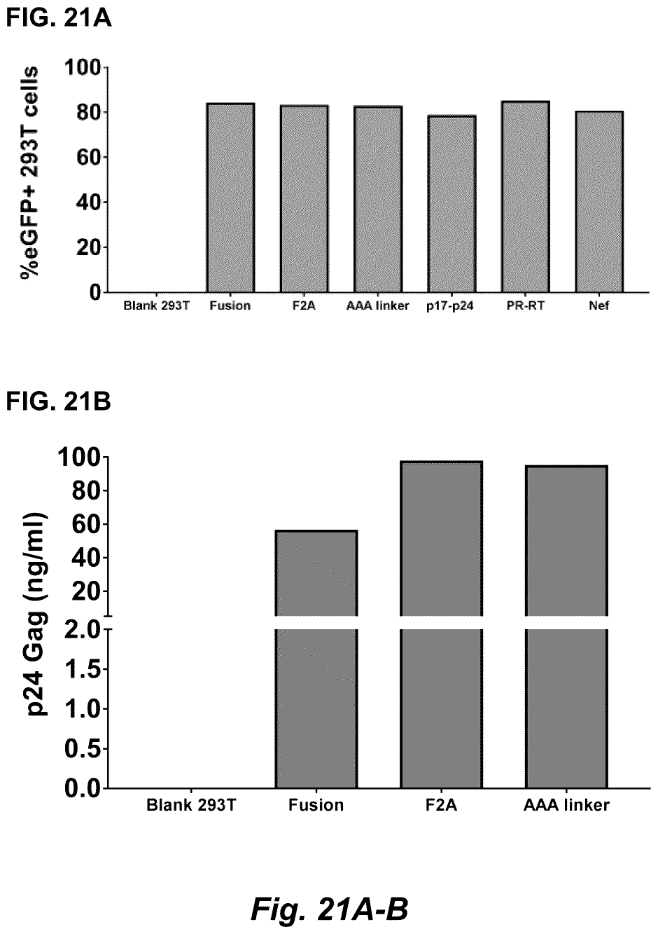

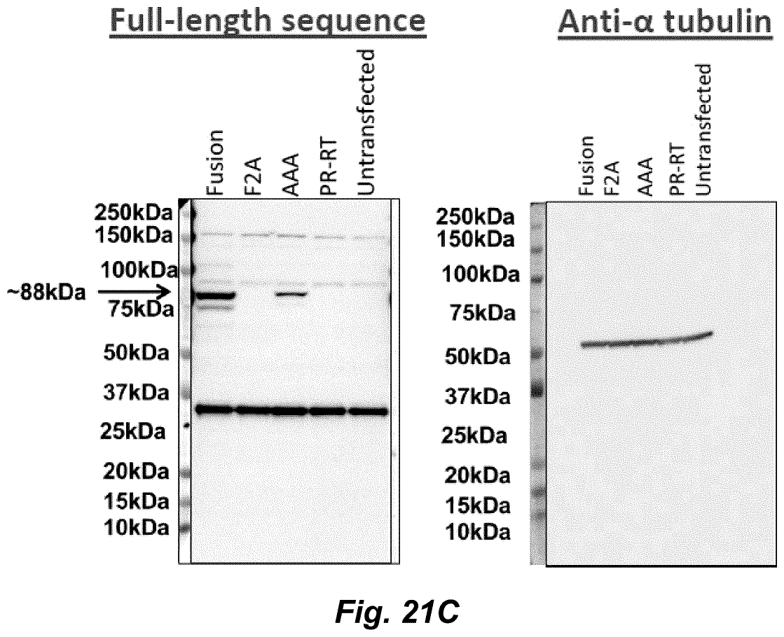

The data depicted in FIGS. 21A-21C demonstrated that the all three approaches to concatenation or connection of conserved region polypeptide segments into fusion polypeptides resulted in the efficient transfection and expression of the polypeptides encoded by the transgene. Evaluation of the translation product indicated that the inclusion of the F2A proteolytic cleavage sequence resulted in appropriate cleavage of the polypeptide (FIG. 21C). We then tested the efficiency of these constructs in various viral vector systems to prime T cell responses in vitro and in vivo.

### Example 7

### In Vitro Assays Demonstrating Human T Cell Activation Induced by Fusion Polypeptides

In this example, we established an in vitro method for testing the efficacy of T cell priming in humans by vaccine constructs in expression vectors. A similar approach is described in, e.g., WO 2015/110397. The application of this method in vaccinology allows evaluation of antigen processing, presentation and priming of T cells in humans of the transgene cassette, as well as the study of immune parameters including adjuvants and immune modulators that may modify the efficacy of priming.

### Methods

Monocyte purification and maturation of monocyte derived dendritic cells (moDCs). Freshly isolated or cryopreserved PBMCs were used in the moDC-based T cell stimulation assays. CD14+ monocytes were purified from PBMCS from individuals with or without HIV, and ART naïve or on ART using the EasySep human anti-CD14 positive selection antibody kit (StemCell Technologies). Flow cytometry was used to confirm the purification of the isolated CD14+ monocytes to >90% prior to the establishment of the culture. To generate immature moDCs, 2×10⁶ purified CD14+ monocytes were cultured in 3 mL of moDC differentiation media, i.e., complete RPMI 1640 containing 10% heat inactivated fetal calf serum, 1% penicillin streptomycin/mL, 0.5 mM HEPES, 800U/mL of GM-CSF (Miltenyi Biotec), and 1000U of IL-4 (Miltenyi Biotec) in 6 well culture plates. The plates were incubated at 37° C. and 5% CO₂ for 6 days and monitored daily to ensure adherence of monocytes. To generate mature moDCs, adherent immature moDC cultures were supplemented with recombinant soluble CD40L (0.5 μg/ml), IFN-γ (1,000U/ml), PGE2 (5 μM), TNF-α (10 ng/ml), IL-6 (100 ng/ml) and IL-1β (10 ng/ml) with an additional 3 ml of moDC differentiation media on day 6 and incubated at 37° C. and 5% CO₂ for an additional 48 hrs.

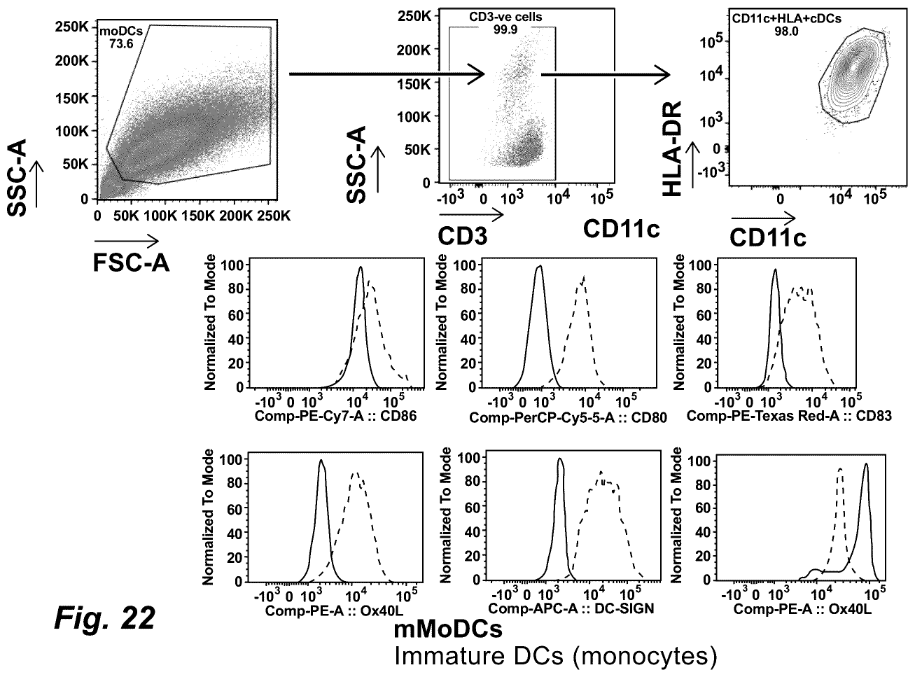

On day 8, adherent mature moDCs were detached using ice-cold PBS and a cell scrapper to manually detach the moDCs. Following this procedure, unattached cells were washed using moDC differentiation media and transferred to a 50 ml Falcon tube. The resulting cell mixture was centrifuged at 1500 rpm for 5 minutes at room temperature. Next, the supernatant was discarded and the cell pellet was resuspended in 5 ml of moDC differentiation media. A fraction of the mature moDCs were isolated and stained to characterize the differentiation phenotype of the moDCs with antiCD11c+, anti-HLA-DR+, anti-CD14−, anti-CD40+, anti-DCSIGN+, anti-CD83, anti-CD86 and anti-OX40L antibodies. The results are shown in FIG. 22.

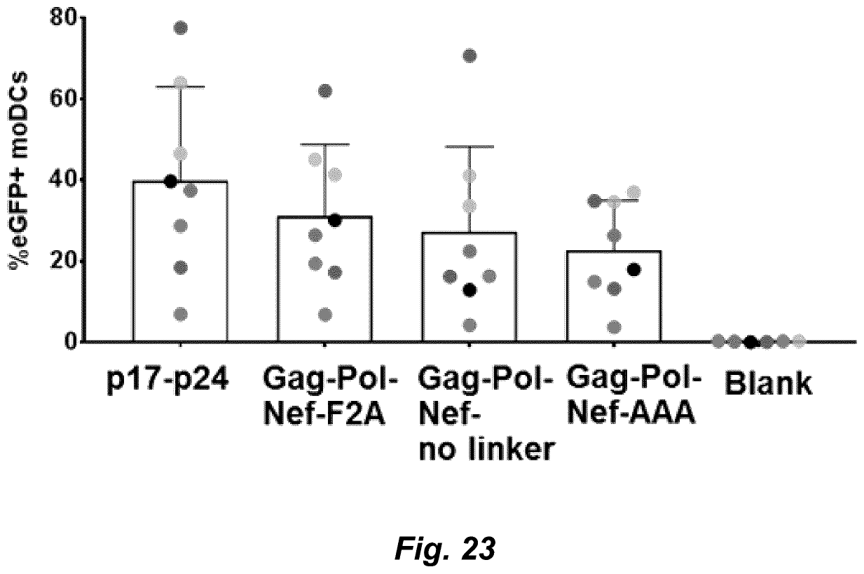

Transduction of moDCs with viral vector, e.g., adenovirus (Ad) 5/35 vectors. The purified moDCs were harvested, washed twice in serum-free media, and re-suspended in X-Vivo 15 (BioWhittaker, Walkersville, MD) at 10⁷/ml. Cells were equilibrated at 37° C. in a water bath for 20-30 min before transduction. Ad5/35 stocks were thawed on ice and added to the moDC suspension at the indicated multiplicity of infection (MOI). Cells were gently mixed and placed immediately in the 37° C. incubator. After 2 hours, warm moDC differentiation media containing GM-CSF and IL-4 were added to dilute the moDCs to a final concentration of 10⁵/ml. 0.5 ml of transduced moDCs were transferred to 48 well plates and maintained at 37° C. in 5% CO₂ for an additional 24 h before addition of PBMCs or purified CD8+ T cells. The results are shown in FIG. 23.

Purification of autologous CD8 or CD4 T cells and co-culture with moDCs. In experiments requiring the stimulation of CD8+ T cells, the CD8+ fraction was enriched from autologous PBMCs using the human CD8+ T cell enrichment kits (EasySep, StemCell Technologies). Purified CD8+ T cells were co-cultured with vaccine vector transduced autologous moDCs for 7 days (first round) and then non-adherent cells were subsequently transferred to a second culture of freshly transduced autologous moDCs for another 7 days (day 14 second round). In experiments requiring the stimulation of CD4+ T cells, the CD4+ fraction was enriched from autologous PBMCs using the human CD4+ T cell enrichment kits (EasySep, StemCell Technologies). Cells were confirmed to have >90% purity by flow cytometry. Isolated cells were resuspended at 1-5×10⁶/ml cells in 1.0 ml (max. volume) of PBS containing 0.1% FBS in a 15 ml Falcon tube and labeled with cell trace violet (Tag-it violet) following the manufacturer's protocol (Biolegend). Cell-trace violet (CTV)-labeled CD8+ T cells, CD4+ T cells or PBMCs were then enumerated and resuspended at 2×10⁶/ml. 1×10⁶ purified CD8+ T cells, CD4+ T cells or PBMCs were then seeded to each well containing 5×10⁴ moDCs in 48 well culture plates at a moDC: T cell/PBMC ratio of 1:20.

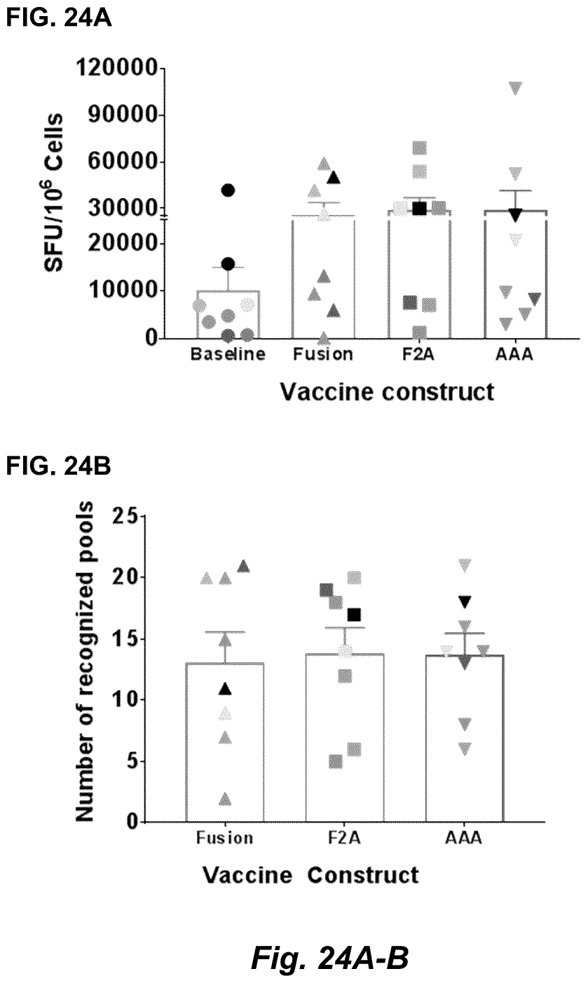

ELISpot Assays. Pre-coated strip ELISpot plates (Cellular Technologies Limited) were used for all ELISpot analyses. Briefly, 5×10⁴ cells from Day 10 moDC-CD8+ T cell/PBMC cultures were seeded to each well. Peptide pools consisting of 15-mer peptides overlapping by 11 amino acids spanning the entire HIV conserved regions were assembled into a matrix with 8-12 aa in each pool and used in IFN-γ ELISpot assays to evaluate vaccine immunogenicity. For positive controls, 50 ng/ml PMA (Sigma) was added. Plates were incubated at 37° C. in 5% CO₂ for 24 hours. After 24 hours stimulation, the cells were removed from the plates and the wells were washed three times in PBS prior to three washes with PBS containing 0.05% tween. Biotinylated anti-IFN-γ detection antibody was then added to the plates for 2 hours at room temperature. The plates were then washed three times with PBS containing 0.05% tween prior to the addition of streptavidin-conjugated alkaline phosphatase (AP). Wells were then washed two times with 0.05% tween-PBS and then two times with distilled water prior to the addition of the blue developer solution. The plates were then incubated at room temperature for 15 minutes before the reaction was stopped using tap water. The wells were then dried overnight and spot forming units (SFUs) were counted on an Immunospot ELISpot reader. The settings were identical for all plates and counts were expressed at SFU per 10⁶ PBMCs. The results are shown in FIGS. 24A-B.

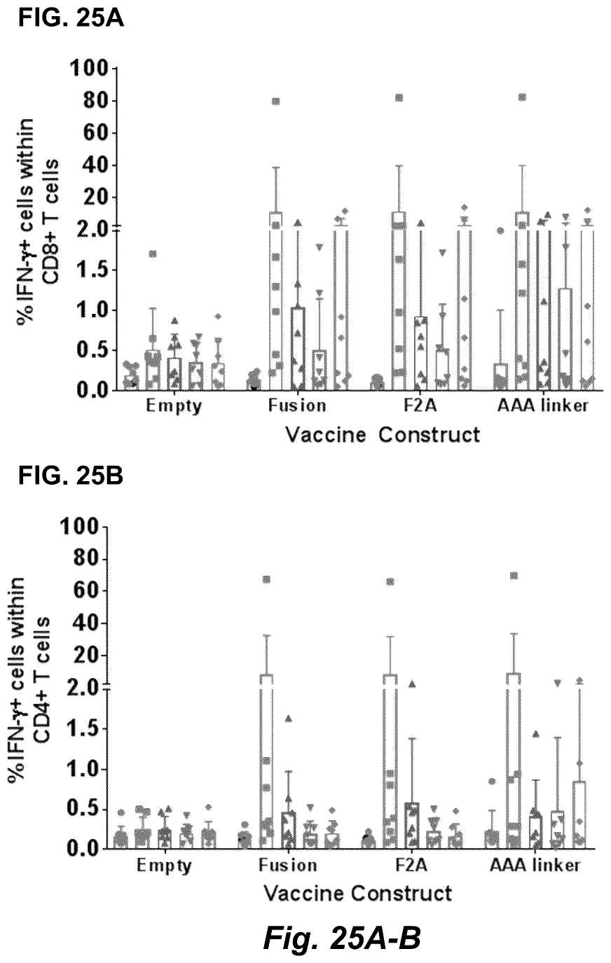

In vitro Peptide Stimulation and Intracellular Cytokine Staining. Lymphocytes were incubated at 37° C. for 5 h with 2 μg/ml of the corresponding HIV peptide pools along with anti-CD107a, and GolgiPlug (BD) (1 μl/ml) and monensin (1×) were added during the last 4 h of re-stimulation. This was followed by surface and intracellular staining for cytokine production. The Foxp3 Fixation/Permeabilization Concentrate and Diluent Kit (Thermo Fisher Scientific) was used for intracellular cytokine staining. Briefly, after blocking Fc receptors with 0.5 mg/ml human IgG (BD), 1×10⁶ cells were incubated with a mixture of fluorescence-conjugated anti-human antibodies for 30 min at 4° C. Stained cells were washed twice using FACS buffer (PBS, 2% FCS, 0.1% NaN₃), acquired with an LSR II flow cytometer using FACSDiva software (BD), and analyzed using FlowJo software version 10.2 (TreeStar). Anti-human antibodies were obtained from BioLegend or BD biosciences, anti-PD-1 BV421 or BV605 clone EH12.2H7, anti-CD27 BV711 clone 0323, anti-CD4 BV605 clone OKT4, anti-CCR7 BV785 clone G043H7, anti-CD45RA PE-Cy7 clone H100), anti-CD3 BV650 clone SK7, anti-CD8a BV650 clone RPA-T8. After surface staining, stained cells were incubated with 100 μl of Fix/Perm b 667 buffer for 1 hour. Subsequently, cells were washed twice with 100 μl Perm 77jhy gt buffer each time and were then incubated with a cocktail of antibodies diluted in 100 μl of Perm buffer per 1×10⁶ cells. A cocktail of fluorophore-conjugated anti human antibodies containing anti-IL-2 PE clone MQ1-17H12, anti-TNF-α PercPcy5.5 clone MAB11 and anti-IFN-γ PE-CF594 clone B27, was added to the cells and stained for an hour. For mouse experiments, anti-IFN-γ PE clone XMG1.2, anti-IL-2 APC-cy7 clone JES6-5H4 and anti-TNF-α BV650 clone MP6-XT22. Permeabilized cells were then washed with 100 μl Perm buffer twice and immediately analyzed on a Fortessa flow cytometer. Results are shown in FIGS. 25A-B.

### Results

Monocyte derived DCs matured in the presence of cytokines (GM-CSF, IL-4, CD40L, IFN-γ, PGE2, TNF-α, IL-6 and IL-1β) and transduced with viral vectors containing a vaccine transgene were able to prime autologous vaccine antigen specific T cells in vitro. These responses were of high magnitude and breadth. Patient to patient variability is observed in transduction efficiency of moDCs and may reflect variability in expression of receptors to facilitate uptake of viral vectors as would be anticipated in a heterogeneous human population. The assay may facilitate the preclinical evaluation of vaccine constructs across large numbers of human donors prior to initiation of large-scale vaccine trials.

Human donors who respond to priming with generation of de novo responses made responses of similar magnitude irrespective of the fusion approach used to combine the conserved regions. Donors with high magnitude responses consistently recognized a larger number of pools irrespective of the viral vector construct used to prime T cell responses.

The method primed both CD4+ and CD8+ T cell responses. The immunodominant CD8+ responses were targeted at p24 Gag and Nef while immunodominant CD4+ responses were mostly focused on p24 Gag. There was no significant difference in the magnitude of the response or the regions targeted based on the approach used to combine conserved regions in the design of the vaccine construct. The primed T cells were largely monofunctional with IFN-γ being the primary cytokine with comparable proportions of multifunctional T cell responses across several donors. This likely reflects the viral vectors used (e.g., adenovirus) as well as the in vitro culture techniques.

The results demonstrate strong induction of CD8+ and CD4+ T cell responses by vaccine immunogen in primary human PBMCs. Fusion of conserved regions results in responses of similar magnitude to responses induced by an immunogen with an F2A proteolytic cleavage sequence or an AAA linker (SEQ ID NO: 378). This data supports the use of any of these approaches to fusion of conserved regions. Factors such as packaging limits of a vaccine vector, or reducing or avoiding the generation of junctional responses, are considerations that contribute determining which approach is used for a given set of sequences or viral vector.

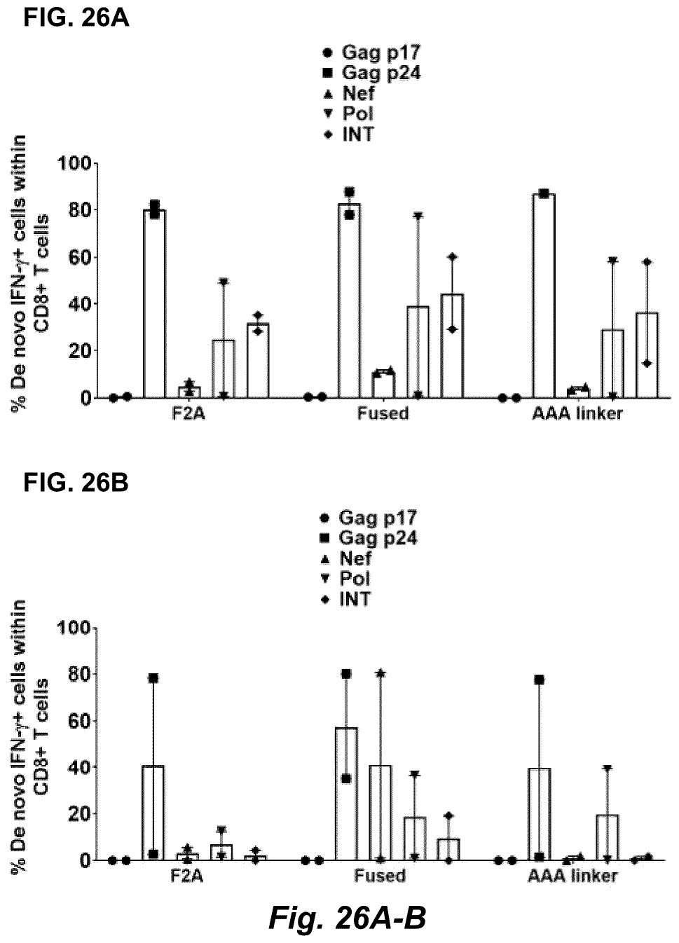

The data are consistent with the conclusion that CD8+ T cells from aviremic and viremic patients that were exposed to two rounds of moDC stimulation had an enhanced magnitude of IFN-γ regardless of the concatenation approach used (FIG. 26A-26B).

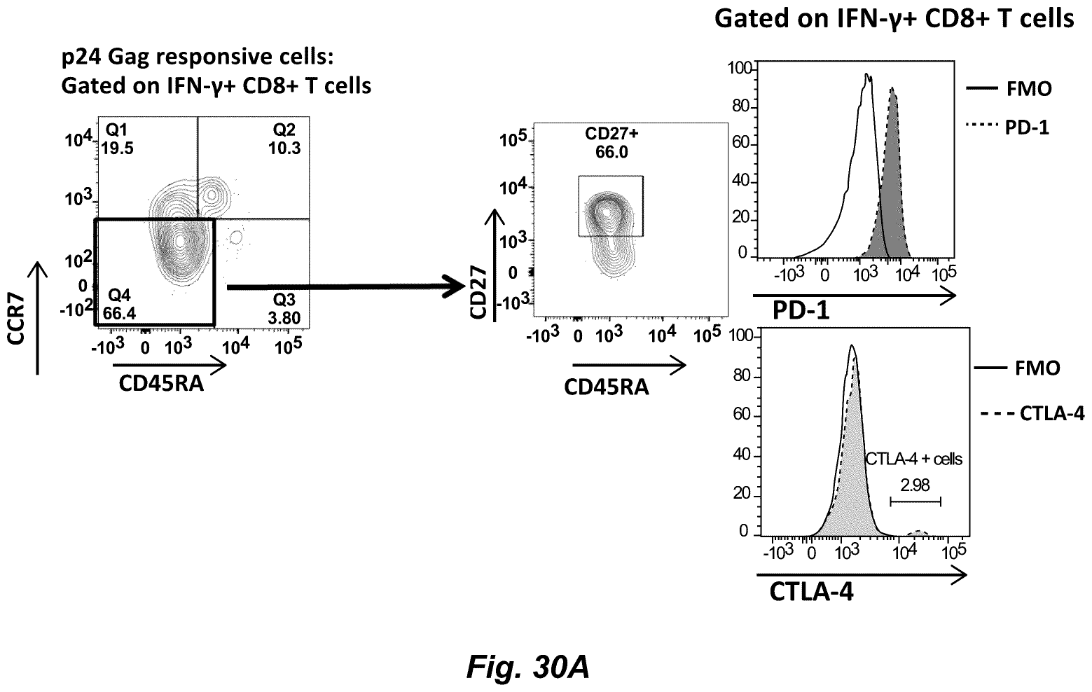

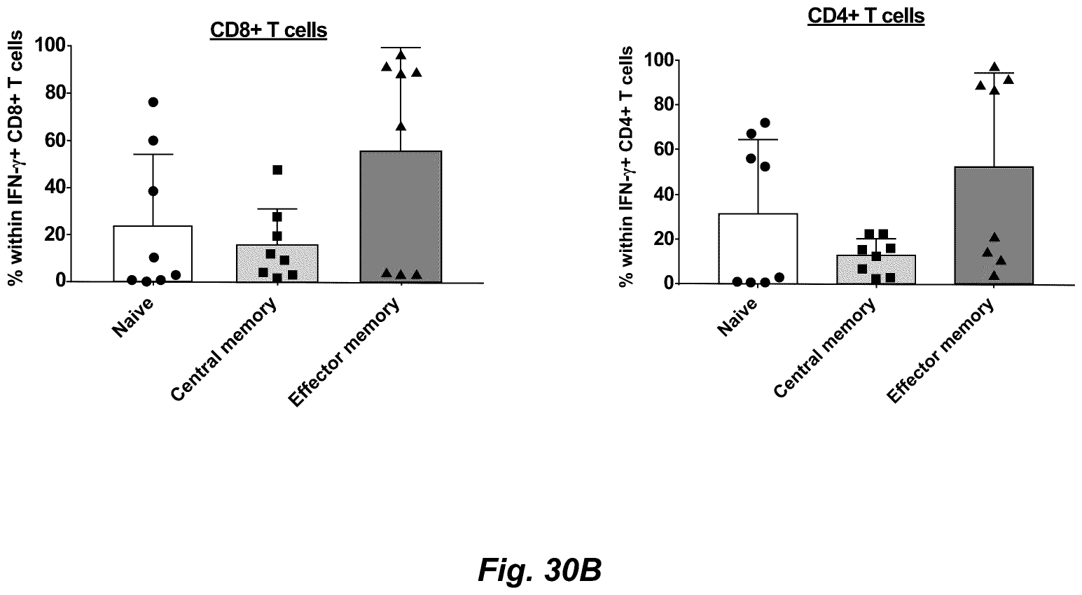

The results further demonstrate that the in vitro moDC-T cell priming assay induce both de-novo naïve responses as well as prime pre-existing memory responses and that IFN-γ producing cells express high levels of PD-1 and CTLA-4, suggesting that the responding cells were exhausted as illustrated in FIGS. 30A-30B.

### Example 8

### In Vivo T Cell Activation Assays

In this example, we evaluated the efficacy of in vivo T cell priming by vaccine constructs in a mouse model and determined the optimal approach to fusing conserved regions within the vaccine construct. To do this, we immunized groups of mice with Ad5/35 vectors expressing computationally defined conserved regions vaccine immunogen sequences with different linkage strategies. We evaluated the magnitude and functional phenotype of those responses to determine the optimal approach to fusion of conserved regions.

### Methods

### In-Vivo Evaluation of Immunogenicity

Immunizations. Six or seven-week-old C57BL/6 and Balb/c mice were immunized with either 1×10⁸ or 1×10⁹ PFU of Ad5/35 vectors expressing HIV immunogens by intramuscular (i.m.) injections in both hind leg muscles. The vaccine vector was administered in 100 μl of phosphate-buffered saline (PBS) injections (50 μl per quadriceps). Mice were anesthetized with isoflurane prior to vaccine immunization. Animals were housed at the Charles River Laboratories animal facility (North Carolina) and experiments were performed according to approved IACUC protocol.

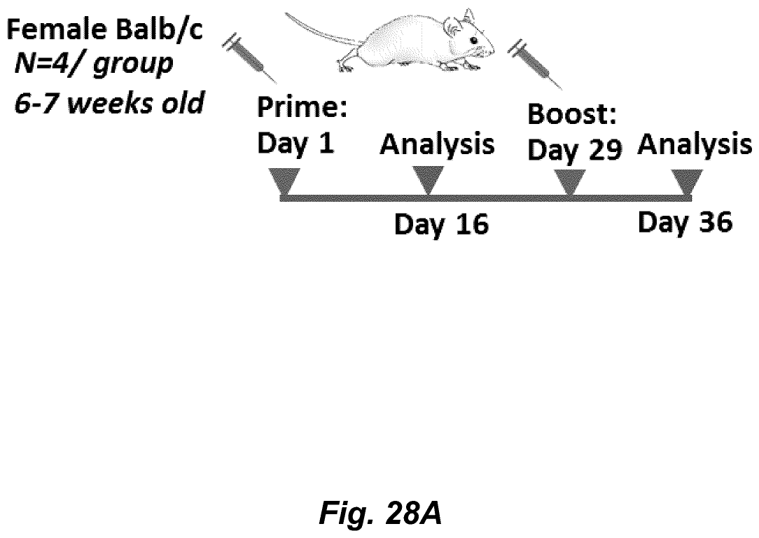

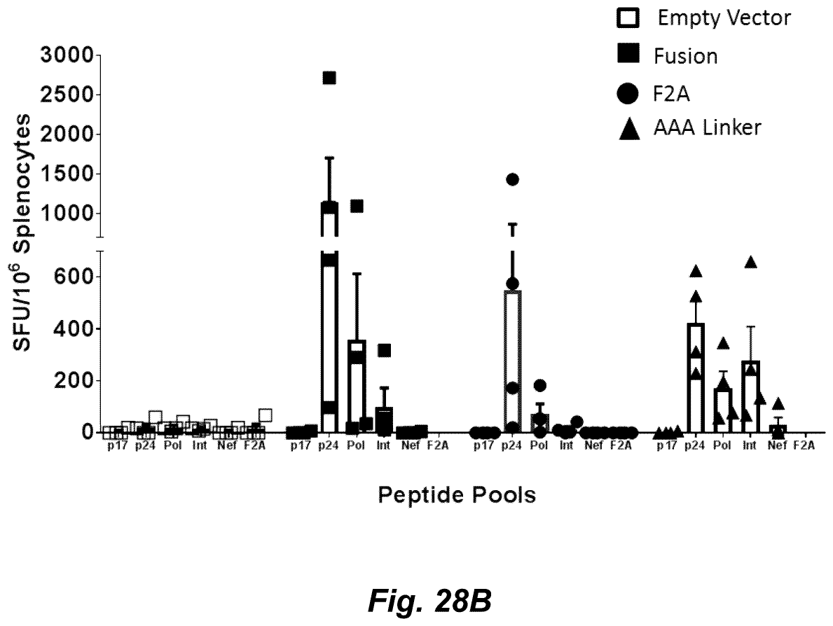

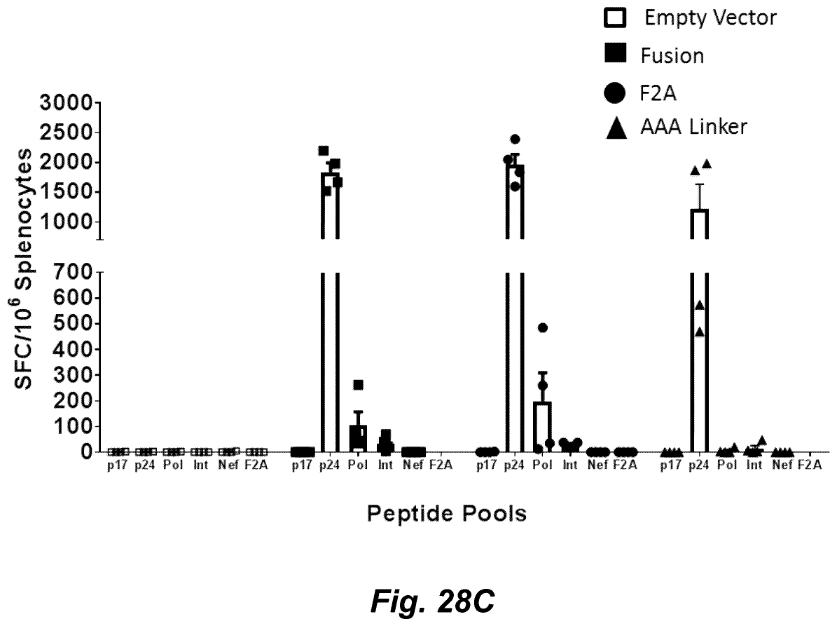

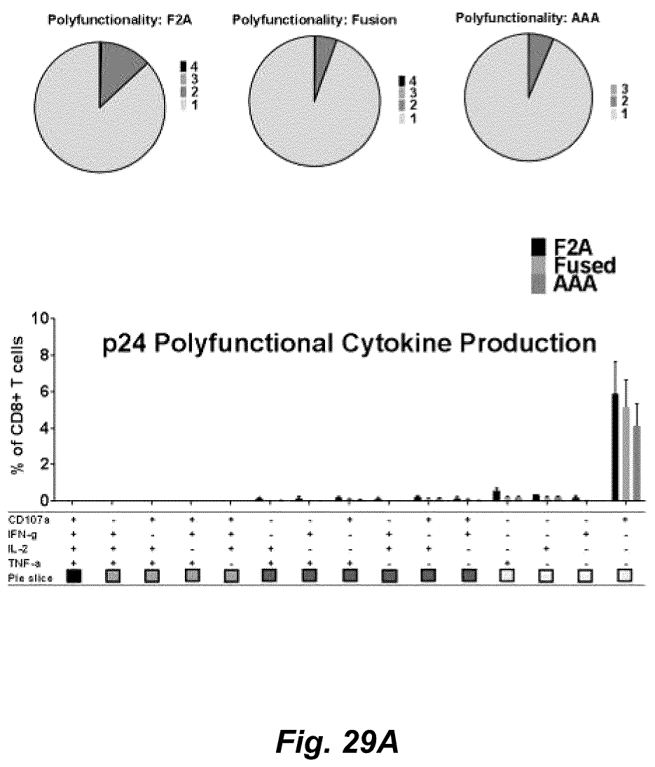

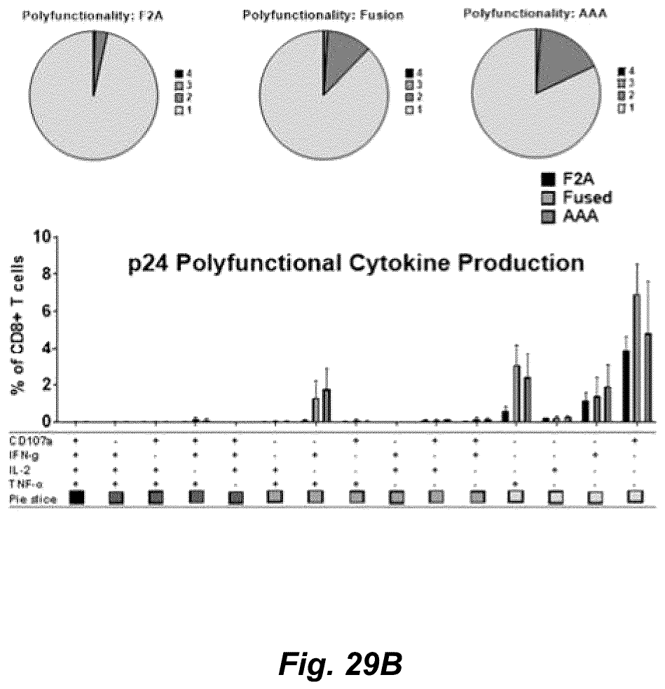

Homologous prime-boost regimen. Mice were primed with either 1×10⁸ or 1×10⁹ PFU of Ad5/35 vectors expressing HIV immunogens by intramuscular (i.m.) injections in both hind leg muscles and rested for 28 days before homologous boost with vectors expressing identical antigens. Immunogenicity and cellular phenotype were evaluated by analyzing splenocytes by ELISpot assay as previously described (Miyahira, et al., J Immunol Methods, (1995) 181(1):45-54), ICS or tetramer staining at various time points. A schematic of the regimen and results are shown in FIGS. 28A-28C.

Flow cytometry. Cell counts for prepared single-cell suspensions were determined using a hemacytometer. After blocking Fc receptors with 1:100 anti-CD16+CD32 (Biolegend) (for mouse cells) or 0.5 mg/ml human IgG (BD) (for human cells), 1×10⁶ cells from single-cell suspensions were incubated with a mixture of fluorescence-conjugated anti-mouse or anti-human antibodies for 30 min at 4° C. Stained cells were washed twice using FACS buffer (PBS, 2% FCS, 0.1% NaN₃), acquired with an LSR II flow cytometer using FACSDiva software (BD), and analyzed using FlowJo software version 10.2 (TreeStar). Anti-mouse antibodies were obtained from either Biolegend or BD Biosciences, CD8 AF700 clone 53-6.7, CD4 BV605 clone RM4-5, TCR-β PECF594 clone H57-597, CD27 BV711 clone LG.3A10, CD43 PE-cy7 clone 1B11, KLRG1 PercpCy5.5 clone 2F1 and CD127 BV421 clone SB/199 were used for surface staining. After surface staining, cells were fixed and permeabilized in preparation for intracellular cytokine staining. The Foxp3 Fixation/Permeabilization Concentrate and Diluent Kit (Thermo Fisher Scientific) were used for intracellular cytokine staining. Briefly, 1×10⁶ cells already stained with surface antibodies were incubated with 100 μl of Fix/Perm buffer for 1 hour. Subsequently, cells were washed twice with 100 μl Perm buffer each time and were then incubated with a cocktail of antibodies diluted in 100 μl of Perm buffer per 1×10⁶ cells. A cocktail of fluorophore-conjugated anti mouse anti-IFN-γ PE clone XMG1.2, anti-IL-2 APC-cy7 clone JES6-5H4 and anti-TNF-α BV650 clone MP6-XT22 were used for intracellular cytokine staining. Permeabilized cells were then washed with 100 μl Perm buffer twice and immediately analyzed on a Fortessa flow cytometer.

Anti-mouse antibodies were obtained from either Biolegend or BD Biosciences, CD8 AF700 clone 53-6.7, CD4 BV605 clone RM4-5, TCR-β PECF594 clone H57-597, CD27 BV711 clone LG.3A10, CD43 PE-cy7 clone 1B11, KLRG1 PercpCy5.5 clone 2F1 and CD127 BV421 clone SB/199 were used for surface staining. After surface staining, cells were fixed and permeabilized in preparation for intracellular cytokine staining. The Foxp3 Fixation/Permeabilization Concentrate and Diluent Kit (Thermo Fisher Scientific) were used for intracellular cytokine staining. Briefly, 1×10⁶ cells already stained with surface antibodies were incubated with 100 μl of Fix/Perm buffer for 1 hour. Subsequently, cells were washed twice with 100 μl Perm buffer each time and were then incubated with a cocktail of antibodies diluted in 100 μl of Perm buffer per 1×10⁶ cells. A cocktail of fluorophore-conjugated anti mouse anti-IFN-γ PE clone XMG1.2, anti-IL-2 APC-cy7 clone JES6-5H4 and anti-TNF-α BV650 clone MP6-XT22 were used for intracellular cytokine staining. Permeabilized cells were then washed with 100 μl Perm buffer twice and immediately analyzed on a Fortessa flow cytometer.

### Results

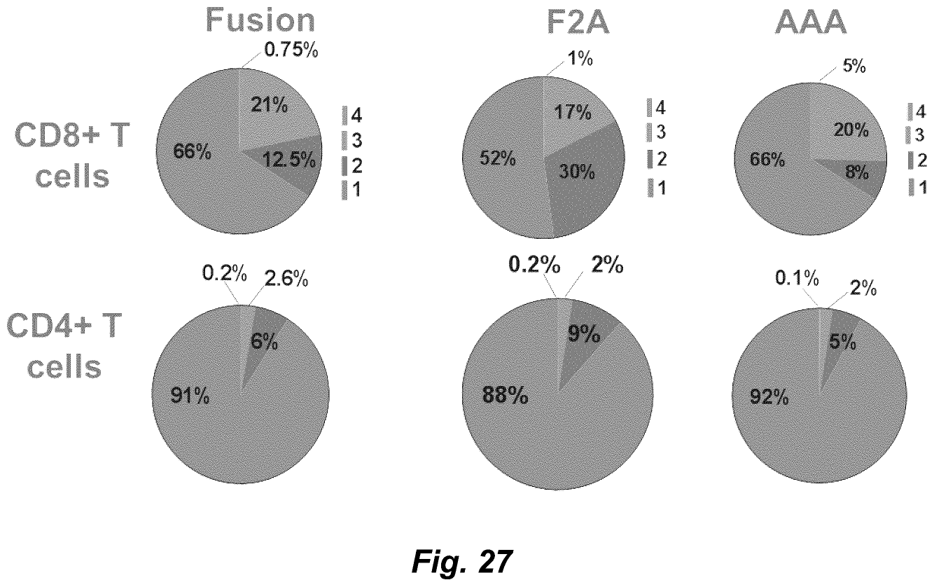

The viral vectors expressing conserved regions of HIV-1 proteins in fusion polypeptides were able to elicit high magnitude responses following prime and boost when expressed in Ad5/35 vectors. No responses were generated to the F2A sequence. The magnitude of the response was peptide pool specific. The Gag p24 response was of the greatest magnitude with weaker responses observed to Pol (PR/RT) and Int, and almost no Nef specific responses observed (FIG. 27B). This reflects the presence of immunodominant epitopes presented by mouse HLA within Gag p24 (Im, et al., PLoS Pathog, (2011) 7(5):e1002041). The magnitude of the p24 response was greatest in the mice immunized with the fusion and F2A constructs. Levels of responses changed following homologous boost, with the response to p24 emerging as the dominant response (FIG. 28C). Responses were of similar magnitude in animals vaccinated with all constructs. No significant different was observed in the magnitude of the response in mice vaccinated with a vector where the concatenation of conserved regions was by fusion, F2A or AAA linker (SEQ ID NO: 378). Similar studies were conducted with C57/Bl6 mice. The overall magnitude of the IFN-γ response was weaker, however following both prime and boost there was no significant difference in the magnitude of the response induced by the different vaccine constructs.

The ability to produce cytokines is a functional measure of effector and memory CD8+ T cells. We evaluated the phenotypic and functional characteristics of CD8+ T cell responses generated following immunization. We observed that following Ad5/35 immunization, T cells with monofunctional characteristics were generated. The dominant monofunctional response identified was the presence of CD107a expression, among the single cytokines IFN-γ, was the most commonly produced. There were not functional differences observed across the groups based on the fusion approach used to design the vector transgene.

The data are consistent with the conclusion that the conserved regions sequences are immunogenic. There was no significant difference in in vivo mouse immunogenicity based on the transgene sequence insert approach to fusing conserved regions.

### Example 9

### Evaluating the Ability of Leader Sequences to Enhance Immunogenicity of Fusion Polypeptides

In this example, our primary objective was to determine if leader or signal sequences could enhance the immunogenicity of the HIV-1 vaccine immunogen. To do this, we designed vaccine constructs with various signal sequences and expressed them in viral vectors, e.g., adenovirus or arenavirus vectors.

Viral vectors can be engineered to express protein sequences that can enhance antigen processing or presentation in both MHC class I and class II pathways. These leader sequences are well known. These sequences are typically short polypeptides with hydrophobic domains, that bind to signal recognition particles and direct the elongating protein to membrane bound structures, e.g., endoplasmic reticulum or lysosomes. These secretory signal sequences may include a secretory polypeptide from tissue plasminogen activator (tPA), GM-CSF signal peptides (SPCore) or from chemokines such as the monocyte chemoattractant protein 3 (MCP3) or C-X-C motif chemokine ligand 10 (CXCL10; a.k.a., IP-10). These are often placed at the N-terminus of the vaccine immunogen expression cassettes. Other signaling sequences may include N-terminal and C-terminal sequences from the lysosomal associated membrane protein 1 or 2 (LAMP-1 or -2) which directs proteins to the lysosomal compartment. The secretory polypeptide from secreted chemokines such as MCP-3 and IP-10 can be fused and engineered onto the N-terminus of the vaccine immunogen. The addition of destabilizing sequences that can promote ubiquination and consequently target a sequence for degradation have been previously described and used in the context of immunization with HIV or SIV immunogens (Tobery, et al., J Exp Med, (1997) 185(5):909-20; Townsend, et al., J Exp Med, (1988) 168(4):1211-24). An N-terminal β-catenin signal sequence can promote N-terminal ubiquitination promoting degradation by the 26S proteasome (Rosati, et al., Proc Natl Acad Sci USA, (2009) 106(37):15831-6).

Transgenic mice that express human HLA molecules represent a unique in vivo experimental model for evaluating human immune system function. These models have been used to study the role of the human class I or class II restricted T cell repertoire in autoimmune disease, infectious disease, and vaccine development. These mice serve as tools to evaluate vaccine design strategies through epitope identification and to study T cell responses restricted by HLA molecules which can facilitate the development of vaccines targeted against specific ethnic/regional populations, as well as broad populations. The HLA transgenic mouse models generally assume conserved antigen processing and presentation of antigen to generate the appropriate ‘human’ 8-10 mer CTL epitopes, physiological peptide selection by human class 1 molecules in murine antigen presentation cells, and an appropriate CD8+ T-cell repertoire containing T-cell receptors (TCRs) capable of positive selection on the ‘human’ epitope-HLA class I complex.

We determined whether the signal sequences could increase the CD8+ and CD4+ T cell responses following immunization of C57/BL6, Balb/c or A*0201 transgenic mice with adenovirus or arenavirus vectors expressing the HIV immunogen with the corresponding leader/signal sequences. We evaluated the phenotype, magnitude and functional characteristics of those responses.

Construction of viral vectors expressing vaccine transgene with various leader sequences. Adenovirus (Ad5/35 or Ad5) or arenavirus vectors expressing computationally defined HIV conserved regions sequences (e.g., SEQ ID NOs: 353-356, 363-366 and 358-372) with various leader sequences were generated. Expression plasmids and viral vectors were synthesized as previously described. Illustrative leader sequences used in the test constructs are summarized in the table below.

TABLE 2
Signal Sequences Expressed with Immunogenic
Fusion Polypeptides

| SEQ | | |
|---|---|---|
| ID | Source | |
| NO: | Protein | Signal Sequence |
| 393 | CSF2, GM-CSF | MWLQSLLLLGTVACSISV |
| 394 | PLAT, t-PA | MDAMKRGLCCVLLLCGAVFVSAR |
| 398 | CCL7, MCP-3 | MNPSAAVIFCLILLGLSGTQGILDMAQPVGIN |
| | | TSTTCCYRFINKKIPKQRLESYRRTTSSHCPR |
| | | EAVIFKTKLDKEICADPTQKWVQDFMKHLDKK |
| | | TQTPKLASAGA |
| 397 | β-catenin | MRKAAVSHWQQQSYLDSGIHSGATTTAPSLS |
| 399 | lysosomal | MAPRSARRPLLLLLLLLLLGLMHCASAAMFMV |
| | associated | KNGNGTACIMANFSAAFSVNYDTKSGPKNMTL |
| | membrane | DLPSDATVVLNRSSCGKENTSDPSLVIAFGRG |
| | protein 1 | HTLTLNFTRNATRYSVQLMSFVYNLSDTHLFP |
| | (LAMP-1) | NASSKEIKTVESITDIRADIDKKYRCVSGTQV |
| | (N-terminus) | HMNNVTVTLHDATIQAYLSNSSFSRGETRCEQ |
| | | DRPSPTTAPPAPPSPSPSPVPKSPSVDKYNVS |
| | | GTNGTCLLASMGLQLNLTYERKDNTTVTRLLN |
| | | INPNKTSASGSCGAHLVTLELHSEGTTVLLFQ |
| | | FGMNASSSRFFLQGIQLNTlLPDARDPAFKAA |
| | | NGSLRALQATVGNSYKCNAEEHVRVTKAFSVN |
| | | IFKVWVQAFKVEGGQFGSVEECLLDENSLEDI |
| 412 | LAMP-1 - | GSEFTLIPIAVGGALAGLVIVLIAYLVGRKRS |
| | C-term | HAGYQTI |

### Methods

### In-Vivo Evaluation of Immunogenicity

Immunizations. Six or seven-week-old Balb/c mice were immunized with 1×10⁹ PFU of Ad5/35 vectors by intramuscular (i.m.) injections in both hind leg muscles or 1×10⁶ RCV FFU for replication defective LCMV vectors expressing HIV immunogens by intravenous (i.v.) injections. The Ad5/35 vaccine vectors were administered in 100 μl of phosphate-buffered saline (PBS) injections (50 μl per quadriceps). The LCMV vaccine vectors were administered in a volume of 200 μl formulated in buffer (10 mM Hepes, 150 mM NaCl, 20 mM Glycine, pH 7.4 (±0.2). For stabilization 10% Sorbitol was added. Mice were anesthetized with isoflurane prior to vaccine immunization. Animals were housed at the Charles River Laboratories animal facility (North Carolina) and experiments were performed according to approved IACUC protocol.

ELISpot Assays. Pre-coated strip ELISpot plates (Cellular Technologies Limited) were used for all ELISpot analyses. Briefly, 2×10⁵ splenocytes from immunized animals were seeded to each well. Peptide pools consisting of 15-mer peptides overlapping by 11 amino acids spanning the entire HIV or A*0201 conserved regions sequences were used in IFN-γ ELISpot assays to evaluate vaccine immunogenicity. For positive controls, 50 ng/ml PMA (Sigma) was added. Plates were incubated at 37° C. in 5% CO₂ for 24 hours. After 24 hours stimulation, the cells were removed from the plates and the wells were washed three times in PBS prior to three washes with PBS containing 0.05% tween. Biotinylated anti-IFN-γ detection antibody was then added to the plates for 2 hours at room temperature. The plates were then washed three times with PBS containing 0.05% tween prior to the addition of streptavidin-conjugated alkaline phosphatase (AP). Wells were then washed two times with 0.05% tween-PBS and then two times with distilled water prior to the addition of the blue developer solution. The plates were then incubated at room temperature for 15 minutes before the reaction was stopped using tap water. The wells were then dried overnight and spot forming cells (SFCs) were counted on an Immunospot ELISpot reader. The settings were identical for all plates and counts were expressed at SFU per 10⁶ splenocytes.

### Results

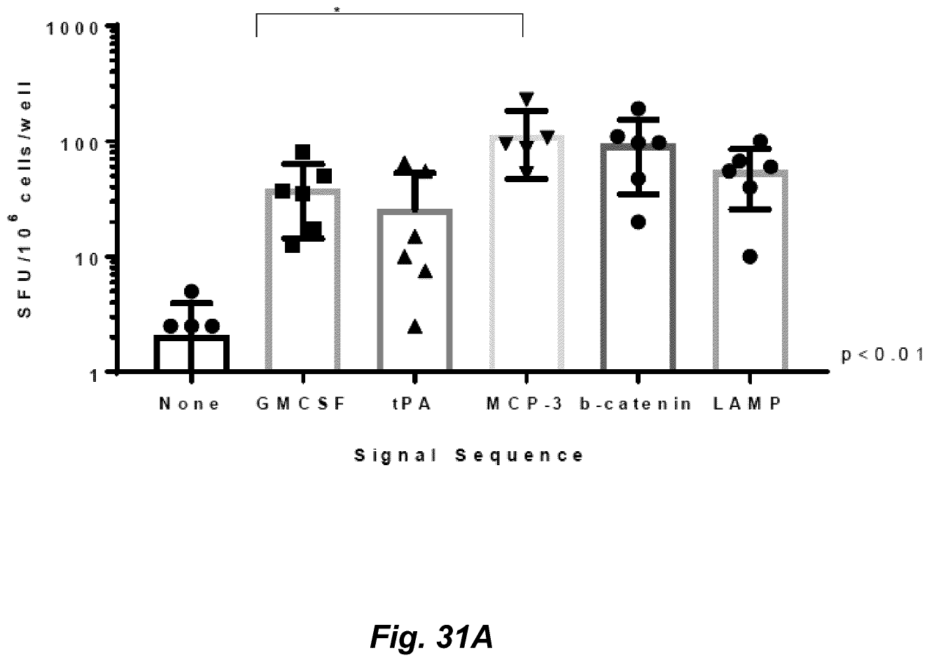

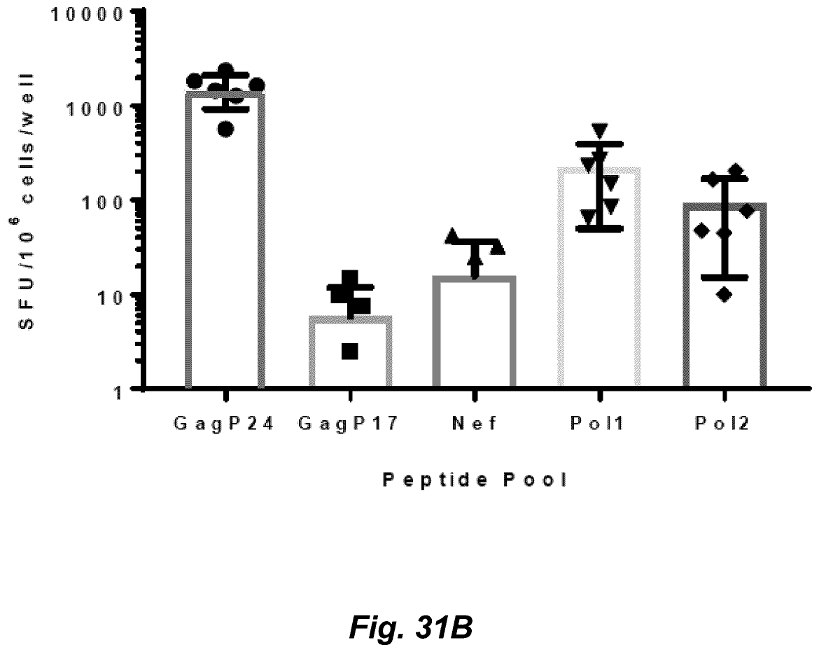

Leader sequences enhanced immunogenicity of the vaccine immunogen in an Ad5/35 vector in A*0201 transgenic mice immunized with a vaccine construct consisting of HIV-1 sequences designed by our algorithm and predicted to bind to HLA-A*0201. The addition of the signal sequences GM-CSF, tPA, MCP-3, β-catenin and LAMP significantly enhanced immunogenicity of the constructs relative to the construct without the leader sequences. In the model with A*0201 transgenic mice MCP-3 was significantly more immunogenic than vectors expressing GM-CSF (p<0.01). See, FIG. 31A.

In addition the full-length conserved regions construct with a GM-CSF leader sequence was (FIG. 31B) was immunogenic in A*0201 mice, with immunodominant responses observed within HIV-1 Gag p24.

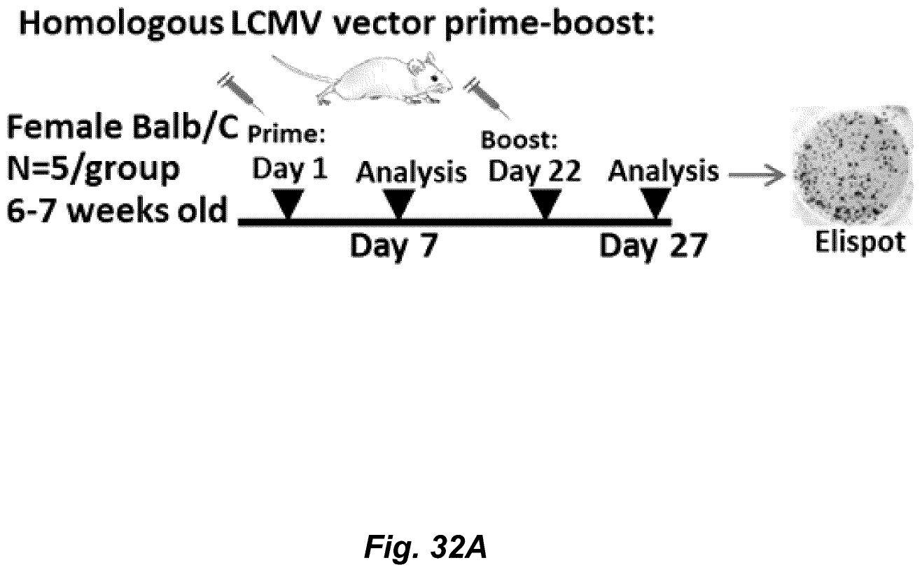

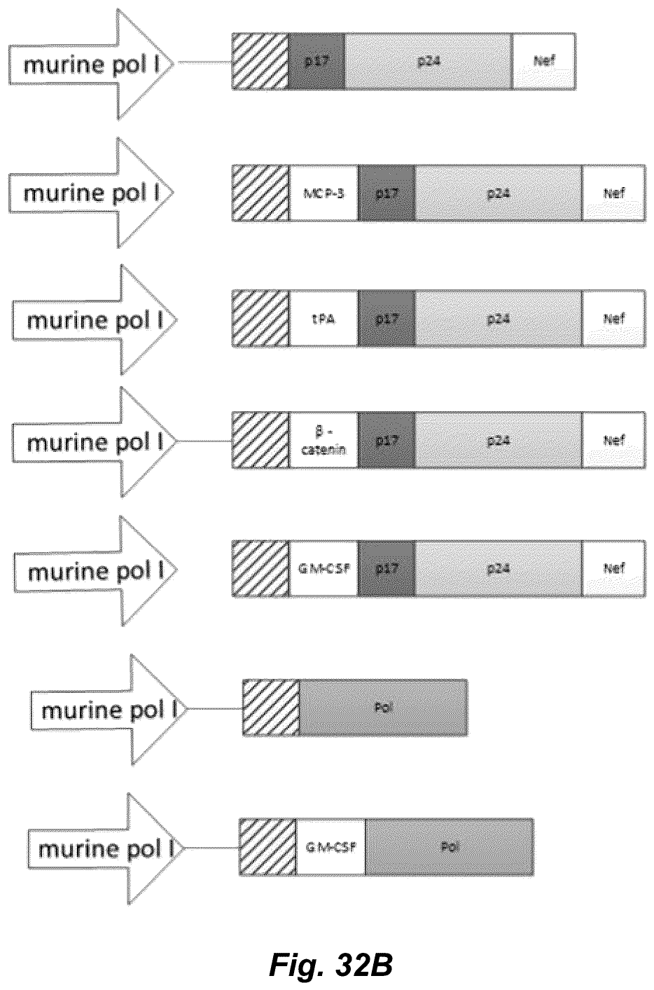

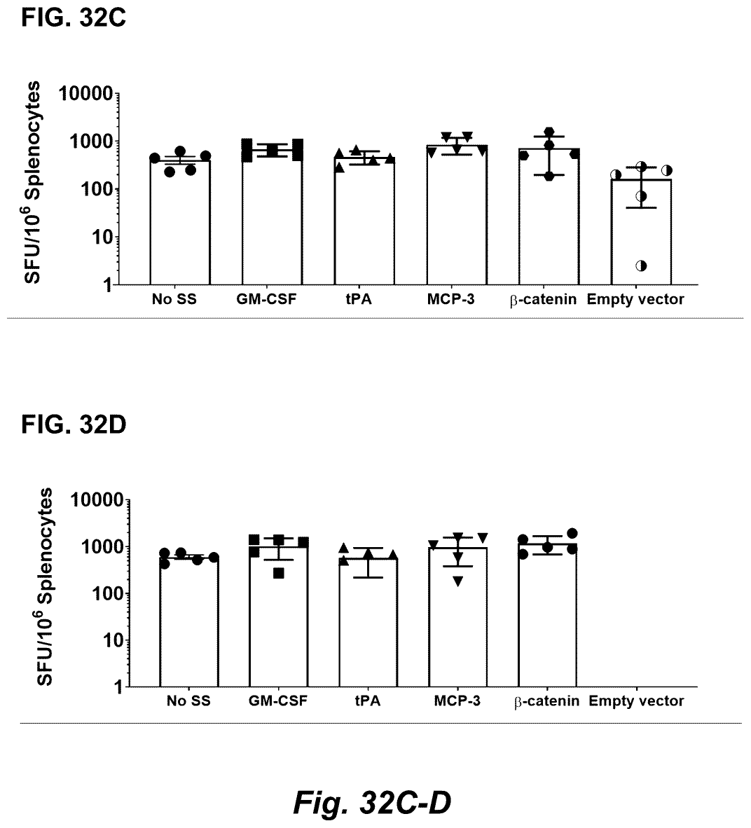

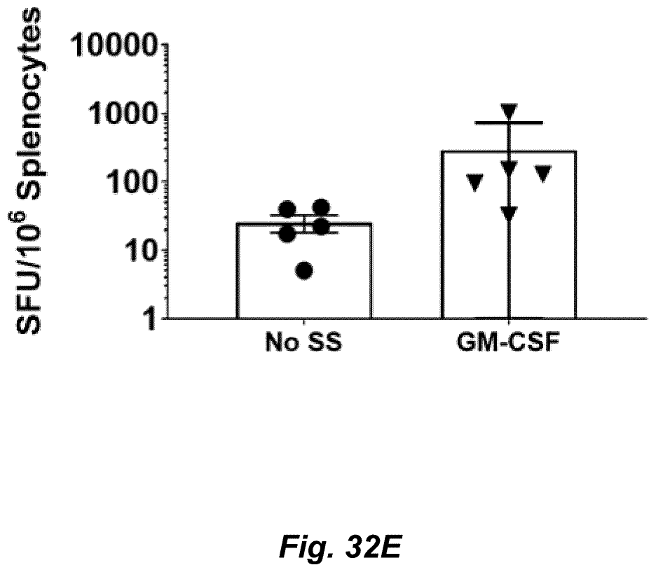

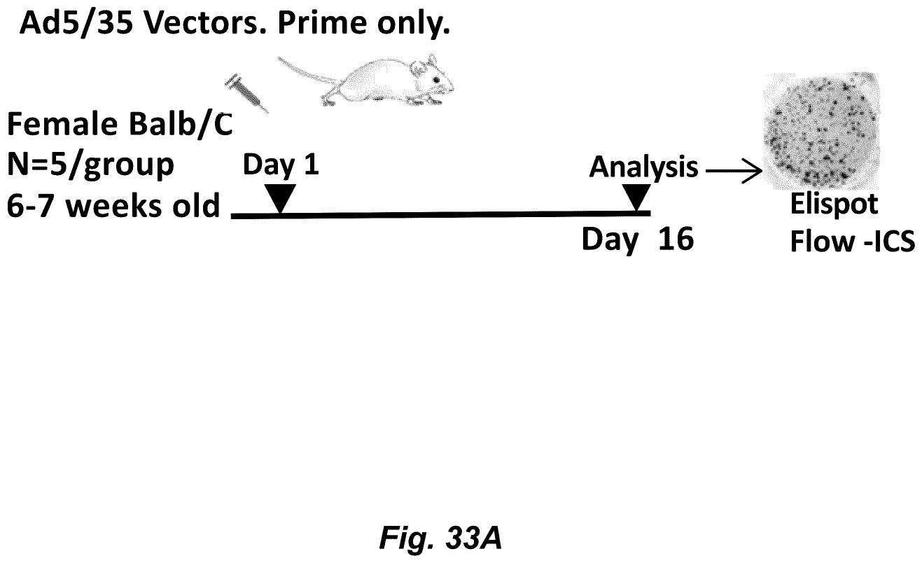

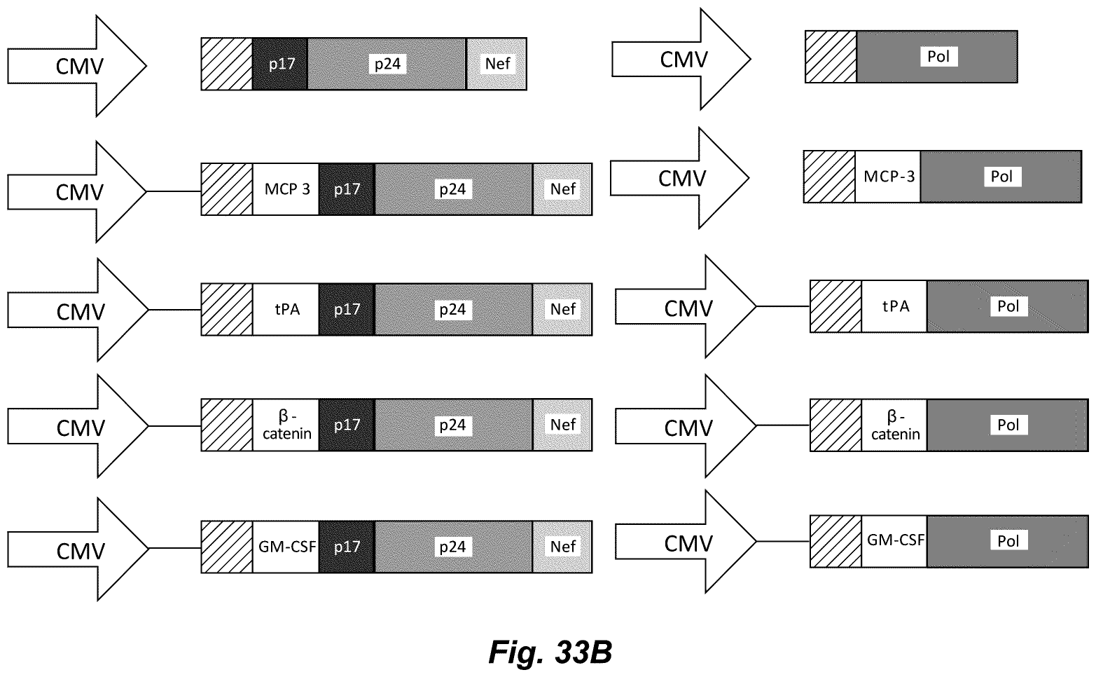

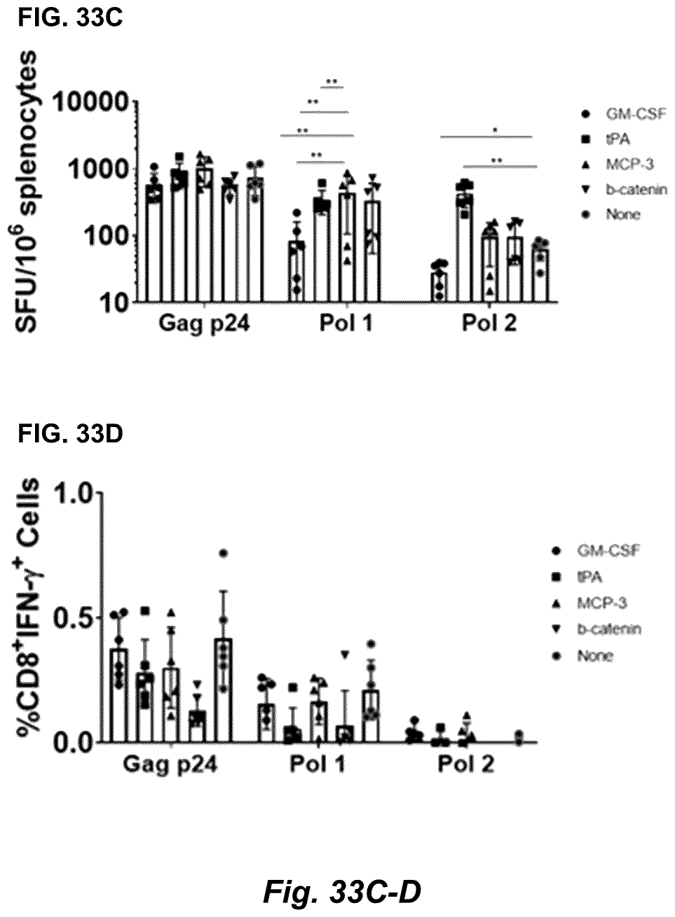

The data are consistent with the conclusion that signal sequences can enhance the immunogenicity of Gag p24 epitopes with LCMV replication incompetent vectors following prime and boost immunizations. The data are further consistent with the conclusion that GM-CSF signal sequence enhances the immunogenicity of subdominant antigens such as Pol (FIGS. 32C-32E).

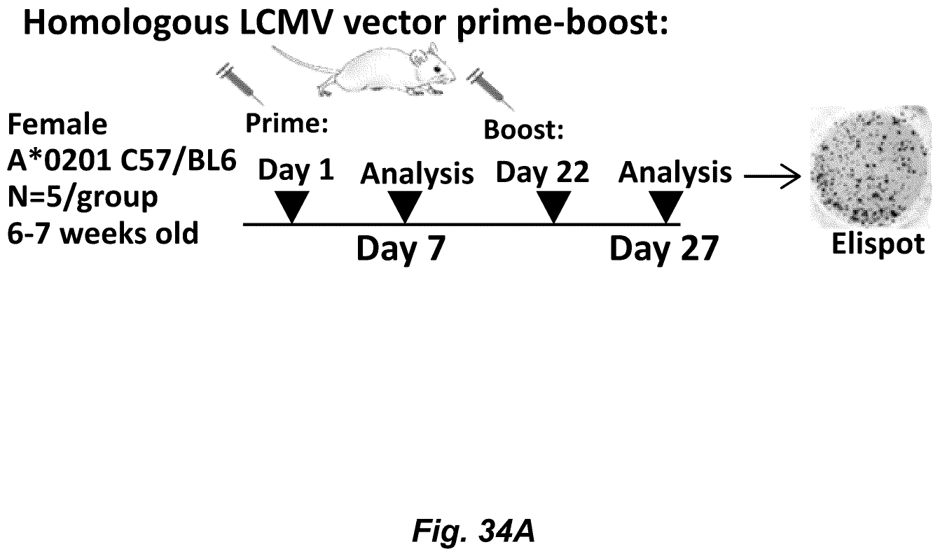

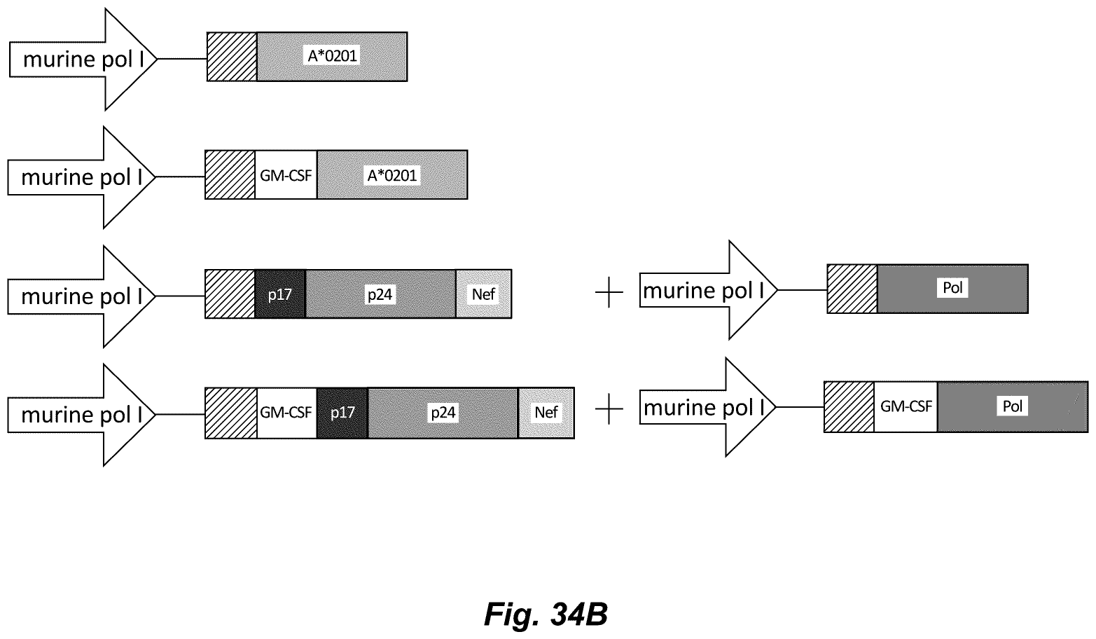

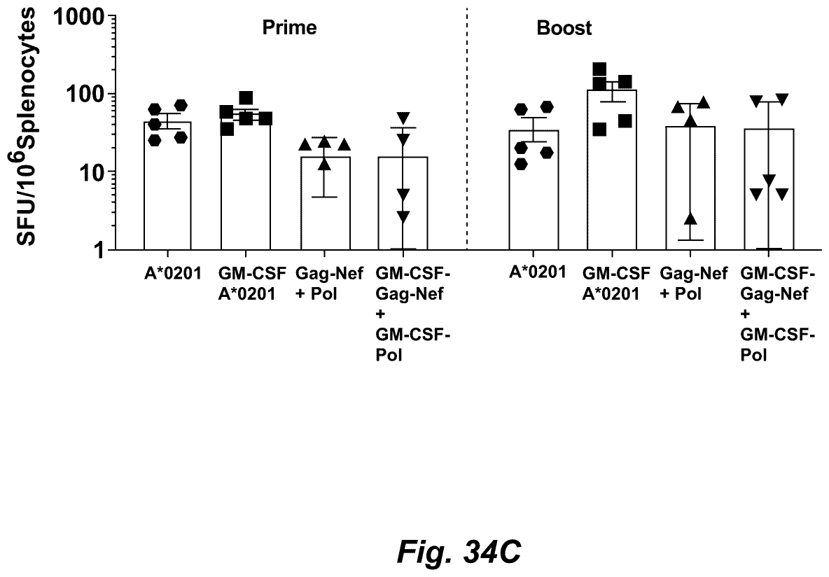

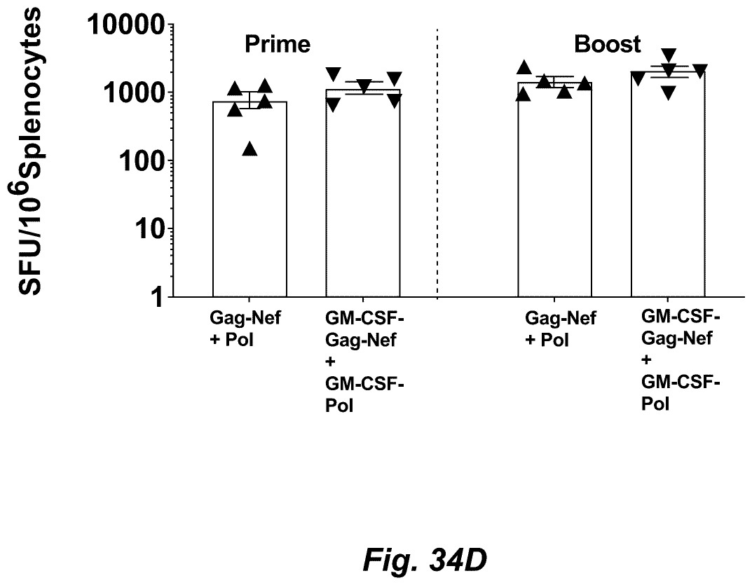

C57/Bl6 mice transgenic for human leucocyte antigen (HLA) A*0201 antigen-binding domain were used to evaluate immunogenicity of vaccines composed of defined HLA A*0201-restricted cytotoxic T-lymphocyte (CTL) epitopes from HIV conserved region sequences in LCMV replication incompetent vector format with and without GM-CSF leader sequence. Magnitude of IFN-γ responses were evaluated at day 7 after prime and day 5 after boost vaccinations. The data are consistent with the conclusion that immunogenicity of A*0201 epitopes from HIV conserved sequences in a “bead on a string” format is weak. Upon homologous boost, responses with GM-CSF leader sequence were enhanced slightly but were not significant. Gag specific responses in mice vaccinated with HIV conserved sequence showed enhanced responses compared to A*0201 peptide specific responses. Boost and GM-CSF leader sequences further enhanced responses from prime and no leader sequence responses but were not significantly different. The data indicates that the responses in A*0201 transgenic mice are likely driven by C57/BL6 background and the endogenic processing and presentation of A*0201 specific epitopes on HLA A*0201 allele is not optimal. The data are further consistent with the conclusion that signal sequences can enhance the immunogenicity of A*0201 and Gag p24 epitopes with LCMV replication incompetent vectors following prime and boost immunizations (FIGS. 34A-34D).

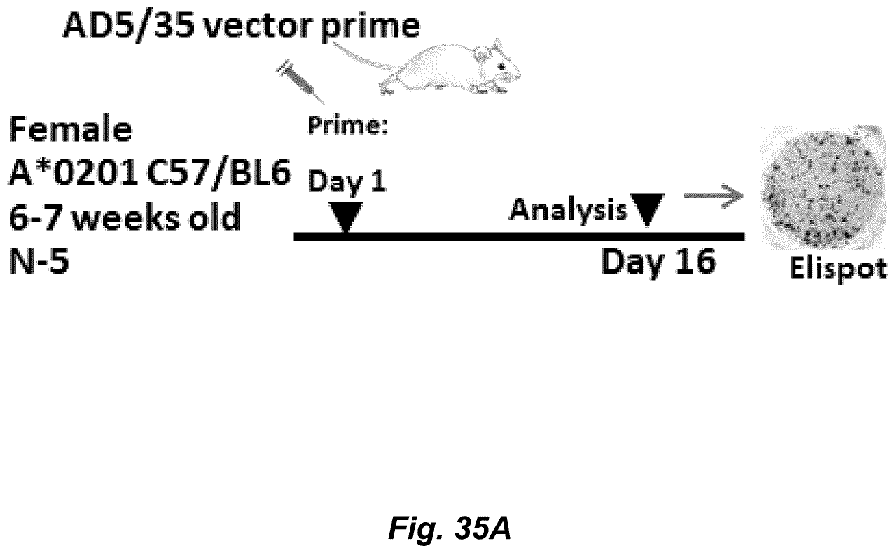

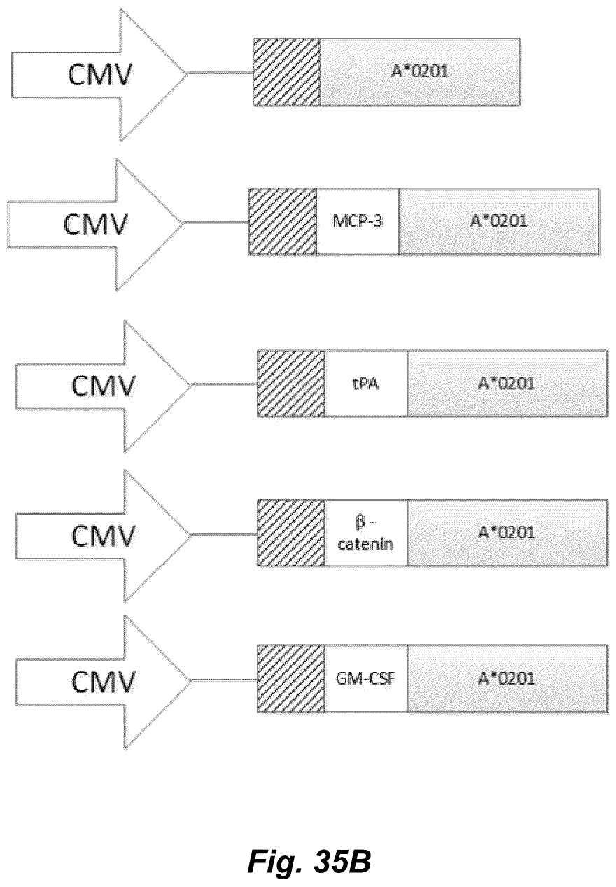

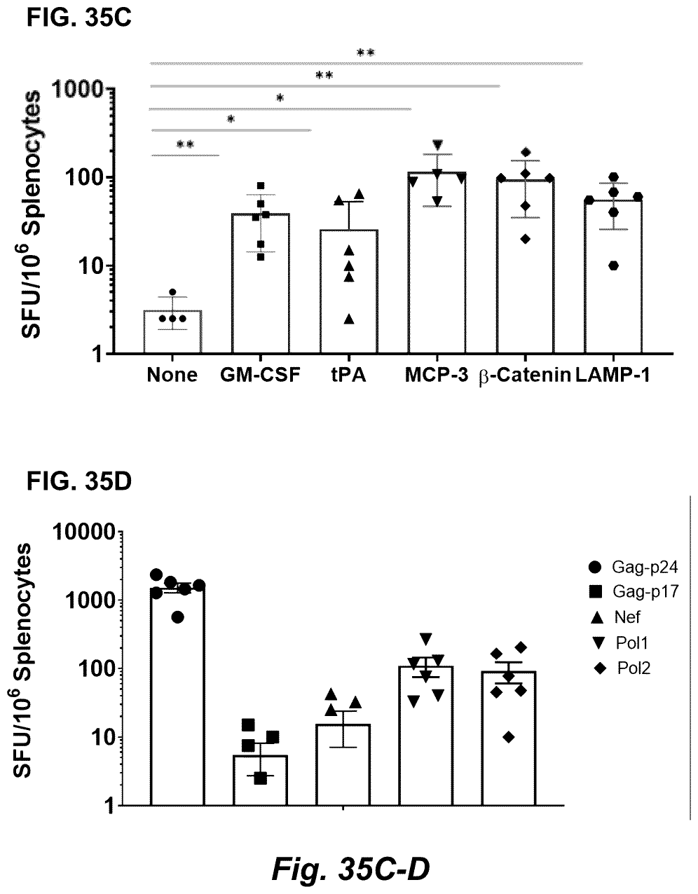

C57/Bl6 mice transgenic for human leucocyte antigen (HLA) A*0201 antigen-binding domain were used to evaluate immunogenicity of vaccines composed of defined HLA A*0201-restricted cytotoxic T-lymphocyte (CTL) epitopes from HIV conserved region sequences in Ad5/35 vector format with and without various leader sequences. Magnitude of IFN-γ responses were evaluated at day 16 after prime vaccination. The data are consistent with the conclusion that immunogenicity of A*0201 epitopes from HIV conserved sequences in a “bead on a string” format is weak. In the presence of leader sequences including, GM-CSF, TPA, MCP-3, β-Catenin and LAMP-1, the responses were enhanced significantly, even though the magnitude of response is smaller. The data are consistent with the conclusion that signal sequences can enhance the immunogenicity of A*0201 epitopes with Ad5/35 vectors following prime immunizations. Responses against Gag P24 and to subdominant antigens such as Pol-1 and Pol-2 in mice vaccinated with HIV conserved sequence as a whole showed enhanced responses compared to A*0201 peptide specific responses. Very minimal to no responses were seen against specific peptide stimulations in Ad5/35 backbone vector primed mice indicating specificity of responses in antigen vaccinated mice. The data indicates that the responses in A*0201 transgenic mice were likely driven by C57/BL6 background and the endogenic processing and presentation of A*0201 specific epitopes on HLA A*0201 allele is not optimal (FIG. 35A-35D).

### Example 10

### Inducing T Cell Responses with Arenavirus Vectors

In this example, we evaluated approaches for inducing T cell responses following immunization with arenavirus vectors. To do this, we evaluated the immunogenicity of the viral vectors in mouse and non-human primates and describe the magnitude and phenotypic characteristics of the antigen specific responses elicited by immunization.

Construction of arenavirus viral vectors expressing SIV antigens. Adenovirus (Ad5/35 or Ad5) or arenavirus vectors expressing optimally defined SIV full length proteins were designed. Sequences from the SIV sme543 Gag strain (SIV SME543; Genbank Sequence ID: U72748) were used to develop a construct with mammalian codon codon-bias. SIVsme543 Pol construct was developed with the following inactivating mutations introduced: deletion of DTG motif in protease, YMDD sequence (SEQ ID NO: 462) in reverse transcriptase, 473E in RNaseH, and D64, D113 and E150 in integrase (see, e.g., Hansen, et al., Nature, 2011. 473(7348):523-7; Kulkarni, et al., Vaccine, 2011. 29(39):6742-54; Loeb, et al., Nature, 1989. 340(6232):397-400; Larder, et al., Nature, 1987. 327(6124):716-7; Schatz, et al., FEBS Lett, 1989. 257(2):311-4; and Leavitt, et al., J Biol Chem, 1993. 268(3):2113-9). Due to package insert limitations in the arenavirus the Pol vector was divided into two segments of Pol-1 (protease and reverse transcriptase) and Pol-2 (RNAse H and Integrase). The SIV env sequence includes a truncated gp41. Expression plasmids and viral vectors were synthesized as previously described; 14 vectors in tri-segmented replication attenuated or bi-segmented replication defective arenavirus platforms with either a Lymphocytic choriomeningitis mammarenavirus (LCMV) or Cali mammarenavirus (a.k.a., Pichinde mammarenavirus or Pichinde arenavirus) (PICV) vector backbone were generated. Replication defective arenavirus vectors used are described in WO 2009/083210. Replication attenuated arenavirus vectors used are described in WO 2016075250 (LCMV) and WO 2017/198726 (Pichinde).

Immunizations. Ten-week-old C57BL/6 mice were immunized with either 1×10⁵ RCV FFU for replication attenuated or 1×10⁶ RCV FFU for replication defective per antigen of LCMV or PICV vectors expressing SIV immunogens by intravenous (i.v.) injections. The vaccine vector was administered in a volume of 200 μl formulated in buffer (10 mM Hepes, 150 mM NaCl, 20 mM Glycine, pH 7.4 (+0.2). For stabilization 10% Sorbitol was added. Mice were anesthetized with isoflurane prior to vaccine immunization. Animals were housed at the WuXi AppTec (Shanghai, China) and experiments were performed in accordance with the regulations of the Association for Assessment and Accreditation of Laboratory Animal Care (AAALAC).

Homologous prime boost. Mice were primed with 1×10⁵ RCV FFU for replication attenuated and 1×10⁶ FFU for replication defective arenavirus vectors either LCMV or PICV expressing SIV immunogens by intravenous (i.v.) injections in the tail vein and rested for 21 days before homologous boost with vectors expressing identical antigens. Immunogenicity and cellular phenotype were evaluated by analyzing splenocytes by ELISpot assay as previously described (Miyahira, et al., J Immunol Methods, 1995. 181(1):45-54), intracellular cytokine staining (ICS) or tetramer staining at various time points, typically on day 7 post prime or day 26 post-boost.

Heterologous prime boost. Mice were primed with 1×10⁵ RCV FFU for replication attenuated and 1×10⁶ FFU for replication defective arenavirus vectors either LCMV or PICV expressing SIV immunogens by intravenous (i.v.) injections in the tail vein and rested for 21 days before heterologous boost with vectors expressing identical antigens. If the initial prime was LCMV the heterologous boost was PICV, and vice versa. Immunogenicity and cellular phenotype were evaluated by analyzing splenocytes by ELISpot assay as previously described (Miyahira, et al., supra), ICS or tetramer staining at various time points, typically on day 7 post prime or day 26 post-boost.

### Results

In a single vector prime with a highly immunogenic antigen (SIV gp-140) we observed enhanced priming with LCMV compared to PICV in the same tri-segmented replication attenuated platform (e.g., as described in WO 2016075250 (LCMV) and WO 2017/198726 (Pichinde)). In a prime-boost immunization schedule we observed that a heterologous prime-boost significantly enhanced immunogenicity. This was most evident with less immunogenic antigens such as Pol-1 and Pol-2. No significant difference was observed with vectors expressing gp-140, however, this may reflect saturation of the IFN-γ ELISpot assay. In heterologous prime-boost comparing immunization with replication attenuated arenavirus vectors to replication defective arenavirus vectors, we observed significant enhancement of immunogenicity following multi-vector immunization with the replication attenuated arenavirus vectors compared with replication defective arenavirus vectors.

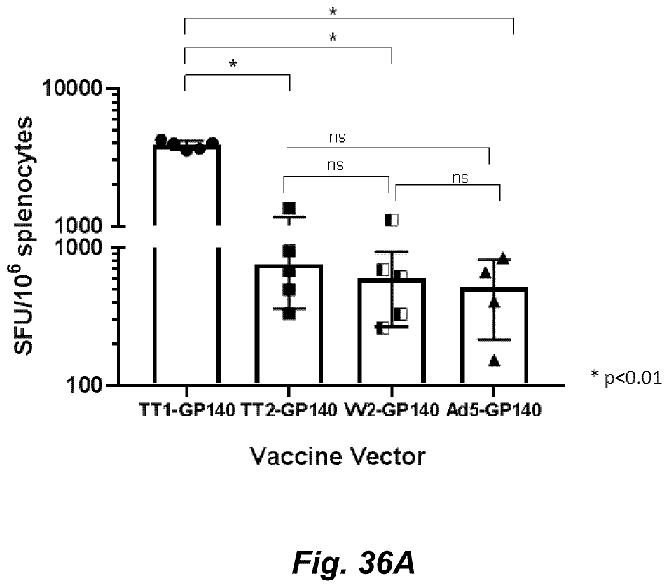

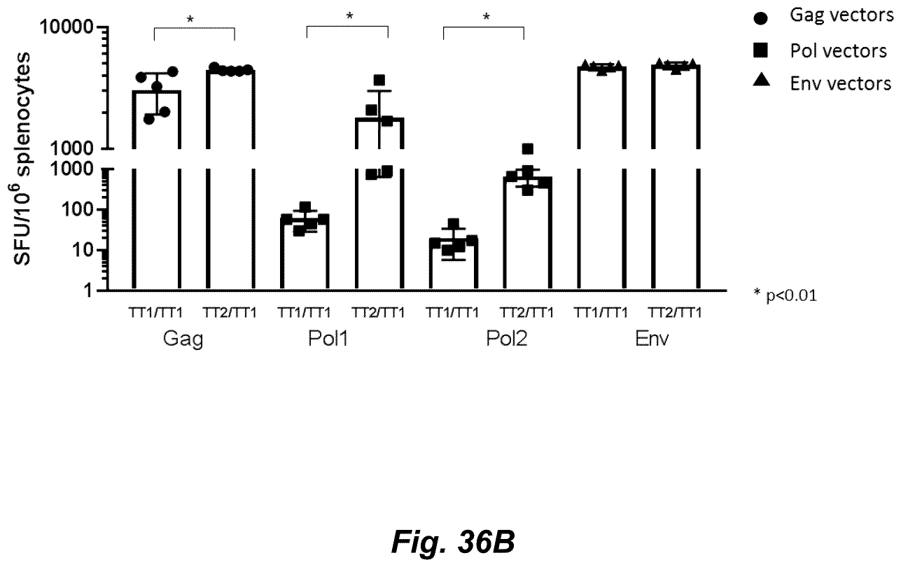

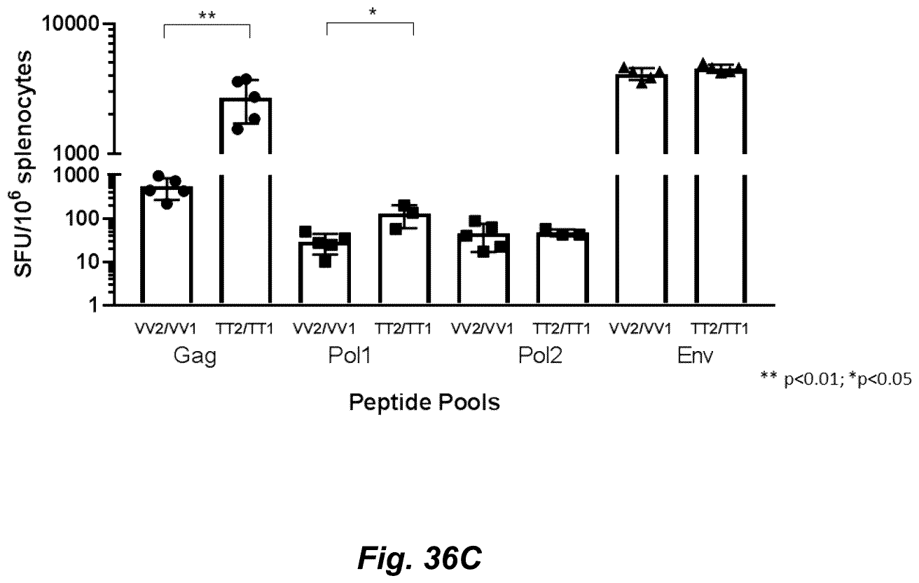

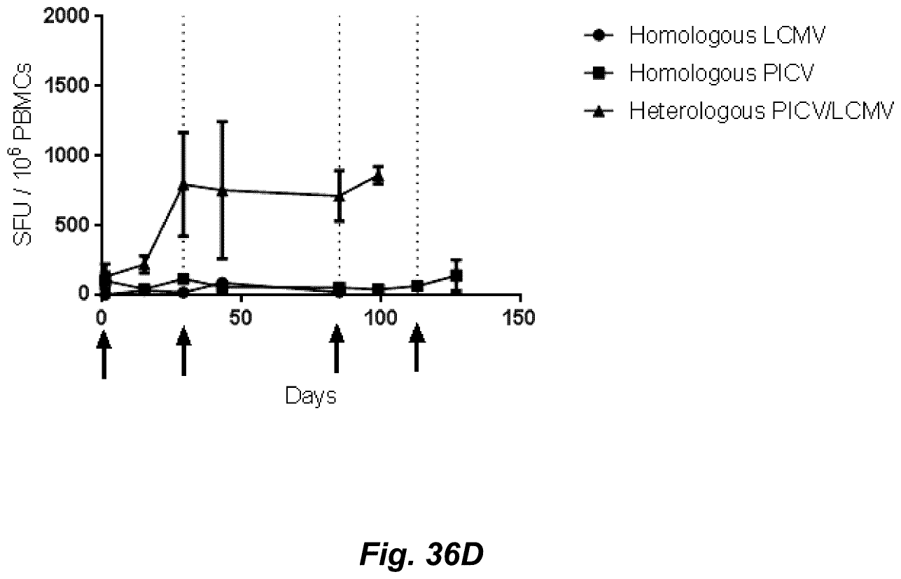

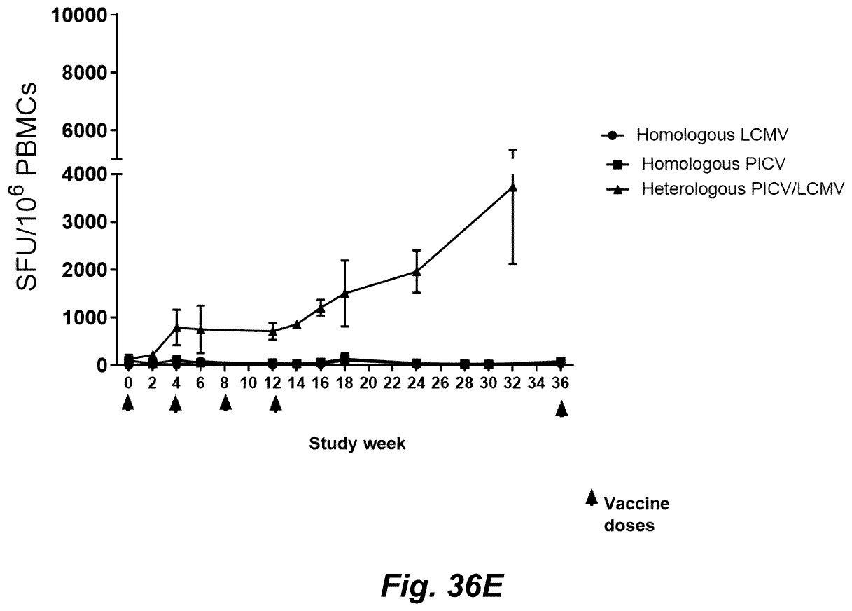

Enhanced immunogenicity following heterologous prime boost was also confirmed following immunization of non-human primates. These data are consistent with the conclusion that arenavirus vectors expressing SIV/HIV antigens heterologous prime boost enhance immunogenicity. The results are shown in FIGS. 36A-36E.

### Example 11

### Mapping Vaccine Specific Epitope Responses

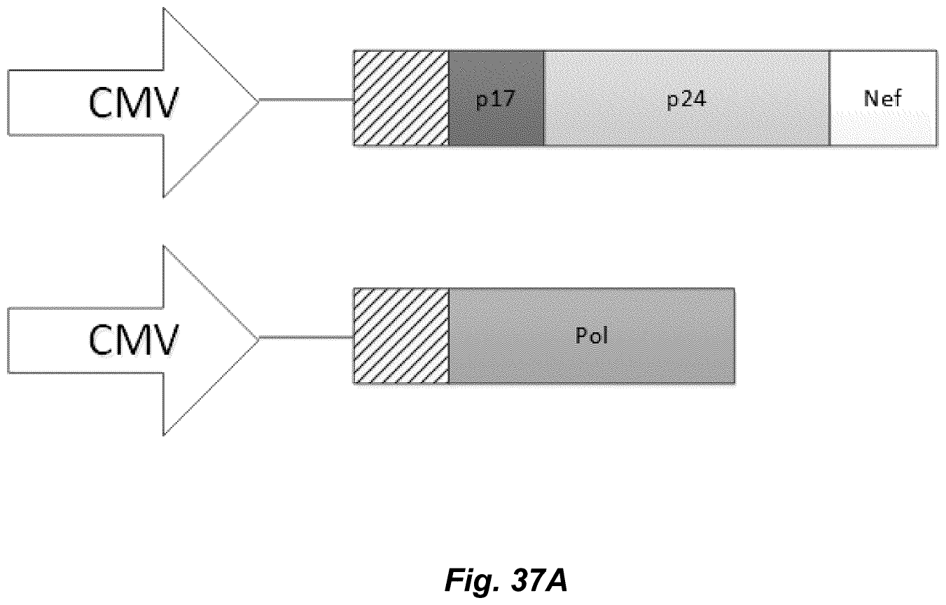

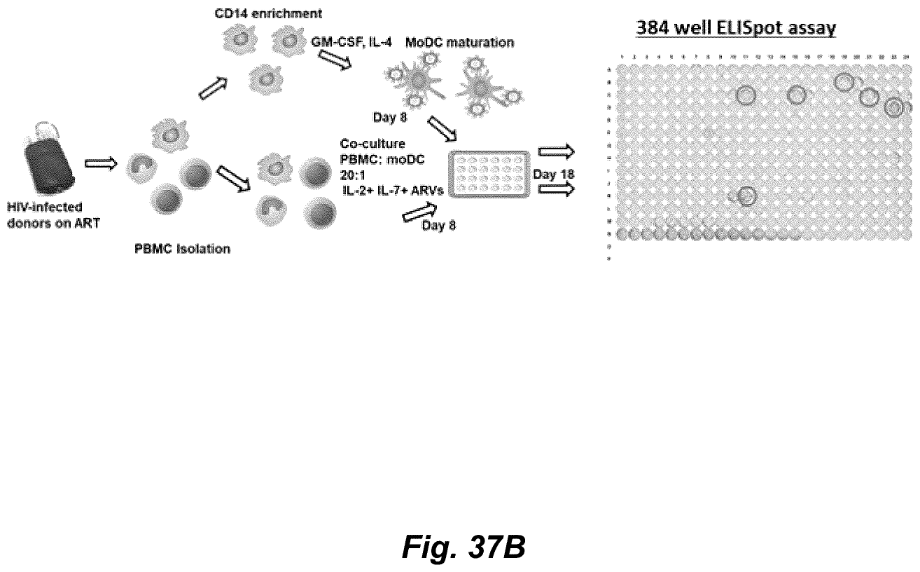

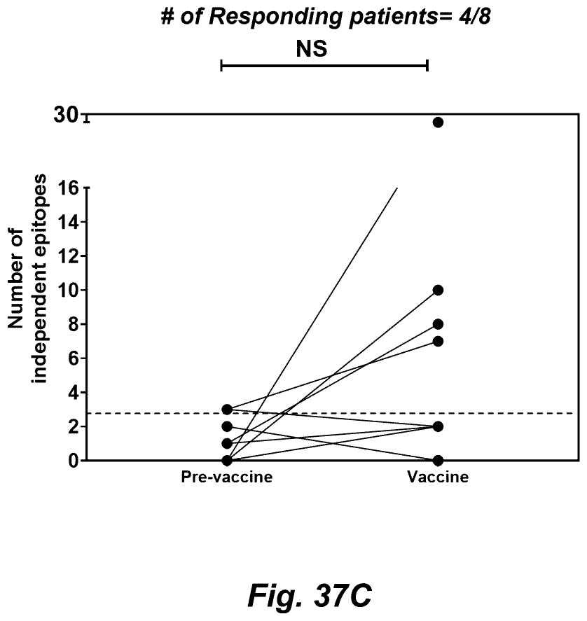

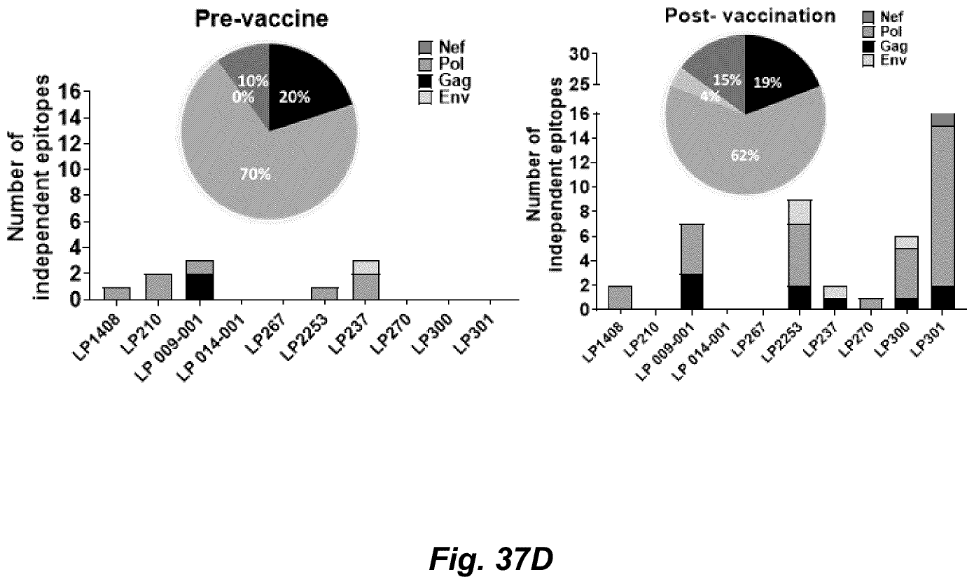

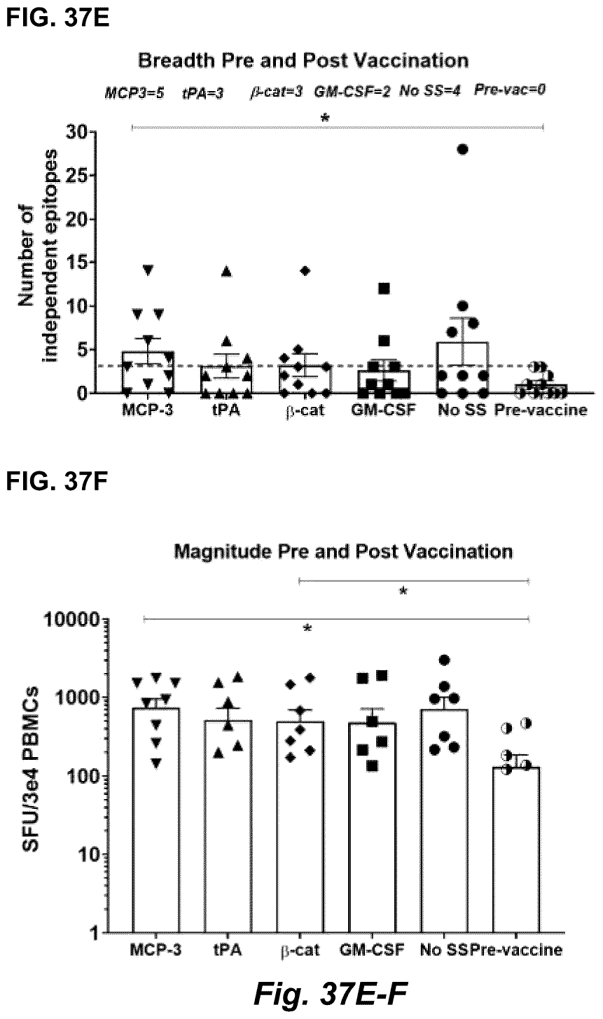

In this example, we used the in vitro T cell priming assay to decode the CD8+ T cell responses to the vaccine immunogen. We focused on determining the epitopes within conserved regions vaccine that induce antigen specific T cell responses and evaluated the impact of pre-existing responses on induction of de novo responses (original antigenic sin). In addition, we also determined if modifications to the immunogen e.g., addition of signal sequences, can modify the breadth of T cell responses generated. Fusion proteins of SEQ ID NOs: 353, 354, 355, 356, 357, 363, 364, 365, 366 and 429 were used in this assay (FIG. 37A).

### Methods

ELISpot Assays. 384 ELISpot plates (Cellular Technologies Limited) were coated with capture antibody and used for all epitope mapping experiments. Briefly, 3×10⁴ cells from Day 10 moDC-CD8+ T cell/PBMC cultures were seeded to each well. Individual 15-mer peptide pools overlapping by 11 amino acids spanning the entire HIV conserved regions were added to each well and used in IFN-γ ELISpot assays to evaluate vaccine immunogenicity. For positive controls, 50 ng/ml PMA (Sigma) was added. Plates were incubated at 37° C. in 5% CO₂ for 24 hours. After 24 hours stimulation, the cells were removed from the plates and the wells were washed three times in PBS prior to three washes with PBS containing 0.05% tween. Biotinylated anti-IFN-γ detection antibody was then added to the plates for 2 hours at room temperature. The plates were then washed three times with PBS containing 0.05% tween prior to the addition of streptavidin-conjugated alkaline phosphatase (AP). Wells were then washed two times with 0.05% tween-PBS and then two times with distilled water prior to the addition of the blue developer solution. The plates were then incubated at room temperature for 15 minutes before the reaction was stopped using tap water. The wells were then dried overnight and spot forming units (SFUs) were counted on an Immunospot ELISpot reader. The settings were identical for all plates and counts were expressed at SFU per 10⁶ PBMCs. The results are shown in FIGS. 37A-37F.

To determine the epitopes within conserved regions vaccine that induce antigen specific T cell responses, we adopted a 384 well ELISpot assay that utilizes individual 15 mers per well rather than peptide pools (FIG. 37B) and completed this analysis on 10 patient samples with different HLA profiles (Table 3).

TABLE 3
Analysis on Ten Patient Samples with Different HLA Profiles

| | HLA | | | | | | Viral | |
|---|---|---|---|---|---|---|---|---|
| | A 1^{st} allele | A 2^{nd} allele | B 1^{st} allele | B 2^{nd} allele | C 1^{st} allele | C 2^{nd} allele | load | Gender |
| LP1408 | A*0101 | A*0201 | B*0702 | B*4101 | Cw*0702 | Cw*1700 | | |
| LP210 | | | | | | | 60 | |
| LP 009-001 | A*25:01 | A*26:01 | B*27:EKN | B*44:AMUT | C*01:02 | C*05:01 | <50 | M |
| LP-014-001 | A02:ANGA | A*03:01 | B*27:EKN | B*44:AMUT | C*01:02 | C*05:01 | <50 | M |
| LP267 | A*03:01 | A*24:02 | B*18:01 | B*41:01 | C*07:01 | C*17:01 | | |
| LP2253 | A*0101 | A*0301 | B*3501 | B*5201 | Cw*0401 | Cw*1202 | | |
| LP237 | A*0201/02:01L | | B*15:01 | | C*03:04 | | 92 | |
| LP270 | A*03:01 | A*33:01 | B*07:02/61/161N | B*44:03 | C*02:02 | C*07:02 | | |
| LP300 | A*24:01:01 | A*11:01:01 | B*07:02 | B*08:01 | C*07:01 | C*07:02 | <50 | M |
| LP301 | A*03:01 | A*32:01 | B*07 | B*40:01 | C*03:04 | C*07:02 | <50 | M |

EKN = 05/13;
AMUT = 44:02, 44:02S, 44:19N;
ANGA = 02:01/02:0IL

The data are consistent with the conclusion that the conserved regions vaccine constructs expressing Gag-Nef and Pol-Env can prime de novo responses predominantly again Pol epitopes (FIG. 37C-37D). The data further indicates that the presence of signal sequences does not significantly enhance the magnitude or the breadth of the response. However, the presence of MCP-3 may increase the number of responders (defined as ≥3 epitopes recognized given the data from the STEP Trial; see, Janes, et al., J Infect Dis (2013) 208(8):1231-1239; ClinicalTrials.gov identifier: NCT00095576). The results are presented in FIGS. 37E-37F.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A fusion polypeptide comprising an amino acid sequence of any one of SEQ ID NOs: 357-362, or a sequence that is at least 90%, 91%, 92%, 93%, 94%, 95%, 96%, 97%, 98%, or 99% identical to any one of SEQ ID NOs: 357-362, wherein the full-length of the fusion polypeptide comprises polypeptide segments encoded by HIV-1 genes Pol and Env, is at least about 335 amino acids and up to about 970 amino acids, and wherein the fusion polypeptide is capable of inducing, promoting or stimulating proliferation and/or activation of one or more cell types selected from monocyte-derived dendritic cells (DCs), CD8+ T cells and CD4+ T cells.

2-4. (canceled)

5. The fusion polypeptide of claim 1, wherein the fusion polypeptide does not comprise and HIV-1 Pol polypeptide segment comprising the amino acid sequence YMDD (SEQ ID NO: 462) or YVDD (SEQ ID NO: 463).

6-7. (canceled)

8. The fusion polypeptide of claim 1, comprising an N-terminal signal peptide or leader sequence.

9. The fusion polypeptide of claim 8, wherein the signal peptide or leader sequence is from a source protein selected from the group consisting of: colony stimulating factor 2 (CSF2, GM-CSF), tissue type plasminogen activator (PLAT, t-PA), C-C motif chemokine ligand 7 (CCL7, MCP-3), C-X-C motif chemokine ligand 10 (CXCL10, IP-10), catenin beta 1 (CTNNB1), CD74 (p33; DHLAG; HLADG; Ia-GAMMA, invariant chain), serum albumin (ALB), polyubiquitin B/C (UBB/UBC), calreticulin (CALR), vesicular stomatitis virus G protein (VSV-G), lysosomal associated membrane protein 1 (LAMP-1) and lysosomal associated membrane protein 2 (LAMP-2).

10. A polynucleotide encoding one or more fusion polypeptides of claim 1.

11. The polynucleotide of claim 10, wherein the polynucleotide comprises cDNA, mRNA, self-amplifying RNA (SAM), self-replicating RNA, or self-amplifying replicon RNA (RepRNA).

12-14. (canceled)

15. An expression cassette, comprising a polynucleotide of claim 10 operably linked to one or more regulatory sequences.

16-17. (canceled)

18. A vector comprising one or more polynucleotides of claim 10.

19. The vector of claim 18, wherein the vector is a plasmid vector, a bacterial vector or a viral vector.

20-22. (canceled)

23. The vector of claim 18, wherein the viral vector is from a virus from a taxonomical family selected from Adenoviridae, Arenaviridae, Herpesviridae, Poxviridae, Paramyxoviridae, Flaviviridae, Rhabdoviridae, Togaviridae.

24-33. (canceled)

34. A host cell comprising one or more polynucleotides of claim 10.

35-39. (canceled)

40. An immunogenic composition comprising one or more of the fusion polypeptides claim 1, and a pharmaceutically acceptable carrier.

41-45. (canceled)

46. A pharmaceutical composition comprising one or more of the fusion polypeptides of claim 1, and a pharmaceutically acceptable carrier.

47. The pharmaceutical composition of claim 46, comprising two or more fusion polypeptides, two or more polynucleotides or two or more vectors.

48-129. (canceled)

130. The fusion polypeptide of claim 9, comprising an amino acid sequence of any one of SEQ ID NOs: 363-367 or a sequence that is at least 95%, 96%, 97%, 98%, or 99% identical to any one of SEQ ID NOs: 363-367.


SEQUENCE LISTING (519 sequences)

SEQ ID NO: 1 - protein, 25 aa, Human immunodeficiency virus
CSATEKLWVTVYYGVPVWKEATTTL

SEQ ID NO: 2 - protein, 15 aa, Human immunodeficiency virus
LWVTVYYGVPVWKEA

SEQ ID NO: 3 - protein, 15 aa, Human immunodeficiency virus
LWVTIYYGVPVWKDA

SEQ ID NO: 4 - protein, 14 aa, Human immunodeficiency virus
LWVTVYYGVPVWKE

SEQ ID NO: 5 - protein, 14 aa, Human immunodeficiency virus
LWVTIYYGVPVWKD

SEQ ID NO: 6 - protein, 9 aa, Human immunodeficiency virus
VTVYYGVPV

SEQ ID NO: 7 - protein, 9 aa, Human immunodeficiency virus
VTIYYGVPV

SEQ ID NO: 8 - protein, 14 aa, Human immunodeficiency virus
ATTTLFCASDAKAY

SEQ ID NO: 9 - protein, 14 aa, Human immunodeficiency virus
ANTTLFCASDAKGY

SEQ ID NO: 10 - protein, 25 aa, Human immunodeficiency virus
KAYDTEVHNVWATHACVPTDPNPQE

SEQ ID NO: 11 - protein, 19 aa, Human immunodeficiency virus
AHNVWATHACVPTDPNPQE

SEQ ID NO: 12 - protein, 19 aa, Human immunodeficiency virus
VHNIWATHACVPTDPSPQE

SEQ ID NO: 13 - protein, 18 aa, Human immunodeficiency virus
HNVWATHACVPTDPNPQE

SEQ ID NO: 14 - protein, 18 aa, Human immunodeficiency virus
HNIWATHACVPTDPSPQE

SEQ ID NO: 15 - protein, 9 aa, Human immunodeficiency virus
NVWATHACV

SEQ ID NO: 16 - protein, 9 aa, Human immunodeficiency virus
NIWATHACV

SEQ ID NO: 17 - protein, 23 aa, Human immunodeficiency virus
DIISLWDQSLKPCVKLTPLCVTL

SEQ ID NO: 18 - protein, 23 aa, Human immunodeficiency virus
DIISLWDESLKPCVKLTPICVTL

SEQ ID NO: 19 - protein, 25 aa, Human immunodeficiency virus
DQSLKPCVKLTPLCVTLNCTDLRNT

SEQ ID NO: 20 - protein, 25 aa, Human immunodeficiency virus
DESLKPCVKLTPICVTLNCTDLRNT

SEQ ID NO: 21 - protein, 9 aa, Human immunodeficiency virus
KLTPLCVTL

SEQ ID NO: 22 - protein, 9 aa, Human immunodeficiency virus
KLTPICVTL

SEQ ID NO: 23 - protein, 18 aa, Human immunodeficiency virus
SFEPIPIHYCAPAGFAIL

SEQ ID NO: 24 - protein, 18 aa, Human immunodeficiency virus
TFEPIPIHYCTPAGFAIL

SEQ ID NO: 25 - protein, 9 aa, Human immunodeficiency virus
PAGFAILKC

SEQ ID NO: 26 - protein, 9 aa, Human immunodeficiency virus
PAGFALLKC

SEQ ID NO: 27 - protein, 25 aa, Human immunodeficiency virus
GTGPCTNVSTVQCTHGIRPVVSTQL

SEQ ID NO: 28 - protein, 28 aa, Human immunodeficiency virus
NVSTVQCTHGIRPVVSTQLLLNGSLAEE

SEQ ID NO: 29 - protein, 28 aa, Human immunodeficiency virus
NISTVQCTHGIKPVVSTQLLLNGSLAEK

SEQ ID NO: 30 - protein, 9 aa, Human immunodeficiency virus
STVQCTHGI

SEQ ID NO: 31 - protein, 11 aa, Human immunodeficiency virus
FNCGGEFFYCN

SEQ ID NO: 32 - protein, 11 aa, Human immunodeficiency virus
FNCRGEFFYCN

SEQ ID NO: 33 - protein, 10 aa, Human immunodeficiency virus
VGKAMYAPPI

SEQ ID NO: 34 - protein, 10 aa, Human immunodeficiency virus
VGRAMYAPPI

SEQ ID NO: 35 - protein, 10 aa, Human immunodeficiency virus
GGDMRDNWRS

SEQ ID NO: 36 - protein, 10 aa, Human immunodeficiency virus
GGNMKDNWRS

SEQ ID NO: 37 - protein, 15 aa, Human immunodeficiency virus
MRDNWRSELYKYKVV

SEQ ID NO: 38 - protein, 15 aa, Human immunodeficiency virus
MKDNWRSELYRYKVV

SEQ ID NO: 39 - protein, 11 aa, Human immunodeficiency virus
AKRRVVQREKR

SEQ ID NO: 40 - protein, 11 aa, Human immunodeficiency virus
ARRRVVQREKR

SEQ ID NO: 41 - protein, 105 aa, Human immunodeficiency virus
KRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTT

SEQ ID NO: 42 - protein, 105 aa, Human immunodeficiency virus
RRRVVQREKRAIGLGAVFLGFLGTAGSTMGAASMTLTVQARLLLSGIVQQQSNLLRAIEAQQHMLQLTVWGIKQLQARILAVERYLRDQQLLGIWGCSGRLICTT

SEQ ID NO: 43 - protein, 16 aa, Human immunodeficiency virus
FLGFLGAAGSTMGAAS

SEQ ID NO: 44 - protein, 16 aa, Human immunodeficiency virus
FLGFLGTAGSTMGAAA

SEQ ID NO: 45 - protein, 74 aa, Human immunodeficiency virus
ASITLTVQARQLLSGIVQQQNNLLRAIEAQQHLLQLTVWGIKQLQARVLAVERYLKDQQLLGIWGCSGKLICTT

SEQ ID NO: 46 - protein, 74 aa, Human immunodeficiency virus
ASMTLTVQARLLLSGIVQQQSNLLRAIEAQQHMLQLTVWGIKQLQARILAVERYLRDQQLLGIWGCSGRLICTT

SEQ ID NO: 47 - protein, 21 aa, Human immunodeficiency virus
TLTVQARQLLSGIVQQQNNLL

SEQ ID NO: 48 - protein, 21 aa, Human immunodeficiency virus
TLTVQARLLLSGIVQQQSNLL

SEQ ID NO: 49 - protein, 11 aa, Human immunodeficiency virus
NLLRAIEAQQH

SEQ ID NO: 50 - protein, 11 aa, Human immunodeficiency virus
NLLKAIEAQQH

SEQ ID NO: 51 - protein, 27 aa, Human immunodeficiency virus
AIEAQQHLLQLTVWGIKQLQARVLAVE

SEQ ID NO: 52 - protein, 27 aa, Human immunodeficiency virus
AIEAQQHMLQLTVWGIKQLQARILAVE

SEQ ID NO: 53 - protein, 9 aa, Human immunodeficiency virus
ERYLKDQQL

SEQ ID NO: 54 - protein, 9 aa, Human immunodeficiency virus
ERYLRDQQL

SEQ ID NO: 55 - protein, 9 aa, Human immunodeficiency virus
YLKDQQLLG

SEQ ID NO: 56 - protein, 9 aa, Human immunodeficiency virus
YLRDQQLLG

SEQ ID NO: 57 - protein, 25 aa, Human immunodeficiency virus
YLKDQQLLGIWGCSGKLICTTAVPW

SEQ ID NO: 58 - protein, 18 aa, Human immunodeficiency virus
DQQLLGIWGCSGKLICTT

SEQ ID NO: 59 - protein, 18 aa, Human immunodeficiency virus
DQQLLGLWGCSGKLICPT

SEQ ID NO: 60 - protein, 9 aa, Human immunodeficiency virus
GIWGCSGKL

SEQ ID NO: 61 - protein, 9 aa, Human immunodeficiency virus
GLWGCSGKL

SEQ ID NO: 62 - protein, 11 aa, Human immunodeficiency virus
WLWYIKIFIMI

SEQ ID NO: 63 - protein, 11 aa, Human immunodeficiency virus
WLWYIRIFIMI

SEQ ID NO: 64 - protein, 14 aa, Human immunodeficiency virus
IFIMIVGGLIGLRI

SEQ ID NO: 65 - protein, 14 aa, Human immunodeficiency virus
LFIMIVGGLVGLRI

SEQ ID NO: 66 - protein, 15 aa, Human immunodeficiency virus
VNRVRQGYSPLSFQT

SEQ ID NO: 67 - protein, 15 aa, Human immunodeficiency virus
VNRVRKGYSPLSFQI

SEQ ID NO: 68 - protein, 11 aa, Human immunodeficiency virus
MGARASVLSGG

SEQ ID NO: 69 - protein, 11 aa, Human immunodeficiency virus
MGARASILSGG

SEQ ID NO: 70 - protein, 53 aa, Human immunodeficiency virus
MGARASVLSGGELDRWEKIRLRPGGKKKYRLKHIVWASRELERFAVNPGLLET

SEQ ID NO: 71 - protein, 53 aa, Human immunodeficiency virus
MGARASILSGGKLDKWEKIRLRPGGRKKYKLKHIVWASRELERFAVNPGLLET

SEQ ID NO: 72 - protein, 13 aa, Human immunodeficiency virus
LDRWEKIRLRPGG

SEQ ID NO: 73 - protein, 13 aa, Human immunodeficiency virus
LDKWEKIRLRPMG

SEQ ID NO: 74 - protein, 9 aa, Human immunodeficiency virus
IRLRPGGKK

SEQ ID NO: 75 - protein, 9 aa, Human immunodeficiency virus
IRLRPGGRK

SEQ ID NO: 76 - protein, 23 aa, Human immunodeficiency virus
LKHIVWASRELERFAVNPGLLET

SEQ ID NO: 77 - protein, 23 aa, Human immunodeficiency virus
LKHLVWASRELERFALNPGLLET

SEQ ID NO: 78 - protein, 15 aa, Human immunodeficiency virus
ASRELERFAVNPGLL

SEQ ID NO: 79 - protein, 15 aa, Human immunodeficiency virus
ASRELERFALNPGLL

SEQ ID NO: 80 - protein, 9 aa, Human immunodeficiency virus
TGSEELKSL

SEQ ID NO: 81 - protein, 9 aa, Human immunodeficiency virus
TGSEELRSL

SEQ ID NO: 82 - protein, 9 aa, Human immunodeficiency virus
DTKEALDKI

SEQ ID NO: 83 - protein, 9 aa, Human immunodeficiency virus
DTKEALEKI

SEQ ID NO: 84 - protein, 9 aa, Human immunodeficiency virus
EALDKIEEE

SEQ ID NO: 85 - protein, 9 aa, Human immunodeficiency virus
EALEKIEEE

SEQ ID NO: 86 - protein, 10 aa, Human immunodeficiency virus
VSQNYPIVQN

SEQ ID NO: 87 - protein, 10 aa, Human immunodeficiency virus
VSQNFPIVQN

SEQ ID NO: 88 - protein, 231 aa, Human immunodeficiency virus
PIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVL

SEQ ID NO: 89 - protein, 231 aa, Human immunodeficiency virus
PIVQNIQGQMVHQPISPRTLNAWVKVIEEKAFSPEVIPMFTALSEGATPHDLNTMLNTIGGHQAAMQMLKDTINEEAAEWDRVHPVHAGPVAPGQMRDPRGSDIAGTTSNLQEQIGWMTSNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVL

SEQ ID NO: 90 - protein, 25 aa, Human immunodeficiency virus
MVHQAISPRTLNAWVKVVEEKAFSP

SEQ ID NO: 91 - protein, 25 aa, Human immunodeficiency virus
MVHQPISPRTLNAWVKVIEEKAFSP

SEQ ID NO: 92 - protein, 71 aa, Human immunodeficiency virus
ISPRTLNAWVKVVEEKAFSPEVIPMFSALSEGATPQDLNTMLNTVGGHQAAMQMLKETINEEAAEWDRLHP

SEQ ID NO: 93 - protein, 71 aa, Human immunodeficiency virus
LSPRTLNAWVKVIEEKAFSPEVIPMFTALSEGATPHDLNTMLNTIGGHQAAMQMLKDTINEEAAEWDRVHP

SEQ ID NO: 94 - protein, 223 aa, Human immunodeficiency virus
ISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQ

SEQ ID NO: 95 - protein, 223 aa, Human immunodeficiency virus
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQ

SEQ ID NO: 96 - protein, 9 aa, Human immunodeficiency virus
RTLNAWVKV

SEQ ID NO: 97 - protein, 25 aa, Human immunodeficiency virus
LSEGATPQDLNTMLNTVGGHQAAMQ

SEQ ID NO: 98 - protein, 25 aa, Human immunodeficiency virus
LSEGATPHDLNTMLNTIGGHQAAMQ

SEQ ID NO: 99 - protein, 9 aa, Human immunodeficiency virus
DLNTMLNTV

SEQ ID NO: 100 - protein, 9 aa, Human immunodeficiency virus
DLNTMLNTI

SEQ ID NO: 101 - protein, 27 aa, Human immunodeficiency virus
PGQMREPRGSDIAGTTSTLQEQIGWMT

SEQ ID NO: 102 - protein, 27 aa, Human immunodeficiency virus
PGQMRDPRGSDIAGSTSTLQEQIAWMT

SEQ ID NO: 103 - protein, 33 aa, Human immunodeficiency virus
NPPIPVGEIYKRWIILGLNKIVRMYSPTSILDI

SEQ ID NO: 104 - protein, 33 aa, Human immunodeficiency virus
NPPIPVGDIYKRWIIMGLNKIVRMYSPVSILDI

SEQ ID NO: 105 - protein, 34 aa, Human immunodeficiency virus
PVGEIYKRWIILGLNKIVRMYSPTSILDIRQGPK

SEQ ID NO: 106 - protein, 34 aa, Human immunodeficiency virus
PVGDIYKRWIIMGLNKIVRMYSPVSILDIKQGPK

SEQ ID NO: 107 - protein, 18 aa, Human immunodeficiency virus
WIILGLNKIVRMYSPTSI

SEQ ID NO: 108 - protein, 18 aa, Human immunodeficiency virus
WIIMGLNKIVRMYSPVSI

SEQ ID NO: 109 - protein, 34 aa, Human immunodeficiency virus
SILDIRQGPKEPFRDYVDRFYKTLRAEQASQEVK

SEQ ID NO: 110 - protein, 34 aa, Human immunodeficiency virus
SILDIKQGPKEPFRDYVDRFYRTLRAEQASQDVK

SEQ ID NO: 111 - protein, 34 aa, Human immunodeficiency virus
GPKEPFRDYVDRFYKTLRAEQASQEVKNWMTETL

SEQ ID NO: 112 - protein, 34 aa, Human immunodeficiency virus
GPKEPFRDYVDRFYRTLRAEQASQDVKNWMTETL

SEQ ID NO: 113 - protein, 18 aa, Human immunodeficiency virus
YVDRFYKTLRAEQASQEV

SEQ ID NO: 114 - protein, 18 aa, Human immunodeficiency virus
YVDRFYRTLRAEQASQDV

SEQ ID NO: 115 - protein, 59 aa, Human immunodeficiency virus
QEVKNWMTETLLVQNANPDCKTILKALGPAATLEEMMTACQGVGGPGHKARVLAEAMSQ

SEQ ID NO: 116 - protein, 59 aa, Human immunodeficiency virus
QDVKNWMTETLLVQNSNPDCKTILKALGPGATLEEMMSACQGVGGPSHKARVLAEAMCQ

SEQ ID NO: 117 - protein, 25 aa, Human immunodeficiency virus
ILKALGPAATLEEMMTACQGVGGPG

SEQ ID NO: 118 - protein, 25 aa, Human immunodeficiency virus
ILKALGPGATLEEMMSACQGVGGPS

SEQ ID NO: 119 - protein, 25 aa, Human immunodeficiency virus
LGPAATLEEMMTACQGVGGPGHKAR

SEQ ID NO: 120 - protein, 25 aa, Human immunodeficiency virus
LGPGATLEEMMSACQGVGGPSHKAR

SEQ ID NO: 121 - protein, 9 aa, Human immunodeficiency virus
ATLEEMMTA

SEQ ID NO: 122 - protein, 9 aa, Human immunodeficiency virus
ATLEEMMSA

SEQ ID NO: 123 - protein, 9 aa, Human immunodeficiency virus
EMMTACQGV

SEQ ID NO: 124 - protein, 9 aa, Human immunodeficiency virus
EMMSACQGV

SEQ ID NO: 125 - protein, 10 aa, Human immunodeficiency virus
KCFNCGKEGH

SEQ ID NO: 126 - protein, 10 aa, Human immunodeficiency virus
KCFNCGREGH

SEQ ID NO: 127 - protein, 9 aa, Human immunodeficiency virus
ARNCRAPRK

SEQ ID NO: 128 - protein, 9 aa, Human immunodeficiency virus
AKNCRAPRK

SEQ ID NO: 129 - protein, 39 aa, Human immunodeficiency virus
ARNCRAPRKKGCWKCGKEGHQMKDCTERQANFLGKIWPS

SEQ ID NO: 130 - protein, 39 aa, Human immunodeficiency virus
AKNCRAPRKRGCWKCGREGHQMKDCNERQANFLGKVWPS

SEQ ID NO: 131 - protein, 14 aa, Human immunodeficiency virus
NCRAPRKKGCWKCG

SEQ ID NO: 132 - protein, 14 aa, Human immunodeficiency virus
NCRAPRKRGCWKCG

SEQ ID NO: 133 - protein, 19 aa, Human immunodeficiency virus
GCWKCGKEGHQMKDCTERQ

SEQ ID NO: 134 - protein, 19 aa, Human immunodeficiency virus
GCWKCGREGHQMKDCNERQ

SEQ ID NO: 135 - protein, 17 aa, Human immunodeficiency virus
KDCTERQANFLGKIWPS

SEQ ID NO: 136 - protein, 17 aa, Human immunodeficiency virus
KDCNERQANFLGKVWPS

SEQ ID NO: 137 - protein, 16 aa, Human immunodeficiency virus
RQANFLGKIWPSHKGR

SEQ ID NO: 138 - protein, 16 aa, Human immunodeficiency virus
RQANFLGKVWPSHNGR

SEQ ID NO: 139 - protein, 12 aa, Human immunodeficiency virus
KGRPGNFLQSRP

SEQ ID NO: 140 - protein, 12 aa, Human immunodeficiency virus
NGRPGNFLQNRP

SEQ ID NO: 141 - protein, 10 aa, Human immunodeficiency virus
SLRSLFGNDP

SEQ ID NO: 142 - protein, 10 aa, Human immunodeficiency virus
SLKSLFGNDP

SEQ ID NO: 143 - protein, 9 aa, Human immunodeficiency virus
SLFGNDPSS

SEQ ID NO: 144 - protein, 9 aa, Human immunodeficiency virus
SLFGNDPLS

SEQ ID NO: 145 - protein, 33 aa, Human immunodeficiency virus
LKHIVWASRELERFAVNPGLLETVSQNYPIVQN

SEQ ID NO: 146 - protein, 33 aa, Human immunodeficiency virus
LKHLVWASRELERFALNPGLLETVSQNFPIVQN

SEQ ID NO: 147 - protein, 9 aa, Human immunodeficiency virus
GVGAVSRDL

SEQ ID NO: 148 - protein, 9 aa, Human immunodeficiency virus
GVGAASRDL

SEQ ID NO: 149 - protein, 19 aa, Human immunodeficiency virus
EEVGFPVRPQVPLRPMTYK

SEQ ID NO: 150 - protein, 19 aa, Human immunodeficiency virus
EEVGFPVKPQVPLRPMTFK

SEQ ID NO: 151 - protein, 36 aa, Human immunodeficiency virus
EEVGFPVKPQVPLRPMTFKGALDLSHFLREKGGLEG

SEQ ID NO: 152 - protein, 36 aa, Human immunodeficiency virus
EEVGFPVRPQVPLRPMTYKGALDLSHFLKEKGGLEG

SEQ ID NO: 153 - protein, 22 aa, Human immunodeficiency virus
YKAAVDLSHFLREKGGLEGAAY

SEQ ID NO: 154 - protein, 22 aa, Human immunodeficiency virus
YKGALDLSHFLKEKGGLEGAAY

SEQ ID NO: 155 - protein, 10 aa, Human immunodeficiency virus
SHFLKEKGGL

SEQ ID NO: 156 - protein, 10 aa, Human immunodeficiency virus
SHFLREKGGL

SEQ ID NO: 157 - protein, 9 aa, Human immunodeficiency virus
LKEKGGLEG

SEQ ID NO: 158 - protein, 9 aa, Human immunodeficiency virus
LREKGGLEG

SEQ ID NO: 159 - protein, 16 aa, Human immunodeficiency virus
TQGYFPDWQNYTPGPG

SEQ ID NO: 160 - protein, 16 aa, Human immunodeficiency virus
TQGFFPDWQNYTPEPG

SEQ ID NO: 161 - protein, 32 aa, Human immunodeficiency virus
TQGFFPDWQNYTPEPGIRFPLTFGWCFKLVPL

SEQ ID NO: 162 - protein, 32 aa, Human immunodeficiency virus
TQGYFPDWQNYTPGPGTRYPLTFGWCFKLVPV

SEQ ID NO: 163 - protein, 19 aa, Human immunodeficiency virus
EPGIRFPLTFGWCFKLVPL

SEQ ID NO: 164 - protein, 19 aa, Human immunodeficiency virus
GPGTRYPLTFGWCFKLVPV

SEQ ID NO: 165 - protein, 25 aa, Human immunodeficiency virus
GPGIRYPLLTFGWCFKLPVEPEKVE

SEQ ID NO: 166 - protein, 9 aa, Human immunodeficiency virus
RYPLTFGWC

SEQ ID NO: 167 - protein, 9 aa, Human immunodeficiency virus
RFPLTFGWC

SEQ ID NO: 168 - protein, 15 aa, Human immunodeficiency virus
RYPLTFGWCFKLVPV

SEQ ID NO: 169 - protein, 15 aa, Human immunodeficiency virus
RFPLTFGWCFKLVPL

SEQ ID NO: 170 - protein, 13 aa, Human immunodeficiency virus
PLTFGWCFKLVPV

SEQ ID NO: 171 - protein, 13 aa, Human immunodeficiency virus
PLCFGWCFKLVPL

SEQ ID NO: 172 - protein, 9 aa, Human immunodeficiency virus
LTFGWCFKL

SEQ ID NO: 173 - protein, 9 aa, Human immunodeficiency virus
LCFGWCFKL

SEQ ID NO: 174 - protein, 12 aa, Human immunodeficiency virus
FPQITLWQRPLV

SEQ ID NO: 175 - protein, 12 aa, Human immunodeficiency virus
LPQITLWQRPIV

SEQ ID NO: 176 - protein, 62 aa, Human immunodeficiency virus
FPQITLWQRPLVTIKIGGQLKEALLDTGADDTVLEEMNLPGRWKPKMIGGIGGFIKVRQYDQ

SEQ ID NO: 177 - protein, 62 aa, Human immunodeficiency virus
LPQITLWQRPIVTIKIGGQIKEALLDTGADDTVLEDMNLPGKWKPKMIGGIGGFIKVKQYDQ

SEQ ID NO: 178 - protein, 20 aa, Human immunodeficiency virus
GGQLKEALLDTGADDTVLEE

SEQ ID NO: 179 - protein, 20 aa, Human immunodeficiency virus
GGQIKEALLDTGADDTVLED

SEQ ID NO: 180 - protein, 24 aa, Human immunodeficiency virus
LPGRWKPKMIGGIGGFIKVRQYDQ

SEQ ID NO: 181 - protein, 24 aa, Human immunodeficiency virus
LPGKWKPKMIGGIGGFIKVKQYDQ

SEQ ID NO: 182 - protein, 132 aa, Human immunodeficiency virus
GTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKS

SEQ ID NO: 183 - protein, 132 aa, Human immunodeficiency virus
GTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKS

SEQ ID NO: 184 - protein, 149 aa, Human immunodeficiency virus
GTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDK

SEQ ID NO: 185 - protein, 149 aa, Human immunodeficiency virus
GTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTILDVGDAYFSIPLDK

SEQ ID NO: 186 - protein, 161 aa, Human immunodeficiency virus
GTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPS

SEQ ID NO: 187 - protein, 161 aa, Human immunodeficiency virus
GTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPS

SEQ ID NO: 188 - protein, 192 aa, Human immunodeficiency virus
GTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 189 - protein, 192 aa, Human immunodeficiency virus
GTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMT

SEQ ID NO: 190 - protein, 25 aa, Human immunodeficiency virus
NLLTQIGCTLNFPISPIETVPVKLK

SEQ ID NO: 191 - protein, 25 aa, Human immunodeficiency virus
NLLTQLGCTLNFPISPIDTVPVKLK

SEQ ID NO: 192 - protein, 9 aa, Human immunodeficiency virus
TLNFPISPI

SEQ ID NO: 193 - protein, 24 aa, Human immunodeficiency virus
GLKKKKSVTVLDVGDAYFSVPLDK

SEQ ID NO: 194 - protein, 24 aa, Human immunodeficiency virus
GLKKNKSVTVLDVGDAYFSIPLDK

SEQ ID NO: 195 - protein, 12 aa, Human immunodeficiency virus
DFRKYTAFTIPS

SEQ ID NO: 196 - protein, 12 aa, Human immunodeficiency virus
EFRKYTAFTVPS

SEQ ID NO: 197 - protein, 25 aa, Human immunodeficiency virus
NNETPGIRYQYNVLPQGWKGSPAIF

SEQ ID NO: 198 - protein, 25 aa, Human immunodeficiency virus
NNETPGVRYQYNVLPMGWKGSPAIF

SEQ ID NO: 199 - protein, 30 aa, Human immunodeficiency virus
NNETPGIRYQYNVLPQGWKGSPAIFQSSMT

SEQ ID NO: 200 - protein, 30 aa, Human immunodeficiency virus
NNETPGVRYQYNVLPMGWKGSPAIFQCSMT

SEQ ID NO: 201 - protein, 9 aa, Human immunodeficiency virus
YQYNVLPQG

SEQ ID NO: 202 - protein, 9 aa, Human immunodeficiency virus
YQYNVLPMG

SEQ ID NO: 203 - protein, 9 aa, Human immunodeficiency virus
FQSSMTKIL

SEQ ID NO: 204 - protein, 9 aa, Human immunodeficiency virus
FQCSMTKIL

SEQ ID NO: 205 - protein, 10 aa, Human immunodeficiency virus
SMTKILEPFR

SEQ ID NO: 206 - protein, 10 aa, Human immunodeficiency virus
SMTKILDPFR

SEQ ID NO: 207 - protein, 9 aa, Human immunodeficiency virus
ILEPFRKQN

SEQ ID NO: 208 - protein, 9 aa, Human immunodeficiency virus
ILDPFRKQN

SEQ ID NO: 209 - protein, 25 aa, Human immunodeficiency virus
FRKQNPDIVIYQYMDDLYVGSDLEI

SEQ ID NO: 210 - protein, 25 aa, Human immunodeficiency virus
KQNPDIVIYQYMDDLYVGSDLEIGQ

SEQ ID NO: 211 - protein, 25 aa, Human immunodeficiency virus
NPDIVIYQYMDDLYVGSDLEIGQHR

SEQ ID NO: 212 - protein, 25 aa, Human immunodeficiency virus
NPDIVIYQYVDDLYVGSDLEIEQHR

SEQ ID NO: 213 - protein, 22 aa, Human immunodeficiency virus
IVIYQYMDDLYVGSDLEIGQHR

SEQ ID NO: 214 - protein, 22 aa, Human immunodeficiency virus
IVIYQYVDDLYVGSDLEIEQHR

SEQ ID NO: 215 - protein, 9 aa, Human immunodeficiency virus
VIYQYMDDL

SEQ ID NO: 216 - protein, 9 aa, Human immunodeficiency virus
VIYQYVDDL

SEQ ID NO: 217 - protein, 9 aa, Human immunodeficiency virus
YQYMDDLYV

SEQ ID NO: 218 - protein, 9 aa, Human immunodeficiency virus
YQYVDDLYV

SEQ ID NO: 219 - protein, 9 aa, Human immunodeficiency virus
YMDDLYVGS

SEQ ID NO: 220 - protein, 9 aa, Human immunodeficiency virus
YVDDLYVGS

SEQ ID NO: 221 - protein, 33 aa, Human immunodeficiency virus
WGFTTPDKKHQKEPPFLWMGYELHPDKWTVQPI

SEQ ID NO: 222 - protein, 33 aa, Human immunodeficiency virus
WGLTTPDKKHQKDPPFLWMGYELHPDRWTVQPI

SEQ ID NO: 223 - protein, 65 aa, Human immunodeficiency virus
WGFTTPDKKHQKEPPFLWMGYELHPDKWTVQPIVLPEKDSWTVNDIQKLVGKLNWASQIYPGIKV

SEQ ID NO: 224 - protein, 65 aa, Human immunodeficiency virus
WGLTTPDKKHQKDPPFLWMGYELHPDRWTVQPIELPEKESWTVNDIQKLIGKLNWASQIYAGIKV

SEQ ID NO: 225 - protein, 25 aa, Human immunodeficiency virus
KKHQKEPPFLWMGYELHPDKWTVQP

SEQ ID NO: 226 - protein, 25 aa, Human immunodeficiency virus
KKHQKDPPFLWMGYELHPDRWTVQP

SEQ ID NO: 227 - protein, 25 aa, Human immunodeficiency virus
PPFLWMGYELHPDKWTVQPIVLPEK

SEQ ID NO: 228 - protein, 25 aa, Human immunodeficiency virus
PPFLWMGYELHPDRWTVQPIELPEK

SEQ ID NO: 229 - protein, 9 aa, Human immunodeficiency virus
FLWMGYELH

SEQ ID NO: 230 - protein, 9 aa, Human immunodeficiency virus
ELHPDKWTV

SEQ ID NO: 231 - protein, 9 aa, Human immunodeficiency virus
ELHPDRWTV

SEQ ID NO: 232 - protein, 25 aa, Human immunodeficiency virus
IVLPEKDSWTVNDIQKLVGKLNWAS

SEQ ID NO: 233 - protein, 25 aa, Human immunodeficiency virus
IELPEKESWTVNDIQKLIGKLNWAS

SEQ ID NO: 234 - protein, 25 aa, Human immunodeficiency virus
VLPEKDSWTVNDIQKLVGKLNWASQ

SEQ ID NO: 235 - protein, 25 aa, Human immunodeficiency virus
ELPEKESWTVNDIQKLIGKLNWASQ

SEQ ID NO: 236 - protein, 31 aa, Human immunodeficiency virus
LPEKDSWTVNDIQKLVGKLNWASQIYPGIKV

SEQ ID NO: 237 - protein, 31 aa, Human immunodeficiency virus
LPEKESWTVNDIQKLIGKLNWASQIYAGIKV

SEQ ID NO: 238 - protein, 25 aa, Human immunodeficiency virus
SWTVNDIQKLVGKLNWASQIYPGIK

SEQ ID NO: 239 - protein, 25 aa, Human immunodeficiency virus
SWTVNDIQKLIGKLNWASQIYAGIK

SEQ ID NO: 240 - protein, 9 aa, Human immunodeficiency virus
WTVNDIQKL

SEQ ID NO: 241 - protein, 9 aa, Human immunodeficiency virus
TVNDIQKLV

SEQ ID NO: 242 - protein, 9 aa, Human immunodeficiency virus
TVNDIQKLI

SEQ ID NO: 243 - protein, 9 aa, Human immunodeficiency virus
KLVGKLNWA

SEQ ID NO: 244 - protein, 9 aa, Human immunodeficiency virus
KLIGKLNWA

SEQ ID NO: 245 - protein, 9 aa, Human immunodeficiency virus
LCKLLRGTK

SEQ ID NO: 246 - protein, 9 aa, Human immunodeficiency virus
LCKLLRGAK

SEQ ID NO: 247 - protein, 19 aa, Human immunodeficiency virus
EAELELAENREILKEPVHG

SEQ ID NO: 248 - protein, 19 aa, Human immunodeficiency virus
EAEIELAENREILREPVHG

SEQ ID NO: 249 - protein, 12 aa, Human immunodeficiency virus
EPVHGVYYDPSK

SEQ ID NO: 250 - protein, 12 aa, Human immunodeficiency virus
EPVHGAYYDPSK

SEQ ID NO: 251 - protein, 22 aa, Human immunodeficiency virus
GQWTYQIYQEPFKNLKTGKYAR

SEQ ID NO: 252 - protein, 22 aa, Human immunodeficiency virus
GQWSYQIYQEPYKNLKTGKYAK

SEQ ID NO: 253 - protein, 16 aa, Human immunodeficiency virus
AHTNDVKQLTEAVQKI

SEQ ID NO: 254 - protein, 16 aa, Human immunodeficiency virus
AHTNDVRQLTEAVQKV

SEQ ID NO: 255 - protein, 10 aa, Human immunodeficiency virus
IVIWGKTPKF

SEQ ID NO: 256 - protein, 10 aa, Human immunodeficiency virus
IVIWGKIPKF

SEQ ID NO: 257 - protein, 13 aa, Human immunodeficiency virus
PKFKLPIQKETWE

SEQ ID NO: 258 - protein, 13 aa, Human immunodeficiency virus
PKFRLPIQKETWD

SEQ ID NO: 259 - protein, 65 aa, Human immunodeficiency virus
PKFKLPIQKETWETWWTEYWQATWIPEWEFVNTPPLVKLWYQLEKEPIVGAETFYVDGAANRETK

SEQ ID NO: 260 - protein, 65 aa, Human immunodeficiency virus
PKFRLPIQKETWDTWWTDYWQATWIPEWEFTNTPPLVKLWYQLETEPIAGVETFYVDGASNRETK

SEQ ID NO: 261 - protein, 25 aa, Human immunodeficiency virus
WETWWTEYWQATWIPEWEFVNTPPL

SEQ ID NO: 262 - protein, 25 aa, Human immunodeficiency virus
WDTWWTDYWQATWIPEWEFTNTPPL

SEQ ID NO: 263 - protein, 31 aa, Human immunodeficiency virus
EYWQATWIPEWEFVNTPPLVKLWYQLEKEPI

SEQ ID NO: 264 - protein, 31 aa, Human immunodeficiency virus
DYWQATWIPEWEFTNTPPLVKLWYQLETEPI

SEQ ID NO: 265 - protein, 9 aa, Human immunodeficiency virus
WQATWIPEW

SEQ ID NO: 266 - protein, 16 aa, Human immunodeficiency virus
GAETFYVDGAANRETK

SEQ ID NO: 267 - protein, 16 aa, Human immunodeficiency virus
GVETFYVDGASNRETK

SEQ ID NO: 268 - protein, 13 aa, Human immunodeficiency virus
TDTTNQKTELQAI

SEQ ID NO: 269 - protein, 13 aa, Human immunodeficiency virus
ADTTNQKTELHAI

SEQ ID NO: 270 - protein, 9 aa, Human immunodeficiency virus
AIHLALQDS

SEQ ID NO: 271 - protein, 9 aa, Human immunodeficiency virus
AIYLALQDS

SEQ ID NO: 272 - protein, 33 aa, Human immunodeficiency virus
LALQDSGLEVNIVTDSQYALGIIQAQPDKSESE

SEQ ID NO: 273 - protein, 33 aa, Human immunodeficiency virus
LALQDSGSEVNIVTDSQYAIGIIQAQPDRSESE

SEQ ID NO: 274 - protein, 25 aa, Human immunodeficiency virus
QDSGLEVNIVTDSQYALGIIQAQPD

SEQ ID NO: 275 - protein, 25 aa, Human immunodeficiency virus
QDSGSEVNIVTDSQYAIGIIQAQPD

SEQ ID NO: 276 - protein, 9 aa, Human immunodeficiency virus
IVTDSQYAL

SEQ ID NO: 277 - protein, 9 aa, Human immunodeficiency virus
IVTDSQYAI

SEQ ID NO: 278 - protein, 9 aa, Human immunodeficiency virus
VSQIIEQLI

SEQ ID NO: 279 - protein, 9 aa, Human immunodeficiency virus
VNQIIEQLI

SEQ ID NO: 280 - protein, 10 aa, Human immunodeficiency virus
QIIEQLIKKE

SEQ ID NO: 281 - protein, 10 aa, Human immunodeficiency virus
QIIEQLINKE

SEQ ID NO: 282 - protein, 26 aa, Human immunodeficiency virus
KEKVYLAWVPAHKGIGGNEQVDKLVS

SEQ ID NO: 283 - protein, 26 aa, Human immunodeficiency virus
KEKIYLAWVPAHKGIGGNEQIDKLVS

SEQ ID NO: 284 - protein, 16 aa, Human immunodeficiency virus
GIRKVLFLDGIDKAQE

SEQ ID NO: 285 - protein, 16 aa, Human immunodeficiency virus
GIRRVLFLDGIEKAQD

SEQ ID NO: 286 - protein, 9 aa, Human immunodeficiency virus
HEKYHSNWR

SEQ ID NO: 287 - protein, 9 aa, Human immunodeficiency virus
HEKYHNNWR

SEQ ID NO: 288 - protein, 9 aa, Human immunodeficiency virus
MASDFNLPP

SEQ ID NO: 289 - protein, 9 aa, Human immunodeficiency virus
MASDFNIPP

SEQ ID NO: 290 - protein, 13 aa, Human immunodeficiency virus
FNLPPVVAKEIVA

SEQ ID NO: 291 - protein, 13 aa, Human immunodeficiency virus
FNLPPIVAKEIVA

SEQ ID NO: 292 - protein, 87 aa, Human immunodeficiency virus
FNLPPVVAKEIVASCDKCQLKGEAMHGQVDCSPGIWQLDCTHLEGKIILVAVHVASGYIEAEVIPAETGQETAYFLLKLAGRWPVKT

SEQ ID NO: 293 - protein, 87 aa, Human immunodeficiency virus
FNLPPIVAKEIVACCDKCQLKGEAIHGQVDCSPGVWQLDCTHLEGKVILVAVHVASGYIEAEIIPTETGQETAYFILKLAGRWPVTT

SEQ ID NO: 294 - protein, 81 aa, Human immunodeficiency virus
VAKEIVASCDKCQLKGEAMHGQVDCSPGIWQLDCTHLEGKIILVAVHVASGYIEAEVIPAETGQETAYFLLKLAGRWPVKT

SEQ ID NO: 295 - protein, 81 aa, Human immunodeficiency virus
VAKEIVACCDKCQLKGEAIHGQVDCSPGVWQLDCTHLEGKVILVAVHVASGYMEAEVIPTETGQETAYFILKLAGRWPVTT

SEQ ID NO: 296 - protein, 25 aa, Human immunodeficiency virus
QLKGEAMHGQVDCSPGIWQLDCTHL

SEQ ID NO: 297 - protein, 25 aa, Human immunodeficiency virus
QLKGEAIHGQVDCSPGVWQLDCTHL

SEQ ID NO: 298 - protein, 9 aa, Human immunodeficiency virus
GQVDCSPGI

SEQ ID NO: 299 - protein, 9 aa, Human immunodeficiency virus
GQVDCSPGV

SEQ ID NO: 300 - protein, 25 aa, Human immunodeficiency virus
QVDCSPGIWQLDCTHLEGKIILVAV

SEQ ID NO: 301 - protein, 25 aa, Human immunodeficiency virus
QVDCSPGVWQLDCTHLEGKVILVAV

SEQ ID NO: 302 - protein, 9 aa, Human immunodeficiency virus
WQLDCTHLE

SEQ ID NO: 303 - protein, 25 aa, Human immunodeficiency virus
SNFTSTTVKAACWWAGIKQEFGIPY

SEQ ID NO: 304 - protein, 25 aa, Human immunodeficiency virus
SNFTSTAVKAACWWAGVKQEFGIPY

SEQ ID NO: 305 - protein, 80 aa, Human immunodeficiency virus
TVKAACWWAGIKQEFGIPYNPQSQGVVESMNKELKKIIGQVRDQAEHLKTAVQMAVFIHNFKRKGGIGGYSAGERIVDII

SEQ ID NO: 306 - protein, 80 aa, Human immunodeficiency virus
AVKAACWWAGVKQEFGIPYHPQSQGVVESMNNELKKIIGQIRDQAEQLKTAVQMAVLIHNFKRKGGIGEYSAGERIIDII

SEQ ID NO: 307 - protein, 81 aa, Human immunodeficiency virus
TVKAACWWAGIKQEFGIPYNPQSQGVVESMNKELKKIIGQVRDQAEHLKTAVQMAVFIHNFKRKGGIGGYSAGERIVDIIA

SEQ ID NO: 308 - protein, 81 aa, Human immunodeficiency virus
AVKAACWWAGVKQEFGIPYHPQSQGVVESMNNELKKIIGQIRDQAEQLKTAVQMAVLIHNFKRKGGIGEYSAGERIIDIIA

SEQ ID NO: 309 - protein, 164 aa, Human immunodeficiency virus
TVKAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRDPLWKGPAKLLWKGEGAVVIQDNSDIKVVPRRKAKIIRDYGKQMAGDDCVASRQDED

SEQ ID NO: 310 - protein, 164 aa, Human immunodeficiency virus
AVKAACWWAGVKQEFGIPYNTQSQGVVESMNNELKKIIGQIRDQAEHLKTAVQMAVLIHNFKRKGGIGEYSAGERIIDIIATDIQTRELQKQITKLQNFRVYYRDNRDPLWKGPARLLWKGEGAVVIQDNSEIKVVPRRKVKIIRDYGKRMAGDDCVAGRQDED

SEQ ID NO: 311 - protein, 9 aa, Human immunodeficiency virus
KAACWWAGI

SEQ ID NO: 312 - protein, 9 aa, Human immunodeficiency virus
KAACWWAGV

SEQ ID NO: 313 - protein, 9 aa, Human immunodeficiency virus
DIIATDIQT

SEQ ID NO: 314 - protein, 9 aa, Human immunodeficiency virus
DIIASDIQT

SEQ ID NO: 315 - protein, 9 aa, Human immunodeficiency virus
DIQTKELQK

SEQ ID NO: 316 - protein, 9 aa, Human immunodeficiency virus
DIQTRELQK

SEQ ID NO: 317 - protein, 9 aa, Human immunodeficiency virus
QTKELQKQI

SEQ ID NO: 318 - protein, 9 aa, Human immunodeficiency virus
QTRELQKQI

SEQ ID NO: 319 - protein, 73 aa, Human immunodeficiency virus
AITKIQNFRVYYRDSRDPLWKGPAKLLWKGEGAVVIQDNSDIKVVPRRKAKIIRDYGKQMAGDDCVASRQDED

SEQ ID NO: 320 - protein, 73 aa, Human immunodeficiency virus
AITKLQNFRVYYRDNRDPLWKGPARLLWKGEGAVVIQDNSEIKVVPRRKVKIIRDYGKRMAGDDCVAGRQDED

SEQ ID NO: 321 - protein, 72 aa, Human immunodeficiency virus
ITKIQNFRVYYRDSRDPLWKGPAKLLWKGEGAVVIQDNSDIKVVPRRKAKIIRDYGKQMAGDDCVASRQDED

SEQ ID NO: 322 - protein, 72 aa, Human immunodeficiency virus
ITKLQNFRVYYRDNRDPLWKGPARLLWKGEGAVVIQDNSEIKVVPRRKVKIIRDYGKRMAGDDCVAGRQDED

SEQ ID NO: 323 - protein, 25 aa, Human immunodeficiency virus
VYYRDSRDPLWKGPAKLLWKGEGAV

SEQ ID NO: 324 - protein, 25 aa, Human immunodeficiency virus
VYYRDNRDPLWKGPARLLWKGEGAV

SEQ ID NO: 325 - protein, 25 aa, Human immunodeficiency virus
DPLWKGPAKLLWKGEGAVVIQDNSD

SEQ ID NO: 326 - protein, 25 aa, Human immunodeficiency virus
DPLWKGPARLLWKGEGAVVIQDNSE

SEQ ID NO: 327 - protein, 9 aa, Human immunodeficiency virus
PLWKGPAKL

SEQ ID NO: 328 - protein, 9 aa, Human immunodeficiency virus
PLWKGPARL

SEQ ID NO: 329 - protein, 25 aa, Human immunodeficiency virus
PLWKGPAKLLWKGEGAVVIQDNSDI

SEQ ID NO: 330 - protein, 25 aa, Human immunodeficiency virus
PLWKGPARLLWKGEGAVVIQDNSEI

SEQ ID NO: 331 - protein, 9 aa, Human immunodeficiency virus
KLLWKGEGA

SEQ ID NO: 332 - protein, 9 aa, Human immunodeficiency virus
RLLWKGEGA

SEQ ID NO: 333 - protein, 9 aa, Human immunodeficiency virus
LLWKGEGAV

SEQ ID NO: 334 - protein, 24 aa, Human immunodeficiency virus
AKIIRDYGKQMAGDDCVASRQDED

SEQ ID NO: 335 - protein, 24 aa, Human immunodeficiency virus
VKIIRDYGKRMAGDDCVAGRQDED

SEQ ID NO: 336 - protein, 9 aa, Human immunodeficiency virus
KQMAGDDCV

SEQ ID NO: 337 - protein, 9 aa, Human immunodeficiency virus
KRMAGDDCV

SEQ ID NO: 338 - protein, 25 aa, Human immunodeficiency virus
YLRDQQLLGLWGCSGKLICPTAVPW

SEQ ID NO: 339 - protein, 26 aa, Human immunodeficiency virus
PVGEIYKRWIILGLNKIVRMYSPTSI

SEQ ID NO: 340 - protein, 26 aa, Human immunodeficiency virus
PVGDIYKRWIIMGLNKIVRMYSPVSI

SEQ ID NO: 341 - protein, 26 aa, Human immunodeficiency virus
GPKEPFRDYVDRFYKTLRAEQASQEV

SEQ ID NO: 342 - protein, 26 aa, Human immunodeficiency virus
GPKEPFRDYVDRFYRTLRAEQASQDV

SEQ ID NO: 343 - protein, 25 aa, Human immunodeficiency virus
FRKQNPDIVIYQYVDDLYVGSDLEI

SEQ ID NO: 344 - protein, 25 aa, Human immunodeficiency virus
KQNPDIVIYQYVDDLYVGSDLEIEQ

SEQ ID NO: 345 - protein, 771 aa, synthetic construct
  REGION 1..771: note = source = /note="Description of Artificial Sequence: Synthetic polypeptide"
LKHIVWASRELERFAVNPGLLETAAAVSQNYPIVQNAAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQAAALPGRWKPKMIGGIGGFIKVRQYDQAAAGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSAAAWGFTTPDKKHQKEPPFLWMGYELHPDKWTVQPIAAAVAKEIVASCDKCQLKGEAMHGQVDCSPGIWQLDCTHLEGKIILVAVHVASGYIEAEVIPAETGQETAYFLLKLAGRWPVKTAAATVKAACWWAGIKQEFGIPYNPQSQGVVESMNKELKKIIGQVRDQAEHLKTAVQMAVFIHNFKRKGGIGGYSAGERIVDIIAAAAITKIQNFRVYYRDSRDPLWKGPAKLLWKGEGAVVIQDNSDIKVVPRRKAKIIRDYGKQMAGDDCVASRQDEDAAAEEVGFPVKPQVPLRPMTFKGALDLSHFLREKGGLEG

SEQ ID NO: 346 - protein, 771 aa, synthetic construct
  REGION 1..771: note = source = /note="Description of Artificial Sequence: Synthetic polypeptide"
LKHLVWASRELERFALNPGLLETAAAVSQNFPIVQNAAALSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQAAALPGKWKPKMIGGIGGFIKVKQYDQAAAGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSAAAWGLTTPDKKHQKDPPFLWMGYELHPDRWTVQPIAAAVAKEIVACCDKCQLKGEAIHGQVDCSPGVWQLDCTHLEGKVILVAVHVASGYMEAEVIPTETGQETAYFILKLAGRWPVTTAAAAVKAACWWAGVKQEFGIPYHPQSQGVVESMNNELKKIIGQIRDQAEQLKTAVQMAVLIHNFKRKGGIGEYSAGERIIDIIAAAAITKLQNFRVYYRDNRDPLWKGPARLLWKGEGAVVIQDNSEIKVVPRRKVKIIRDYGKRMAGDDCVAGRQDEDAAAEEVGFPVRPQVPLRPMTYKGALDLSHFLKEKGGLEG

SEQ ID NO: 347 - protein, 800 aa, synthetic construct
  REGION 1..800: note = source = /note="Description of Artificial Sequence: Synthetic polypeptide"
LKHIVWASRELERFAVNPGLLETVSQNYPIVQNISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQRAKRAPVKQTLNFDLLKLAGDVESNPGPLPGRWKPKMIGGIGGFIKVRQYDQGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSWGFTTPDKKHQKEPPFLWMGYELHPDKWTVQPIVAKEIVASCDKCQLKGEAMHGQVDCSPGIWQLDCTHLEGKIILVAVHVASGYIEAEVIPAETGQETAYFLLKLAGRWPVKTTVKAACWWAGIKQEFGIPYNPQSQGVVESMNKELKKIIGQVRDQAEHLKTAVQMAVFIHNFKRKGGIGGYSAGERIVDIIAITKIQNFRVYYRDSRDPLWKGPAKLLWKGEGAVVIQDNSDIKVVPRRKAKIIRDYGKQMAGDDCVASRQDEDRAKRAPVKQTLNFDLLKLAGDVESNPGPEEVGFPVKPQVPLRPMTFKGALDLSHFLREKGGLEG

SEQ ID NO: 348 - protein, 800 aa, synthetic construct
  REGION 1..800: note = source = /note="Description of Artificial Sequence: Synthetic polypeptide"
LKHLVWASRELERFALNPGLLETVSQNFPIVQNLSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQRAKRAPVKQTLNFDLLKLAGDVESNPGPLPGKWKPKMIGGIGGFIKVKQYDQGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSWGLTTPDKKHQKDPPFLWMGYELHPDRWTVQPIVAKEIVACCDKCQLKGEAIHGQVDCSPGVWQLDCTHLEGKVILVAVHVASGYMEAEVIPTETGQETAYFILKLAGRWPVTTAVKAACWWAGVKQEFGIPYHPQSQGVVESMNNELKKIIGQIRDQAEQLKTAVQMAVLIHNFKRKGGIGEYSAGERIIDIIAITKLQNFRVYYRDNRDPLWKGPARLLWKGEGAVVIQDNSEIKVVPRRKVKIIRDYGKRMAGDDCVAGRQDEDRAKRAPVKQTLNFDLLKLAGDVESNPGPEEVGFPVRPQVPLRPMTYKGALDLSHFLKEKGGLEG

SEQ ID NO: 349 - protein, 744 aa, synthetic construct
  REGION 1..744: note = source = /note="Description of Artificial Sequence: Synthetic polypeptide"
LKHIVWASRELERFAVNPGLLETVSQNYPIVQNISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQLPGRWKPKMIGGIGGFIKVRQYDQGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSWGFTTPDKKHQKEPPFLWMGYELHPDKWTVQPIVAKEIVASCDKCQLKGEAMHGQVDCSPGIWQLDCTHLEGKIILVAVHVASGYIEAEVIPAETGQETAYFLLKLAGRWPVKTTVKAACWWAGIKQEFGIPYNPQSQGVVESMNKELKKIIGQVRDQAEHLKTAVQMAVFIHNFKRKGGIGGYSAGERIVDIIAITKIQNFRVYYRDSRDPLWKGPAKLLWKGEGAVVIQDNSDIKVVPRRKAKIIRDYGKQMAGDDCVASRQDEDEEVGFPVKPQVPLRPMTFKGALDLSHFLREKGGLEG

SEQ ID NO: 350 - protein, 744 aa, synthetic construct
  REGION 1..744: note = source = /note="Description of Artificial Sequence: Synthetic polypeptide"
LKHLVWASRELERFALNPGLLETVSQNFPIVQNLSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQLPGKWKPKMIGGIGGFIKVKQYDQGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSWGLTTPDKKHQKDPPFLWMGYELHPDRWTVQPIVAKEIVACCDKCQLKGEAIHGQVDCSPGVWQLDCTHLEGKVILVAVHVASGYMEAEVIPTETGQETAYFILKLAGRWPVTTAVKAACWWAGVKQEFGIPYHPQSQGVVESMNNELKKIIGQIRDQAEQLKTAVQMAVLIHNFKRKGGIGEYSAGERIIDIIAITKLQNFRVYYRDNRDPLWKGPARLLWKGEGAVVIQDNSEIKVVPRRKVKIIRDYGKRMAGDDCVAGRQDEDEEVGFPVRPQVPLRPMTYKGALDLSHFLKEKGGLEG

SEQ ID NO: 351 - protein, 346 aa, synthetic construct
  REGION 1..346: note = source = /note="Description of Artificial Sequence: Synthetic polypeptide"
MGARASVLSGGELDRWEKIRLRPGGKKKYRLKHIVWASRELERFAVNPGLLETAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQEEVGFPVKPQVPLRPMTFKGALDLSHFLREKGGLEGTQGFFPDWQNYTPEPGIRFPLTFGWCFKLVPL

SEQ ID NO: 352 - protein, 344 aa, synthetic construct
  REGION 1..344: note = source = /note="Description of Artificial Sequence: Synthetic polypeptide"
LSPRTLNAWVKVIEEKAFSPEVIPMFTALSEGATPHDLNTMLNTIGGHQAAMQMLKDTINEEAAEWDRVHPVHAGPVAPGQMRDPRGSDIAGSTSTLQEQIAWMTNNPPIPVGDIYKRWIIMGLNKIVRMYSPVSILDIKQGPKEPFRDYVDRFYRTLRAEQASQDVKNWMTETLLVQNSNPDCKTILKALGPGATLEEMMSACQGVGGPSHKARVLAEAMCQMGARASILSGGKLDKWEKIRLRPGGRKKYKLKHIVWASRELERFAVNPGLLETEEVGFPVRPQVPLRPMTYKGALDLSHFLKEKGGLEGTQGYFPDWQNYTPGPGTRYPLTFGWCFKLVPV

SEQ ID NO: 353 - protein, 386 aa, synthetic construct
  REGION 1..386: note = source = /note="Description of Artificial Sequence: Synthetic polypeptide"
MWLQSLLLLGTVACSISVMGARASVLSGGELDRWEKIRLRPGGKKKYRLKHIVWASRELERFAVNPGLLETLKHIVWASRELERFAVNPGLLETAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQEEVGFPVKPQVPLRPMTFKGALDLSHFLREKGGLEGTQGFFPDQNYTPEPGIRFPLTFGWCFKLVPL

SEQ ID NO: 354 - protein, 391 aa, synthetic construct
  REGION 1..391: note = source = /note="Description of Artificial Sequence: Synthetic polypeptide"
MDAMKRGLCCVLLLCGAVFVSARMGARASVLSGGELDRWEKIRLRPGGKKKYRLKHIVWASRELERFAVNPGLLETLKHIVWASRELERFAVNPGLLETAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQEEVGFPVKPQVPLRPMTFKGALDLSHFLREKGGLEGTQGFFPDQNYTPEPGIRFPLTFGWCFKLVPL

SEQ ID NO: 355 - protein, 475 aa, synthetic construct
  REGION 1..475: note = source = /note="Description of Artificial Sequence: Synthetic polypeptide"
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLASAGAMGARASVLSGGELDRWEKIRLRPGGKKKYRLKHIVWASRELERFAVNPGLLETLKHIVWASRELERFAVNPGLLETAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQEEVGFPVKPQVPLRPMTFKGALDLSHFLREKGGLEGTQGFFPDQNYTPEPGIRFPLTFGWCFKLVPL

SEQ ID NO: 356 - protein, 399 aa, synthetic construct
  REGION 1..399: note = source = /note="Description of Artificial Sequence: Synthetic polypeptide"
MRKAAVSHWQQQSYLDSGIHSGATTTAPSLSMGARASVLSGGELDRWEKIRLRPGGKKKYRLKHIVWASRELERFAVNPGLLETLKHIVWASRELERFAVNPGLLETAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQEEVGFPVKPQVPLRPMTFKGALDLSHFLREKGGLEGTQGFFPDQNYTPEPGIRFPLTFGWCFKLVPL

SEQ ID NO: 357 - protein, 854 aa, synthetic construct
  REGION 1..854: note = source = /note="Description of Artificial Sequence: Synthetic polypeptide"
GTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTTVKAACWWAGIKQEFGIPYNPQSQGVVESMNKELKKIIGQVRDQAEHLKTAVQMAVFIHNFKRKGGIGGYSAGERIVDIINVSTVQCTHGIRPVVSTQLLLNGSLAEEKRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTVAKEIVASCDKCQLKGEAMHGQVDCSPGIWQLDCTHLEGKIILVAVHVASGYIEAEVIPAETGQETAYFLLKLAGRWPVKTLWVTVYYGVPVWKEAAFPQITLWQRPLVTIKIGGQLKEALLDTGADDTVLEEMNLPGRWKPKMIGGIGGFIKVRQYDQAAAAHNVWATHACVPTDPNPQEAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVIVIYQYMDDLYVGSDLEIGQHRMRDNWRSELYKYKVV

SEQ ID NO: 358 - protein, 849 aa, synthetic construct
  REGION 1..849: note = source = /note="Description of Artificial Sequence: Synthetic polypeptide"
AVKAACWWAGVKQEFGIPYHPQSQGVVESMNNELKKIIGQIRDQAEQLKTAVQMAVLIHNFKRKGGIGEYSAGERIIDIIRRRVVQREKRAIGLGAVFLGFLGTAGSTMGAASMTLTVQARLLLSGIVQQQSNLLRAIEAQQHMLQLTVWGIKQLQARILAVERYLRDQQLLGIWGCSGRLICTTVAKEIVACCDKCQLKGEAIHGQVDCSPGVWQLDCTHLEGKVILVAVHVASGYMEAEVIPTETGQETAYFILKLAGRWPVTTNISTVQCTHGIKPVVSTQLLLNGSLAEKWGLTTPDKKHQKDPPFLWMGYELHPDRWTVQPIELPEKESWTVNDIQKLIGKLNWASQIYAGIKVIVIYQYVDDLYVGSDLEIEQHRPKFRLPIQKETWDTWWTDYWQATWIPEWEFTNTPPLVKLWYQLETEPIAGVETFYVDGASNRETKLPQITLWQRPIVTIKIGGQIKEALLDTGADDTVLEDMNLPGKWKPKMIGGIGGFIKVKQYDQAALWVTIYYGVPVWKDVHNIWATHACVPTDPSPQEAITKLQNFRVYYRDNRDPLWKGPARLLWKGEGAVVIQDNSEIKVVPRRKVKIIRDYGKRMAGDDCVAGRQDEDGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMTKEKIYLAWVPAHKGIGGNEQIDKLVSMKDNWRSELYRYKVV

SEQ ID NO: 359 - protein, 486 aa, synthetic construct
  REGION 1..486: note = source = /note="Description of Artificial Sequence: Synthetic polypeptide"
GTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTTVKAACWWAGIKQEFGIPYNPQSQGVVESMNKELKKIIGQVRDQAEHLKTAVQMAVFIHNFKRKGGIGGYSAGERIVDIINVSTVQCTHGIRPVVSTQLLLNGSLAEEKRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTVAKEIVASCDKCQLKGEAMHGQVDCSPGIWQLDCTHLEGKIILVAVHVASGYIEAEVIPAETGQETAYFLLKLAGRWPVKT

SEQ ID NO: 360 - protein, 368 aa, synthetic construct
  REGION 1..368: note = source = /note="Description of Artificial Sequence: Synthetic polypeptide"
LWVTVYYGVPVWKEAAFPQITLWQRPLVTIKIGGQLKEALLDTGADDTVLEEMNLPGRWKPKMIGGIGGFIKVRQYDQAAAAHNVWATHACVPTDPNPQEAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVIVIYQYMDDLYVGSDLEIGQHRMRDNWRSELYKYKVV

SEQ ID NO: 361 - protein, 510 aa, synthetic construct
  REGION 1..510: note = source = /note="Description of Artificial Sequence: Synthetic polypeptide"
AVKAACWWAGVKQEFGIPYHPQSQGVVESMNNELKKIIGQIRDQAEQLKTAVQMAVLIHNFKRKGGIGEYSAGERIIDIIRRRVVQREKRAIGLGAVFLGFLGTAGSTMGAASMTLTVQARLLLSGIVQQQSNLLRAIEAQQHMLQLTVWGIKQLQARILAVERYLRDQQLLGIWGCSGRLICTTVAKEIVACCDKCQLKGEAIHGQVDCSPGVWQLDCTHLEGKVILVAVHVASGYMEAEVIPTETGQETAYFILKLAGRWPVTTNISTVQCTHGIKPVVSTQLLLNGSLAEKWGLTTPDKKHQKDPPFLWMGYELHPDRWTVQPIELPEKESWTVNDIQKLIGKLNWASQIYAGIKVIVIYQYVDDLYVGSDLEIEQHRPKFRLPIQKETWDTWWTDYWQATWIPEWEFTNTPPLVKLWYQLETEPIAGVETFYVDGASNRETKLPQITLWQRPIVTIKIGGQIKEALLDTGADDTVLEDMNLPGKWKPKMIGGIGGFIKVKQYDQAA

SEQ ID NO: 362 - protein, 339 aa, synthetic construct
  REGION 1..339: note = source = /note="Description of Artificial Sequence: Synthetic polypeptide"
LWVTIYYGVPVWKDVHNIWATHACVPTDPSPQEAITKLQNFRVYYRDNRDPLWKGPARLLWKGEGAVVIQDNSEIKVVPRRKVKIIRDYGKRMAGDDCVAGRQDEDGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMTKEKIYLAWVPAHKGIGGNEQIDKLVSMKDNWRSELYRYKVV

SEQ ID NO: 363 - protein, 872 aa, synthetic construct
  REGION 1..872: note = source = /note="Description of Artificial Sequence: Synthetic polypeptide"
MWLQSLLLLGTVACSISVGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTTVKAACWWAGIKQEFGIPYNPQSQGVVESMNKELKKIIGQVRDQAEHLKTAVQMAVFIHNFKRKGGIGGYSAGERIVDIINVSTVQCTHGIRPVVSTQLLLNGSLAEEKRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTVAKEIVASCDKCQLKGEAMHGQVDCSPGIWQLDCTHLEGKIILVAVHVASGYIEAEVIPAETGQETAYFLLKLAGRWPVKTLWVTVYYGVPVWKEAAFPQITLWQRPLVTIKIGGQLKEALLDTGADDTVLEEMNLPGRWKPKMIGGIGGFIKVRQYDQAAAAHNVWATHACVPTDPNPQEAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVIVIYQYMDDLYVGSDLEIGQHRMRDNWRSELYKYKVV

SEQ ID NO: 364 - protein, 877 aa, synthetic construct
  REGION 1..877: note = source = /note="Description of Artificial Sequence: Synthetic polypeptide"
MDAMKRGLCCVLLLCGAVFVSARGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTTVKAACWWAGIKQEFGIPYNPQSQGVVESMNKELKKIIGQVRDQAEHLKTAVQMAVFIHNFKRKGGIGGYSAGERIVDIINVSTVQCTHGIRPVVSTQLLLNGSLAEEKRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTVAKEIVASCDKCQLKGEAMHGQVDCSPGIWQLDCTHLEGKIILVAVHVASGYIEAEVIPAETGQETAYFLLKLAGRWPVKTLWVTVYYGVPVWKEAAFPQITLWQRPLVTIKIGGQLKEALLDTGADDTVLEEMNLPGRWKPKMIGGIGGFIKVRQYDQAAAAHNVWATHACVPTDPNPQEAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVIVIYQYMDDLYVGSDLEIGQHRMRDNWRSELYKYKVV

SEQ ID NO: 365 - protein, 961 aa, synthetic construct
  REGION 1..961: note = source = /note="Description of Artificial Sequence: Synthetic polypeptide"
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLASAGAGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTTVKAACWWAGIKQEFGIPYNPQSQGVVESMNKELKKIIGQVRDQAEHLKTAVQMAVFIHNFKRKGGIGGYSAGERIVDIINVSTVQCTHGIRPVVSTQLLLNGSLAEEKRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTVAKEIVASCDKCQLKGEAMHGQVDCSPGIWQLDCTHLEGKIILVAVHVASGYIEAEVIPAETGQETAYFLLKLAGRWPVKTLWVTVYYGVPVWKEAAFPQITLWQRPLVTIKIGGQLKEALLDTGADDTVLEEMNLPGRWKPKMIGGIGGFIKVRQYDQAAAAHNVWATHACVPTDPNPQEAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVIVIYQYMDDLYVGSDLEIGQHRMRDNWRSELYKYKVV

SEQ ID NO: 366 - protein, 885 aa, synthetic construct
  REGION 1..885: note = source = /note="Description of Artificial Sequence: Synthetic polypeptide"
MRKAAVSHWQQQSYLDSGIHSGATTTAPSLSGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTTVKAACWWAGIKQEFGIPYNPQSQGVVESMNKELKKIIGQVRDQAEHLKTAVQMAVFIHNFKRKGGIGGYSAGERIVDIINVSTVQCTHGIRPVVSTQLLLNGSLAEEKRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTVAKEIVASCDKCQLKGEAMHGQVDCSPGIWQLDCTHLEGKIILVAVHVASGYIEAEVIPAETGQETAYFLLKLAGRWPVKTLWVTVYYGVPVWKEAAFPQITLWQRPLVTIKIGGQLKEALLDTGADDTVLEEMNLPGRWKPKMIGGIGGFIKVRQYDQAAAAHNVWATHACVPTDPNPQEAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVIVIYQYMDDLYVGSDLEIGQHRMRDNWRSELYKYKVV

SEQ ID NO: 367 - protein, 363 aa, synthetic construct
  REGION 1..363: note = source = /note="Description of Artificial Sequence: Synthetic polypeptide"
YQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLRAEQASQEVDLNTMLNTVKLTPLCVTLYQYMDDLYVVIYQYMDDL

SEQ ID NO: 368 - protein, 381 aa, synthetic construct
  REGION 1..381: note = source = /note="Description of Artificial Sequence: Synthetic polypeptide"
MWLQSLLLLGTVACSISVYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLRAEQASQEVDLNTMLNTVKLTPLCVTLYQYMDDLYVVIYQYMDDL

SEQ ID NO: 369 - protein, 386 aa, synthetic construct
  REGION 1..386: note = source = /note="Description of Artificial Sequence: Synthetic polypeptide"
MDAMKRGLCCVLLLCGAVFVSARYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLRAEQASQEVDLNTMLNTVKLTPLCVTLYQYMDDLYVVIYQYMDDL

SEQ ID NO: 370 - protein, 470 aa, synthetic construct
  REGION 1..470: note = source = /note="Description of Artificial Sequence: Synthetic polypeptide"
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLASAGAYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLRAEQASQEVDLNTMLNTVKLTPLCVTLYQYMDDLYVVIYQYMDDL

SEQ ID NO: 371 - protein, 394 aa, synthetic construct
  REGION 1..394: note = source = /note="Description of Artificial Sequence: Synthetic polypeptide"
MRKAAVSHWQQQSYLDSGIHSGATTTAPSLSYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLRAEQASQEVDLNTMLNTVKLTPLCVTLYQYMDDLYVVIYQYMDDL

SEQ ID NO: 372 - protein, 928 aa, synthetic construct
  REGION 1..928: note = source = /note="Description of Artificial Sequence: Synthetic polypeptide"
MAPRSARRPLLLLLLLLLLGLMHCASAAMFMVKNGNGTACIMANFSAAFSVNYDTKSGPKNMTLDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTLLPDARDPAFKAANGSLRALQATVGNSYKCNAEEHVRVTKAFSVNIFKVWVQAFKVEGGQFGSVEECLLDENSLEDIRTLNAWVKVREKRDLNTMLNTVREKRWIILGLNKIREKRYVDRFYKTLREKRATLEEMMTAREKREMMTACQGVREKRTLNFPISPIREKRYQYNVLPQGREKRVIYQYMDDLREKRYQYMDDLYVREKRYMDDLYVGSREKRFLWMGYELHREKRELHPDKWTVREKRWTVNDIQKLREKRTVNDIQKLVREKRKLVGKLNWAREKRWQATWIPEWREKRIVTDSQYALREKRGQVDCSPGIREKRWQLDCTHLEREKRKAACWWAGIREKRPLWKGPAKLREKRKLLWKGEGAREKRLLWKGEGAVREKRKQMAGDDCVREKRVTVYYGVPVREKRNVWATHACVREKRKLTPLCVTLREKRSTVQCTHGIREKRGIWGCSGKLREKRLTFGWCFKLREKRASRELERFAVNPGLLREKRWIILGLNKIVRMYSPTSIREKRYVDRFYKTLRAEQASQEVREKRRQANFLGKIWPSHKGRREKRYKAAVDLSHFLREKGGLEGGSEFTLIPIAVGGALAGLVIVLIAYLVGRKRSHAGYQTI

SEQ ID NO: 373 - protein, 847 aa, synthetic construct
  REGION 1..847: note = source = /note="Description of Artificial Sequence: Synthetic polypeptide"
ASRELERFAVNPGLLQLKGEAMHGQVDCSPGIWQLDCTHLCSATEKLWVTVYYGVPVWKEATTTLPVGEIYKRWIILGLNKIVRMYSPTSINNETPGIRYQYNVLPQGWKGSPAIFAAPLWKGPAKLLWKGEGAVVIQDNSDIAAIVLPEKDSWTVNDIQKLVGKLNWASVYYRDSRDPLWKGPAKLLWKGEGAVSNFTSTTVKAACWWAGIKQEFGIPYVLPEKDSWTVNDIQKLVGKLNWASQMVHQAISPRTLNAWVKVVEEKAFSPAAWETWWTEYWQATWIPEWEFVNTPPLQDSGLEVNIVTDSQYALGIIQAQPDSWTVNDIQKLVGKLNWASQIYPGIKNPDIVIYQYMDDLYVGSDLEIGQHRAADPLWKGPAKLLWKGEGAVVIQDNSDRQANFLGKIWPSHKGRAAPPFLWMGYELHPDKWTVQPIVLPEKFRKQNPDIVIYQYMDDLYVGSDLEINLLTQIGCTLNFPISPIETVPVKLKGPKEPFRDYVDRFYKTLRAEQASQEVYLKDQQLLGIWGCSGKLICTTAVPWAAAKKHQKEPPFLWMGYELHPDKWTVQPGTGPCTNVSTVQCTHGIRPVVSTQLKQNPDIVIYQYMDDLYVGSDLEIGQLGPAATLEEMMTACQGVGGPGHKARDQSLKPCVKLTPLCVTLNCTDLRNTGPGIRYPLLTFGWCFKLPVEPEKVEAKIIRDYGKQMAGDDCVASRQDEDILKALGPAATLEEMMTACQGVGGPGYKAAVDLSHFLREKGGLEGAAYAAKAYDTEVHNVWATHACVPTDPNPQEAAALSEGATPQDLNTMLNTVGGHQAAMQQVDCSPGIWQLDCTHLEGKIILVAV

SEQ ID NO: 374 - protein, 865 aa, synthetic construct
  REGION 1..865: note = source = /note="Description of Artificial Sequence: Synthetic polypeptide"
MWLQSLLLLGTVACSISVASRELERFAVNPGLLQLKGEAMHGQVDCSPGIWQLDCTHLCSATEKLWVTVYYGVPVWKEATTTLPVGEIYKRWIILGLNKIVRMYSPTSINNETPGIRYQYNVLPQGWKGSPAIFAAPLWKGPAKLLWKGEGAVVIQDNSDIAAIVLPEKDSWTVNDIQKLVGKLNWASVYYRDSRDPLWKGPAKLLWKGEGAVSNFTSTTVKAACWWAGIKQEFGIPYVLPEKDSWTVNDIQKLVGKLNWASQMVHQAISPRTLNAWVKVVEEKAFSPAAWETWWTEYWQATWIPEWEFVNTPPLQDSGLEVNIVTDSQYALGIIQAQPDSWTVNDIQKLVGKLNWASQIYPGIKNPDIVIYQYMDDLYVGSDLEIGQHRAADPLWKGPAKLLWKGEGAVVIQDNSDRQANFLGKIWPSHKGRAAPPFLWMGYELHPDKWTVQPIVLPEKFRKQNPDIVIYQYMDDLYVGSDLEINLLTQIGCTLNFPISPIETVPVKLKGPKEPFRDYVDRFYKTLRAEQASQEVYLKDQQLLGIWGCSGKLICTTAVPWAAAKKHQKEPPFLWMGYELHPDKWTVQPGTGPCTNVSTVQCTHGIRPVVSTQLKQNPDIVIYQYMDDLYVGSDLEIGQLGPAATLEEMMTACQGVGGPGHKARDQSLKPCVKLTPLCVTLNCTDLRNTGPGIRYPLLTFGWCFKLPVEPEKVEAKIIRDYGKQMAGDDCVASRQDEDILKALGPAATLEEMMTACQGVGGPGYKAAVDLSHFLREKGGLEGAAYAAKAYDTEVHNVWATHACVPTDPNPQEAAALSEGATPQDLNTMLNTVGGHQAAMQQVDCSPGIWQLDCTHLEGKIILVAV

SEQ ID NO: 375 - protein, 870 aa, synthetic construct
  REGION 1..870: note = source = /note="Description of Artificial Sequence: Synthetic polypeptide"
MDAMKRGLCCVLLLCGAVFVSARASRELERFAVNPGLLQLKGEAMHGQVDCSPGIWQLDCTHLCSATEKLWVTVYYGVPVWKEATTTLPVGEIYKRWIILGLNKIVRMYSPTSINNETPGIRYQYNVLPQGWKGSPAIFAAPLWKGPAKLLWKGEGAVVIQDNSDIAAIVLPEKDSWTVNDIQKLVGKLNWASVYYRDSRDPLWKGPAKLLWKGEGAVSNFTSTTVKAACWWAGIKQEFGIPYVLPEKDSWTVNDIQKLVGKLNWASQMVHQAISPRTLNAWVKVVEEKAFSPAAWETWWTEYWQATWIPEWEFVNTPPLQDSGLEVNIVTDSQYALGIIQAQPDSWTVNDIQKLVGKLNWASQIYPGIKNPDIVIYQYMDDLYVGSDLEIGQHRAADPLWKGPAKLLWKGEGAVVIQDNSDRQANFLGKIWPSHKGRAAPPFLWMGYELHPDKWTVQPIVLPEKFRKQNPDIVIYQYMDDLYVGSDLEINLLTQIGCTLNFPISPIETVPVKLKGPKEPFRDYVDRFYKTLRAEQASQEVYLKDQQLLGIWGCSGKLICTTAVPWAAAKKHQKEPPFLWMGYELHPDKWTVQPGTGPCTNVSTVQCTHGIRPVVSTQLKQNPDIVIYQYMDDLYVGSDLEIGQLGPAATLEEMMTACQGVGGPGHKARDQSLKPCVKLTPLCVTLNCTDLRNTGPGIRYPLLTFGWCFKLPVEPEKVEAKIIRDYGKQMAGDDCVASRQDEDILKALGPAATLEEMMTACQGVGGPGYKAAVDLSHFLREKGGLEGAAYAAKAYDTEVHNVWATHACVPTDPNPQEAAALSEGATPQDLNTMLNTVGGHQAAMQQVDCSPGIWQLDCTHLEGKIILVAV

SEQ ID NO: 376 - protein, 954 aa, synthetic construct
  REGION 1..954: note = source = /note="Description of Artificial Sequence: Synthetic polypeptide"
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLASAGAASRELERFAVNPGLLQLKGEAMHGQVDCSPGIWQLDCTHLCSATEKLWVTVYYGVPVWKEATTTLPVGEIYKRWIILGLNKIVRMYSPTSINNETPGIRYQYNVLPQGWKGSPAIFAAPLWKGPAKLLWKGEGAVVIQDNSDIAAIVLPEKDSWTVNDIQKLVGKLNWASVYYRDSRDPLWKGPAKLLWKGEGAVSNFTSTTVKAACWWAGIKQEFGIPYVLPEKDSWTVNDIQKLVGKLNWASQMVHQAISPRTLNAWVKVVEEKAFSPAAWETWWTEYWQATWIPEWEFVNTPPLQDSGLEVNIVTDSQYALGIIQAQPDSWTVNDIQKLVGKLNWASQIYPGIKNPDIVIYQYMDDLYVGSDLEIGQHRAADPLWKGPAKLLWKGEGAVVIQDNSDRQANFLGKIWPSHKGRAAPPFLWMGYELHPDKWTVQPIVLPEKFRKQNPDIVIYQYMDDLYVGSDLEINLLTQIGCTLNFPISPIETVPVKLKGPKEPFRDYVDRFYKTLRAEQASQEVYLKDQQLLGIWGCSGKLICTTAVPWAAAKKHQKEPPFLWMGYELHPDKWTVQPGTGPCTNVSTVQCTHGIRPVVSTQLKQNPDIVIYQYMDDLYVGSDLEIGQLGPAATLEEMMTACQGVGGPGHKARDQSLKPCVKLTPLCVTLNCTDLRNTGPGIRYPLLTFGWCFKLPVEPEKVEAKIIRDYGKQMAGDDCVASRQDEDILKALGPAATLEEMMTACQGVGGPGYKAAVDLSHFLREKGGLEGAAYAAKAYDTEVHNVWATHACVPTDPNPQEAAALSEGATPQDLNTMLNTVGGHQAAMQQVDCSPGIWQLDCTHLEGKIILVAV

SEQ ID NO: 377 - protein, 878 aa, synthetic construct
  REGION 1..878: note = source = /note="Description of Artificial Sequence: Synthetic polypeptide"
MRKAAVSHWQQQSYLDSGIHSGATTTAPSLSASRELERFAVNPGLLQLKGEAMHGQVDCSPGIWQLDCTHLCSATEKLWVTVYYGVPVWKEATTTLPVGEIYKRWIILGLNKIVRMYSPTSINNETPGIRYQYNVLPQGWKGSPAIFAAPLWKGPAKLLWKGEGAVVIQDNSDIAAIVLPEKDSWTVNDIQKLVGKLNWASVYYRDSRDPLWKGPAKLLWKGEGAVSNFTSTTVKAACWWAGIKQEFGIPYVLPEKDSWTVNDIQKLVGKLNWASQMVHQAISPRTLNAWVKVVEEKAFSPAAWETWWTEYWQATWIPEWEFVNTPPLQDSGLEVNIVTDSQYALGIIQAQPDSWTVNDIQKLVGKLNWASQIYPGIKNPDIVIYQYMDDLYVGSDLEIGQHRAADPLWKGPAKLLWKGEGAVVIQDNSDRQANFLGKIWPSHKGRAAPPFLWMGYELHPDKWTVQPIVLPEKFRKQNPDIVIYQYMDDLYVGSDLEINLLTQIGCTLNFPISPIETVPVKLKGPKEPFRDYVDRFYKTLRAEQASQEVYLKDQQLLGIWGCSGKLICTTAVPWAAAKKHQKEPPFLWMGYELHPDKWTVQPGTGPCTNVSTVQCTHGIRPVVSTQLKQNPDIVIYQYMDDLYVGSDLEIGQLGPAATLEEMMTACQGVGGPGHKARDQSLKPCVKLTPLCVTLNCTDLRNTGPGIRYPLLTFGWCFKLPVEPEKVEAKIIRDYGKQMAGDDCVASRQDEDILKALGPAATLEEMMTACQGVGGPGYKAAVDLSHFLREKGGLEGAAYAAKAYDTEVHNVWATHACVPTDPNPQEAAALSEGATPQDLNTMLNTVGGHQAAMQQVDCSPGIWQLDCTHLEGKIILVAV

SEQ ID NO: 378-380 - skipped (3 entries)

SEQ ID NO: 381 - protein, 4 aa, synthetic construct
  REGION 1..4: note = source = /note="Description of Artificial Sequence: Synthetic peptide"
RAKR

SEQ ID NO: 382 - protein, 4 aa, synthetic construct
  REGION 1..4: note = source = /note="Description of Artificial Sequence: Synthetic peptide"
REKR

SEQ ID NO: 383 - protein, 4 aa, synthetic construct
  REGION 1..4: note = source = /note="Description of Artificial Sequence: Synthetic peptide"
RRKR

SEQ ID NO: 384 - protein, 19 aa, synthetic construct
  REGION 1..19: note = source = /note="Description of Artificial Sequence: Synthetic peptide"
ATNFSLLKQAGDVEENPGP

SEQ ID NO: 385 - protein, 24 aa, synthetic construct
  REGION 1..24: note = source = /note="Description of Artificial Sequence: Synthetic peptide"
APVKQTLNFDLLKLAGDVESNPGP

SEQ ID NO: 386 - protein, 28 aa, synthetic construct
  REGION 1..28: note = source = /note="Description of Artificial Sequence: Synthetic peptide"
RAKRAPVKQTLNFDLLKLAGDVESNPGP

SEQ ID NO: 387 - protein, 20 aa, synthetic construct
  REGION 1..20: note = source = /note="Description of Artificial Sequence: Synthetic peptide"
QCTNYALLKLAGDVESNPGP

SEQ ID NO: 388 - protein, 18 aa, synthetic construct
  REGION 1..18: note = source = /note="Description of Artificial Sequence: Synthetic peptide"
EGRGSLLTCGDVEENPGP

SEQ ID NO: 389 - protein, 7 aa, synthetic construct
  REGION 1..7: note = source = /note="Description of Artificial Sequence: Synthetic peptide"
VHAGPIA

SEQ ID NO: 390 - protein, 7 aa, synthetic construct
  REGION 1..7: note = source = /note="Description of Artificial Sequence: Synthetic peptide"
VHAGPVA

SEQ ID NO: 391 - protein, 5 aa, synthetic construct
  REGION 1..5: note = source = /note="Description of Artificial Sequence: Synthetic peptide"
GALDI

SEQ ID NO: 392 - protein, 5 aa, synthetic construct
  REGION 1..5: note = source = /note="Description of Artificial Sequence: Synthetic peptide"
GALDL

SEQ ID NO: 393 - protein, 18 aa, unidentified
  REGION 1..18: note = source = /note="Description of Unknown: GM-CSF sequence"
MWLQSLLLLGTVACSISV

SEQ ID NO: 394 - protein, 23 aa, unidentified
  REGION 1..23: note = source = /note="Description of Unknown: Tissue-type plasminogen activator (t-PA) sequence"
MDAMKRGLCCVLLLCGAVFVSAR

SEQ ID NO: 395 - protein, 16 aa, unidentified
  REGION 1..16: note = source = /note="Description of Unknown: Invariant chain (CD74) sequence"
MHRRRSRSCREDQKPV

SEQ ID NO: 396 - protein, 17 aa, unidentified
  REGION 1..17: note = source = /note="Description of Unknown: Serum albumin sequence"
KWVTFISLLFLFSSAYS

SEQ ID NO: 397 - protein, 31 aa, unidentified
  REGION 1..31: note = source = /note="Description of Unknown: Beta-catenin sequence"
MRKAAVSHWQQQSYLDSGIHSGATTTAPSLS

SEQ ID NO: 398 - protein, 107 aa, unidentified
  REGION 1..107: note = source = /note="Description of Unknown: MCP-3 sequence"
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLASAGA

SEQ ID NO: 399 - protein, 384 aa, unidentified
  REGION 1..384: note = source = /note="Description of Unknown: Lysosomal-associated membrane glycoprotein-1 (LAMP-1) sequence"
MAPRSARRPLLLLLLLLLLGLMHCASAAMFMVKNGNGTACIMANFSAAFSVNYDTKSGPKNMTLDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTLLPDARDPAFKAANGSLRALQATVGNSYKCNAEEHVRVTKAFSVNIFKVWVQAFKVEGGQFGSVEECLLDENSLEDI

SEQ ID NO: 400 - protein, 76 aa, unidentified [sequence omitted]

SEQ ID NO: 401 - protein, 17 aa, unidentified
  REGION 1..17: note = source = /note="Description of Unknown: Calreticulin (CALR) sequence"
MLLSVPLLLGLLGLAVA

SEQ ID NO: 402 - protein, 16 aa, Vesicular stomatitis virus
MKCLLYLAFLFIGVNC

SEQ ID NO: 403 - protein, 856 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 404 - protein, 500 aa, Human immunodeficiency virus
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 405 - protein, 206 aa, Human immunodeficiency virus
MGGKWSKSSVIGWPTVRERMRRAEPAADRVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKIEEANKGENTSLLHPVSLHGMDDPEREVLEWRFDSRLAFHHVARELHPEYFKNC

SEQ ID NO: 406 - protein, 1003 aa, Human immunodeficiency virus
FFREDLAFLQGKAREFSSEQTRANSPTRRELQVWGRDNNSPSEAGADRQGTVSFNFPQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 407 - protein, 670 aa, synthetic construct
  REGION 1..670: note = source = /note="Description of Artificial Sequence: Synthetic polypeptide"
TVKAACWWAGIKQEFGIPYNPQSQGVVESMNKELKKIIGQVRDQAEHLKTAVQMAVFIHNFKRKGGIGGYSAGERIVDIIAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVIVIYQYMDDLYVGSDLEIGQHRVAKEIVASCDKCQLKGEAMHGQVDCSPGIWQLDCTHLEGKIILVAVHVASGYIEAEVIPAETGQETAYFLLKLAGRWPVKTAAFPQITLWQRPLVTIKIGGQLKEALLDTGADDTVLEEMNLPGRWKPKMIGGIGGFIKVRQYDQGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 408 - protein, 669 aa, synthetic construct
  REGION 1..669: note = source = /note="Description of Artificial Sequence: Synthetic polypeptide"
AVKAACWWAGVKQEFGIPYHPQSQGVVESMNNELKKIIGQIRDQAEQLKTAVQMAVLIHNFKRKGGIGEYSAGERIIDIIVAKEIVACCDKCQLKGEAIHGQVDCSPGVWQLDCTHLEGKVILVAVHVASGYMEAEVIPTETGQETAYFILKLAGRWPVTTWGLTTPDKKHQKDPPFLWMGYELHPDRWTVQPIELPEKESWTVNDIQKLIGKLNWASQIYAGIKVIVIYQYVDDLYVGSDLEIEQHRLPQITLWQRPIVTIKIGGQIKEALLDTGADDTVLEDMNLPGKWKPKMIGGIGGFIKVKQYDQPKFRLPIQKETWDTWWTDYWQATWIPEWEFTNTPPLVKLWYQLETEPIAGVETFYVDGASNRETKAAYAITKLQNFRVYYRDNRDPLWKGPARLLWKGEGAVVIQDNSEIKVVPRRKVKIIRDYGKRMAGDDCVAGRQDEDGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMTKEKIYLAWVPAHKGIGGNEQIDKLVS

SEQ ID NO: 409 - protein, 648 aa, synthetic construct
  REGION 1..648: note = source = /note="Description of Artificial Sequence: Synthetic polypeptide"
TVKAACWWAGIKQEFGIPYNPQSQGVVESMNKELKKIIGQVRDQAEHLKTAVQMAVFIHNFKRKGGIGGYSAGERIVDIIVAKEIVASCDKCQLKGEAMHGQVDCSPGIWQLDCTHLEGKIILVAVHVASGYIEAEVIPAETGQETAYFLLKLAGRWPVKTAITKIQNFRVYYRDSRDPLWKGPAKLLWKGEGAVVIQDNSDIKVVPRRKAKIIRDYGKQMAGDDCVASRQDEDPKFKLPIQKETWETWWTEYWQATWIPEWEFVNTPPLVKLWYQLEKEPIVGAETFYVDGAANRETKAAKEKVYLAWVPAHKGIGGNEQVDKLVSWGFTTPDKKHQKEPPFLWMGYELHPDKWTVQPIVLPEKDSWTVNDIQKLVGKLNWASQIYPGIKVAAFPQITLWQRPLVTIKIGGQLKEALLDTGADDTVLEEMNLPGRWKPKMIGGIGGFIKVRQYDQGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMT

SEQ ID NO: 410 - protein, 650 aa, synthetic construct
  REGION 1..650: note = source = /note="Description of Artificial Sequence: Synthetic polypeptide"
AVKAACWWAGVKQEFGIPYHPQSQGVVESMNNELKKIIGQIRDQAEQLKTAVQMAVLIHNFKRKGGIGEYSAGERIIDIIWGLTTPDKKHQKDPPFLWMGYELHPDRWTVQPIELPEKESWTVNDIQKLIGKLNWASQIYAGIKVAAYVAKEIVACCDKCQLKGEAIHGQVDCSPGVWQLDCTHLEGKVILVAVHVASGYMEAEVIPTETGQETAYFILKLAGRWPVTTLPQITLWQRPIVTIKIGGQIKEALLDTGADDTVLEDMNLPGKWKPKMIGGIGGFIKVKQYDQPKFRLPIQKETWDTWWTDYWQATWIPEWEFTNTPPLVKLWYQLETEPIAGVETFYVDGASNRETKAAYAITKLQNFRVYYRDNRDPLWKGPARLLWKGEGAVVIQDNSEIKVVPRRKVKIIRDYGKRMAGDDCVAGRQDEDGTVLIGPTPVNIIGRNLLTQLGCTLNFPISPIDTVPVKLKPGMDGPRVKQWPLTEEKIKALIEICTEMEKEGKISRIGPENPYNTPIFAIKKKDGTKWRKLVDFRELNKKTQDFWEVQLGIPHPSGLKKKKSVTVLDIGDAYFSVPLDKEFRKYTAFTVPSTNNETPGVRYQYNVLPMGWKGSPAIFQCSMTKEKIYLAWVPAHKGIGGNEQIDKLVS

SEQ ID NO: 411 - protein, 768 aa, synthetic construct
  REGION 1..768: note = source = /note="Description of Artificial Sequence: Synthetic polypeptide"
QLKGEAMHGQVDCSPGIWQLDCTHLCSATEKLWVTVYYGVPVWKEATTTLASRELERFAVNPGLLNNETPGIRYQYNVLPQGWKGSPAIFPVGEIYKRWIILGLNKIVRMYSPTSIAAPPFLWMGYELHPDKWTVQPIVLPEKWETWWTEYWQATWIPEWEFVNTPPLQDSGLEVNIVTDSQYALGIIQAQPDSWTVNDIQKLVGKLNWASQIYPGIKAADPLWKGPAKLLWKGEGAVVIQDNSDRQANFLGKIWPSHKGRPLWKGPAKLLWKGEGAVVIQDNSDIAASNFTSTTVKAACWWAGIKQEFGIPYVLPEKDSWTVNDIQKLVGKLNWASQMVHQAISPRTLNAWVKVVEEKAFSPIVLPEKDSWTVNDIQKLVGKLNWASGTGPCTNVSTVQCTHGIRPVVSTQLYLKDQQLLGIWGCSGKLICTTAVPWAAAKKHQKEPPFLWMGYELHPDKWTVQPVYYRDSRDPLWKGPAKLLWKGEGAVNLLTQIGCTLNFPISPIETVPVKLKGPKEPFRDYVDRFYKTLRAEQASQEVLGPAATLEEMMTACQGVGGPGHKARDQSLKPCVKLTPLCVTLNCTDLRNTGPGIRYPLLTFGWCFKLPVEPEKVEAKIIRDYGKQMAGDDCVASRQDEDILKALGPAATLEEMMTACQGVGGPGYKAAVDLSHFLREKGGLEGAAYAAKAYDTEVHNVWATHACVPTDPNPQEAAALSEGATPQDLNTMLNTVGGHQAAMQQVDCSPGIWQLDCTHLEGKIILVAV

SEQ ID NO: 412 - protein, 39 aa, unidentified
  REGION 1..39: note = source = /note="Description of Unknown: LAMP-1 C-terminal sequence"
GSEFTLIPIAVGGALAGLVIVLIAYLVGRKRSHAGYQTI

SEQ ID NO: 413 - protein, 21 aa, unidentified
  REGION 1..21: note = source = /note="Description of Unknown: CXCL10, IP-10 sequence"
MNQTAILICCLIFLTLSGIQG

SEQ ID NO: 414 - DNA, 1104 bp, synthetic construct
  misc_feature 1..1104: note = source = /note="Description of Artificial Sequence: Synthetic polynucleotide"
  source 1..1104: mol_type = other DNA
atgggagctagagctagcgtgctgagcggaggagaactcgatcgctgggaaaagatcagactgagaccaggaggcaagaagaagtacagactgaagcacatcgtctgggcttctagagaactggaaagattcgccgtgaatccaggactgctggaaacactgaagcacattgtctgggctagcagagaactggagagatttgccgtgaatccaggactgctggaaacagcagctatctctcctagaacactgaacgcttgggtgaaagtggtggaggaaaaggcctttagcccagaagtgatccctatgtttagcgccctgtcagaaggagctacacctcaggatctgaacaccatgctgaacacagtgggaggacatcaggcagctatgcagatgctgaaggagacaattaacgaagaagccgccgagtgggatagactgcatccagtgcacgcaggacctattgctccaggacagatgagagagcctagaggaagcgatatcgcaggaacaacatctacactgcaggagcagatcggttggatgaccaataatcctcctatcccagtgggcgaaatctataagcgctggatcatcctgggactgaacaagatcgtgaggatgtacagccctaccagcatcctggatatcagacagggacctaaggagcctttcagagattacgtggacaggttctacaagacactgagagccgaacaggcttctcaggaggtgaagaattggatgaccgagacactgctggtgcagaacgctaatccagattgcaagacaattctgaaagctctgggaccagccgctacactggaagagatgatgaccgcttgtcagggagtgggaggaccaggacataaagctagagtgctggcagaagccatgtctcaggaagaagtgggattcccagtgaaacctcaggtgcctctgagacctatgacctttaagggagctctggacctgtctcacttcctgagagaaaagggaggactggaaggaacacagggatttttcccagatcagaattacacaccagagccaggaatcagattccctctgacattcggttggtgcttcaaactggtgcctctg

SEQ ID NO: 415 - DNA, 2562 bp, synthetic construct
  misc_feature 1..2562: note = source = /note="Description of Artificial Sequence: Synthetic polynucleotide"
  source 1..2562: mol_type = other DNA
ggaacagtgctggtgggacctactccagtgaatatcatcggaaggaacctgctgacacagattggttgtaccctgaacttccctatctctcctatcgagacagtgccagtgaaactgaagccaggaatggatggacctaaagtcaagcagtggcctctgacagaagagaagatcaaagccctggtggagatttgcaccgagatggagaaggagggaaagatcagcaagatcggcccagagaatccttacaacaccccagtgttcgccatcaagaagaaggatagcaccaagtggagaaagctggtggatttcagggagctgaacaagagaacccaggatttttgggaggtgcagctgggtattccacatcctgccggactgaaaaagaagaaaagcgtgacagtgctggacgtgggagacgcttatttcagcgtgcctctggataaggacttcagaaagtacaccgccttcaccatcccttctatcaacaacgagaccccaggaatcagataccagtacaacgtgctgcctcaaggttggaaaggatctccagccatctttcagagcagcatgacaacagtgaaggcagcttgttggtgggcaggaattaagcaggagttcggcatcccttacaatcctcagtctcagggagtggtggaatctatgaacaaggagctgaagaagatcatcggacaggtgagagatcaggccgaacatctgaagacagcagtgcaaatggccgtgttcatccacaacttcaagagaaagggcggcattggaggctattctgccggagagagaattgtggacatcatcaacgtgtcaacagtccagtgtacacacggaatcagaccagtcgtgtctacacaactgctgctgaacggatctctggccgaagagaagagaagagtggtgcagagagagaaaagagcagtgggaatcggagctatgtttctgggatttctgggcgcagcaggatctacaatgggagcagcttctatcacactgacagtgcaggctagacaactgctgagcggaattgtgcagcagcagaataacctgctgagagctatcgaagctcagcaacatctgctgcaactcaccgtctggggaattaagcaactgcaagctagagtgctggcagtggaaagatacctgaaggatcagcaactgctgggaatttggggttgctcaggcaagctgatttgcacaaccgtggccaaagagattgtggcttcttgcgacaagtgtcagctgaaaggagaagctatgcacggacaagtggattgttctccaggaatttggcagctggattgtacacacctggagggaaagattattctggtggcagtgcacgtggccagcggatatattgaagccgaggtgattccagcagaaacaggacaggaaacagcctattttctcctgaaactggcaggtaggtggccagtgaaaaccctctgggtgacagtgtactacggagtcccagtctggaaagaagcagctttccctcagattactctctggcagagacctctggtgacaatcaagatcggcggacagctgaaagaagctctgctggatacaggagcagacgatacagtgctggaagaaatgaacctgccaggtagatggaagcctaagatgatcggaggcatcggaggattcatcaaggtgagacagtacgaccaagcagcagcagctcataacgtctgggctacacacgcttgcgtgcctacagatcctaatcctcaggaagccatcaccaagatccagaatttcagggtgtactacagggacagcagagatcctctctggaaaggaccagctaaactgctgtggaaaggagaaggagcagtggtgatccaggataacagcgacatcaaggtggtgcctagaagaaaggccaagatcatcagggactacggaaagcaaatggcaggagacgattgcgtggcttctagacaggacgaggatcccaagttcaagctgcctattcagaaggagacttgggagacttggtggacagagtattggcaagcaacttggatccccgagtgggaatttgtgaatacccctcctctggtcaagctctggtatcagctggaaaaggagcctatcgtgggagccgaaacattttacgtggacggagcagctaatagagagacaaaagccgccaaggagaaagtgtatctggcttgggtgccagctcataaaggaatcggaggaaacgagcaggtggataaactggtgtcttggggctttaccacaccagataagaagcaccagaaggagccaccatttctctggatgggatacgaactgcacccagataagtggacagtccagcctattgtgctgccagaaaaggactcttggacagtgaacgacatccagaaactggtgggaaagctgaattgggcctctcagatctacccaggcatcaaggtgatcgtgatctaccagtacatggacgatctgtacgtgggatcagatctggagatcggacagcacagaatgagggacaattggagaagcgagctgtacaagtacaaggtggtg

SEQ ID NO: 416 - DNA, 1144 bp, synthetic construct
  misc_feature 1..1144: note = source = /note="Description of Artificial Sequence: Synthetic polynucleotide"
  source 1..1144: mol_type = other DNA
taccagtataacgtgctgcctcagggagcttctagagaactggagagattcgcagtgaacccaggactcctctggattatcctgggactgaacaagatcgtgaggatgtactctcctacctctattgccgctagaacactgaacgcttgggtgaaggtcttcctctggatgggatacgaactgcatctgacctttggttggtgctttaagctccctctctggaaaggaccagctaagctggtgacagtgtattacggagtgccagtggcagctctcctctggaaaggagaaggagcagtggcagcagctaaactggtgggaaagctgaattgggccaaactcctctggaagggagaaggagccaccctgaattttcctatcagccctatttggcaggctacttggattccagagtggaaagcagcttgttggtgggcaggaatcagacaggccaacttcctgggcaagatttggccttctcacaaaggaagaaacgtctgggctacacacgcttgcgtggcagcagaaatgatgacagcttgtcagggagtgtctacagtccagtgtacacacggaatcgcagctaaacagatggcaggagacgattgcgtggcagcttggcagctggattgtacacacctggagtacaaggcagcagtggatctgtctcactttctgagagaaaaaggaggactggaaggagcagcttactacatggacgatctgtacgtgggatcaggacaggtggattgttcaccaggaatcgctacactggaggaaatgatgaccgcagaactgcatccagataagtggaccgtctggacagtgaacgatatccagaagctgggcatttggggttgtagcggaaaactgaccgtgaacgatatccagaagctggtgatcgtgaccgattctcagtacgctctgtacgtggacagattctacaagaccctgtacgtggacaggttctacaagacactgagagccgaacaggcttctcaggaagtggatctgaacaccatgctgaacaccgtgaaactgacacctctctgcgtgacactgtatcagtacatggacgacctgtacgtggtgatctaccagtacatggacgatctctggatcatcctgggactgaacaagatcg

SEQ ID NO: 417 - DNA, 1518 bp, synthetic construct
  misc_feature 1..1518: note = source = /note="Description of Artificial Sequence: Synthetic polynucleotide"
  source 1..1518: mol_type = other DNA
agaacactgaacgcttgggtgaaggtgagagagaagagagacctgaacaccatgctgaacaccgtgagagaaaagaggtggatcatcctgggactgaacaagatcagggagaagaggtacgtggacaggttctacaagacactgagagagaagagagccacactggaagagatgatgaccgctagagagaagagagagatgatgaccgcttgtcagggagtgagagagaagagaaccctgaacttccccatctctcctatcagggagaagaggtaccagtacaacgtgctgcctcagggaagagaaaagagagtgatctaccagtacatggacgacctgagagagaagaggtaccagtacatggacgatctgtacgtgagggagaagagatacatggacgacctgtacgtgggatcaagagagaagagattcctctggatgggctacgagctgcatagagagaagagagagctgcacccagataagtggacagtgagagaaaagcgctggacagtgaacgacatccagaagctgagagagaagaggacagtgaacgacatccagaagctggtgagagagaagaggaagctggtgggaaaactgaattgggctagggaaaaaaggtggcaggctacttggattccagagtggagagagaagaggatcgtgacagatagccagtacgctctgagagagaaaagaggacaggtggattgctctccaggaatcagagagaagagatggcagctggattgtacacacctggagagagagaagaggaaagcagcttgttggtgggcaggaattcgggaaaaaagacctctctggaaaggaccagccaagctgagagagaagagaaaactcctctggaagggcgaaggagctagagaaaagagactcctctggaaaggagaaggcgcagtgagagagaagagaaaacagatggccggagacgattgcgtgagagaaaagagagtgaccgtgtattacggagtgccagtgagagaaaagagaaacgtctgggctacacacgcttgcgtgagagagaagagaaagctgacacctctgtgcgtgacactgagagaaaagagaagcaccgtgcagtgtacacacggaattagggagaagagaggcatttggggttgttcaggaaagctgagagagaagaggctgacattcggttggtgtttcaagctgagggagaagagagcctctagagaactggagagattcgcagtgaatccaggactgctgagagaaaagcgctggattatcctgggactgaacaagatcgtgaggatgtacagccctacaagcatcagagagaagaggtacgtggacagattctacaagaccctgagagccgaacaggcatctcaggaagtgagagagaagagaaggcaggctaacttcctgggaaagatttggcctagccacaagggaagaagagagaagagatacaaggccgcagtggatctgtctcactttctgagagagaaaggaggactggaaggagga

SEQ ID NO: 418 - DNA, 2784 bp, synthetic construct
  misc_feature 1..2784: note = source = /note="Description of Artificial Sequence: Synthetic polynucleotide"
  source 1..2784: mol_type = other DNA
atggctcctagaagcgctagaagacctctgctgctgctgctgctgctgctgctgctgggactgatgcattgcgcttcagcagctatgttcatggtgaagaacggcaacggaacagcttgtatcatggccaatttcagcgccgcttttagcgtgaattacgacaccaagagcggacctaagaacatgacactggatctgccttcagacgctacagtggtgctgaatagaagctcttgcggaaaggagaatacctccgatccttctctggtgatcgcttttggcagaggacacacactgacactgaacttcaccagaaacgccaccagatactcagtgcagctgatgagcttcgtgtacaacctgagcgatacccatctgtttcctaacgctagcagcaaggagatcaagacagtggagtctatcaccgacatcagagccgatatcgacaagaaataccgctgcgtgtcaggaacacaggtgcacatgaacaacgtgacagtgacactgcacgacgccacaattcaggcctatctgagcaatagcagctttagcagaggcgaaactaggtgtgagcaggatagaccttctcctacaacagctcctccagctcctccttctccttctccttctccagtgcctaaatctcctagcgtggataagtacaacgtgagcggaacaaacggcacttgtctgctggcttctatgggactgcagctgaatctgacatacgagaggaaggacaacaccacagtgacaagactgctgaacatcaaccccaacaaaacaagcgctagcggatcttgcggagctcatctggtgacactggaactgcattcagagggaacaacagtgctgctgtttcagttcggaatgaacgcctctagcagcagattcttcctgcagggtattcagctgaatacactgctgccagatgctagagatccagcctttaaagccgctaatggatctctgagagctctgcaggctacagtgggaaatagctacaagtgcaacgccgaagaacacgtgagagtgacaaaagccttcagcgtgaacatctttaaggtctgggtgcaggcatttaaagtggagggaggccagtttggaagcgtcgaagagtgtctgctggacgaaaatagcctggaagacatcagaacactgaacgcttgggtgaaggtgagagagaagagagacctgaacaccatgctgaacaccgtgagagaaaagaggtggatcatcctgggactgaacaagatcagggagaagaggtacgtggacaggttctacaagacactgagagagaagagagccacactggaagagatgatgaccgctagagagaagagagagatgatgaccgcttgtcagggagtgagagagaagagaaccctgaacttccccatctctcctatcagggagaagaggtaccagtacaacgtgctgcctcagggaagagaaaagagagtgatctaccagtacatggacgacctgagagagaagaggtaccagtacatggacgatctgtacgtgagggagaagagatacatggacgacctgtacgtgggatcaagagagaagagattcctctggatgggctacgagctgcatagagagaagagagagctgcacccagataagtggacagtgagagaaaagcgctggacagtgaacgacatccagaagctgagagagaagaggacagtgaacgacatccagaagctggtgagagagaagaggaagctggtgggaaaactgaattgggctagggaaaaaaggtggcaggctacttggattccagagtggagagagaagaggatcgtgacagatagccagtacgctctgagagagaaaagaggacaggtggattgctctccaggaatcagagagaagagatggcagctggattgtacacacctggagagagagaagaggaaagcagcttgttggtgggcaggaattcgggaaaaaagacctctctggaaaggaccagccaagctgagagagaagagaaaactcctctggaagggcgaaggagctagagaaaagagactcctctggaaaggagaaggcgcagtgagagagaagagaaaacagatggccggagacgattgcgtgagagaaaagagagtgaccgtgtattacggagtgccagtgagagaaaagagaaacgtctgggctacacacgcttgcgtgagagagaagagaaagctgacacctctgtgcgtgacactgagagaaaagagaagcaccgtgcagtgtacacacggaattagggagaagagaggcatttggggttgttcaggaaagctgagagagaagaggctgacattcggttggtgtttcaagctgagggagaagagagcctctagagaactggagagattcgcagtgaatccaggactgctgagagaaaagcgctggattatcctgggactgaacaagatcgtgaggatgtacagccctacaagcatcagagagaagaggtacgtggacagattctacaagaccctgagagccgaacaggcatctcaggaagtgagagagaagagaaggcaggctaacttcctgggaaagatttggcctagccacaagggaagaagagagaagagatacaaggccgcagtggatctgtctcactttctgagagagaaaggaggactggaaggaggaagcgagtttaccctgattccaattgccgtgggaggagctctggcaggactggtgattgtgctgatcgcatacctggtgggaagaaagagatctcacgccggatatcagaccatc

SEQ ID NO: 419-420 - skipped (2 entries)

SEQ ID NO: 421 - protein, 4 aa, synthetic construct
  REGION 1..4: note = source = /note="Description of Artificial Sequence: Synthetic peptide"
GGGS

SEQ ID NO: 422 - protein, 706 aa, synthetic construct
  REGION 1..706: note = source = /note="Description of Artificial Sequence: Synthetic polypeptide"
ICGHKAIGTVLVGPTPVNIIGRNLLTQLGCTLNFPISPIETVPVKLKPGMDGPKVKQWPLTEEKIKALDPLWKGPAKLLWKGEGAVVIQDNSDIKVVPRRKAKIIRDYGKQMAAAYSDIAGTTSTLQEQITWMTNNPPIPVGEIYKRWIILGLNKIVRMYSPVSILDIRQGPKEPFRDYVDRFYKTLRAEQASQEVKNWMTETLLVQNANPDCKTILKALGPAATLEEMMTACQGVGGPGHKARVLAEAMSQVTNSATLNKRTQDFWEVQLGIPHPAGLKKKKSNFTSTTVKAACWWAGIKQEFGIPYNPQSAYFSVPLDKEFRKYTAFTIPSINNEDTVLEEMNLPGKWKPKMIGGIGGFIKVRQYDQISKIGPENPYNTPIFAIKKKDSTKWAAGKKKYRLKHLVWVSRELERFAVNPGGKKKYRLKHLVWASRELERFAVNPGAEHLKTAVQMAVFIHNFKRKGGIGGAAGQMVHQAISPRTLNAWVKVVEEKAFSPEVIPMFSALAEGATPQDLNTMLNTVGGHQARWIILGLNKTVRMYSPVSILDIRQGPKEPFRDYVDRFYKTLRAEQASQEVKNWMTETLLVQNANPDCKTILKALGPAATLEEMMTACQGVGGPGHKARVLAEAMSQVTNSATQLKGEAMHGQVDCSPGIWQLDCTHLEGKVILVAVHVASGYIEAEVIPAETGQETAYFLLKLAGR

SEQ ID NO: 423 - protein, 484 aa, synthetic construct
  REGION 1..484: note = source = /note="Description of Artificial Sequence: Synthetic polypeptide"
SNFTSTTVKVACWWAGIKQEFGIPYAASNFTSTTVKAACWWAGVKQEFGIPYAASNFTSTTVKAACWWAGIKQEFGIPYPLRPMTYKAAVDLSHFLKEKGGLEGPLRPMTYKAAVDLSFFLKEKGGLEGPLRPMTYKGAFDLSFFLKEKGGLEGPLRPMTYKAAFDLSFFLKEKGGLEGPLRPMTYKAAFDLSHFLKEKGGLEGPLRPMTYKGALDLSHFLKEKGGLEGQLKGEAMHGQVDCSPGIWQLDCTHLEEKIILVAVHVASGYIEAEVIPAETGQETAYMVHQAISPRTLNAWVKVVEEKAFSPLDCTHLEGKVILVAVHVASGYIEAEICGHKAIGTVLVGPTPVNIIGRNLLTQIGCTLNFPISPIETVPVKLKDPLWKGPAKLLWKGEGAVVIQDNSDIDPLWKGPAKLLWKGEGVVVIQDNSDIMVHQAISPRTLNALVKVVEEKAFSPICGHKAIGTVLVGSTPVNIIGRNLL

SEQ ID NO: 424 - protein, 506 aa, synthetic construct
  REGION 1..506: note = source = /note="Description of Artificial Sequence: Synthetic polypeptide"
IRTLNAWVKVREKRDLNTMLNTVREKRWIILGLNKIREKRYVDRFYKTLREKRATLEEMMTAREKREMMTACQGVREKRTLNFPISPIREKRYQYNVLPQGREKRVIYQYMDDLREKRYQYMDDLYVREKRYMDDLYVGSREKRFLWMGYELHREKRELHPDKWTVREKRWTVNDIQKLREKRTVNDIQKLVREKRKLVGKLNWAREKRWQATWIPEWREKRIVTDSQYALREKRGQVDCSPGIREKRWQLDCTHLEREKRKAACWWAGIREKRPLWKGPAKLREKRKLLWKGEGAREKRLLWKGEGAVREKRKQMAGDDCVREKRVTVYYGVPVREKRNVWATHACVREKRKLTPLCVTLREKRSTVQCTHGIREKRGIWGCSGKLREKRLTFGWCFKLREKRASRELERFAVNPGLLREKRWIILGLNKIVRMYSPTSIREKRYVDRFYKTLRAEQASQEVREKRRQANFLGKIWPSHKGRREKRYKAAVDLSHFLREKGGLEG

SEQ ID NO: 425 - protein, 80 aa, Human immunodeficiency virus
AVKAACWWAGVKQEFGIPYNTQSQGVVESMNNELKKIIGQIRDQAEHLKTAVQMAVLIHNFKRKGGIGEYSAGERIIDII

SEQ ID NO: 426 - protein, 81 aa, Human immunodeficiency virus
AVKAACWWAGVKQEFGIPYNTQSQGVVESMNNELKKIIGQIRDQAEHLKTAVQMAVLIHNFKRKGGIGEYSAGERIIDIIA

SEQ ID NO: 427 - protein, 81 aa, Human immunodeficiency virus
VAKEIVACCDKCQLKGEAIHGQVDCSPGVWQLDCTHLEGKVILVAVHVASGYIEAEIIPTETGQETAYFILKLAGRWPVTT

SEQ ID NO: 428 - protein, 256 aa, synthetic construct
  REGION 1..256: note = source = /note="Description of Artificial Sequence: Synthetic polypeptide"
LKHIVWASRELERFAVNPGLLETVSQNYPIVQNISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQ

SEQ ID NO: 429 - protein, 218 aa, synthetic construct
  REGION 1..218: note = source = /note="Description of Artificial Sequence: Synthetic polypeptide"
LPGRWKPKMIGGIGGFIKVRQYDQGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSWGFTTPDKKHQKEPPFLWMGYELHPDKWTVQPI

SEQ ID NO: 430 - protein, 368 aa, synthetic construct
  REGION 1..368: note = source = /note="Description of Artificial Sequence: Synthetic polypeptide"
MGARASVLSGGELDRWEKIRLRPGGKKKYRLKHIVWASRELERFAVNPGLLETLKHIVWASRELERFAVNPGLLETAAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQEEVGFPVKPQVPLRPMTFKGALDLSHFLREKGGLEGTQGFFPDQNYTPEPGIRFPLTFGWCFKLVPL

SEQ ID NO: 431 - protein, 381 aa, synthetic construct
  REGION 1..381: note = source = /note="Description of Artificial Sequence: Synthetic polypeptide"
YQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLYVDRFYKTLRAEQASQEVDLNTMLNTVKLTPLCVTLYQYMDDLYVVIYQYMDDLWIILGLNKI

SEQ ID NO: 432 - protein, 399 aa, synthetic construct
  REGION 1..399: note = source = /note="Description of Artificial Sequence: Synthetic polypeptide"
MWLQSLLLLGTVACSISVYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLYVDRFYKTLRAEQASQEVDLNTMLNTVKLTPLCVTLYQYMDDLYVVIYQYMDDLWIILGLNKI

SEQ ID NO: 433 - protein, 404 aa, synthetic construct
  REGION 1..404: note = source = /note="Description of Artificial Sequence: Synthetic polypeptide"
MDAMKRGLCCVLLLCGAVFVSARYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLYVDRFYKTLRAEQASQEVDLNTMLNTVKLTPLCVTLYQYMDDLYVVIYQYMDDLWIILGLNKI

SEQ ID NO: 434 - protein, 488 aa, synthetic construct
  REGION 1..488: note = source = /note="Description of Artificial Sequence: Synthetic polypeptide"
MNPSAAVIFCLILLGLSGTQGILDMAQPVGINTSTTCCYRFINKKIPKQRLESYRRTTSSHCPREAVIFKTKLDKEICADPTQKWVQDFMKHLDKKTQTPKLASAGAYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLYVDRFYKTLRAEQASQEVDLNTMLNTVKLTPLCVTLYQYMDDLYVVIYQYMDDLWIILGLNKI

SEQ ID NO: 435 - protein, 412 aa, synthetic construct
  REGION 1..412: note = source = /note="Description of Artificial Sequence: Synthetic polypeptide"
MRKAAVSHWQQQSYLDSGIHSGATTTAPSLSYQYNVLPQGASRELERFAVNPGLLWIILGLNKIVRMYSPTSIAARTLNAWVKVFLWMGYELHLTFGWCFKLPLWKGPAKLVTVYYGVPVAALLWKGEGAVAAAKLVGKLNWAKLLWKGEGATLNFPISPIWQATWIPEWKAACWWAGIRQANFLGKIWPSHKGRNVWATHACVAAEMMTACQGVSTVQCTHGIAAKQMAGDDCVAAWQLDCTHLEYKAAVDLSHFLREKGGLEGAAYYMDDLYVGSGQVDCSPGIATLEEMMTAELHPDKWTVWTVNDIQKLGIWGCSGKLTVNDIQKLVIVTDSQYALYVDRFYKTLYVDRFYKTLRAEQASQEVDLNTMLNTVKLTPLCVTLYQYMDDLYVVIYQYMDDLWIILGLNKI

SEQ ID NO: 436 - protein, 9 aa, synthetic construct
  REGION 1..9: note = source = /note="Description of Artificial Sequence: Synthetic peptide"
DYKDDDDKL

SEQ ID NO: 437 - protein, 27 aa, Human immunodeficiency virus
MRVKEKYQHLWRWGWRWGTMLLGMLMI

SEQ ID NO: 438 - protein, 6 aa, Human immunodeficiency virus
FCASDA

SEQ ID NO: 439 - protein, 29 aa, Human immunodeficiency virus
VVLVNVTENFNMWKNDMVEQMHEDIISLW

SEQ ID NO: 440 - protein, 96 aa, Human immunodeficiency virus
NTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFN

SEQ ID NO: 441 - protein, 206 aa, Human immunodeficiency virus
EVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGD

SEQ ID NO: 442 - protein, 12 aa, Human immunodeficiency virus
KIEPLGVAPTKA

SEQ ID NO: 443 - protein, 246 aa, Human immunodeficiency virus
NASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 444 - protein, 30 aa, Human immunodeficiency virus
MGARASVLSGGELDRWEKIRLRPGGKKKYK

SEQ ID NO: 445 - protein, 74 aa, Human immunodeficiency virus
SEGCRQILGQLQPSLQTGSEELRSLYNTVATLYCVHQRIEIKDTKEALDKIEEEQNKSKKKAQQAAADTGHSNQ

SEQ ID NO: 446 - protein, 9 aa, Human immunodeficiency virus
IQGQMVHQA

SEQ ID NO: 447 - protein, 59 aa, Human immunodeficiency virus
VTNSATIMMQRGNFRNQRKIVKCFNCGKEGHTARNCRAPRKKGCWKCGKEGHQMKDCTE

SEQ ID NO: 448 - protein, 56 aa, Human immunodeficiency virus
PGNFLQSRPEPTAPPEESFRSGVETTTPPQKQEPIDKELYPLTSLRSLFGNDPSSQ

SEQ ID NO: 449 - protein, 63 aa, Human immunodeficiency virus
MGGKWSKSSVIGWPTVRERMRRAEPAADRVGAASRDLEKHGAITSSNTAATNAACAWLEAQEE

SEQ ID NO: 450 - protein, 17 aa, Human immunodeficiency virus
LIHSQRRQDILDLWIYH

SEQ ID NO: 451 - protein, 76 aa, Human immunodeficiency virus
PGVRYPLTFGWCYKLVPVEPDKIEEANKGENTSLLHPVSLHGMDDPEREVLEWRFDSRLAFHHVARELHPEYFKNC

SEQ ID NO: 452 - protein, 55 aa, Human immunodeficiency virus
FFREDLAFLQGKAREFSSEQTRANSPTRRELQVWGRDNNSPSEAGADRQGTVSFN

SEQ ID NO: 453 - protein, 11 aa, Human immunodeficiency virus
ILIEICGHKAI

SEQ ID NO: 454 - protein, 5 aa, Human immunodeficiency virus
KILEP

SEQ ID NO: 455 - protein, 12 aa, Human immunodeficiency virus
TKIEELRQHLLR

SEQ ID NO: 456 - protein, 109 aa, Human immunodeficiency virus
QLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKT

SEQ ID NO: 457 - protein, 35 aa, Human immunodeficiency virus
LGKAGYVTNRGRQKVVTLTDTTNQKTELQAIYLAL

SEQ ID NO: 458 - protein, 16 aa, Human immunodeficiency virus
QSESELVNQIIEQLIK

SEQ ID NO: 459 - protein, 37 aa, Human immunodeficiency virus
GIRKVLFLDGIDKAQDEHEKYHSNWRAMASDFNLPPV

SEQ ID NO: 460 - protein, 6 aa, Human immunodeficiency virus
IHTDNG

SEQ ID NO: 461 - protein, 10 aa, Human immunodeficiency virus
TDIQTKELQK

SEQ ID NO: 462 - protein, 4 aa, synthetic construct
  REGION 1..4: note = source = /note="Description of Artificial Sequence: Synthetic peptide"
YMDD

SEQ ID NO: 463 - protein, 4 aa, synthetic construct
  REGION 1..4: note = source = /note="Description of Artificial Sequence: Synthetic peptide"
YVDD

SEQ ID NO: 464 - protein, 9 aa, synthetic construct
  REGION 1..9: note = source = /note="Description of Artificial Sequence: Synthetic peptide"
AIIIIIIIS

SEQ ID NO: 465 - protein, 9 aa, synthetic construct
  REGION 1..9: note = source = /note="Description of Artificial Sequence: Synthetic peptide"
MIIIIIIII

SEQ ID NO: 466 - protein, 9 aa, synthetic construct
  REGION 1..9: note = source = /note="Description of Artificial Sequence: Synthetic peptide"
IIIIIIISK

SEQ ID NO: 467 - protein, 9 aa, synthetic construct
  REGION 1..9: note = source = /note="Description of Artificial Sequence: Synthetic peptide"
IIIIIIIIR

SEQ ID NO: 468 - protein, 10 aa, synthetic construct
  REGION 1..10: note = source = /note="Description of Artificial Sequence: Synthetic peptide"
AIIIIIIISK

SEQ ID NO: 469 - protein, 10 aa, synthetic construct
  REGION 1..10: note = source = /note="Description of Artificial Sequence: Synthetic peptide"
MIIIIIIIIR

SEQ ID NO: 470 - protein, 4 aa, synthetic construct
  SITE 1..4: note = /note="This sequence may encompass 2-4 residues"
  REGION 1..4: note = source = /note="Description of Artificial Sequence: Synthetic peptide"
AAAA

SEQ ID NO: 471 - protein, 10 aa, synthetic construct
  REGION 1..10: note = source = /note="Description of Artificial Sequence: Synthetic peptide"
GIIIIIIIIK

SEQ ID NO: 472 - protein, 10 aa, synthetic construct
  REGION 1..10: note = source = /note="Description of Artificial Sequence: Synthetic peptide"
AIIIIIIIIH

SEQ ID NO: 473 - protein, 10 aa, synthetic construct
  REGION 1..10: note = source = /note="Description of Artificial Sequence: Synthetic peptide"
GIIIIIIIIH

SEQ ID NO: 474 - protein, 10 aa, synthetic construct
  REGION 1..10: note = source = /note="Description of Artificial Sequence: Synthetic peptide"
AIIIIIIIIK

SEQ ID NO: 475 - protein, 27 aa, synthetic construct
  REGION 1..27: note = source = /note="Description of Artificial Sequence: Synthetic peptide"
QNLQGQMVHQAISPRTLNAWVKVVKAF

SEQ ID NO: 476 - protein, 27 aa, synthetic construct
  REGION 1..27: note = source = /note="Description of Artificial Sequence: Synthetic peptide"
QNLQGQMVHQAISPRTLNAWVKVEKAF

SEQ ID NO: 477 - protein, 27 aa, synthetic construct
  REGION 1..27: note = source = /note="Description of Artificial Sequence: Synthetic peptide"
QNLQGQMVHQAISPRTLNAWVKIVKAF

SEQ ID NO: 478 - protein, 27 aa, synthetic construct
  REGION 1..27: note = source = /note="Description of Artificial Sequence: Synthetic peptide"
QNIQGQMVHQAISPRTLNAWVKIEKAF

SEQ ID NO: 479 - protein, 27 aa, synthetic construct
  REGION 1..27: note = source = /note="Description of Artificial Sequence: Synthetic peptide"
QNIQGQMVHQAISPRTLNAWVKVVKAF

SEQ ID NO: 480 - protein, 27 aa, synthetic construct
  REGION 1..27: note = source = /note="Description of Artificial Sequence: Synthetic peptide"
PNIQGQMVHQAISPRTLNAWVKIVKAF

SEQ ID NO: 481 - protein, 9 aa, synthetic construct
  REGION 1..9: note = source = /note="Description of Artificial Sequence: Synthetic peptide"
QNLQGQMVH

SEQ ID NO: 482 - protein, 9 aa, synthetic construct
  REGION 1..9: note = source = /note="Description of Artificial Sequence: Synthetic peptide"
QNIQGQMVH

SEQ ID NO: 483 - protein, 9 aa, synthetic construct
  REGION 1..9: note = source = /note="Description of Artificial Sequence: Synthetic peptide"
PNIQGQMVH

SEQ ID NO: 484 - protein, 10 aa, synthetic construct
  REGION 1..10: note = source = /note="Description of Artificial Sequence: Synthetic peptide"
GIIIIIIIIR

SEQ ID NO: 485 - protein, 9 aa, synthetic construct
  REGION 1..9: note = source = /note="Description of Artificial Sequence: Synthetic peptide"
GIIIIIIII

SEQ ID NO: 486 - protein, 9 aa, synthetic construct
  REGION 1..9: note = source = /note="Description of Artificial Sequence: Synthetic peptide"
AIIIIIIII

SEQ ID NO: 487 - protein, 9 aa, synthetic construct
  REGION 1..9: note = source = /note="Description of Artificial Sequence: Synthetic peptide"
IIIIIIIIK

SEQ ID NO: 488 - protein, 9 aa, synthetic construct
  REGION 1..9: note = source = /note="Description of Artificial Sequence: Synthetic peptide"
IIIIIIIIH

SEQ ID NO: 489 - protein, 9 aa, synthetic construct
  REGION 1..9: note = source = /note="Description of Artificial Sequence: Synthetic peptide"
QAISPRTLN

SEQ ID NO: 490 - protein, 27 aa, synthetic construct
  REGION 1..27: note = source = /note="Description of Artificial Sequence: Synthetic peptide"
QNLQGQMVHQATSPRTLNAWVKVVKAF

SEQ ID NO: 491 - protein, 9 aa, synthetic construct
  REGION 1..9: note = source = /note="Description of Artificial Sequence: Synthetic peptide"
QATSPRTLN

SEQ ID NO: 492 - protein, 9 aa, Human immunodeficiency virus
HPPQAGPVA

SEQ ID NO: 493 - protein, 9 aa, Homo sapiens
VPLQAGPVQ

SEQ ID NO: 494 - protein, 9 aa, Homo sapiens
MPGQAGPVG

SEQ ID NO: 495 - protein, 9 aa, Homo sapiens
QYRQAGPVI

SEQ ID NO: 496 - protein, 9 aa, Homo sapiens
TATQAGLVF

SEQ ID NO: 497 - protein, 9 aa, Homo sapiens
HTKQAGLVV

SEQ ID NO: 498 - protein, 9 aa, Homo sapiens
GTRQAGLVA

SEQ ID NO: 499 - protein, 9 aa, Human immunodeficiency virus
ISPRTLNAW

SEQ ID NO: 500 - protein, 9 aa, Human immunodeficiency virus
MFSALSEGA

SEQ ID NO: 501 - protein, 9 aa, Human immunodeficiency virus
ILDIRQGPK

SEQ ID NO: 502 - protein, 9 aa, Human immunodeficiency virus
LSPRTLNAW

SEQ ID NO: 503 - protein, 9 aa, Human immunodeficiency virus
MFSALSYGA

SEQ ID NO: 504 - protein, 9 aa, Human immunodeficiency virus
ILDIRRGPK

SEQ ID NO: 505 - protein, 9 aa, Human immunodeficiency virus
ATLEEMVTA

SEQ ID NO: 506 - protein, 9 aa, synthetic construct
  REGION 1..9: note = source = /note="Description of Artificial Sequence: Synthetic peptide"
ILDTRQGPK

SEQ ID NO: 507 - protein, 9 aa, synthetic construct
  REGION 1..9: note = source = /note="Description of Artificial Sequence: Synthetic peptide"
ATLVEMMTA

SEQ ID NO: 508 - protein, 9 aa, synthetic construct
  REGION 1..9: note = source = /note="Description of Artificial Sequence: Synthetic peptide"
ILSIRQGPK

SEQ ID NO: 509 - protein, 9 aa, synthetic construct
  REGION 1..9: note = source = /note="Description of Artificial Sequence: Synthetic peptide"
ATSEEMMTA

SEQ ID NO: 510 - protein, 9 aa, synthetic construct
  MOD_RES 8: note = Any amino acid
  REGION 1..9: note = source = /note="Description of Artificial Sequence: Synthetic peptide"
ILDIRQGXK

SEQ ID NO: 511 - protein, 109 aa, Human immunodeficiency virus
KRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTAVPW

SEQ ID NO: 512 - protein, 21 aa, Human immunodeficiency virus
YLKDQQLLGIWGCSGKLICTT

SEQ ID NO: 513 - protein, 35 aa, Human immunodeficiency virus
EEVGFPVKPQVPLRPMTFKGALDLSHFLREKGGLE

SEQ ID NO: 514 - protein, 14 aa, Human immunodeficiency virus
TQGFFPDWQNYTPE

SEQ ID NO: 515 - protein, 18 aa, Human immunodeficiency virus
PGIRFPLTFGWCFKLVPL

SEQ ID NO: 516 - protein, 21 aa, Human immunodeficiency virus
VTVLDVGDAYFSVPLDKDFRK

SEQ ID NO: 517 - protein, 8 aa, Human immunodeficiency virus
YTAFTIPS

SEQ ID NO: 518 - protein, 8 aa, Human immunodeficiency virus
VIYQYMDD

SEQ ID NO: 519 - protein, 78 aa, Human immunodeficiency virus
TVKAACWWAGIKQEFGIPYNPQSQGVVESMNKELKKIIGQVRDQAEHLKTAVQMAVFIHNFKRKGGIGGYSAGERIVD